SEQUENCE LISTING (51 sequences)

SEQ ID NO: 1 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSERPGACNRCTEGVGYTNASNNLFACLPCTACKSDEEERSPCTTTRNTACQCKPGTFRNDNSAEMCRKCSTGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAKEKIQDLLVDSGKFIYLEDGTGSAVSLE

SEQ ID NO: 2 - protein, 299 aa, Homo sapiens [sequence omitted]

SEQ ID NO: 3 - protein, 411 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 4 - protein, 386 aa, Homo sapiens
MGLWGQSVPTASSARAGRYPGARTASGTRPWLLDPKILKFVVFIVAVLLPVRVDSATIPRQDEVPQQTVAPQQQRRSLKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESAASSTGKTPAAEETVTTILGMLASPYHYLIIIVVLVIILAVVVVGFSCRKKFISYLKGICSGGGGGPERVHRVLFRRRSCPSRVPGAEDNARNETLSNRYLQPTQVSEQEIQGQELAELTGVTVESPEEPQRLLEQAEAEGCQRRRLLVPVNDADSADISTLLDASATLEEGHAKETIQDQLVGSEKLFYEEDEAGSATSCL

SEQ ID NO: 5 - protein, 281 aa, Homo sapiens [sequence omitted]

SEQ ID NO: 6 - DNA, 23 bp, Homo sapiens
caggtgcagctggtgcagtctgg

SEQ ID NO: 7 - DNA, 23 bp, Homo sapiens
caggtcaacttaagggagtctgg

SEQ ID NO: 8 - DNA, 23 bp, Homo sapiens
gaggtgcagctggtggagtctgg

SEQ ID NO: 9 - DNA, 23 bp, Homo sapiens
caggtgcagctgcaggagtcggg

SEQ ID NO: 10 - DNA, 23 bp, Homo sapiens
gaggtgcagctgttgcagtctgc

SEQ ID NO: 11 - DNA, 23 bp, Homo sapiens
caggtacagctgcagcagtcagg

SEQ ID NO: 12 - DNA, 24 bp, Homo sapiens
tgaggagacggtgaccagggtgcc

SEQ ID NO: 13 - DNA, 24 bp, Homo sapiens
tgaagagacggtgaccattgtccc

SEQ ID NO: 14 - DNA, 24 bp, Homo sapiens
tgaggagacggtgaccagggttcc

SEQ ID NO: 15 - DNA, 24 bp, Homo sapiens
tgaggagacggtgaccgtggtccc

SEQ ID NO: 16 - DNA, 23 bp, Homo sapiens
gacatccagatgacccagtctcc

SEQ ID NO: 17 - DNA, 23 bp, Homo sapiens
gatgttgtgatgactcagtctcc

SEQ ID NO: 18 - DNA, 23 bp, Homo sapiens
gatattgtgatgactcagtctcc

SEQ ID NO: 19 - DNA, 23 bp, Homo sapiens
gaaattgtgttgacgcagtctcc

SEQ ID NO: 20 - DNA, 23 bp, Homo sapiens
gacatcgtgatgacccagtctcc

SEQ ID NO: 21 - DNA, 23 bp, Homo sapiens
gaaacgacactcacgcagtctcc

SEQ ID NO: 22 - DNA, 23 bp, Homo sapiens
gaaattgtgctgactcagtctcc

SEQ ID NO: 23 - DNA, 23 bp, Homo sapiens
cagtctgtgttgacgcagccgcc

SEQ ID NO: 24 - DNA, 23 bp, Homo sapiens
cagtctgccctgactcagcctgc

SEQ ID NO: 25 - DNA, 23 bp, Homo sapiens
tcctatgtgctgactcagccacc

SEQ ID NO: 26 - DNA, 23 bp, Homo sapiens
tcttctgagctgactcaggaccc

SEQ ID NO: 27 - DNA, 23 bp, Homo sapiens
cacgttatactgactcaaccgcc

SEQ ID NO: 28 - DNA, 23 bp, Homo sapiens
caggctgtgctcactcagccgtc

SEQ ID NO: 29 - DNA, 23 bp, Homo sapiens
aattttatgctgactcagcccca

SEQ ID NO: 30 - DNA, 24 bp, Homo sapiens
acgtttgatttccaccttggtccc

SEQ ID NO: 31 - DNA, 24 bp, Homo sapiens
acgtttgatctccagcttggtccc

SEQ ID NO: 32 - DNA, 24 bp, Homo sapiens
acgtttgatatccactttggtccc

SEQ ID NO: 33 - DNA, 24 bp, Homo sapiens
acgtttgatctccaccttggtccc

SEQ ID NO: 34 - DNA, 24 bp, Homo sapiens
acgtttaatctccagtcgtgtccc

SEQ ID NO: 35 - DNA, 23 bp, Homo sapiens
cagtctgtgttgacgcagccgcc

SEQ ID NO: 36 - DNA, 23 bp, Homo sapiens
cagtctgccctgactcagcctgc

SEQ ID NO: 37 - DNA, 23 bp, Homo sapiens
tcctatgtgctgactcagccacc

SEQ ID NO: 38 - DNA, 23 bp, Homo sapiens
tcttctgagctgactcaggaccc

SEQ ID NO: 39 - DNA, 23 bp, Homo sapiens
cacgttatactgactcaaccgcc

SEQ ID NO: 40 - DNA, 23 bp, Homo sapiens
caggctgtgctcactcagccgtc

SEQ ID NO: 41 - DNA, 23 bp, Homo sapiens
aattttatgctgactcagcccca

SEQ ID NO: 42 - protein, 120 aa, Homo sapiens
QVQLVQSGAEVKKPGASVKVSCKASGYTFTNYDINWVRQAPGQGLEWMGISAYTGNTNYAQKLQGRVTMTTDTSTSTAYMELRSLRSDDTAVYYCVRDYHDSNGYYYFDYWGQGTLVTVS

SEQ ID NO: 43 - protein, 111 aa, Homo sapiens
DIQMIQSPLSLPVIPGEPASMSCRSSRSLLHSNGNNYLEWYLQKPGQSPQLLIYLGSNRASGVPDRFSGSGSGTDFTLKISRVEAEDVGIYYCMQGLQLPWTFGGTKVIKR

SEQ ID NO: 44 - protein, 128 aa, Homo sapiens
QVQLVQSGGGVVQPGRSLRLSCAASGFTFRTYGMHWVRQAPGKGLEWVAVLWYDGTNKYYADSVKGRFAISRDNSNNTLYLQMNSLRAEDAAVYYCARDGSYYYDSSGYYYVGGFDYWGQGTLVTVSS

SEQ ID NO: 45 - protein, 108 aa, Homo sapiens
EIVLTQSPDFQSVTPKEKVTITCRASQNIGSSLHWYQQKPDQSPKLLIKSASQSFSGVPSRFSGSGSGTDFTLTINSLEAEDAATYYCHQSSSLPFTFGPGTKVDIKR

SEQ ID NO: 46 - protein, 128 aa, Homo sapiens
QVQLVESGGGVVQPGRSLRLSCSASGFTFSSYGIHWVRQAPGKGLEWVAVMWYAGSNEYYADSVKGRFTISRDNSKNTLYLQMNSLRAEDTAVYYCARDQGVLLRFGELRGYYGMDVWGQGTTVTVSS

SEQ ID NO: 47 - protein, 109 aa, Homo sapiens
EVVLTQSPGTLSLSLGERATLSCRASQSVSSSYLAWYQHKPGQAPRLLIYGTSSRATGIPDRFSGSGSGTNFTLTISRLEPEDFAVYYCQQYGSLPFTFGPGTKVDIKR

SEQ ID NO: 48 - protein, 137 aa, Homo sapiens
  human mature J chain
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 49 - protein, 137 aa, Artificial sequence
  Mutant form of human mature J chain with C134S mutation compared to wild type Mature form of human J chain (SEQ ID NO48)
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDASYPD

SEQ ID NO: 50 - protein, 112 aa, Artificial sequence
  Mutant form of human mature J chain with amino acids 113-137 deleted compared to wild type Mature form of human J chain (SEQ ID NO48)
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTA

SEQ ID NO: 51 - protein, 137 aa, Artificial sequence
  Mutant form of human mature J chain with C109S and C134S mutation compared to wild type mature form of human J chain (SEQ ID NO48)
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKSYTAVVPLVYGGETKMVETALTPDASYPD

(19) United States
(12) Patent Application Publication
(10) Pub. No.: US 2006/0062786 A1
(43) Pub. Date: Mar. 23, 2006

(54) Antibodies that immunospecifically bind to TRAIL receptors

(75) Inventors: Theodora W. Salcedo, East Syracuse, NY (US); Viktor Roschke, Rockville, MD (US); Steven M. Ruben, Brookeville, MD (US); Craig A. Rosen, Laytonsville, MD (US)

Correspondence Address: HUMAN GENOME SCIENCES INC;INTELLECTUAL PROPERTY DEPT., 14200 SHADY GROVE ROAD, ROCKVILLE, MD, 20850, US

(73) Assignee: Human Genome Sciences, Inc., Rockville, MD (US)

(21) Appl. No.: 11/221,281

(22) Filed: Sep. 8, 2005

Related U.S. Application Data
(63) Continuation-in-part of application No. 10/838,977, filed on May 5, 2004.
(63) Continuation-in-part of application No. 10/292,468, filed on Nov. 13, 2002, now Pat. No. 6,986,829.
(63) Continuation-in-part of application No. 09/986,149, filed on Nov. 7, 2001.
(63) Continuation-in-part of application No. 09/986,149, filed on Nov. 7, 2001.
(60) Provisional application No. 60/608,449, filed on Sep. 10, 2004.
(60) Provisional application No. 60/495,139, filed on Aug. 15, 2003.
(60) Provisional application No. 60/468,105, filed on May 6, 2003.
(60) Provisional application No. 60/403,376, filed on Aug. 15, 2002.
(60) Provisional application No. 60/377,973, filed on May 7, 2002.
(60) Provisional application No. 60/331,309, filed on Nov. 14, 2001.
(60) Provisional application No. 60/327,359, filed on Oct. 9, 2001.
(60) Provisional application No. 60/295,018, filed on Jun. 4, 2001.
(60) Provisional application No. 60/252,904, filed on Nov. 27, 2000.
(60) Provisional application No. 60/248,847, filed on Nov. 16, 2000.
(60) Provisional application No. 60/246,612, filed on Nov. 8, 2000.

(51) Int. Cl. C07H 21/02; C12P 21/06; A61K 39/395; C07K 16/30; C12N 5/06

(52) U.S. Cl. 424/143.1; 536/23.1; 530/388.22; 435/69.1; 435/334; 435/320.1

(57) ABSTRACT

The present invention relates to antibodies and related molecules that immunospecifically bind to TRAIL receptors. Such antibodies have uses, for example, in the prevention and treatment of cancers and other proliferative disorders. The invention also relates to nucleic acid molecules encoding anti-TRAIL receptor antibodies, vectors and host cells containing these nucleic acids, and methods for producing the same. The present invention relates to methods and compositions for preventing, detecting, diagnosing, treating or ameliorating a disease or disorder, especially cancer and other hyperproliferative disorders, comprising administering to an animal, preferably a human, an effective amount of one or more antibodies or fragments or variants thereof, or related molecules, that immunospecifically bind to TRAIL receptor.

### CROSS REFERENCE TO RELATED APPLICATIONS

This application claims benefit under 35 U.S.C. § 119(e) of U.S. Provisional Application No. 60/608,449, filed Sep. 10, 2004. This application is also a continuation-in-part of U.S. patent application Ser. No. 10/838,977, filed May 5, 2004, which claims benefit under 35 U.S.C. § 119(e) of U.S. Provisional Application Nos. 60/495,139, filed Aug. 15, 2003; and 60/468,105, filed May 6, 2003. This application is also a continuation-in-part of U.S. patent application Ser. No. 10/292,468, filed Nov. 13, 2002, which claims benefit under 35 U.S.C. § 119(e) of U.S. Provisional Application Nos. 60/403,376, filed Aug. 15, 2002; 60/377,973, filed May 7, 2002; and to 60/331,309, filed Nov. 14, 2001. This application is also a continuation-in-part of U.S. patent application Ser. No. 09/986,149, filed Nov. 7, 2001, which claims benefit under 35 U.S.C. § 119(e) of U.S. Provisional Application Nos. 60/327,359, filed Oct. 9, 2001; 60/295,018, filed Jun. 4, 2001; 60/252,904, filed Nov. 27, 2000; 60/248,847, filed Nov. 16, 2000; and 60/246,612, filed Nov. 8, 2000. U.S. patent application Ser. No. 10/292,468 is also a continuation-in-part of U.S. patent application Ser. No. 09/986,149. Each patent and patent application referenced above is hereby incorporated by reference herein in its entirety.

### FIELD OF THE INVENTION

The present invention relates to antibodies and related molecules that immunospecifically bind to TRAIL receptors. Such antibodies have uses, for example, in the prevention and treatment of cancers and other proliferative disorders. The invention also relates to nucleic acid molecules encoding anti-TRAIL receptor antibodies, vectors and host cells containing these nucleic acids, and methods for producing the same. The present invention relates to methods and compositions for preventing, detecting, diagnosing, treating or ameliorating a disease or disorder, especially cancer and other hyperproliferative disorders, comprising administering to an animal, preferably a human, an effective amount of one or more antibodies or fragments or variants thereof, or related molecules, that immunospecifically bind to TRAIL receptor.

### BACKGROUND OF THE INVENTION

Many biological actions, for instance, response to certain stimuli and natural biological processes, are controlled by factors, such as cytokines. Many cytokines act through receptors by engaging the receptor and producing an intra-cellular response.

For example, tumor necrosis factors (TNF) alpha and beta are cytokines which act through TNF receptors to regulate numerous biological processes, including protection against infection and induction of shock and inflammatory disease. The TNF molecules belong to the “TNF-ligand” superfamily, and act together with their receptors or counter-ligands, the “TNF-receptor” superfamily. So far, nine members of the TNF ligand superfamily have been identified and ten members of the TNF-receptor superfamily have been characterized.

Among the ligands there are included TNF-α, lymphotoxina (LT-α, also known as TNF-0), LT-0 (found in complex heterotrimer LT-α2-β), FasL, CD40L, CD27L, CD30L, 4-1BBL, OX40L and nerve growth factor (NGF). The superfamily of TNF receptors includes the p55TNF receptor, p75TNF receptor, TNF receptor-related protein, FAS antigen or APO-1, CD40, CD27, CD30, 4-1BB, OX40, low affinity p75 and NGF-receptor (Meager, A., Biologicals, 22:291-295 (1994)).

Many members of the TNF-ligand superfamily are expressed by activated T-cells, implying that they are necessary for T-cell interactions with other cell types which underlie cell ontogeny and functions. (Meager, A., supra).

Considerable insight into the essential functions of several members of the TNF receptor family has been gained from the identification and creation of mutants that abolish the expression of these proteins. For example, naturally occurring mutations in the FAS antigen and its ligand cause lymphoproliferative disease (Watanabe-Fukunaga, R., et al., Nature 356:314 (1992)), perhaps reflecting a failure of programmed cell death. Mutations of the CD40 ligand cause an X-linked immunodeficiency state characterized by high levels of immunoglobulin M and low levels of immunoglobulin G in plasma, indicating faulty T-cell-dependent B-cell activation (Allen, R. C. et al., Science 259:990 (1993)). Targeted mutations of the low affinity nerve growth factor receptor cause a disorder characterized by faulty sensory innovation of peripheral structures (Lee, K. F. et al., Cell 69:737 (1992)).

TNF and LT-α are capable of binding to two TNF receptors (the 55- and 75-kd TNF receptors). A large number of biological effects elicited by TNF and LT-α, acting through their receptors, include hemorrhagic necrosis of transplanted tumors, cytotoxicity, a role in endotoxic shock, inflammation, immunoregulation, proliferation and anti-viral responses, as well as protection against the deleterious effects of ionizing radiation. TNF and LT-α are involved in the pathogenesis of a wide range of diseases, including endotoxic shock, cerebral malaria, tumors, autoimmune disease, AIDS and graft-host rejection (Beutler, B. and Von Huffel, C., Science 264:667-668 (1994)). Mutations in the p55 Receptor cause increased susceptibility to microbial infection.

Moreover, an about 80 amino acid domain near the C-terminus of TNFR1 (p55) and Fas was reported as the “death domain,” which is responsible for transducing signals for programmed cell death (Tartaglia et al., Cell 74:845 (1993)).

Apoptosis, or programmed cell death, is a physiologic process essential to the normal development and homeostasis of multicellular organisms (H. Steller, Science 267, 1445-1449 (1995)). Derangements of apoptosis contribute to the pathogenesis of several human diseases including cancer, neurodegenerative disorders, and acquired immune deficiency syndrome (C. B. Thompson, Science 267, 1456-1462 (1995)). Recently, much attention has focused on the signal transduction and biological function of two cell surface death receptors, Fas/APO-1 and TNFR-1 (J. L. Cleveland, et al., Cell 81, 479-482 (1995); A. Fraser, et al., Cell 85, 781-784 (1996); S. Nagata, et al., Science 267, 1449-56 (1995)). Both are members of the TNF receptor family which also include TNFR-2, low affinity NGFR, CD40, and CD30, among others (C. A. Smith, et al., Science 248, 1019-23 (1990); M. Tewari, et al., in Modular Texts in Molecular and Cell Biology M. Purton, Heldin, Carl, Ed. (Chapman and Hall, London, 1995). While family members are defined by the presence of cysteine-rich repeats in their extracellular domains, Fas/APO-1 and TNFR-1 also share a region of intracellular homology, appropriately designated the “death domain”, which is distantly related to the Drosophila suicide gene, reaper (P. Golstein, et al., Cell 81, 185-6 (1995); K. White et al., Science 264, 677-83 (1994)). This shared death domain suggests that both receptors interact with a related set of signal transducing molecules that, until recently, remained unidentified. Activation of Fas/APO-1 recruits the death domain-containing adapter molecule FADD/MORT1 (A. M. Chinnaiyan, et al., Cell 81, 505-12 (1995); M. P. Boldin, et al., J. Biol Chem 270, 7795-8 (1995); F. C. Kischkel, et al., EMBO 14, 5579-5588 (1995)), which in turn binds and presumably activates FLICE/MACH1, a member of the ICE/CED-3 family of pro-apoptotic proteases (M. Muzio et al., Cell 85, 817-827 (1996); M. P. Boldin, et al., Cell 85, 803-815 (1996)). While the central role of Fas/APO-1 is to trigger cell death, TNFR-1 can signal an array of diverse biological activities-many of which stem from its ability to activate NF-kB (L. A. Tartaglia, et al., Immunol Today 13, 151-3 (1992)). Accordingly, TNFR-1 recruits the multivalent adapter molecule TRADD, which like FADD, also contains a death domain (H. Hsu, et al., Cell 81, 495-504 (1995); H. Hsu, et al., Cell 84, 299-308 (1996)). Through its associations with a number of signaling molecules including FADD, TRAF2, and REP, TRADD can signal both apoptosis and NF-kB activation (H. Hsu, et al., Cell 84, 299-308 (1996); H. Hsu, et al., Immunity 4, 387-396 (1996)).

One TNF-related apoptosis inducing ligand has been reported by several groups and has been ascribed the name Apoptosis Inducing Molecule I (AIM-I) (International Application No. WO 97/33899) and TNF-related apoptosis-inducing ligand or (TRAIL) (Wiley, S. R. et al., Immunity 3:673-682 (1995)). Pitti, R. M. et al., refer to the new molecule as Apo-2 ligand or (“Apo-2L”). For convenience, it will be referred to herein as TRAIL.

Unlike FAS ligand whose transcripts appear to be largely restricted to stimulated T-cells, significant levels of TRAIL are seen in many tissues, and it is constitutively transcribed by some cell lines. It has been shown that TRAIL acts independently from FAS ligand (Wiley, S. R., et al. (1995)), supra). Studies by Marsters, S. A. et al., have indicated that TRAIL activates apoptosis rapidly, within a time frame that is similar to death signalling by FAS/Apo-1L but much faster than TNF-induced apoptosis (Current Biology, 6:750-752 (1996)).

At least four TRAIL receptors have been identified, including TRAIL receptor 1 (TRAIL-R1, also referred to as TR4, and death receptor 4 (DR4), Pan et al., Science 276:111-3 (1997), International Patent Application Nos. WO 98/32856, WO00/67793, WO 99/37684, WO 2000/34355, WO 99/02653, SEQ ID NO:1); TRAIL receptor 2 (TRAIL-R2, also referred to as TR7, DR5, and KILLER, Pan et al., Science 277:815-8 (1997), Sheridan et al., Science 277:818-21 (1997), Chaudhury et al., Immunity 7:821-30 (1997), International Patent Application Nos. WO 98/46643, WO 99/09165, WO 99/11791, WO 98/41629, WO00/66156, and WO 98/35986, SEQ ID NO:3); TRAIL receptor 3 (TRAIL-R3, also referred to as TR5, decoy receptor 1 (DcR1) and TRID) (Degli-Esposti et al., J. Exp. Med. 186:1165-70 (1997), International Patent Application Nos. WO98/30693, WO0071150, WO 99/00423, EP867509, WO 98/58062, SEQ ID NO:2); and TRAIL Receptor 4 (TRAIL-R4, also referred to as TR10, DcR2, and TRUNDD, Pan et al., FEBS Lett. 424:41-5 (1998), Degli-Eposti et al., Immunity 7:813-20 (1997), International Patent Application Nos. WO 98/54202, WO00/73321, WO 2000/08155, WO 99/03992, WO 2000/34355 and WO99/0484, SEQ ID NO:4). TRAIL receptors 1 and 2 contain death domains in their cytoplasmic tails and the triggering of these receptors results in apoptosis. On the other hand TRAIL receptor 3 and 4 inhibit apoptosis induced by the cytotoxic ligand TRAIL in part because of their absent or truncated cytoplasmic death domains, respectively. Each of the publications and patents cited above is hereby incorporated by reference in their entireties.

The effects of TNF family ligands and TNF family receptors are varied and influence numerous functions, both normal and abnormal, in the biological processes of the mammalian system. There is a clear need, therefore, for identification and characterization of compositions, such as antibodies, that influence the biological activity of TNF receptors, both normally and in disease states. In particular, there is a need to isolate and characterize antibodies that modulate the biological activities of TRAIL receptors.

### SUMMARY OF THE INVENTION

The present invention encompasses antibodies (including molecules comprising, or alternatively consisting of, antibody fragments or variants thereof) that immunospecifically bind to a TRAIL receptor polypeptide or polypeptide fragment or variant of a TRAIL receptor. In particular, the invention encompasses antibodies (including molecules comprising, or alternatively consisting of, antibody fragments or variants thereof) that immunospecifically bind to a polypeptide or polypeptide fragment or variant of human TRAIL receptors such as those of SEQ ID NOS:1-4. In specific embodiments, the invention encompasses antibodies (including molecules comprising, or alternatively consisting of, antibody fragments or variants thereof) that immunospecifically bind to a polypeptide or polypeptide fragment or variant of human TR4 such as that of SEQ ID NO:1. In some embodiments, an antibody of the invention that immunospecifically bind to a TR4 polypeptide, also immunospecifically bind TR7 (e.g., SEQ ID NO:3), but not other proteins, including (TR5, and TR10 (SEQ ID NOS:2 and 4.)

The present invention relates to methods and compositions for preventing, treating or ameliorating a disease or disorder comprising administering to an animal, preferably a human, an effective amount of one or more antibodies or fragments or variants thereof, or related molecules, that immunospecifically bind to a TRAIL receptor or a fragment or variant thereof. In specific embodiments, the present invention relates to methods and compositions for preventing, treating or ameliorating a disease or disorder associated with TRAIL receptor function or TRAIL receptor ligand function or aberrant TRAIL receptor or TRAIL receptor ligand expression, comprising administering to an animal, preferably a human, an effective amount of one or more antibodies or fragments or variants thereof, or related molecules, that immunospecifically bind to a TRAIL receptor or a fragment or variant thereof. In highly preferred embodiments, the present invention relates to antibody-based methods and compositions for preventing, treating or ameliorating cancers and other hyperproliferative disorders (e.g., leukemia, carcinoma, and lymphoma). Other diseases and disorders which can be treated, prevented or ameliorated with the antibodies of the invention include, but are not limited to, neurodegenerative disorders (e.g., Parkinson's disease, Alzheimer's disease, and Huntington's disease), immune disorders (e.g., lupus, rheumatoid arthritis, multiple sclerosis, myasthenia gravis, Hashimoto's disease, and immunodeficiency syndrome), inflammatory disorders (e.g., asthma, allergic disorders, and rheumatoid arthritis), infectious diseases (e.g., AIDS, herpes viral infections, and other viral infections), proliferative disorders, and premalignant conditions (e.g., hyperplasias, metaplasias, and dysplasias).

The present invention also encompasses methods and compositions for detecting, diagnosing, or prognosing diseases or disorders comprising administering to an animal, preferably a human, an effective amount of one or more antibodies or fragments or variants thereof, or related molecules, that immunospecifically bind to TRAIL receptor or a fragment or variant thereof. In specific embodiments, the present invention also encompasses methods and compositions for detecting, diagnosing, or prognosing diseases or disorders associated with TRAIL receptor function or TRAIL receptor ligand function or aberrant TRAIL receptor or TRAIL receptor ligand expression, comprising administering to an animal, preferably a human, an effective amount of one or more antibodies or fragments or variants thereof, or related molecules, that immunospecifically bind to TRAIL receptor or a fragment or variant thereof. In highly preferred embodiments, the present invention relates to antibody-based methods and compositions for detecting, diagnosing, or prognosing cancers and other hyperproliferative disorders (e.g., leukemia, carcinoma, and lymphoma). Other diseases and disorders which can be detected, diagnosed or prognosed with the antibodies of the invention include, but are not limited to, neurodegenerative disorders (e.g., Parkinson's disease, Alzheimer's disease, and Huntington's disease), immune disorders (e.g., lupus, rheumatoid arthritis, multiple sclerosis, myasthenia gravis, Hashimoto's disease, and immunodeficiency syndrome), inflammatory disorders (e.g., asthma, allergic disorders, and rheumatoid arthritis), infectious diseases (e.g., AIDS, herpes viral infections, and other viral infections), proliferative disorders, and premalignant conditions (e.g., hyperplasias, metaplasias, and dysplasias).

Another embodiment of the present invention includes the use of the antibodies of the invention as a diagnostic tool to monitor the expression of TRAIL receptor expression on cells.

The present inventors have generated hybridoma cell lines that express antibodies that immunospecifically bind one or more TRAIL receptor polypeptides (e.g., SEQ ID NOs:1-4). Thus, the invention encompases these cell lines, listed in Table 1 below which were deposited with the American Type Culture Collection (“ATCC”) on the dates listed in Table 1 and given the ATCC Deposit Numbers identified in Table 1 The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

Further, the present invention encompasses the polynucleotides encoding the antibodies expressed by these cell lines, as well as the amino acid sequences encoding the antibodies expressed by these cell lines. Molecules comprising, or alternatively consisting of, fragments or variants of these antibodies (e.g., heavy chains, VH domains, VH CDRs, light chains, VL domains, or VL CDRs having an amino acid sequence of any one of those expressed by one or more cell lines referred to in Table 1), that immunospecifically bind to one or more TRAIL receptors or fragments or variants thereof are also encompassed by the invention, as are nucleic acid molecules that encode these antibodies and/or molecules. In highly preferred embodiments, the present invention encompasses antibodies, or fragments or variants thereof, that bind to the extracellular regions/domains of one or more TRAIL receptors or fragments and variants thereof.

The present invention also provides antibodies that bind one or more TRAIL receptor polypeptides which are coupled to a detectable label, such as an enzyme, a fluorescent label, a luminescent label, or a bioluminescent label. The present invention also provides antibodies that bind one or more TRAIL receptor polypeptides which are coupled to a therapeutic or cytotoxic agent. The present invention also provides antibodies that bind one or more TRAIL receptor polypeptides which are coupled to a radioactive material.

The present invention also provides antibodies that bind one or more TRAIL receptor polypeptides that act as either TRAIL receptor agonists or TRAIL receptor antagonists. In specific embodiments, the antibodies of the invention stimulate apoptosis of TRAIL receptor expressing cells. In other specific embodiments, the antibodies of the invention inhibit TRAIL binding to a TRAIL receptor. In other specific embodiments, the antibodies of the invention upregulate TRAIL receptor expression.

The present invention also provides antibodies that inhibit apoptosis of TRAIL receptor expressing cells. In other specific embodiments, the antibodies of the invention downregulate TRAIL receptor expression.

In further embodiments, the antibodies of the invention have a dissociation constant (K_{D}) of 10⁻⁷ M or less. In preferred embodiments, the antibodies of the invention have a dissociation constant (K_{D}) of 10⁻⁹ M or less.

The present invention further provides antibodies that stimulate apoptosis of TRAIL receptor expressing cells better than an equal concentration of TRAIL polypeptide stimulates apoptosis of TRAIL receptor expressing cells.

The present invention further provides antibodies that stimulate apoptosis of TRAIL receptor expressing cells equally well in the presence or absence of antibody cross-linking reagents; and/or stimulate apoptosis with equal or greater potency as an equal concentration of TRAIL in the absence of a cross-linking antibody or other cross-linking agent.

In further embodiments, antibodies of the invention have an off rate (k_{off}) of 10⁻³/sec or less. In preferred embodiments, antibodies of the invention have an off rate (k_{off) of} 10⁻⁴/sec or less. In other preferred embodiments, antibodies of the invention have an off rate (k_{off}) of 10⁻⁵/sec or less.

The present invention also provides for antibodies that preferentially bind one or more of the TRAIL receptors selected from the group of TR4, TR5, TR7, and TR10.

In certain embodiments, properties of the antibodies of the present invention, as detailed in the Examples below, make the antibodies better therapeutic agents than previously described TRAIL receptor binding antibodies.

The present invention also provides panels of antibodies (including molecules comprising, or alternatively consisting of, antibody fragments or variants) wherein the panel members correspond to one, two, three, four, five, ten, fifteen, twenty, or more different antibodies of the invention (e.g., whole antibodies, Fabs, F(ab′)₂ fragments, Fd fragments, disulfide-linked Fvs (sdFvs), anti-idiotypic (anti-Id) antibodies, and scFvs). The present invention further provides mixtures of antibodies, wherein the mixture corresponds to one, two, three, four, five, ten, fifteen, twenty, or more different antibodies of the invention (e.g., whole antibodies, Fabs, F(ab′)₂ fragments, Fd fragments, disulfide-linked Fvs (sdFvs), anti-idiotypic (anti-Id) antibodies, and scFvs)). The present invention also provides for compositions comprising, or alternatively consisting of, one, two, three, four, five, ten, fifteen, twenty, or more antibodies of the present invention (including molecules comprising, or alternatively consisting of, antibody fragments or variants thereof). A composition of the invention may comprise, or alternatively consist of, one, two, three, four, five, ten, fifteen, twenty, or more amino acid sequences of one or more antibodies or fragments or variants thereof. Alternatively, a composition of the invention may comprise, or alternatively consist of, nucleic acid molecules encoding one or more antibodies of the invention.

The present invention also provides for fusion proteins comprising an antibody (including molecules comprising, or alternatively consisting of, antibody fragments or variants thereof) of the invention, and a heterologous polypeptide (i.e., a polypeptide unrelated to an antibody or antibody domain). Nucleic acid molecules encoding these fusion proteins are also encompassed by the invention. A composition of the present invention may comprise, or alternatively consist of, one, two, three, four, five, ten, fifteen, twenty or more fusion proteins of the invention. Alternatively, a composition of the invention may comprise, or alternatively consist of, nucleic acid molecules encoding one, two, three, four, five, ten, fifteen, twenty or more fusion proteins of the invention.

The present invention also provides for a nucleic acid molecule(s), generally isolated, encoding an antibody (including molecules, such as scFvs, VH domains, or VL domains, that comprise, or alternatively consist of, an antibody fragment or variant thereof) of the invention. The present invention also provides a host cell transformed with a nucleic acid molecule of the invention and progeny thereof. The present invention also provides a method for the production of an antibody (including a molecule comprising, or alternatively consisting of, an antibody fragment or variant thereof) of the invention. The present invention further provides a method of expressing an antibody (including a molecule comprising, or alternatively consisting of, an antibody fragment or variant thereof) of the invention from a nucleic acid molecule. These and other aspects of the invention are described in further detail below.

### BRIEF DESCRIPTION OF THE FIGURES

FIG. 1: Flow cytometric staining of Hela, SW480 and HT1080 cells for TR4 (TRAIL-R1) expression using monoclonal antibody 7.3. Cells were incubated with 1 microgram/ml monoclonal antibody 7.3 for 45 minutes, washed and stained with anti-human IgG2-FITC detector antibody. Reactivity of the 7.3 monoclonal antibody with the cells is shown in the histogram with the dark line; isotype control staining is shown in the shaded histogram.

FIG. 2: Sensitivity of HeLa cells to killing mediated by TRAIL (A), monoclonal antibody 7.3 (B) or monoclonal antibody 7.12 (C). Sensitivity of HeLa cells to anti-TRAIL receptor monoclonal antibodies was tested in the presence of cycloheximide either with or without an equivalent amount of secondary goat anti-human Ig Fc specific antibody. Use of an equivalent amount of secondary goat anti-human Ig Fc specific antibody means that the secondary goat anti-human Ig Fc specific antibody concentration was equal to the concentration of the test antibodies, 7.3 and 7.12.

FIG. 3: Sensitivity of SW480 cells to killing mediated by TRAIL (A), monoclonal antibody 7.3 (B) monoclonal antibody 7.12 (C). Sensitivity of SW480 cells to monoclonal antibodies was tested in the presence of cycloheximide either with or without an equivalent amount of secondary goat anti-human Ig Fc specific antibody.

FIG. 4: Sensitivity of HT1080 cells to killing mediated by TRAIL (A), monoclonal antibody 7.3 (B) or monoclonal antibody 7.12 (C). Sensitivity of HT1080 cells to monoclonal antibodies was tested in the presence of cycloheximide either with or without an equivalent amount of secondary goat anti-human Ig Fc specific antibody.

FIG. 5: Sensitivity of HeLa, SW480 and HT1080 cells to killing mediated by anti-monoclonal antibody 7.12. Sensitivity of cells to monoclonal antibodies were tested in the absence of either cycloheximide or additional crosslinking with a secondary goat anti-human Ig Fc specific antibody.

FIG. 6: Sensitivity of HeLa and SW480 to TRAIL-Receptor mediated killing mediated induced by monoclonal antibody 7.12 in the presence of TOPOTECAN. A comparison is shown for sensitization of cells to TRAIL-R1 monoclonal antibody killing using either cycloheximide or topotecan.

FIG. 7: Sensitivity of SW480 cells to killing mediated by anti-TRAIL receptor monoclonal antibodies 7.3, 7.3.1, 7.3.2 or 7.3.3 (A) monoclonal antibodies 7.12, 7.12.1, 7.12.2, or 7.12.3 (B), monoclonal antibodies 7.10, 7.10.1, 7.10.2, or 7.10.3, or monoclonal antibodies 7.1.3, 7.2, 7.8, 8.3.1, or 8.3.2. Sensitivity of SW480 cells to monoclonal antibodies was tested in the presence of cycloheximide

FIG. 8: Effect of 7.12.2 treatment on tumor growth in Swiss nu/nu mice.

FIG. 9: Effect of 7.12.2 treatment on tumor growth in Swiss nu/nu mice-II.

### DETAILED DESCRIPTION OF THE INVENTION

Definitions

The term “antibody,” as used herein, refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody multimers and antibody fragments as well as variants (including derivatives) of antibodies, antibody multimers and antibody fragments. Examples of molecules which are described by the term “antibody” herein include, but are not limited to: single chain Fvs (scFvs), Fab fragments, Fab′ fragments, F(ab′)₂, disulfide linked Fvs (sdFvs), Fvs, and fragments comprising or alternatively consisting of, either a VL or a VH domain. The term “single chain Fv” or “scFv” as used herein refers to a polypeptide comprising a VL domain of antibody linked to a VH domain of an antibody. Antibodies that immunospecifically bind to a TRAIL receptor may have cross-reactivity with other antigens, e.g., another TRAIL Receptor. Preferably, antibodies that immunospecifically bind to a TRAIL receptor do not cross-react with other antigens (e.g., other TRAIL receptors or other members of the Tumor Necrosis Factor Receptor superfamily). Antibodies that immunospecifically bind to a TRAIL receptor can be identified, for example, by immunoassays or other techniques known to those of skill in the art, e.g., the immunoassays described in the Examples below.

Antibodies of the invention include, but are not limited to, monoclonal, multispecific, human or chimeric antibodies, single chain antibodies, Fab fragments, F(ab′) fragments, anti-idiotypic (anti-Id) antibodies (including, e.g., anti-id antibodies to antibodies of the invention), intracellularly-made antibodies (i.e., intrabodies), and epitope-binding fragments of any of the above. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. Preferably, an antibody of the invention comprises, or alternatively consists of, a VH domain, VH CDR, VL domain, or VL CDR having an amino acid sequence of any one of those referred to in Table 1, or a fragment or variant thereof. In a preferred embodiment, the immunoglobulin is an IgG1 isotype. In another preferred embodiment, the immunoglobulin is an IgG4 isotype. Immunoglobulins may have both a heavy and light chain. An array of IgG, IgE, IgM, IgD, IgA, and IgY heavy chains may be paired with a light chain of the kappa or lambda forms.

Antibodies of the invention may also include multimeric forms of antibodies. For example, antibodies of the invention may take the form of antibody dimers, trimers, or higher-order multimers of monomeric immunoglobulin molecules. Dimers of whole immunoglobulin molecules or of F(ab′)₂ fragments are tetravalent, whereas dimers of Fab fragments or scFv molecules are bivalent. Individual monomers within an antibody multimer may be identical or different, i.e., they may be heteromeric or homomeric antibody multimers. For example, individual antibodies within a multimer may have the same or different binding specificities. Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers, and other higher-order antibody multimers. Alternatively, antibody homodimers may be formed through chemical linkage techniques known in the art. For example, heterobifunctional crosslinking agents including, but not limited to, SMCC [succinimidyl 4-(maleimidomethyl)cyclohexane-1-carboxylate] and SATA [N-succinimidyl S-acethylthio-acetate] (available, for example, from Pierce Biotechnology, Inc. (Rockford, Ill.)) can be used to form antibody multimers. An exemplary protocol for the formation of antibody homodimers is given in Ghetie et al., Proceedings of the National Academy of Sciences USA (1997) 94:7509-7514, which is hereby incorporated by reference in its entirety. Antibody homodimers can be converted to Fab′2 homodimers through digestion with pepsin. Another way to form antibody homodimers is through the use of the autophilic T15 peptide described in Zhao and Kohler, The Journal of Immunology (2002) 25:396-404, which is hereby incorporated by reference in its entirety.

Alternatively, antibodies can be made to multimerize through recombinant DNA techniques. IgM and IgA naturally form antibody multimers through the interaction with the mature J chain polypeptide (e.g., SEQ ID NO:48). Non-IgA or non-IgM molecules, such as IgG molecules, can be engineered to contain the J chain interaction domain of IgA or IgM, thereby conferring the ability to form higher order multimers on the non-IgA or non-IgM molecules. (see, for example, Chintalacharuvu et al., (2001) Clinical Immunology 101:21-31. and Frigerio et al., (2000) Plant Physiology 123:1483-94., both of which are hereby incorporated by reference in their entireties.) IgA dimers are naturally secreted into the lumen of mucosa-lined organs. This secretion is mediated through interaction of the J chain with the polymeric IgA receptor (pIgR) on epithelial cells. If secretion of an IgA form of an antibody (or of an antibody engineered to contain a J chain interaction domain) is not desired, it can be greatly reduced by expressing the antibody molecule in association with a mutant J chain that does not interact well with pIgR (e.g., SEQ ID NOS:49-51; Johansen et al., The Journal of Immunology (2001) 167:5185-5192 which is hereby incorporated by reference in its entirety). SEQ ID NO:49 is a mutant form of a human mature J chain with C134S mutation compared to the mature form of human J chain (SEQ ID NO:48). SEQ ID NO:50 is a mutant form of a human mature J chain with amino acids 113-137 deleted compared to the mature form of human J chain (SEQ ID NO:48). SEQ ID NO:51 shows a mutant form of human mature J chain with C109S and C134S mutation compared to the mature form of human J chain (SEQ ID NO:48). Expression of an antibody with one of these mutant J chains will reduce its ability to bind to the polymeric IgA receptor on epithelial cells, thereby reducing transport of the antibody across the epithelial cell and its resultant secretion into the lumen of mucosa lined organs. ScFv dimers can also be formed through recombinant techniques known in the art; an example of the construction of scFv dimers is given in Goel et al., (2000) Cancer Research 60:6964-6971 which is hereby incorporated by reference in its entirety. Antibody multimers may be purified using any suitable method known in the art, including, but not limited to, size exclusion chromatography.

Unless otherwise defined in the specification, specific binding or immunospecific binding by an anti-TR4 antibody means that the anti-TR4 antibody binds TR4 but does not significantly bind to (i.e., cross react with) proteins other than TR4, such as other proteins in the same family of proteins). An antibody that binds TR4 protein and does not cross-react with other proteins is not necessarily an antibody that does not bind said other proteins in all conditions; rather, the TR4-specific antibody of the invention preferentially binds TR4 compared to its ability to bind said other proteins such that it will be suitable for use in at least one type of assay or treatment, i.e., give low background levels or result in no unreasonable adverse effects in treatment. It is well known that the portion of a protein bound by an antibody is known as the epitope. An epitope may either be linear (i.e., comprised of sequential amino acids residues in a protein sequences) or conformational (i.e., comprised of one or more amino acid residues that are not contiguous in the primary structure of the protein but that are brought together by the secondary, tertiary or quaternary structure of a protein). Given that TR4-specific antibodies bind to epitopes of TR4, an antibody that specifically binds TR4 may or may not bind fragments of TR4 and/or variants of TR4 (e.g., proteins that are at least 90% identical to TR4) depending on the presence or absence of the epitope bound by a given TR4-specific antibody in the TR4 fragment or variant. Likewise, TR4-specific antibodies of the invention may bind species orthologues of TR4 (including fragments thereof) depending on the presence or absence of the epitope recognized by the antibody in the orthologue. Additionally, TR4-specific antibodies of the invention may bind modified forms of TR4, for example, TR4 fusion proteins. In such a case when antibodies of the invention bind TR4 fusion proteins, the antibody must make binding contact with the TR4 moiety of the fusion protein in order for the binding to be specific. Antibodies that specifically bind to TR4 can be identified, for example, by immunoassays or other techniques known to those of skill in the art, e.g., the immunoassays described in the Examples below.

In some embodiments the present invention encompasses antibodies that immunospecifically or specifically bind both TR4 and TR7. Specific binding or immunospecific binding by an antibody that immunospecifically binds TR4 and TR7 means that the antibody binds TR4 and TR7 but does not significantly bind to (i.e., cross react with) proteins other than TR4 or TR7, such as other proteins in the same family of proteins). An antibody that binds TR4 and TR7 proteins and does not cross-react with other proteins is not necessarily an antibody that does not bind said other proteins in all conditions; rather, the antibody that immunospcifically or specifically binds both TR4 and TR7 preferentially binds TR4 and TR7 compared to its ability to bind said other proteins such that it will be suitable for use in at least one type of assay or treatment, i.e., give low background levels or result in no unreasonable adverse effects in treatment. It is well known that the portion of a protein bound by an antibody is known as the epitope. An epitope may either be linear (i.e., comprised of sequential amino acids residues in a protein sequences) or conformational (i.e., comprised of one or more amino acid residues that are not contiguous in the primary structure of the protein but that are brought together by the secondary, tertiary or quaternary structure of a protein). Given that antibodies that bind TR4 and TR7 bind to epitopes common to TR4 and TR7, an antibody that specifically binds TR4 and TR7 may or may not bind fragments of TR4, TR7 and/or variants of TR4 or TR7 (e.g., proteins that are at least 90% identical to TR4 or TR7, respectively) depending on the presence or absence of the epitope bound by a given antibody in the TR4 or TR7 fragment or variant. Likewise, antibodies of the invention that immunospecifically bind TR4 and TR7 may bind species orthologues of TR4 and/or TR7 (including fragments thereof) depending on the presence or absence of the epitope recognized by the antibody in the orthologues. Additionally, antibodies of the invention that immunospecifically bind TR4 and TR7 may bind modified forms of TR4 or TR7, for example, TR4 or TR7 fusion proteins. In such a case when antibodies of the invention bind fusion proteins, the antibody must make binding contact with the TR4 or TR7 moiety of the fusion protein in order for the binding to be specific. Antibodies that specifically bind to TR4 or TR7 can be identified, for example, by immunoassays or other techniques known to those of skill in the art, e.g., the immunoassays described in the Examples below.

The term “variant” as used herein refers to a polypeptide that possesses a similar or identical function as a TRAIL receptor polypeptide, a fragment of a TRAIL receptor polypeptide, an anti-TRAIL receptor antibody or antibody fragment thereof, but does not necessarily comprise a similar or identical amino acid sequence of a TRAIL receptor polypeptide, a fragment of a TRAIL receptor polypeptide, an anti-TRAIL receptor antibody or antibody fragment thereof, or possess a similar or identical structure of a TRAIL receptor polypeptide, a fragment of a TRAIL receptor polypeptide, an anti-TRAIL receptor antibody or antibody fragment thereof A variant having a similar amino acid refers to a polypeptide that satisfies at least one of the following: (a) a polypeptide comprising, or alternatively consisting of, an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a TRAIL receptor polypeptide, a fragment of, an anti-TRAIL receptor antibody or antibody fragment thereof (including a VH domain, VHCDR, VL domain, or VLCDR having an amino acid sequence of any one of those expressed by one or more cell lines referred to in Table 1) described herein; (b) a polypeptide encoded by a nucleotide sequence, the complementary sequence of which hybridizes under stringent conditions to a nucleotide sequence encoding a TRAIL receptor polypeptide (e.g., SEQ ID NO:1-4), a fragment of a TRAIL receptor polypeptide, an anti-TRAIL receptor antibody or antibody fragment thereof (including a VH domain, VHCDR, VL domain, or VLCDR having an amino acid sequence of any one of those referred to in Table 1), described herein, of at least 5 amino acid residues, at least 10 amino acid residues, at least 15 amino acid residues, at least 20 amino acid residues, at least 25 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99%, identical to the nucleotide sequence encoding a TRAIL receptor polypeptide, a fragment of a TRAIL receptor polypeptide, an anti-TRAIL receptor antibody or antibody fragment thereof (including a VH domain, VBCDR, VL domain, or VLCDR having an amino acid sequence of any one of those expressed by one or more cell lines referred to in Table 1), described herein. A polypeptide with similar structure to a TRAIL receptor polypeptide, a fragment of a TRAIL receptor polypeptide, an anti-TRAIL receptor antibody or antibody fragment thereof, described herein refers to a polypeptide that has a similar secondary, tertiary or quaternary structure of a TRAIL receptor polypeptide, a fragment of a TRAIL receptor polypeptide, an anti-TRAIL receptor antibody, or antibody fragment thereof described herein. The structure of a polypeptide can determined by methods known to those skilled in the art, including but not limited to, X-ray crystallography, nuclear magnetic resonance, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can be accomplished using a mathematical algorithm known to those of skill in the art. An example of a mathematical algorithm for comparing two sequences is the algorithm of Karlin and Altschul Proc. Natl. Acad. Sci. USA 87:2264-2268(1990), modified as in Karlin and Altschul Proc. Natl. Acad. Sci. USA 90:5873-5877(1993). The BLASTn and BLASTx programs of Altschul, et al. J. Mol. Biol. 215:403-410(1990) have incorporated such an algorithm. BLAST nucleotide searches can be performed with the BLASTn program (score=100, wordlength=12) to obtain nucleotide sequences homologous to a nucleic acid molecules of the invention. BLAST protein searches can be performed with the BLASTx program (score=50, wordlength=3) to obtain amino acid sequences homologous to a protein molecules of the invention. To obtain gapped alignments for comparison purposes, Gapped BLAST can be utilized as described in Altschul et al. Nucleic Acids Res. 25:3589-3402(1997). Alternatively, PSI-BLAST can be used to perform an iterated search which detects distant relationships between molecules (Id.). When utilizing BLAST, Gapped BLAST, and PSI-BLAST programs, the default parameters of the respective programs (e.g., BLASTx and BLASTn) can be used. (See http://www.ncbi.nlm.nih.gov.)

Another example of a mathematical algorithm utilized for the comparison of sequences is the algorithm of Myers and Miller, CABIOS (1989). The ALIGN program (version 2.0) which is part of the GCG sequence alignment software package has incorporated such an alogrithm. Other algorithms for sequence analysis known in the art include ADVANCE and ADAM as described in Torellis and Robotti Comput. Appl. Biosci., 10 :3-5(1994); and FASTA described in Pearson and Lipman Proc. Nail. Acad. Sci. 85:2444-8(1988). Within FASTA, ktup is a control option that sets the sensitivity and speed of the search.

The term “derivative” as used herein, refers to a variant polypeptide of the invention that comprises, or alternatively consists of, an amino acid sequence of a TRAIL receptor polypeptide, a fragment of a TRAIL receptor polypeptide, or an antibody of the invention that immunospecifically binds to a TRAIL receptor polypeptide, which has been altered by the introduction of amino acid residue substitutions, deletions or additions. The term “derivative” as used herein also refers to a TRAIL receptor polypeptide, a fragment of a TRAIL receptor polypeptide, an antibody that immunospecifically binds to a TRAIL receptor polypeptide which has been modified, e.g., by the covalent attachment of any type of molecule to the polypeptide. For example, but not by way of limitation, a TRAIL receptor polypeptide, a fragment of a TRAIL receptor polypeptide, or an anti-TRAIL receptor antibody, may be modified, e.g., by glycosylation, acetylation, pegylation, phosphorylation, amidation, derivatization by known protecting/blocking groups, proteolytic cleavage, linkage to a cellular ligand or other protein, etc. A derivative of a TRAIL receptor polypeptide, a fragment of a TRAIL receptor polypeptide, or an anti-TRAIL receptor antibody, may be modified by chemical modifications using techniques known to those of skill in the art, including, but not limited to, specific chemical cleavage, acetylation, formylation, metabolic synthesis of tunicamycin, etc. Further, a derivative of a TRAIL receptor polypeptide, a fragment of a TRAIL receptor polypeptide, or an anti-TRAIL receptor antibody, may contain one or more non-classical amino acids. A polypeptide derivative possesses a similar or identical function as a TRAIL receptor polypeptide, a fragment of a TRAIL receptor polypeptide, or an anti-TRAIL receptor antibody, described herein.

The term “epitopes” as used herein refers to portions of TRAIL receptor having antigenic or immunogenic activity in an animal, preferably a mammal. An epitope having immunogenic activity is a portion of TRAIL receptor that elicits an antibody response in an animal. An epitope having antigenic activity is a portion of TRAIL receptor to which an antibody immunospecifically binds as determined by any method known in the art, for example, by the immunoassays described herein. Antigenic epitopes need not necessarily be immunogenic.

The term “fragment” as used herein refers to a polypeptide comprising an amino acid sequence of at least 5 amino acid residues, at least 10 amino acid residues, at least 15 amino acid residues, at least 20 amino acid residues, at least 25 amino acid residues, at least 30 amino acid residues, at least 35 amino acid residues, at least 40 amino acid residues, at least 45 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues, of the amino acid sequence of a TRAIL receptor, or an anti-TRAIL receptor antibody (including molecules such as scFv's, that comprise, or alternatively consist of, antibody fragments or variants thereof) that immunospecifically binds to TRAIL receptor.

The term “fusion protein” as used herein refers to a polypeptide that comprises, or alternatively consists of, an amino acid sequence of an anti-TRAIL receptor antibody of the invention and an amino acid sequence of a heterologous polypeptide (i.e., a polypeptide unrelated to an antibody or antibody domain).

The term “host cell” as used herein refers to the particular subject cell transfected with a nucleic acid molecule and the progeny or potential progeny of such a cell. Progeny may not be identical to the parent cell transfected with the nucleic acid molecule due to mutations or environmental influences that may occur in succeeding generations or integration of the nucleic acid molecule into the host cell genome.

By “isolated antibody” is intended an antibody removed from its native environment. Thus, an antibody produced and/or contained within a recombinant host cell is considered isolated for purposes of the present invention.

Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact IgG antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the heavy and the light chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547 1553 (1992). In addition, bispecific antibodies may be formed as “diabodies” (Holliger et al. “‘Diabodies’: small bivalent and bispecific antibody fragments” PNAS USA 90:6444-448 (1993)) or “Janusins” (Traunecker et al. “Bispecific single chain molecules (Janusins) target cytotoxic lymphocytes on HIV infected cells” EMBO J 10:3655-3659 (1991) and Traunecker et al. “Janusin: new molecular design for bispecific reagents” Int J Cancer Suppl 7:51-52 (1992)).

Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

Anti-TRAIL Receptor Antibodies

The present invention is directed to fully human antibodies, generally isolated, that immunospecifically bind one or more TRAIL receptor polypeptides. Essentially, XenoMouse lines of mice from Abgenix, Inc. (Fremont, Calif.) expressing human antibodies were immunized with TRAIL receptor polypeptides, lymphatic cells (such as B-cells) were recovered from the mice that had high titers of anti-TRAIL receptor antibodies, and such recovered cells were fused with a myeloid-type cell line to prepare immortal hybridoma cell lines. Hybridoma cell lines were screened to select and identify hybridoma cell lines that produced antibodies specific to the immunogen. We utilized these techniques in accordance with the present invention for the preparation of antibodies specific to TRAIL receptor polypeptides. Herein, we describe the production of multiple hybridoma cell lines that produce antibodies specific to TRAIL receptor polypeptides. Further, we provide a characterization of the antibodies produced by such cell lines.

The antibodies derived from hybridoma cell lines discussed herein are listed in Table 1. Preferred antibodies of the invention include, antibodies expressed by the following cell lines: 1.2, 1.3, 7.1, 7.3, 7.8, 7.10, 7.12, and 8.3 (including the antibodies expressed by each of the subclones of these lines). XenoMouse strains of mice from Abgenix, Inc. express human kappa light chains with either human IgG1, IgG2, or IgG4. The IgG2 expressing strain was used to make the cell lines and antibodies of the present invention, thus each of the antibodies produced by cell lines are fully human IgG2 heavy chains with human kappa light chains. These hybridoma cell lines were deposited with the American Type Culture Collection (“ATCC”) on the date listed in Table 1, and given ATCC Deposit Numbers listed in Table 1. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

As described in Example 1, hybridoma cell lines that have a numeric designation that contains one period indicates a primary hybridoma isolate. The number preceding the period indicates the fusion panel a hybridoma came from and the number after the period designates the primary hybridoma isolate number. By “primary hybridoma isolate” is meant a hybridoma obtained by fusing spleen cells of immunized mice with a fusion partner, plating the cells at limiting dilution in 96 well plates, and selecting a hybridoma cell line, that by visual inspection appeared to have only a single colony, i.e., that appeared to have grown up from a single cell. Such a hybridoma cell line is most likely a monoclonal cell line. Primary hybridoma isolates were also subcloned. In a subcloning procedure, cells corresponding to a single primary hybridoma isolate are plated out at limiting dilution, and hybridoma subclones, that by visual inspection appeared to have only a single colony, i.e., that appeared to have grown up from a single cell, are selected. In this application, hybridoma subclones have designations containing two periods. As above, the number preceding the first period indicates which fusion panel a hybridoma came from; the number immediately after the first period designates the primary hybridoma isolate number; and the number following the second period indicates the number of a particular subclone derived from the primary hybridoma isolate with the designation indicated by the number immediately following the first period. Subcloned cell lines are monoclonal and are typically more stable with respect to antibody expression.

The following hybridoma cell lines deposited at the American Type Culture Collection (ATCC) contain equal proportions of three subclones of a particular hybridoma isolate. Hybridomas 7.3.1, 7.3.2, and 7.3.3 were collectively deposited at the ATCC on Nov. 16, 2000 and given ATCC Deposit Number PTA-2687. Hybridomas 7.8.1, 7.8.2, and 7.8.3 were collectively deposited at the ATCC on Nov. 27, 2000 and given ATCC Deposit Number PTA-2730. Hybridomas 7.10.1, 7.10.2, and 7.10.3 were collectively deposited at the ATCC on Nov. 27, 2000 and given ATCC Deposit Number PTA-2729. Hybridomas 7.12.1, 7.12.2, and 7.12.3 were collectively deposited at the ATCC on Nov. 27, 2000 and given ATCC Deposit Number PTA-2728. Hybridomas 8.3.1 and 8.3.2 were collectively deposited at the ATCC on Nov. 27, 2000 and given ATCC Deposit Number PTA-2731.

Individual hybridoma 7.1.3 was deposited at the ATCC on Mar. 2, 2001 and given ATCC Deposit Number PTA-3149. Individual hybridoma 7.3.3 was deposited at the ATCC on May 11, 2001 and given ATCC Deposit Number PTA-3369. Individual hybridoma 7.12.2 was deposited at the ATCC on May 11, 2001 and given ATCC Deposit Number PTA-3368.

The ATCC is located at 10801 University Boulevard, Manassas, Va. 20110-2209, USA. Each of the ATCC deposits described herein was made pursuant to the terms of the Budapest Treaty on the international recognition of the deposit of microorganisms for purposes of patent procedure. The ATCC Deposit Numbers and the hybridoma designations are also presented in Table 1.

TABLE 1
Hybridoma Cell Lines Expressing anti-TRAIL Receptor Antibodies

| Primary | | VH | VL | AAs | AAs | AAs | AAs | AAs | AAs | | |
|---|---|---|---|---|---|---|---|---|---|---|---|
| Hybridoma | | SEQ ID | SEQ ID | of VH | of VH | of VH | of VL | of VL | of VL | ATCC Deposit | ATCC Deposit |
| Isolate | Subclone | NO: | NO: | CDR1 | CDR2 | CDR3 | CDR1 | CDR2 | CDR3 | Number | Date |
| 1.2 | 1.2.1 | | | | | | | | | | |
| | 1.2.2 | | | | | | | | | | |
| | 1.2.3 | | | | | | | | | | |
| 1.3 | 1.3.1 | | | | | | | | | | |
| | 1.3.2 | | | | | | | | | | |
| | 1.3.3 | | | | | | | | | | |
| 3.1 | 3.1.1 | | | | | | | | | | |
| | 3.1.2 | | | | | | | | | | |
| | 3.1.3 | | | | | | | | | | |
| | 3.1.4 | | | | | | | | | | |
| | 3.1.5 | | | | | | | | | | |
| | 3.1.6 | | | | | | | | | | |
| 3.3 | 3.3.1 | | | | | | | | | | |
| 3.4 | 3.4.2 | | | | | | | | | | |
| | 3.4.4 | | | | | | | | | | |
| 7.1 | 7.1.3 | 42 | 43 | 31-35 | 50-65 | 98-110 | 24-39 | 55-61 | 94-102 | PTA-3149 | Mar. 02, 2001 |
| 7.2 | | | | | | | | | | | |
| 7.3 | 7.3.1 | | | | | | | | | PTA-2687 | Nov. 16, 2000 |
| | 7.3.2 | | | | | | | | | | Nov. 16, 2000 |
| | 7.3.3 | | | | | | | | | | Nov. 16, 2000 |
| 7.3 | 7.3.3 | 44 | 45 | 31-35 | 50-66 | 99-117 | 24-34 | 50-56 | 89-97 | PTA-3369 | May 11, 2001 |
| 7.4 | | | | | | | | | | | |
| 7.5 | | | | | | | | | | | |
| 7.6 | | | | | | | | | | | |
| 7.7 | | | | | | | | | | | |
| 7.8 | 7.8.1 | | | | | | | | | PTA-2730 | Nov. 27, 2000 |
| | 7.8.2 | | | | | | | | | | Nov. 27, 2000 |
| | 7.8.3 | | | | | | | | | | Nov. 27, 2000 |
| 7.9 | | | | | | | | | | | |
| 7.10 | 7.10.1 | | | | | | | | | PTA-2729 | Nov. 27, 2000 |
| | 7.10.2 | | | | | | | | | | Nov. 27, 2000 |
| | 7.10.3 | | | | | | | | | | Nov. 27, 2000 |
| 7.11 | | | | | | | | | | | |
| 7.12 | 7.12.1 | | | | | | | | | PTA-2728 | Nov. 27, 2000 |
| | 7.12.2 | | | | | | | | | | Nov. 27, 2000 |
| | 7.12.3 | | | | | | | | | | Nov. 27, 2000 |
| 7.12 | 7.12.2 | 46 | 47 | 31-35 | 50-66 | 99-117 | 24-35 | 51-57 | 90-98 | PTA-3368 | May 11, 2001 |
| 7.13 | | | | | | | | | | | |
| 8.2 | | | | | | | | | | | |
| 8.3 | 8.3.1 | | | | | | | | | PTA-2731 | Nov. 27, 2000 |
| | 8.3.2 | | | | | | | | | | Nov. 27, 2000 |
| 8.4 | | | | | | | | | | | |
| 8.5 | | | | | | | | | | | |
| 8.6 | | | | | | | | | | | |
| 9.1 | | | | | | | | | | | |
| 9.2 | | | | | | | | | | | |
| 9.3 | | | | | | | | | | | |
| 9.4 | | | | | | | | | | | |
| 9.5 | | | | | | | | | | | |
| 9.6 | | | | | | | | | | | |
| 9.7 | | | | | | | | | | | |
| 9.8 | | | | | | | | | | | |
| 9.9 | | | | | | | | | | | |
| 9.10 | | | | | | | | | | | |
| 9.11 | | | | | | | | | | | |
| 9.12 | | | | | | | | | | | |
| 9.13 | | | | | | | | | | | |
| 9.14 | | | | | | | | | | | |
| 9.15 | | | | | | | | | | | |
| 9.16 | | | | | | | | | | | |
| XF2-1A12 | | | | | | | | | | | |
| XF2-1G3 | | | | | | | | | | | |
| XF2-1G5 | | | | | | | | | | | |
| XF2-2D2 | | | | | | | | | | | |
| XF2-2H1 | | | | | | | | | | | |
| XF2-4C2 | | | | | | | | | | | |
| XF2-4F7 | | | | | | | | | | | |
| XF2-4G8 | | | | | | | | | | | |
| XF2-4H5 | | | | | | | | | | | |
| XF2-4H11 | | | | | | | | | | | |
| XF2-18A10 | | | | | | | | | | | |
| XF2-19C10 | | | | | | | | | | | |
| XF2-23H7 | | | | | | | | | | | |

If an antibody expressed by one (or two) of the subclones has a property that is distinct from the remaining subclone(s) of a given primary hybridoma isolate, a cell line expressing the antibody with that property can be retrieved from the pooled ATCC deposit using methods that are routine in the art. Briefly, retrieval of such a clone would require plating cells of the ATCC deposit at limiting dilution, growing the cells in culture, selecting monoclonal cell lines, and testing the antibodies expressed by the monoclonal cell lines for the presence or absence of the property using methods that are routine in the art. By way of non-limiting example, if one of the subclones expressed an antibody with a superior affinity for a TRAIL receptor polypeptide of the invention, one would test the affinities of the antibodies expressed by the monoclonal cell lines derived from the ATCC deposit. A monoclonal cell line that expressed an antibody with an affinity matching or resembling the desired affinity (making allowances for experimental variations in determinations of affinity) would be equivalent to the specific subclone from the ATCC deposit sought after.

As a matter of convenience, reference to an antibody herein by the primary hybridoma designation references not only to the primary hybridoma isolate but also each of its subclones deposited at the ATCC. For example reference to hybridoma cell line 7.3, references hybridoma cell lines 7.3, 7.3.1, 7.3.2, and 7.3.3. The only exceptions to this policy are the recitation of hybridoma designations in the Figures, Figure Legends, Examples, and claims. In those portions of the application, reference to a particular hybridoma designation references only the hybridoma defined by that designation. Additionally, the antibody secreted by a hybridoma cell lines has the same designation as the cell line itself Thus the term 7.3 may also reference the antibody expressed by hybridoma cell lines 7.3, 7.3.1, 7.3.2, and 7.3.3.

The present invention encompasses antibodies (including molecules comprising, or alternatively consisting of, antibody fragments or variants thereof) that immunospecifically bind to a TRAIL receptor polypeptide or a fragment, variant, or fusion protein thereof A TRAIL receptor polypeptide includes, but is not limited to, TR4 (SEQ ID NO:1) or the polypeptide encoded by the cDNA in clone HCUDS60 contained in ATCC Deposit 97853 deposited Jan. 21, 1997; TR5 (SEQ ID NO:2) or the polypeptide encoded by the cDNA in clone HPRCB54 contained in ATCC Deposit 97798 deposited Nov. 20, 1996; TR7 (SEQ ID NO:3) or the polypeptide encoded by the cDNA in clone HLYBX88 contained in ATCC Deposit 97920 deposited Mar. 7, 1997, and/or TR10 (SEQ ID NO:4) or the polypeptide encoded by the cDNA in clone HKABO35 contained in ATCC Deposit 209040 deposited May 15, 1997. In some embodiments, antibodies of the present invention may immunospecifically bind to both TR4 as described above and to TR7 (SEQ ID NO:3) or the polypeptide encoded by the cDNA in clone HLYBX88 contained in ATCC Deposit 97920 deposited Mar. 7, 1997. TRAIL receptors may be produced through recombinant expression of nucleic acids encoding the polypeptides of SEQ ID NOS:1-4, (e.g., the cDNAs in the ATCC Deposit Numbers 97853, 97798, 97920, or 209040).

In one embodiment, the antibodies of the invention preferentially bind TR4 (SEQ ID NO:1), or fragments, variants, or fusion proteins thereof (e.g., the extracellular region of TR4 fused to an Fc domain) relative to their ability to bind TR5, TR7, or TR10 (SEQ ID NOS:2-4) or fragments, variants, or fusion proteins thereof. In another preferred embodiment, antibodies of the invention preferentially bind TR7, fragments, variants, or fusion proteins thereof (e.g., the extracellular region of TR7 fused to an Fc domain) relative to their ability to bind TR4, TR5, or TR10 (SEQ ID NOS:1, 2, and 4) or fragments, variants, or fusion proteins thereof. In other preferred embodiments, the antibodies of the invention preferentially bind to TR4 and TR7 (SEQ ID NOS:1 and 3), or fragments and variants thereof relative to their ability to bind TR5 or TR10 (SEQ ID NOS:2 and 4) or fragments, variants, or fusion proteins thereof. In other preferred embodiments, the antibodies of the invention preferentially bind to TR5 and TR10 (SEQ ID NOS:2 and 4), or fragments and variants thereof relative to their ability to bind TR4 or TR7 (SEQ ID NOS:1 and 3) or fragments, variants, or fusion proteins thereof. In other preferred embodiments, the antibodies of the invention bind TR4, TR5, TR7 and TR10 (SEQ ID NOS:1-4). In another embodiment, antibodies of the invention preferentially bind TR5 (SEQ ID NO:2), or fragments and variants thereof relative to their ability to bind TR4, TR7 or TR10 (SEQ ID NOS:1, 2, and 3). In another embodiment, antibodies of the invention preferentially bind TR10 (SEQ ID NO:4), or fragments and variants thereof relative to their ability to bind TR4, TR5, or TR7 (SEQ ID NOS:1-3). An antibody's ability to preferentially bind one antigen compared to another antigen may be determined using any method known in the art.

TRAIL Receptor Polypeptides

TR4

In certain embodiments of the present invention, the antibodies of the present invention bind TR4 polypeptide, or fragments or variants thereof. The following section describes the TR4 polypeptides, fragments and variants that may be bound by the antibodies of the invention in more detail. The TR4 polypeptides, fragments and variants which may be bound by the antibodies of the invention are also described in International Publication Numbers, for example, WO98/32856 and WO00/67793 which are herein incorporated by reference in their entireties.

In certain embodiments, the antibodies of the present invention immunospecifically bind TR4 polypeptide. An antibody that immunospecifically binds TR4 may, in some embodiments, bind fragments, variants (including species orthologs of TR4), multimers or modified forms of TR4. For example, an antibody immunospecific for TR4 may bind the TR4 moiety of a fusion protein comprising all or a portion of TR4.

TR4 proteins may be found as monomers or multimers (i.e., dimers, trimers, tetramers, and higher multimers). Accordingly, the present invention relates to antibodies that bind TR4 proteins found as monomers or as part of multimers. In specific embodiments, antibodies of the invention bind TR4 monomers, dimers, trimers or tetramers. In additional embodiments, antibodies of the invention bind at least dimers, at least trimers, or at least tetramers containing one or more TR4 polypeptides.

Antibodies of the invention may bind TR4 homomers or heteromers. As used herein, the term homomer, refers to a multimer containing only TR4 proteins of the invention (including TR4 fragments, variants, and fusion proteins, as described herein). These homomers may contain TR4 proteins having identical or different polypeptide sequences. In a specific embodiment, a homomer of the invention is a multimer containing only TR4 proteins having an identical polypeptide sequence. In another specific embodiment, antibodies of the invention bind TR4 homomers containing TR4 proteins having different polypeptide sequences. In specific embodiments, antibodies of the invention bind a TR4 homodimer (e.g., containing TR4 proteins having identical or different polypeptide sequences) or a homotrimer (e.g., containing TR4 proteins having identical or different polypeptide sequences). In additional embodiments, antibodies of the invention bind at least a homodimer, at least a homotrimer, or at least a homotetramer of TR4.

As used herein, the term heteromer refers to a multimer containing heterologous proteins (i.e., proteins containing polypeptide sequences that do not correspond to a polypeptide sequences encoded by the TR4 gene) in addition to the TR4 proteins of the invention. In a specific embodiment, antibodies of the invention bind a heterodimer, a heterotrimer, or a heterotetramer. In additional embodiments, the antibodies of the invention bind at least a homodimer, at least a homotrimer, or at least a homotetramer containing one or more TR4 polypeptides.

Multimers bound by one or more antibodies of the invention may be the result of hydrophobic, hydrophilic, ionic and/or covalent associations and/or may be indirectly linked, by for example, liposome formation. Thus, in one embodiment, multimers bound by one or more antibodies of the invention, such as, for example, homodimers or homotrimers, are formed when TR4 proteins contact one another in solution. In another embodiment, heteromultimers bound by one or more antibodies of the invention, such as, for example, heterotrimers or heterotetramers, are formed when proteins of the invention contact antibodies to the TR4 polypeptides (including antibodies to the heterologous polypeptide sequence in a fusion protein) in solution. In other embodiments, multimers bound by one or more antibodies of the invention are formed by covalent associations with and/or between the TR4 proteins of the invention. Such covalent associations may involve one or more amino acid residues contained in the polypeptide sequence of the protein (e.g., the polypeptide sequence recited in SEQ ID NO:1 or the polypeptide encoded by the deposited cDNA clone of ATCC Deposit 97853). In one instance, the covalent associations are cross-linking between cysteine residues located within the polypeptide sequences of the proteins which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a TR4 fusion protein. In one example, covalent associations are between the heterologous sequence contained in a fusion protein (see, e.g., U.S. Pat. No. 5,478,925). In a specific example, the covalent associations are between the heterologous sequence contained in a TR4-Fc fusion protein (as described herein). In another specific example, covalent associations of fusion proteins are between heterologous polypeptide sequences from another TNF family ligand/receptor member that is capable of forming covalently associated multimers, such as for example, osteoprotegerin (see, e.g., International Publication No. WO 98/49305, the contents of which are herein incorporated by reference in its entirety).

The multimers that may be bound by one or more antibodies of the invention may be generated using chemical techniques known in the art. For example, proteins desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers that may be bound by one or more antibodies of the invention may be generated using techniques known in the art to form one or more inter-molecule cross-links between the cysteine residues located within the polypeptide sequence of the proteins desired to be contained in the multimer (see, e.g., U.S. Pat. No. 5,478,925, which is herein incorporated by reference in its entirety). Further, proteins that may be bound by one or more antibodies of the invention may be routinely modified by the addition of cysteine or biotin to the C terminus or N-terminus of the polypeptide sequence of the protein and techniques known in the art may be applied to generate multimers containing one or more of these modified proteins (see, e.g., U.S. Pat. No. 5,478,925, which is herein incorporated by reference in its entirety). Additionally, techniques known in the art may be applied to generate liposomes containing the protein components desired to be contained in the multimer that may be bound by one or more antibodies of the invention (see, e.g., U.S. Pat. No. 5,478,925, which is herein incorporated by reference in its entirety).

Alternatively, multimers that may be bound by one or more antibodies of the invention may be generated using genetic engineering techniques known in the art. In one embodiment, proteins contained in multimers that may be bound by one or more antibodies of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer that may be bound by one or more antibodies of the invention are generated by ligating a polynucleotide sequence encoding a TR4 polypeptide to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant TR4 polypeptides which contain a transmembrane domain and which can be incorporated by membrane reconstitution techniques into liposomes (see, e.g., U.S. Pat. No. 5,478,925, which is herein incorporated by reference in its entirety). In another embodiment, two or more TR4 polypeptides are joined through synthetic linkers (e.g., peptide, carbohydrate or soluble polymer linkers). Examples include those peptide linkers described in U.S. Pat. No. 5,073,627 (hereby incorporated by reference). Proteins comprising multiple TR4 polypeptides separated by peptide linkers may be produced using conventional recombinant DNA technology. In specific embodiments, antibodies of the invention bind proteins comprising multiple TR4 polypeptides separated by peptide linkers.

Another method for preparing multimer TR4 polypeptides involves use of TR4 polypeptides fused to a leucine zipper or isoleucine polypeptide sequence. Leucine zipper domains and isoleucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric TR4 proteins are those described in PCT application WO 94/10308, hereby incorporated by reference. Recombinant fusion proteins comprising a soluble TR4 polypeptide fused to a peptide that dimerizes or trimerizes in solution are expressed in suitable host cells, and the resulting soluble multimeric TR4 is recovered from the culture supernatant using techniques known in the art. In specific embodiments, antibodies of the invention bind TR4-leucine zipper fusion protein monomers and/or TR4-leucine zipper fusion protein multimers.

Certain members of the TNF family of proteins are believed to exist in trimeric form (Beutler and Huffel, Science 264:667, 1994; Banner et al., Cell 73:431, 1993). Thus, trimeric TR4 may offer the advantage of enhanced biological activity. Preferred leucine zipper moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. In specific embodiments, antibodies of the invention bind TR4-leucine zipper fusion protein trimers.

Other peptides derived from naturally occurring trimeric proteins may be employed in preparing trimeric TR4. In specific embodiments, antibodies of the invention bind TR4-fusion protein monomers and/or TR4 fusion protein trimers.

Antibodies of the invention that bind TR4 polypeptides may bind them in as isolated polypeptides, in their naturally occurring state and/or their native conformation. By “isolated polypeptide” is intended a polypeptide removed from its native environment. Thus, a polypeptide produced and/or contained within a recombinant host cell is considered isolated for purposes of the present invention. Also intended as an “isolated polypeptide” are polypeptides that have been purified, partially or substantially, from a recombinant host cell. Thus, antibodies of the present invention may bind recombinantly produced TR4 polypeptides.

Antibodies of the present invention may also bind TR4 expressed on the surface of a cell, wherein said TR4 polypeptide is encoded by a polynucleotide encoding amino acids 1 to 468 of SEQ ID NO:2 operably associated with a regulatory sequence that controls expression of said polynucleotide.

Antibodies of the present invention may bind TR4 polypeptide fragments comprising or alternatively, consisting of, an amino acid sequence contained in SEQ ID NO:1, encoded by the cDNA contained in ATCC deposit Number 97853, or encoded by nucleic acids which hybridize (e.g., under stringent hybridization conditions) to the nucleotide sequence contained in ATCC deposit Number 97853, or the complementary strand thereto. Protein fragments may be “free-standing,” or comprised within a larger polypeptide of which the fragment forms a part or region, most preferably as a single continuous region. Antibodies of the present invention may bind polypeptide fragments, including, for example, fragments that comprise or alternatively, consist of from about amino acid residues: 1 to 23, 24 to 43, 44 to 63, 64 to 83, 84 to 103, 104 to 123, 124 to 143, 144 to 163, 164 to 183, 184 to 203, 204 to 223, 224 to 238, 239 to 264, 265 to 284, 285 to 304, 305 to 324, 325 to 345, 346 to 366, 367 to 387, 388 to 418, 419 to 439, and/or 440 to 468 of SEQ ID NO:1. In this context “about” includes the particularly recited value, larger or smaller by several (5, 4, 3, 2, or 1) amino acids, at either extreme or at both extremes. Moreover, polypeptide fragments bound by the antibodies of the invention can be at least about 10, 20, 30, 40, 50, 60, 70, 80, 90, 100, 110, 120, 130, 140, 150, 175 or 200 amino acids in length. In this context “about” includes the particularly recited value, larger or smaller by several (5, 4, 3, 2, or 1) amino acids, at either extreme or at both extremes.

Preferably, antibodies of the present invention bind polypeptide fragments selected from the group: a polypeptide comprising or alternatively, consisting of, the TR4 receptor extracellular domain (predicted to constitute amino acid residues from about 24 to about 238 in SEQ ID NO:1); a polypeptide comprising or alternatively, consisting of, both TR4 cysteine rich domains (both of which may be found in the protein fragment consisting of amino acid residues from about 131 to about 229 in SEQ ID NO:1); a polypeptide comprising or alternatively, consisting of, the TR4 cysteine rich domain consisting of amino acid residues from about 131 to about 183 in SEQ ID NO:1); a polypeptide comprising or alternatively, consisting of, the TR4 cysteine rich domain consisting of amino acid residues from about 184 to about 229 in SEQ ID NO:1); a polypeptide comprising or alternatively, consisting of, the TR4 receptor transmembrane domain (predicted to constitute amino acid residues from about 239 to about 264 in SEQ ID NO:1); a polypeptide comprising or alternatively, consisting of, fragment of the predicted mature TR4 polypeptide, wherein the fragment has a TR4 functional activity (e.g., antigenic activity or biological activity); a polypeptide comprising or alternatively, consisting of, the TR4 receptor intracellular domain (predicted to constitute amino acid residues from about 265 to about 468 in SEQ ID NO:1); a polypeptide comprising or alternatively, consisting of, the TR4 receptor extracellular and intracellular domains with all or part of the transmembrane domain deleted; a polypeptide comprising, or alternatively consisting of, the TR4 receptor death domain (predicted to constitute amino acid residues from about 379 to about 422 in SEQ ID NO:1); and a polypeptide comprising, or alternatively, consisting of, one, two, three, four or more, epitope bearing portions of the TR4 receptor protein. In additional embodiments, the polypeptide fragments of the invention comprise, or alternatively, consist of, any combination of 1, 2, 3, 4, 5, 6, 7, or all 8 of the above members. The amino acid residues constituting the TR4 receptor extracellular, transmembrane and intracellular domains have been predicted by computer analysis. Thus, as one of ordinary skill would appreciate, the amino acid residues constituting these domains may vary slightly (e.g., by about 1 to about 15 amino acid residues) depending on the criteria used to define each domain. Polynucleotides encoding these polypeptides are also encompassed by the invention.

It is believed that one or both of the extracellular cysteine rich motifs of TR4 is important for interactions between TR4 and its ligands (e.g., TRAIL). Accordingly, in highly preferred embodiments, antibodies of the present invention bind TR4 polypeptide fragments comprising, or alternatively consisting of amino acid residues 131 to 183, and/or 184 to 229 of SEQ ID NO:1. In another highly preferred embodiment, antibodies of the present invention bind TR4 polypeptides comprising, or alternatively consisting of both of the extracellular cysteine rich motifs (amino acid residues 131 to 229 of SEQ ID NO:1.) In another preferred embodiment, antibodies of the present invention bind TR4 polypeptides comprising, or alternatively consisting the extracellular soluble domain of TR4 (amino acid residues 24-238 of SEQ ID NO:1.) In highly preferred embodiments, the antibodies of the invention that bind all or a portion of the extracellular soluble domain of TR4 (e.g., one or both cysteine rich domains) prevent TRAIL ligand from binding to TR4. In other highly preferred embodiments, the antibodies of the invention that bind all or a portion of the extracellular soluble domain of TR4 (e.g., one or both cysteine rich domains) agonize the TR4 receptor. In other highly preferred embodiments, the antibodies of the invention that bind all or a portion of the extracellular soluble domain of TR4 (e.g., one or both cysteine rich domains) induce cell death of the cell expressing the TR4 receptor.

Antibodies of the invention may also bind fragments comprising, or alternatively, consisting of structural or functional attributes of TR4. Such fragments include amino acid residues that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, surface forming regions, and high antigenic index regions (i.e., containing four or more contiguous amino acids having an antigenic index of greater than or equal to 1.5, as identified using the default parameters of the Jameson-Wolf program) of complete (i.e., full-length) TR4.

Certain preferred regions are those set out in Table 2 and include, but are not limited to, regions of the aforementioned types identified by analysis of the amino acid sequence depicted in (SEQ ID NO:1), such preferred regions include; Garnier-Robson predicted alpha-regions, beta-regions, turn-regions, and coil-regions; Chou-Fasman predicted alpha-regions, beta-regions, and turn-regions; Kyte-Doolittle predicted hydrophilic regions; Eisenberg alpha and beta amphipathic regions; Emini surface-forming regions; and Jameson-Wolf high antigenic index regions, as predicted using the default parameters of these computer programs.

The data representing the structural or functional attributes of TR4 set forth in Table 2, as described above, was generated using the various modules and algorithms of the DNA*STAR set on default parameters. Column I represents the results of a Garnier-Robson analysis of alpha helical regions; Column II represents the results of a Chou-Fasman analysis of alpha helical regions; Column III represents the results of a Garnier Robson analysis of beta sheet regions; Column IV represents the results of a Chou-Fasman analysis of beta sheet regions; Column V represents the results of a Garnier Robson analysis of turn regions; Column VI represents the results of a Chou-Fasman analysis of turn regions; Column VII represents the results of a Garnier Robson analysis of coil regions; Column VIII represents a Kyte-Doolittle hydrophilicity plot; Column; Column IX represents the results of an Eisenberg analysis of alpha amphipathic regions; Column X represents the results of an Eisenberg analysis of beta amphipathic regions; Column XI represents the results of a Karplus-Schultz analysis of flexible regions; Column XI represents the Jameson-Wolf antigenic index score; and Column XIII represents the Emini surface probability plot.

In a preferred embodiment, the data presented in columns VIII, XII, and XIII of Table 2 can be used to determine regions of TR4 which exhibit a high degree of potential for antigenicity. Regions of high antigenicity are determined from the data presented in columns VIII, XII, and/or XIII by choosing values which represent regions of the polypeptide which are likely to be exposed on the surface of the polypeptide in an environment in which antigen recognition may occur in the process of initiation of an immune response.

The above-mentioned preferred regions set out in Table 2 include, but are not limited to, regions of the aforementioned types identified by analysis of the amino acid sequence set out in SEQ ID NO:1. As set out in Table 2, such preferred regions include Garnier-Robson alpha-regions, beta-regions, turn-regions, and coil-regions, Chou-Fasman alpha-regions, beta-regions, and turn-regions, Kyte-Doolittle hydrophilic regions, Eisenberg alpha- and beta-amphipathic regions, Karplus-Schulz flexible regions, Jameson-Wolf regions of high antigenic index and Emini surface-forming regions. Among preferred polypeptide fragments bound by one or more antibodies of the invention are those that comprise regions of TR4 that combine several structural features, such as several (e.g., 1, 2, 3, or 4) of the same or different region features set out above and in Table 2.

TABLE 2

| Res | Position | I | II | III | IV | V | VI | VII | VIII | IX | X | XI | XII | XIII |
|---|---|---|---|---|---|---|---|---|---|---|---|---|---|---|
| Met | 1 | . | . | B | . | . | . | . | 0.12 | . | . | . | −0.10 | 0.90 |
| Ala | 2 | . | . | . | . | . | . | C | −0.08 | * | * | . | 0.25 | 1.08 |
| Pro | 3 | . | . | . | . | . | . | C | 0.42 | * | * | . | 0.10 | 0.86 |
| Pro | 4 | . | . | . | . | . | T | C | −0.04 | * | * | . | 1.05 | 1.69 |
| Pro | 5 | A | . | . | . | . | T | . | 0.31 | . | * | F | 1.00 | 1.24 |
| Ala | 6 | A | . | . | . | . | T | . | 0.10 | . | * | F | 1.00 | 1.10 |
| Arg | 7 | A | . | . | . | . | T | . | 0.34 | . | * | . | 0.10 | 0.58 |
| Val | 8 | . | . | B | B | . | . | . | −0.03 | . | * | . | −0.30 | 0.37 |
| His | 9 | . | . | B | B | . | . | . | −0.52 | . | * | . | −0.30 | 0.37 |
| Leu | 10 | . | . | B | B | . | . | . | −1.12 | . | * | . | −0.60 | 0.17 |
| Gly | 11 | . | . | B | B | . | . | . | −1.12 | . | * | . | −0.60 | 0.18 |
| Ala | 12 | . | . | B | B | . | . | . | −2.09 | . | * | . | −0.60 | 0.14 |
| Phe | 13 | . | . | B | B | . | . | . | −1.54 | . | * | . | −0.60 | 0.12 |
| Leu | 14 | . | . | B | B | . | . | . | −1.72 | . | . | . | −0.60 | 0.18 |
| Ala | 15 | . | . | B | B | . | . | . | −0.91 | . | . | . | −0.60 | 0.27 |
| Val | 16 | . | . | B | B | . | . | . | −0.78 | . | . | . | −0.60 | 0.51 |
| Thr | 17 | . | . | B | B | . | . | . | −0.53 | . | . | F | −0.45 | 0.95 |
| Pro | 18 | . | . | . | B | . | . | C | −0.13 | . | . | F | 0.05 | 0.93 |
| Asn | 19 | . | . | . | . | . | T | C | 0.09 | . | . | F | 0.60 | 1.69 |
| Pro | 20 | . | . | . | . | . | T | C | 0.09 | . | . | F | 0.60 | 1.18 |
| Gly | 21 | . | . | . | . | T | T | . | 0.64 | . | . | F | 0.65 | 0.77 |
| Ser | 22 | . | . | . | . | . | T | C | 0.61 | . | . | F | 0.45 | 0.64 |
| Ala | 23 | . | . | . | . | . | . | C | 0.51 | . | . | F | 0.25 | 0.41 |
| Ala | 24 | . | . | . | . | . | T | C | 0.51 | . | . | F | 0.45 | 0.60 |
| Ser | 25 | . | . | B | . | . | T | . | 0.13 | . | . | F | 0.85 | 0.78 |
| Gly | 26 | A | . | . | . | . | T | . | −0.11 | . | . | F | 0.85 | 0.78 |
| Thr | 27 | A | . | . | . | . | T | . | −0.40 | . | . | F | 0.85 | 0.78 |
| Glu | 28 | A | A | . | . | . | . | . | −0.40 | . | . | F | 0.45 | 0.58 |
| Ala | 29 | A | A | . | . | . | . | . | −0.12 | . | . | . | 0.30 | 0.60 |
| Ala | 30 | A | A | . | . | . | . | . | −0.03 | . | . | . | 0.30 | 0.60 |
| Ala | 31 | A | A | . | . | . | . | . | 0.01 | . | . | . | 0.30 | 0.53 |
| Ala | 32 | A | A | . | . | . | . | . | 0.37 | . | . | . | −0.30 | 0.71 |
| Thr | 33 | A | . | . | . | . | T | . | −0.49 | * | . | F | 1.00 | 1.40 |
| Pro | 34 | A | . | . | . | . | T | . | −0.19 | . | . | F | 1.00 | 1.03 |
| Ser | 35 | . | . | B | . | . | T | . | 0.06 | . | . | F | 0.40 | 1.07 |
| Lys | 36 | . | . | B | . | . | T | . | 0.34 | . | . | F | 0.25 | 0.73 |
| Val | 37 | . | . | B | B | . | . | . | 0.63 | . | . | F | −0.15 | 0.64 |
| Trp | 38 | . | . | B | B | . | . | . | 0.36 | . | . | F | −0.15 | 0.64 |
| Gly | 39 | . | . | B | B | . | . | . | 0.22 | * | * | F | −0.15 | 0.32 |
| Ser | 40 | . | . | . | . | . | . | C | 0.63 | * | * | F | −0.05 | 0.43 |
| Ser | 41 | . | . | . | . | . | T | C | −0.30 | * | * | F | 0.45 | 0.80 |
| Ala | 42 | . | . | . | . | . | T | C | 0.56 | * | * | F | 1.05 | 0.57 |
| Gly | 43 | . | . | . | . | . | T | C | 0.63 | * | * | F | 1.35 | 0.73 |
| Arg | 44 | . | . | B | . | . | T | . | 1.09 | * | * | F | 1.49 | 0.84 |
| Ile | 45 | . | . | B | . | . | . | . | 1.04 | * | * | F | 1.78 | 1.63 |
| Glu | 46 | . | . | B | . | . | . | . | 1.00 | * | * | F | 2.12 | 1.63 |
| Pro | 47 | . | . | B | . | . | T | . | 1.24 | * | * | F | 2.51 | 0.83 |
| Arg | 48 | . | . | . | . | T | T | . | 1.70 | * | * | F | 3.40 | 1.17 |
| Gly | 49 | . | . | . | . | T | T | . | 1.24 | * | * | F | 3.06 | 1.32 |
| Gly | 50 | . | . | . | . | T | T | . | 1.54 | * | * | F | 2.57 | 0.84 |
| Gly | 51 | . | . | . | . | . | T | C | 0.73 | * | * | F | 2.03 | 0.44 |
| Arg | 52 | . | . | . | . | . | T | C | 0.73 | * | * | F | 1.39 | 0.36 |
| Gly | 53 | . | . | B | . | . | T | . | 0.31 | * | * | F | 0.85 | 0.57 |
| Ala | 54 | . | . | B | . | . | T | . | 0.36 | . | * | F | 0.85 | 0.83 |
| Leu | 55 | . | . | B | . | . | . | . | 0.10 | . | * | F | 0.65 | 0.57 |
| Pro | 56 | . | . | B | . | . | . | . | 0.10 | . | * | F | −0.25 | 0.57 |
| Thr | 57 | . | . | B | . | . | . | . | −0.01 | . | * | F | −0.25 | 0.55 |
| Ser | 58 | . | . | B | . | . | T | . | 0.30 | . | . | F | 0.10 | 1.16 |
| Met | 59 | . | . | B | . | . | T | . | 0.54 | . | . | F | 0.40 | 1.02 |
| Gly | 60 | . | . | B | . | . | T | . | 1.14 | . | . | F | 0.25 | 0.70 |
| Gln | 61 | . | . | . | . | T | T | . | 1.06 | . | . | F | 0.65 | 0.81 |
| His | 62 | . | . | . | . | . | . | C | 0.78 | . | * | F | 0.40 | 1.10 |
| Gly | 63 | . | . | . | . | . | T | C | 1.19 | . | * | F | 0.60 | 1.12 |
| Pro | 64 | . | . | . | . | . | T | C | 1.20 | . | * | F | 1.20 | 1.27 |
| Ser | 65 | . | . | . | . | . | T | C | 1.66 | . | * | F | 1.05 | 0.94 |
| Ala | 66 | . | . | B | . | . | T | . | 1.07 | . | * | F | 1.30 | 1.86 |
| Arg | 67 | . | . | B | . | . | . | . | 0.76 | * | * | . | 1.29 | 1.22 |
| Ala | 68 | . | . | B | . | . | . | . | 1.21 | * | * | . | 1.48 | 0.90 |
| Arg | 69 | . | . | B | . | . | T | . | 0.83 | . | * | . | 2.17 | 1.74 |
| Ala | 70 | . | . | B | . | . | T | . | 0.92 | . | * | F | 2.51 | 0.90 |
| Gly | 71 | . | . | . | . | T | T | . | 1.17 | . | * | F | 3.40 | 1.37 |
| Arg | 72 | . | . | . | . | . | T | C | 0.84 | . | * | F | 2.71 | 0.69 |
| Ala | 73 | . | . | . | . | . | T | C | 1.54 | * | . | F | 2.48 | 1.06 |
| Pro | 74 | . | . | . | . | . | T | C | 1.22 | * | . | F | 2.70 | 2.10 |
| Gly | 75 | . | . | . | . | . | T | C | 1.22 | * | . | F | 2.62 | 1.66 |
| Pro | 76 | . | . | . | . | . | T | C | 1.68 | * | * | F | 2.24 | 1.66 |
| Arg | 77 | . | . | . | . | . | . | C | 1.57 | * | . | F | 2.60 | 2.10 |
| Pro | 78 | . | A | B | . | . | . | . | 1.57 | * | . | F | 1.94 | 3.68 |
| Ala | 79 | . | A | B | . | . | . | . | 1.48 | * | . | F | 1.68 | 2.40 |
| Arg | 80 | . | A | B | . | . | . | . | 1.61 | * | * | F | 1.42 | 1.64 |
| Glu | 81 | . | A | B | . | . | . | . | 1.93 | * | * | F | 1.16 | 1.64 |
| Ala | 82 | A | A | . | . | . | . | . | 1.01 | * | * | F | 0.90 | 3.19 |
| Ser | 83 | A | . | . | . | . | T | . | 1.33 | * | * | F | 1.30 | 1.34 |
| Pro | 84 | A | . | . | . | . | T | . | 1.07 | * | * | F | 1.30 | 1.52 |
| Arg | 85 | A | . | . | . | . | T | . | 0.92 | * | * | F | 1.00 | 1.12 |
| Leu | 86 | A | . | . | . | . | T | . | 0.97 | . | * | . | 0.85 | 1.13 |
| Arg | 87 | A | . | . | B | . | . | . | 1.24 | . | * | . | 0.75 | 1.46 |
| Val | 88 | A | . | . | B | . | . | . | 0.84 | * | * | . | 0.75 | 1.08 |
| His | 89 | A | . | . | B | . | . | . | 1.10 | . | * | . | −0.15 | 1.13 |
| Lys | 90 | A | . | . | B | . | . | . | 0.29 | * | * | F | 0.90 | 1.16 |
| Thr | 91 | . | . | B | B | . | . | . | 0.24 | * | * | F | 0.00 | 1.35 |
| Phe | 92 | . | . | B | B | . | . | . | −0.72 | * | * | . | −0.30 | 0.74 |
| Lys | 93 | . | . | B | B | . | . | . | −0.72 | * | * | . | −0.30 | 0.27 |
| Phe | 94 | . | . | B | B | . | . | . | −1.03 | * | . | . | −0.60 | 0.14 |
| Val | 95 | . | . | B | B | . | . | . | −1.93 | * | . | . | −0.60 | 0.16 |
| Val | 96 | . | . | B | B | . | . | . | −2.43 | . | * | . | −0.60 | 0.06 |
| Val | 97 | . | . | B | B | . | . | . | −2.54 | . | * | . | −0.60 | 0.06 |
| Gly | 98 | . | . | B | B | . | . | . | −2.59 | . | * | . | −0.60 | 0.06 |
| Val | 99 | . | . | B | B | . | . | . | −2.74 | . | . | . | −0.60 | 0.15 |
| Leu | 100 | . | . | B | B | . | . | . | −2.74 | * | . | . | −0.60 | 0.15 |
| Leu | 101 | . | . | B | B | . | . | . | −2.10 | * | . | . | −0.60 | 0.11 |
| Gln | 102 | . | . | B | B | . | . | . | −1.54 | * | . | . | −0.60 | 0.23 |
| Val | 103 | . | . | B | B | . | . | . | −1.50 | . | . | . | −0.60 | 0.37 |
| Val | 104 | . | . | B | . | . | T | . | −1.23 | . | . | . | −0.20 | 0.61 |
| Pro | 105 | . | . | B | . | . | T | . | −1.01 | * | . | F | 0.25 | 0.35 |
| Ser | 106 | A | . | . | . | . | T | . | −0.51 | * | . | F | −0.05 | 0.48 |
| Ser | 107 | A | . | . | . | . | T | . | −1.40 | * | * | F | 0.25 | 0.94 |
| Ala | 108 | A | . | . | . | . | . | . | −0.50 | . | * | F | 0.05 | 0.43 |
| Ala | 109 | A | . | . | . | . | . | . | −0.46 | . | * | . | 0.50 | 0.63 |
| Thr | 110 | A | . | . | . | . | . | . | −0.28 | . | * | . | −0.10 | 0.39 |
| Ile | 111 | A | . | . | . | . | . | . | 0.02 | . | * | . | −0.10 | 0.53 |
| Lys | 112 | . | . | B | . | . | . | . | 0.32 | . | * | . | 0.50 | 0.87 |
| Leu | 113 | . | . | B | . | . | . | . | 0.61 | . | * | F | 1.05 | 1.04 |
| His | 114 | . | . | B | . | . | . | . | 0.31 | . | * | F | 1.30 | 1.99 |
| Asp | 115 | . | . | . | . | . | T | C | 0.28 | * | * | F | 1.80 | 0.70 |
| Gln | 116 | . | . | . | . | T | T | . | 0.86 | . | * | F | 1.65 | 0.84 |
| Ser | 117 | . | . | . | . | T | T | . | 0.81 | . | . | F | 2.50 | 0.89 |
| Ile | 118 | . | . | . | . | T | T | . | 1.62 | . | . | F | 2.25 | 0.92 |
| Gly | 119 | . | . | . | . | . | . | C | 1.37 | . | . | F | 1.00 | 0.92 |
| Thr | 120 | . | . | . | . | . | . | C | 1.37 | . | . | F | 0.45 | 0.72 |
| Gln | 121 | . | . | B | . | . | . | C | 1.33 | . | . | F | 0.65 | 1.79 |
| Gln | 122 | . | . | B | . | . | . | . | 1.33 | . | . | F | 0.20 | 2.46 |
| Trp | 123 | . | . | B | . | . | . | . | 2.01 | . | . | . | 0.05 | 2.28 |
| Glu | 124 | . | . | . | . | . | . | C | 1.54 | . | . | . | 0.25 | 2.04 |
| His | 125 | . | . | . | . | . | . | C | 1.51 | . | . | . | 0.10 | 0.97 |
| Ser | 126 | . | . | . | . | . | T | C | 1.51 | . | . | F | 0.45 | 0.91 |
| Pro | 127 | . | . | . | . | T | T | . | 0.70 | . | . | F | 1.55 | 0.91 |
| Leu | 128 | . | . | . | . | T | T | . | 0.32 | . | . | F | 0.65 | 0.55 |
| Gly | 129 | . | . | . | . | T | T | . | 0.11 | . | . | F | 0.65 | 0.22 |
| Glu | 130 | . | . | . | . | T | . | . | −0.07 | . | . | F | 0.45 | 0.22 |
| Leu | 131 | . | . | B | . | . | . | . | −0.11 | * | . | . | 0.18 | 0.42 |
| Cys | 132 | . | . | B | . | . | . | . | −0.20 | * | . | F | 1.21 | 0.42 |
| Pro | 133 | . | . | B | . | . | T | . | 0.58 | * | * | F | 1.69 | 0.32 |
| Pro | 134 | . | . | . | . | T | T | . | 1.03 | . | * | F | 1.47 | 0.53 |
| Gly | 135 | . | . | . | . | T | T | . | 0.73 | . | * | F | 2.80 | 1.94 |
| Ser | 136 | . | . | . | . | . | T | C | 1.54 | * | . | F | 2.32 | 1.68 |
| His | 137 | . | . | . | . | . | . | C | 2.32 | * | . | F | 2.48 | 1.88 |
| Arg | 138 | . | . | B | . | . | . | . | 2.32 | * | . | F | 2.34 | 3.72 |
| Ser | 139 | . | . | B | . | . | . | . | 2.19 | * | . | F | 2.40 | 4.29 |
| Glu | 140 | . | . | . | . | T | . | . | 1.94 | * | . | F | 2.86 | 3.12 |
| Arg | 141 | . | . | . | . | T | T | . | 1.58 | * | . | F | 3.40 | 1.61 |
| Pro | 142 | . | . | . | . | T | T | . | 1.61 | . | * | F | 2.91 | 0.64 |
| Gly | 143 | . | . | . | . | T | T | . | 1.61 | . | * | F | 2.57 | 0.60 |
| Ala | 144 | . | . | . | . | T | T | . | 1.24 | . | * | . | 2.08 | 0.60 |
| Cys | 145 | . | . | . | . | T | . | . | 0.93 | . | * | . | 1.41 | 0.21 |
| Asn | 146 | . | . | B | . | . | . | . | 0.82 | . | * | . | 0.84 | 0.30 |
| Arg | 147 | . | . | B | . | . | . | . | 0.69 | * | . | . | 1.01 | 0.52 |
| Cys | 148 | . | . | B | . | . | T | . | 0.18 | * | . | F | 1.83 | 0.96 |
| Thr | 149 | . | . | B | . | . | T | . | 0.42 | * | . | F | 1.70 | 0.44 |
| Glu | 150 | . | . | B | . | . | T | . | 0.84 | * | . | F | 1.53 | 0.22 |
| Gly | 151 | . | . | B | . | . | T | . | 0.53 | * | . | F | 0.76 | 0.65 |
| Val | 152 | . | . | B | B | . | . | . | 0.42 | . | * | F | 0.19 | 0.65 |
| Gly | 153 | . | . | B | B | . | . | . | 0.50 | . | . | . | −0.13 | 0.61 |
| Tyr | 154 | . | . | B | B | . | . | . | 0.51 | . | . | . | −0.60 | 0.62 |
| Thr | 155 | . | . | B | B | . | . | . | 0.51 | . | . | F | −0.30 | 1.12 |
| Asn | 156 | . | . | . | B | . | . | C | 0.86 | . | . | F | 0.20 | 1.81 |
| Ala | 157 | . | . | . | . | T | T | . | 0.90 | . | . | F | 0.80 | 1.86 |
| Ser | 158 | . | . | . | . | T | T | . | 0.54 | . | . | F | 0.80 | 1.06 |
| Asn | 159 | . | . | . | . | T | T | . | 0.20 | . | . | F | 0.35 | 0.57 |
| Asn | 160 | . | . | . | . | T | T | . | −0.16 | * | . | F | 0.35 | 0.57 |
| Leu | 161 | . | A | B | . | . | . | . | −0.97 | * | . | . | −0.60 | 0.23 |
| Phe | 162 | . | A | B | . | . | . | . | −0.59 | . | . | . | −0.60 | 0.12 |
| Ala | 163 | . | A | B | . | . | . | . | −0.96 | . | . | . | −0.60 | 0.11 |
| Cys | 164 | . | A | B | . | . | . | . | −1.27 | * | . | . | −0.60 | 0.07 |
| Leu | 165 | . | . | B | . | . | T | . | −1.86 | . | . | . | −0.20 | 0.12 |
| Pro | 166 | . | . | B | . | . | T | . | −1.71 | * | . | . | −0.20 | 0.12 |
| Cys | 167 | . | . | . | . | T | T | . | −0.97 | * | . | . | 0.20 | 0.12 |
| Thr | 168 | A | . | . | . | . | T | . | −0.68 | . | . | . | 0.10 | 0.30 |
| Ala | 169 | A | . | . | . | . | . | . | −0.01 | . | . | . | 0.50 | 0.26 |
| Cys | 170 | A | . | . | . | . | T | . | 0.80 | . | . | . | 0.70 | 0.80 |
| Lys | 171 | A | . | . | . | . | T | . | 1.01 | . | . | F | 1.15 | 0.96 |
| Ser | 172 | A | . | . | . | . | T | . | 1.68 | . | * | F | 1.30 | 1.65 |
| Asp | 173 | A | . | . | . | . | T | . | 2.10 | . | * | F | 1.30 | 5.33 |
| Glu | 174 | A | A | . | . | . | . | . | 2.39 | . | * | F | 0.90 | 5.22 |
| Glu | 175 | A | A | . | . | . | . | . | 2.84 | . | * | F | 1.24 | 5.22 |
| Glu | 176 | A | A | . | . | . | . | . | 2.13 | . | * | F | 1.58 | 4.83 |
| Arg | 177 | . | A | . | . | T | . | . | 2.12 | . | . | F | 2.32 | 1.50 |
| Ser | 178 | . | . | . | . | . | T | C | 1.81 | . | . | F | 2.86 | 1.25 |
| Pro | 179 | . | . | . | . | T | T | . | 1.50 | * | . | F | 3.40 | 1.04 |
| Cys | 180 | . | . | . | . | T | T | . | 1.61 | * | . | F | 2.61 | 0.77 |
| Thr | 181 | . | . | . | . | T | T | . | 1.61 | * | . | F | 2.67 | 1.12 |
| Thr | 182 | . | . | . | . | T | . | . | 1.19 | * | * | F | 2.38 | 1.16 |
| Thr | 183 | . | . | . | . | T | T | . | 0.90 | . | . | F | 2.49 | 3.13 |
| Arg | 184 | . | . | . | . | T | T | . | 0.44 | . | . | F | 2.40 | 2.19 |
| Asn | 185 | . | . | . | . | T | T | . | 1.11 | . | . | F | 2.50 | 0.81 |
| Thr | 186 | . | . | . | . | T | T | . | 0.76 | * | . | F | 2.25 | 0.98 |
| Ala | 187 | . | . | . | . | T | . | . | 1.11 | * | . | . | 1.65 | 0.27 |
| Cys | 188 | . | . | . | . | T | . | . | 1.21 | * | . | . | 1.40 | 0.33 |
| Gln | 189 | . | . | B | . | . | . | . | 0.76 | * | . | . | 0.75 | 0.36 |
| Cys | 190 | . | . | B | . | . | . | . | 0.44 | . | . | . | 0.50 | 0.35 |
| Lys | 191 | . | . | B | . | . | T | . | 0.06 | . | * | F | 0.85 | 0.94 |
| Pro | 192 | . | . | . | . | T | T | . | 0.76 | . | . | F | 0.65 | 0.47 |
| Gly | 193 | . | . | . | . | T | T | . | 1.42 | . | * | F | 1.74 | 1.72 |
| Thr | 194 | . | . | B | . | . | T | . | 1.42 | . | * | F | 1.68 | 1.38 |
| Phe | 195 | . | . | B | . | . | . | . | 2.09 | . | * | F | 1.82 | 1.49 |
| Arg | 196 | . | . | . | . | T | . | . | 1.74 | . | * | F | 2.56 | 2.42 |
| Asn | 197 | . | . | . | . | T | T | . | 1.37 | . | * | F | 3.40 | 2.25 |
| Asp | 198 | . | . | . | . | T | T | . | 1.71 | . | * | F | 3.06 | 2.63 |
| Asn | 199 | . | . | . | . | . | T | C | 1.42 | . | * | F | 2.52 | 2.32 |
| Ser | 200 | A | . | . | . | . | T | . | 1.46 | . | * | F | 1.98 | 1.43 |
| Ala | 201 | A | . | . | . | . | . | . | 1.46 | . | * | . | 1.14 | 0.46 |
| Glu | 202 | A | . | . | . | . | . | . | 1.50 | * | . | . | 0.80 | 0.56 |
| Met | 203 | A | . | . | . | . | . | . | 0.83 | * | . | . | 1.11 | 0.83 |
| Cys | 204 | A | . | . | . | . | T | . | 0.53 | * | . | . | 1.62 | 0.44 |
| Arg | 205 | . | . | . | . | T | T | . | 0.52 | * | . | . | 2.33 | 0.34 |
| Lys | 206 | . | . | . | . | T | T | . | 0.77 | * | . | F | 2.49 | 0.50 |
| Cys | 207 | . | . | . | . | T | T | . | 0.10 | * | . | F | 3.10 | 0.92 |
| Ser | 208 | . | . | . | . | T | . | . | 0.49 | * | * | F | 2.59 | 0.25 |
| Thr | 209 | . | . | . | . | T | . | . | 1.27 | * | * | F | 1.98 | 0.19 |
| Gly | 210 | . | . | . | . | T | . | . | 0.81 | * | . | F | 1.67 | 0.71 |
| Cys | 211 | . | . | B | . | . | T | . | 0.17 | * | * | F | 1.16 | 0.53 |
| Pro | 212 | . | . | . | . | T | T | . | −0.02 | * | * | F | 1.25 | 0.36 |
| Arg | 213 | . | . | . | . | T | T | . | 0.32 | * | * | F | 0.65 | 0.27 |
| Gly | 214 | . | . | B | . | . | T | . | −0.22 | * | * | . | 0.85 | 1.01 |
| Met | 215 | . | . | B | B | . | . | . | 0.17 | * | * | . | 0.30 | 0.48 |
| Val | 216 | . | . | B | B | . | . | . | 0.83 | * | * | . | 0.79 | 0.49 |
| Lys | 217 | . | . | B | B | . | . | . | 0.38 | * | * | . | 0.98 | 0.83 |
| Val | 218 | . | . | B | B | . | . | . | −0.04 | * | * | F | 1.32 | 0.45 |
| Lys | 219 | . | . | B | B | . | . | . | 0.09 | . | * | F | 1.51 | 0.88 |
| Asp | 220 | . | . | B | . | . | . | . | 0.40 | . | * | F | 1.90 | 0.68 |
| Cys | 221 | . | . | B | . | . | . | . | 0.96 | . | * | F | 0.81 | 0.96 |
| Thr | 222 | . | . | . | . | . | T | C | 0.91 | . | * | F | 1.62 | 0.65 |
| Pro | 223 | . | . | . | . | T | T | . | 0.88 | . | * | F | 1.63 | 0.65 |
| Trp | 224 | . | . | . | . | T | T | . | 0.83 | . | * | F | 0.54 | 0.84 |
| Ser | 225 | A | . | . | . | . | T | . | 0.17 | . | . | F | 1.00 | 1.01 |
| Asp | 226 | A | A | . | . | . | . | . | −0.02 | . | . | F | 0.45 | 0.35 |
| Ile | 227 | A | A | . | . | . | . | . | 0.26 | * | . | . | −0.30 | 0.25 |
| Glu | 228 | A | A | . | . | . | . | . | 0.51 | * | . | . | 0.30 | 0.25 |
| Cys | 229 | . | A | B | . | . | . | . | 0.80 | * | . | . | 0.60 | 0.30 |
| Val | 230 | A | A | . | . | . | . | . | 0.80 | * | * | . | 0.60 | 0.74 |
| His | 231 | A | A | . | . | . | . | . | 0.46 | * | * | . | 0.60 | 0.58 |
| Lys | 232 | A | A | . | . | . | . | . | 1.34 | * | . | F | 0.60 | 1.06 |
| Glu | 233 | . | A | . | . | T | . | . | 1.00 | * | . | F | 1.30 | 2.30 |
| Ser | 234 | . | . | . | . | T | T | . | 1.63 | * | . | F | 1.70 | 1.68 |
| Gly | 235 | . | . | . | . | T | T | . | 2.49 | * | . | F | 1.70 | 1.14 |
| Asn | 236 | . | . | . | . | T | T | . | 1.63 | * | . | F | 1.40 | 1.06 |
| Gly | 237 | . | . | . | . | . | T | C | 1.30 | * | . | F | 0.45 | 0.55 |
| His | 238 | . | . | . | B | . | . | C | 0.44 | . | . | . | −0.40 | 0.59 |
| Asn | 239 | . | . | . | B | . | . | C | −0.14 | . | . | . | −0.40 | 0.27 |
| Ile | 240 | . | . | B | B | . | . | . | −0.61 | . | . | . | −0.60 | 0.19 |
| Trp | 241 | . | . | B | B | . | . | . | −1.47 | . | . | . | −0.60 | 0.12 |
| Val | 242 | . | . | B | B | . | . | . | −1.98 | . | . | . | −0.60 | 0.05 |
| Ile | 243 | . | . | B | B | . | . | . | −2.26 | . | . | . | −0.60 | 0.06 |
| Leu | 244 | . | . | B | B | . | . | . | −3.07 | . | . | . | −0.60 | 0.08 |
| Val | 245 | . | . | B | B | . | . | . | −3.03 | . | . | . | −0.60 | 0.09 |
| Val | 246 | . | . | B | B | . | . | . | −3.60 | . | . | . | −0.60 | 0.09 |
| Thr | 247 | . | . | B | B | . | . | . | −2.96 | . | . | . | −0.60 | 0.08 |
| Leu | 248 | . | . | B | B | . | . | . | −2.88 | . | . | . | −0.60 | 0.17 |
| Val | 249 | . | . | B | B | . | . | . | −2.88 | . | * | . | −0.60 | 0.19 |
| Val | 250 | . | . | B | B | . | . | . | −2.83 | . | . | . | −0.60 | 0.11 |
| Pro | 251 | . | . | B | B | . | . | . | −2.83 | . | . | . | −0.60 | 0.11 |
| Leu | 252 | . | . | B | B | . | . | . | −3.11 | . | . | . | −0.60 | 0.11 |
| Leu | 253 | A | . | . | B | . | . | . | −3.16 | . | . | . | −0.60 | 0.15 |
| Leu | 254 | A | . | . | B | . | . | . | −3.11 | . | . | . | −0.60 | 0.07 |
| Val | 255 | A | . | . | B | . | . | . | −3.14 | . | . | . | −0.60 | 0.07 |
| Ala | 256 | A | . | . | B | . | . | . | −3.79 | . | . | . | −0.60 | 0.06 |
| Val | 257 | . | . | B | B | . | . | . | −3.64 | . | . | . | −0.60 | 0.05 |
| Leu | 258 | . | . | B | B | . | . | . | −3.50 | . | . | . | −0.60 | 0.04 |
| Ile | 259 | . | . | B | B | . | . | . | −3.36 | . | . | . | −0.60 | 0.02 |
| Val | 260 | . | . | B | B | . | . | . | −3.39 | . | . | . | −0.60 | 0.02 |
| Cys | 261 | . | . | B | B | . | . | . | −3.14 | . | . | . | −0.60 | 0.01 |
| Cys | 262 | . | . | B | B | . | . | . | −2.59 | . | . | . | −0.60 | 0.02 |
| Cys | 263 | . | . | B | B | . | . | . | −2.12 | . | . | . | −0.60 | 0.03 |
| Ile | 264 | . | . | B | B | . | . | . | −1.90 | . | . | . | −0.60 | 0.06 |
| Gly | 265 | . | . | . | . | T | T | . | −1.39 | . | . | F | 0.35 | 0.06 |
| Ser | 266 | . | . | . | . | T | T | . | −1.07 | . | . | F | 0.35 | 0.11 |
| Gly | 267 | . | . | . | . | T | T | . | −0.40 | . | . | F | 0.65 | 0.16 |
| Cys | 268 | . | . | . | . | T | T | . | 0.06 | . | . | F | 1.25 | 0.27 |
| Gly | 269 | . | . | . | . | T | . | . | 0.99 | . | * | F | 1.39 | 0.31 |
| Gly | 270 | . | . | . | . | T | . | . | 0.67 | . | . | F | 2.03 | 0.62 |
| Asp | 271 | . | . | . | . | . | T | C | 0.37 | . | . | F | 2.37 | 0.62 |
| Pro | 272 | . | . | . | . | T | T | . | 0.71 | * | * | F | 2.91 | 0.62 |
| Lys | 273 | . | . | . | . | T | T | . | 1.49 | * | * | F | 3.40 | 1.05 |
| Cys | 274 | . | . | B | . | . | T | . | 0.98 | * | * | . | 2.51 | 1.23 |
| Met | 275 | . | . | B | B | . | . | . | 0.66 | * | * | . | 1.62 | 0.59 |
| Asp | 276 | . | . | B | B | . | . | . | −0.04 | * | * | . | 1.28 | 0.16 |
| Arg | 277 | . | . | B | B | . | . | . | −0.12 | . | * | . | 0.04 | 0.26 |
| Val | 278 | . | . | B | B | . | . | . | −0.06 | . | * | . | −0.60 | 0.27 |
| Cys | 279 | . | . | B | B | . | . | . | −0.20 | . | . | . | 0.30 | 0.32 |
| Phe | 280 | . | . | B | B | . | . | . | 0.06 | . | * | . | −0.60 | 0.13 |
| Trp | 281 | . | . | B | B | . | . | . | −0.76 | . | . | . | −0.60 | 0.18 |
| Arg | 282 | . | . | B | B | . | . | . | −1.68 | . | . | . | −0.60 | 0.28 |
| Leu | 283 | . | . | B | B | . | . | . | −0.71 | . | . | . | −0.60 | 0.26 |
| Gly | 284 | . | . | . | B | T | . | . | −0.39 | . | * | . | −0.20 | 0.49 |
| Leu | 285 | . | . | . | B | . | . | C | 0.10 | . | * | . | 0.50 | 0.25 |
| Leu | 286 | . | . | . | B | . | . | C | 0.04 | . | * | . | 0.20 | 0.46 |
| Arg | 287 | . | . | . | B | . | . | C | −0.66 | . | . | F | 0.65 | 0.46 |
| Gly | 288 | . | . | . | . | . | T | C | 0.16 | . | . | F | 1.35 | 0.57 |
| Pro | 289 | . | . | . | . | . | T | C | 0.50 | . | * | F | 2.70 | 1.19 |
| Gly | 290 | . | . | . | . | . | T | C | 1.31 | * | * | F | 3.00 | 1.01 |
| Ala | 291 | A | . | . | . | . | T | . | 1.53 | . | * | F | 2.50 | 1.65 |
| Glu | 292 | A | . | . | . | . | . | . | 1.39 | . | . | F | 2.00 | 1.08 |
| Asp | 293 | A | . | . | . | . | . | . | 1.73 | . | . | F | 1.70 | 1.48 |
| Asn | 294 | A | . | . | . | . | T | . | 1.94 | . | * | . | 1.45 | 2.36 |
| Ala | 295 | A | . | . | . | . | T | . | 1.40 | . | . | . | 1.15 | 2.36 |
| His | 296 | A | . | . | . | . | T | . | 1.18 | * | . | . | 1.00 | 0.99 |
| Asn | 297 | A | . | . | . | . | T | . | 0.88 | . | . | . | 0.10 | 0.51 |
| Glu | 298 | A | . | . | . | . | . | . | 0.88 | * | . | . | −0.10 | 0.67 |
| Ile | 299 | A | . | . | . | . | . | . | 0.29 | * | * | . | −0.10 | 0.80 |
| Leu | 300 | A | . | . | . | . | . | . | 0.88 | * | * | . | −0.10 | 0.50 |
| Ser | 301 | A | . | . | . | . | . | . | 0.61 | * | . | F | 0.65 | 0.48 |
| Asn | 302 | A | . | . | . | . | T | . | −0.20 | * | . | F | 0.25 | 0.92 |
| Ala | 303 | A | . | . | . | . | T | . | −0.50 | * | . | F | 0.25 | 0.92 |
| Asp | 304 | A | . | . | . | . | T | . | 0.08 | * | . | F | 0.85 | 0.92 |
| Ser | 305 | . | . | . | . | . | T | C | 0.19 | * | . | F | 1.05 | 0.83 |
| Leu | 306 | . | . | . | B | . | . | C | −0.37 | * | . | F | 0.05 | 0.71 |
| Ser | 307 | . | . | B | B | . | . | . | −0.67 | * | . | F | −0.15 | 0.31 |
| Thr | 308 | . | . | B | B | . | . | . | −0.08 | * | . | . | −0.60 | 0.31 |
| Phe | 309 | . | . | B | B | . | . | . | −0.08 | * | . | . | −0.30 | 0.66 |
| Val | 310 | A | . | . | B | . | . | . | 0.22 | . | . | F | −0.15 | 0.85 |
| Ser | 311 | A | A | . | . | . | . | . | 0.43 | . | . | F | 0.00 | 1.03 |
| Glu | 312 | A | A | . | . | . | . | . | 0.73 | . | . | F | 0.00 | 1.17 |
| Gln | 313 | A | A | . | . | . | . | . | 0.74 | . | . | F | 0.90 | 2.73 |
| Gln | 314 | A | A | . | . | . | . | . | 1.44 | . | . | F | 0.90 | 2.73 |
| Met | 315 | A | A | . | . | . | . | . | 2.30 | . | . | F | 0.90 | 2.73 |
| Glu | 316 | A | A | . | . | . | . | . | 2.39 | . | . | F | 0.90 | 2.73 |
| Ser | 317 | A | A | . | . | . | . | . | 1.80 | . | * | F | 0.90 | 2.44 |
| Gln | 318 | A | A | . | . | . | . | . | 1.80 | . | * | F | 0.90 | 2.49 |
| Glu | 319 | A | A | . | . | . | . | . | 0.99 | . | * | F | 0.90 | 2.40 |
| Pro | 320 | A | A | . | . | . | . | . | 1.28 | . | * | F | 0.90 | 1.48 |
| Ala | 321 | A | A | . | . | . | . | . | 0.93 | . | . | F | 0.60 | 1.23 |
| Asp | 322 | A | A | . | B | . | . | . | 0.38 | . | . | F | 0.45 | 0.70 |
| Leu | 323 | A | A | . | B | . | . | . | 0.07 | . | . | F | −0.15 | 0.34 |
| Thr | 324 | . | A | B | B | . | . | . | −0.79 | . | . | F | −0.15 | 0.48 |
| Gly | 325 | . | A | B | B | . | . | . | −0.58 | . | . | . | −0.30 | 0.21 |
| Val | 326 | . | . | B | B | . | . | . | −0.29 | . | . | . | −0.60 | 0.45 |
| Thr | 327 | . | . | B | B | . | . | . | −0.50 | . | . | . | −0.60 | 0.42 |
| Val | 328 | . | . | B | B | . | . | . | −0.03 | . | * | F | −0.17 | 0.65 |
| Gln | 329 | . | . | B | B | . | . | . | 0.28 | . | * | F | 0.11 | 0.87 |
| Ser | 330 | . | . | . | . | . | T | C | 0.03 | . | * | F | 2.04 | 1.05 |
| Pro | 331 | . | . | . | . | . | T | C | 0.89 | . | * | F | 2.32 | 1.42 |
| Gly | 332 | . | . | . | . | T | T | . | 0.53 | . | * | F | 2.80 | 1.42 |
| Glu | 333 | A | . | . | . | . | T | . | 0.58 | . | * | F | 1.97 | 0.57 |
| Ala | 334 | . | . | B | . | . | . | . | −0.23 | . | * | . | 0.74 | 0.30 |
| Gln | 335 | . | . | B | . | . | . | . | −0.28 | . | . | . | 0.46 | 0.25 |
| Cys | 336 | . | . | B | . | . | . | . | −0.28 | . | . | . | 0.18 | 0.14 |
| Leu | 337 | . | . | B | . | . | . | . | −0.52 | . | * | . | −0.40 | 0.22 |
| Leu | 338 | . | . | B | . | . | . | . | −0.52 | . | * | . | −0.40 | 0.13 |
| Gly | 339 | . | A | . | . | . | . | C | −0.52 | . | * | F | 0.05 | 0.42 |
| Pro | 340 | A | A | . | . | . | . | . | −0.52 | . | * | F | −0.15 | 0.51 |
| Ala | 341 | A | A | . | . | . | . | . | −0.20 | . | * | F | 0.60 | 1.07 |
| Glu | 342 | A | A | . | . | . | . | . | 0.31 | . | * | F | 0.90 | 1.07 |
| Ala | 343 | A | A | . | . | . | . | . | 1.12 | * | * | F | 0.75 | 0.93 |
| Glu | 344 | A | A | . | . | . | . | . | 1.58 | . | * | F | 0.90 | 1.60 |
| Gly | 345 | A | A | . | . | . | . | . | 1.90 | . | * | F | 0.90 | 1.80 |
| Ser | 346 | A | . | . | . | . | T | . | 2.60 | . | * | F | 1.30 | 3.50 |
| Gln | 347 | A | . | . | . | . | T | . | 1.79 | . | * | F | 1.30 | 3.96 |
| Arg | 348 | A | . | . | . | . | T | . | 1.57 | . | * | F | 1.30 | 3.30 |
| Arg | 349 | . | . | B | . | . | T | . | 0.71 | . | * | F | 1.30 | 2.03 |
| Arg | 350 | . | . | B | B | . | . | . | 0.84 | . | * | F | 0.75 | 0.87 |
| Leu | 351 | . | . | B | B | . | . | . | 0.56 | . | * | . | 0.60 | 0.69 |
| Leu | 352 | . | . | B | B | . | . | . | 0.56 | . | * | . | 0.30 | 0.35 |
| Val | 353 | . | . | B | B | . | . | . | 0.10 | * | * | . | −0.30 | 0.29 |
| Pro | 354 | . | . | B | . | . | T | . | −0.60 | * | . | . | −0.20 | 0.35 |
| Ala | 355 | . | . | . | . | T | T | . | −0.71 | . | * | . | 0.50 | 0.43 |
| Asn | 356 | . | . | . | . | . | T | C | −0.11 | . | . | F | 1.65 | 0.96 |
| Gly | 357 | . | . | . | . | . | T | C | 0.39 | . | . | F | 1.95 | 0.96 |
| Ala | 358 | . | . | . | . | . | . | C | 1.24 | . | . | F | 2.20 | 1.37 |
| Asp | 359 | . | . | . | . | . | T | C | 1.14 | . | . | F | 3.00 | 1.48 |
| Pro | 360 | A | . | . | . | . | T | . | 0.92 | * | . | F | 2.50 | 2.16 |
| Thr | 361 | A | . | . | . | . | T | . | 0.32 | . | . | F | 1.90 | 1.76 |
| Glu | 362 | A | . | . | . | . | T | . | −0.14 | . | . | F | 1.60 | 1.04 |
| Thr | 363 | A | . | . | B | . | . | . | −0.26 | . | . | F | 0.15 | 0.56 |
| Leu | 364 | A | . | . | B | . | . | . | −0.96 | * | . | . | −0.60 | 0.33 |
| Met | 365 | A | . | . | B | . | . | . | −0.74 | * | . | . | −0.60 | 0.17 |
| Leu | 366 | A | . | . | B | . | . | . | −0.39 | * | . | . | −0.60 | 0.19 |
| Phe | 367 | A | . | . | B | . | . | . | −1.09 | * | . | . | −0.60 | 0.47 |
| Phe | 368 | A | . | . | B | . | . | . | −1.37 | * | . | . | −0.60 | 0.41 |
| Asp | 369 | A | . | . | B | . | . | . | −0.56 | * | . | . | −0.60 | 0.50 |
| Lys | 370 | A | A | . | . | . | . | . | −0.84 | * | . | . | −0.30 | 0.93 |
| Phe | 371 | A | A | . | B | . | . | . | −0.89 | * | . | . | −0.30 | 0.75 |
| Ala | 372 | A | A | . | B | . | . | . | −0.40 | * | . | . | −0.30 | 0.34 |
| Asn | 373 | . | A | B | B | . | . | . | −0.40 | * | . | . | −0.60 | 0.26 |
| Ile | 374 | . | A | B | B | . | . | . | −0.40 | * | . | . | −0.60 | 0.26 |
| Val | 375 | . | A | B | B | . | . | . | −0.74 | . | . | . | −0.60 | 0.43 |
| Pro | 376 | . | A | . | B | . | . | C | −0.33 | . | . | . | −0.10 | 0.36 |
| Phe | 377 | . | . | . | . | T | T | . | 0.26 | . | . | . | 0.20 | 0.54 |
| Asp | 378 | . | . | . | . | T | T | . | 0.26 | . | . | F | 0.80 | 1.21 |
| Ser | 379 | . | . | . | . | T | T | . | 0.33 | . | . | F | 1.40 | 1.35 |
| Trp | 380 | A | . | . | . | . | T | . | 0.59 | * | * | F | 0.40 | 1.29 |
| Asp | 381 | A | A | . | . | . | . | . | 0.91 | * | . | F | −0.15 | 0.76 |
| Gln | 382 | A | A | . | . | . | . | . | 1.61 | * | . | . | −0.15 | 1.11 |
| Leu | 383 | A | A | . | . | . | . | . | 0.80 | * | . | . | −0.15 | 1.84 |
| Met | 384 | A | A | . | . | . | . | . | 1.10 | * | . | . | 0.30 | 0.91 |
| Arg | 385 | A | A | . | . | . | . | . | 0.58 | * | . | . | 0.30 | 0.87 |
| Gln | 386 | A | A | . | . | . | . | . | 0.27 | * | . | . | −0.30 | 0.87 |
| Leu | 387 | A | A | . | . | . | . | . | 0.31 | * | . | . | 0.45 | 1.27 |
| Asp | 388 | A | A | . | . | . | . | . | 1.12 | * | . | . | 0.75 | 1.30 |
| Leu | 389 | A | A | . | . | . | . | . | 1.72 | * | . | F | 0.60 | 1.21 |
| Thr | 390 | A | . | . | . | . | T | . | 0.72 | * | . | F | 1.30 | 2.54 |
| Lys | 391 | A | . | . | . | . | T | . | 0.72 | . | * | F | 1.30 | 1.07 |
| Asn | 392 | A | . | . | . | . | T | . | 0.68 | * | * | F | 1.30 | 2.16 |
| Glu | 393 | A | . | . | . | . | T | . | −0.18 | * | . | F | 1.30 | 1.11 |
| Ile | 394 | . | . | B | B | . | . | . | 0.74 | * | . | F | 0.75 | 0.41 |
| Asp | 395 | . | . | B | B | . | . | . | 0.47 | * | * | . | 0.60 | 0.50 |
| Val | 396 | . | . | B | B | . | . | . | 0.08 | * | * | . | 0.60 | 0.29 |
| Val | 397 | . | . | B | B | . | . | . | −0.23 | . | . | . | 0.51 | 0.41 |
| Arg | 398 | . | . | B | . | . | T | . | −0.82 | * | . | . | 1.12 | 0.36 |
| Ala | 399 | . | . | B | . | . | T | . | −0.28 | * | . | . | 0.73 | 0.49 |
| Gly | 400 | . | . | . | . | T | T | . | −0.49 | * | . | F | 2.09 | 0.65 |
| Thr | 401 | . | . | . | . | . | T | C | 0.02 | * | * | F | 2.10 | 0.51 |
| Ala | 402 | . | . | . | . | . | . | C | 0.88 | * | * | F | 1.09 | 0.50 |
| Gly | 403 | . | . | . | . | . | T | C | 0.18 | * | * | F | 1.68 | 0.85 |
| Pro | 404 | . | . | . | . | . | T | C | −0.04 | . | . | F | 1.47 | 0.59 |
| Gly | 405 | . | . | . | . | . | T | C | 0.06 | . | . | F | 1.26 | 0.48 |
| Asp | 406 | A | . | . | . | . | T | . | −0.22 | . | . | F | 0.25 | 0.76 |
| Ala | 407 | A | A | . | . | . | . | . | −0.23 | . | . | . | −0.30 | 0.50 |
| Leu | 408 | A | A | . | . | . | . | . | −0.70 | . | . | . | −0.60 | 0.50 |
| Tyr | 409 | A | A | . | . | . | . | . | −1.09 | * | . | . | −0.60 | 0.25 |
| Ala | 410 | A | A | . | . | . | . | . | −0.70 | * | . | . | −0.60 | 0.24 |
| Met | 411 | A | A | . | . | . | . | . | −0.99 | * | . | . | −0.60 | 0.59 |
| Leu | 412 | A | A | . | . | . | . | . | −1.26 | * | . | . | −0.60 | 0.39 |
| Met | 413 | A | A | . | . | . | . | . | −0.44 | * | . | . | −0.60 | 0.29 |
| Lys | 414 | A | A | . | B | . | . | . | −0.16 | * | . | . | −0.60 | 0.47 |
| Trp | 415 | A | A | . | B | . | . | . | 0.12 | * | . | . | 0.15 | 1.14 |
| Val | 416 | A | A | . | B | . | . | . | 0.38 | * | * | . | 0.45 | 1.66 |
| Asn | 417 | A | . | . | . | . | T | . | 1.30 | * | . | F | 1.75 | 0.82 |
| Lys | 418 | A | . | . | . | . | T | . | 1.90 | * | . | F | 2.20 | 1.53 |
| Thr | 419 | . | . | . | . | . | T | C | 1.27 | * | . | F | 3.00 | 3.32 |
| Gly | 420 | . | . | . | . | . | T | C | 1.26 | * | . | F | 2.70 | 2.08 |
| Arg | 421 | . | . | . | . | T | . | . | 1.22 | * | . | F | 2.40 | 1.40 |
| Asn | 422 | . | . | . | . | . | T | C | 1.19 | * | . | F | 1.65 | 0.68 |
| Ala | 423 | . | . | B | . | . | T | . | 0.83 | . | . | . | 1.00 | 0.93 |
| Ser | 424 | . | . | B | . | . | T | . | 0.33 | . | . | . | 0.70 | 0.69 |
| Ile | 425 | . | . | B | . | . | T | . | −0.13 | . | * | . | −0.20 | 0.35 |
| His | 426 | . | A | B | . | . | . | . | −0.24 | . | * | . | −0.60 | 0.29 |
| Thr | 427 | . | A | B | . | . | . | . | −0.83 | * | * | . | −0.60 | 0.36 |
| Leu | 428 | A | A | . | . | . | . | . | −1.06 | * | * | . | −0.60 | 0.52 |
| Leu | 429 | A | A | . | . | . | . | . | −0.76 | * | * | . | −0.60 | 0.31 |
| Asp | 430 | A | A | . | . | . | . | . | 0.24 | * | * | . | −0.30 | 0.38 |
| Ala | 431 | A | A | . | . | . | . | . | −0.32 | * | * | . | 0.30 | 0.89 |
| Leu | 432 | A | A | . | . | . | . | . | −0.01 | * | * | . | 0.75 | 1.07 |
| Glu | 433 | A | A | . | . | . | . | . | 0.80 | * | * | . | 0.75 | 1.11 |
| Arg | 434 | A | A | . | . | . | . | . | 1.72 | * | * | F | 0.90 | 1.90 |
| Met | 435 | A | A | . | . | . | . | . | 1.69 | * | * | F | 0.90 | 4.52 |
| Glu | 436 | A | A | . | . | . | . | . | 1.69 | * | * | F | 0.90 | 3.55 |
| Glu | 437 | A | A | . | . | . | . | . | 2.54 | * | . | F | 0.90 | 1.83 |
| Arg | 438 | A | A | . | . | . | . | . | 2.54 | * | * | F | 0.90 | 3.70 |
| His | 439 | A | A | . | . | . | . | . | 2.48 | * | * | F | 0.90 | 3.70 |
| Ala | 440 | A | A | . | . | . | . | . | 2.19 | * | * | F | 0.90 | 4.28 |
| Lys | 441 | A | A | . | . | . | . | . | 2.19 | * | * | F | 0.90 | 1.53 |
| Glu | 442 | A | A | . | . | . | . | . | 2.19 | * | . | F | 0.90 | 1.95 |
| Lys | 443 | A | A | . | . | . | . | . | 1.27 | * | * | F | 0.90 | 3.22 |
| Ile | 444 | A | A | . | . | . | . | . | 0.49 | * | * | F | 0.90 | 1.33 |
| Gln | 445 | A | A | . | . | . | . | . | 0.22 | * | * | F | 0.75 | 0.63 |
| Asp | 446 | A | A | . | . | . | . | . | 0.18 | * | * | F | −0.15 | 0.23 |
| Leu | 447 | A | A | . | . | . | . | . | −0.12 | * | . | . | −0.30 | 0.56 |
| Leu | 448 | A | A | . | . | . | . | . | −0.51 | * | . | . | 0.55 | 0.43 |
| Val | 449 | A | A | . | . | . | . | . | 0.42 | * | . | F | 0.95 | 0.26 |
| Asp | 450 | A | . | . | . | . | T | . | −0.28 | * | . | F | 1.60 | 0.62 |
| Ser | 451 | . | . | . | . | T | T | . | −1.17 | * | . | F | 2.25 | 0.65 |
| Gly | 452 | . | . | . | . | T | T | . | −0.60 | * | . | F | 2.50 | 0.62 |
| Lys | 453 | . | . | B | . | . | T | . | −0.60 | . | . | F | 1.25 | 0.58 |
| Phe | 454 | . | A | B | . | . | . | . | 0.26 | . | . | . | 0.15 | 0.36 |
| Ile | 455 | . | A | B | . | . | . | . | 0.26 | . | . | . | 0.20 | 0.62 |
| Tyr | 456 | . | A | B | . | . | . | . | 0.21 | . | . | . | 0.55 | 0.52 |
| Leu | 457 | . | A | B | . | . | . | . | 0.24 | . | . | . | −0.03 | 0.59 |
| Glu | 458 | . | A | B | . | . | . | . | −0.14 | . | . | F | 0.54 | 1.22 |
| Asp | 459 | . | A | . | . | T | . | . | 0.26 | . | . | F | 1.66 | 0.77 |
| Gly | 460 | . | . | . | . | T | T | . | 0.56 | . | . | F | 2.78 | 1.26 |
| Thr | 461 | . | . | . | . | . | T | C | −0.06 | * | . | F | 2.70 | 0.73 |
| Gly | 462 | . | . | . | . | . | T | C | 0.46 | * | . | F | 2.13 | 0.33 |
| Ser | 463 | . | . | . | . | . | T | C | −0.36 | . | . | F | 1.26 | 0.44 |
| Ala | 464 | A | . | . | . | . | . | . | −0.36 | . | . | . | 0.14 | 0.25 |
| Val | 465 | . | . | B | . | . | . | . | −0.40 | . | . | . | 0.17 | 0.44 |
| Ser | 466 | . | . | B | . | . | . | . | −0.48 | . | . | . | −0.10 | 0.42 |
| Leu | 467 | . | . | B | . | . | . | . | −0.52 | . | . | . | −0.10 | 0.53 |
| Glu | 468 | A | . | . | . | . | . | . | −0.61 | . | . | . | 0.50 | 0.92 |

In another aspect, the invention provides an antibody that binds a peptide or polypeptide comprising an epitope-bearing portion of a polypeptide described herein. The epitope of this polypeptide portion is an immunogenic or antigenic epitope of a polypeptide of the invention. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Learner, R. A. (1983) Antibodies that react with predetermined sites on proteins. Science 219:660-666. Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides are therefore useful to raise antibodies, including monoclonal antibodies, that bind to a TR4 polypeptide of the invention. See, for instance, Wilson et al., Cell 37:767-778 (1984) at 777. Antigenic epitope-bearing peptides and polypeptides preferably contain a sequence of at least seven, more preferably at least nine and most preferably between at least about 15 to about 30 amino acids contained within the amino acid sequence of SEQ ID NO:1.

Antibodies of the invention may bind one or more antigenic TR4 polypeptides or peptides including, but not limited to: a polypeptide comprising amino acid residues from about 35 to about 92 of SEQ ID NO:1; a polypeptide comprising amino acid residues from about 114 to about 160 of SEQ ID NO:1; a polypeptide comprising amino acid residues from about 169 to about 240 of SEQ ID NO:1; a polypeptide comprising amino acid residues from about 267 to about 298 of SEQ ID NO:1; a polypeptide comprising amino acid residues from about 330 to about 364 of SEQ ID NO:1; a polypeptide comprising amino acid residues from about 391 to about 404 of SEQ ID NO:1; and/or a polypeptide comprising amino acid residues from about 418 to about 465 of SEQ ID NO:1. In this context “about” includes the particularly recited range, larger or smaller by several (5, 4, 3, 2, or 1) amino acids, at either terminus or at both termini. As indicated above, the inventors have determined that the above polypeptide fragments are antigenic regions of the TR4 protein. Epitope-bearing TR4 peptides and polypeptides may be produced by any conventional means. Houghten, R. A., “General method for the rapid solid-phase synthesis of large numbers of peptides: specificity of antigen-antibody interaction at the level of individual amino acids,” Proc. Natl. Acad. Sci. USA 82:5131-5135 (1985). This “Simultaneous Multiple Peptide Synthesis (SMPS)” process is further described in U.S. Pat. No. 4,631,211 to Houghten et al (1986).

As one of skill in the art will appreciate, TR4 polypeptides and the epitope-bearing fragments thereof described herein (e.g., corresponding to a portion of the extracellular domain such as, for example, amino acid residues 1 to 240 of SEQ ID NO:1 can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric TR4 protein or protein fragment alone (Fountoulakis et al., J Biochem 270:3958-3964 (1995)). Thus, antibodies of the invention may bind fusion proteins that comprise all or a portion of a TRAIL receptor polypeptide such as TR4.

Recombinant DNA technology known to those skilled in the art can be used to create novel mutant proteins or “muteins' including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Antibodies of the present invention may also bind such modified TR4 polypeptides or TR4 polypeptide fragments or variants.

For instance, for many proteins, including the extracellular domain of a membrane associated protein or the mature form(s) of a secreted protein, it is known in the art that one or more amino acids may be deleted from the N-terminus or C-terminus without substantial loss of biological function, or loss of the ability to be bound by a specific antibody. For instance, Ron et al., J. Biol. Chem., 268:2984-2988 (1993) reported modified KGF proteins that had heparin binding activity even if 3, 8, or 27 amino-terminal amino acid residues were missing. In the present case, since TR4 is a member of the death domain containing receptor (DDCR) polypeptide family, deletions of N-terminal amino acids up to the cysteine residue at position 109 in SEQ ID NO:1 may retain some biological activity such as the ability to induce apoptosis. Polypeptides having further N-terminal deletions including the cysteine residue at position 109 (C-109) in SEQ ID NO:1 would not be expected to retain such biological activities because this residue is conserved among family members and may be required for forming a disulfide bridge to provide structural stability which is needed for ligand binding.

However, even if deletion of one or more amino acids from the N-terminus of a protein results in modification or loss of one or more biological functions of the protein, other functional activities (e.g., biological activities, ability to multimerize, ability to bind TR4 ligand (e.g., TRAIL)) may still be retained. For example, the ability of shortened TR4 polypeptides to induce and/or bind to antibodies which recognize the complete or mature forms of the TR4 polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a TR4 polypeptide with a large number of deleted N-terminal amino acid residues may retain some biological or immunogenic activities. In fact, peptides composed of as few as six TR4 amino acid residues may often evoke an immune response.

Accordingly, the present invention further provides antibodies that bind polypeptides having one or more residues deleted from the amino terminus of the TR4 amino acid sequence of SEQ ID NO:1 up to the serine residue at position number 463 and polynucleotides encoding such polypeptides. In particular, the present invention provides antibodies that bind polypeptides comprising the amino acid sequence of residues n¹-468 of SEQ ID NO:1, where n¹ is an integer from 2 to 463 corresponding to the position of the amino acid residue in SEQ ID NO:1.

More in particular, the invention provides antibodies that bind polypeptides comprising, or alternatively consisting of, the amino acid sequence of residues of A-2 to E-468; P-3 to E-468; P-4 to E-468; P-5 to E-468; A-6 to E-468; R-7 to E-468; V-8 to E-468; H-9 to E-468; L-10 to E-468; G-1 to E-468; A-12 to E-468; F-13 to E-468; L-14 to E-468; A-15 to E-468; V-16 to E-468; T-17 to E-468; P-18 to E-468; N-19 to E-468; P-20 to E-468; G-21 to E-468; S-22 to E-468; A-23 to E-468; A-24 to E-468; S-25 to E-468; G-26 to E-468; T-27 to E-468; E-28 to E-468; A-29 to E-468; A-30 to E-468; A-31 to E-468; A-32 to E-468; T-33 to E-468; P-34 to E-468; S-35 to E-468; K-36 to E-468; V-37 to E-468; W-38 to E-468; G-39 to E-468; S-40 to E-468; S-41 to E-468; A-42 to E-468; G-43 to E-468; R-44 to E-468; I-45 to E-468; E-46 to E-468; P-47 to E-468; R-48 to E-468; G-49 to E-468; G-50 to E-468; G-51 to E-468; R-52 to E-468; G-53 to E-468; A-54 to E-468; L-55 to E-468; P-56 to E-468; T-57 to E-468; S-58 to E-468; M-59 to E-468; G-60 to E-468; Q-61 to E-468; H-62 to E-468; G-63 to E-468; P-64 to E-468; S-65 to E-468; A-66 to E-468; R-67 to E-468; A-68 to E-468; R-69 to E-468; A-70 to E-468; G-71 to E-468; R-72 to E-468; A-73 to E-468; P-74 to E-468; G-75 to E-468; P-76 to E-468; R-77 to E-468; P-78 to E-468; A-79 to E-468; R-80 to E-468; E-81 to E-468; A-82 to E-468; S-83 to E-468; P-84 to E-468; R-85 to E-468; L-86 to E-468; R-87 to E-468; V-88 to E-468; H-89 to E-468; K-90 to E-468; T-91 to E-468; F-92 to E-468; K-93 to E-468; F-94 to E-468; V-95 to E-468; V-96 to E-468; V-97 to E-468; G-98 to E-468; V-99 to E-468; L-100 to E-468; L-101 to E-468; Q-102 to E-468; V-103 to E-468; V-104 to E-468; P-105 to E-468; S-106 to E-468; S-107 to E-468; A-108 to E-468; A-109 to E-468; T-110 to E-468; I-111 to E-468; K-112 to E-468; L-113 to E-468; H-114 to E-468; D-115 to E-468; Q-116 to E-468; S-117 to E-468; I-118 to E-468; G-119 to E-468; T-120 to E-468; Q-121 to E-468; Q-122 to E-468; W-123 to E-468; E-124 to E-468; H-125 to E-468; S-126 to E-468; P-127 to E-468; L-128 to E-468; G-129 to E-468; E-130 to E-468; L-131 to E-468; C-132 to E-468; P-133 to E-468; P-134 to E-468; G-135 to E-468; S-136 to E-468; H-137 to E-468; R-138 to E-468; S-139 to E-468; E-140 to E-468; R-141 to E-468; P-142 to E-468; G-143 to E-468; A-144 to E-468; C-145 to E-468; N-146 to E-468; R-147 to E-468; C-148 to E-468; T-149 to E-468; E-150 to E-468; G-151 to E-468; V-152 to E-468; G-153 to E-468; Y-154 to E-468; T-155 to E-468; N-156 to E-468; A-157 to E-468; S-158 to E-468; N-159 to E-468; N-160 to E-468; L-161 to E-468; F-162 to E-468; A-163 to E-468; C-164 to E-468; L-165 to E-468; P-166 to E-468; C-167 to E-468; T-168 to E-468; A-169 to E-468; C-170 to E-468; K-171 to E-468; S-172 to E-468; D-173 to E-468; E-174 to E-468; E-175 to E-468; E-176 to E-468; R-177 to E-468; S-178 to E-468; P-179 to E-468; C-180 to E-468; T-181 to E-468; T-182 to E-468; T-183 to E-468; R-184 to E-468; N-185 to E-468; T-186 to E-468; A-187 to E-468; C-188 to E-468; Q-189 to E-468; C-190 to E-468; K-191 to E-468; P-192 to E-468; G-193 to E-468; T-194 to E-468; F-195 to E-468; R-196 to E-468; N-197 to E-468; D-198 to E-468; N-199 to E-468; S-200 to E-468; A-201 to E-468; E-202 to E-468; M-203 to E-468; C-204 to E-468; R-205 to E-468; K-206 to E-468; C-207 to E-468; S-208 to E-468; T-209 to E-468; G-210 to E-468; C-211 to E-468; P-212 to E-468; R-213 to E-468; G-214 to E-468; M-215 to E-468; V-216 to E-468; K-217 to E-468; V-218 to E-468; K-219 to E-468; D-220 to E-468; C-221 to E-468; T-222 to E-468; P-223 to E-468; W-224 to E-468; S-225 to E-468; D-226 to E-468; I-227 to E-468; E-228 to E-468; C-229 to E-468; V-230 to E-468; H-231 to E-468; K-232 to E-468; E-233 to E-468; S-234 to E-468; G-235 to E-468; N-236 to E-468; G-237 to E-468; H-238 to E-468; N-239 to E-468; I-240 to E-468; W-241 to E-468; V-242 to E-468; I-243 to E-468; L-244 to E-468; V-245 to E-468; V-246 to E-468; T-247 to E-468; L-248 to E-468; V-249 to E-468; V-250 to E-468; P-251 to E-468; L-252 to E-468; L-253 to E-468; L-254 to E-468; V-255 to E-468; A-256 to E-468; V-257 to E-468; L-258 to E-468; I-259 to E-468; V-260 to E-468; C-261 to E-468; C-262 to E-468; C-263 to E-468; I-264 to E-468; G-265 to E-468; S-266 to E-468; G-267 to E-468; C-268 to E-468; G-269 to E-468; G-270 to E-468; D-271 to E-468; P-272 to E-468; K-273 to E-468; C-274 to E-468; M-275 to E-468; D-276 to E-468; R-277 to E-468; V-278 to E-468; C-279 to E-468; F-280 to E-468; W-281 to E-468; R-282 to E-468; L-283 to E-468; G-284 to E-468; L-285 to E-468; L-286 to E-468; R-287 to E-468; G-288 to E-468; P-289 to E-468; G-290 to E-468; A-291 to E-468; E-292 to E-468; D-293 to E-468; N-294 to E-468; A-295 to E-468; H-296 to E-468; N-297 to E-468; E-298 to E-468; I-299 to E-468; L-300 to E-468; S-301 to E-468; N-302 to E-468; A-303 to E-468; D-304 to E-468; S-305 to E-468; L-306 to E-468; S-307 to E-468; T-308 to E-468; F-309 to E-468; V-310 to E-468; S-311 to E-468; E-312 to E-468; Q-313 to E-468; Q-314 to E-468; M-315 to E-468; E-316 to E-468; S-317 to E-468; Q-318 to E-468; E-319 to E-468; P-320 to E-468; A-321 to E-468; D-322 to E-468; L-323 to E-468; T-324 to E-468; G-325 to E-468; V-326 to E-468; T-327 to E-468; V-328 to E-468; Q-329 to E-468; S-330 to E-468; P-331 to E-468; G-332 to E-468; E-333 to E-468; A-334 to E-468; Q-335 to E-468; C-336 to E-468; L-337 to E-468; L-338 to E-468; G-339 to E-468; P-340 to E-468; A-341 to E-468; E-342 to E-468; A-343 to E-468; E-344 to E-468; G-345 to E-468; S-346 to E-468; Q-347 to E-468; R-348 to E-468; R-349 to E-468; R-350 to E-468; L-351 to E-468; L-352 to E-468; V-353 to E-468; P-354 to E-468; A-355 to E-468; N-356 to E-468; G-357 to E-468; A-358 to E-468; D-359 to E-468; P-360 to E-468; T-361 to E-468; E-362 to E-468; T-363 to E-468; L-364 to E-468; M-365 to E-468; L-366 to E-468; F-367 to E-468; F-368 to E-468; D-369 to E-468; K-370 to E-468; F-371 to E-468; A-372 to E-468; N-373 to E-468; I-374 to E-468; V-375 to E-468; P-376 to E-468; F-377 to E-468; D-378 to E-468; S-379 to E-468; W-380 to E-468; D-381 to E-468; Q-382 to E-468; L-383 to E-468; M-384 to E-468; R-385 to E-468; Q-386 to E-468; L-387 to E-468; D-388 to E-468; L-389 to E-468; T-390 to E-468; K-391 to E-468; N-392 to E-468; E-393 to E-468; I-394 to E-468; D-395 to E-468; V-396 to E-468; V-397 to E-468; R-398 to E-468; A-399 to E-468; G-400 to E-468; T-401 to E-468; A-402 to E-468; G-403 to E-468; P-404 to E-468; G-405 to E-468; D-406 to E-468; A-407 to E-468; L-408 to E-468; Y-409 to E-468; A-410 to E-468; M-411 to E-468; L-412 to E-468; M-413 to E-468; K-414 to E-468; W-415 to E-468; V-416 to E-468; N-417 to E-468; K-418 to E-468; T-419 to E-468; G-420 to E-468; R-421 to E-468; N-422 to E-468; A-423 to E-468; S-424 to E-468; I-425 to E-468; H-426 to E-468; T-427 to E-468; L-428 to E-468; L-429 to E-468; D-430 to E-468; A-431 to E-468; L-432 to E-468; E-433 to E-468; R-434 to E-468; M-435 to E-468; E-436 to E-468; E-437 to E-468; R-438 to E-468; H-439 to E-468; A-440 to E-468; K-441 to E-468; E-442 to E-468; K-443 to E-468; I-444 to E-468; Q-445 to E-468; D-446 to E-468; L-447 to E-468; L-448 to E-468; V-449 to E-468; D-450 to E-468; S-451 to E-468; G-452 to E-468; K-453 to E-468; F-454 to E-468; I-455 to E-468; Y-456 to E-468; L-457 to E-468; E-458 to E-468; D-459 to E-468; G-460 to E-468; T-461 to E-468; G-462 to E-468; and/or S-463 to E-468 of the TR4 sequence of SEQ ID NO:1.

In another embodiment, N-terminal deletions of the TR4 polypeptide can be described by the general formula n² to 238 where n² is a number from 2 to 238 corresponding to the amino acid sequence identified of SEQ ID NO:1. In specific embodiments, antibodies of the invention bind N terminal deletions of the TR4 comprising, or alternatively consisting of, the amino acid sequence of residues: A-2 to H-238; P-3 to H-238; P-4 to H-238; P-5 to H-238; A-6 to H-238; R-7 to H-238; V-8 to H-238; H-9 to H-238; L-10 to H-238; G-11 to H-238; A-12 to H-238; F-13 to H-238; L-14 to H-238; A-15 to H-238; V-16 to H-238; T-17 to H-238; P-18 to H-238; N-19 to H-238; P-20 to H-238; G-21 to H-238; S-22 to H-238; A-23 to H-238; A-24 to H-238; S-25 to H-238; G-26 to H-238; T-27 to H-238; E-28 to H-238; A-29 to H-238; A-30 to H-238; A-31 to H-238; A-32 to H-238; T-33 to H-238; P-34 to H-238; S-35 to H-238; K-36 to H-238; V-37 to H-238; W-38 to H-238; G-39 to H-238; S-40 to H-238; S-41 to H-238; A-42 to H-238; G-43 to H-238; R-44 to H-238; I-45 to H-238; E-46 to H-238; P-47 to H-238; R-48 to H-238; G-49 to H-238; G-50 to H-238; G-51 to H-238; R-52 to H-238; G-53 to H-238; A-54 to H-238; L-55 to H-238; P-56 to H-238; T-57 to H-238; S-58 to H-238; M-59 to H-238; G-60 to H-238; Q-61 to H-238; H-62 to H-238; G-63 to H-238; P-64 to H-238; S-65 to H-238; A-66 to H-238; R-67 to H-238; A-68 to H-238; R-69 to H-238; A-70 to H-238; G-71 to H-238; R-72 to H-238; A-73 to H-238; P-74 to H-238; G-75 to H-238; P-76 to H-238; R-77 to H-238; P-78 to H-238; A-79 to H-238; R-80 to H-238; E-81 to H-238; A-82 to H-238; S-83 to H-238; P-84 to H-238; R-85 to H-238; L-86 to H-238; R-87 to H-238; V-88 to H-238; H-89 to H-238; K-90 to H-238; T-91 to H-238; F-92 to H-238; K-93 to H-238; F-94 to H-238; V-95 to H-238; V-96 to H-238; V-97 to H-238; G-98 to H-238; V-99 to H-238; L-100 to H-238; L-101 to H-238; Q-102 to H-238; V-103 to H-238; V-104 to H-238; P-105 to H-238; S-106 to H-238; S-107 to H-238; A-108 to H-238; A-109 to H-238; T-110 to H-238; I-111 to H-238; K-112 to H-238; L-113 to H-238; H-114 to H-238; D-115 to H-238; Q-116 to H-238; S-117 to H-238; I-118 to H-238; G-119 to H-238; T-120 to H-238; Q-121 to H-238; Q-122 to H-238; W-123 to H-238; E-124 to H-238; H-125 to H-238; S-126 to H-238; P-127 to H-238; L-128 to H-238; G-129 to H-238; E-130 to H-238; L-131 to H-238; C-132 to H-238; P-133 to H-238; P-134 to H-238; G-135 to H-238; S-136 to H-238; H-137 to H-238; R-138 to H-238; S-139 to H-238; E-140 to H-238; R-141 to H-238; P-142 to H-238; G-143 to H-238; A-144 to H-238; C-145 to H-238; N-146 to H-238; R-147 to H-238; C-148 to H-238; T-149 to H-238; E-150 to H-238; G-151 to H-238; V-152 to H-238; G-153 to H-238; Y-154 to H-238; T-155 to H-238; N-156 to H-238; A-157 to H-238; S-158 to H-238; N-159 to H-238; N-160 to H-238; L-161 to H-238; F-162 to H-238; A-163 to H-238; C-164 to H-238; L-165 to H-238; P-166 to H-238; C-167 to H-238; T-168 to H-238; A-169 to H-238; C-170 to H-238; K-171 to H-238; S-172 to H-238; D-173 to H-238; E-174 to H-238; E-175 to H-238; E-176 to H-238; R-177 to H-238; S-178 to H-238; P-179 to H-238; C-180 to H-238; T-181 to H-238; T-182 to H-238; T-183 to H-238; R-184 to H-238; N-185 to H-238; T-186 to H-238; A-187 to H-238; C-188 to H-238; Q-189 to H-238; C-190 to H-238; K-191 to H-238; P-192 to H-238; G-193 to H-238; T-194 to H-238; F-195 to H-238; R-196 to H-238; N-197 to H-238; D-198 to H-238; N-199 to H-238; S-200 to H-238; A-201 to H-238; E-202 to H-238; M-203 to H-238; C-204 to H-238; R-205 to H-238; K-206 to H-238; C-207 to H-238; S-208 to H-238; T-209 to H-238; G-210 to H-238; C-211 to H-238; P-212 to H-238; R-213 to H-238; G-214 to H-238; M-215 to H-238; V-216 to H-238; K-217 to H-238; V-218 to H-238; K-219 to H-238; D-220 to H-238; C-221 to H-238; T-222 to H-238; P-223 to H-238; W-224 to H-238; S-225 to H-238; D-226 to H-238; I-227 to H-238; E-228 to H-238; C-229 to H-238; V-230 to H-238; H-231 to H-238; K-232 to H-238; and/or E-233 to H-238; of the TR4 extracellular domain sequence of SEQ ID NO:1.

As mentioned above, even if deletion of one or more amino acids from the C-terminus of a protein results in modification of loss of one or more biological functions of the protein, other functional activities (e.g., biological activities, ability to multimerize, ability to bind DR4 ligand (e.g., TRAIL)) may still be retained. For example the ability of the shortened TR4 polypeptide to induce and/or bind to antibodies which recognize the complete or mature forms of the TR4 polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that a TR4 polypeptide with a large number of deleted C-terminal amino acid residues may retain some biological or immunogenic activities. In fact, peptides composed of as few as six TR4 amino acid residues may often evoke an immune response.

Accordingly, the present invention further provides antibodies that bind polypeptides having one or more residues deleted from the carboxy terminus of the amino acid sequence of the TR4 polypeptide sequence of SEQ ID NO:1 up to the alanine residue at position number 30, and polynucleotides encoding such polypeptides. In particular, the present invention provides antibodies that bind polypeptides comprising the amino acid sequence of residues 24-m₁ of SEQ ID NO:1, where m¹ is an integer from 30 to 467 corresponding to the position of the amino acid residue in SEQ ID NO:1.

More in particular, the invention provides antibodies that bind polypeptides comprising, or alternatively consisting of, the amino acid sequence of residues A-24 to L-467; A-24 to S-466; A-24 to V-465; A-24 to A-464; A-24 to S-463; A-24 to G-462; A-24 to T-461; A-24 to G-460; A-24 to D-459; A-24 to E-458; A-24 to L-457; A-24 to Y-456; A-24 to 1455; A-24 to F-454; A-24 to K-453; A-24 to G-452; A-24 to S-451; A-24 to D-450; A-24 to V-449; A-24 to L-448; A-24 to L-447; A-24 to D-446; A-24 to Q-445; A-24 to I-444; A-24 to K-443; A-24 to E-442; A-24 to K-441; A-24 to A-440; A-24 to H-439; A-24 to R-438; A-24 to E-437; A-24 to E-436; A-24 to M-435; A-24 to R-434; A-24 to E-433; A-24 to L-432; A-24 to A-431; A-24 to D-430; A-24 to L-429; A-24 to L-428; A-24 to T-427; A-24 to H-426; A-24 to I-425; A-24 to S-424; A-24 to A-423; A-24 to N-422; A-24 to R-421; A-24 to G-420; A-24 to T-419; A-24 to K-418; A-24 to N-417; A-24 to V-416; A-24 to W-415; A-24 to K-414; A-24 to M-413; A-24 to L-412; A-24 to M-411; A-24 to A-410; A-24 to Y-409; A-24 to L-408; A-24 to A-407; A-24 to D-406; A-24 to G-405; A-24 to P-404; A-24 to G-403; A-24 to A-402; A-24 to T-401; A-24 to G-400; A-24 to A-399; A-24 to R-398; A-24 to V-397; A-24 to V-396; A-24 to D-395; A-24 to I-394; A-24 to E-393; A-24 to N-392; A-24 to K-391; A-24 to T-390; A-24 to L-389; A-24 to D-388; A-24 to L-387; A-24 to Q-386; A-24 to R-385; A-24 to M-384; A-24 to L-383; A-24 to Q-382; A-24 to D-381; A-24 to W-380; A-24 to S-379; A-24 to D-378; A-24 to F-377; A-24 to P-376; A-24 to V-375; A-24 to I-374; A-24 to N-373; A-24 to A-372; A-24 to F-371; A-24 to K-370; A-24 to D-369; A-24 to F-368; A-24 to F-367; A-24 to L-366; A-24 to M-365; A-24 to L-364; A-24 to T-363; A-24 to E-362; A-24 to T-361; A-24 to P-360; A-24 to D-359; A-24 to A-358; A-24 to G-357; A-24 to N-356; A-24 to A-355; A-24 to P-354; A-24 to V-353; A-24 to L-352; A-24 to L-351; A-24 to R-350; A-24 to R-349; A-24 to R-348; A-24 to Q-347; A-24 to S-346; A-24 to G-345; A-24 to E-344; A-24 to A-343; A-24 to E-342; A-24 to A-341; A-24 to P-340; A-24 to G-339; A-24 to L-338; A-24 to L-337; A-24 to C-336; A-24 to Q-335; A-24 to A-334; A-24 to E-333; A-24 to G-332; A-24 to P-331; A-24 to S-330; A-24 to Q-329; A-24 to V-328; A-24 to T-327; A-24 to V-326; A-24 to G-325; A-24 to T-324; A-24 to L-323; A-24 to D-322; A-24 to A-321; A-24 to P-320; A-24 to E-319; A-24 to Q-318; A-24 to S-317; A-24 to E-316; A-24 to M-315; A-24 to Q-314; A-24 to Q-313; A-24 to E-312; A-24 to S-311; A-24 to V-310; A-24 to F-309; A-24 to T-308; A-24 to S-307; A-24 to L-306; A-24 to S-305; A-24 to D-304; A-24 to A-303; A-24 to N-302; A-24 to S-301; A-24 to L-300; A-24 to I-299; A-24 to E-298; A-24 to N-297; A-24 to H-296; A-24 to A-295; A-24 to N-294; A-24 to D-293; A-24 to E-292; A-24 to A-291; A-24 to G-290; A-24 to P-289; A-24 to G-288; A-24 to R-287; A-24 to L-286; A-24 to L-285; A-24 to G-284; A-24 to L-283; A-24 to R-282; A-24 to W-281; A-24 to F-280; A-24 to C-279; A-24 to V-278; A-24 to R-277; A-24 to D-276; A-24 to M-275; A-24 to C-274; A-24 to K-273; A-24 to P-272; A-24 to D-271; A-24 to G-270; A-24 to G-269; A-24 to C-268; A-24 to G-267; A-24 to S-266; A-24 to G-265; A-24 to I-264; A-24 to C-263; A-24 to C-262; A-24 to C-261; A-24 to V-260; A-24 to I-259; A-24 to L-258; A-24 to V-257; A-24 to A-256; A-24 to V-255; A-24 to L-254; A-24 to L-253; A-24 to L-252; A-24 to P-251; A-24 to V-250; A-24 to V-249; A-24 to L-248; A-24 to T-247; A-24 to V-246; A-24 to V-245; A-24 to L-244; A-24 to I-243; A-24 to V-242; A-24 to W-241; A-24 to I-240; A-24 to N-239; A-24 to H-238; A-24 to G-237; A-24 to N-236; A-24 to G-235; A-24 to S-234; A-24 to E-233; A-24 to K-232; A-24 to H-231; A-24 to V-230; A-24 to C-229; A-24 to E-228; A-24 to I-227; A-24 to D-226; A-24 to S-225; A-24 to W-224; A-24 to P-223; A-24 to T-222; A-24 to C-221; A-24 to D-220; A-24 to K-219; A-24 to V-218; A-24 to K-217; A-24 to V-216; A-24 to M-215; A-24 to G-214; A-24 to R-213; A-24 to P-212; A-24 to C-211; A-24 to G-210; A-24 to T-209; A-24 to S-208; A-24 to C-207; A-24 to K-206; A-24 to R-205; A-24 to C-204; A-24 to M-203; A-24 to E-202; A-24 to A-201; A-24 to S-200; A-24 to N-199; A-24 to D-198; A-24 to N-197; A-24 to R-196; A-24 to F-195; A-24 to T-194; A-24 to G-193; A-24 to P-192; A-24 to K-191; A-24 to C-190; A-24 to Q-189; A-24 to C-188; A-24 to A-187; A-24 to T-186; A-24 to N-185; A-24 to R-184; A-24 to T-183; A-24 to T-182; A-24 to T-181; A-24 to C-180; A-24 to P-179; A-24 to S-178; A-24 to R-177; A-24 to E-176; A-24 to E-175; A-24 to E-174; A-24 to D-173; A-24 to S-172; A-24 to K-171; A-24 to C-170; A-24 to A-169; A-24 to T-168; A-24 to C-167; A-24 to P-166; A-24 to L-165; A-24 to C-164; A-24 to A-163; A-24 to F-162; A-24 to L-161; A-24 to N-160; A-24 to N-159; A-24 to S-158; A-24 to A-157; A-24 to N-156; A-24 to T-155; A-24 to Y-154; A-24 to G-153; A-24 to V-152; A-24 to G-151; A-24 to E-150; A-24 to T-149; A-24 to C-148; A-24 to R-147; A-24 to N-146; A-24 to C-145; A-24 to A-144; A-24 to G-143; A-24 to P-142; A-24 to R-141; A-24 to E-140; A-24 to S-139; A-24 to R-138; A-24 to H-137; A-24 to S-136; A-24 to G-135; A-24 to P-134; A-24 to P-133; A-24 to C-132; A-24 to L-131; A-24 to E-130; A-24 to G-129; A-24 to L-128; A-24 to P-127; A-24 to S-126; A-24 to H-125; A-24 to E-124; A-24 to W-123; A-24 to Q-122; A-24 to Q-121; A-24 to T-120; A-24 to G-119; A-24 to I-118; A-24 to S-117; A-24 to Q-116; A-24 to D-115; A-24 to H-114; A-24 to L-113; A-24 to K-112; A-24 to 1-1111; A-24 to T-110; A-24 to A-109; A-24 to A-108; A-24 to S-107; A-24 to S-106; A-24 to P-105; A-24 to V-104; A-24 to V-103; A-24 to Q-102; A-24 to L-101; A-24 to L-100; A-24 to V-99; A-24 to G-98; A-24 to V-97; A-24 to V-96; A-24 to V-95; A-24 to F-94; A-24 to K-93; A-24 to F-92; A-24 to T-91; A-24 to K-90; A-24 to H-89; A-24 to V-88; A-24 to R-87; A-24 to L-86; A-24 to R-85; A-24 to P-84; A-24 to S-83; A-24 to A-82; A-24 to E-81; A-24 to R-80; A-24 to A-79; A-24 to P-78; A-24 to R-77; A-24 to P-76; A-24 to G-75; A-24 to P-74; A-24 to A-73; A-24 to R-72; A-24 to G-71; A-24 to A-70; A-24 to R-69; A-24 to A-68; A-24 to R-67; A-24 to A-66; A-24 to S-65; A-24 to P-64; A-24 to G-63; A-24 to H-62; A-24 to Q-61; A-24 to G-60; A-24 to M-59; A-24 to S-58; A-24 to T-57; A-24 to P-56; A-24 to L-55; A-24 to A-54; A-24 to G-53; A-24 to R-52; A-24 to G-51; A-24 to G-50; A-24 to G-49; A-24 to R-48; A-24 to P-47; A-24 to E-46; A-24 to 145; A-24 to R-44; A-24 to G-43; A-24 to A-42; A-24 to S-41; A-24 to S-40; A-24 to G-39; A-24 to W-38; A-24 to V-37; A-24 to K-36; A-24 to S-35; A-24 to P-34; A-24 to T-33; A-24 to A-32; A-24 to A-31; and/or A-24 to A-30 of the TR4 sequence of SEQ ID NO:1.

In another embodiment, antibodies of the invention bind C-terminal deletions of the TR4 polypeptide that can be described by the general formula 24-m² where m² is a number from 30 to 238 corresponding to the amino acid sequence identified of SEQ ID NO:1. In specific embodiments, the invention provides antibodies that bind TR4 polypeptides comprising, or alternatively consisting of, the amino acid sequence of residues: A-24 to G-237; A-24 to N-236; A-24 to G-235; A-24 to S-234; A-24 to E-233; A-24 to K-232; A-24 to H-231; A-24 to V-230; A-24 to C-229; A-24 to E-228; A-24 to I-227; A-24 to D-226; A-24 to S-225; A-24 to W-224; A-24 to P-223; A-24 to T-222; A-24 to C-221; A-24 to D-220; A-24 to K-219; A-24 to V-218; A-24 to K-217; A-24 to V-216; A-24 to M-215; A-24 to G-214; A-24 to R-213; A-24 to P-212; A-24 to C-211; A-24 to G-210; A-24 to T-209; A-24 to S-208; A-24 to C-207; A-24 to K-206; A-24 to R-205; A-24 to C-204; A-24 to M-203; A-24 to E-202; A-24 to A-201; A-24 to S-200; A-24 to N-199; A-24 to D-198; A-24 to N-197; A-24 to R-196; A-24 to F-195; A-24 to T-194; A-24 to G-193; A-24 to P-192; A-24 to K-191; A-24 to C-190; A-24 to Q-189; A-24 to C-188; A-24 to A-187; A-24 to T-186; A-24 to N-185; A-24 to R-184; A-24 to T-183; A-24 to T-182; A-24 to T-181; A-24 to C-180; A-24 to P-179; A-24 to S-178; A-24 to R-177; A-24 to E-176; A-24 to E-175; A-24 to E-174; A-24 to D-173; A-24 to S-172; A-24 to K-171; A-24 to C-170; A-24 to A-169; A-24 to T-168; A-24 to C-167; A-24 to P-166; A-24 to L-165; A-24 to C-164; A-24 to A-163; A-24 to F-162; A-24 to L-161; A-24 to N-160; A-24 to N-159; A-24 to S-158; A-24 to A-157; A-24 to N-156; A-24 to T-155; A-24 to Y-154; A-24 to G-153; A-24 to V-152; A-24 to G-151; A-24 to E-150; A-24 to T-149; A-24 to C-148; A-24 to R-147; A-24 to N-146; A-24 to C-145; A-24 to A-144; A-24 to G-143; A-24 to P-142; A-24 to R-141; A-24 to E-140; A-24 to S-139; A-24 to R-138; A-24 to H-137; A-24 to S-136; A-24 to G-135; A-24 to P-134; A-24 to P-133; A-24 to C-132; A-24 to L-131; A-24 to E-130; A-24 to G-129; A-24 to L-128; A-24 to P-127; A-24 to S-126; A-24 to H-125; A-24 to E-124; A-24 to W-123; A-24 to Q-122; A-24 to Q-121; A-24 to T-120; A-24 to G-119; A-24 to I-118; A-24 to S-117; A-24 to Q-116; A-24 to D-115; A-24 to H-114; A-24 to L-113; A-24 to K-112; A-24 to I-111; A-24 to T-110; A-24 to A-109; A-24 to A-108; A-24 to S-107; A-24 to S-106; A-24 to P-105; A-24 to V-104; A-24 to V-103; A-24 to Q-102; A-24 to L-101; A-24 to L-100; A-24 to V-99; A-24 to G-98; A-24 to V-97; A-24 to V-96; A-24 to V-95; A-24 to F-94; A-24 to K-93; A-24 to F-92; A-24 to T-91; A-24 to K-90; A-24 to H-89; A-24 to V-88; A-24 to R-87; A-24 to L-86; A-24 to R-85; A-24 to P-84; A-24 to S-83; A-24 to A-82; A-24 to E-81; A-24 to R-80; A-24 to A-79; A-24 to P-78; A-24 to R-77; A-24 to P-76; A-24 to G-75; A-24 to P-74; A-24 to A-73; A-24 to R-72; A-24 to G-71; A-24 to A-70; A-24 to R-69; A-24 to A-68; A-24 to R-67; A-24 to A-66; A-24 to S-65; A-24 to P-64; A-24 to G-63; A-24 to H-62; A-24 to Q-61; A-24 to G-60; A-24 to M-59; A-24 to S-58; A-24 to T-57; A-24 to P-56; A-24 to L-55; A-24 to A-54; A-24 to G-53; A-24 to R-52; A-24 to G-51; A-24 to G-50; A-24 to G-49; A-24 to R-48; A-24 to P-47; A-24 to E-46; A-24 to 145; A-24 to R-44; A-24 to G-43; A-24 to A-42; A-24 to S-41; A-24 to S-40; A-24 to G-39; A-24 to W-38; A-24 to V-37; A-24 to K-36; A-24 to S-35; A-24 to P-34; A-24 to T-33; A-24 to A-32; A-24 to A-31; and/or A-24 to A-30; of the TR4 extracellular domain sequence of SEQ ID NO:1.

The present invention further provides antibodies that bind polypeptides having one or more residues from the carboxy terminus of the amino acid sequence of the TR4 polypeptide of SEQ ID NO:1, up to C-221 of SEQ ID NO:1. In particular, the present invention provides antibodies that bind polypeptides having the amino acid sequence of residues 1-m⁹ of the amino acid sequence in SEQ ID NO:1, where m⁹ is any integer in the range of 221468 and residue C-221 is the position of the first residue from the C-terminus of the complete TR4 polypeptide (shown in SEQ ID NO:1) believed to be required for receptor binding activity of the TR4 protein.

The invention also provides antibodies that bind polypeptides having one or more amino acids deleted from both the amino and the carboxyl termini of a TR4 polypeptide, which may be described generally as having residues n¹-m¹ and/or n²-m² of SEQ ID NO:1, where n¹, n², m¹, and m² are integers as described above.

Also included are antibodies that bind a polypeptide consisting of a portion of the complete TR4 amino acid sequence encoded by the cDNA clone contained in ATCC Deposit No. 97853, where this portion excludes from 1 to about 108 amino acids from the amino terminus of the complete amino acid sequence encoded by the cDNA clone contained in ATCC Deposit No. 97853, or from 1 to about 247 amino acids from the carboxy terminus, or any combination of the above amino terminal and carboxy terminal deletions, of the complete amino acid sequence encoded by the cDNA clone contained in ATCC Deposit No. 97853.

Preferably, antibodies of the present invention bind fragments of TR4 comprising a portion of the extracellular domain; i.e., within residues 24-238 of SEQ ID NO:1, since any portion therein is expected to be soluble.

It will be recognized in the art that some amino acid sequence of TR4 can be varied without significant effect of the structure or function of the protein. If such differences in sequence are contemplated, it should be remembered that there will be critical areas on the protein which determine activity. Such areas will usually comprise residues which make up the ligand binding site or the death domain, or which form tertiary structures which affect these domains.

Thus, the invention further includes antibodies that bind variations of the TR4 protein which show substantial TR4 protein activity or which include regions of TR4 such as the protein fragments discussed below. Such mutants include deletions, insertions, inversions, repeats, and type substitution. Guidance concerning which amino acid changes are likely to be phenotypically silent can be found in Bowie, J. U. et al., Science 247:1306-1310 (1990).

Thus, antibodies of the present invention may bind a fragment, derivative, or analog of the polypeptide of SEQ ID NO:1, or that encoded by the cDNA in ATCC deposit 97853. Such fragments, variants or derivatives may be (i) one in which at least one or more of the amino acid residues are substituted with a conserved or non-conserved amino acid residue (preferably a conserved amino acid residue(s), and more preferably at least one but less than ten conserved amino acid residues) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fe fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Of particular interest are substitutions of charged amino acids with another charged amino acid and with neutral or negatively charged amino acids. The latter results in proteins with reduced positive charge to improve the characteristics of the TR4 protein. The prevention of aggregation is highly desirable. Aggregation of proteins not only results in a loss of activity but can also be problematic when preparing pharmaceutical formulations, because they can be immunogenic. (Pinckard et al., Clin Exp. Immunol. 2:331-340 (1967); Robbins et al., Diabetes 36:838-845 (1987); Cleland et al. Crit. Rev. Therapeutic Drug Carrier Systems 10:307-377 (1993)).

The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the antibodies of the present invention may bind a TR4 receptor that contains one or more amino acid substitutions, deletions or additions, either from natural mutations or human manipulation.

As indicated, changes are preferably of a minor nature, such as conservative amino acid substitutions that do not significantly affect the folding or activity of the protein (see Table 3).

TABLE 3
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

In specific embodiments, the number of substitutions, additions or deletions in the amino acid sequence of SEQ ID NO:1 and/or any of the polypeptide fragments described herein (e.g., the extracellular domain or intracellular domain) is 75, 70, 60, 50, 40, 35, 30, 25, 20, 15, 10, 9, 8, 7, 6, 5, 4, 3, 2, 1 or 30-20, 20-15, 20-10, 15-10, 10-1, 5-10, 1-5, 1-3 or 1-2.

In specific embodiments, the antibodies of the invention bind TR4 polypeptides or fragments or variants thereof (especially a fragment comprising or alternatively consisting of, the extracellular soluble domain of TR4), that contains any one or more of the following conservative mutations in TR4: M1 replaced with A, G, I, L, S, T, or V; A2 replaced with G, I, L, S, T, M, or V; A6 replaced with G, I, L, S, T, M, or V; R7 replaced with H, or K; V8 replaced with A, G, I, L, S, T, or M; H9 replaced with K, or R; L10 replaced with A, G, I, S, T, M, or V; G11 replaced with A, I, L, S, T, M, or V; A12 replaced with G, I, L, S, T, M, or V; F13 replaced with W, or Y; L14 replaced with A, G, I, S, T, M, or V; A15 replaced with G, I, L, S, T, M, or V; V16 replaced with A, G, I, L, S, T, or M; T17 replaced with A, G, I, L, S, M, or V; N19 replaced with Q; G21 replaced with A, I, L, S, T, M, or V; S22 replaced with A, G, I, L, T, M, or V; A23 replaced with G, I, L, S, T, M, or V; A24 replaced with G, I, L, S, T, M, or V; S25 replaced with A, G, I, L, T, M, or V; G26 replaced with A, I, L, S, T, M, or V; I27 replaced with A, G, I, L, S, M, or V; E28 replaced with D; A29 replaced with G, I, L, S, T, M, or V; A30 replaced with G, I, L, S, T, M, or V; A31 replaced with G, I, L, S, T, M, or V; A32 replaced with G, I, L, S, T, M, or V; T33 replaced with A, G, I, L, S, M, or V; S35 replaced with A, G, I, L, T, M, or V; K36 replaced with H, or R; V37 replaced with A, G, I, L, S, T, or M; W38 replaced with F, or Y; G39 replaced with A, I, L, S, T, M, or V; S40 replaced with A, G, I, L, T, M, or V; S41 replaced with A, G, I, L, T, M, or V; A42 replaced with G, I, L, S, T, M, or V; G43 replaced with A, I, L, S, T, M, or V; R44 replaced with H, or K; I45 replaced with A, G, L, S, T, M, or V; E46 replaced with D; R48 replaced with H, or K; G49 replaced with A, I, L, S, T, M, or V; G50 replaced with A, I, L, S, T, M, or V; G51 replaced with A, I, L, S, T, M, or V; R52 replaced with H, or K; G53 replaced with A, I, L, S, T, M, or V; A54 replaced with G, I, L, S, T, M, or V; L55 replaced with A, G, I, S, T, M, or V; T57 replaced with A, G, I, L, S, M, or V; S58 replaced with A, G, I, L, T, M, or V; M59 replaced with A, G, I, L, S, T, or V; G60 replaced with A, I, L, S, T, M, or V; Q61 replaced with N; H62 replaced with K, or R; G63 replaced with A, I, L, S, T, M, or V; S65 replaced with A, G, I, L, T, M, or V; A66 replaced with G, I, L, S, T, M, or V; R67 replaced with H, or K; A68 replaced with G, I, L, S, T, M, or V; R69 replaced with H, or K; A70 replaced with G, I, L, S, T, M, or V; G71 replaced with A, I, L, S, T, M, or V; R72 replaced with H, or K; A73 replaced with G, I, L, S, T, M, or V; G75 replaced with A, I, L, S, T, M, or V; R77 replaced with H, or K; A79 replaced with G, I, L, S, T, M, or V; R80 replaced with H, or K; E81 replaced with D; A82 replaced with G, I, L, S, T, M, or V; S83 replaced with A, G, I, L, T, M, or V; R85 replaced with H, or K; L86 replaced with A, G, I, S, T, M, or V; R87 replaced with H, or K; V88 replaced with A, G, I, L, S, T, or M; H89 replaced with K, or R; K90 replaced with H, or R; T91 replaced with A, G, I, L, S, M, or V; F92 replaced with W, or Y; K93 replaced with H, or R; F94 replaced with W, or Y; V95 replaced with A, G, I, L, S, T, or M; V96 replaced with A, G, I, L, S, T, or M; V97 replaced with A, G, I, L, S, T, or M; G98 replaced with A, I, L, S, T, M, or V; V99 replaced with A, G, I, L, S, T, or M; L100 replaced with A, G, I, S, T, M, or V; L101 replaced with A, G, I, S, T, M, or V; Q102 replaced with N; V103 replaced with A, G, I, L, S, T, or M; V104 replaced with A, G, I, L, S, T, or M; S106 replaced with A, G, I, L, T, M, or V; S107 replaced with A, G, I, L, T, M, or V; A108 replaced with G, I, L, S, T, M, or V; A109 replaced with G, I, L, S, T, M, or V; T110 replaced with A, G, I, L, S, M, or V; I111 replaced with A, G, L, S, T, M, or V; K112 replaced with H, or R; L113 replaced with A, G, I, S, T, M, or V; H114 replaced with K, or R; D115 replaced with E; Q116 replaced with N; S117 replaced with A, G, I, L, T, M, or V; I118 replaced with A, G, L, S, T, M, or V; G119 replaced with A, I, L, S, T, M, or V; T120 replaced with A, G, I, L, S, M, or V; Q121 replaced with N; Q122 replaced with N; W123 replaced with F, or Y; E124 replaced with D; H125 replaced with K, or R; S126 replaced with A, G, I, L, T, M, or V; L128 replaced with A, G, I, S, T, M, or V; G129 replaced with A, I, L, S, T, M, or V; E130 replaced with D; L131 replaced with A, G, I, S, T, M, or V; G135 replaced with A, I, L, S, T, M, or V; S136 replaced with A, G, I, L, T, M, or V; H137 replaced with K, or R; R138 replaced with H, or K; S139 replaced with A, G, I, L, T, M, or V; E140 replaced with D; R141 replaced with H, or K; G143 replaced with A, I, L, S, T, M, or V; A144 replaced with G, I, L, S, T, M, or V; N146 replaced with Q; R147 replaced with H, or K; T149 replaced with A, G, I, L, S, M, or V; E150 replaced with D; G151 replaced with A, I, L, S, T, M, or V; V152 replaced with A, G, I, L, S, T, or M; G153 replaced with A, I, L, S, T, M, or V; Y154 replaced with F, or W; T155 replaced with A, G, I, L, S, M, or V; N156 replaced with Q; A157 replaced with G, I, L, S, T, M, or V; S158 replaced with A, G, I, L, T, M, or V; N159 replaced with Q; N160 replaced with Q; L161 replaced with A, G, I, S, T, M, or V; F162 replaced with W, or Y; A163 replaced with G, I, L, S, T, M, or V; L165 replaced with A, G, I, S, T, M, or V; T168 replaced with A, G, I, L, S, M, or V; A169 replaced with G, I, L, S, T, M, or V; K171 replaced with H, or R; S172 replaced with A, G, I, L, T, M, or V; D173 replaced with E; E174 replaced with D; E175 replaced with D; E176 replaced with D; R177 replaced with H, or K; S178 replaced with A, G, I, L, T, M, or V; T181 replaced with A, G, I, L, S, M, or V; T182 replaced with A, G, I, L, S, M, or V; T183 replaced with A, G, I, L, S, M, or V; R184 replaced with H, or K; N185 replaced with Q; T186 replaced with A, G, I, L, S, M, or V; A187 replaced with G, I, L, S, T, M, or V; Q189 replaced with N; K191 replaced with H, or R; G193 replaced with A, I, L, S, T, M, or V; T194 replaced with A, G, I, L, S, M, or V; F195 replaced with W, or Y; R196 replaced with H, or K; N197 replaced with Q; D198 replaced with E; N199 replaced with Q; S200 replaced with A, G, I, L, T, M, or V; A201 replaced with G, I, L, S, T, M, or V; E202 replaced with D; M203 replaced with A, G, I, L, S, T, or V; R205 replaced with H, or K; K206 replaced with H, or R; S208 replaced with A, G, I, L, T, M, or V; T209 replaced with A, G, I, L, S, M, or V; G210 replaced with A, I, L, S, T, M, or V; R213 replaced with H, or K; G214 replaced with A, I, L, S, T, M, or V; M215 replaced with A, G, I, L, S, T, or V; V216 replaced with A, G, I, L, S, T, or M; K217 replaced with H, or R; V218 replaced with A, G, I, L, S, T, or M; K219 replaced with H, or R; D220 replaced with E; T222 replaced with A, G, I, L, S, M, or V; W224 replaced with F, or Y; S225 replaced with A, G, I, L, T, M, or V; D226 replaced with E; I227 replaced with A, G, L, S, T, M, or V; E228 replaced with D; V230 replaced with A, G, I, L, S, T, or M; H231 replaced with K, or R; K232 replaced with H, or R; E233 replaced with D; S234 replaced with A, G, I, L, T, M, or V; G235 replaced with A, I, L, S, T, M, or V; N236 replaced with Q; G237 replaced with A, I, L, S, T, M, or V; H238 replaced with K, or R; N239 replaced with Q; I240 replaced with A, G, L, S, T, M, or V; W241 replaced with F, or Y; V242 replaced with A, G, I, L, S, T, or M; I243 replaced with A, G, L, S, T, M, or V; L244 replaced with A, G, I, S, T, M, or V; V245 replaced with A, G, I, L, S, T, or M; V246 replaced with A, G, I, L, S, T, or M; T247 replaced with A, G, I, L, S, M, or V; L248 replaced with A, G, I, S, T, M, or V; V249 replaced with A, G, I, L, S, T, or M; V250 replaced with A, G, I, L, S, T, or M; L252 replaced with A, G, I, S, T, M, or V; L253 replaced with A, G, I, S, T, M, or V; L254 replaced with A, G, I, S, T, M, or V; V255 replaced with A, G, I, L, S, T, or M; A256 replaced with G, I, L, S, T, M, or V; V257 replaced with A, G, I, L, S, T, or M; L258 replaced with A, G, I, S, T, M, or V; I259 replaced with A, G, L, S, T, M, or V; V260 replaced with A, G, I, L, S, T, or M; I264 replaced with A, G, L, S, T, M, or V; G265 replaced with A, I, L, S, T, M, or V; S266 replaced with A, G, I, L, T, M, or V; G267 replaced with A, I, L, S, T, M, or V; G269 replaced with A, I, L, S, T, M, or V; G270 replaced with A, I, L, S, T, M, or V; D271 replaced with E; K273 replaced with H, or R; M275 replaced with A, G, I, L, S, T, or V; D276 replaced with E; R277 replaced with H, or K; V278 replaced with A, G, I, L, S, T, or M; F280 replaced with W, or Y; W281 replaced with F, or Y; R282 replaced with H, or K; L283 replaced with A, G, I, S, T, M, or V; G284 replaced with A, I, L, S, T, M, or V; L285 replaced with A, G, I, S, T, M, or V; L286 replaced with A, G, I, S, T, M, or V; R287 replaced with H, or K; G288 replaced with A, I, L, S, T, M, or V; G290 replaced with A, I, L, S, T, M, or V; A291 replaced with G, I, L, S, T, M, or V; E292 replaced with D; D293 replaced with E; N294 replaced with Q; A295 replaced with G, I, L, S, T, M, or V; H296 replaced with K, or R; N297 replaced with Q; E298 replaced with D; I299 replaced with A, G, L, S, T, M, or V; L300 replaced with A, G, I, S, T, M, or V; S301 replaced with A, G, I, L, T, M, or V; N302 replaced with Q; A303 replaced with G, I, L, S, T, M, or V; D304 replaced with E; S305 replaced with A, G, I, L, T, M, or V; L306 replaced with A, G, I, S, T, M, or V; S307 replaced with A, G, I, L, T, M, or V; T308 replaced with A, G, I, L, S, M, or V; F309 replaced with W, or Y; V310 replaced with A, G, I, L, S, T, or M; S311 replaced with A, G, I, L, T, M, or V; E312 replaced with D; Q313 replaced with N; Q314 replaced with N; M315 replaced with A, G, I, L, S, T, or V; E316 replaced with D; S317 replaced with A, G, I, L, T, M, or V; Q318 replaced with N; E319 replaced with D; A321 replaced with G, I, L, S, T, M, or V; D322 replaced with E; L323 replaced with A, G, I, S, T, M, or V; T324 replaced with A, G, I, L, S, M, or V; G325 replaced with A, I, L, S, T, M, or V; V326 replaced with A, G, I, L, S, T, or M; T327 replaced with A, G, I, L, S, M, or V; V328 replaced with A, G, I, L, S, T, or M; Q329 replaced with N; S330 replaced with A, G, I, L, T, M, or V; G332 replaced with A, I, L, S, T, M, or V; E333 replaced with D; A334 replaced with G, I, L, S, T, M, or V; Q335 replaced with N; L337 replaced with A, G, I, S, T, M, or V; L338 replaced with A, G, I, S, T, M, or V; G339 replaced with A, I, L, S, T, M, or V; A341 replaced with G, I, L, S, T, M, or V; E342 replaced with D; A343 replaced with G, L, S, T, M, or V; E344 replaced with D; G345 replaced with A, I, L, S, T, M, or V; S346 replaced with A, G, I, L, T, M, or V; Q347 replaced with N; R348 replaced with H, or K; R349 replaced with H, or K; R350 replaced with H, or K; L351 replaced with A, G, I, S, T, M, or V; L352 replaced with A, G, I, S, T, M, or V; V353 replaced with A, G, I, L, S, T, or M; A355 replaced with G, I, L, S, T, M, or V; N356 replaced with Q; G357 replaced with A, I, L, S, T, M, or V; A358 replaced with G, L, S, T, M, or V; D359 replaced with E; T361 replaced with A, G, I, L, S, M, or V; E362 replaced with D; T363 replaced with A, G, I, L, S, M, or V; L364 replaced with A, G, I, S, T, M, or V; M365 replaced with A, G, I, L, S, T, or V; L366 replaced with A, G, I, S, T, M, or V; F367 replaced with W, or Y; F368 replaced with W, or Y; D369 replaced with E; K370 replaced with H, or R; F371 replaced with W, or Y; A372 replaced with G, I, L, S, T, M, or V; N373 replaced with Q; I374 replaced with A, G, L, S, T, M, or V; V375 replaced with A, G, I, L, S, T, or M; F377 replaced with W, or Y; D378 replaced with E; S379 replaced with A, G, I, L, T, M, or V; W380 replaced with F, or Y; D381 replaced with E; Q382 replaced with N; L383 replaced with A, G, I, S, T, M, or V; M384 replaced with A, G, I, L, S, T, or V; R385 replaced with H, or K; Q386 replaced with N; L387 replaced with A, G, I, S, T, M, or V; D388 replaced with E; L389 replaced with A, G, I, S, T, M, or V; T390 replaced with A, G, I, L, S, M, or V; K391 replaced with H, or R; N392 replaced with Q; E393 replaced with D; I394 replaced with A, G, I, L, S, T, M, or V; D395 replaced with E; V396 replaced with A, G, I, L, S, T, or M; V397 replaced with A, G, I, L, S, T, or M; R398 replaced with H, or K; A399 replaced with G, I, L, S, T, M, or V; G400 replaced with A, I, L, S, T, M, or V; T401 replaced with A, G, I, L, S, M, or V; A402 replaced with G, I, L, S, T, M, or V; G403 replaced with A, I, L, S, T, M, or V; G405 replaced with A, I, L, S, T, M, or V; D406 replaced with E; A407 replaced with G, I, L, S, T, M, or V; L408 replaced with A, G, I, S, T, M, or V; Y409 replaced with F, or W; A410 replaced with G, I, L, S, T, M, or V; M411 replaced with A, G, I, L, S, T, or V; L412 replaced with A, G, I, S, T, M, or V; M413 replaced with A, G, I, L, S, T, or V; K414 replaced with H, or R; W415 replaced with F, or Y; V416 replaced with A, G, I, L, S, T, or M; N417 replaced with Q; K418 replaced with H, or R; T419 replaced with A, G, I, L, S, M, or V; G420 replaced with A, I, L, S, T, M, or V; R421 replaced with H, or K; N422 replaced with Q; A423 replaced with G, I, L, S, T, M, or V; S424 replaced with A, G, I, L, T, M, or V; I425 replaced with A, G, L, S, T, M, or V; H426 replaced with K, or R; T427 replaced with A, G, I, L, S, M, or V; L428 replaced with A, G, I, S, T, M, or V; L429 replaced with A, G, I, S, T, M, or V; D430 replaced with E; A431 replaced with G, I, L, S, T, M, or V; L432 replaced with A, G, I, S, T, M, or V; E433 replaced with D; R434 replaced with H, or K; M435 replaced with A, G, I, L, S, T, or V; E436 replaced with D; E437 replaced with D; R438 replaced with H, or K; H439 replaced with K, or R; A440 replaced with G, I, L, S, T, M, or V; K441 replaced with H, or R; E442 replaced with D; K443 replaced with H, or R; I444 replaced with A, G, L, S, T, M, or V; Q445 replaced with N; D446 replaced with E; L447 replaced with A, G, I, S, T, M, or V; L448 replaced with A, G, I, S, T, M, or V; V449 replaced with A, G, I, L, S, T, or M; D450 replaced with E; S451 replaced with A, G, I, L, T, M, or V; G452 replaced with A, I, L, S, T, M, or V; K453 replaced with H, or R; F454 replaced with W, or Y; I455 replaced with A, G, L, S, T, M, or V; Y456 replaced with F, or W; L457 replaced with A, G, I, S, T, M, or V; E458 replaced with D; D459 replaced with E; G460 replaced with A, I, L, S, T, M, or V; T461 replaced with A, G, I, L, S, M, or V; G462 replaced with A, I, L, S, T, M, or V; S463 replaced with A, G, I, L, T, M, or V; A464 replaced with G, I, L, S, T, M, or V; V465 replaced with A, G, I, L, S, T, or M; S466 replaced with A, G, I, L, T, M, or V; L467 replaced with A, G, I, S, T, M, or V; and/or E468 replaced with D of SEQ ID NO:1.

In specific embodiments, the antibodies of the invention bind TR4 polypeptides or fragments or variants thereof (especially a fragment comprising or alternatively consisting of, the extracellular soluble domain of TR4), that contains any one or more of the following non-conservative mutations in TR4: M1 replaced with D, E, H, K, R, N, Q, F, W, Y, P, or C; A2 replaced with D, E, H, K, R, N, Q, F, W, Y, P, or C; P3 replaced with D, E, H, K, R, A, G, I, L, S, T, M, V, N, Q, F, W, Y, or C; P4 replaced with D, E, H, K, R, A, G, I, L, S, T, M, V, N, Q, F, W, Y, or C; P5 replaced with D, E, H, K, R, A, G, I, L, S, T, M, V, N, Q, F, W, Y, or C; A6 replaced with D, E, H, K, R, N, Q, F, W, Y, P, or C; R7 replaced with D, E, A, G, I, L, S, T, M, V, N, Q, F, W, Y, P, or C; V8 replaced with D, E, H, K, R, N, Q, F, W, Y, P, or C; H9 replaced with D, E, A, G, I, L, S, T, M, V, N, Q, F, W, Y, P, or C; L10 replaced with D, E, H, K, R, N, Q, F, W, Y, P, or C; G11 replaced with D, E, H, K, R, N, Q, F, W, Y, P, or C; A12 replaced with D, E, H, K, R, N, Q, F, W, Y, P, or C; F13 replaced with D, E, H, K, R, N, Q, A, G, I, L, S, T, M, V, P, or C; L14 replaced with D, E, H, K, R, N, Q, F, W, Y, P, or C; A11 replaced with D, E, H, K, R, N, Q, F, W, Y, P, or C; V16 replaced with D, E, H, K, R, N, Q, F, W, Y, P, or C; T17 replaced with D, E, H, K, R, N, Q, F, W, Y, P, or C; P18 replaced with D, E, H, K, R, A, G, I, L, S, T, M, V, N, Q, F, W, Y, or C; N19 replaced with D, E, H, K, R, A, G, I, L, S, T, M, V, F, W, Y, P, or C; P20 replaced with D, E, H, K, R, A, G, I, L, S, T, M, V, N, Q, F, W, Y, or C; G21 replaced with D, E, H, K, R, N, Q, F, W, Y, P, or C; S22 replaced with D, E, H, K, R, N, Q, F, W, Y, P, or C; A23 replaced with D, E, H, K, R, N, Q, F, W, Y, P, or C; A24 replaced with D, E, H, K, R, N, Q, F, W, Y, P, or C; S25 replaced with D, E, H, K, R, N, Q, F, W, Y, P, or C; G26 replaced with D, E, H, K, R, N, Q, F, W, Y, P, or C; T27 replaced with D, E, H, K, R, N, Q, F, W, Y, P, or C; E28 replaced with H, K, R, A, G, I, L, S, T, M, V, N, Q, F, W, Y, P, or C; A29 replaced with D, E, H, K, R, N, Q, F, W, Y, P, or C; A30 replaced with D, E, H, K, R, N, Q, F, W, Y, P, or C; A31 replaced with D, E, H, K, R, N, Q, F, W, Y, P, or C; A32 replaced with D, E, H, K, R, N, Q, F, W, Y, P, or C; T33 replaced with D, E, H, K, R, N, Q, F, W, Y, P, or C; P34 replaced with D, E, H, K, R, A, G, I, L, S, T, M, V, N, Q, F, W, Y, or C; S35 replaced with D, E, H, K, R, N, Q, F, W, Y, P, or C; K36 replaced with D, E, A, G, I, L, S, T, M, V, N, Q, F, W, Y, P, or C; V37 replaced with D, E, H, K, R, N, Q, F, W, Y, P, or C; W38 replaced with D, E, H, K, R, N, Q, A, G, I, L, S, T, M, V, P, or C; G39 replaced with D, E, H, K, R, N, Q, F, W, Y, P, or C; S40 replaced with D, E, H, K, R, N, Q, F, W, Y, P, or C; S41 replaced with D, E, H, K, R, N, Q, F, W, Y, P, or C; A42 replaced with D, E, H, K, R, N, Q, F, W, Y, P, or C; G43 replaced with D, E, H, K, R, N, Q, F, W, Y, P, or C; R44 replaced with D, E, A, G, I, L, S, T, V, N, Q, F, W, Y, P, or C; I45 replaced with D, E, H, K, R, N, Q, F, W, Y, P, or C; E46 replaced with H, K, R, A, G, I, L, S, T, M, V, N, Q, F, W, Y, P, or C; P47 replaced with D, E, H, K, R, A, G, I, L, S, T, M, V, N, Q, F, W, Y, or C; R48 replaced with D, E, A, G, I, L, S, T, M, V, N, Q, F, W, Y, P, or C; G49 replaced with D, E, H, K, R, N, Q, F, W, Y, P, or C; G50 replaced with D, E, H, K, R, N, Q, F, W, Y, P, or C; G51 replaced with D, E, H, K, R, N, Q, F, W, Y, P, or C; R52 replaced with D, E, A, G, I, L, S, T, M, V, N, Q, F, W, Y, P, or C; G53 replaced with D, E, H, K, R, N, Q, F, W, Y, P, or C; A54 replaced with D, E, H, K, R, N, Q, F, W, Y, P, or C; L55 replaced with D, E, H, K, R, N, Q, F, W, Y, P, or C; P56 replaced with D, E, H, K, R, A, G, I, L, S, T, M, V, N, Q, F, W, Y, or C; T57 replaced with D, E, H, K, R, N, Q, F, W, Y, P, or C; S58 replaced with D, E, H, K, R, N, Q, F, W, Y, P, or C; M59 replaced with D, E, H, K, R, N, Q, F, W, Y, P, or C; G60 replaced with D, E, H, K, R, N, Q, F, W, Y, P, or C; Q61 replaced with D, E, H, K, R, A, G, I, L, S, T, M, V, F, W, Y, P, or C; H62 replaced with D, E, A, G, I, L, S, T, M, V, N, Q, F, W, Y, P, or C; G63 replaced with D, E, H, K, R, N, Q, F, W, Y, P, or C; P64 replaced with D, E, H, K, R, A, G, I, L, S, T, M, V, N, Q, F, W, Y, or C; S65 replaced with D, E, H, K, R, N, Q, F, W, Y, P, or C; A66 replaced with D, E, H, K, R, N, Q, F, W, Y, P, or C; R67 replaced with D, E, A, G, I, L, S, T, M, V, N, Q, F, W, Y, P, or C; A68 replaced with D, E, H, K, R, N, Q, F, W, Y, P, or C; R69 replaced with D, E, A, G, I, L, S, T, M, V, N, Q, F, W, Y, P, or C; A70 replaced with D, E, H, K, R, N, Q, F, W, Y, P, or C; G71 replaced with D, E, H, K, R, N, Q, F, W, Y, P, or C; R72 replaced with D, E, A, G, I, L, S, T, M, V, N, Q, F, W, Y, P, or C; A73 replaced with D, E, H, K, R, N, Q, F, W, Y, P, or C; P74 replaced with D, E, H, K, R, A, G, I, L, S, T, M, V, N, Q, F, W, Y, or C; G75 replaced with D, E, H, K, R, N, Q, F, W, Y, P, or C; P76 replaced with D, E, H, K, R, A, G, I, L, S, T, M, V, N, Q, F, W, Y, or C; R77 replaced with D, E, A, G, I, L, S, T, M, V, N, Q, F, W, Y, P, or C; P78 replaced with D, E, H, K, R, A, G, I, L, S, T, M, V, N, Q, F, W, Y, or C; A79 replaced with D, E, H, K, R, N, Q, F, W, Y, P, or C; R80 replaced with D, E, A, G, I, L, S, T, M, V, N, Q, F, W, Y, P, or C; E81 replaced with H, K, R, A, G, I, L, S, T, M, V, N, Q, F, W, Y, P, or C; A82 replaced with D, E, H, K, R, N, Q, F, W, Y, P, or C; S83 replaced with D, E, H, K, R, N, Q, F, W, Y, P, or C; P84 replaced with D, E, H, K, R, A, G, I, L, S, T, M, V, N, Q, F, W, Y, or C; R85 replaced with D, E, A, G, I, L, S, T, M, V, N, Q, F, W, Y, P, or C; L86 replaced with D, E, H, K, R, N, Q, F, W, Y, P, or C; R87 replaced with D, E, A, G, I, L, S, T, M, V, N, Q, F, W, Y, P, or C; V88 replaced with D, E, H, K, R, N, Q, F, W, Y, P, or C; H89 replaced with D, E, A, G, I, L, S, T, M, V, N, Q, F, W, Y, P, or C; K90 replaced with D, E, A, G, I, L, S, T, M, V, N, Q, F, W, Y, P, or C; T91 replaced with D, E, H, K, R, N, Q, F, W, Y, P, or C; F92 replaced with D, E, H, K, R, N, Q, A, G, I, L, S, T, M, V, P, or C; K93 replaced with D, E, A, G, I, L, S, T, M, V, N, Q, F, W, Y, P, or C; F94 replaced with D, E, H, K, R, N, Q, A, G, I, L, S, T, M, V, P, or C; V95 replaced with D, E, H, K, R, N, Q, F, W, Y, P, or C; V96 replaced with D, E, H, K, R, N, Q, F, W, Y, P, or C; V97 replaced with D, E, H, K, R, N, Q, F, W, Y, P, or C; G98 replaced with D, E, H, K, R, N, Q, F, W, Y, P, or C; V99 replaced with D, E, H, K, R, N, Q, F, W, Y, P, or C; L100 replaced with D, E, H, K, R, N, Q, F, W, Y, P, or C; L101 replaced with D, E, H, K, R, N, Q, F, W, Y, P, or C; Q102 replaced with D, E, H, K, R, A, G, I, L, S, T, M, V, F, W, Y, P, or C; V103 replaced with D, E, H, K, R, N, Q, F, W, Y, P, or C; V104 replaced with D, E, H, K, R, N, Q, F, W, Y, P, or C; P105 replaced with D, E, H, K, R, A, G, I, L, S, T, M, V, N, Q, F, W, Y, or C; S106 replaced with D, E, H, K, R, N, Q, F, W, Y, P, or C; S107 replaced with D, E, H, K, R, N, Q, F, W, Y, P, or C; A108 replaced with D, E, H, K, R, N, Q, F, W, Y, P, or C; A109 replaced with D, E, H, K, R, N, Q, F, W, Y, P, or C; T110 replaced with D, E, H, K, R, N, Q, F, W, Y, P, or C; I111 replaced with D, E, H, K, R, N, Q, F, W, Y, P, or C; K112 replaced with D, E, A, G, I, L, S, T, M, V, N, Q, F, W, Y, P, or C; L13 replaced with D, E, H, K, R, N, Q, F, W, Y, P, or C; H14 replaced with D, E, A, G, I, L, S, T, M, V, N, Q, F, W, Y, P, or C; D115 replaced with H, K, R, A, G, I, L, S, T, M, V, N, Q, F, W, Y, P, or C; Q116 replaced with D, E, H, K, R, A, G, I, L, S, T, M, V, F, W, Y, P, or C; S117 replaced with D, E, H, K, R, N, Q, F, W, Y, P, or C; I118 replaced with D, E, H, K, R, N, Q, F, W, Y, P, or C; G119 replaced with D, E, H, K, R, N, Q, F, W, Y, P, or C; T120 replaced with D, E, H, K, R, N, Q, F, W, Y, P, or C; Q121 replaced with D, E, H, K, R, A, G, I, L, S, T, M, V, F, W, Y, P, or C; Q122 replaced with D, E, H, K, R, A, G, L, S, T, M, V, F, W, Y, P, or C; W123 replaced with D, E, H, K, R, N, Q, A, G, I, L, S, T, M, V, P, or C; E124 replaced with H, K, R, A, G, I, L, S, T, M, V, N, Q, F, W, Y, P, or C; H125 replaced with D, E, A, G, I, L, S, T, M, V, N, Q, F, W, Y, P, or C; S126 replaced with D, E, H, K, R, N, Q, F, W, Y, P, or C; P127 replaced with D, E, H, K, R, A, G, I, L, S, T, M, V, N, Q, F, W, Y, or C; L128 replaced with D, E, H, K, R, N, Q, F, W, Y, P, or C; G129 replaced with D, E, H, K, R, N, Q, F, W, Y, P, or C; E130 replaced with H, K, R, A, G, I, L, S, T, M, V, N, Q, F, W, Y, P, or C; L131 replaced with D, E, H, K, R, N, Q, F, W, Y, P, or C; C132 replaced with D, E, H, K, R, A, G, I, L, S, T, M, V, N, Q, F, W, Y, or P; P133 replaced with D, E, H, K, R, A, G, I, L, S, T, M, V, N, Q, F, W, Y, or C; P134 replaced with D, E, H, K, R, A, G, I, L, S, T, M, V, N, Q, F, W, Y, or C; G135 replaced with D, E, H, K, R, N, Q, F, W, Y, P, or C; S136 replaced with D, E, H, K, R, N, Q, F, W, Y, P, or C; H137 replaced with D, E, A, G, I, L, S, T, M, V, N, Q, F, W, Y, P, or C; R138 replaced with D, E, A, G, I, L, S, T, M, V, N, Q, F, W, Y, P, or C; S139 replaced with D, E, H, K, R, N, Q, F, W, Y, P, or C; E140 replaced with H, K, R, A, G, I, L, S, T, M, V, N, Q, F, W, Y, P, or C; R141 replaced with D, E, A, G, I, L, S, T, M, V, N, Q, F, W, Y, P, or C; P142 replaced with D, E, H, K, R, A, G, I, L, S, T, M, V, N, Q, F, W, Y, or C; G143 replaced with D, E, H, K, R, N, Q, F, W, Y, P, or C; A144 replaced with D, E, H, K, R, N, Q, F, W, Y, P, or C; C145 replaced with D, E, H, K, R, A, G, I, L, S, T, M, V, N, Q, F, W, Y, or P; N146 replaced with D, E, H, K, R, A, G, I, L, S, T, M, V, F, W, Y, P, or C; R147 replaced with D, E, A, G, I, L, S, T, M, V, N, Q, F, W, Y, P, or C; C148 replaced with D, E, H, K, R, A, G, I, L, S, T, M, V, N, Q, F, W, Y, or P; T149 replaced with D, E, H, K, R, N, Q, F, W, Y, P, or C; E150 replaced with H, K, R, A, G, I, L, S, T, M, V, N, Q, F, W, Y, P, or C; G151 replaced with D, E, H, K, R, N, Q, F, W, Y, P, or C; V152 replaced with D, E, H, K, R, N, Q, F, W, Y, P, or C; G153 replaced with D, E, H, K, R, N, Q, F, W, Y, P, or C; Y154 replaced with D, E, H, K, R, N, Q, A, G, I, L, S, T, M, V, P, or C; T155 replaced with D, E, H, K, R, N, Q, F, W, Y, P, or C; N156 replaced with D, E, H, K, R, A, G, I, L, S, T, M, V, F, W, Y, P, or C; A157 replaced with D, E, H, K, R, N, Q, F, W, Y, P, or C; S158 replaced with D, E, H, K, R, N, Q, F, W, Y, P, or C; N159 replaced with D, E, H, K, R, A, G, I, L, S, T, M, V, F, W, Y, P, or C; N160 replaced with D, E, H, K, R, A, G, I, L, S, T, M, V, F, W, Y, P, or C; L161 replaced with D, E, H, K, R, N, Q, F, W, Y, P, or C; F162 replaced with D, E, H, K, R, N, Q, A, G, I, L, S, T, M, V, P, or C; A163 replaced with D, E, H, K, R, N, Q, F, W, Y, P, or C; C164 replaced with D, E, H, K, R, A, G, I, L, S, T, M, V, N, Q, F, W, Y, or P; L165 replaced with D, E, H, K, R, N, Q, F, W, Y, P, or C; P166 replaced with D, E, H, K, R, A, G, I, L, S, T, M, V, N, Q, F, W, Y, or C; C167 replaced with D, E, H, K, R, A, G, I, L, S, T, M, V, N, Q, F, W, Y, or P; T168 replaced with D, E, H, K, R, N, Q, F, W, Y, P, or C; A169 replaced with D, E, H, K, R, N, Q, F, W, Y, P, or C; C170 replaced with D, E, H, K, R, A, G, I, L, S, T, M, V, N, Q, F, W, Y, or P; K171 replaced with D, E, A, G, I, L, S, T, M, V, N, Q, F, W, Y, P, or C; S172 replaced with D, E, H, K, R, N, Q, F, W, Y, P, or C; D173 replaced with H, K, R, A, G, I, L, S, T, M, V, N, Q, F, W, Y, P, or C; E174 replaced with H, K, R, A, G, I, L, S, T, M, V, N, Q, F, W, Y, P, or C; E175 replaced with H, K, R, A, G, I, L, S, T, M, V, N, Q, F, W, Y, P, or C; E176 replaced with H, K, R, A, G, I, L, S, T, M, V, N, Q, F, W, Y, P, or C; R177 replaced with D, E, A, G, I, L, S, T, M, V, N, Q, F, W, Y, P, or C; S178 replaced with D, E, H, K, R, N, Q, F, W, Y, P, or C; P179 replaced with D, E, H, K, R, A, G, I, L, S, T, M, V, N, Q, F, W, Y, or C; C180 replaced with D, E, H, K, R, A, G, I, L, S, T, M, V, N, Q, F, W, Y, or P; T181 replaced with D, E, H, K, R, N, Q, F, W, Y, P, or C; T182 replaced with D, E, H, K, R, N, Q, F, W, Y, P, or C; T183 replaced with D, E, H, K, R, N, Q, F, W, Y, P, or C; R184 replaced with D, E, A, G, I, L, S, T, M, V, N, Q, F, W, Y, P, or C; N185 replaced with D, E, H, K, R, A, G, I, L, S, T, M, V, F, W, Y, P, or C; T186 replaced with D, E, H, K, R, N, Q, F, W, Y, P, or C; A187 replaced with D, E, H, K, R, N, Q, F, W, Y, P, or C; C188 replaced with D, E, H, K, R, A, G, I, L, S, T, M, V, N, Q, F, W, Y, or P; Q189 replaced with D, E, H, K, R, A, G, I, L, S, T, M, V, F, W, Y, P, or C; C190 replaced with D, E, H, K, R, A, G, I, L, S, T, M, V, N, Q, F, W, Y, or P; K191 replaced with D, E, A, G, I, L, S, T, M, V, N, Q, F, W, Y, P, or C; P192 replaced with D, E, H, K, K, A, G, I, L, S, T, M, V, N, Q, F, W, Y, or C; G193 replaced with D, E, H, K, R, N, Q, F, W, Y, P, or C; T194 replaced with D, E, H, K, R, N, Q, F, W, Y, P, or C; F195 replaced with D, E, H, K, R, N, Q, A, G, I, L, S, T, M, V, P, or C; R196 replaced with D, E, A, G, I, L, S, T, M, V, N, Q, F, W, Y, P, or C; N197 replaced with D, E, H, K, R, A, G, I, L, S, T, M, V, F, W, Y, P, or C; D198 replaced with H, K, R, A, G, I, L, S, T, M, V, N, Q, F, W, Y, P, or C; N199 replaced with D, E, H, K, R, A, G, I, L, S, T, M, V, F, W, Y, P, or C; S200 replaced with D, E, H, K, R, N, Q, F, W, Y, P, or C; A201 replaced with D, E, H, K, R, N, Q, F, W, Y, P, or C; E202 replaced with H, K, R, A, G, I, L, S, T, M, V, N, Q, F, W, Y, P, or C; M203 replaced with D, E, H, K, R, N, Q, F, W, Y, P, or C; C204 replaced with D, E, H, K, R, A, G, I, L, S, T, M, V, N, Q, F, W, Y, or P; R205 replaced with D, E, A, G, I, L, S, T, M, V, N, Q, F, W, Y, P, or C; K206 replaced with D, E, A, G, I, L, S, T, M, V, N, Q, F, W, Y, P, or C; C207 replaced with D, E, H, K, R, A, G, I, L, S, T, M, V, N, Q, F, W, Y, or P; S208 replaced with D, E, H, K, R, N, Q, F, W, Y, P, or C; T209 replaced with D, E, H, K, R, N, Q, F, W, Y, P, or C; G210 replaced with D, E, H, K, R, N, Q, F, W, Y, P, or C; C211 replaced with D, E, H, K, R, A, G, I, L, S, T, M, V, N, Q, F, W, Y, or P; P212 replaced with D, E, H, K, R, A, G, I, L, S, T, M, V, N, Q, F, W, Y, or C; R213 replaced with D, E, A, G, I, L, S, T, M, V, N, Q, F, W, Y, P, or C; G214 replaced with D, E, H, K, R, N, Q, F, W, Y, P, or C; M215 replaced with D, E, H, K, R, N, Q, F, W, Y, P, or C; V216 replaced with D, E, H, K, R, N, Q, F, W, Y, P, or C; K217 replaced with D, E, A, G, I, L, S, T, M, V, N, Q, F, W, Y, P, or C; V218 replaced with D, E, H, K, R, N, Q, F, W, Y, P, or C; K219 replaced with D, E, A, G, I, L, S, T, M, V, N, Q, F, W, Y, P, or C; D220 replaced with H, K, R, A, G, I, L, S, T, V, N, Q, F, W, Y, P, or C; C221 replaced with D, E, H, K, R, A, G, I, L, S, T, M, V, N, Q, F, W, Y, or P; T222 replaced with D, E, H, K, R, N, Q, F, W, Y, P, or C; P223 replaced with D, E, H, K, R, A, G, I, L, S, T, M, V, N, Q, F, W, Y, or C; W224 replaced with D, E, H, K, R, N, Q, A, G, I, L, S, T, M, V, P, or C; S225 replaced with D, E, H, K, R, N, Q, F, W, Y, P, or C; D226 replaced with H, K, R, A, G, I, L, S, T, M, V, N, Q, F, W, Y, P, or C; I227 replaced with D, E, H, K, R, N, Q, F, W, Y, P, or C; E228 replaced with H, K, R, A, G, I, L, S, T, M, V, N, Q, F, W, Y, P, or C; C229 replaced with D, E, H, K, R, A, G, I, L, S, T, M, V, N, Q, F, W, Y, or P; V230 replaced with D, E, H, K, R, N, Q, F, W, Y, P, or C; H231 replaced with D, E, A, G, I, L, S, T, M, V, N, Q, F, W, Y, P, or C; K232 replaced with D, E, A, G, I, L, S, T, M, V, N, Q, F, W, Y, P, or C; E233 replaced with H, K, R, A, G, I, L, S, T, M, V, N, Q, F, W, Y, P, or C; S234 replaced with D, E, H, K, R, N, Q, F, W, Y, P, or C; G235 replaced with D, E, H, K, R, N, Q, F, W, Y, P, or C; N236 replaced with D, E, H, K, R, A, G, I, L, S, T, M, V, F, W, Y, P, or C; G237 replaced with D, E, H, K, R, N, Q, F, W, Y, P, or C; H238 replaced with D, E, A, G, I, L, S, T, M, V, N, Q, F, W, Y, P, or C; N239 replaced with D, E, H, K, R, A, G, I, L, S, T, M, V, F, W, Y, P, or C; I240 replaced with D, E, H, K, R, N, Q, F, W, Y, P, or C; W241 replaced with D, E, H, K, R, N, Q, A, G, I, L, S, T, M, V, P, or C; V242 replaced with D, E, H, K, R, N, Q, F, W, Y, P, or C; I243 replaced with D, E, H, K, R, N, Q, F, W, Y, P, or C; L244 replaced with D, E, H, K, R, N, Q, F, W, Y, P, or C; V245 replaced with D, E, H, K, R, N, Q, F, W, Y, P, or C; V246 replaced with D, E, H, K, R, N, Q, F, W, Y, P, or C; T247 replaced with D, E, H, K, R, N, Q, F, W, Y, P, or C; L248 replaced with D, E, H, K, R, N, Q, F, W, Y, P, or C; V249 replaced with D, E, H, K, R, N, Q, F, W, Y, P, or C; V250 replaced with D, E, H, K, R, N, Q, F, W, Y, P, or C; P251 replaced with D, E, H, K, R, A, G, I, L, S, T, M, V, N, Q, F, W, Y, or C; L252 replaced with D, E, H, K, R, N, Q, F, W, Y, P, or C; L253 replaced with D, E, H, K, R, N, Q, F, W, Y, P, or C; L254 replaced with D, E, H, K, R, N, Q, F, W, Y, P, or C; V255 replaced with D, E, H, K, R, N, Q, F, W, Y, P, or C; A256 replaced with D, E, H, K, R, N, Q, F, W, Y, P, or C; V257 replaced with D, E, H, K, R, N, Q, F, W, Y, P, or C; L258 replaced with D, E, H, K, R, N, Q, F, W, Y, P, or C; I259 replaced with D, E, H, K, R, N, Q, F, W, Y, P, or C; V260 replaced with D, E, H, K, R, N, Q, F, W, Y, P, or C; C261 replaced with D, E, H, K, R, A, G, I, L, S, T, M, V, N, Q, F, W, Y, or P; C262 replaced with D, E, H, K, R, A, G, I, L, S, T, M, V, N, Q, F, W, Y, or P; C263 replaced with D, E, H, K, R, A, G, I, L, S, T, M, V, N, Q, F, W, Y, or P; I264 replaced with D, E, H, K, R, N, Q, F, W, Y, P, or C; G265 replaced with D, E, H, K, R, N, Q, F, W, Y, P, or C; S266 replaced with D, E, H, K, R, N, Q, F, W, Y, P, or C; G267 replaced with D, E, H, K, R, N, Q, F, W, Y, P, or C; C268 replaced with D, E, H, K, R, A, G, I, L, S, T, M, V, N, Q, F, W, Y, or P; G269 replaced with D, E, H, K, R, N, Q, F, W, Y, P, or C; G270 replaced with D, E, H, K, R, N, Q, F, W, Y, P, or C; D271 replaced with H, K, R, A, G, I, L, S, T, M, V, N, Q, F, W, Y, P, or C; P272 replaced with D, E, H, K, R, A, G, I, L, S, T, M, V, N, Q, F, W, Y, or C; K273 replaced with D, E, A, G, I, L, S, T, M, V, N, Q, F, W, Y, P, or C; C274 replaced with D, E, H, K, R, A, G, I, L, S, T, M, V, N, Q, F, W, Y, or P; M275 replaced with D, E, H, K, R, N, Q, F, W, Y, P, or C; D276 replaced with H, K, R, A, G, I, L, S, T, M, V, N, Q, F, W, Y, P, or C; R277 replaced with D, E, A, G, I, L, S, T, M, V, N, Q, F, W, Y, P, or C; V278 replaced with D, E, H, K, R, N, Q, F, W, Y, P, or C; C279 replaced with D, E, H, K, R, A, G, I, L, S, T, M, V, N, Q, F, W, Y, or P; F280 replaced with D, E, H, K, R, N, Q, A, G, I, L, S, T, M, V, P, or C; W281 replaced with D, E, H, K, R, N, Q, A, G, I, L, S, T, M, V, P, or C; R282 replaced with D, E, A, G, I, L, S, T, M, V, N, Q, F, W, Y, P, or C; L283 replaced with D, E, H, K, R, N, Q, F, W, Y, P, or C; G284 replaced with D, E, H, K, R, N, Q, F, W, Y, P, or C; L285 replaced with D, E, H, K, R, N, Q, F, W, Y, P, or C; L286 replaced with D, E, H, K, R, N, Q, F, W, Y, P, or C; R287 replaced with D, E, A, G, I, L, S, T, M, V, N, Q, F, W, Y, P, or C; G288 replaced with D, E, H, K, R, N, Q, F, W, Y, P, or C; P289 replaced with D, E, H, K, R, A, G, I, L, S, T, M, V, N, Q, F, W, Y, or C; G290 replaced with D, E, H, K, R, N, Q, F, W, Y, P, or C; A291 replaced with D, E, H, K, R, N, Q, F, W, Y, P, or C; E292 replaced with H, K, R, A, G, I, L, S, T, M, V, N, Q, F, W, Y, P, or C; D293 replaced with H, K, R, A, G, I, L, S, T, M, V, N, Q, F, W, Y, P, or C; N294 replaced with D, E, H, K, R, A, G, I, L, S, T, M, V, F, W, Y, P, or C; A295 replaced with D, E, H, K, R, N, Q, F, W, Y, P, or C; H296 replaced with D, E, A, G, I, L, S, T, M, V, N, Q, F, W, Y, P, or C; N297 replaced with D, E, H, K, R, A, G, I, L, S, T, M, V, F, W, Y, P, or C; E298 replaced with H, K, R, A, G, I, L, S, T, M, V, N, Q, F, W, Y, P, or C; I299 replaced with D, E, H, K, R, N, Q, F, W, Y, P, or C; L300 replaced with D, E, H, K, R, N, Q, F, W, Y, P, or C; S301 replaced with D, E, H, K, R, N, Q, F, W, Y, P, or C; N302 replaced with D, E, H, K, R, A, G, I, L, S, T, M, V, F, W, Y, P, or C; A303 replaced with D, E, H, K, R, N, Q, F, W, Y, P, or C; D304 replaced with H, K, R, A, G, I, L, S, T, M, V, N, Q, F, W, Y, P, or C; S305 replaced with D, E, H, K, R, N, Q, F, W, Y, P, or C; L306 replaced with D, E, H, K, R, N, Q, F, W, Y, P, or C; S307 replaced with D, E, H, K, R, N, Q, F, W, Y, P, or C; T308 replaced with D, E, H, K, R, N, Q, F, W, Y, P, or C; F309 replaced with D, E, H, K, R, N, Q, A, G, I, L, S, T, M, V, P, or C; V310 replaced with D, E, H, K, R, N, Q, F, W, Y, P, or C; S311 replaced with D, E, H, K, R, N, Q, F, W, Y, P, or C; E312 replaced with H, K, R, A, G, I, L, S, T, M, V, N, Q, F, W, Y, P, or C; Q313 replaced with D, E, H, K, R, A, G, I, L, S, T, M, V, F, W, Y, P, or C; Q314 replaced with D, E, H, K, R, A, G, I, L, S, T, M, V, F, W, Y, P, or C; M315 replaced with D, E, H, K, R, N, Q, F, W, Y, P, or C; E316 replaced with H, K, R, A, G, I, L, S, T, M, V, N, Q, F, W, Y, P, or C; S317 replaced with D, E, H, K, R, N, Q, F, W, Y, P, or C; Q318 replaced with D, E, H, K, R, A, G, I, L, S, T, M, V, F, W, Y, P, or C; E319 replaced with H, K, R, A, G, I, L, S, T, M, V, N, Q, F, W, Y, P, or C; P320 replaced with D, E, H, K, R, A, G, I, L, S, T, M, V, N, Q, F, W, Y, or C; A321 replaced with D, E, H, K, R, N, Q, F, W, Y, P, or C; D322 replaced with H, K, R, A, G, I, L, S, T, M, V, N, Q, F, W, Y, P, or C; L323 replaced with D, E, H, K, R, N, Q, F, W, Y, P, or C; T324 replaced with D, E, H, K, R, N, Q, F, W, Y, P, or C; G325 replaced with D, E, H, K, R, N, Q, F, W, Y, P, or C; V326 replaced with D, E, H, K, R, N, Q, F, W, Y, P, or C; T327 replaced with D, E, H, K, R, N, Q, F, W, Y, P, or C; V328 replaced with D, E, H, K, R, N, Q, F, W, Y, P, or C; Q329 replaced with D, E, H, K, R, A, G, I, L, S, T, M, V, F, W, Y, P, or C; S330 replaced with D, E, H, K, R, N, Q, F, W, Y, P, or C; P331 replaced with D, E, H, K, R, A, G, I, L, S, T, M, V, N, Q, F, W, Y, or C; G332 replaced with D, E, H, K, R, N, Q, F, W, Y, P, or C; E333 replaced with H, K, R, A, G, I, L, S, T, M, V, N, Q, F, W, Y, P, or C; A334 replaced with D, E, H, K, R, N, Q, F, W, Y, P, or C; Q335 replaced with D, E, H, K, R, A, G, I, L, S, T, M, V, F, W, Y, P, or C; C336 replaced with D, E, H, K, R, A, G, I, L, S, T, M, V, N, Q, F, W, Y, or P; L337 replaced with D, E, H, K, R, N, Q, F, W, Y, P, or C; L338 replaced with D, E, H, K, R, N, Q, F, W, Y, P, or C; G339 replaced with D, E, H, K, R, N, Q, F, W, Y, P, or C; P340 replaced with D, E, H, K, R, A, G, I, L, S, T, M, V, N, Q, F, W, Y, or C; A341 replaced with D, E, H, K, R, N, Q, F, W, Y, P, or C; E342 replaced with H, K, R, A, G, I, L, S, T, M, V, N, Q, F, W, Y, P, or C; A343 replaced with D, E, H, K, R, N, Q, F, W, Y, P, or C; E344 replaced with H, K, R, A, G, I, L, S, T, M, V, N, Q, F, W, Y, P, or C; G345 replaced with D, E, H, K, R, N, Q, F, W, Y, P, or C; S346 replaced with D, E, H, K, R, N, Q, F, W, Y, P, or C; Q347 replaced with D, E, H, K, R, A, G, I, L, S, T, M, V, F, W, Y, P, or C; R348 replaced with D, E, A, G, I, L, S, T, M, V, N, Q, F, W, Y, P, or C; R349 replaced with D, E, A, G, I, L, S, T, M, V, N, Q, F, W, Y, P, or C; R350 replaced with D, E, A, G, I, L, S, T, M, V, N, Q, F, W, Y, P, or C; L351 replaced with D, E, H, K, R, N, Q, F, W, Y, P, or C; L352 replaced with D, E, H, K, R, N, Q, F, W, Y, P, or C; V353 replaced with D, E, H, K, R, N, Q, F, W, Y, P, or C; P354 replaced with D, E, H, K, R, A, G, I, L, S, T, M, V, N, Q, F, W, Y, or C; A355 replaced with D, E, H, K, P, N, Q, F, W, Y, P, or C; N356 replaced with D, E, H, K, R, A, G, I, L, S, T, M, V, F, W, Y, P, or C; G357 replaced with D, E, H, K, R, N, Q, F, W, Y, P, or C; A358 replaced with D, E, H, K, R, N, Q, F, W, Y, P, or C; D359 replaced with H, K, R, A, G, I, L, S, T, M, V, N, Q, F, W, Y, P, or C; P360 replaced with D, E, H, K, R, A, G, I, L, S, T, M, V, N, Q, F, W, Y, or C; T361 replaced with D, E, H, K, R, N, Q, F, W, Y, P, or C; E362 replaced with H, K, R, A, G, I, L, S, T, M, V, N, Q, F, W, Y, P, or C; T363 replaced with D, E, H, K, R, N, Q, F, W, Y, P, or C; L364 replaced with D, E, H, K, R, N, Q, F, W, Y, P, or C; M365 replaced with D, E, H, K, R, N, Q, F, W, Y, P, or C; L366 replaced with D, E, H, K, R, N, Q, F, W, Y, P, or C; F367 replaced with D, E, H, K, R, N, Q, A, G, I, L, S, T, M, V, P, or C; F368 replaced with D, E, H, K, R, N, Q, A, G, I, L, S, T, M, V, P, or C; D369 replaced with H, K, R, A, G, I, L, S, T, M, V, N, Q, F, W, Y, P, or C; K370 replaced with D, E, A, G, I, L, S, T, M, V, N, Q, F, W, Y, P, or C; F371 replaced with D, E, H, K, R, N, Q, A, G, I, L, S, T, M, V, P, or C; A372 replaced with D, E, H, K, R, N, Q, F, W, Y, P, or C; N373 replaced with D, E, H, K, R, A, G, I, L, S, T, M, V, F, W, Y, P, or C; I374 replaced with D, E, H, K, R, N, Q, F, W, Y, P, or C; V375 replaced with D, E, H, K, R, N, Q, F, W, Y, P, or C; P376 replaced with D, E, H, K, R, A, G, I, L, S, T, M, V, N, Q, F, W, Y, or C; F377 replaced with D, E, H, K, R, N, Q, A, G, I, L, S, T, M, V, P, or C; D378 replaced with H, K, R, A, G, I, L, S, T, M, V, N, Q, F, W, Y, P, or C; S379 replaced with D, E, H, K, K, R, N, Q, F, W, Y, P, or C; W380 replaced with D, E, H, K, R, N, Q, A, G, I, L, S, T, M, V, P, or C; D381 replaced with H, K, R, A, G, I, L, S, T, M, V, N, Q, F, W, Y, P, or C; Q382 replaced with D, E, H, K, R, A, G, I, L, S, T, M, V, F, W, Y, P, or C; L383 replaced with D, E, H, K, R, N, Q, F, W, Y, P, or C; M384 replaced with D, E, H, K, R, N, Q, F, W, Y, P, or C; R385 replaced with D, E, A, G, I, L, S, T, M, V, N, Q, F, W, Y, P, or C; Q386 replaced with D, E, H, K, R, A, G, I, L, S, T, M, V, F, W, Y, P, or C; L387 replaced with D, E, H, K, R, N, Q, F, W, Y, P, or C; D388 replaced with H, K, R, A, G, I, L, S, T, M, V, N, Q, F, W, Y, P, or C; L389 replaced with D, E, H, K, R, N, Q, F, W, Y, P, or C; T390 replaced with D, E, H, K, R, N, Q, F, W, Y, P, or C; K391 replaced with D, E, A, G, I, L, S, T, M, V, N, Q, F, W, Y, P, or C; N392 replaced with D, E, H, K, R, A, G, I, L, S, T, M, V, F, W, Y, P, or C; E393 replaced with H, K, R, A, G, I, L, S, T, M, V, N, Q, F, W, Y, P, or C; I394 replaced with D, E, H, K, R, N, Q, F, W, Y, P, or C; D395 replaced with H, K, R, A, G, I, L, S, T, M, V, N, Q, F, W, Y, P, or C; V396 replaced with D, E, H, K, R, N, Q, F, W, Y, P, or C; V397 replaced with D, E, H, K, R, N, Q, F, W, Y, P, or C; R398 replaced with D, E, A, G, I, L, S, T, M, V, N, Q, F, W, Y, P, or C; A399 replaced with D, E, H, K, R, N, Q, F, W, Y, P, or C; G400 replaced with D, E, H, K, R, N, Q, F, W, Y, P, or C; T401 replaced with D, E, H, K, R, N, Q, F, W, Y, P, or C; A402 replaced with D, E, H, K, R, N, Q, F, W, Y, P, or C; G403 replaced with D, E, H, K, R, N, Q, F, W, Y, P, or C; P404 replaced with D, E, H, K, R, A, G, I, L, S, T, M, V, N, Q, F, W, Y, or C; G405 replaced with D, E, H, K, R, N, Q, F, W, Y, P, or C; D406 replaced with H, K, R, A, G, I, L, S, T, M, V, N, Q, F, W, Y, P, or C; A407 replaced with D, E, H, K, R, N, Q, F, W, Y, P, or C; L408 replaced with D, E, H, K, R, N, Q, F, W, Y, P, or C; Y409 replaced with D, E, H, K, R, N, Q, A, G, I, L, S, T, M, V, P, or C; A410 replaced with D, E, H, K, R, N, Q, F, W, Y, P, or C; M411 replaced with D, E, H, K, R, N, Q, F, W, Y, P, or C; L412 replaced with D, E, H, K, R, N, Q, F, W, Y, P, or C; M413 replaced with D, E, H, K, R, N, Q, F, W, Y, P, or C; K414 replaced with D, E, A, G, I, L, S, T, M, V, N, Q, F, W, Y, P, or C; W415 replaced with D, E, H, K, R, N, Q, A, G, I, L, S, T, M, V, P, or C; V416 replaced with D, E, H, K, R, N, Q, F, W, Y, P, or C; N417 replaced with D, E, F, K, R, A, G, I, L, S, T, M, V, F, W, Y, P, or C; K418 replaced with D, E, A, G, I, L, S, T, M, V, N, Q, F, W, Y, P, or C; T419 replaced with D, E, H, K, R, N, Q, F, W, Y, P, or C; G420 replaced with D, E, H, K, R, N, Q, F, W, Y, P, or C; R421 replaced with D, E, A, G, I, L, S, T, M, V, N, Q, F, W, Y, P, or C; N422 replaced with D, E, H, K, R, A, G, I, L, S, T, M, V, F, W, Y, P, or C; A423 replaced with D, E, H, K, R, N, Q, F, W, Y, P, or C; S424 replaced with D, E, H, K, R, N, Q, F, W, Y, P, or C; I425 replaced with D, E, H, K, R, N, Q, F, W, Y, P, or C; H426 replaced with D, E, A, G, I, L, S, T, M, V, N, Q, F, W, Y, P, or C; T427 replaced with D, E, H, K, R, N, Q, F, W, Y, P, or C; L428 replaced with D, E, H, K, R, N, Q, F, W, Y, P, or C; L429 replaced with D, E, H, K, R, N, Q, F, W, Y, P, or C; D430 replaced with H, K, R, A, G, I, L, S, T, M, V, N, Q, F, W, Y, P, or C; A431 replaced with D, E, H, K, R, N, Q, F, W, Y, P, or C; L432 replaced with D, E, H, K, R, N, Q, F, W, Y, P, or C; E433 replaced with H, K, R, A, G, I, L, S, T, M, V, N, Q, F, W, Y, P, or C; R434 replaced with D, E, A, G, I, L, S, T, M, V, N, Q, F, W, Y, P, or C; M435 replaced with D, E, H, K, R, N, Q, F, W, Y, P, or C; E436 replaced with H, K, R, A, G, I, L, S, T, M, V, N, Q, F, W, Y, P, or C; E437 replaced with H, K, R, A, G, I, L, S, T, M, V, N, Q, F, W, Y, P, or C; R438 replaced with D, E, A, G, I, L, S, T, M, V, N, Q, F, W, Y, P, or C; H439 replaced with D, E, A, G, I, L, S, T, M, V, N, Q, F, W, Y, P, or C; A440 replaced with D, E, H, K, R, N, Q, F, W, Y, P, or C; K441 replaced with D, E, A, G, I, L, S, T, M, V, N, Q, F, W, Y, P, or C; E442 replaced with H, K, R, A, G, I, L, S, T, K, V, N, Q, F, W, Y, P, or C; K443 replaced with D, E, A, G, I, L, S, T, M, V, N, Q, F, W, Y, P, or C; I444 replaced with D, E, H, K, R, N, Q, F, W, Y, P, or C; Q445 replaced with D, E, H, K, R, A, G, I, L, S, T, M, V, F, W, Y, P, or C; D446 replaced with H, K, R, A, G, I, L, S, T, M, V, N, Q, F, W, Y, P, or C; L447 replaced with D, E, H, K, R, N, Q, F, W, Y, P, or C; L448 replaced with D, E, H, K, R, N, Q, F, W, Y, P, or C; V449 replaced with D, E, H, K, R, N, Q, F, W, Y, P, or C; D450 replaced with H, K, R, A, G, I, L, S, T, M, V, N, Q, F, W, Y, P, or C; S451 replaced with D, E, H, K, R, N, Q, F, W, Y, P, or C; G452 replaced with D, E, H, K, R, N, Q, F, W, Y, P, or C; K453 replaced with D, E, A, G, I, L, S, T, M, V, N, Q, F, W, Y, P, or C; F454 replaced with D, E, H, K, R, N, Q, A, G, I, L, S, T, M, V, P, or C; I455 replaced with D, E, H, K, R, N, Q, F, W, Y, P, or C; Y456 replaced with D, E, H, K, R, N, Q, A, G, I, L, S, T, M, V, P, or C; L457 replaced with D, E, H, K, R, N, Q, F, W, Y, P, or C; E458 replaced with H, K, R, A, G, I, L, S, T, M, V, N, Q, F, W, Y, P, or C; D459 replaced with H, K, R, A, G, I, L, S, T, M, V, N, Q, F, W, Y, P, or C; G460 replaced with D, E, H, K, R, N, Q, F, W, Y, P, or C; T461 replaced with D, E, H, K, R, N, Q, F, W, Y, P, or C; G462 replaced with D, E, H, K, R, N, Q, F, W, Y, P, or C; S463 replaced with D, E, H, K, R, N, Q, F, W, Y, P, or C; A464 replaced with D, E, H, K, R, N, Q, F, W, Y, P, or C; V465 replaced with D, E, H, K, R, N, Q, F, W, Y, P, or C; S466 replaced with D, E, H, K, R, N, Q, F, W, Y, P, or C; L467 replaced with D, E, H, K, R, N, Q, F, W, Y, P, or C; and/or E468 replaced with K, K, R, A, G, I, L, S, T, M, V, N, Q, F, W, Y, P, or C of SEQ ID NO:1.

Amino acids in the TR4 protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro, or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)). In preferred embodiments, antibodies of the present invention bind regions of TR4 that are essential for TR4 function. In other preferred embodiments, antibodies of the present invention bind regions of TR4 that are essential for TR4 function and inhibit or abolish TR4 function. In other preferred embodiments, antibodies of the present invention bind regions of TR4 that are essential for TR4 function and enhance TR4 function.

Additionally, protein engineering may be employed to improve or alter the characteristics of TR4 polypeptides. Recombinant DNA technology known to those skilled in the art can be used to create novel mutant proteins or muteins including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Antibodies of the present invention may bind such modified TR4 polypeptides.

Non-naturally occurring variants of TR4 may be produced using art-known mutagenesis techniques, which include, but are not limited to oligonucleotide mediated mutagenesis, alanine scanning, PCR mutagenesis, site directed mutagenesis (see e.g., Carter et al., Nucl. Acids Res. 13:4331 (1986); and Zoller et al., Nucl. Acids Res. 10:6487 (1982)), cassette mutagenesis (see e.g., Wells et al., Gene 34:315 (1985)), restriction selection mutagenesis (see e.g., Wells et al., Philos. Trans. R Soc. London SerA 317:415 (1986)).

Thus, the invention also encompasses antibodies that bind TR4 derivatives and analogs that have one or more amino acid residues deleted, added, or substituted to generate TR4 polypeptides that are better suited for expression, scale up, etc., in the host cells chosen. For example, cysteine residues can be deleted or substituted with another amino acid residue in order to eliminate disulfide bridges; N-linked glycosylation sites can be altered or eliminated to achieve, for example, expression of a homogeneous product that is more easily recovered and purified from yeast hosts which are known to hyperglycosylate N-linked sites. To this end, a variety of amino acid substitutions at one or both of the first or third amino acid positions on any one or more of the glycosylation recognition sequences in the TR4 polypeptides and/or an amino acid deletion at the second position of any one or more such recognition sequences will prevent glycosylation of the TR4 at the modified tripeptide sequence (see, e.g., Miyajimo et al., EMBO J 5(6):1193-1197). Additionally, one or more of the amino acid residues of TR4 polypeptides (e.g., arginine and lysine residues) may be deleted or substituted with another residue to eliminate undesired processing by proteases such as, for example, furins or kexins.

The antibodies of the present invention also include antibodies that bind a polypeptide comprising, or alternatively, consisting of the polypeptide encoded by the deposited cDNA (the deposit having ATCC Accession Number 97853) including the leader; a polypeptide comprising, or alternatively, consisting of the mature polypeptide encoded by the deposited the cDNA minus the leader (i.e., the mature protein); a polypeptide comprising, or alternatively, consisting of the polypeptide of SEQ ID NO:1 including the leader; a polypeptide comprising, or alternatively, consisting of the polypeptide of SEQ ID NO:1 minus the amino terminal methionine; a polypeptide comprising, or alternatively, consisting of the polypeptide of SEQ ID NO:1 minus the leader; a polypeptide comprising, or alternatively, consisting of the TR4 extracellular domain; a polypeptide comprising, or alternatively, consisting of the TR4 cysteine rich domain; a polypeptide comprising, or alternatively, consisting of the TR4 transmembrane domain; a polypeptide comprising, or alternatively, consisting of the TR4 intracellular domain; a polypeptide comprising, or alternatively, consisting of the TR4 death domain; a polypeptide comprising, or alternatively, consisting of soluble polypeptides comprising all or part of the extracellular and intracellular domains but lacking the transmembrane domain; as well as polypeptides which are at least 80% identical, more preferably at least 90% or 95% identical, still more preferably at least 96%, 97%, 98% or 99% identical to the polypeptides described above (e.g., the polypeptide encoded by the deposited cDNA clone (the deposit having ATCC Accession Number 97853), the polypeptide of SEQ ID NO:1, and portions of such polypeptides with at least 30 amino acids and more preferably at least 50 amino acids.

By a polypeptide having an amino acid sequence at least, for example, 95% “identical” to a reference amino acid sequence of a TR4 polypeptide is intended that the amino acid sequence of the polypeptide is identical to the reference sequence except that the polypeptide sequence may include up to five amino acid alterations per each 100 amino acids of the reference amino acid of the TR4 polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in SEQ ID NO:1 or to the amino acid sequence encoded by deposited cDNA clones can be determined conventionally using known computer programs such the Bestfit program (Wisconsin Sequence Analysis Package, Version 8 for Unix, Genetics Computer Group, University Research Park, 575 Science Drive, Madison, Wis. 53711. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty-0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of this embodiment.

The present application is also directed to antibodies that bind proteins containing polypeptides at least 90%, 95%, 96%, 97%, 98% or 99% identical to the TR4 polypeptide sequence set forth herein as n¹-m¹, and/or n²-m². In preferred embodiments, the application is directed to antibodies that bind proteins containing polypeptides at least 90%, 95%, 96%, 97%, 98% or 99% identical to polypeptides having the amino acid sequence of the specific TR4 N- and C-terminal deletions recited herein.

In certain preferred embodiments, antibodies of the invention bind TR4 fusion proteins as described above wherein the TR4 portion of the fusion protein are those described as n¹-m¹, and/or n²-m² herein.

TR5

In certain embodiments of the present invention, the antibodies of the present invention bind TR5 polypeptide, or fragments or variants thereof. The following section describes the TR5 polypeptides, fragments and variants that may be bound by the antibodies of the invention in more detail. The TR5 polypeptides, fragments and variants which may be bound by the antibodies of the invention are also described in International Publication Numbers, for example, WO98/30693 and WO00/71150 which are herein incorporated by reference in their entireties. Amino acids 41-299 of SEQ ID NO:2 are identical to the TR5 protein disclosed in WO98/30693 and WO00/71150.

In certain embodiments, the antibodies of the present invention immunospecifically bind TR5 polypeptide. An antibody that immunospecifically binds TR5 may, in some embodiments, bind fragments, variants (including species orthologs of TR5), multimers or modified forms of TR5. For example, an antibody immunospecific for TR5 may bind the TR5 moiety of a fusion protein comprising all or a portion of TR5.

TR5 proteins may be found as monomers or multimers (i.e., dimers, trimers, tetramers, and higher multimers). Accordingly, the present invention relates to antibodies that bind TR5 proteins found as monomers or as part of multimers. In specific embodiments, antibodies of the invention bind TR5 monomers, dimers, trimers or tetramers. In additional embodiments, antibodies of the invention bind at least dimers, at least trimers, or at least tetramers containing one or more TR5 polypeptides.

Antibodies of the invention may bind TR5 homomers or heteromers. As used herein, the term homomer, refers to a multimer containing only TR5 proteins of the invention (including TR5 fragments, variants, and fusion proteins, as described herein). These homomers may contain TR5 proteins having identical or different polypeptide sequences. In a specific embodiment, a homomer of the invention is a multimer containing only TR5 proteins having an identical polypeptide sequence. In another specific embodiment, antibodies of the invention bind TR5 homomers containing TR5 proteins having different polypeptide sequences. In specific embodiments, antibodies of the invention bind a TR5 homodimer (e.g., containing TR5 proteins having identical or different polypeptide sequences) or a homotrimer (e.g., containing TR5 proteins having identical or different polypeptide sequences). In additional embodiments, antibodies of the invention bind at least a homodimer, at least a homotrimer, or at least a homotetramer of TR5.

As used herein, the term heteromer refers to a multimer containing heterologous proteins (i.e., proteins containing polypeptide sequences that do not correspond to a polypeptide sequences encoded by the TR5 gene) in addition to the TR5 proteins of the invention. In a specific embodiment, antibodies of the invention bind a heterodimer, a heterotrimer, or a heterotetramer. In additional embodiments, the antibodies of the invention bind at least a homodimer, at least a homotrimer, or at least a homotetramer containing one or more TR5 polypeptides.

Multimers bound by one or more antibodies of the invention may be the result of hydrophobic, hydrophilic, ionic and/or covalent associations and/or may be indirectly linked, by for example, liposome formation. Thus, in one embodiment, multimers bound by one or more antibodies of the invention, such as, for example, homodimers or homotrimers, are formed when TR5 proteins contact one another in solution. In another embodiment, heteromultimers bound by one or more antibodies of the invention, such as, for example, heterotrimers or heterotetramers, are formed when TR5 proteins contact antibodies to the polypeptides of the invention (including antibodies to the heterologous polypeptide sequence in a fusion protein) in solution. In other embodiments, multimers bound by one or more antibodies of the invention are formed by covalent associations with and/or between the TR5 proteins of the invention. Such covalent associations may involve one or more amino acid residues contained in the polypeptide sequence of the protein (e.g., the polypeptide sequence recited in SEQ ID NO:2 or the polypeptide encoded by the deposited cDNA clone of ATCC Deposit 97798). In one instance, the covalent associations are cross-linking between cysteine residues located within the polypeptide sequences of the proteins which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a TR5 fusion protein. In one example, covalent associations are between the heterologous sequence contained in a fusion protein (see, e.g., U.S. Pat. No. 5,478,925). In a specific example, the covalent associations are between the heterologous sequence contained in a TR5-Fc fusion protein (as described herein). In another specific example, covalent associations of fusion proteins are between heterologous polypeptide sequences from another TNF family ligand/receptor member that is capable of forming covalently associated multimers, such as for example, osteoprotegerin (see, e.g., International Publication No. WO 98/49305, the contents of which are herein incorporated by reference in its entirety).

The multimers that may be bound by one or more antibodies of the invention may be generated using chemical techniques known in the art. For example, proteins desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers that may be bound by one or more antibodies of the invention may be generated using techniques known in the art to form one or more inter-molecule cross-links between the cysteine residues located within the polypeptide sequence of the proteins desired to be contained in the multimer (see, e.g., U.S. Pat. No. 5,478,925, which is herein incorporated by reference in its entirety). Further, proteins that may be bound by one or more antibodies of the invention may be routinely modified by the addition of cysteine or biotin to the C terminus or N-terminus of the polypeptide sequence of the protein and techniques known in the art may be applied to generate multimers containing one or more of these modified proteins (see, e.g., U.S. Pat. No. 5,478,925, which is herein incorporated by reference in its entirety). Additionally, techniques known in the art may be applied to generate liposomes containing the protein components desired to be contained in the multimer that may be bound by one or more antibodies of the invention (see, e.g., U.S. Pat. No. 5,478,925, which is herein incorporated by reference in its entirety).

Alternatively, multimers that may be bound by one or more antibodies of the invention may be generated using genetic engineering techniques known in the art. In one embodiment, proteins contained in multimers that may be bound by one or more antibodies of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer that may be bound by one or more antibodies of the invention are generated by ligating a polynucleotide sequence encoding a TR5 polypeptide to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant TR5 polypeptides which contain a transmembrane domain and which can be incorporated by membrane reconstitution techniques into liposomes (see, e.g., U.S. Pat. No. 5,478,925, which is herein incorporated by reference in its entirety). In another embodiment, two or more TR5 polypeptides of the invention are joined through synthetic linkers (e.g., peptide, carbohydrate or soluble polymer linkers). Examples include those peptide linkers described in U.S. Pat. No. 5,073,627 (hereby incorporated by reference). Proteins comprising multiple TR5 polypeptides separated by peptide linkers may be produced using conventional recombinant DNA technology. In specific embodiments, antibodies of the invention bind proteins comprising multiple TR5 polypeptides separated by peptide linkers.

Another method for preparing multimer TR5 polypeptides of the invention involves use of TR5 polypeptides fused to a leucine zipper or isoleucine polypeptide sequence. Leucine zipper domains and isoleucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric TR5 proteins are those described in PCT application WO 94/10308, hereby incorporated by reference. Recombinant fusion proteins comprising a soluble TR5 polypeptide fused to a peptide that dimerizes or trimerizes in solution are expressed in suitable host cells, and the resulting soluble multimeric TR5 is recovered from the culture supernatant using techniques known in the art. In specific embodiments, antibodies of the invention bind TR5-leucine zipper fusion protein monomers and/or TR5-leucine zipper fusion protein multimers.

Certain members of the TNF family of proteins are believed to exist in trimeric form (Beutler and Huffel, Science 264:667, 1994; Banner et al., Cell 73:431, 1993). Thus, trimeric TR5 may offer the advantage of enhanced biological activity. Preferred leucine zipper moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. In specific embodiments, antibodies of the invention bind TR5-leucine zipper fusion protein trimers.

Other peptides derived from naturally occurring trimeric proteins may be employed in preparing trimeric TR5. In specific embodiments, antibodies of the invention bind TR5-fusion protein monomers and/or TR5 fusion protein trimers.

The TR5 polypeptides are preferably provided in an isolated form, and preferably are substantially purified. By “isolated polypeptide” is intended a polypeptide removed from its native environment. Thus, a polypeptide produced and/or contained within a recombinant host cell is considered isolated for purposes of the present invention. Also, intended as an “isolated polypeptide” are polypeptides that have been purified, partially or substantially, from a recombinant host cell. For example, a recombinantly produced version of the TR5 polypeptide is substantially purified by the one-step method described in Smith and Johnson, Gene 67:31-40 (1988).

Antibodies of the present invention may bind TR5 polypeptides or polypeptide fragments including polypeptides comprising or alternatively, consisting of, an amino acid sequence contained in SEQ ID NO:2, encoded by the cDNA contained in ATCC deposit Number 97798, or encoded by nucleic acids which hybridize (e.g., under stringent hybridization conditions) to the cDNA contained in ATCC deposit Number 97798 or to SEQ ID NO:1 or the complementary strand thereto. Protein fragments may be “free-standing,” or comprised within a larger polypeptide of which the fragment forms a part or region, most preferably as a single continuous region. Antibodies of the present invention may bind polypeptide fragments, including, for example, fragments that comprise or alternatively, consist of from about amino acid residues: 1 to 40, 41 to 66, 67 to 90, 91 to 140, 191 to 240, 241 to 280, and/or 281 to 299, of SEQ ID NO:2. Moreover, polypeptide fragments bound by the antibodies of the invention can be at least 10, 20, 30, 40, 50, 60, 70, 80, 90, 100, 110, 120, 130, 140, 150, 175 or 200 amino acids in length.

In specific embodiments, antibodies of the present invention bind polypeptide fragments comprising, or alternatively consisting of, amino acid residues: 41-299, 67-299, 67-280, 93-193, and/or 281-299, of TR5 (SEQ ID NO:2). Polynucleotides encoding these polypeptides are also encompassed by the invention.

In additional embodiments, antibodies of the present invention bind TR5 polypeptide fragments comprising, or alternatively consisting, of one or more TR5 domains. Preferably, antibodies of the present invention bind TR5 polypeptides or polypeptide fragments selected from the group consisting of: (a) a polypeptide comprising or alternatively, consisting of, the TR5 transmembrane domain (predicted to constitute amino acid residues from about 281 to about 299 of SEQ ID NO:2); (b) a polypeptide comprising or alternatively, consisting of, the TR5 receptor extracellular domain (predicted to constitute amino acid residues from about 67 to about 280 of SEQ ID NO:2); (c) a polypeptide comprising or alternatively, consisting of, both TR5 cysteine rich domains (both of which may be found in the protein fragment consisting of amino acid residues from about 93 to about 193 in SEQ ID NO:2); (d) a polypeptide comprising or alternatively, consisting of, the TR5 cysteine rich domain consisting of amino acid residues from about 93-150 in SEQ ID NO:2); (e) a polypeptide comprising or alternatively, consisting of, the TR5 cysteine rich domain consisting of amino acid residues from about 151 to about 193 in SEQ ID NO:2); (f) a polypeptide comprising or alternatively, consisting of, fragment of the predicted mature TR5 polypeptide, wherein the fragment has a TR5 functional activity (e.g., antigenic activity or biological activity); or (g) any combination of polypeptides (a)-(g). As discussed above, it is believed that one or both of the extracellular cysteine rich motifs of TR5 is important for interactions between TR5 and its ligands (e.g., TRAIL). Accordingly, in highly preferred embodiments, antibodies of the present invention bind TR5 polypeptide fragments comprising, or alternatively consisting of, amino acid residues 93 to 150 and/or 151-193 of SEQ ID NO:2. In another highly preferred embodiment, antibodies of the present invention bind TR5 polypeptides comprising, or alternatively consisting of, both of the extracellular cysteine rich motifs (amino acid residues 93 to 193 SEQ ID NO:2.) In another preferred embodiment, antibodies of the present invention bind TR5 polypeptides comprising, or alternatively consisting of the extracellular soluble domain of TR5 (amino acid residues 67-280 of SEQ ID NO:2.)

Antibodies of the invention may also bind fragments comprising, or alternatively, consisting of structural or functional attributes of TR5. Such fragments include amino acid residues that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, surface forming regions, and high antigenic index regions (i.e., containing four or more contiguous amino acids having an antigenic index of greater than or equal to 1.5, as identified using the default parameters of the Jameson-Wolf program) of complete (i.e., full-length) TR5. Certain preferred regions are those set out in Table 4 and include, but are not limited to, regions of the aforementioned types identified by analysis of the amino acid sequence depicted in (SEQ ID NO:2), such preferred regions include; Garnier-Robson predicted alpha-regions, beta-regions, turn-regions, and coil-regions; Chou-Fasman predicted alpha-regions, beta-regions, and turn-regions; Kyte-Doolittle predicted hydrophilic regions; Eisenberg alpha and beta amphipathic regions; Emini surface-forming regions; and Jameson-Wolf high antigenic index regions, as predicted using the default parameters of these computer programs.

The data representing the structural or functional attributes of TR5 set forth in Table 4, as described above, was generated using the various modules and algorithms of the DNA*STAR set on default parameters. Column I represents the results of a Garnier-Robson analysis of alpha helical regions; Column II represents the results of a Chou-Fasman analysis of alpha helical regions; Column III represents the results of a Garnier Robson analysis of beta sheet regions; Column IV represents the results of a Chou-Fasman analysis of beta sheet regions; Column V represents the results of a Garnier Robson analysis of turn regions; Column VI represents the results of a Chou-Fasman analysis of turn regions; Column VII represents the results of a Garnier Robson analysis of coil regions; Column VIII represents a Kyte-Doolittle hydrophilicity plot; Column IX represents a Hopp-Woods hydrophobicity plot; Column X represents the results of an Eisenberg analysis of alpha amphipathic regions; Column XI represents the results of an Eisenberg analysis of beta amphipathic regions; Column XII represents the results of a Karplus-Schultz analysis of flexible regions; Column XIII represents the Jameson-Wolf antigenic index score; and Column XIV represents the Emini surface probability plot.

In a preferred embodiment, the data presented in columns VIII, IX, XIII, and XIV of Table 4 can be used to determine regions of TR5 which exhibit a high degree of potential for antigenicity. Regions of high antigenicity are determined from the data presented in columns VIII, IX, XIII, and/or XIV by choosing values which represent regions of the polypeptide which are likely to be exposed on the surface of the polypeptide in an environment in which antigen recognition may occur in the process of initiation of an immune response.

The above-mentioned preferred regions set out in Table 4 include, but are not limited to, regions of the aforementioned types identified by analysis of the amino acid sequence set out in SEQ ID NO:2. As set out in Table 4, such preferred regions include Garnier-Robson alpha-regions, beta-regions, turn-regions, and coil-regions, Chou-Fasman alpha-regions, beta-regions, and turn-regions, Kyte-Doolittle hydrophilic regions, Eisenberg alpha- and beta-amphipathic regions, Karplus-Schulz flexible regions, Jameson-Wolf regions of high antigenic index and Emini surface-forming regions. Preferably, antibodies of the present invention bind TR5 polypeptides or TR5 polypeptide fragments and variants comprising regions of TR5 that combine several structural features, such as several (e.g., 1, 2, 3, or 4) of the same or different region features set out above and in Table 4.

TABLE 4

| Res | Position | I | II | III | IV | V | VI | VII | VIII | IX | X | XI | XII | XIII | XIV |
|---|---|---|---|---|---|---|---|---|---|---|---|---|---|---|---|
| Met | 1 | . | . | . | . | . | . | C | 0.19 | 0.37 | * | . | . | 0.10 | 0.47 |
| Gln | 2 | . | . | . | . | . | . | C | 0.58 | −0.06 | . | * | . | 0.70 | 0.74 |
| Gly | 3 | . | A | . | . | . | . | C | 1.08 | −0.49 | * | * | . | 0.50 | 1.00 |
| Val | 4 | . | A | . | . | . | . | C | 0.77 | −0.91 | * | * | . | 0.95 | 1.98 |
| Lys | 5 | . | A | . | . | . | . | C | 0.34 | −0.74 | * | * | F | 0.95 | 0.99 |
| Glu | 6 | . | A | B | . | . | . | . | 0.73 | −0.46 | * | * | F | 0.45 | 0.82 |
| Arg | 7 | . | A | B | . | . | . | . | −0.08 | −0.46 | * | * | F | 0.60 | 1.72 |
| Phe | 8 | . | . | B | . | . | . | . | −0.08 | −0.41 | * | * | . | 0.50 | 0.71 |
| Leu | 9 | . | . | B | . | . | T | . | 0.78 | 0.01 | * | * | . | 0.10 | 0.40 |
| Pro | 10 | . | . | . | . | . | T | C | 0.43 | 0.41 | * | * | . | 0.00 | 0.33 |
| Leu | 11 | . | . | . | . | T | T | . | 0.09 | 0.80 | * | . | F | 0.69 | 0.51 |
| Gly | 12 | . | . | . | . | T | T | . | −0.02 | 0.44 | * | * | F | 1.03 | 0.62 |
| Asn | 13 | . | . | . | . | T | T | . | 0.79 | −0.24 | * | . | F | 2.27 | 0.67 |
| Ser | 14 | . | . | . | . | . | T | C | 1.01 | −0.67 | * | * | F | 2.86 | 1.58 |
| Gly | 15 | . | . | . | . | T | T | . | 1.01 | −0.86 | * | . | F | 3.40 | 1.62 |
| Asp | 16 | . | . | . | . | T | T | . | 1.93 | −0.86 | * | . | F | 3.06 | 1.55 |
| Arg | 17 | . | . | . | . | . | . | C | 2.07 | −1.26 | * | . | F | 2.32 | 2.27 |
| Ala | 18 | . | . | . | . | . | . | C | 1.86 | −1.21 | * | . | F | 2.32 | 3.55 |
| Pro | 19 | . | . | . | . | . | . | C | 2.16 | −1.21 | * | . | F | 2.32 | 3.28 |
| Arg | 20 | . | . | . | . | . | . | C | 2.16 | −1.21 | * | * | F | 2.32 | 2.80 |
| Pro | 21 | . | . | . | . | . | T | C | 2.27 | −0.79 | * | * | F | 2.86 | 2.74 |
| Pro | 22 | . | . | . | . | T | T | . | 1.81 | −1.29 | * | * | F | 3.40 | 3.47 |
| Asp | 23 | . | . | . | . | T | T | . | 2.51 | −1.29 | * | * | F | 3.06 | 1.76 |
| Gly | 24 | . | . | . | . | T | T | . | 1.87 | −1.29 | . | * | F | 2.72 | 2.22 |
| Arg | 25 | . | . | . | B | T | . | . | 1.87 | −1.07 | . | * | F | 1.98 | 1.07 |
| Gly | 26 | . | . | . | B | T | . | . | 1.87 | −1.50 | . | * | F | 1.64 | 1.25 |
| Arg | 27 | . | . | . | B | T | . | . | 2.19 | −1.07 | . | * | F | 1.30 | 1.96 |
| Val | 28 | . | . | B | B | . | . | . | 1.88 | −1.50 | . | * | F | 1.24 | 1.96 |
| Arg | 29 | . | . | B | B | . | . | . | 2.22 | −1.01 | . | * | F | 1.58 | 2.85 |
| Pro | 30 | . | . | B | . | . | . | . | 2.11 | −1.04 | . | * | F | 2.12 | 2.52 |
| Arg | 31 | . | . | . | . | T | . | . | 2.11 | −1.04 | * | * | F | 2.86 | 5.68 |
| Thr | 32 | . | . | . | . | T | T | . | 1.14 | −1.26 | * | * | F | 3.40 | 2.87 |
| Gln | 33 | . | . | . | . | T | T | . | 1.66 | −0.61 | . | * | F | 3.06 | 1.38 |
| Asp | 34 | . | . | . | . | T | T | . | 1.54 | −0.61 | . | * | F | 2.57 | 0.70 |
| Gly | 35 | . | . | . | . | T | T | . | 1.72 | −0.21 | * | * | F | 1.93 | 0.77 |
| Val | 36 | . | . | . | . | . | . | C | 1.30 | −0.20 | * | * | F | 1.19 | 0.61 |
| Gly | 37 | . | . | . | . | . | . | C | 1.01 | −0.11 | . | . | F | 0.85 | 0.53 |
| Asn | 38 | . | A | . | . | . | . | C | 0.42 | 0.50 | . | * | . | −0.40 | 0.53 |
| His | 39 | . | A | . | . | . | . | C | 0.53 | 0.57 | * | * | . | −0.40 | 0.72 |
| Thr | 40 | . | A | . | . | . | . | C | −0.01 | −0.07 | * | * | . | 0.65 | 1.42 |
| Met | 41 | . | A | B | . | . | . | . | 0.63 | 0.19 | * | * | . | −0.30 | 0.62 |
| Ala | 42 | . | A | B | . | . | . | . | 1.02 | 0.21 | * | . | . | 0.00 | 0.70 |
| Arg | 43 | . | A | B | . | . | . | . | 0.71 | −0.29 | * | * | . | 0.90 | 0.97 |
| Ile | 44 | . | . | B | . | . | . | . | −0.07 | −0.29 | * | * | . | 1.55 | 1.42 |
| Pro | 45 | . | . | . | . | . | . | C | 0.29 | −0.21 | * | * | F | 2.20 | 1.16 |
| Lys | 46 | . | . | . | . | T | . | . | 0.19 | −0.71 | * | * | F | 3.00 | 1.18 |
| Thr | 47 | . | . | . | B | . | . | C | −0.08 | 0.07 | * | * | F | 1.40 | 1.46 |
| Leu | 48 | . | . | . | B | . | . | C | −1.04 | 0.03 | * | * | F | 0.95 | 0.70 |
| Lys | 49 | . | . | B | B | . | . | . | −1.01 | 0.24 | * | * | . | 0.30 | 0.26 |
| Phe | 50 | . | . | B | B | . | . | . | −1.69 | 0.89 | * | . | . | −0.30 | 0.13 |
| Val | 51 | . | . | B | B | . | . | . | −2.59 | 1.09 | * | * | . | −0.60 | 0.11 |
| Val | 52 | . | . | B | B | . | . | . | −2.87 | 1.04 | . | * | . | −0.60 | 0.04 |
| Val | 53 | . | . | B | B | . | . | . | −2.91 | 1.54 | * | * | . | −0.60 | 0.05 |
| Ile | 54 | . | . | B | B | . | . | . | −3.77 | 1.40 | * | * | . | −0.60 | 0.05 |
| Val | 55 | . | . | B | B | . | . | . | −3.88 | 1.44 | . | . | . | −0.60 | 0.05 |
| Ala | 56 | . | . | B | B | . | . | . | −3.23 | 1.49 | . | . | . | −0.60 | 0.06 |
| Val | 57 | . | . | B | B | . | . | . | −3.23 | 1.27 | . | . | . | −0.60 | 0.13 |
| Leu | 58 | . | . | B | B | . | . | . | −3.19 | 1.23 | . | . | . | −0.60 | 0.13 |
| Leu | 59 | . | . | B | B | . | . | . | −2.89 | 1.27 | * | . | . | −0.60 | 0.11 |
| Pro | 60 | . | . | B | B | . | . | . | −2.28 | 1.27 | . | . | . | −0.60 | 0.15 |
| Val | 61 | . | . | B | B | . | . | . | −1.99 | 1.39 | . | . | . | −0.60 | 0.28 |
| Leu | 62 | . | . | B | B | . | . | . | −1.72 | 1.09 | . | . | . | −0.60 | 0.46 |
| Ala | 63 | . | . | B | B | . | . | . | −1.22 | 0.90 | . | . | . | −0.60 | 0.30 |
| Tyr | 64 | . | . | B | B | . | . | . | −0.72 | 0.96 | . | . | . | −0.60 | 0.58 |
| Ser | 65 | . | . | . | B | . | . | C | −1.10 | 0.80 | * | * | . | −0.25 | 1.02 |
| Ala | 66 | . | . | . | B | . | . | C | −0.13 | 0.61 | * | . | . | −0.25 | 1.02 |
| Thr | 67 | . | . | . | B | . | . | C | 0.68 | 0.11 | . | * | F | 0.20 | 1.28 |
| Thr | 68 | . | A | . | B | . | . | C | 1.27 | −0.24 | . | . | F | 0.80 | 1.65 |
| Ala | 69 | . | A | . | B | . | . | C | 1.51 | −0.63 | . | . | F | 1.10 | 2.83 |
| Arg | 70 | . | A | . | B | . | . | C | 0.96 | −1.13 | . | . | F | 1.10 | 3.40 |
| Gln | 71 | . | A | . | B | . | . | C | 1.33 | −0.97 | . | . | F | 1.10 | 1.75 |
| Glu | 72 | . | A | . | B | . | . | C | 1.64 | −1.03 | . | . | F | 1.10 | 2.68 |
| Glu | 73 | . | A | . | . | . | . | C | 1.96 | −1.13 | . | . | F | 1.10 | 2.37 |
| Val | 74 | . | A | . | . | . | . | C | 2.23 | −0.73 | * | . | F | 1.10 | 2.37 |
| Pro | 75 | . | A | . | . | T | . | . | 1.27 | −0.64 | * | . | F | 1.30 | 1.97 |
| Gln | 76 | . | . | . | B | T | . | . | 0.68 | 0.00 | . | . | F | 0.85 | 0.85 |
| Gln | 77 | . | . | . | B | . | . | C | 0.47 | 0.50 | . | . | F | −0.10 | 1.15 |
| Thr | 78 | . | . | . | B | . | . | C | 0.47 | 0.29 | . | . | F | 0.20 | 1.15 |
| Val | 79 | . | . | . | B | . | . | C | 1.32 | 0.26 | . | . | F | 0.20 | 1.15 |
| Ala | 80 | . | . | . | B | . | . | C | 1.53 | 0.26 | * | . | F | 0.20 | 1.15 |
| Pro | 81 | . | . | . | B | . | . | C | 1.64 | 0.26 | . | . | F | 0.20 | 1.38 |
| Gln | 82 | . | . | . | . | T | . | . | 1.61 | −0.23 | * | . | F | 1.54 | 3.64 |
| Gln | 83 | . | . | . | . | . | . | C | 1.62 | −0.37 | . | * | F | 1.68 | 4.91 |
| Gln | 84 | . | . | . | . | T | T | . | 1.78 | −0.49 | * | * | F | 2.42 | 4.25 |
| Arg | 85 | . | . | . | . | T | T | . | 2.41 | −0.13 | * | * | F | 2.76 | 2.13 |
| His | 86 | . | . | . | . | T | T | . | 2.28 | −0.53 | * | * | F | 3.40 | 2.46 |
| Ser | 87 | . | . | . | . | . | T | C | 2.28 | −0.50 | * | * | . | 2.71 | 1.40 |
| Phe | 88 | . | A | . | . | T | . | . | 2.28 | −0.90 | * | * | F | 2.32 | 1.24 |
| Lys | 89 | . | A | . | . | T | . | . | 1.61 | −0.90 | * | * | F | 1.98 | 1.58 |
| Gly | 90 | . | A | . | . | T | . | . | 1.29 | −0.83 | * | * | F | 1.80 | 0.63 |
| Glu | 91 | . | A | . | . | T | . | . | 0.73 | −0.79 | . | * | F | 1.92 | 1.13 |
| Glu | 92 | . | A | . | . | . | . | C | 0.69 | −1.07 | . | * | F | 1.88 | 0.57 |
| Cys | 93 | . | . | . | . | . | T | C | 1.09 | −0.64 | . | * | F | 2.59 | 0.57 |
| Pro | 94 | . | . | . | . | T | T | . | 1.01 | −0.69 | * | * | F | 3.10 | 0.44 |
| Ala | 95 | . | . | . | . | T | T | . | 1.47 | −0.19 | * | . | F | 2.49 | 0.35 |
| Gly | 96 | . | . | . | . | . | T | C | 1.17 | −0.19 | . | * | F | 2.13 | 1.27 |
| Ser | 97 | . | . | . | . | . | . | C | 1.17 | −0.37 | . | * | F | 1.62 | 1.10 |
| His | 98 | . | . | . | . | . | . | C | 1.80 | −0.80 | . | * | F | 1.95 | 1.88 |
| Arg | 99 | . | . | . | . | T | . | . | 1.70 | −0.80 | . | * | F | 2.18 | 2.59 |
| Ser | 100 | . | . | . | . | T | . | . | 1.94 | −0.74 | . | * | F | 2.52 | 2.78 |
| Glu | 101 | . | . | . | . | T | . | . | 1.70 | −0.70 | . | * | F | 2.86 | 2.03 |
| His | 102 | . | . | . | . | T | T | . | 1.33 | −0.70 | . | * | F | 3.40 | 1.04 |
| Thr | 103 | . | . | . | . | T | T | . | 1.37 | −0.13 | * | * | F | 2.61 | 0.42 |
| Gly | 104 | . | . | . | . | T | T | . | 1.04 | −0.11 | * | * | . | 2.12 | 0.39 |
| Ala | 105 | . | . | . | . | T | T | . | 0.68 | 0.31 | . | . | . | 1.18 | 0.44 |
| Cys | 106 | . | . | . | . | T | . | . | 0.37 | 0.39 | . | . | . | 0.64 | 0.16 |
| Asn | 107 | . | . | . | . | . | T | C | 0.40 | 0.39 | . | . | . | 0.30 | 0.24 |
| Pro | 108 | . | . | . | . | T | T | . | 0.37 | −0.04 | * | . | F | 1.53 | 0.41 |
| Cys | 109 | . | . | . | . | T | T | . | −0.14 | −0.11 | * | . | F | 1.81 | 0.76 |
| Thr | 110 | . | . | . | . | T | T | . | 0.44 | −0.04 | * | . | F | 2.09 | 0.35 |
| Glu | 111 | . | . | B | . | . | . | . | 0.87 | −0.44 | * | . | F | 1.77 | 0.38 |
| Gly | 112 | . | . | . | . | T | T | . | 0.56 | −0.11 | * | . | F | 2.80 | 1.10 |
| Val | 113 | . | . | B | . | . | T | . | 0.77 | −0.20 | . | . | F | 2.12 | 1.10 |
| Asp | 114 | . | . | B | . | . | T | . | 0.84 | −0.29 | . | . | . | 1.69 | 1.02 |
| Tyr | 115 | . | . | . | . | . | T | C | 0.86 | 0.21 | . | . | . | 1.01 | 1.04 |
| Thr | 116 | . | . | . | . | . | . | C | 0.86 | 0.17 | . | . | F | 0.68 | 1.88 |
| Asn | 117 | . | . | . | . | . | . | C | 1.20 | −0.07 | . | . | F | 1.34 | 1.81 |
| Ala | 118 | . | . | . | . | . | T | C | 2.06 | 0.33 | . | . | F | 1.28 | 1.86 |
| Ser | 119 | . | . | . | . | T | T | . | 1.84 | −0.43 | . | . | F | 2.42 | 2.24 |
| Asn | 120 | . | . | . | . | T | T | . | 1.79 | −0.49 | . | . | F | 2.76 | 2.15 |
| Asn | 121 | . | . | . | . | T | T | . | 1.43 | −0.50 | * | . | F | 3.40 | 2.85 |
| Glu | 122 | . | . | . | . | . | T | C | 0.73 | −0.43 | * | . | F | 2.56 | 1.14 |
| Pro | 123 | . | . | . | . | T | T | . | 1.11 | −0.03 | . | . | F | 2.27 | 0.61 |
| Ser | 124 | . | . | . | . | T | T | . | 0.74 | 0.00 | . | . | F | 1.93 | 0.59 |
| Cys | 125 | . | . | . | . | T | T | . | 0.43 | 0.17 | * | . | . | 0.84 | 0.18 |
| Phe | 126 | . | . | B | . | . | T | . | −0.42 | 0.66 | . | . | . | −0.20 | 0.17 |
| Pro | 127 | . | . | . | . | T | T | . | −1.09 | 0.87 | * | . | . | 0.20 | 0.09 |
| Cys | 128 | . | . | . | . | T | T | . | −0.83 | 1.06 | * | . | . | 0.20 | 0.09 |
| Thr | 129 | . | . | . | . | T | T | . | −0.83 | 0.49 | . | . | . | 0.54 | 0.22 |
| Val | 130 | . | . | . | . | T | . | . | −0.17 | 0.09 | * | . | . | 0.98 | 0.19 |
| Cys | 131 | . | . | . | . | T | T | . | 0.53 | −0.34 | * | * | . | 2.12 | 0.59 |
| Lys | 132 | . | . | . | . | T | T | . | 0.79 | −0.51 | * | . | F | 2.91 | 0.71 |
| Ser | 133 | . | . | . | . | T | T | . | 1.42 | −1.00 | . | * | F | 3.40 | 1.90 |
| Asp | 134 | . | . | . | . | T | T | . | 1.78 | −1.14 | * | * | F | 3.06 | 4.84 |
| Gln | 135 | . | . | . | . | T | . | . | 2.33 | −1.71 | * | * | F | 2.83 | 4.84 |
| Lys | 136 | . | . | . | . | T | . | . | 2.70 | −1.33 | . | * | F | 2.80 | 4.84 |
| His | 137 | . | . | . | . | T | T | . | 1.99 | −1.33 | . | * | F | 2.97 | 3.88 |
| Lys | 138 | . | . | . | . | T | T | . | 1.98 | −0.76 | . | * | F | 2.94 | 1.20 |
| Ser | 139 | . | . | . | . | T | T | . | 1.38 | −0.67 | . | * | F | 3.10 | 0.87 |
| Ser | 140 | . | . | . | . | T | T | . | 1.07 | −0.06 | * | * | F | 2.49 | 0.63 |
| Cys | 141 | . | . | . | B | T | . | . | 1.13 | −0.07 | * | * | F | 1.78 | 0.45 |
| Thr | 142 | . | . | . | B | T | . | . | 1.17 | −0.07 | * | . | . | 1.32 | 0.66 |
| Met | 143 | . | . | . | B | T | . | . | 0.81 | −0.46 | * | * | . | 1.01 | 0.83 |
| Thr | 144 | . | . | . | . | T | T | . | 0.26 | −0.36 | . | . | F | 1.40 | 2.23 |
| Arg | 145 | . | . | . | . | T | T | . | −0.11 | −0.29 | . | . | F | 1.40 | 1.15 |
| Asp | 146 | . | . | . | . | T | T | . | 0.56 | −0.20 | * | . | F | 1.25 | 0.62 |
| Thr | 147 | . | . | . | . | T | T | . | 0.20 | −0.41 | * | . | . | 1.10 | 0.75 |
| Val | 148 | . | . | . | B | T | . | . | 0.84 | −0.33 | * | . | . | 1.04 | 0.20 |
| Cys | 149 | . | . | . | B | T | . | . | 1.16 | −0.33 | * | . | . | 1.38 | 0.24 |
| Gln | 150 | . | . | . | B | T | . | . | 0.70 | −0.33 | * | . | . | 1.72 | 0.29 |
| Cys | 151 | . | . | . | . | T | T | . | 0.39 | −0.39 | . | . | . | 2.46 | 0.39 |
| Lys | 152 | . | . | . | . | T | T | . | 0.00 | −0.54 | * | * | F | 3.40 | 1.05 |
| Glu | 153 | . | . | . | . | T | T | . | 0.97 | −0.33 | * | * | F | 2.61 | 0.53 |
| Gly | 154 | . | . | . | . | T | T | . | 1.63 | −0.73 | . | * | F | 3.06 | 1.92 |
| Thr | 155 | . | . | . | . | T | . | . | 1.63 | −0.90 | * | * | F | 2.86 | 1.55 |
| Phe | 156 | . | . | . | . | . | . | C | 2.30 | −0.90 | . | * | F | 2.66 | 1.55 |
| Arg | 157 | . | . | . | . | T | . | . | 1.96 | −0.50 | . | * | F | 2.86 | 2.51 |
| Asn | 158 | . | . | . | . | T | T | . | 1.74 | −0.54 | * | * | F | 3.40 | 2.33 |
| Glu | 159 | . | . | . | . | T | T | . | 2.09 | −0.60 | . | * | F | 3.06 | 4.17 |
| Asn | 160 | . | . | . | . | . | T | C | 1.80 | −1.39 | . | * | F | 2.52 | 3.68 |
| Ser | 161 | . | . | . | . | . | T | C | 1.83 | −0.77 | . | * | F | 2.18 | 2.27 |
| Pro | 162 | . | . | . | . | T | . | . | 1.83 | −0.60 | * | * | F | 1.69 | 0.70 |
| Glu | 163 | . | . | . | . | T | . | . | 1.88 | −0.60 | * | . | F | 1.66 | 0.85 |
| Met | 164 | . | . | . | . | T | . | . | 1.21 | −1.00 | * | . | . | 1.97 | 1.28 |
| Cys | 165 | . | . | . | . | T | T | . | 0.91 | −0.81 | * | . | . | 2.33 | 0.44 |
| Arg | 166 | . | . | . | . | T | T | . | 1.32 | −0.86 | * | * | . | 2.64 | 0.34 |
| Lys | 167 | . | . | . | . | T | T | . | 0.87 | −0.86 | * | * | F | 3.10 | 0.68 |
| Cys | 168 | . | . | . | . | T | T | . | 0.66 | −0.90 | * | * | F | 2.79 | 0.68 |
| Ser | 169 | . | . | . | . | T | . | . | 0.96 | −1.04 | * | * | F | 2.59 | 0.53 |
| Arg | 170 | . | . | . | . | T | . | . | 1.28 | −0.66 | * | * | F | 2.59 | 0.36 |
| Cys | 171 | . | . | . | . | . | T | C | 1.17 | −0.23 | * | * | F | 2.29 | 0.66 |
| Pro | 172 | . | . | . | . | T | T | . | 0.27 | −0.80 | * | . | F | 2.79 | 0.85 |
| Ser | 173 | . | . | . | . | T | T | . | 0.93 | −0.54 | * | * | F | 3.10 | 0.32 |
| Gly | 174 | . | . | . | . | T | T | . | 0.38 | −0.14 | * | * | F | 2.64 | 1.05 |
| Glu | 175 | . | . | . | B | T | . | . | −0.03 | −0.07 | * | * | F | 1.78 | 0.50 |
| Val | 176 | . | . | B | B | . | . | . | 0.63 | −0.11 | . | * | F | 1.07 | 0.50 |
| Gln | 177 | . | . | B | B | . | . | . | 0.18 | −0.10 | . | * | . | 0.61 | 0.82 |
| Val | 178 | . | . | B | . | . | T | . | 0.17 | 0.04 | . | * | . | 0.10 | 0.25 |
| Ser | 179 | . | . | . | . | T | T | . | 0.21 | 0.53 | . | * | . | 0.20 | 0.49 |
| Asn | 180 | . | . | . | . | T | T | . | −0.08 | 0.27 | . | * | F | 0.65 | 0.38 |
| Cys | 181 | . | . | . | . | T | T | . | 0.78 | 0.79 | . | * | F | 0.63 | 0.54 |
| Thr | 182 | . | . | . | . | T | . | . | 0.78 | 0.14 | . | . | F | 1.01 | 0.67 |
| Ser | 183 | . | . | . | . | T | . | . | 0.74 | −0.24 | . | . | F | 1.89 | 0.70 |
| Trp | 184 | . | . | . | . | T | T | . | 1.04 | 0.04 | . | . | F | 1.77 | 0.91 |
| Asp | 185 | . | . | . | . | T | T | . | 0.38 | −0.13 | . | . | F | 2.80 | 1.09 |
| Asp | 186 | . | . | . | . | T | T | . | 0.19 | −0.04 | * | . | F | 2.37 | 0.44 |
| Ile | 187 | . | . | . | . | . | T | C | 0.50 | 0.21 | * | . | . | 1.14 | 0.31 |
| Gln | 188 | . | A | B | . | . | . | . | 0.80 | −0.70 | * | . | . | 1.16 | 0.32 |
| Cys | 189 | . | A | B | . | . | . | . | 0.39 | −0.70 | * | . | . | 0.88 | 0.33 |
| Val | 190 | . | A | B | . | . | . | . | 0.04 | 0.09 | * | * | . | −0.30 | 0.41 |
| Glu | 191 | . | A | . | . | . | . | C | −0.54 | −0.17 | * | * | . | 0.50 | 0.23 |
| Glu | 192 | . | A | . | . | T | . | . | 0.34 | −0.07 | * | * | . | 0.70 | 0.44 |
| Phe | 193 | . | A | . | . | T | . | . | −0.24 | −0.24 | * | * | . | 0.88 | 0.96 |
| Gly | 194 | . | . | . | . | T | T | . | 0.11 | −0.39 | . | * | . | 1.46 | 0.56 |
| Ala | 195 | . | . | . | . | . | T | C | 0.11 | 0.10 | . | * | . | 0.84 | 0.47 |
| Asn | 196 | . | . | . | . | . | T | C | 0.11 | 0.74 | . | * | . | 0.72 | 0.40 |
| Ala | 197 | . | . | . | . | . | T | C | −0.20 | −0.04 | . | * | . | 1.80 | 0.70 |
| Thr | 198 | . | A | . | . | . | . | C | 0.29 | 0.01 | . | * | . | 0.62 | 1.00 |
| Val | 199 | . | A | . | . | . | . | C | 0.04 | −0.06 | . | * | F | 1.19 | 0.96 |
| Glu | 200 | . | A | . | . | . | . | C | 0.04 | 0.04 | . | * | F | 0.41 | 0.96 |
| Thr | 201 | . | A | . | . | . | . | C | 0.04 | 0.04 | . | * | F | 0.23 | 0.67 |
| Pro | 202 | . | A | . | . | . | . | C | 0.63 | −0.44 | . | * | F | 0.80 | 1.57 |
| Ala | 203 | A | A | . | . | . | . | . | 0.63 | −1.09 | . | . | F | 0.90 | 1.57 |
| Ala | 204 | A | A | . | . | . | . | . | 0.89 | −0.60 | . | * | F | 0.90 | 1.57 |
| Glu | 205 | A | A | . | . | . | . | . | 0.89 | −0.47 | . | . | F | 0.60 | 1.00 |
| Glu | 206 | A | A | . | . | . | . | . | 0.89 | −0.50 | . | . | F | 0.90 | 1.60 |
| Thr | 207 | . | A | . | . | T | . | . | 0.80 | −0.51 | . | . | F | 1.30 | 2.28 |
| Met | 208 | . | A | . | . | T | . | . | 1.18 | −0.63 | . | . | F | 1.30 | 1.76 |
| Asn | 209 | . | A | . | . | T | . | . | 1.42 | −0.20 | . | . | F | 1.00 | 1.57 |
| Thr | 210 | . | A | . | . | . | . | C | 1.11 | 0.23 | . | . | F | 0.20 | 1.08 |
| Ser | 211 | . | . | . | . | . | T | C | 0.90 | 0.23 | . | . | F | 0.60 | 1.57 |
| Pro | 212 | . | . | . | . | T | T | . | 0.62 | 0.04 | . | . | F | 0.80 | 1.51 |
| Gly | 213 | . | . | . | . | . | T | C | 1.01 | 0.14 | . | . | F | 0.60 | 1.06 |
| Thr | 214 | . | . | . | . | . | T | C | 0.42 | 0.09 | . | . | F | 0.60 | 1.22 |
| Pro | 215 | . | . | . | . | . | . | C | 0.14 | 0.20 | . | . | F | 0.25 | 0.80 |
| Ala | 216 | . | A | . | . | . | . | C | 0.44 | 0.27 | . | . | F | 0.05 | 0.82 |
| Pro | 217 | . | A | . | . | . | . | C | 0.66 | −0.16 | . | . | . | 0.50 | 0.98 |
| Ala | 218 | . | A | . | . | . | . | C | 0.69 | −0.64 | . | . | . | 0.95 | 1.10 |
| Ala | 219 | A | A | . | . | . | . | . | 0.40 | −0.59 | * | . | . | 0.75 | 1.57 |
| Glu | 220 | A | A | . | . | . | . | . | 0.61 | −0.47 | * | . | F | 0.60 | 1.00 |
| Glu | 221 | A | A | . | . | . | . | . | 0.89 | −0.50 | * | . | F | 0.90 | 1.60 |
| Thr | 222 | . | A | . | . | T | . | . | 0.80 | −0.51 | . | . | F | 1.30 | 2.28 |
| Met | 223 | . | A | . | . | T | . | . | 1.18 | −0.63 | . | . | F | 1.30 | 1.76 |
| Asn | 224 | . | A | . | . | T | . | . | 1.42 | −0.20 | . | . | F | 1.00 | 1.57 |
| Thr | 225 | . | A | . | . | . | . | C | 1.11 | 0.23 | . | . | F | 0.20 | 1.08 |
| Ser | 226 | . | . | . | . | . | T | C | 0.90 | 0.23 | . | . | F | 0.60 | 1.57 |
| Pro | 227 | . | . | . | . | T | T | . | 0.62 | 0.04 | . | . | F | 0.80 | 1.51 |
| Gly | 228 | . | . | . | . | . | T | C | 1.01 | 0.14 | . | . | F | 0.60 | 1.06 |
| Thr | 229 | . | . | . | . | . | T | C | 0.42 | 0.09 | . | . | F | 0.60 | 1.22 |
| Pro | 230 | . | . | . | . | . | . | C | 0.14 | 0.20 | . | . | F | 0.25 | 0.80 |
| Ala | 231 | . | A | . | . | . | . | C | 0.44 | 0.27 | . | . | F | 0.05 | 0.82 |
| Pro | 232 | . | A | . | . | . | . | C | 0.66 | −0.16 | . | . | . | 0.50 | 0.98 |
| Ala | 233 | . | A | . | . | . | . | C | 0.69 | −0.64 | . | . | . | 0.95 | 1.10 |
| Ala | 234 | A | A | . | . | . | . | . | 0.40 | −0.59 | . | . | . | 0.75 | 1.57 |
| Glu | 235 | A | A | . | . | . | . | . | 0.30 | −0.47 | . | . | F | 0.60 | 1.00 |
| Glu | 236 | A | A | . | . | . | . | . | 0.58 | −0.41 | . | . | F | 0.60 | 1.43 |
| Thr | 237 | . | A | B | . | . | . | . | 0.49 | −0.43 | . | . | F | 0.60 | 2.05 |
| Met | 238 | . | A | . | . | T | . | . | 0.87 | −0.54 | . | . | F | 1.30 | 1.58 |
| Thr | 239 | . | A | . | . | T | . | . | 1.11 | −0.11 | . | . | F | 1.00 | 1.41 |
| Thr | 240 | . | A | . | . | . | . | C | 0.80 | 0.31 | . | . | F | 0.05 | 0.97 |
| Ser | 241 | . | . | . | . | . | T | C | 0.59 | 0.31 | . | . | F | 0.60 | 1.41 |
| Pro | 242 | . | . | . | . | T | T | . | 0.31 | 0.13 | . | . | F | 0.80 | 1.51 |
| Gly | 243 | . | . | . | . | . | T | C | 0.70 | 0.14 | . | . | F | 0.60 | 1.06 |
| Thr | 244 | . | . | . | . | . | T | C | 0.42 | 0.09 | . | . | F | 0.60 | 1.22 |
| Pro | 245 | . | . | . | . | . | . | C | 0.14 | 0.20 | . | . | F | 0.25 | 0.80 |
| Ala | 246 | . | A | . | . | . | . | C | 0.44 | 0.27 | . | . | F | 0.05 | 0.82 |
| Pro | 247 | . | A | . | . | . | . | C | 0.66 | −0.16 | . | . | . | 0.50 | 0.98 |
| Ala | 248 | . | A | . | . | . | . | C | 0.69 | −0.64 | . | . | . | 0.95 | 1.10 |
| Ala | 249 | A | A | . | . | . | . | . | 0.40 | −0.59 | . | . | . | 0.75 | 1.57 |
| Glu | 250 | A | A | . | . | . | . | . | 0.30 | −0.47 | . | . | F | 0.60 | 1.00 |
| Glu | 251 | A | A | . | . | . | . | . | 0.58 | −0.41 | . | . | F | 0.60 | 1.43 |
| Thr | 252 | . | A | B | . | . | . | . | 0.49 | −0.43 | . | . | F | 0.60 | 2.05 |
| Met | 253 | . | A | . | . | T | . | . | 0.87 | −0.54 | . | . | F | 1.30 | 1.58 |
| Thr | 254 | . | A | . | . | T | . | . | 1.11 | −0.11 | . | . | F | 1.00 | 1.41 |
| Thr | 255 | . | A | . | . | . | . | C | 0.80 | 0.31 | . | . | F | 0.05 | 0.97 |
| Ser | 256 | . | . | . | . | . | T | C | 0.59 | 0.31 | . | . | F | 0.60 | 1.41 |
| Pro | 257 | . | . | . | . | T | T | . | 0.31 | 0.13 | . | . | F | 0.80 | 1.51 |
| Gly | 258 | . | . | . | . | . | T | C | 0.70 | 0.14 | . | . | F | 0.60 | 1.06 |
| Thr | 259 | . | . | . | . | . | T | C | 0.42 | 0.09 | . | . | F | 0.60 | 1.22 |
| Pro | 260 | . | . | . | . | . | . | C | 0.14 | 0.20 | . | . | F | 0.25 | 0.80 |
| Ala | 261 | . | A | . | . | . | . | C | 0.44 | 0.27 | . | . | F | 0.05 | 0.82 |
| Pro | 262 | . | A | . | . | . | . | C | 0.66 | −0.16 | . | . | . | 0.50 | 0.98 |
| Ala | 263 | . | A | . | . | . | . | C | 0.69 | −0.64 | . | . | . | 0.95 | 1.10 |
| Ala | 264 | A | A | . | . | . | . | . | 0.40 | −0.59 | . | . | . | 0.75 | 1.57 |
| Glu | 265 | A | A | . | . | . | . | . | 0.30 | −0.47 | . | . | F | 0.60 | 1.00 |
| Glu | 266 | A | A | . | . | . | . | . | 0.58 | −0.41 | . | . | F | 0.60 | 1.43 |
| Thr | 267 | . | A | B | . | . | . | . | 0.49 | −0.43 | . | . | F | 0.60 | 2.05 |
| Met | 268 | . | A | . | . | T | . | . | 0.87 | −0.54 | . | . | F | 1.30 | 1.58 |
| Thr | 269 | . | A | . | . | T | . | . | 1.11 | −0.11 | . | . | F | 1.00 | 1.41 |
| Thr | 270 | . | A | . | . | . | . | C | 0.80 | 0.31 | . | . | F | 0.05 | 0.97 |
| Ser | 271 | . | . | . | . | . | T | C | 0.59 | 0.31 | . | . | F | 0.60 | 1.41 |
| Pro | 272 | . | . | . | . | . | T | C | 0.31 | 0.13 | . | . | F | 0.60 | 1.51 |
| Gly | 273 | . | . | . | . | . | T | C | 0.61 | 0.14 | . | . | F | 0.60 | 1.06 |
| Thr | 274 | . | . | . | . | . | T | C | 0.62 | 0.04 | . | . | F | 0.60 | 1.06 |
| Pro | 275 | . | . | . | . | . | . | C | 0.90 | 0.04 | . | . | F | 0.25 | 0.92 |
| Ala | 276 | . | . | . | . | T | . | . | 0.96 | 0.11 | . | . | F | 0.60 | 1.26 |
| Ser | 277 | . | . | . | . | T | T | . | 0.36 | 0.44 | . | . | F | 0.50 | 1.37 |
| Ser | 278 | . | . | . | . | T | T | . | 0.40 | 0.64 | . | . | . | 0.20 | 0.73 |
| His | 279 | . | . | . | . | T | T | . | 0.04 | 0.60 | . | . | . | 0.20 | 0.97 |
| Tyr | 280 | . | . | . | . | T | T | . | −0.06 | 0.67 | . | * | . | 0.20 | 0.39 |
| Leu | 281 | . | . | . | B | T | . | . | −0.36 | 0.77 | . | . | . | −0.20 | 0.42 |
| Ser | 282 | . | . | . | B | T | . | . | −0.91 | 1.07 | . | . | . | −0.20 | 0.22 |
| Cys | 283 | . | . | B | B | . | . | . | −0.96 | 1.21 | . | . | . | −0.60 | 0.10 |
| Thr | 284 | . | . | B | B | . | . | . | −1.81 | 0.89 | * | . | . | −0.60 | 0.12 |
| Ile | 285 | . | . | B | B | . | . | . | −2.46 | 0.89 | . | . | . | −0.60 | 0.06 |
| Val | 286 | . | . | B | B | . | . | . | −2.50 | 1.19 | . | . | . | −0.60 | 0.08 |
| Gly | 287 | . | . | B | B | . | . | . | −3.01 | 1.26 | . | . | . | −0.60 | 0.04 |
| Ile | 288 | . | . | B | B | . | . | . | −3.23 | 1.46 | * | . | . | −0.60 | 0.05 |
| Ile | 289 | . | . | B | B | . | . | . | −3.78 | 1.46 | . | . | . | −0.60 | 0.05 |
| Val | 290 | . | . | B | B | . | . | . | −3.70 | 1.46 | . | . | . | −0.60 | 0.04 |
| Leu | 291 | . | . | B | B | . | . | . | −3.66 | 1.71 | . | . | . | −0.60 | 0.04 |
| Ile | 292 | . | . | B | B | . | . | . | −4.20 | 1.71 | . | . | . | −0.60 | 0.05 |
| Val | 293 | . | . | B | B | . | . | . | −4.17 | 1.71 | . | . | . | −0.60 | 0.05 |
| Leu | 294 | . | . | B | B | . | . | . | −3.98 | 1.71 | . | . | . | −0.60 | 0.04 |
| Leu | 295 | . | . | B | B | . | . | . | −3.98 | 1.81 | . | . | . | −0.60 | 0.05 |
| Ile | 296 | . | . | B | B | . | . | . | −3.56 | 1.77 | . | . | . | −0.60 | 0.05 |
| Val | 297 | . | . | B | B | . | . | . | −3.06 | 1.56 | . | . | . | −0.60 | 0.08 |
| Phe | 298 | . | . | B | B | . | . | . | −2.59 | 1.30 | . | . | . | −0.60 | 0.13 |
| Val | 299 | . | . | B | B | . | . | . | −2.17 | 1.04 | . | . | . | −0.60 | 0.23 |

In another aspect, the invention provides an antibody that binds a peptide or polypeptide comprising, or alternatively, consisting of, one, two, three, four, five or more, epitope-bearing portions of TR5. The epitope of this polypeptide portion is an immunogenic or antigenic epitope of a polypeptide of the invention. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., et al., “Antibodies That React With Predetermined Sites on Proteins,” Science, 219:660-666 (1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides of the invention are therefore useful to raise antibodies, including monoclonal antibodies, that bind to a TR5 polypeptide. See, for instance, Wilson et al., Cell 37:767-778 (1984) at 777. Antigenic epitope-bearing peptides and polypeptides preferably contain a sequence of at least seven, more preferably at least nine and most preferably between about 15 to about 30 amino acids contained within the amino acid sequence of a polypeptide of the invention.

Antibodies of the invention may bind one or more antigenic TR5 polypeptides or peptides including, but not limited to: a polypeptide comprising amino acid residues from about Gln-82 to about Glu-92 SEQ ID NO:2; a polypeptide comprising amino acid residues from about His-98 to about Cys-106 in SEQ ID NO:2; a polypeptide comprising amino acid residues from about Gln-82 to about Thr-Cys-106 in SEQ ID NO:2; a polypeptide comprising amino acid residues from about Pro-108 to about Thr-116 in SEQ ID NO:2; a polypeptide comprising amino acid residues from about Ser-119 to about Cys-125 in SEQ ID NO:2; a polypeptide comprising amino acid residues from about Cys-131 to about Thr-142 in SEQ ID NO:2; a polypeptide comprising amino acid residues from about Gln-150 to about Pro-162 in SEQ ID NO:2; a polypeptide comprising amino acid residues from about Arg-166 to about Val-176 in SEQ ID NO:2; a polypeptide comprising amino acid residues from about Gln-150 to about Val-176 in SEQ ID NO:2; and a polypeptide comprising amino acid residues from about Thr-182 to about Gln-188 in SEQ ID NO:2) As indicated above, the inventors have determined that the above polypeptide fragments are antigenic regions of the TR5 protein. In this context “about” includes the particularly recited ranges, larger or smaller by several (5, 4, 3, 2, or 1) nucleotides, at either terminus or at both termini. As indicated above, the inventors have determined that the above polypeptide fragments are antigenic regions of the TR5 receptor protein.

The epitope-bearing TR5 peptides and polypeptides may be produced by any conventional means (See, e.g., Houghten, R. A. “General method for the rapid solid-phase synthesis of large numbers of peptides: specificity of antigen-antibody interaction at the level of individual amino acids.” Proc. Natl. Acad. Sci. USA 82:5131-5135 (1985); this “Simultaneous Multiple Peptide Synthesis (SMPS)” process is further described in U.S. Pat. No. 4,631,211 to Houghten et al. (1986)).

Epitope-bearing peptides and polypeptides of the invention are used to induce antibodies according to methods well known in the art. See, for instance, Sutcliffe et al., supra; Wilson et al., supra; Chow, M et al., Proc. Natl. Acad. Sci. USA 82:910-914; and Bittle, F. J. et al., J. Gen. Virol. 66:2347-2354 (1985). Immunogenic epitope-bearing peptides of the invention, i.e., those parts of a protein that elicit an antibody response when the whole protein is the immunogen, are identified according to methods known in the art. See, for instance, Geysen et al., supra. Further still, U.S. Pat. No. 5,194,392 to Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 to Geysen (1989) describes a method of detecting or determining a sequence of monomers which is a topographical equivalent of a ligand which is complementary to the ligand binding site of a particular receptor of interest. Similarly, U.S. Pat. No. 5,480,971 to Houghten, R. A. et al. (1996) on Peralkylated Oligopeptide Mixtures discloses linear C1-C7-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

As one of skill in the art will appreciate, TR5 receptor polypeptides of the present invention and the epitope-bearing fragments thereof described herein above (e.g., corresponding to a portion of the extracellular domain, such as, for example, polypeptide sequence comprising, or alternatively, consisting of, amino acid residues 41 to 280, 67 to 280, 70 to 280, 75 to 280, 80 to 280 and 90 to 280 of SEQ ID NO:2) can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EP A 394,827; Traunecker et al., Nature 331:84-86 (1988)). Fusion proteins that have a disulfide-linked dimeric structure due to the IgG part can also be more efficient in binding and neutralizing other molecules than the monomeric TR5 protein or protein fragment alone (Fountoulakis et al., J. Biochem. 270:3958-3964 (1995)). Thus, antibodies of the invention may bind fusion proteins that comprise all or a portion of a TRAIL receptor polypeptide such as TR5.

Recombinant DNA technology known to those skilled in the art can be used to create novel mutant proteins or “muteins” including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Antibodies of the present invention may also bind such modified TR5 polypeptides or TR5 polypeptide fragments or variants.

For instance, for many proteins, including the extracellular domain of a membrane associated protein or the mature form(s) of a secreted protein, it is known in the art that one or more amino acids may be deleted from the N-terminus or C-terminus without substantial loss of biological function or loss of the ability to be bound by a specific antibody. For instance, Ron et al., J. Biol. Chem., 268:2984-2988 (1993) reported modified KGF proteins that had heparin binding activity even if 3, 8, or 27 amino-terminal amino acid residues were missing. In the present case, since the proteins of the invention are members of the TNFR polypeptide family, deletions of N-terminal amino acids up to the cysteine at position C-93 of SEQ ID NO:2 may retain some biological activity such as regulation of proliferation and apoptosis of lymphoid cells. Polypeptides having further N-terminal deletions including the C-93 residue of SEQ ID NO:2, would not be expected to retain such biological activities because it is known that these residues in a TR5-related polypeptide are required for forming a disulfide bridge to provide structural stability which is needed for ligand binding. Thus, in specific embodiments, antibodies of the present invention bind N-terminally deleted protein fragments which retain biological activity.

As mentioned above, even if deletion of one or more amino acids from the N-terminus of a protein results in modification or loss of one or more biological functions of the protein, other functional activities (e.g., biological activities, ability to multimerize, ability to bind TRAIL ligand) may still be retained. For example, the ability of the shortened protein to induce and/or bind to antibodies which recognize the complete or mature form of the TR5 protein generally will be retained when less than the majority of the residues of the complete protein or extracellular domain are removed from the N-terminus. Whether a particular polypeptide lacking N-terminal residues of a complete protein retains such immunologic activities can readily be determined by routine methods described herein and otherwise known in the art. It is not unlikely that a TR5 mutein with a large number of deleted N-terminal amino acid residues may retain some biological or immunogenic activities. In fact, peptides composed of as few as six TR5 amino acid residues may often evoke an immune response.

Accordingly, the present invention further provides antibodies that bind TR5 polypeptides having one or more residues deleted from the amino terminus of TR5 (SEQ ID NO:2), up to the cysteine residue in each which is at position number 93, and polynucleotides encoding such polypeptides. In particular, the present invention provides antibodies that bind TR5 polypeptides comprising the amino acid sequence of residues n³-299 of SEQ ID NO:2 where n³ is an integer in the range of 1-93 where 93 is the position of the first cysteine residue from the N-terminus of the complete TR5 polypeptide believed to be required for activity of the TR5 protein.

More in particular, the invention provides antibodies that bind polypeptides having the amino acid sequence of residues: Q-2 to V-299; G-3 to V-299; V-4 to V-299; K-5 to V-299; E-6 to V-299; R-7 to V-299; F-8 to V-299; L-9 to V-299; P-10 to V-299; L-11 to V-299; G-12 to V-299; N-13 to V-299; S-14 to V-299; G-15 to V-299; D-16 to V-299; R-17 to V-299; A-18 to V-299; P-19 to V-299; R-20 to V-299; P-21 to V-299; P-22 to V-299; D-23 to V-299; G-24 to V-299; R-25 to V-299; G-26 to V-299; R-27 to V-299; V-28 to V-299; R-29 to V-299; P-30 to V-299; R-31 to V-299; T-32 to V-299; Q-33 to V-299; D-34 to V-299; G-35 to V-299; V-36 to V-299; G-37 to V-299; N-38 to V-299; H-39 to V-299; T-40 to V-299; M-41 to V-299; A-42 to V-299; R-43 to V-299; I-44 to V-299; P-45 to V-299; K-46 to V-299; T-47 to V-299; L-48 to V-299; K-49 to V-299; F-50 to V-299; V-51 to V-299; V-52 to V-299; V-53 to V-299; I-54 to V-299; V-55 to V-299; A-56 to V-299; V-57 to V-299; L-58 to V-299; L-59 to V-299; P-60 to V-299; V-61 to V-299; L-62 to V-299; A-63 to V-299; Y-64 to V-299; S-65 to V-299; A-66 to V-299; T-67 to V-299; T-68 to V-299; A-69 to V-299; R-70 to V-299; Q-71 to V-299; E-72 to V-299; E-73 to V-299; V-74 to V-299; P-75 to V-299; Q-76 to V-299; Q-77 to V-299; T-78 to V-299; V-79 to V-299; A-80 to V-299; P-81 to V-299; Q-82 to V-299; Q-83 to V-299; Q-84 to V-299; R-85 to V-299; H-86 to V-299; S-87 to V-299; F-88 to V-299; K-89 to V-299; G-90 to V-299; E-91 to V-299; E-92 to V-299; and/or C-93 to V-299 of SEQ ID NO:2.

Similarly, the present invention further provides antibodies that bind polypeptides having one or more residues deleted from the amino terminus of the TR5 amino acid sequence shown in SEQ ID NO:2, up to the leucine residue at position number 295. In particular, the present invention provides antibodies that bind polypeptides comprising the amino acid sequence of residues n⁴-299 of SEQ ID NO:2, where n⁴ is an integer from 2 to 294 corresponding to the position of the amino acid residue in SEQ ID NO:2.

More in particular, the invention provides antibodies that bind polypeptides comprising, or alternatively consisting of, the amino acid sequence of residues: Q-2 to V-299; G-3 to V-299; V-4 to V-299; K-5 to V-299; E-6 to V-299; R-7 to V-299; F-8 to V-299; L-9 to V-299; P-10 to V-299; L-11 to V-299; G-12 to V-299; N-13 to V-299; S-14 to V-299; G-15 to V-299; D-16 to V-299; R-17 to V-299; A-18 to V-299; P-19 to V-299; R-20 to V-299; P-21 to V-299; P-22 to V-299; D-23 to V-299; G-24 to V-299; R-25 to V-299; G-26 to V-299; R-27 to V-299; V-28 to V-299; R-29 to V-299; P-30 to V-299; R-31 to V-299; T-32 to V-299; Q-33 to V-299; D-34 to V-299; G-35 to V-299; V-36 to V-299; G-37 to V-299; N-38 to V-299; H-39 to V-299; T-40 to V-299; M-41 to V-299; A-42 to V-299; R-43 to V-299; I-44 to V-299; P-45 to V-299; K-46 to V-299; T-47 to V-299; L-48 to V-299; K-49 to V-299; F-50 to V-299; V-51 to V-299; V-52 to V-299; V-53 to V-299; I-54 to V-299; V-55 to V-299; A-56 to V-299; V-57 to V-299; L-58 to V-299; L-59 to V-299; P-60 to V-299; V-61 to V-299; L-62 to V-299; A-63 to V-299; Y-64 to V-299; S-65 to V-299; A-66 to V-299; T-67 to V-299; T-68 to V-299; A-69 to V-299; R-70 to V-299; Q-71 to V-299; E-72 to V-299; E-73 to V-299; V-74 to V-299; P-75 to V-299; Q-76 to V-299; Q-77 to V-299; T-78 to V-299; V-79 to V-299; A-80 to V-299; P-81 to V-299; Q-82 to V-299; Q-83 to V-299; Q-84 to V-299; R-85 to V-299; H-86 to V-299; S-87 to V-299; F-88 to V-299; K-89 to V-299; G-90 to V-299; E-91 to V-299; E-92 to V-299; C-93 to V-299; P-94 to V-299; A-95 to V-299; G-96 to V-299; S-97 to V-299; H-98 to V-299; R-99 to V-299; S-100 to V-299; E-101 to V-299; H-102 to V-299; T-103 to V-299; G-104 to V-299; A-105 to V-299; C-106 to V-299; N-107 to V-299; P-108 to V-299; C-109 to V-299; T-110 to V-299; E-111 to V-299; G-112 to V-299; V-113 to V-299; D-114 to V-299; Y-115 to V-299; T-116 to V-299; N-117 to V-299; A-118 to V-299; S-119 to V-299; N-120 to V-299; N-121 to V-299; E-122 to V-299; P-123 to V-299; S-124 to V-299; C-125 to V-299; F-126 to V-299; P-127 to V-299; C-128 to V-299; T-129 to V-299; V-130 to V-299; C-131 to V-299; K-132 to V-299; S-133 to V-299; D-134 to V-299; Q-135 to V-299; K-136 to V-299; H-137 to V-299; K-138 to V-299; S-139 to V-299; S-140 to V-299; C-141 to V-299; T-142 to V-299; M-143 to V-299; T-144 to V-299; R-145 to V-299; D-146 to V-299; T-147 to V-299; V-148 to V-299; C-149 to V-299; Q-150 to V-299; C-151 to V-299; K-152 to V-299; E-153 to V-299; G-154 to V-299; T-155 to V-299; F-156 to V-299; R-157 to V-299; N-158 to V-299; E-159 to V-299; N-160 to V-299; S-161 to V-299; P-162 to V-299; E-163 to V-299; M-164 to V-299; C-165 to V-299; R-166 to V-299; K-167 to V-299; C-168 to V-299; S-169 to V-299; R-170 to V-299; C-171 to V-299; P-172 to V-299; S-173 to V-299; G-174 to V-299; E-175 to V-299; V-176 to V-299; Q-177 to V-299; V-178 to V-299; S-179 to V-299; N-180 to V-299; C-181 to V-299; T-182 to V-299; S-183 to V-299; W-184 to V-299; D-185 to V-299; D-186 to V-299; I-187 to V-299; Q-188 to V-299; C-189 to V-299; V-190 to V-299; E-191 to V-299; E-192 to V-299; F-193 to V-299; G-194 to V-299; A-195 to V-299; N-196 to V-299; A-197 to V-299; T-198 to V-299; V-199 to V-299; E-200 to V-299; T-201 to V-299; P-202 to V-299; A-203 to V-299; A-204 to V-299; E-205 to V-299; E-206 to V-299; T-207 to V-299; M-208 to V-299; N-209 to V-299; T-210 to V-299; S-211 to V-299; P-212 to V-299; G-213 to V-299; T-214 to V-299; P-215 to V-299; A-216 to V-299; P-217 to V-299; A-218 to V-299; A-219 to V-299; E-220 to V-299; E-221 to V-299; T-222 to V-299; M-223 to V-299; N-224 to V-299; T-225 to V-299; S-226 to V-299; P-227 to V-299; G-228 to V-299; T-229 to V-299; P-230 to V-299; A-231 to V-299; P-232 to V-299; A-233 to V-299; A-234 to V-299; E-235 to V-299; E-236 to V-299; T-237 to V-299; M-238 to V-299; T-239 to V-299; T-240 to V-299; S-241 to V-299; P-242 to V-299; G-243 to V-299; T-244 to V-299; P-245 to V-299; A-246 to V-299; P-247 to V-299; A-248 to V-299; A-249 to V-299; E-250 to V-299; E-251 to V-299; T-252 to V-299; M-253 to V-299; T-254 to V-299; T-255 to V-299; S-256 to V-299; P-257 to V-299; G-258 to V-299; T-259 to V-299; P-260 to V-299; A-261 to V-299; P-262 to V-299; A-263 to V-299; A-264 to V-299; E-265 to V-299; E-266 to V-299; T-267 to V-299; M-268 to V-299; T-269 to V-299; T-270 to V-299; S-271 to V-299; P-272 to V-299; G-273 to V-299; T-274 to V-299; P-275 to V-299; A-276 to V-299; S-277 to V-299; S-278 to V-299; H-279 to V-299; Y-280 to V-299; L-281 to V-299; S-282 to V-299; C-283 to V-299; T-284 to V-299; I-285 to V-299; V-286 to V-299; G-287 to V-299; I-288 to V-299; I-289 to V-299; V-290 to V-299; L-291 to V-299; I-292 to V-299; V-293 to V-299; and/or L-294 to V-299 of the TR5 sequence shown in SEQ ID NO:2.

Similarly, many examples of biologically functional C-terminal deletion muteins are known. For instance, interferon gamma shows up to ten times higher activities by deleting 8-10 amino acid residues from the carboxy terminus of the protein (Dobeli et al., J. Biotechnology 7:199-216 (1988)). In the present case, since the protein of the invention is a member of the TNFR polypeptide family, deletions of C-terminal amino acids up to the cysteine at position 189 of SEQ ID NO:2, may retain some biological activity such as regulation of proliferation and apoptosis of lymphoid cells. Polypeptides having further C-terminal deletions including the cysteine at position 189 of SEQ ID NO:2 would not be expected to retain such biological activities because it is known that this residue in TNF receptor-related polypeptides is required for forming a disulfide bridge to provide structural stability which is needed for ligand binding.

Also as mentioned above, even if deletion of one or more amino acids from the C-terminus of a protein results in modification or loss of one or more biological functions of the protein, other functional activities (e.g., biological activities, ability to multimerize, ability to bind TR5 ligand (e.g., TRAIL)) may still be retained. Thus, the ability of the shortened protein to induce and/or bind to antibodies which recognize the complete or mature form of the protein generally will be retained when less than the majority of the residues of the complete or mature form protein are removed from the C-terminus. Whether a particular polypeptide lacking C-terminal residues of a complete protein retains such immunologic activities can readily be determined by routine methods described herein and otherwise known in the art. It is not unlikely that a TR5 polypeptide with a large number of deleted C-terminal amino acid residues may retain some biological or immunogenic activities. In fact, peptides composed of as few as six TR5 amino acid residues may often evoke an immune response.

Accordingly, the present invention further provides antibodies that bind polypeptides having one or more residues from the carboxy terminus of the amino acid sequence of TR5 shown in SEQ ID NO:2 up to the cysteine at position 189 of SEQ ID NO:2. In particular, the present invention provides antibodies that bind polypeptides having the amino acid sequence of residues 1-x of the amino acid sequence in SEQ ID NO:2, where x is any integer in the range of 189-299.

The present invention further provides antibodies that bind polypeptides having one or more residues deleted from the carboxy terminus of the amino acid sequence of the TR5 polypeptide shown in SEQ ID NO:2 up to the glutamine residue at position number at position number 6. In particular, the present invention provides antibodies that bind polypeptides comprising the amino acid sequence of residues 1-m³ of SEQ ID NO:3, where m³ is an integer from 6 to 298 corresponding to the position of the amino acid residue in SEQ ID NO:3.

More in particular, the invention provides antibodies that bind polypeptides comprising, or alternatively consisting of, the amino acid sequence of residues: M-1 to F-298; M-1 to V-297; M-1 to I-296; M-1 to L-295; M-1 to L-294; M-1 to V-293; M-1 to I-292; M-1 to L-291; M-1 to V-290; M-1 to I-289; M-1 to I-288; M-1 to G-287; M-1 to V-286; M-1 to I-285; M-1 to T-284; M-1 to C-283; M-1 to S-282; M-1 to L-281; M-1 to Y-280; M-1 to H-279; M-1 to S-278; M-1 to S-277; M-1 to A-276; M-1 to P-275; M-1 to T-274; M-1 to G-273; M-1 to P-272; M-1 to S-271; M-1 to T-270; M-1 to T-269; M-1 to M-268; M-1 to T-267; M-1 to E-266; M-1 to E-265; M-1 to A-264; M-1 to A-263; M-1 to P-262; M-1 to A-261; M-1 to P-260; M-1 to T-259; M-1 to G-258; M-1 to P-257; M-1 to S-256; M-1 to T-255; M-1 to T-254; M-1 to M-253; M-1 to T-252; M-1 to E-251; M-1 to E-250; M-1 to A-249; M-1 to A-248; M-1 to P-247; M-1 to A-246; M-1 to P-245; M-1 to T-244; M-1 to G-243; M-1 to P-242; M-1 to S-241; M-1 to T-240; M-1 to T-239; M-1 to M-238; M-1 to T-237; M-1 to E-236; M-1 to E-235; M-1 to A-234; M-1 to A-233; M-1 to P-232; M-1 to A-231; M-1 to P-230; M-1 to T-229; M-1 to G-228; M-1 to P-227; M-1 to S-226; M-1 to T-225; M-1 to N-224; M-1 to M-223; M-1 to T-222; M-1 to E-221; M-1 to E-220; M-1 to A-219; M-1 to A-218; M-1 to P-217; M-1 to A-216; M-1 to P-215; M-1 to T-214; M-1 to G-213; M-1 to P-212; M-1 to S-211; M-1 to T-210; M-1 to N-209; M-1 to M-208; M-1 to T-207; M-1 to E-206; M-1 to E-205; M-1 to A-204; M-1 to A-203; M-1 to P-202; M-1 to T-201; M-1 to E-200; M-1 to V-199; M-1 to T-198; M-1 to A-197; M-1 to N-196; M-1 to A-195; M-1 to G-194; M-1 to F-193; M-1 to E-192; M-1 to E-191; M-1 to V-190; M-1 to C-189; M-1 to Q-188; M-1 to I-187; M-1 to D-186; M-1 to D-185; M-1 to W-184; M-1 to S-183; M-1 to T-182; M-1 to C-181; M-1 to N-180; M-1 to S-179; M-1 to V-178; M-1 to Q-177; M-1 to V-176; M-1 to E-175; M-1 to G-174; M-1 to S-173; M-1 to P-172; M-1 to C-171; M-1 to R-170; M-1 to S-169; M-1 to C-168; M-1 to K-167; M-1 to R-166; M-1 to C-165; M-1 to M-164, M-1 to E-163; M-1 to P-162; M-1 to S-161; M-1 to N-160; M-1 to E-159; M-1 to N-158; M-1 to R-157; M-1 to F-156; M-1 to T-155; M-1 to G-154; M-1 to E-153; M-1 to K-152; M-1 to C-151; M-1 to Q-150; M-1 to C-149; M-1 to V-148; M-1 to T-147; M-1 to D-146; M-1 to R-145; M-1 to T-144; M-1 to M-143; M-1 to T-142; M-1 to C-141; M-1 to S-140; M-1 to S-139; M-1 to K-138; M-1 to H-137; M-1 to K-136; M-1 to Q-135; M-1 to D-134; M-1 to S-133; M-1 to K-132; M-1 to C-131; M-1 to V-130; M-1 to T-129; M-1 to C-128; M-1 to P-127; M-1 to F-126; M-1 to C-125; M-1 to S-124; M-1 to P-123; M-1 to E-122; M-1 to N-121; M-1 to N-120; M-1 to S-19; M-1 to A-118; M-1 to N-117; M-1 to T-116; M-1 to Y-115; M-1 to D-114; M-1 to V-113; M-1 to G-112; M-1 to E-111; M-1 to T-110; M-1 to C-109; M-1 to P-108; M-1 to N-107; M-1 to C-106; M-1 to A-105; M-1 to G-104; M-1 to T-103; M-1 to H-102; M-1 to E-101; M-1 to S-100; M-1 to R-99; M-1 to H-98; M-1 to S-97; M-1 to G-96; M-1 to A-95; M-1 to P-94; M-1 to C-93; M-1 to E-92; M-1 to E-91; M-1 to G-90; M-1 to K-89; M-1 to F-88; M-1 to S-87; M-1 to H-86; M-1 to R-85; M-1 to Q-84; M-1 to Q-83; M-1 to Q-82; M-1 to P-81; M-1 to A-80; M-1 to V-79; M-1 to T-78; M-1 to Q-77; M-1 to Q-76; M-1 to P-75; M-1 to V-74; M-1 to E-73; M-1 to E-72; M-1 to Q-71; M-1 to R-70; M-1 to A-69; M-1 to T-68; M-1 to T-67; M-1 to A-66; M-1 to S-65; M-1 to Y-64; M-1 to A-63; M-1 to L-62; M-1 to V-61; M-1 to P-60; M-1 to L-59; M-1 to L-58; M-1 to V-57; M-1 to A-56; M-1 to V-55; M-1 to I-54; M-1 to V-53; M-1 to V-52; M-1 to V-51; M-1 to F-50; M-1 to K-49; M-1 to L-48; M-1 to T-47; M-1 to K-46; M-1 to P-45; M-1 to 144; M-1 to R-43; M-1 to A-42; M-1 to M-41; M-1 to T-40; M-1 to H-39; M-1 to N-38; M-1 to G-37; M-1 to V-36; M-1 to G-35; M-1 to D-34; M-1 to Q-33; M-1 to T-32; M-1 to R-31; M-1 to P-30; M-1 to R-29; M-1 to V-28; M-1 to R-27; M-1 to G-26; M-1 to R-25; M-1 to G-24; M-1 to D-23; M-1 to P-22; M-1 to P-21; M-1 to R-20; M-1 to P-19; M-1 to A-18; M-1 to R-17; M-1 to D-16; M-1 to G-15; M-1 to S-14; M-1 to N-13; M-1 to G-12; M-1 to L-11; M-1 to P-10; M-1 to L-9; M-1 to F-8; and/or M-1 to R-7 of the TR5 sequence shown in SEQ ID NO:2.

In another embodiment, antibodies of the invention bind C-terminal deletions of the extracellular domain of the TR5 polypeptide that can be described by the general formula 67-m⁴ where m⁴ is a number from 73 to 299 corresponding to the amino acid sequence identified in SEQ ID NO:2. In specific embodiments, antibodies of the invention bind C terminal deletions of the TR5 polypeptide comprising, or alternatively, consisting of, amino acid residues: T-67 to F-298; T-67 to V-297; T-67 to I-296; T-67 to L-295; T-67 to L-294; T-67 to V-293; T-67 to I-292; T-67 to L-291; T-67 to V-290; T-67 to I-289; T-67 to I-288; T-67 to G-287; T-67 to V-286; T-67 to I-285; T-67 to T-284; T-67 to C-283; T-67 to S-282; T-67 to L-281; T-67 to Y-280; T-67 to H-279; T-67 to S-278; T-67 to S-277; T-67 to A-276; T-67 to P-275; T-67 to T-274; T-67 to G-273; T-67 to P-272; T-67 to S-271; T-67 to T-270; T-67 to T-269; T-67 to M-268; T-67 to T-267; T-67 to E-266; T-67 to E-265; T-67 to A-264; T-67 to A-263; T-67 to P-262; T-67 to A-261; T-67 to P-260; T-67 to T-259; T-67 to G-258; T-67 to P-257; T-67 to S-256; T-67 to T-255; T-67 to T-254; T-67 to M-253; T-67 to T-252; T-67 to E-251; T-67 to E-250; T-67 to A-249; T-67 to A-248; T-67 to P-247; T-67 to A-246; T-67 to P-245; T-67 to T-244; T-67 to G-243; T-67 to P-242; T-67 to S-241; T-67 to T-240; T-67 to T-239; T-67 to M-238; T-67 to T-237; T-67 to E-236; T-67 to E-235; T-67 to A-234; T-67 to A-233; T-67 to P-232; T-67 to A-231; T-67 to P-230; T-67 to T-229; T-67 to G-228; T-67 to P-227; T-67 to S-226; T-67 to T-225; T-67 to N-224; T-67 to M-223; T-67 to T-222; T-67 to E-221; T-67 to E-220; T-67 to A-219; T-67 to A-218; T-67 to P-217; T-67 to A-216; T-67 to P-215; T-67 to T-214; T-67 to G-213; T-67 to P-212; T-67 to S-211; T-67 to T-210; T-67 to N-209; T-67 to M-208; T-67 to T-207; T-67 to E-206; T-67 to E-205; T-67 to A-204; T-67 to A-203; T-67 to P-202; T-67 to T-201; T-67 to E-200; T-67 to V-199; T-67 to T-198; T-67 to A-197; T-67 to N-196; T-67 to A-195; T-67 to G-194; T-67 to F-193; T-67 to E-192; T-67 to E-191; T-67 to V-190; T-67 to C-189; T-67 to Q-188; T-67 to I-187; T-67 to D-186; T-67 to D-185; T-67 to W-184; T-67 to S-183; T-67 to T-182; T-67 to C-181; T-67 to N-180; T-67 to S-179; T-67 to V-178; T-67 to Q-177; T-67 to V-176; T-67 to E-175; T-67 to G-174; T-67 to S-173; T-67 to P-172; T-67 to C-171; T-67 to R-170; T-67 to S-169; T-67 to C-168; T-67 to K-167; T-67 to R-166; T-67 to C-165; T-67 to M-164; T-67 to E-163; T-67 to P-162; T-67 to S-161; T-67 to N-160; T-67 to E-159; T-67 to N-158; T-67 to R-157; T-67 to F-156; T-67 to T-155; T-67 to G-154; T-67 to E-153; T-67 to K-152; T-67 to C-151; T-67 to Q-150; T-67 to C-149; T-67 to V-148; T-67 to T-147; T-67 to D-146; T-67 to R-145; T-67 to T-144; T-67 to M-143; T-67 to T-142; T-67 to C-141; T-67 to S-140; T-67 to S-139; T-67 to K-138; T-67 to H-137; T-67 to K-136; T-67 to Q-135; T-67 to D-134; T-67 to S-133; T-67 to K-132; T-67 to C-131; T-67 to V-130; T-67 to T-129; T-67 to C-128; T-67 to P-127; T-67 to F-126; T-67 to C-125; T-67 to S-124; T-67 to P-123; T-67 to E-122; T-67 to N-121; T-67 to N-120; T-67 to S-119; T-67 to A-118; T-67 to N-117; T-67 to T-116; T-67 to Y-115; T-67 to D-114; T-67 to V-113; T-67 to G-112; T-67 to E-11; T-67 to T-110; T-67 to C-109; T-67 to P-108; T-67 to N-107; T-67 to C-106; T-67 to A-105; T-67 to G-104; T-67 to T-103; T-67 to H-102; T-67 to E-101; T-67 to S-100; T-67 to R-99; T-67 to H-98; T-67 to S-97; T-67 to G-96; T-67 to A-95; T-67 to P-94; T-67 to C-93; T-67 to E-92; T-67 to E-91; T-67 to G-90; T-67 to K-89; T-67 to F-88; T-67 to S-87; T-67 to H-86; T-67 to R-85; T-67 to Q-84; T-67 to Q-83; T-67 to Q-82; T-67 to P-81; T-67 to A-80; T-67 to V-79; T-67 to T-78; T-67 to Q-77; T-67 to Q-76; T-67 to P-75; T-67 to V-74; and/or T-67 to E-73 of the TR5 extracellular domain sequence shown in SEQ ID NO:2.

The invention also provides antibodies that bind polypeptides having one or more amino acids deleted from both the amino and the carboxyl termini, which may be described generally as having residues n⁴ to m³ of SEQ ID NO:2, where n⁴ and m³ are integers as described above.

Also included are antibodies that bind a polypeptide consisting of a portion of the complete TR5 amino acid sequence encoded by a cDNA clone contained in ATCC Deposit No. 97798, where this portion excludes from 1 to about 89 amino acids from the amino terminus of the complete amino acid sequence encoded by the cDNA clone contained in ATCC Deposit No. 97798, or from 1 to about 110 amino acids from the carboxy terminus of the complete amino acid sequence encoded by the cDNA clone contained in ATCC Deposit No. 97798, or any combination of the above amino terminal and carboxy terminal deletions, of the complete amino acid sequence encoded by the cDNA clone contained in ATCC Deposit No. 97798.

In addition to terminal deletion forms of the protein discussed above, it will also be recognized by one of ordinary skill in the art that some amino acid sequences of the TR5 polypeptide can be varied without significant effect on the structure or function of the proteins. If such differences in sequence are contemplated, it should be remembered that there will be critical areas on the protein which determine activity. Thus, the invention further includes variations of the TR5 polypeptide, which show substantial TR5 polypeptide activity or which include regions of TR5 protein such as the protein portions discussed below. Such mutants include deletions, insertions, inversions, repeats, and type substitutions Guidance concerning which amino acid changes are likely to be phenotypically silent can be found in Bowie, J. U. et al., “Deciphering the Message in Protein Sequences: Tolerance to Amino Acid Substitutions,” Science 247:1306-1310 (1990).

Thus, antibodies of the present invention may bind a fragment, derivative, or analog of the polypeptide of SEQ ID NO:2, or that encoded by the cDNA in ATCC deposit 97798. Such fragments, variants or derivatives may be: (i) one in which one or more of the amino acid residues are substituted with a conserved or non-conserved amino acid residue (preferably a conserved amino acid residue(s), and more preferably at least one but less than ten conserved amino acid residue(s)), and such substituted amino acid residue(s) may or may not be one encoded by the genetic code; or (ii) one in which one or more of the amino acid residues includes a substituent group; or (iii) one in which the mature or soluble extracellular polypeptide is fused with another compound, such as a compound to increase the half-life of the polypeptide (for example, polyethylene glycol); or (iv) one in which the additional amino acids are fused to the mature polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Of particular interest are substitutions of charged amino acids with another charged amino acids and with neutral or negatively charged amino acids. The latter results in proteins with reduced positive charge to improve the characteristics of the TR5 protein. The prevention of aggregation is highly desirable. Aggregation of proteins not only results in a loss of activity but can also be problematic when preparing pharmaceutical formulations, because they can be immunogenic. (Pinckard et al., Clin Exp. Immunol 2:331-340 (1967); Robbins et al., Diabetes 36:838-845 (1987); Cleland et al., Crit Rev. Therapeutic Drug Carrier Systems 10:307-377 (1993)).

The replacement of amino acids can also change the selectivity of binding of a ligand to cell surface receptors. For example, Ostade et al., Nature 361:266-268 (1993), describes certain mutations resulting in selective binding of TNF-α to only one of the two known types of TNF receptors. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al., Science 255:306-312 (1992)). Thus, the antibodies of the present invention may bind a TR5 receptor that contains one or more amino acid substitutions, deletions or additions, either from natural mutations or human manipulation.

As indicated, changes are preferably of a minor nature, such as conservative amino acid substitutions that do not significantly affect the folding or activity of the protein (see Table 3 above).

In specific embodiments, the number of substitutions, additions or deletions in the amino acid sequence of SEQ ID NO:2 and/or any of the polypeptide fragments described herein (e.g., the extracellular domain) is 75, 70, 60, 50, 40, 35, 30, 25, 20, 15, 10, 9, 8, 7, 6, 5, 4, 3, 2, 1 or 30-20, 20-15, 20-10, 15-10, 10-1, 5-10, 1-5, 1-3 or 1-2.

In specific embodiments, the antibodies of the invention bind TR5 polypeptides or fragments or variants thereof (especially a fragment comprising or alternatively consisting of, the extracellular soluble domain of TR5), that contains any one or more of the following conservative mutations in TR5: M1 replaced with A, G, I, L, S, T, or V; Q2 replaced with N; G3 replaced with A, I, L, S, T, M, or V; V4 replaced with A, G, I, L, S, T, or M; K5 replaced with H, or R; E6 replaced with D; R7 replaced with H, or K; F8 replaced with W, or Y; L9 replaced with A, G, I, S, T, M, or V; L11 replaced with A, G, I, S, T, M, or V; G12 replaced with A, I, L, S, T, M, or V; N13 replaced with Q; S14 replaced with A, G, I, L, T, M, or V; G15 replaced with A, I, L, S, T, M, or V; D16 replaced with E; R17 replaced with H, or K; A18 replaced with G, I, L, S, T, M, or V; R20 replaced with H, or K; D23 replaced with E; G24 replaced with A, I, L, S, T, M, or V; R25 replaced with H, or K; G26 replaced with A, I, L, S, T, M, or V; R27 replaced with H, or K; V28 replaced with A, G, I, L, S, T, or M; R29 replaced with H, or K; R31 replaced with H, or K; T32 replaced with A, G, I, L, S, M, or V; Q33 replaced with N; D34 replaced with E; G35 replaced with A, I, L, S, T, M, or V; V36 replaced with A, G, I, L, S, T, or M; G37 replaced with A, I, L, S, T, M, or V; N38 replaced with Q; H39 replaced with K, or R; T40 replaced with A, G, I, L, S, M, or V; M41 replaced with A, G, I, L, S, T, or V; A42 replaced with G, I, L, S, T, M, or V; R43 replaced with H, or K; I44 replaced with A, G, L, S, T, M, or V; K46 replaced with H, or R; T47 replaced with A, G, I, L, S, M, or V; L48 replaced with A, G, I, S, T, M, or V; K49 replaced with H, or R; F50 replaced with W, or Y; V51 replaced with A, G, I, L, S, T, or M; V52 replaced with A, G, I, L, S, T, or M; V53 replaced with A, G, I, L, S, T, or M; I54 replaced with A, G, L, S, T, M, or V; V55 replaced with A, G, I, L, S, T, or M; A56 replaced with G, I, L, S, T, M, or V; V57 replaced with A, G, I, L, S, T, or M; L58 replaced with A, G, I, S, T, M, or V; L59 replaced with A, G, I, S, T, M, or V; V61 replaced with A, G, I, L, S, T, or M; L62 replaced with A, G, I, S, T, M, or V; A63 replaced with G, I, L, S, T, M, or V; Y64 replaced with F, or W; S65 replaced with A, G, I, L, T, M, or V; A66 replaced with G, I, L, S, T, M, or V; T67 replaced with A, G, I, L, S, M, or V; T68 replaced with A, G, I, L, S, M, or V; A69 replaced with G, I, L, S, T, M, or V; R70 replaced with H, or K; Q71 replaced with N; E72 replaced with D; E73 replaced with D; V74 replaced with A, G, I, L, S, T, or M; Q76 replaced with N; Q77 replaced with N; T78 replaced with A, G, I, L, S, M, or V; V79 replaced with A, G, I, L, S, T, or M; A80 replaced with G, I, L, S, T, M, or V; Q82 replaced with N; Q83 replaced with N; Q84 replaced with N; R85 replaced with H, or K; H86 replaced with K, or R; S87 replaced with A, G, I, L, T, M, or V; F88 replaced with W, or Y; K89 replaced with H, or R; G90 replaced with A, I, L, S, T, M, or V; E91 replaced with D; E92 replaced with D; A95 replaced with G, I, L, S, T, M, or V; G96 replaced with A, I, L, S, T, M, or V; S97 replaced with A, G, I, L, T, M, or V; H98 replaced with K, or R; R99 replaced with H, or K; S100 replaced with A, G, I, L, T, M, or V; E101 replaced with D; H102 replaced with K, or R; T103 replaced with A, G, I, L, S, M, or V; G104 replaced with A, I, L, S, T, M, or V; A105 replaced with G, I, L, S, T, M, or V; N107 replaced with Q; T110 replaced with A, G, I, L, S, M, or V; E111 replaced with D; G112 replaced with A, I, L, S, T, M, or V; V113 replaced with A, G, L, S, T, or M; D114 replaced with E; Y115 replaced with F, or W; T116 replaced with A, G, I, L, S, M, or V; N117 replaced with Q; A118 replaced with G, I, L, S, T, M, or V; S119 replaced with A, G, I, L, T, M, or V; N120 replaced with Q; N121 replaced with Q; E122 replaced with D; S124 replaced with A, G, I, L, T, M, or V; F126 replaced with W, or Y; T129 replaced with A, G, I, L, S, M, or V; V130 replaced with A, G, I, L, S, T, or M; K132 replaced with H, or R; S133 replaced with A, G, I, L, T, M, or V; D134 replaced with E; Q135 replaced with N; K136 replaced with H, or R; H137 replaced with K, or R; K138 replaced with H, or R; S139 replaced with A, G, I, L, T, M, or V; S140 replaced with A, G, I, L, T, M, or V; T142 replaced with A, G, I, L, S, M, or V; M143 replaced with A, G, I, L, S, T, or V; T144 replaced with A, G, I, L, S, M, or V; R145 replaced with H, or K; D146 replaced with E; T147 replaced with A, G, I, L, S, M, or V; V148 replaced with A, G, I, L, S, T, or M; Q150 replaced with N; K152 replaced with H, or R; E153 replaced with D; G154 replaced with A, I, L, S, T, M, or V; T155 replaced with A, G, I, L, S, M, or V; F156 replaced with W, or Y; R157 replaced with H, or K; N158 replaced with Q; E159 replaced with D; N160 replaced with Q; S161 replaced with A, G, I, L, T, M, or V; E163 replaced with D; M164 replaced with A, G, I, L, S, T, or V; R166 replaced with H, or K; K167 replaced with H, or R; S169 replaced with A, G, I, L, T, M, or V; R170 replaced with H, or K; S173 replaced with A, G, I, L, T, M, or V; G174 replaced with A, I, L, S, T, M, or V; E175 replaced with D; V176 replaced with A, G, I, L, S, T, or M; Q177 replaced with N; V178 replaced with A, G, I, L, S, T, or M; S179 replaced with A, G, I, L, T, M, or V; N180 replaced with Q; T182 replaced with A, G, I, L, S, M, or V; S183 replaced with A, G, I, L, T, M, or V; W184 replaced with F, or Y; D185 replaced with E; D186 replaced with E; I187 replaced with A, G, L, S, T, M, or V; Q188 replaced with N; V190 replaced with A, G, I, L, S, T, or M; E191 replaced with D; E192 replaced with D; F193 replaced with W, or Y; G194 replaced with A, I, L, S, T, M, or V; A195 replaced with G, I, L, S, T, M, or V; N196 replaced with Q; A197 replaced with G, I, L, S, T, M, or V; T198 replaced with A, G, I, L, S, M, or V; V199 replaced with A, G, I, L, S, T, or M; E200 replaced with D; T201 replaced with A, G, I, L, S, M, or V; A₂O₃ replaced with G, I, L, S, T, M, or V; A204 replaced with G, I, L, S, T, M, or V; E205 replaced with D; E206 replaced with D; T207 replaced with A, G, I, L, S, M, or V; M208 replaced with A, G, I, L, S, T, or V; N209 replaced with Q; T210 replaced with A, G, I, L, S, M, or V; S211 replaced with A, G, I, L, T, M, or V; G213 replaced with A, I, L, S, T, M, or V; T214 replaced with A, G, I, L, S, M, or V; A216 replaced with G, I, L, S, T, M, or V; A218 replaced with G, I, L, S, T, M, or V; A219 replaced with G, I, L, S, T, M, or V; E220 replaced with D; E221 replaced with D; T222 replaced with A, G, I, L, S, M, or V; M223 replaced with A, G, I, L, S, T, or V; N224 replaced with Q; T225 replaced with A, G, I, L, S, M, or V; S226 replaced with A, G, I, L, T, M, or V; G228 replaced with A, I, L, S, T, M, or V; T229 replaced with A, G, I, L, S, M, or V; A231 replaced with G, I, L, S, T, M, or V; A233 replaced with G, I, L, S, T, M, or V; A234 replaced with G, I, L, S, T, M, or V; E235 replaced with D; E236 replaced with D; T237 replaced with A, G, I, L, S, M, or V; M238 replaced with A, G, I, L, S, T, or V; T239 replaced with A, G, I, L, S, M, or V; T240 replaced with A, G, I, L, S, M, or V; S241 replaced with A, G, I, L, T, M, or V; G243 replaced with A, I, L, S, T, M, or V; T244 replaced with A, G, I, L, S, M, or V; A246 replaced with G, I, L, S, T, M, or V; A248 replaced with G, I, L, S, T, M, or V; A249 replaced with G, L, S, T, M, or V; E250 replaced with D; E251 replaced with D; T252 replaced with A, G, I, L, S, M, or V; M253 replaced with A, G, I, L, S, T, or V; T254 replaced with A, G, I, L, S, M, or V; T255 replaced with A, G, I, L, S, M, or V; S256 replaced with A, G, I, L, T, M, or V; G258 replaced with A, I, L, S, T, M, or V; T259 replaced with A, G, I, L, S, M, or V; A261 replaced with G, I, L, S, T, M, or V; A263 replaced with G, I, L, S, T, M, or V; A264 replaced with G, I, L, S, T, M, or V; E265 replaced with D; E266 replaced with D; T267 replaced with A, G, I, L, S, M, or V; M268 replaced with A, G, I, L, S, T, or V; T269 replaced with A, G, I, L, S, M, or V; T270 replaced with A, G, I, L, S, M, or V; S271 replaced with A, G, I, L, T, M, or V; G273 replaced with A, I, L, S, T, M, or V; T274 replaced with A, G, I, L, S, M, or V; A276 replaced with G, I, L, S, T, M, or V; S277 replaced with A, G, I, L, T, M, or V; S278 replaced with A, G, I, L, T, M, or V; H279 replaced with K, or R; Y280 replaced with F, or W; L281 replaced with A, G, I, S, T, M, or V; S282 replaced with A, G, I, L, T, M, or V; T284 replaced with A, G, I, L, S, M, or V; I285 replaced with A, G, L, S, T, M, or V; V286 replaced with A, G, I, L, S, T, or M; G287 replaced with A, I, L, S, T, M, or V; I288 replaced with A, G, L, S, T, M, or V; I289 replaced with A, G, L, S, T, M, or V; V290 replaced with A, G, I, L, S, T, or M; L291 replaced with A, G, I, S, T, M, or V; I292 replaced with A, G, L, S, T, M, or V; V293 replaced with A, G, I, L, S, T, or M; L294 replaced with A, G, I, S, T, M, or V; L295 replaced with A, G, I, S, T, M, or V; I296 replaced with A, G, L, S, T, M, or V; V297 replaced with A, G, I, L, S, T, or M; and/or F298 replaced with W, or Y; V299 replaced with A, G, I, L, S, T, or M.

In specific embodiments, the antibodies of the invention bind TR5 polypeptides or fragments or variants thereof (especially a fragment comprising or alternatively consisting of, the extracellular soluble domain of TR5), that contains any one or more of the following non-conservative mutations in TR5: M1 replaced with D, E, H, K, R, N, Q, F, W, Y, P, or C; Q2 replaced with D, E, H, K, R, A, G, I, L, S, T, M, V, F, W, Y, P, or C; G3 replaced with D, E, H, K, R, N, Q, F, W, Y, P, or C; V4 replaced with D, E, H, K, R, N, Q, F, W, Y, P, or C; K5 replaced with D, E, A, G, I, L, S, T, M, V, N, Q, F, W, Y, P, or C; E6 replaced with H, K, R, A, G, I, L, S, T, M, V, N, Q, F, W, Y, P, or C; R7 replaced with D, E, A, G, I, L, S, T, M, V, N, Q, F, W, Y, P, or C; F8 replaced with D, E, H, K, R, N, Q, A, G, I, L, S, T, M, V, P, or C; L9 replaced with D, E, H, K, R, N, Q, F, W, Y, P, or C; P10 replaced with D, E, H, K, R, A, G, I, L, S, T, M, V, N, Q, F, W, Y, or C; L11 replaced with D, E, H, K, R, N, Q, F, W, Y, P, or C; G12 replaced with D, E, H, K, R, N, Q, F, W, Y, P, or C; N13 replaced with D, E, H, K, R, A, G, I, L, S, T, M, V, F, W, Y, P, or C; S14 replaced with D, E, H, K, R, N, Q, F, W, Y, P, or C; G15 replaced with D, E, H, K, R, N, Q, F, W, Y, P, or C; D16 replaced with H, K, R, A, G, I, L, S, T, M, V, N, Q, F, W, Y, P, or C; R17 replaced with D, E, A, G, I, L, S, T, M, V, N, Q, F, W, Y, P, or C; A18 replaced with D, E, H, K, R, N, Q, F, W, Y, P, or C; P19 replaced with D, E, H, K, R, A, G, I, L, S, T, M, V, N, Q, F, W, Y, or C; R20 replaced with D, E, A, G, I, L, S, T, M, V, N, Q, F, W, Y, P, or C; P21 replaced with D, E, H, K, R, A, G, I, L, S, T, M, V, N, Q, F, W, Y, or C; P22 replaced with D, E, H, K, R, A, G, I, L, S, T, M, V, N, Q, F, W, Y, or C; D23 replaced with H, K, R, A, G, I, L, S, T, M, V, N, Q, F, W, Y, P, or C; G24 replaced with D, E, H, K, R, N, Q, F, W, Y, P, or C; R25 replaced with D, E, A, G, I, L, S, T, M, V, N, Q, F, W, Y, P, or C; G26 replaced with D, E, H, K, R, N, Q, F, W, Y, P, or C; R27 replaced with D, E, A, G, I, L, S, T, M, V, N, Q, F, W, Y, P, or C; V28 replaced with D, E, H, K, R, N, Q, F, W, Y, P, or C; R29 replaced with D, E, A, G, I, L, S, T, M, V, N, Q, F, W, Y, P, or C; P30 replaced with D, E, H, K, R, A, G, I, L, S, T, M, V, N, Q, F, W, Y, or C; R31 replaced with D, E, A, G, I, L, S, T, M, V, N, Q, F, W, Y, P, or C; T32 replaced with D, E, H, K, R, N, Q, F, W, Y, P, or C; Q33 replaced with D, E, H, K, R, A, G, I, L, S, T, M, V, F, W, Y, P, or C; D34 replaced with H, K, R, A, G, I, L, S, T, M, V, N, Q, F, W, Y, P, or C; G35 replaced with D, E, H, K, R, N, Q, F, W, Y, P, or C; V36 replaced with D, E, H, K, R, N, Q, F, W, Y, P, or C; G37 replaced with D, E, H, K, R, N, Q, F, W, Y, P, or C; N38 replaced with D, E, H, K, R, A, G, I, L, S, T, M, V, F, W, Y, P, or C; H39 replaced with D, E, A, G, I, L, S, T, M, V, N, Q, F, W, Y, P, or C; T40 replaced with D, E, H, K, R, N, Q, F, W, Y, P, or C; M41 replaced with D, E, H, K, R, N, Q, F, W, Y, P, or C; A42 replaced with D, E, H, K, R, N, Q, F, W, Y, P, or C; R43 replaced with D, E, A, G, I, L, S, T, M, V, N, Q, F, W, Y, P, or C; I44 replaced with D, E, H, K, R, N, Q, F, W, Y, P, or C; P45 replaced with D, E, H, K, R, A, G, I, L, S, T, M, V, N, Q, F, W, Y, or C; K46 replaced with D, E, A, G, I, L, S, T, M, V, N, Q, F, W, Y, P, or C; T47 replaced with D, E, H, K, R, N, Q, F, W, Y, P, or C; L48 replaced with D, E, H, K, R, N, Q, F, W, Y, P, or C; K49 replaced with D, E, A, G, I, L, S, T, M, V, N, Q, F, W, Y, P, or C; F50 replaced with D, E, H, K, R, N, Q, A, G, I, L, S, T, M, V, P, or C; V51 replaced with D, E, H, K, R, N, Q, F, W, Y, P, or C; V52 replaced with D, E, H, K, R, N, Q, F, W, Y, P, or C; V53 replaced with D, E, H, K, R, N, Q, F, W, Y, P, or C; I54 replaced with D, E, H, K, R, N, Q, F, W, Y, P, or C; V55 replaced with D, E, H, K, R, N, Q, F, W, Y, P, or C; A56 replaced with D, E, H, K, R, N, Q, F, W, Y, P, or C; V57 replaced with D, E, H, K, R, N, Q, F, W, Y, P, or C; L58 replaced with D, E, H, K, R, N, Q, F, W, Y, P, or C; L59 replaced with D, E, H, K, R, N, Q, F, W, Y, P, or C; P60 replaced with D, E, H, K, R, A, G, I, L, S, T, M, V, N, Q, F, W, Y, or C; V61 replaced with D, E, H, K, R, N, Q, F, W, Y, P, or C; L62 replaced with D, E, H, K, R, N, Q, F, W, Y, P, or C; A63 replaced with D, E, H, K, R, N, Q, F, W, Y, P, or C; Y64 replaced with D, E, H, K, R, N, Q, A, G, I, L, S, T, M, V, P, or C; S65 replaced with D, E, H, K, R, N, Q, F, W, Y, P, or C; A66 replaced with D, E, H, K, R, N, Q, F, W, Y, P, or C; T67 replaced with D, E, H, K, R, N, Q, F, W, Y, P, or C; T68 replaced with D, E, H, K, R, N, Q, F, W, Y, P, or C; A69 replaced with D, E, H, K, R, N, Q, F, W, Y, P, or C; R70 replaced with D, E, A, G, I, L, S, T, M, V, N, Q, F, W, Y, P, or C; Q71 replaced with D, E, H, K, R, A, G, I, L, S, T, M, V, F, W, Y, P, or C; E72 replaced with H, K, R, A, G, I, L, S, T, M, V, N, Q, F, W, Y, P, or C; E73 replaced with H, K, R, A, G, I, L, S, T, M, V, N, Q, F, W, Y, P, or C; V74 replaced with D, E, H, K, R, N, Q, F, W, Y, P, or C; P75 replaced with D, E, H, K, R, A, G, I, L, S, T, M, V, N, Q, F, W, Y, or C; Q76 replaced with D, E, H, K, R, A, G, I, L, S, T, M, V, F, W, Y, P, or C; Q77 replaced with D, E, H, K, R, A, G, I, L, S, T, M, V, F, W, Y, P, or C; T78 replaced with D, E, H, K, R, N, Q, F, W, Y, P, or C; V79 replaced with D, E, H, K, R, N, Q, F, W, Y, P, or C; A80 replaced with D, E, H, K, R, N, Q, F, W, Y, P, or C; P81 replaced with D, E, H, K, R, A, G, I, L, S, T, M, V, N, Q, F, W, Y, or C; Q82 replaced with D, E, H, K, R, A, G, I, L, S, T, M, V, F, W, Y, P, or C; Q83 replaced with D, E, H, K, R, A, G, I, L, S, T, M, V, F, W, Y, P, or C; Q84 replaced with D, E, H, K, R, A, G, I, L, S, T, M, V, F, W, Y, P, or C; R85 replaced with D, E, A, G, I, L, S, T, M, V, N, Q, F, W, Y, P, or C; H86 replaced with D, E, A, G, I, L, S, T, M, V, N, Q, F, W, Y, P, or C; S87 replaced with D, E, H, K, R, N, Q, F, W, Y, P, or C; F88 replaced with D, E, H, K, R, N, Q, A, G, I, L, S, T, M, V, P, or C; K89 replaced with D, E, A, G, I, L, S, T, M, V, N, Q, F, W, Y, P, or C; G90 replaced with D, E, H, K, R, N, Q, F, W, Y, P, or C; E91 replaced with H, K, R, A, G, I, L, S, T, M, V, N, Q, F, W, Y, P, or C; E92 replaced with H, K, R, A, G, I, L, S, T, M, V, N, Q, F, W, Y, P, or C; C93 replaced with D, E, H, K, R, A, G, I, L, S, T, M, V, N, Q, F, W, Y, or P; P94 replaced with D, E, H, K, R, A, G, I, L, S, T, M, V, N, Q, F, W, Y, or C; A95 replaced with D, E, H, K, R, N, Q, F, W, Y, P, or C; G96 replaced with D, E, H, K, R, N, Q, F, W, Y, P, or C; S97 replaced with D, E, H, K, R, N, Q, F, W, Y, P, or C; H98 replaced with D, E, A, G, I, L, S, T, M, V, N, Q, F, W, Y, P, or C; R99 replaced with D, E, A, G, I, L, S, T, M, V, N, Q, F, W, Y, P, or C; S100 replaced with D, E, H, K, R, N, Q, F, W, Y, P, or C; E101 replaced with H, K, R, A, G, I, L, S, T, M, V, N, Q, F, W, Y, P, or C; H102 replaced with D, E, A, G, I, L, S, T, M, V, N, Q, F, W, Y, P, or C; T103 replaced with D, E, H, K, R, N, Q, F, W, Y, P, or C; G104 replaced with D, E, H, K, R, N, Q, F, W, Y, P, or C; A105 replaced with D, E, H, K, R, N, Q, F, W, Y, P, or C; C106 replaced with D, E, H, K, R, A, G, I, L, S, T, M, V, N, Q, F, W, Y, or P; N107 replaced with D, E, H, K, R, A, G, I, L, S, T, M, V, F, W, Y, P, or C; P108 replaced with D, E, H, K, R, A, G, I, L, S, T, M, V, N, Q, F, W, Y, or C; C109 replaced with D, E, H, K, R, A, G, I, L, S, T, M, V, N, Q, F, W, Y, or P; T110 replaced with D, E, H, K, R, N, Q, F, W, Y, P, or C; E111 replaced with H, K, R, A, G, I, L, S, T, M, V, N, Q, F, W, Y, P, or C; G112 replaced with D, E, H, K, R, N, Q, F, W, Y, P, or C; V113 replaced with D, E, H, K, R, N, Q, F, W, Y, P, or C; D14 replaced with H, K, R, A, G, I, L, S, T, M, V, N, Q, F, W, Y, P, or C; Y115 replaced with D, E, H, K, R, N, Q, A, G, I, L, S, T, M, V, P, or C; T116 replaced with D, E, H, K, R, N, Q, F, W, Y, P, or C; N117 replaced with D, E, H, K, R, A, G, I, L, S, T, M, V, F, W, Y, P, or C; A18 replaced with D, E, H, K, R, N, Q, F, W, Y, P, or C; S119 replaced with D, E, H, K, R, N, Q, F, W, Y, P, or C; N120 replaced with D, E, H, K, R, A, G, I, L, S, T, M, V, F, W, Y, P, or C; N121 replaced with D, E, H, K, R, A, G, I, L, S, T, M, V, F, W, Y, P, or C; E122 replaced with H, K, R, A, G, I, L, S, T, M, V, N, Q, F, W, Y, P, or C; P123 replaced with D, E, H, K, R, A, G, I, L, S, T, M, V, N, Q, F, W, Y, or C; S124 replaced with D, E, H, K, R, N, Q, F, W, Y, P, or C; C125 replaced with D, E, H, K, R, A, G, I, L, S, T, M, V, N, Q, F, W, Y, or P; F126 replaced with D, E, H, K, R, N, Q, A, G, I, L, S, T, M, V, P, or C; P127 replaced with D, E, H, K, R, A, G, I, L, S, T, M, V, N, Q, F, W, Y, or C; C128 replaced with D, E, H, K, R, A, G, I, L, S, T, M, V, N, Q, F, W, Y, or P; T129 replaced with D, E, H, K, R, N, Q, F, W, Y, P, or C; V130 replaced with D, E, H, K, R, N, Q, F, W, Y, P, or C; C131 replaced with D, E, H, K, R, A, G, I, L, S, T, M, V, N, Q, F, W, Y, or P; K132 replaced with D, E, A, G, I, L, S, T, M, V, N, Q, F, W, Y, P, or C; S133 replaced with D, E, H, K, R, N, Q, F, W, Y, P, or C; D134 replaced with H, K, R, A, G, I, L, S, T, M, V, N, Q, F, W, Y, P, or C; Q135 replaced with D, E, H, K, R, A, G, I, L, S, T, M, V, F, W, Y, P, or C; K136 replaced with D, E, A, G, I, L, S, T, M, V, N, Q, F, W, Y, P, or C; H137 replaced with D, E, A, G, I, L, S, T, M, V, N, Q, F, W, Y, P, or C; K138 replaced with D, E, A, G, I, L, S, T, M, V, N, Q, F, W, Y, P, or C; S139 replaced with D, E, H, K, R, N, Q, F, W, Y, P, or C; S140 replaced with D, E, H, K, R, N, Q, F, W, Y, P, or C; C141 replaced with D, E, H, K, R, A, G, I, L, S, T, M, V, N, Q, F, W, Y, or P; T142 replaced with D, E, H, K, R, N, Q, F, W, Y, P, or C; M143 replaced with D, E, H, K, R, N, Q, F, W, Y, P, or C; T144 replaced with D, E, H, K, R, N, Q, F, W, Y, P, or C; R145 replaced with D, E, A, G, I, L, S, T, V, N, Q, F, W, Y, P, or C; D146 replaced with H, K, R, A, G, I, L, S, T, M, V, N, Q, F, W, Y, P, or C; T147 replaced with D, E, H, K, R, N, Q, F, W, Y, P, or C; V148 replaced with D, E, H, K, R, N, Q, F, W, Y, P, or C; C149 replaced with D, E, H, K, P, A, G, I, L, S, T, M, V, N, Q, F, W, Y, or P; Q150 replaced with D, E, H, K, R, A, G, I, L, S, T, M, V, F, W, Y, P, or C; C151 replaced with D, E, H, K, R, A, G, I, L, S, T, M, V, N, Q, F, W, Y, or P; K152 replaced with D, E, A, G, I, L, S, T, M, V, N, Q, F, W, Y, P, or C; E153 replaced with H, K, R, A, G, I, L, S, T, M, V, N, Q, F, W, Y, P, or C; G154 replaced with D, E, H, K, R, N, Q, F, W, Y, P, or C; T155 replaced with D, E, H, K, R, N, Q, F, W, Y, P, or C; F156 replaced with D, E, H, K, R, N, Q, A, G, I, L, S, T, M, V, P, or C; R157 replaced with D, E, A, G, I, L, S, T, M, V, N, Q, F, W, Y, P, or C; N158 replaced with D, E, H, K, R, A, G, I, L, S, T, M, V, F, W, Y, P, or C; E159 replaced with H, K, R, A, G, I, L, S, T, M, V, N, Q, F, W, Y, P, or C; N160 replaced with D, E, H, K, R, A, G, I, L, S, T, M, V, F, W, Y, P, or C; S161 replaced with D, E, H, K, R, N, Q, F, W, Y, P, or C; P162 replaced with D, E, H, K, R, A, G, I, L, S, T, M, V, N, Q, F, W, Y, or C; E163 replaced with H, K, R, A, G, I, L, S, T, M, V, N, Q, F, W, Y, P, or C; M164 replaced with D, E, H, K, R, N, Q, F, W, Y, P, or C; C165 replaced with D, E, H, K, R, A, G, I, L, S, T, M, V, N, Q, F, W, Y, or P; R166 replaced with D, E, A, G, I, L, S, T, M, V, N, Q, F, W, Y, P, or C; K167 replaced with D, E, A, G, I, L, S, T, M, V, N, Q, F, W, Y, P, or C; C168 replaced with D, E, H, K, R, A, G, I, L, S, T, M, V, N, Q, F, W, Y, or P; S169 replaced with D, E, H, K, R, N, Q, F, W, Y, P, or C; R170 replaced with D, E, A, G, I, L, S, T, M, V, N, Q, F, W, Y, P, or C; C171 replaced with D, E, H, K, R, A, G, I, L, S, T, M, V, N, Q, F, W, Y, or P; P172 replaced with D, E, H, K, R, A, G, I, L, S, T, M, V, N, Q, F, W, Y, or C; S173 replaced with D, E, H, K, R, N, Q, F, W, Y, P, or C; G174 replaced with D, E, H, K, R, N, Q, F, W, Y, P, or C; E175 replaced with H, K, R, A, G, I, L, S, T, M, V, N, Q, F, W, Y, P, or C; V176 replaced with D, E, H, K, R, N, Q, F, W, Y, P, or C; Q177 replaced with D, E, H, K, R, A, G, I, L, S, T, M, V, F, W, Y, P, or C; V178 replaced with D, E, H, K, R, N, Q, F, W, Y, P, or C; S179 replaced with D, E, H, K, R, N, Q, F, W, Y, P, or C; N180 replaced with D, E, H, K, R, A, G, I, L, S, T, M, V, F, W, Y, P, or C; C181 replaced with D, E, H, K, R, A, G, I, L, S, T, M, V, N, Q, F, W, Y, or P; T182 replaced with D, E, H, K, R, N, Q, F, W, Y, P, or C; S183 replaced with D, E, H, K, R, N, Q, F, W, Y, P, or C; W184 replaced with D, E, H, K, R, N, Q, A, G, I, L, S, T, M, V, P, or C; D185 replaced with H, K, R, A, G, I, L, S, T, M, V, N, Q, F, W, Y, P, or C; D186 replaced with H, K, R, A, G, I, L, S, T, M, V, N, Q, F, W, Y, P, or C; I187 replaced with D, E, H, K, R, N, Q, F, W, Y, P, or C; Q188 replaced with D, E, H, K, A, G, I, L, S, T, M, V, F, W, Y, P, or C; C189 replaced with D, E, H, K, R, A, G, I, L, S, T, M, V, N, Q, F, W, Y, or P; V190 replaced with D, E, H, K, R, N, Q, F, W, Y, P, or C; E191 replaced with H, K, R, A, G, I, L, S, T, M, V, N, Q, F, W, Y, P, or C; E192 replaced with H, K, R, A, G, I, L, S, T, M, V, N, Q, F, W, Y, P, or C; F193 replaced with D, E, H, K, R, N, Q, A, G, I, L, S, T, M, V, P, or C; G194 replaced with D, E, H, K, R, N, Q, F, W, Y, P, or C; A195 replaced with D, E, H, K, R, N, Q, F, W, Y, P, or C; N196 replaced with D, E, H, K, R, A, G, I, L, S, T, M, V, F, W, Y, P, or C; A197 replaced with D, E, H, K, R, N, Q, F, W, Y, P, or C; T198 replaced with D, E, H, K, R, N, Q, F, W, Y, P, or C; V199 replaced with D, E, H, K, R, N, Q, F, W, Y, P, or C; E200 replaced with H, K, R, A, G, I, L, S, T, M, V, N, Q, F, W, Y, P, or C; T201 replaced with D, E, H, K, R, N, Q, F, W, Y, P, or C; P202 replaced with D, E, H, K, R, A, G, I, L, S, T, M, V, N, Q, F, W, Y, or C; A₂O₃ replaced with D, E, H, K, R, N, Q, F, W, Y, P, or C; A204 replaced with D, E, H, K, R, N, Q, F, W, Y, P, or C; E205 replaced with H, K, R, A, G, I, L, S, T, M, V, N, Q, F, W, Y, P, or C; E206 replaced with H, K, R, A, G, I, L, S, T, M, V, N, Q, F, W, Y, P, or C; T207 replaced with D, E, H, K, R, N, Q, F, W, Y, P, or C; M208 replaced with D, E, H, K, R, N, Q, F, W, Y, P, or C; N209 replaced with D, E, H, K, R, A, G, I, L, S, T, M, V, F, W, Y, P, or C; T210 replaced with D, E, H, K, R, N, Q, F, W, Y, P, or C; S211 replaced with D, E, H, K, R, N, Q, F, W, Y, P, or C; P212 replaced with D, E, H, K, R, A, G, I, L, S, T, M, V, N, Q, F, W, Y, or C; G213 replaced with D, E, F, K, R, N, Q, F, W, Y, P, or C; T214 replaced with D, E, H, K, R, N, Q, F, W, Y, P, or C; P215 replaced with D, E, H, K, R, A, G, I, L, S, T, M, V, N, Q, F, W, Y, or C; A216 replaced with D, E, H, K, R, N, Q, F, W, Y, P, or C; P217 replaced with D, E, H, K, R, A, G, I, L, S, T, M, V, N, Q, F, W, Y, or C; A218 replaced with D, E, H, K, R, N, Q, F, W, Y, P, or C; A219 replaced with D, E, H, K, R, N, Q, F, W, Y, P, or C; E220 replaced with H, K, R, A, G, I, L, S, T, M, V, N, Q, F, W, Y, P, or C; E221 replaced with H, K, R, A, G, I, L, S, T, M, V, N, Q, F, W, Y, P, or C; T222 replaced with D, E, H, K, R, N, Q, F, W, Y, P, or C; M223 replaced with D, E, H, K, R, N, Q, F, W, Y, P, or C; N224 replaced with D, E, H, K, R, A, G, I, L, S, T, M, V, F, W, Y, P, or C; T225 replaced with D, E, H, K, R, N, Q, F, W, Y, P, or C; S226 replaced with D, E, H, K, R, N, Q, F, W, Y, P, or C; P227 replaced with D, E, H, K, R, A, G, I, L, S, T, M, V, N, Q, F, W, Y, or C; G228 replaced with D, E, H, K, R, N, Q, F, W, Y, P, or C; T229 replaced with D, E, H, K, R, N, Q, F, W, Y, P, or C; P230 replaced with D, E, H, K, R, A, G, I, L, S, T, M, V, N, Q, F, W, Y, or C; A231 replaced with D, E, H, K, R, N, Q, F, W, Y, P, or C; P232 replaced with D, E, H, K, R, A, G, I, L, S, T, M, V, N, Q, F, W, Y, or C; A233 replaced with D, E, H, K, R, N, Q, F, W, Y, P, or C; A234 replaced with D, E, H, K, R, N, Q, F, W, Y, P, or C; E235 replaced with H, K, R, A, G, I, L, S, T, M, V, N, Q, F, W, Y, P, or C; E236 replaced with H, K, R, A, G, I, L, S, T, M, V, N, Q, F, W, Y, P, or C; T237 replaced with D, E, H, K, R, N, Q, F, W, Y, P, or C; M238 replaced with D, E, H, K, R, N, Q, F, W, Y, P, or C; T239 replaced with D, E, H, K, R, N, Q, F, W, Y, P, or C; T240 replaced with D, E, H, K, R, N, Q, F, W, Y, P, or C; S241 replaced with D, E, H, K, R, N, Q, F, W, Y, P, or C; P242 replaced with D, E, H, K, R, A, G, I, L, S, T, V, N, Q, F, W, Y, or C; G243 replaced with D, E, H, K, R, N, Q, F, W, Y, P, or C; T244 replaced with D, E, H, K, R, N, Q, F, W, Y, P, or C; P245 replaced with D, E, H, K, R, A, G, I, L, S, T, M, V, N, Q, F, W, Y, or C; A246 replaced with D, E, H, K, R, N, Q, F, W, Y, P, or C; P247 replaced with D, E, H, K, R, A, G, I, L, S, T, M, V, N, Q, F, W, Y, or C; A248 replaced with D, E, H, K, R, N, Q, F, W, Y, P, or C; A249 replaced with D, E, H, K, R, N, Q, F, W, Y, P, or C; E250 replaced with H, K, R, A, G, I, L, S, T, M, V, N, Q, F, W, Y, P, or C; E251 replaced with H, K, R, A, G, I, L, S, T, M, V, N, Q, F, W, Y, P, or C; T252 replaced with D, E, H, K, R, N, Q, F, W, Y, P, or C; M253 replaced with D, E, H, K, R, N, Q, F, W, Y, P, or C; T254 replaced with D, E, H, K, R, N, Q, F, W, Y, P, or C; T255 replaced with D, E, H, K, R, N, Q, F, W, Y, P, or C; S256 replaced with D, E, H, K, R, N, Q, F, W, Y, P, or C; P257 replaced with D, E, H, K, R, A, G, I, L, S, T, M, V, N, Q, F, W, Y, or C; G258 replaced with D, E, H, K, R, N, Q, F, W, Y, P, or C; T259 replaced with D, E, H, K, R, N, Q, F, W, Y, P, or C; P260 replaced with D, E, H, K, R, A, G, I, L, S, T, M, V, N, Q, F, W, Y, or C; A261 replaced with D, E, H, K, R, N, Q, F, W, Y, P, or C; P262 replaced with D, E, H, K, R, A, G, I, L, S, T, M, V, N, Q, F, W, Y, or C; A263 replaced with D, E, H, K, R, N, Q, F, W, Y, P, or C; A264 replaced with D, E, H, K, R, N, Q, F, W, Y, P, or C; E265 replaced with H, K, R, A, G, I, L, S, T, M, V, N, Q, F, W, Y, P, or C; E266 replaced with H, K, R, A, G, I, L, S, T, M, V, N, Q, F, W, Y, P, or C; T267 replaced with D, E, H, K, R, N, Q, F, W, Y, P, or C; M268 replaced with D, E, H, K, R, N, Q, F, W, Y, P, or C; T269 replaced with D, E, H, K, R, N, Q, F, W, Y, P, or C; T270 replaced with D, E, H, K, R, N, Q, F, W, Y, P, or C; S271 replaced with D, E, H, K, R, N, Q, F, W, Y, P, or C; P272 replaced with D, E, H, K, R, A, G, I, L, S, T, M, V, N, Q, F, W, Y, or C; G273 replaced with D, E, H, K, R, N, Q, F, W, Y, P, or C; T274 replaced with D, E, H, K, R, N, Q, F, W, Y, P, or C; P275 replaced with D, E, H, K, R, A, G, I, L, S, T, M, V, N, Q, F, W, Y, or C; A276 replaced with D, E, H, K, R, N, Q, F, W, Y, P, or C; S277 replaced with D, E, H, K, R, N, Q, F, W, Y, P, or C; S278 replaced with D, E, H, K, R, N, Q, F, W, Y, P, or C; H279 replaced with D, E, A, G, I, L, S, T, M, V, N, Q, F, W, Y, P, or C; Y280 replaced with D, E, H, K, R, N, Q, A, G, I, L, S, T, M, V, P, or C; L281 replaced with D, E, H, K, R, N, Q, F, W, Y, P, or C; S282 replaced with D, E, H, K, R, N, Q, F, W, Y, P, or C; C283 replaced with D, E, H, K, R, A, G, I, L, S, T, M, V, N, Q, F, W, Y, or P; T284 replaced with D, E, H, K, R, N, Q, F, W, Y, P, or C; I285 replaced with D, E, H, K, R, N, Q, F, W, Y, P, or C; V286 replaced with D, E, H, K, R, N, Q, F, W, Y, P, or C; G287 replaced with D, E, H, K, R, N, Q, F, W, Y, P, or C; I288 replaced with D, E, H, K, R, N, Q, F, W, Y, P, or C; I289 replaced with D, E, H, K, R, N, Q, F, W, Y, P, or C; V290 replaced with D, E, H, K, R, N, Q, F, W, Y, P, or C; L291 replaced with D, E, H, K, R, N, Q, F, W, Y, P, or C; I292 replaced with D, E, H, K, R, N, Q, F, W, Y, P, or C; V293 replaced with D, E, H, K, R, N, Q, F, W, Y, P, or C; L294 replaced with D, E, H, K, R, N, Q, F, W, Y, P, or C; L295 replaced with D, E, H, K, R, N, Q, F, W, Y, P, or C; I296 replaced with D, E, H, K, R, N, Q, F, W, Y, P, or C; V297 replaced with D, E, H, K, R, N, Q, F, W, Y, P, or C; F298 replaced with D, E, H, K, R, N, Q, A, G, I, L, S, T, M, V, P, or C; and/or V299 replaced with D, E, H, K, R, N, Q, F, W, Y, P, or C.

Amino acids in the TR5 protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)). In preferred embodiments, antibodies of the present invention bind regions of TR5 that are essential for TR5 function. In other preferred embodiments, antibodies of the present invention bind regions of TR5 that are essential for. TR5 function and inhibit or abolish TR5 function. In other preferred embodiments, antibodies of the present invention bind regions of TR5 that are essential for TR5 function and enhance TR5 function.

Additionally, protein engineering may be employed to improve or alter the characteristics of TR5 polypeptides. Recombinant DNA technology known to those skilled in the art can be used to create novel mutant proteins or polypeptides including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Antibodies of the present invention may bind such modified TR5 polypeptides.

TR5 occurring variants that may be bound by the antibodies of the invention may be produced using art-known mutagenesis techniques, which include, but are not limited to oligonucleotide mediated mutagenesis, alanine scanning, PCR mutagenesis, site directed mutagenesis (see e.g., Carter et al., Nucl Acids Res. 13:4331 (1986); and Zoller et al., Nucl. Acids Res. 10:6487 (1982)), cassette mutagenesis (see e.g., Wells et al., Gene 34:315 (1985)), restriction selection mutagenesis (see e.g., Wells et al., Philos. Trans. R. Soc. London SerA 317:415 (1986)).

In preferred embodiments, the antibodies of the invention bind a 259 amino acid TR5 polypeptide (corresponding to amino acids residues 41-299 of SEQ ID NO:2) exhibiting two main structural domains. First, the extracellular TRAIL ligand binding domain was identified within residues from about 67 to about 280 in SEQ ID NO:2. Second, the transmembrane domain was identified within residues from about 281 to about 299 in SEQ ID NO:2. As mentioned above, however, TR5, lacks a putative intracellular signalling domain, thus, TR5 is also sometimes referred to as “TRID” (TRAIL Receptor Without an Intracellular Domain”).

The antibodies of the present invention also include antibodies that bind a polypeptide comprising, or alternatively, consisting of the polypeptide encoded by the deposited cDNA including the leader; the mature polypeptide encoded by the deposited the cDNA minus the leader (i.e., the mature protein); a polypeptide comprising amino acids about 1 to about 299 in SEQ ID NO:2; a polypeptide comprising amino acids about 41 to about 299 in SEQ ID NO:2; a polypeptide comprising amino acids about 42 to about 299 in SEQ ID NO:2 as well as polypeptides which are at least 80% identical, more preferably at least 90% or 95% identical, still more preferably at least 96%, 97%, 98%, or 99% identical to the polypeptides described above, and also include portions of such polypeptides with at least 30 amino acids and more preferably at least 50 amino acids.

By a polypeptide having an amino acid sequence at least, for example, 95% “identical” to a reference amino acid sequence of a TR5 polypeptide is intended that the amino acid sequence of the polypeptide is identical to the reference sequence except that the polypeptide sequence may include up to five amino acid alterations per each 100 amino acids of the reference amino acid of the TR5 polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in SEQ ID NO:2, or to the amino acid sequence encoded by the deposited cDNA clone, can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of this embodiment.

The polypeptide of the present invention have uses which include, but are not limited to, as sources for generating antibodies that bind the polypeptides of the invention, and as a molecular weight marker on SDS-PAGE gels or on molecular sieve gel filtration columns using methods well known to those of skill in the art.

The present application is also directed to proteins containing polypeptides at least 90%, 95%, 96%, 97%, 98% or 99% identical to the TR5 polypeptide sequence set forth herein as n¹-m¹. In preferred embodiments, the application is directed to proteins containing polypeptides at least 90%, 95%, 96%, 97%, 98% or 99% identical to polypeptides having the amino acid sequence of the specific TR5 N- and C-terminal deletions recited herein.

In certain preferred embodiments, TR5 proteins of the invention comprise fusion proteins as described above wherein the TR5 polypeptides are those described as n⁴-m³ herein. In preferred embodiments, the application is directed to nucleic acid molecules at least 90%, 95%, 96%, 97%, 98% or 99% identical to the nucleic acid sequences encoding polypeptides having the amino acid sequence of the specific N- and C-terminal deletions recited herein. Polynucleotides encoding these polypeptides are also encompassed by the invention.

TR7

In certain embodiments of the present invention, the antibodies of the present invention bind TR7 polypeptide, or fragments or variants thereof. The following section describes the TR7 polypeptides, fragments and variants that may be bound by the antibodies of the invention in more detail. The TR7 polypeptides, fragments and variants which may be bound by the antibodies of the invention are also described in, for example, International Publication Numbers WO98/41629, WO00/66156, and WO98/35986 which are herein incorporated by reference in their entireties.

In certain embodiments, the antibodies of the present invention immunospecifically bind TR7 polypeptide. An antibody that immunospecifically binds TR7 may, in some embodiments, bind fragments, variants (including species orthologs of TR7), multimers or modified forms of TR7. For example, an antibody immunospecific for TR7 may bind the TR7 moiety of a fusion protein comprising all or a portion of TR7.

TR7 proteins may be found as monomers or multimers (i.e., dimers, trimers, tetramers, and higher multimers). Accordingly, the present invention relates to antibodies that bind TR7 proteins found as monomers or as part of multimers. In specific embodiments, the TR7 polypeptides are monomers, dimers, trimers or tetramers. In additional embodiments, the multimers of the invention are at least dimers, at least trimers, or at least tetramers.

Antibodies of the invention may bind TR7 homomers or heteromers. As used herein, the term homomer, refers to a multimer containing only TR7 proteins of the invention (including TR7 fragments, variants, and fusion proteins, as described herein). These homomers may contain TR7 proteins having identical or different polypeptide sequences. In a specific embodiment, a homomer of the invention is a multimer containing only TR7 proteins having an identical polypeptide sequence. In another specific embodiment, antibodies of the invention bind TR7 homomers containing TR7 proteins having different polypeptide sequences. In specific embodiments, antibodies of the invention bind a TR7 homodimer (e.g., containing TR7 proteins having identical or different polypeptide sequences) or a homotrimer (e.g., containing TR7 proteins having identical or different polypeptide sequences). In additional embodiments, antibodies of the invention bind at least a homodimer, at least a homotrimer, or at least a homotetramer of TR7.

As used herein, the term heteromer refers to a multimer containing heterologous proteins (i.e., proteins containing polypeptide sequences that do not correspond to a polypeptide sequences encoded by the TR7 gene) in addition to the TR7 proteins of the invention. In a specific embodiment, antibodies of the invention bind a heterodimer, a heterotrimer, or a heterotetramer. In additional embodiments, the antibodies of the invention bind at least a homodimer, at least a homotrimer, or at least a homotetramer containing one or more TR7 polypeptides.

Multimers bound by one or more antibodies of the invention may be the result of hydrophobic, hydrophilic, ionic and/or covalent associations and/or may be indirectly linked, by for example, liposome formation. Thus, in one embodiment, multimers bound by one or more antibodies of the invention, such as, for example, homodimers or homotrimers, are formed when TR7 proteins contact one another in solution. In another embodiment, heteromultimers bound by one or more antibodies of the invention, such as, for example, heterotrimers or heterotetramers, are formed when TR7 proteins contact antibodies to the TR7 polypeptides (including antibodies to the heterologous polypeptide sequence in a fusion protein) in solution. In other embodiments, multimers bound by one or more antibodies of the invention are formed by covalent associations with and/or between the TR7 proteins of the invention. Such covalent associations may involve one or more amino acid residues contained in the polypeptide sequence of the protein (e.g., the polypeptide sequence recited in SEQ ID NO:3 or the polypeptide encoded by the deposited cDNA clone of ATCC Deposit 97920). In one instance, the covalent associations are cross-linking between cysteine residues located within the polypeptide sequences of the proteins which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a TR7 fusion protein. In one example, covalent associations are between the heterologous sequence contained in a fusion protein (see, e.g., U.S. Pat. No. 5,478,925). In a specific example, the covalent associations are between the heterologous sequence contained in a TR7-Fc fusion protein (as described herein). In another specific example, covalent associations of fusion proteins are between heterologous polypeptide sequences from another TNF family ligand/receptor member that is capable of forming covalently associated multimers, such as for example, osteoprotegerin (see, e.g., International Publication No. WO 98/49305, the contents of which are herein incorporated by reference in its entirety).

The multimers that may be bound by one or more antibodies of the invention may be generated using chemical techniques known in the art. For example, proteins desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers that may be bound by one or more antibodies of the invention may be generated using techniques known in the art to form one or more inter-molecule cross-links between the cysteine residues located within the polypeptide sequence of the proteins desired to be contained in the multimer (see, e.g., U.S. Pat. No. 5,478,925, which is herein incorporated by reference in its entirety). Further, proteins that may be bound by one or more antibodies of the invention may be routinely modified by the addition of cysteine or biotin to the C terminus or N-terminus of the polypeptide sequence of the protein and techniques known in the art may be applied to generate multimers containing one or more of these modified proteins (see, e.g., U.S. Pat. No. 5,478,925, which is herein incorporated by reference in its entirety). Additionally, techniques known in the art may be applied to generate liposomes containing the protein components desired to be contained in the multimer that may be bound by one or more antibodies of the invention (see, e.g., U.S. Pat. No. 5,478,925, which is herein incorporated by reference in its entirety).

Alternatively, multimers that may be bound by one or more antibodies of the invention may be generated using genetic engineering techniques known in the art. In one embodiment, proteins contained in multimers that may be bound by one or more antibodies of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer that may be bound by one or more antibodies of the invention are generated by ligating a polynucleotide sequence encoding a TR7 polypeptide to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant TR7 polypeptides which contain a transmembrane domain and which can be incorporated by membrane reconstitution techniques into liposomes (see, e.g., U.S. Pat. No. 5,478,925, which is herein incorporated by reference in its entirety). In another embodiment, two or more TR7 polypeptides are joined through synthetic linkers (e.g., peptide, carbohydrate or soluble polymer linkers). Examples include those peptide linkers described in U.S. Pat. No. 5,073,627 (hereby incorporated by reference). Proteins comprising multiple TR7 polypeptides separated by peptide linkers may be produced using conventional recombinant DNA technology. In specific embodiments, antibodies of the invention bind proteins comprising multiple TR7 polypeptides separated by peptide linkers.

Another method for preparing multimer TR7 polypeptides involves use of TR7 polypeptides fused to a leucine zipper or isoleucine polypeptide sequence. Leucine zipper domains and isoleucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric TR7 proteins are those described in PCT application WO 94/10308, hereby incorporated by reference. Recombinant fusion proteins comprising a soluble TR7 polypeptide fused to a peptide that dimerizes or trimerizes in solution are expressed in suitable host cells, and the resulting soluble multimeric TR7 is recovered from the culture supernatant using techniques known in the art. In specific embodiments, antibodies of the invention bind TR7-leucine zipper fusion protein monomers and/or TR7-leucine zipper fusion protein multimers.

Certain members of the TNF family of proteins are believed to exist in trimeric form (Beutler and Huffel, Science 264:667, 1994; Banner et al., Cell 73:431, 1993). Thus, trimeric TR7 may offer the advantage of enhanced biological activity. Preferred leucine zipper moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. In specific embodiments, antibodies of the invention bind TR7-leucine zipper fusion protein trimers.

Other peptides derived from naturally occurring trimeric proteins may be employed in preparing trimeric TR7. In specific embodiments, antibodies of the invention bind TR7-fusion protein monomers and/or TR7 fusion protein trimers.

The TR7 polypeptides are preferably provided in an isolated form, and preferably are substantially purified. By “isolated polypeptide” is intended a polypeptide removed from its native environment. Thus, a polypeptide produced and/or contained within a recombinant host cell is considered isolated for purposes of the present invention. Also, intended as an “isolated polypeptide” are polypeptides that have been purified, partially or substantially, from a recombinant host cell. For example, a recombinantly produced version of the TR7 polypeptide is substantially purified by the one-step method described in Smith and Johnson, Gene 67:3140 (1988).

Antibodies of the present invention may bind TR7 polypeptides or polypeptide fragments including polypeptides comprising or alternatively, consisting of, an amino acid sequence contained in SEQ ID NO:3, encoded by the cDNA contained in ATCC deposit Number 97920, or encoded by nucleic acids which hybridize (e.g., under stringent hybridization conditions) to the nucleotide sequence contained in the ATCC deposit Number 97920, or the complementary strand thereto. Protein fragments may be “free-standing,” or comprised within a larger polypeptide of which the fragment forms a part or region, most preferably as a single continuous region. Antibodies of the present invention may bind polypeptide fragments, including, for example, fragments that comprise or alternatively, consist of from about amino acid residues: 1 to 51, 52 to 78, 79 to 91, 92 to 111, 112 to 134, 135 to 151, 152 to 178, 179 to 180, 181 to 208, 209 to 218, 219 to 231, 232 to 251, 252 to 271, 272 to 291, 292 to 311, 312 to 323, 324 to 361, 362 to 391, 392 to 411 of SEQ ID NO:3. In this context “about” includes the particularly recited ranges, larger or smaller by several (5, 4, 3, 2, or 1) amino acids, at either extreme or at both extremes. Moreover, polypeptide fragments can be at least about 10, 20, 30, 40, 50, 60, 70, 80, 90, 100, 110, 120, 130, 140, or 150 amino acids in length. In this context “about” includes the particularly recited value, larger or smaller by several (5, 4, 3, 2, or 1) amino acids, at either extreme or at both extremes.

Preferred polypeptide fragments of the present invention include a member selected from the group: a polypeptide comprising or alternatively, consisting of, the TR7 receptor extracellular domain (predicted to constitute amino acid residues from about 52 to about 184 in SEQ ID NO:3); a polypeptide comprising or alternatively, consisting of, both TR7 cysteine rich domains (both of which may be found in the protein fragment consisting of amino acid residues from about 84 to about 179 in SEQ ID NO:3); a polypeptide comprising or alternatively, consisting of, the TR7 cysteine rich domain consisting of amino acid residues from about 84 to about 131 in SEQ ID NO:3); a polypeptide comprising or alternatively, consisting of, the TR7 cysteine rich domain consisting of amino acid residues from about 132 to about 179 in SEQ ID NO:3); a polypeptide comprising or alternatively, consisting of, the TR7 receptor transmembrane domain (predicted to constitute amino acid residues from about 185 to about 208 in SEQ ID NO:3); a polypeptide comprising or alternatively, consisting of, fragment of the predicted mature TR7 polypeptide, wherein the fragment has a TR7 functional activity (e.g., antigenic activity or biological activity); a polypeptide comprising or alternatively, consisting of, the TR7 receptor intracellular domain (predicted to constitute amino acid residues from about 209 to about 411 in SEQ ID NO:3); a polypeptide comprising or alternatively, consisting of, the TR7 receptor extracellular and intracellular domains with all or part of the transmembrane domain deleted; a polypeptide comprising, or alternatively consisting of, the TR7 receptor death domain (predicted to constitute amino acid residues from about 324 to about 391 in SEQ ID NO:3); and a polypeptide comprising, or alternatively, consisting of, one, two, three, four or more, epitope bearing portions of the TR7 receptor protein. In additional embodiments, the polypeptide fragments of the invention comprise, or alternatively, consist of, any combination of 1, 2, 3, 4, 5, 6, 7, or all 8 of the above members. As above, with the leader sequence, the amino acid residues constituting the TR7 receptor extracellular, transmembrane and intracellular domains have been predicted by computer analysis. Thus, as one of ordinary skill would appreciate, the amino acid residues constituting these domains may vary slightly (e.g., by about 1 to about 15 amino acid residues) depending on the criteria used to define each domain. Polypeptides encoded by these nucleic acid molecules are also encompassed by the invention.

As discussed above, it is believed that one or both of the extracellular cysteine rich motifs of TR7 is important for interactions between TR7 and its ligands (e.g., TRAIL). Accordingly, in highly preferred embodiments, antibodies of the present invention bind TR7 polypeptide fragments comprising, or alternatively consisting of, amino acid residues 84 to 131, and/or 132 to 179 of SEQ ID NO:3. In another highly preferred embodiment, antibodies of the present invention bind TR7 polypeptides comprising, or alternatively consisting of, both of the extracellular cysteine rich motifs (amino acid residues 84 to 179 of SEQ ID NO:3.) In another preferred embodiment, antibodies of the present invention bind TR7 polypeptides comprising, or alternatively consisting the extracellular soluble domain of TR7 (amino acid residues 52 to 184 of SEQ ID NO:2.) In other highly preferred embodiments, the antibodies of the invention that bind all or a portion of the extracellular soluble domain of TR7 (e.g., one or both cysteine rich domains) agonize the TR7 receptor.

In other highly preferred embodiments, the antibodies of the invention that bind all or a portion of the extracellular soluble domain of TR7 (e.g., one or both cysteine rich domains) induce cell death of the cell expressing the TR7 receptor.

Antibodies of the invention may also bind fragments comprising, or alternatively, consisting of structural or functional attributes of TR7. Such fragments include amino acid residues that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, surface forming regions, and high antigenic index regions (i.e., regions of polypeptides consisting of amino acid residues having an antigenic index of or equal to greater than 1.5, as identified using the default parameters of the Jameson-Wolf program) of TR7. Certain preferred regions are those disclosed in Table 5 and include, but are not limited to, regions of the aforementioned types identified by analysis of the amino acid sequence of SEQ ID NO:3, such preferred regions include; Garnier-Robson predicted alpha-regions, beta-regions, turn-regions, and coil-regions; Chou-Fasman predicted alpha-regions, beta-regions, and turn-regions; Kyte-Doolittle predicted hydrophilic regions and Hopp-Woods predicted hydrophobic regions; Eisenberg alpha and beta amphipathic regions; Emini surface-forming regions; and Jameson-Wolf high antigenic index regions, as predicted using the default parameters of these computer programs.

The data representing the structural or functional attributes of TR7 set forth in Table 5, as described above, was generated using the various modules and algorithms of the DNA*STAR set on default parameters. Column I represents the results of a Garnier-Robson analysis of alpha helical regions; Column II represents the results of a Chou-Fasman analysis of alpha helical regions; Column III represents the results of a Garnier Robson analysis of beta sheet regions; Column IV represents the results of a Chou-Fasman analysis of beta sheet regions; Column V represents the results of a Garnier Robson analysis of turn regions; Column VI represents the results of a Chou-Fasman analysis of turn regions; Column VII represents the results of a Garnier Robson analysis of coil regions; Column VIII represents a Kyte-Doolittle hydrophilicity plot; Column IX represents a Hopp-Woods hydrophobicity plot; Column X represents the results of an Eisenberg analysis of alpha amphipathic regions; Column XI represents the results of an Eisenberg analysis of beta amphipathic regions; Column XII represents the results of a Karplus-Schultz analysis of flexible regions; Column XIII represents the Jameson-Wolf antigenic index score; and Column XIV represents the Emini surface probability plot.

In a preferred embodiment, the data presented in columns VIII, IX, XIII, and XIV of Table 5 can be used to determine regions of TR7 which exhibit a high degree of potential for antigenicity. Regions of high antigenicity are determined from the data presented in columns VIII, IX XIII, and/or XIV by choosing values which represent regions of the polypeptide which are likely to be exposed on the surface of the polypeptide in an environment in which antigen recognition may occur in the process of initiation of an immune response. The columns in Table 5 present the result of different analyses of the TR7 protein sequence.

The above-mentioned preferred regions set out in Table 5 include, but are not limited to, regions of the aforementioned types identified by analysis of the amino acid sequence set out in SEQ ID NO:3. As set out in Table 5, such preferred regions include Garnier-Robson alpha-regions, beta-regions, turn-regions, and coil-regions, Chou-Fasman alpha-regions, beta-regions, and turn-regions, Kyte-Doolittle hydrophilic regions, Eisenberg alpha- and beta-amphipathic regions, Karplus-Schulz flexible regions, Jameson-Wolf regions of high antigenic index and Emini surface-forming regions. Preferably, antibodies of the present invention bind TR7 polypeptides or TR7 polypeptide fragments and variants comprising regions of TR7 that combine several structural features, such as several (e.g., 1, 2, 3, or 4) of the same or different region features set out above and in Table 5.

TABLE 5

| Res | Position | I | II | III | IV | V | VI | VII | VIII | IX | X | XI | XII | XIII | XIV |
|---|---|---|---|---|---|---|---|---|---|---|---|---|---|---|---|
| Met | 1 | A | . | . | . | . | . | . | 1.11 | −0.70 | . | * | . | 1.29 | 2.18 |
| Glu | 2 | A | . | . | . | . | . | . | 1.50 | −0.70 | . | * | . | 1.63 | 1.69 |
| Gln | 3 | A | . | . | . | . | T | . | 1.89 | −0.73 | . | * | . | 2.17 | 2.28 |
| Arg | 4 | . | . | . | . | T | T | . | 1.69 | −0.76 | . | * | . | 2.91 | 3.71 |
| Gly | 5 | . | . | . | . | T | T | . | 1.87 | −0.87 | . | * | F | 3.40 | 2.17 |
| Gln | 6 | . | . | . | . | T | T | . | 1.88 | −0.44 | . | * | F | 2.76 | 1.93 |
| Asn | 7 | . | . | . | . | . | . | C | 1.29 | −0.34 | . | * | F | 1.87 | 1.00 |
| Ala | 8 | . | . | . | . | . | . | C | 0.99 | 0.16 | . | . | F | 1.08 | 1.02 |
| Pro | 9 | . | . | . | . | . | . | C | 0.53 | 0.11 | . | * | . | 0.44 | 0.79 |
| Ala | 10 | A | . | . | . | . | . | . | 0.29 | 0.14 | . | * | . | −0.10 | 0.48 |
| Ala | 11 | A | . | . | . | . | T | . | 0.40 | 0.24 | . | . | . | 0.10 | 0.48 |
| Ser | 12 | A | . | . | . | . | T | . | 0.44 | −0.26 | . | * | F | 0.85 | 0.61 |
| Gly | 13 | A | . | . | . | . | T | . | 1.14 | −0.69 | . | * | F | 1.30 | 1.22 |
| Ala | 14 | A | . | . | . | . | T | . | 1.32 | −1.19 | . | * | F | 1.30 | 2.36 |
| Arg | 15 | A | . | . | . | T | . | . | 1.57 | −1.19 | . | * | F | 1.50 | 2.39 |
| Lys | 16 | . | . | . | . | T | . | . | 1.94 | −1.14 | . | . | F | 1.50 | 2.39 |
| Arg | 17 | . | . | . | . | T | . | . | 1.90 | −1.14 | . | * | F | 1.80 | 3.66 |
| His | 18 | . | . | . | . | . | . | C | 2.03 | −1.21 | * | * | F | 1.90 | 1.85 |
| Gly | 19 | . | . | . | . | . | T | C | 2.73 | −0.79 | * | * | F | 2.40 | 1.43 |
| Pro | 20 | . | . | . | . | . | T | C | 2.62 | −0.79 | * | * | F | 2.70 | 1.43 |
| Gly | 21 | . | . | . | . | . | T | C | 1.99 | −0.79 | * | . | F | 3.00 | 1.82 |
| Pro | 22 | . | . | . | . | . | T | C | 1.99 | −0.79 | . | * | F | 2.70 | 1.86 |
| Arg | 23 | . | A | . | . | . | . | C | 1.68 | −1.21 | * | . | F | 2.30 | 2.35 |
| Glu | 24 | . | A | B | . | . | . | . | 1.43 | −1.21 | * | . | F | 2.10 | 2.35 |
| Ala | 25 | . | A | . | . | T | . | . | 1.76 | −1.14 | * | . | F | 2.50 | 1.54 |
| Arg | 26 | . | A | . | . | T | . | . | 1.89 | −1.57 | * | . | F | 2.50 | 1.54 |
| Gly | 27 | . | . | . | . | T | . | . | 1.76 | −1.14 | * | . | F | 3.00 | 1.37 |
| Ala | 28 | . | . | . | . | T | . | C | 1.43 | −0.71 | * | * | F | 2.70 | 1.35 |
| Arg | 29 | . | . | . | . | . | T | C | 1.54 | −0.79 | * | * | F | 2.66 | 1.06 |
| Pro | 30 | . | . | . | . | . | T | C | 1.28 | −0.79 | * | * | F | 2.62 | 2.10 |
| Gly | 31 | . | . | . | . | . | T | C | 0.96 | −0.57 | * | * | F | 2.58 | 1.54 |
| Pro | 32 | . | . | . | . | . | T | C | 1.34 | −0.64 | * | * | F | 2.54 | 1.22 |
| Arg | 33 | . | . | . | . | . | . | C | 1.62 | −0.64 | * | * | F | 2.60 | 1.58 |
| Val | 34 | . | . | . | . | . | . | C | 0.70 | −0.59 | * | * | F | 2.34 | 2.30 |
| Pro | 35 | . | . | B | . | . | . | . | 0.06 | −0.33 | * | * | F | 1.58 | 1.23 |
| Lys | 36 | . | . | B | B | . | . | . | −0.41 | −0.11 | * | . | F | 0.97 | 0.46 |
| Thr | 37 | . | . | B | B | . | . | . | −1.06 | 0.57 | * | * | F | −0.19 | 0.52 |
| Leu | 38 | . | . | B | B | . | . | . | −2.02 | 0.57 | * | * | . | −0.60 | 0.25 |
| Val | 39 | . | . | B | B | . | . | . | −1.76 | 0.79 | . | . | . | −0.60 | 0.09 |
| Leu | 40 | A | . | . | B | . | . | . | −2.13 | 1.29 | . | . | . | −0.60 | 0.06 |
| Val | 41 | A | . | . | B | . | . | . | −3.03 | 1.30 | . | . | . | −0.60 | 0.08 |
| Val | 42 | A | . | . | B | . | . | . | −3.53 | 1.26 | . | . | . | −0.60 | 0.08 |
| Ala | 43 | A | . | . | B | . | . | . | −3.53 | 1.30 | . | . | . | −0.60 | 0.08 |
| Ala | 44 | A | . | . | B | . | . | . | −3.49 | 1.30 | . | . | . | −0.60 | 0.09 |
| Val | 45 | A | . | . | B | . | . | . | −3.53 | 1.34 | . | . | . | −0.60 | 0.10 |
| Leu | 46 | A | . | . | B | . | . | . | −2.98 | 1.34 | . | . | . | −0.60 | 0.07 |
| Leu | 47 | A | . | . | B | . | . | . | −2.71 | 1.23 | . | . | . | −0.60 | 0.09 |
| Leu | 48 | A | . | . | B | . | . | . | −2.12 | 1.23 | . | . | . | −0.60 | 0.13 |
| Val | 49 | A | . | . | B | . | . | . | −1.83 | 0.59 | . | . | . | −0.60 | 0.27 |
| Ser | 50 | A | . | . | B | . | . | . | −1.57 | 0.29 | . | * | . | −0.30 | 0.44 |
| Ala | 51 | A | A | . | . | . | . | . | −1.57 | 0.10 | . | . | . | −0.30 | 0.54 |
| Glu | 52 | A | A | . | . | . | . | . | −1.64 | 0.10 | . | . | . | −0.30 | 0.60 |
| Ser | 53 | A | A | . | B | . | . | . | −1.14 | 0.14 | . | . | . | −0.30 | 0.31 |
| Ala | 54 | A | A | . | B | . | . | . | −0.29 | 0.24 | . | . | . | −0.30 | 0.45 |
| Leu | 55 | A | A | . | B | . | . | . | 0.01 | 0.14 | . | . | . | −0.30 | 0.45 |
| Ile | 56 | A | A | . | B | . | . | . | 0.60 | 0.54 | . | . | . | −0.60 | 0.58 |
| Thr | 57 | A | A | . | B | . | . | . | −0.21 | 0.16 | . | . | F | −0.15 | 0.96 |
| Gln | 58 | A | A | . | B | . | . | . | −0.50 | 0.34 | . | . | F | −0.15 | 0.96 |
| Gln | 59 | A | A | . | B | . | . | . | −0.12 | 0.16 | . | . | F | 0.00 | 1.38 |
| Asp | 60 | . | A | . | B | T | . | . | 0.69 | −0.10 | . | . | F | 1.00 | 1.48 |
| Leu | 61 | . | A | . | . | . | . | C | 1.58 | −0.19 | . | * | F | 0.80 | 1.48 |
| Ala | 62 | . | A | . | . | . | . | C | 2.00 | −0.19 | . | * | F | 0.80 | 1.48 |
| Pro | 63 | . | A | . | . | . | . | C | 1.41 | −0.59 | . | * | F | 1.10 | 1.73 |
| Gln | 64 | . | A | . | . | T | . | . | 0.82 | −0.09 | . | * | F | 1.00 | 2.13 |
| Gln | 65 | A | A | . | . | . | . | . | 0.61 | −0.27 | . | * | F | 0.60 | 2.13 |
| Arg | 66 | A | A | . | . | . | . | . | 1.42 | −0.34 | . | * | F | 0.60 | 2.13 |
| Ala | 67 | A | A | . | . | . | . | . | 2.01 | −0.37 | . | * | F | 0.94 | 2.13 |
| Ala | 68 | A | A | . | . | . | . | . | 2.27 | −0.37 | * | * | F | 1.28 | 2.13 |
| Pro | 69 | A | A | . | . | . | . | . | 2.38 | −0.77 | * | * | F | 1.92 | 2.17 |
| Gln | 70 | . | A | . | . | T | . | . | 2.08 | −0.77 | * | . | F | 2.66 | 4.21 |
| Gln | 71 | . | . | . | . | T | T | . | 1.67 | −0.89 | * | * | F | 3.40 | 5.58 |
| Lys | 72 | . | . | . | . | T | T | . | 2.04 | −1.00 | . | . | F | 3.06 | 4.84 |
| Arg | 73 | . | . | . | . | T | T | . | 2.33 | −1.00 | . | . | F | 2.97 | 4.32 |
| Ser | 74 | . | . | . | . | . | T | C | 2.54 | −1.01 | . | . | F | 2.68 | 3.34 |
| Ser | 75 | . | . | . | . | . | T | C | 2.20 | −1.41 | . | . | F | 2.59 | 2.89 |
| Pro | 76 | . | . | . | . | T | T | . | 1.39 | −0.99 | . | . | F | 2.70 | 1.46 |
| Ser | 77 | . | . | . | . | T | T | . | 0.68 | −0.30 | . | . | F | 2.50 | 0.90 |
| Glu | 78 | . | . | . | . | T | T | . | 0.36 | −0.11 | . | * | F | 2.25 | 0.36 |
| Gly | 79 | . | . | . | . | T | . | . | 0.44 | −0.07 | . | . | F | 1.80 | 0.36 |
| Leu | 80 | . | . | . | . | T | . | . | 0.40 | −0.07 | . | . | F | 1.55 | 0.42 |
| Cys | 81 | . | . | . | . | . | . | C | 0.58 | −0.03 | . | . | . | 0.95 | 0.24 |
| Pro | 82 | . | . | . | . | . | T | C | 0.84 | 0.47 | * | . | F | 0.15 | 0.33 |
| Pro | 83 | . | . | . | . | T | T | . | −0.04 | 0.54 | * | . | F | 0.35 | 0.54 |
| Gly | 84 | . | . | . | . | T | T | . | 0.00 | 0.54 | * | . | . | 0.20 | 0.70 |
| His | 85 | . | . | . | . | . | T | C | 0.81 | 0.36 | * | . | . | 0.30 | 0.61 |
| His | 86 | . | . | . | . | . | . | C | 1.48 | −0.07 | * | . | . | 0.70 | 0.68 |
| Ile | 87 | . | . | . | . | . | . | C | 1.34 | −0.50 | * | * | . | 1.19 | 1.15 |
| Ser | 88 | . | . | . | . | . | . | C | 1.67 | −0.50 | * | * | F | 1.53 | 0.84 |
| Glu | 89 | . | . | . | . | T | . | . | 2.01 | −1.00 | * | * | F | 2.52 | 1.21 |
| Asp | 90 | . | . | . | . | T | . | . | 1.38 | −1.50 | * | * | F | 2.86 | 2.88 |
| Gly | 91 | . | . | . | . | T | T | . | 0.52 | −1.61 | * | * | F | 3.40 | 1.15 |
| Arg | 92 | . | . | . | . | T | T | . | 1.11 | −1.31 | * | * | F | 2.91 | 0.47 |
| Asp | 93 | . | . | . | . | T | T | . | 0.74 | −0.93 | . | * | F | 2.57 | 0.37 |
| Cys | 94 | . | . | . | . | T | T | . | 0.79 | −0.36 | . | * | . | 1.78 | 0.20 |
| Ile | 95 | . | . | . | . | T | . | . | 0.54 | −0.79 | . | * | . | 1.54 | 0.21 |
| Ser | 96 | . | . | . | . | T | . | . | 0.54 | −0.03 | . | * | . | 1.18 | 0.19 |
| Cys | 97 | . | . | . | . | T | T | . | 0.43 | 0.40 | . | * | . | 0.76 | 0.36 |
| Lys | 98 | . | . | . | . | T | T | . | 0.43 | 0.23 | . | . | . | 1.34 | 0.88 |
| Tyr | 99 | . | . | . | . | T | T | . | 0.86 | −0.46 | . | * | F | 2.52 | 1.10 |
| Gly | 100 | . | . | . | . | T | T | . | 1.44 | −0.09 | . | * | F | 2.80 | 3.22 |
| Gln | 101 | . | . | . | . | T | T | . | 1.43 | −0.27 | * | . | F | 2.52 | 2.16 |
| Asp | 102 | . | . | . | . | T | T | . | 2.07 | 0.21 | * | * | F | 1.64 | 1.99 |
| Tyr | 103 | . | . | . | . | T | T | . | 1.73 | −0.04 | * | * | F | 1.96 | 2.73 |
| Ser | 104 | . | . | . | . | T | T | . | 1.98 | 0.44 | * | . | F | 0.78 | 1.66 |
| Thr | 105 | . | . | . | . | T | . | . | 2.32 | 0.44 | * | . | F | 0.30 | 1.60 |
| His | 106 | . | . | . | . | T | . | . | 1.51 | 0.44 | * | . | . | 0.15 | 1.70 |
| Trp | 107 | . | . | . | . | T | T | . | 0.70 | 0.37 | * | . | . | 0.65 | 1.05 |
| Asn | 108 | . | . | . | . | T | T | . | 0.24 | 0.67 | . | . | . | 0.20 | 0.60 |
| Asp | 109 | . | . | . | . | T | T | . | −0.12 | 0.97 | * | . | . | 0.20 | 0.38 |
| Leu | 110 | A | . | . | . | . | T | . | −0.62 | 1.04 | * | * | . | −0.20 | 0.19 |
| Leu | 111 | . | . | . | B | T | . | . | −0.48 | 0.81 | * | * | . | −0.20 | 0.10 |
| Phe | 112 | . | . | . | B | T | . | . | −0.86 | 0.41 | * | * | . | −0.20 | 0.12 |
| Cys | 113 | . | . | . | B | T | . | . | −1.17 | 0.99 | * | * | . | −0.20 | 0.08 |
| Leu | 114 | . | . | . | B | T | . | . | −1.06 | 0.79 | . | * | . | −0.20 | 0.13 |
| Arg | 115 | . | . | . | B | T | . | . | −0.91 | 0.10 | . | * | . | 0.10 | 0.30 |
| Cys | 116 | . | . | . | B | T | . | . | −0.10 | −0.11 | . | . | . | 0.70 | 0.30 |
| Thr | 117 | . | . | . | B | T | . | . | 0.30 | −0.69 | . | * | . | 1.00 | 0.61 |
| Arg | 118 | . | . | . | B | T | . | . | 0.62 | −0.99 | . | . | F | 1.49 | 0.42 |
| Cys | 119 | . | . | . | . | T | T | . | 1.43 | −0.56 | * | . | F | 2.23 | 0.77 |
| Asp | 120 | . | . | . | . | T | T | . | 0.47 | −1.13 | * | . | F | 2.57 | 0.92 |
| Ser | 121 | . | . | . | . | T | T | . | 1.13 | −0.97 | . | * | F | 2.91 | 0.35 |
| Gly | 122 | . | . | . | . | T | T | . | 0.63 | −0.97 | . | * | F | 3.40 | 1.13 |
| Glu | 123 | . | A | . | . | T | . | . | 0.22 | −0.86 | . | * | F | 2.51 | 0.56 |
| Val | 124 | A | A | . | . | . | . | . | 0.68 | −0.47 | . | * | F | 1.47 | 0.56 |
| Glu | 125 | . | A | . | . | T | . | . | 0.01 | −0.43 | . | * | . | 1.38 | 0.87 |
| Leu | 126 | . | A | . | . | T | . | . | 0.00 | −0.29 | . | * | . | 1.04 | 0.27 |
| Ser | 127 | . | . | . | . | . | T | C | 0.03 | 0.20 | . | * | F | 0.45 | 0.52 |
| Pro | 128 | . | . | . | . | T | T | . | −0.28 | 0.04 | . | * | F | 0.93 | 0.44 |
| Cys | 129 | . | . | . | . | T | T | . | 0.69 | 0.53 | . | * | F | 0.91 | 0.77 |
| Thr | 130 | . | . | . | . | T | T | . | 0.69 | −0.16 | . | * | F | 2.24 | 1.12 |
| Thr | 131 | . | . | . | . | T | . | . | 1.19 | −0.14 | . | * | F | 2.32 | 1.16 |
| Thr | 132 | . | . | . | . | T | T | . | 0.63 | −0.09 | . | * | F | 2.80 | 3.13 |
| Arg | 133 | . | . | . | . | T | T | . | 0.18 | −0.01 | . | . | F | 2.52 | 1.61 |
| Asn | 134 | . | . | . | . | T | T | . | 0.84 | 0.07 | . | . | F | 1.49 | 0.60 |
| Thr | 135 | . | . | . | . | T | T | . | 0.49 | −0.01 | . | . | F | 1.81 | 0.72 |
| Val | 136 | . | . | . | . | T | . | C | 0.80 | 0.07 | * | . | . | 0.58 | 0.20 |
| Cys | 137 | . | A | . | . | T | . | . | 1.11 | 0.07 | * | . | . | 0.10 | 0.21 |
| Gln | 138 | . | A | B | . | . | . | . | 0.66 | −0.33 | * | . | . | 0.30 | 0.25 |
| Cys | 139 | . | A | . | . | T | . | . | 0.34 | −0.39 | . | . | . | 0.70 | 0.34 |
| Glu | 140 | A | A | . | . | . | . | . | −0.04 | −0.54 | * | * | F | 0.75 | 0.91 |
| Glu | 141 | A | A | . | . | . | . | . | 0.92 | −0.33 | * | * | F | 0.45 | 0.46 |
| Gly | 142 | . | A | . | . | T | . | . | 1.59 | −0.73 | . | * | F | 1.30 | 1.67 |
| Thr | 143 | A | A | . | . | . | . | . | 1.59 | −1.30 | . | * | F | 0.90 | 1.67 |
| Phe | 144 | A | A | . | . | . | . | . | 2.26 | −1.30 | . | * | F | 0.90 | 1.67 |
| Arg | 145 | A | A | . | . | . | . | . | 1.96 | −1.30 | . | * | F | 0.90 | 2.81 |
| Glu | 146 | A | A | . | . | . | . | . | 1.74 | −1.34 | . | * | F | 0.90 | 2.61 |
| Glu | 147 | A | A | . | . | . | . | . | 2.09 | −1.40 | . | * | F | 0.90 | 4.66 |
| Asp | 148 | A | A | . | . | . | . | . | 1.80 | −2.19 | . | * | F | 0.90 | 4.12 |
| Ser | 149 | A | . | . | . | . | T | . | 1.83 | −1.57 | . | * | F | 1.30 | 2.35 |
| Pro | 150 | A | . | . | . | . | T | . | 1.83 | −1.00 | . | . | F | 1.15 | 0.73 |
| Glu | 151 | A | . | . | . | . | T | . | 1.88 | −1.00 | * | . | F | 1.15 | 0.85 |
| Met | 152 | A | . | . | . | . | T | . | 1.21 | −1.00 | * | * | . | 1.49 | 1.28 |
| Cys | 153 | A | . | . | . | . | T | . | 1.32 | −0.81 | * | * | . | 1.68 | 0.44 |
| Arg | 154 | A | . | . | . | . | T | . | 1.31 | −1.24 | * | . | . | 2.02 | 0.50 |
| Lys | 155 | . | . | . | . | T | T | . | 1.18 | −0.76 | * | * | F | 2.91 | 0.73 |
| Cys | 156 | . | . | . | . | T | T | . | 0.51 | −0.94 | * | . | F | 3.40 | 1.35 |
| Arg | 157 | . | . | . | . | T | . | . | 0.90 | −0.94 | * | . | F | 2.71 | 0.37 |
| Thr | 158 | . | . | . | . | T | . | . | 1.68 | −0.51 | * | . | F | 2.37 | 0.28 |
| Gly | 159 | . | . | . | . | T | . | . | 1.22 | −0.51 | * | . | F | 2.43 | 1.04 |
| Cys | 160 | . | . | . | . | . | T | C | 0.58 | −0.66 | . | * | F | 2.19 | 0.53 |
| Pro | 161 | . | . | . | . | T | T | . | 0.39 | −0.04 | . | * | F | 2.00 | 0.36 |
| Arg | 162 | . | . | . | . | T | T | . | 0.32 | 0.11 | . | * | F | 1.65 | 0.27 |
| Gly | 163 | . | . | . | . | T | T | . | −0.22 | −0.31 | * | * | . | 2.50 | 1.01 |
| Met | 164 | . | . | B | B | . | . | . | −0.22 | −0.24 | * | * | . | 1.30 | 0.48 |
| Val | 165 | . | . | B | B | . | . | . | 0.44 | −0.24 | * | * | . | 1.30 | 0.24 |
| Lys | 166 | . | . | B | B | . | . | . | −0.01 | −0.24 | * | * | . | 1.30 | 0.41 |
| Val | 167 | . | . | B | . | . | T | . | −0.43 | −0.10 | * | * | F | 1.85 | 0.22 |
| Gly | 168 | . | . | . | . | T | T | . | −0.30 | −0.23 | . | . | F | 2.25 | 0.44 |
| Asp | 169 | . | . | . | . | T | T | . | 0.01 | −0.44 | . | . | F | 2.50 | 0.34 |
| Cys | 170 | . | . | . | . | T | T | . | 0.57 | 0.47 | . | * | F | 1.35 | 0.48 |
| Thr | 171 | . | . | . | . | . | T | C | 0.52 | 0.21 | . | * | F | 1.20 | 0.65 |
| Pro | 172 | . | . | . | . | T | T | . | 0.49 | −0.21 | . | * | F | 1.75 | 0.65 |
| Trp | 173 | . | . | . | . | T | T | . | 0.83 | 0.47 | . | * | F | 0.60 | 0.84 |
| Ser | 174 | A | . | . | . | . | T | . | 0.17 | −0.10 | . | * | F | 1.00 | 1.01 |
| Asp | 175 | A | A | . | . | . | . | . | −0.02 | −0.01 | . | . | F | 0.45 | 0.35 |
| Ile | 176 | A | A | . | . | . | . | . | 0.26 | 0.20 | * | * | . | −0.30 | 0.25 |
| Glu | 177 | A | A | . | . | . | . | . | 0.51 | −0.21 | * | . | . | 0.30 | 0.25 |
| Cys | 178 | A | A | . | . | . | . | . | 0.80 | −0.60 | * | . | . | 0.60 | 0.30 |
| Val | 179 | A | A | . | . | . | . | . | 0.80 | −0.60 | * | * | . | 0.60 | 0.74 |
| His | 180 | A | A | . | . | . | . | . | 0.46 | −0.90 | . | * | . | 0.60 | 0.58 |
| Lys | 181 | A | A | . | . | . | . | . | 0.46 | −0.47 | * | . | F | 0.60 | 1.06 |
| Glu | 182 | A | . | . | . | . | T | . | −0.43 | −0.36 | * | . | F | 1.00 | 1.00 |
| Ser | 183 | A | . | . | . | . | T | . | −0.66 | −0.31 | . | . | F | 0.85 | 0.52 |
| Gly | 184 | A | . | . | . | T | T | . | −0.14 | −0.13 | . | . | F | 1.25 | 0.18 |
| Ile | 185 | A | . | . | . | . | T | . | −0.97 | 0.30 | . | . | . | 0.10 | 0.10 |
| Ile | 186 | . | . | B | B | . | . | . | −1.32 | 0.94 | . | * | . | −0.60 | 0.06 |
| Ile | 187 | . | . | B | B | . | . | . | −2.18 | 1.04 | . | . | . | −0.60 | 0.08 |
| Gly | 188 | . | . | B | B | . | . | . | −2.47 | 1.26 | . | * | . | −0.60 | 0.09 |
| Val | 189 | . | . | B | B | . | . | . | −2.71 | 1.07 | . | . | . | −0.60 | 0.13 |
| Thr | 190 | A | . | . | B | . | . | . | −2.68 | 0.89 | . | * | . | −0.60 | 0.18 |
| Val | 191 | A | . | . | B | . | . | . | −2.64 | 0.84 | . | . | . | −0.60 | 0.14 |
| Ala | 192 | A | . | . | B | . | . | . | −2.57 | 1.06 | . | * | . | −0.60 | 0.14 |
| Ala | 193 | A | . | . | B | . | . | . | −3.11 | 1.10 | . | . | . | −0.60 | 0.08 |
| Val | 194 | A | . | . | B | . | . | . | −3.11 | 1.30 | . | . | . | −0.60 | 0.07 |
| Val | 195 | A | . | . | B | . | . | . | −3.39 | 1.30 | . | . | . | −0.60 | 0.05 |
| Leu | 196 | A | . | . | B | . | . | . | −3.39 | 1.30 | . | . | . | −0.60 | 0.05 |
| Ile | 197 | A | . | . | B | . | . | . | −3.50 | 1.44 | . | . | . | −0.60 | 0.05 |
| Val | 198 | A | . | . | B | . | . | . | −3.77 | 1.59 | . | . | . | −0.60 | 0.06 |
| Ala | 199 | A | . | . | B | . | . | . | −3.58 | 1.59 | . | . | . | −0.60 | 0.06 |
| Val | 200 | A | . | . | B | . | . | . | −2.68 | 1.47 | . | . | . | −0.60 | 0.04 |
| Phe | 201 | A | . | . | B | . | . | . | −2.17 | 0.79 | . | . | . | −0.60 | 0.12 |
| Val | 202 | A | . | . | B | . | . | . | −2.09 | 0.53 | . | . | . | −0.60 | 0.16 |
| Cys | 203 | A | . | . | . | . | T | . | −2.04 | 0.71 | . | . | . | −0.20 | 0.17 |
| Lys | 204 | A | . | . | . | . | T | . | −1.74 | 0.76 | . | . | . | −0.20 | 0.17 |
| Ser | 205 | A | . | . | . | . | T | . | −0.84 | 0.89 | . | . | . | −0.20 | 0.24 |
| Leu | 206 | A | . | . | . | . | T | . | −0.10 | 0.24 | . | . | . | 0.10 | 0.88 |
| Leu | 207 | A | A | . | . | . | . | . | −0.10 | −0.33 | . | . | . | 0.30 | 0.88 |
| Trp | 208 | A | A | . | . | . | . | . | −0.24 | 0.31 | . | . | . | −0.30 | 0.49 |
| Lys | 209 | A | A | . | . | . | . | . | −0.50 | 0.61 | . | . | . | −0.60 | 0.49 |
| Lys | 210 | A | A | . | . | . | . | . | −0.44 | 0.36 | * | . | . | −0.30 | 0.91 |
| Val | 211 | A | A | . | . | . | . | . | −0.44 | 0.43 | * | * | . | −0.45 | 1.36 |
| Leu | 212 | . | A | B | . | . | . | . | 0.41 | 0.20 | * | * | . | −0.30 | 0.56 |
| Pro | 213 | . | A | B | . | . | . | . | 0.36 | 0.20 | * | . | . | −0.30 | 0.56 |
| Tyr | 214 | . | . | . | B | T | . | . | −0.58 | 0.63 | * | . | . | −0.20 | 0.75 |
| Leu | 215 | . | . | . | B | T | . | . | −1.29 | 0.67 | * | * | . | −0.20 | 0.64 |
| Lys | 216 | . | . | . | B | T | . | . | −0.73 | 0.56 | * | . | . | −0.20 | 0.22 |
| Gly | 217 | . | . | B | B | . | . | . | −0.27 | 0.51 | * | . | . | −0.60 | 0.19 |
| Ile | 218 | . | . | B | B | . | . | . | −0.40 | 0.19 | * | . | . | −0.30 | 0.23 |
| Cys | 219 | . | . | B | . | . | T | . | −0.50 | −0.07 | * | . | . | 0.70 | 0.11 |
| Ser | 220 | . | . | . | . | T | T | . | −0.03 | 0.36 | . | * | F | 0.65 | 0.11 |
| Gly | 221 | . | . | . | . | T | T | . | −0.08 | 0.36 | . | . | F | 0.65 | 0.16 |
| Gly | 222 | . | . | . | . | T | T | . | 0.06 | −0.33 | . | . | F | 1.25 | 0.49 |
| Gly | 223 | . | . | . | . | . | . | C | 0.94 | −0.47 | . | . | F | 0.85 | 0.57 |
| Gly | 224 | . | . | . | . | . | . | C | 1.72 | −0.86 | * | . | F | 1.15 | 0.99 |
| Asp | 225 | . | . | . | . | . | T | C | 1.17 | −1.29 | . | * | F | 1.50 | 1.97 |
| Pro | 226 | . | . | . | . | . | T | C | 1.51 | −1.07 | * | . | F | 1.84 | 1.47 |
| Glu | 227 | . | . | B | . | . | T | . | 1.97 | −1.50 | * | . | F | 1.98 | 2.49 |
| Arg | 228 | . | . | B | . | . | T | . | 2.01 | −1.93 | * | . | F | 2.32 | 2.92 |
| Val | 229 | . | . | . | . | T | . | . | 2.06 | −1.54 | * | . | F | 2.86 | 2.53 |
| Asp | 230 | . | . | . | . | T | T | . | 2.06 | −1.59 | * | . | F | 3.40 | 1.96 |
| Arg | 231 | . | . | . | . | T | T | . | 2.38 | −1.19 | * | * | F | 3.06 | 1.73 |
| Ser | 232 | . | . | . | . | T | T | . | 2.17 | −1.19 | * | . | F | 2.72 | 4.57 |
| Ser | 233 | . | . | . | . | T | T | . | 1.71 | −1.40 | * | * | F | 2.72 | 4.23 |
| Gln | 234 | . | . | . | . | . | . | C | 1.98 | −0.97 | * | * | F | 2.32 | 2.14 |
| Arg | 235 | . | . | . | . | . | T | C | 1.98 | −0.47 | * | * | F | 2.22 | 1.61 |
| Pro | 236 | . | . | . | . | . | T | C | 1.87 | −0.86 | * | * | F | 2.86 | 2.08 |
| Gly | 237 | . | . | . | . | T | T | . | 2.17 | −1.24 | . | * | F | 3.40 | 2.01 |
| Ala | 238 | . | . | . | . | . | T | C | 1.61 | −1.24 | . | * | F | 2.86 | 1.65 |
| Glu | 239 | A | . | . | . | . | . | . | 0.80 | −0.60 | . | * | F | 1.97 | 0.79 |
| Asp | 240 | A | . | . | . | . | . | . | 0.69 | −0.34 | . | * | F | 1.33 | 0.66 |
| Asn | 241 | A | . | . | . | . | . | . | 0.90 | −0.37 | * | . | . | 0.99 | 1.05 |
| Val | 242 | A | . | . | . | . | . | . | 0.36 | −0.87 | * | . | . | 0.95 | 1.05 |
| Leu | 243 | A | . | . | . | . | . | . | 0.09 | −0.19 | * | . | . | 0.50 | 0.44 |
| Asn | 244 | A | . | . | B | . | . | . | −0.21 | 0.46 | * | . | . | −0.60 | 0.20 |
| Glu | 245 | A | . | . | B | . | . | . | −1.10 | 0.44 | * | . | . | −0.60 | 0.37 |
| Ile | 246 | A | . | . | B | . | . | . | −1.91 | 0.49 | * | . | . | −0.60 | 0.31 |
| Val | 247 | A | . | . | B | . | . | . | −1.06 | 0.49 | * | . | . | −0.60 | 0.16 |
| Ser | 248 | . | . | B | B | . | . | . | −0.46 | 0.49 | * | . | . | −0.60 | 0.16 |
| Ile | 249 | . | . | B | B | . | . | . | −0.77 | 0.91 | * | . | . | −0.60 | 0.35 |
| Leu | 250 | . | . | . | B | . | . | C | −0.77 | 0.71 | . | . | . | −0.40 | 0.69 |
| Gln | 251 | . | . | . | . | . | T | C | −0.73 | 0.47 | . | . | F | 0.15 | 0.89 |
| Pro | 252 | . | . | . | . | . | T | C | −0.09 | 0.73 | . | . | F | 0.15 | 0.94 |
| Thr | 253 | . | . | . | . | . | T | C | 0.21 | 0.47 | . | . | F | 0.30 | 1.76 |
| Gln | 254 | . | . | . | . | . | T | C | 1.10 | −0.21 | . | . | F | 1.20 | 1.76 |
| Val | 255 | . | A | . | . | . | . | C | 1.91 | −0.21 | . | . | F | 0.80 | 1.97 |
| Pro | 256 | . | A | . | . | . | . | C | 1.31 | −0.64 | . | . | F | 1.10 | 2.37 |
| Glu | 257 | A | A | . | . | . | . | . | 1.52 | −0.51 | . | * | F | 0.90 | 1.35 |
| Gln | 258 | A | A | . | . | . | . | . | 0.98 | −0.91 | . | * | F | 0.90 | 3.16 |
| Glu | 259 | A | A | . | . | . | . | . | 0.98 | −0.91 | . | * | F | 0.90 | 1.51 |
| Met | 260 | A | A | . | . | . | . | . | 1.83 | −0.94 | . | * | F | 0.90 | 1.51 |
| Glu | 261 | A | A | . | . | . | . | . | 1.83 | −0.94 | . | * | . | 0.75 | 1.51 |
| Val | 262 | A | A | . | . | . | . | . | 1.24 | −0.91 | . | * | F | 0.90 | 1.35 |
| Gln | 263 | A | A | . | . | . | . | . | 1.24 | −0.41 | . | * | F | 0.60 | 1.38 |
| Glu | 264 | A | A | . | . | . | . | . | 1.03 | −1.03 | . | * | F | 0.90 | 1.38 |
| Pro | 265 | A | A | . | . | . | . | . | 1.32 | −0.60 | . | * | F | 1.18 | 2.88 |
| Ala | 266 | A | A | . | . | . | . | . | 0.98 | −0.76 | . | * | F | 1.46 | 2.40 |
| Glu | 267 | A | . | . | . | . | T | . | 0.98 | −0.73 | . | * | F | 2.14 | 1.37 |
| Pro | 268 | A | . | . | . | . | T | . | 0.98 | −0.09 | . | . | F | 1.97 | 0.66 |
| Thr | 269 | . | . | . | . | T | T | . | 0.38 | −0.11 | . | . | F | 2.80 | 1.05 |
| Gly | 270 | A | . | . | . | . | T | . | −0.22 | 0.00 | . | . | F | 1.37 | 0.60 |
| Val | 271 | A | . | . | . | . | . | . | 0.07 | 0.69 | . | . | . | 0.44 | 0.32 |
| Asn | 272 | . | . | B | . | . | . | . | −0.14 | 0.64 | . | . | . | 0.16 | 0.30 |
| Met | 273 | . | . | B | . | . | . | . | −0.28 | 0.59 | . | . | . | 0.18 | 0.46 |
| Leu | 274 | . | . | . | . | . | . | C | 0.03 | 0.59 | . | . | . | 0.40 | 0.62 |
| Ser | 275 | . | . | . | . | . | T | C | 0.08 | −0.06 | . | . | F | 1.95 | 0.66 |
| Pro | 276 | . | . | . | . | . | T | C | 0.93 | −0.07 | . | . | F | 2.25 | 0.90 |
| Gly | 277 | . | . | . | . | . | T | C | 0.90 | −0.69 | . | . | F | 3.00 | 1.89 |
| Glu | 278 | A | . | . | . | . | T | . | 0.69 | −0.87 | . | . | F | 2.50 | 1.92 |
| Ser | 279 | A | A | . | . | . | . | . | 0.69 | −0.57 | . | . | F | 1.80 | 1.02 |
| Glu | 280 | A | A | . | . | . | . | . | 0.99 | −0.31 | . | . | F | 1.05 | 0.85 |
| His | 281 | A | A | . | . | . | . | . | 0.99 | −0.74 | . | . | F | 1.05 | 0.85 |
| Leu | 282 | A | A | . | . | . | . | . | 0.74 | −0.31 | . | . | . | 0.30 | 0.98 |
| Leu | 283 | A | A | . | . | . | . | . | 0.74 | −0.20 | . | . | . | 0.30 | 0.57 |
| Glu | 284 | A | A | . | . | . | . | . | 0.46 | −0.20 | . | . | F | 0.45 | 0.73 |
| Pro | 285 | A | A | . | . | . | . | . | 0.46 | −0.20 | . | . | F | 0.45 | 0.89 |
| Ala | 286 | A | A | . | . | . | . | . | 0.60 | −0.89 | . | . | F | 0.90 | 1.88 |
| Glu | 287 | A | A | . | . | . | . | . | 1.11 | −1.57 | . | . | F | 0.90 | 2.13 |
| Ala | 288 | A | A | . | . | . | . | . | 1.92 | −1.19 | . | . | F | 0.90 | 1.84 |
| Glu | 289 | A | A | . | . | . | . | . | 2.03 | −1.21 | * | . | F | 0.90 | 3.16 |
| Arg | 290 | A | A | . | . | . | . | . | 2.36 | −1.71 | * | . | F | 0.90 | 3.57 |
| Ser | 291 | A | . | . | . | . | T | . | 3.06 | −1.71 | * | . | F | 1.30 | 6.92 |
| Gln | 292 | A | . | . | . | . | T | . | 2.24 | −2.21 | * | . | F | 1.30 | 7.83 |
| Arg | 293 | A | . | . | . | . | T | . | 2.02 | −1.53 | . | . | F | 1.30 | 3.30 |
| Arg | 294 | A | . | . | . | . | T | . | 1.17 | −0.84 | . | . | F | 1.30 | 2.03 |
| Arg | 295 | . | . | . | B | T | . | . | 0.84 | −0.59 | . | * | F | 1.15 | 0.87 |
| Leu | 296 | . | . | B | B | . | . | . | 0.56 | −0.56 | . | * | . | 0.60 | 0.69 |
| Leu | 297 | . | . | B | B | . | . | . | 0.56 | −0.06 | . | * | . | 0.30 | 0.35 |
| Val | 298 | . | . | . | B | . | . | C | 0.44 | 0.34 | * | * | . | 0.20 | 0.29 |
| Pro | 299 | . | . | . | . | . | T | C | −0.01 | 0.34 | * | . | . | 0.90 | 0.61 |
| Ala | 300 | . | . | . | . | . | T | C | −0.12 | 0.09 | * | * | F | 1.35 | 0.73 |
| Asn | 301 | . | . | . | . | . | T | C | 0.48 | −0.60 | . | . | F | 2.70 | 1.65 |
| Glu | 302 | . | . | . | . | . | T | C | 0.98 | −0.81 | . | . | F | 3.00 | 1.65 |
| Gly | 303 | . | . | . | . | . | . | C | 1.83 | −0.76 | . | . | F | 2.50 | 2.35 |
| Asp | 304 | . | . | . | . | . | T | C | 1.73 | −1.26 | . | . | F | 2.40 | 2.54 |
| Pro | 305 | . | . | . | . | . | T | C | 1.51 | −1.17 | . | * | F | 2.10 | 2.11 |
| Thr | 306 | A | . | . | . | . | T | . | 1.62 | −0.49 | . | * | F | 1.30 | 1.76 |
| Glu | 307 | A | . | . | . | . | T | . | 1.62 | −0.91 | * | * | F | 1.30 | 2.07 |
| Thr | 308 | A | . | . | B | . | . | . | 1.30 | −0.51 | * | * | F | 0.90 | 2.31 |
| Leu | 309 | A | . | . | B | . | . | . | 0.60 | −0.37 | * | * | F | 0.45 | 0.86 |
| Arg | 310 | A | . | . | B | . | . | . | 0.81 | −0.07 | * | * | . | 0.30 | 0.43 |
| Gln | 311 | A | . | . | B | . | . | . | 1.12 | −0.07 | * | * | . | 0.30 | 0.50 |
| Cys | 312 | A | . | . | . | . | T | . | 0.42 | −0.56 | * | * | . | 1.15 | 1.01 |
| Phe | 313 | A | . | . | . | . | T | . | 0.14 | −0.46 | * | * | . | 0.70 | 0.45 |
| Asp | 314 | . | . | . | . | T | T | . | 0.96 | 0.04 | * | * | . | 0.50 | 0.26 |
| Asp | 315 | A | . | . | . | . | T | . | 0.03 | −0.36 | * | * | . | 0.70 | 0.81 |
| Phe | 316 | A | A | . | . | . | . | . | −0.82 | −0.24 | * | . | . | 0.30 | 0.77 |
| Ala | 317 | A | A | . | . | . | . | . | −0.37 | −0.39 | * | . | . | 0.30 | 0.34 |
| Asp | 318 | A | A | . | . | . | . | . | −0.37 | 0.04 | * | * | . | −0.30 | 0.32 |
| Leu | 319 | A | A | . | . | . | . | . | −0.37 | 0.83 | . | . | . | −0.60 | 0.32 |
| Val | 320 | . | A | . | . | . | . | C | −0.67 | 0.04 | . | . | . | −0.10 | 0.52 |
| Pro | 321 | . | A | . | . | . | . | C | −0.26 | −0.07 | . | . | . | 0.50 | 0.42 |
| Phe | 322 | . | . | . | . | T | T | . | 0.33 | 0.84 | . | . | . | 0.20 | 0.54 |
| Asp | 323 | A | . | . | . | . | T | . | 0.12 | 0.16 | . | . | . | 0.25 | 1.25 |
| Ser | 324 | A | . | . | . | . | T | . | 0.12 | −0.06 | . | . | F | 1.00 | 1.25 |
| Trp | 325 | A | . | . | . | . | T | . | 0.38 | 0.20 | * | * | F | 0.40 | 1.19 |
| Glu | 326 | A | A | . | . | . | . | . | 0.70 | 0.03 | * | . | F | −0.15 | 0.71 |
| Pro | 327 | A | A | . | . | . | . | . | 1.44 | 0.03 | * | . | . | −0.15 | 1.03 |
| Leu | 328 | A | A | . | . | . | . | . | 0.63 | −0.36 | * | . | . | 0.45 | 1.96 |
| Met | 329 | A | A | . | . | . | . | . | 0.59 | −0.59 | * | . | . | 0.60 | 0.93 |
| Arg | 330 | A | A | . | . | . | . | . | 0.07 | −0.16 | * | . | . | 0.30 | 0.60 |
| Lys | 331 | A | A | . | . | . | . | . | −0.53 | 0.10 | * | . | . | −0.30 | 0.60 |
| Leu | 332 | A | A | . | . | . | . | . | −0.32 | 0.03 | * | . | . | −0.30 | 0.60 |
| Gly | 333 | A | A | . | . | . | . | . | 0.49 | −0.59 | * | . | . | 0.60 | 0.51 |
| Leu | 334 | A | A | . | . | . | . | . | 1.09 | −0.19 | * | . | . | 0.30 | 0.41 |
| Met | 335 | A | A | . | . | . | . | . | 0.09 | −0.19 | * | * | . | 0.30 | 0.86 |
| Asp | 336 | A | A | . | . | . | . | . | 0.09 | −0.19 | . | * | F | 0.45 | 0.61 |
| Asn | 337 | A | A | . | . | . | . | . | 0.04 | −0.61 | * | * | F | 0.90 | 1.48 |
| Glu | 338 | A | A | . | . | . | . | . | −0.20 | −0.66 | * | * | F | 0.90 | 1.11 |
| Ile | 339 | A | A | . | . | . | . | . | 0.66 | −0.77 | * | * | F | 0.75 | 0.67 |
| Lys | 340 | A | A | . | . | . | . | . | 0.67 | −0.77 | . | * | F | 0.75 | 0.83 |
| Val | 341 | A | A | . | . | . | . | . | 0.67 | −0.67 | . | * | . | 0.60 | 0.49 |
| Ala | 342 | A | A | . | . | . | . | . | 0.08 | −0.67 | . | . | . | 0.75 | 1.20 |
| Lys | 343 | A | A | . | . | . | . | . | −0.51 | −0.86 | . | * | . | 0.60 | 0.61 |
| Ala | 344 | A | A | . | . | . | . | . | 0.03 | −0.36 | . | * | . | 0.30 | 0.83 |
| Glu | 345 | A | A | . | . | . | . | . | −0.04 | −0.57 | * | . | . | 0.60 | 0.81 |
| Ala | 346 | A | A | . | . | . | . | . | 0.92 | −0.57 | * | . | . | 0.60 | 0.55 |
| Ala | 347 | A | A | . | . | . | . | . | 1.51 | −0.57 | . | * | . | 0.75 | 1.07 |
| Gly | 348 | A | . | . | . | . | . | . | 1.16 | −1.07 | . | * | . | 0.95 | 1.03 |
| His | 349 | A | . | . | . | . | T | . | 0.93 | −0.59 | . | . | . | 1.15 | 1.47 |
| Arg | 350 | A | . | . | . | . | T | . | 0.69 | −0.40 | . | . | F | 1.00 | 1.20 |
| Asp | 351 | A | . | . | . | . | T | . | 0.97 | −0.14 | . | . | F | 1.00 | 1.90 |
| Thr | 352 | A | . | . | . | . | T | . | 0.96 | −0.09 | . | . | F | 1.00 | 2.02 |
| Leu | 353 | A | . | . | B | . | . | . | 0.49 | 0.03 | . | . | . | −0.15 | 1.02 |
| Tyr | 354 | A | . | . | B | . | . | . | −0.37 | 0.71 | . | * | . | −0.60 | 0.50 |
| Thr | 355 | A | . | . | B | . | . | . | −0.43 | 1.40 | . | * | . | −0.60 | 0.24 |
| Met | 356 | A | . | . | B | . | . | . | −0.72 | 0.91 | * | . | . | −0.60 | 0.59 |
| Leu | 357 | A | . | . | B | . | . | . | −1.27 | 1.14 | * | . | . | −0.60 | 0.40 |
| Ile | 358 | A | . | . | B | . | . | . | −0.46 | 1.03 | * | * | . | −0.60 | 0.20 |
| Lys | 359 | A | . | . | B | . | . | . | −0.17 | 0.94 | * | * | . | −0.60 | 0.33 |
| Trp | 360 | A | . | . | B | . | . | . | −0.17 | 0.33 | * | * | . | 0.00 | 0.81 |
| Val | 361 | A | . | . | B | . | . | . | 0.09 | 0.13 | * | * | . | 0.45 | 1.66 |
| Asn | 362 | . | . | . | . | . | T | C | 1.01 | −0.13 | * | . | F | 1.95 | 0.82 |
| Lys | 363 | . | . | . | . | . | T | C | 1.90 | −0.13 | * | * | F | 2.40 | 1.53 |
| Thr | 364 | . | . | . | . | . | T | C | 1.27 | −1.04 | * | . | F | 3.00 | 3.44 |
| Gly | 365 | . | . | . | . | . | T | C | 1.26 | −1.19 | * | . | F | 2.70 | 2.16 |
| Arg | 366 | . | A | . | . | T | . | . | 1.26 | −1.20 | * | . | F | 2.20 | 1.45 |
| Asp | 367 | . | A | . | . | . | . | C | 1.22 | −0.56 | * | . | F | 1.55 | 0.75 |
| Ala | 368 | A | A | . | . | . | . | . | 0.87 | −0.54 | . | . | F | 1.20 | 1.03 |
| Ser | 369 | A | A | . | . | . | . | . | 0.37 | −0.49 | . | . | . | 0.30 | 0.76 |
| Val | 370 | A | A | . | . | . | . | . | −0.10 | 0.20 | . | * | . | −0.30 | 0.37 |
| His | 371 | A | A | . | . | . | . | . | −0.21 | 0.89 | . | * | . | −0.60 | 0.30 |
| Thr | 372 | A | A | . | . | . | . | . | −0.80 | 0.39 | * | * | . | −0.30 | 0.38 |
| Leu | 373 | A | A | . | . | . | . | . | −1.02 | 0.50 | * | * | . | −0.60 | 0.52 |
| Leu | 374 | A | A | . | . | . | . | . | −0.72 | 0.54 | * | . | . | −0.60 | 0.31 |
| Asp | 375 | A | A | . | . | . | . | . | −0.18 | 0.04 | * | . | . | −0.30 | 0.38 |
| Ala | 376 | A | A | . | . | . | . | . | −0.96 | 0.04 | * | . | . | −0.30 | 0.66 |
| Leu | 377 | A | A | . | . | . | . | . | −0.99 | 0.04 | * | . | . | −0.30 | 0.66 |
| Glu | 378 | A | A | . | . | . | . | . | −0.18 | −0.21 | * | . | . | 0.30 | 0.39 |
| Thr | 379 | A | A | . | . | . | . | . | 0.74 | −0.21 | * | * | F | 0.45 | 0.67 |
| Leu | 380 | A | A | . | . | . | . | . | −0.07 | −0.71 | * | . | F | 0.90 | 1.59 |
| Gly | 381 | A | A | . | . | . | . | . | −0.07 | −0.71 | * | . | F | 0.75 | 0.76 |
| Glu | 382 | A | A | . | . | . | . | . | 0.79 | −0.21 | * | . | F | 0.45 | 0.53 |
| Arg | 383 | A | A | . | . | . | . | . | 0.79 | −0.70 | * | . | F | 0.90 | 1.28 |
| Leu | 384 | A | A | . | . | . | . | . | 1.14 | −0.99 | * | * | F | 0.90 | 2.24 |
| Ala | 385 | A | A | . | . | . | . | . | 1.07 | −1.41 | * | * | F | 0.90 | 2.59 |
| Lys | 386 | A | A | . | . | . | . | . | 1.41 | −0.73 | * | . | F | 0.75 | 0.93 |
| Gln | 387 | A | A | . | . | . | . | . | 1.41 | −0.73 | * | * | F | 0.90 | 1.95 |
| Lys | 388 | A | A | . | . | . | . | . | 1.27 | −1.41 | * | * | F | 0.90 | 3.22 |
| Ile | 389 | A | A | . | . | . | . | . | 1.27 | −1.41 | . | * | F | 0.90 | 2.19 |
| Glu | 390 | A | A | . | . | . | . | . | 1.04 | −0.73 | * | * | F | 0.90 | 1.04 |
| Asp | 391 | A | A | . | . | . | . | . | 0.70 | −0.44 | . | * | F | 0.45 | 0.43 |
| His | 392 | A | A | . | . | . | . | . | 0.40 | −0.06 | * | * | . | 0.30 | 0.82 |
| Leu | 393 | A | A | . | . | . | . | . | 0.01 | −0.36 | * | * | . | 0.30 | 0.64 |
| Leu | 394 | A | A | . | . | . | . | . | 0.94 | 0.07 | * | * | F | −0.15 | 0.38 |
| Ser | 395 | A | . | . | . | . | T | . | 0.24 | 0.07 | * | * | F | 0.25 | 0.55 |
| Ser | 396 | A | . | . | . | . | T | . | −0.36 | 0.36 | * | * | F | 0.25 | 0.58 |
| Gly | 397 | . | . | . | . | T | T | . | −0.57 | 0.29 | . | . | F | 0.65 | 0.70 |
| Lys | 398 | A | . | . | . | . | T | . | −0.57 | 0.36 | . | . | F | 0.25 | 0.82 |
| Phe | 399 | A | A | . | . | . | . | . | 0.24 | 0.66 | . | . | . | −0.60 | 0.50 |
| Met | 400 | . | A | B | . | . | . | . | 0.20 | 0.27 | . | * | . | −0.30 | 0.88 |
| Tyr | 401 | . | A | B | . | . | . | . | 0.50 | 0.27 | . | * | . | −0.30 | 0.44 |
| Leu | 402 | A | A | . | . | . | . | . | 0.26 | 0.67 | . | * | . | −0.60 | 0.81 |
| Glu | 403 | A | A | . | . | . | . | . | 0.21 | 0.39 | . | * | . | −0.30 | 0.82 |
| Gly | 404 | A | . | . | . | . | . | . | 0.61 | −0.23 | . | * | F | 0.65 | 0.88 |
| Asn | 405 | A | . | . | . | . | T | . | 0.62 | −0.60 | . | * | F | 1.30 | 1.43 |
| Ala | 406 | A | . | . | . | . | T | . | 0.27 | −0.79 | . | * | F | 1.15 | 0.83 |
| Asp | 407 | A | . | . | . | . | T | . | 0.78 | −0.17 | . | * | F | 0.85 | 0.83 |
| Ser | 408 | A | . | . | . | . | T | . | 0.39 | −0.21 | . | * | F | 0.85 | 0.69 |
| Ala | 409 | A | . | . | . | . | . | . | 0.34 | −0.19 | . | * | . | 0.50 | 0.88 |
| Met | 410 | A | . | . | . | . | . | . | −0.04 | −0.26 | . | . | . | 0.50 | 0.67 |
| Ser | 411 | A | . | . | . | . | . | . | 0.16 | 0.17 | . | . | . | −0.10 | 0.64 |

In another aspect, the invention provides an antibody that binds a peptide or polypeptide comprising, or alternatively, consisting of, one, two, three, four, five or more, epitope-bearing portions of a TR7. The epitope of this polypeptide portion is an immunogenic or antigenic epitope of a polypeptide described herein. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, J. G. Sutcliffe et al., “Antibodies That React With Predetermined Sites on Proteins,” Science 219:660-666 (1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides are therefore useful to raise antibodies, including monoclonal antibodies, that bind to a TR7 polypeptide. See, for instance, Wilson et al., Cell 37:767-778 (1984) at 777. Antigenic epitope-bearing peptides and polypeptides preferably contain a sequence of at least seven, more preferably at least nine and most preferably between at least about 15 to about 30 amino acids contained within the amino acid sequence of SEQ ID NO:3.

Antibodies of the invention may bind one or more antigenic TR7 polypeptides or peptides including, but not limited to: a polypeptide comprising, or alternatively consisting of, amino acid residues from about 62 to about 110 of SEQ ID NO:3, about 119 to about 164 of SEQ ID NO:3, about 224 to about 271 of SEQ ID NO:3, about 275 to about 370 of SEQ ID NO:3, about 69 to about 80 of SEQ ID NO:3, about 88 to about 95 of SEQ ID NO:3, about 99 to about 103 of SEQ ID NO:3, about 119 to about 123 of SEQ ID NO:3, about 130 to about 135 of SEQ ID NO:3, about 152 to about 163 of SEQ ID NO:3, about 226 to about 238 of SEQ ID NO:3, about 275 to about 279 of SEQ ID NO:3, about 301 to about 305 of SEQ ID NO:3, and/or about 362 to about 367 of SEQ ID NO:3. In this context “about” includes the particularly recited ranges, larger or smaller by several (5, 4, 3, 2, or 1) nucleotides, at either terminus or at both termini. As indicated above, the inventors have determined that the above polypeptide fragments are antigenic regions of the TR7 receptor protein.

Epitope-bearing TR7 peptides and polypeptides may be produced by any conventional means. R. A. Houghten, “General Method for the Rapid Solid-Phase Synthesis of Large Numbers of Peptides: Specificity of Antigen-Antibody Interaction at the Level of Individual Amino Acids,” Proc. Natl. Acad. Sci. USA 82:5131-5135 (1985). This “Simultaneous Multiple Peptide Synthesis (SMPS)” process is further described in U.S. Pat. No. 4,631,211 to Houghten et al. (1986).

As one of skill in the art will appreciate, TR7 receptor polypeptides and the epitope-bearing fragments thereof described herein (e.g., corresponding to a portion of the extracellular domain, such as, for example, amino acid residues 52 to 184 of SEQ ID NO:3 can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric TR7 protein or protein fragment alone (Fountoulakis et al., J. Biochem. 270:3958-3964 (1995)). TR7 fusion proteins may be used as an immunogen to elicit anti-TR7 antibodies. Thus, antibodies of the invention may bind fusion proteins that comprise all or a portion of a TRAIL receptor polypeptide such as TR7.

Recombinant DNA technology known to those skilled in the art can be used to create novel mutant proteins or “muteins” including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Antibodies of the present invention may also bind such modified TR7 polypeptides or TR7 polypeptide fragments or variants.

For instance, for many proteins, including the extracellular domain of a membrane associated protein or the mature form(s) of a secreted protein, it is known in the art that one or more amino acids may be deleted from the N-terminus or C-terminus without substantial loss of biological function or loss of the ability to be bound by a specific antibody. However, even if deletion of one or more amino acids from the N-terminus or C-terminus of a protein results in modification or loss of one or more biological functions of the protein, other TR7 functional activities may still be retained. For example, in many instances, the ability of the shortened protein to induce and/or bind to antibodies which recognize TR7 (preferably antibodies that bind specifically to TR7) will retained irrespective of the size or location of the deletion. In fact, polypeptides composed of as few as six TR7 amino acid residues may often evoke an immune response. Whether a particular polypeptide lacking N-terminal and/or C-terminal residues of a complete protein retains such immunologic activities can readily be determined by routine methods described herein and otherwise known in the art.

As mentioned above, even if deletion of one or more amino acids from the N-terminus of a protein results in modification or loss of one or more biological functions of the protein, other functional activities (e.g., biological activities, ability to multimerize, ability to bind TR7 ligand) may still be retained. For example, the ability of shortened TR7 polypeptides to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a TR7 polypeptide with a large number of deleted N-terminal amino acid residues may retain some biological or immunogenic activities.

Accordingly, the present invention further provides antibodies that bind polypeptides having one or more residues deleted from the amino terminus of the TR7 amino acid sequence shown in SEQ ID NO:3 up to the alanine residue at position number 406 and polynucleotides encoding such polypeptides. In particular, the present invention provides antibodies that bind polypeptides comprising the amino acid sequence of residues n⁵-411 of SEQ ID NO:3 where n⁵ is an integer from 2 to 406 corresponding to the position of the amino acid residue in SEQ ID NO:3.

More in particular, the invention provides antibodies that bind polypeptides comprising, or alternatively consisting of, the amino acid sequence of residues: E-2 to S-411; Q-3 to S-411; R-4 to S-411; G-5 to S-411; Q-6 to S-411; N-7 to S-411; A-8 to S-411; P-9 to S-411; A-10 to S-411; A-11 to S-411; S-12 to S-411; G-13 to S-411; A-14 to S-411; R-15 to S-411; K-16 to S-411; R-17 to S-411; H-18 to S-411; G-19 to S-411; P-20 to S-411; G-21 to S-411; P-22 to S-411; R-23 to S-411; E-24 to S-411; A-25 to S-411; R-26 to S-411; G-27 to S-411; A-28 to S-411; R-29 to S-411; P-30 to S-411; G-31 to S-411; P-32 to S-411; R-33 to S-411; V-34 to S-411; P-35 to S-411; K-36 to S-411; T-37 to S-411; L-38 to S-411; V-39 to S-411; L-40 to S-411; V-41 to S-411; V-42 to S-411; A-43 to S-411; A-44 to S-411; V-45 to S-411; L-46 to S-411; L-47 to S-411; L-48 to S-411; V-49 to S-411; S-50 to S-411; A-51 to S-411; E-52 to S-411; S-53 to S-411; A-54 to S-411; L-55 to S-411; I-56 to S-411; T-57 to S-411; Q-58 to S-411; Q-59 to S-411; D-60 to S-411; L-61 to S-411; A-62 to S-411; P-63 to S-411; Q-64 to S-411; Q-65 to S-411; R-66 to S-411; A-67 to S-411; A-68 to S-411; P-69 to S-411; Q-70 to S-411; Q-71 to S-411; K-72 to S-411; R-73 to S-411; S-74 to S-411; S-75 to S-411; P-76 to S-411; S-77 to S-411; E-78 to S-411; G-79 to S-411; L-80 to S-411; C-81 to S-411; P-82 to S-411; P-83 to S-411; G-84 to S-411; H-85 to S-411; H-86 to S-411; I-87 to S-411; S-88 to S-411; E-89 to S-411; D-90 to S-411; G-91 to S-411; R-92 to S-411; D-93 to S-411; C-94 to S-411; I-95 to S-411; S-96 to S-411; C-97 to S-411; K-98 to S-411; Y-99 to S-411; G-100 to S-411; Q-101 to S-411; D-102 to S-411; Y-103 to S-411; S-104 to S-411; T-105 to S-411; H-106 to S-411; W-107 to S-411; N-108 to S-411; D-109 to S-411; L-100 to S-411; L-111 to S-411; F-112 to S-411; C-113 to S-411; L-114 to S-411; R-115 to S-411; C-116 to S-411; T-117 to S-411; R-118 to S-411; C-119 to S-411; D-120 to S-411; S-121 to S-411; G-122 to S-411; E-123 to S-411; V-124 to S-411; E-125 to S-411; L-126 to S-411; S-127 to S-411; P-128 to S-411; C-129 to S-411; T-130 to S-411; T-131 to S-411; T-132 to S-411; R-133 to S-411; N-134 to S-411; T-135 to S-411; V-136 to S-411; C-137 to S-411; Q-138 to S-411; C-139 to S-411; E-140 to S-411; E-141 to S-411; G-142 to S-411; T-143 to S-411; F-144 to S-411; R-145 to S-411; E-146 to S-411; E-147 to S-411; D-148 to S-411; S-149 to S-411; P-150 to S-411; E-151 to S-411; M-152 to S-411; C-153 to S-411; R-154 to S-411; K-155 to S-411; C-156 to S-411; R-157 to S-411; T-158 to S-411; G-159 to S-411; C-160 to S-411; P-161 to S-411; R-162 to S-411; G-163 to S-411; M-164 to S-411; V-165 to S-411; K-166 to S-411; V-167 to S-411; G-168 to S-411; D-169 to S-411; C-170 to S-411; T-171 to S-411; P-172 to S-411; W-173 to S-411; S-174 to S-411; D-175 to S-411; I-176 to S-411; E-177 to S-411; C-178 to S-411; V-179 to S-411; H-180 to S-411; K-181 to S-411; E-182 to S-411; S-183 to S-411; G-184 to S-411; I-185 to S-411; I-186 to S-411; I-187 to S-411; G-188 to S-411; V-189 to S-411; T-190 to S-411; V-191 to S-411; A-192 to S-411; A-193 to S-411; V-194 to S-411; V-195 to S-411; L-196 to S-411; I-197 to S-411; V-198 to S-411; A-199 to S-411; V-200 to S-411; F-201 to S-411; V-202 to S-411; C-203 to S-411; K-204 to S-411; S-205 to S-411; L-206 to S-411; L-207 to S-411; W-208 to S-411; K-209 to S-411; K-210 to S-411; V-211 to S-411; L-212 to S-411; P-213 to S-411; Y-214 to S-411; L-215 to S-411; K-216 to S-411; G-217 to S-411; I-218 to S-411; C-219 to S-411; S-220 to S-411; G-221 to S-411; G-222 to S-411; G-223 to S-411; G-224 to S-411; D-225 to S-411; P-226 to S-411; E-227 to S-411; R-228 to S-411; V-229 to S-411; D-230 to S-411; R-231 to S-411; S-232 to S-411; S-233 to S-411; Q-234 to S-411; R-235 to S-411; P-236 to S-411; G-237 to S-411; A-238 to S-411; E-239 to S-411; D-240 to S-411; N-241 to S-411; V-242 to S-411; L-243 to S-411; N-244 to S-411; E-245 to S-411; I-246 to S-411; V-247 to S-411; S-248 to S-411; I-249 to S-411; L-250 to S-411; Q-251 to S-411; P-252 to S-411; T-253 to S-411; Q-254 to S-411; V-255 to S-411; P-256 to S-411; E-257 to S-411; Q-258 to S-411; E-259 to S-411; M-260 to S-411; E-261 to S-411; V-262 to S-411; Q-263 to S-411; E-264 to S-411; P-265 to S-411; A-266 to S-411; E-267 to S-411; P-268 to S-411; T-269 to S-411; G-270 to S-411; V-271 to S-411; N-272 to S-411; M-273 to S-411; L-274 to S-411; S-275 to S-411; P-276 to S-411; G-277 to S-411; E-278 to S-411; S-279 to S-411; E-280 to S-411; H-281 to S-411; L-282 to S-411; L-283 to S-411; E-284 to S-411; P-285 to S-411; A-286 to S-411; E-287 to S-411; A-288 to S-411; E-289 to S-411; R-290 to S-411; S-291 to S-411; Q-292 to S-411; R-293 to S-411; R-294 to S-411; R-295 to S-411; L-296 to S-411; L-297 to S-411; V-298 to S-411; P-299 to S-411; A-300 to S-411; N-301 to S-411; E-302 to S-411; G-303 to S-411; D-304 to S-411; P-305 to S-411; T-306 to S-411; E-307 to S-411; T-308 to S-411; L-309 to S-411; R-310 to S-411; Q-311 to S-411; C-312 to S-411; F-313 to S-411; D-314 to S-411; D-315 to S-411; F-316 to S-411; A-317 to S-411; D-318 to S-411; L-319 to S-411; V-320 to S-411; P-321 to S-411; F-322 to S-411; D-323 to S-411; S-324 to S-411; W-325 to S-411; E-326 to S-411; P-327 to S-411; L-328 to S-411; M-329 to S-411; R-330 to S-411; K-331 to S-411; L-332 to S-411; G-333 to S-411; L-334 to S-411; M-335 to S-411; D-336 to S-411; N-337 to S-411; E-338 to S-411; I-339 to S-411; K-340 to S-411; V-341 to S-411; A-342 to S-411; K-343 to S-411; A-344 to S-411; E-345 to S-411; A-346 to S-411; A-347 to S-411; G-348 to S-411; H-349 to S-411; R-350 to S-411; D-351 to S-411; T-352 to S-411; L-353 to S-41; Y-354 to S-411; T-355 to S-411; M-356 to S-411; L-357 to S-411; I-358 to S-411; K-359 to S-411; W-360 to S-411; V-361 to S-411; N-362 to S-411; K-363 to S-411; T-364 to S-411; G-365 to S-411; R-366 to S-411; D-367 to S-411; A-368 to S-411; S-369 to S-411; V-370 to S-411; H-371 to S-411; T-372 to S-411; L-373 to S-411; L-374 to S-411; D-375 to S-411; A-376 to S-411; L-377 to S-411; E-378 to S-411; T-379 to S-411; L-380 to S-411; G-381 to S-411; E-382 to S-411; R-383 to S-411; L-384 to S-411; A-385 to S-411; K-386 to S-411; Q-387 to S-411; K-388 to S-411; I-389 to S-411; E-390 to S-411; D-391 to S-411; H-392 to S-411; L-393 to S-411; L-394 to S-411; S-395 to S-411; S-396 to S-411; G-397 to S-411; K-398 to S-411; F-399 to S-411; M-400 to S-411; Y-401 to S-411; L-402 to S-411; E-403 to S-411; G-404 to S-411; N-405 to S-411; and/or A-406 to S-411 of the TR7 sequence shown in SEQ ID NO:3.

In another embodiment, N-terminal deletions of the TR7 polypeptide can be described by the general formula n⁶ to 184 where n⁶ is a number from 1 to 179 corresponding to the amino acid sequence identified in SEQ ID NO:3. In specific embodiments, antibodies of the invention bind N terminal deletions of the TR7 comprising, or alternatively consisting of, the amino acid sequence of residues: E-2 to G-184; Q-3 to G-184; R-4 to G-184; G-5 to G-184; Q-6 to G-184; N-7 to G-184; A-8 to G-184; P-9 to G-184; A-10 to G-184; A-11 to G-184; S-12 to G-184; G-13 to G-184; A-14 to G-184; R-15 to G-184; K-16 to G-184; R-17 to G-184; H-18 to G-184; G-19 to G-184; P-20 to G-184; G-21 to G-184; P-22 to G-184; R-23 to G-184; E-24 to G-184; A-25 to G-184; R-26 to G-184; G-27 to G-184; A-28 to G-184; R-29 to G-184; P-30 to G-184; G-31 to G-184; P-32 to G-184; R-33 to G-184; V-34 to G-184; P-35 to G-184; K-36 to G-184; T-37 to G-184; L-38 to G-184; V-39 to G-184; L-40 to G-184; V-41 to G-184; V-42 to G-184; A-43 to G-184; A-44 to G-184; V-45 to G-184; L-46 to G-184; L-47 to G-184; L-48 to G-184; V-49 to G-184; S-50 to G-184; A-51 to G-184; E-52 to G-184; S-53 to G-184; A-54 to G-184; L-55 to G-184; I-56 to G-184; T-57 to G-184; Q-58 to G-184; Q-59 to G-184; D-60 to G-184; L-61 to G-184; A-62 to G-184; P-63 to G-184; Q-64 to G-184; Q-65 to G-184; R-66 to G-184; A-67 to G-184; A-68 to G-184; P-69 to G-184; Q-70 to G-184; Q-71 to G-184; K-72 to G-184; R-73 to G-184; S-74 to G-184; S-75 to G-184; P-76 to G-184; S-77 to G-184; E-78 to G-184; G-79 to G-184; L-80 to G-184; C-81 to G-184; P-82 to G-184; P-83 to G-184; G-84 to G-184; H-85 to G-184; H-86 to G-184; I-87 to G-184; S-88 to G-184; E-89 to G-184; D-90 to G-184; G-91 to G-184; R-92 to G-184; D-93 to G-184; C-94 to G-184; I-95 to G-184; S-96 to G-184; C-97 to G-184; K-98 to G-184; Y-99 to G-184; G-100 to G-184; Q-101 to G-184; D-102 to G-184; Y-103 to G-184; S-104 to G-184; T-105 to G-184; H-106 to G-184; W-107 to G-184; N-108 to G-184; D-109 to G-184; L-110 to G-184; L-111 to G-184; F-112 to G-184; C-113 to G-184; L-114 to G-184; R-115 to G-184; C-116 to G-184; T-117 to G-184; R-118 to G-184; C-119 to G-184; D-120 to G-184; S-121 to G-184; G-122 to G-184; E-123 to G-184; V-124 to G-184; E-125 to G-184; L-126 to G-184; S-127 to G-184; P-128 to G-184; C-129 to G-184; T-130 to G-184; T-131 to G-184; T-132 to G-184; R-133 to G-184; N-134 to G-184; T-135 to G-184; V-136 to G-184; C-137 to G-184; Q-138 to G-184; C-139 to G-184; E-140 to G-184; E-141 to G-184; G-142 to G-184; T-143 to G-184; F-144 to G-184; R-145 to G-184; E-146 to G-184; E-147 to G-184; D-148 to G-184; S-149 to G-184; P-150 to G-184; E-151 to G-184; M-152 to G-184; C-153 to G-184; R-154 to G-184; K-155 to G-184; C-156 to G-184; R-157 to G-184; T-158 to G-184; G-159 to G-184; C-160 to G-184; P-161 to G-184; R-162 to G-184; G-163 to G-184; M-164 to G-184; V-165 to G-184; K-166 to G-184; V-167 to G-184; G-168 to G-184; D-169 to G-184; C-170 to G-184; T-171 to G-184; P-172 to G-184; W-173 to G-184; S-174 to G-184; D-175 to G-184; I-176 to G-184; E-177 to G-184; C-178 to G-184; and/or V-179 to G-184; of the TR7 extracellular domain sequence shown in SEQ ID NO:3.

Also as mentioned above, even if deletion of one or more amino acids from the C-terminus of a protein results in modification of loss of one or more biological functions of the protein, other functional activities (e.g., biological activities, ability to multimerize, ability to bind TR7 ligand (e.g., TRAIL)) may still be retained. For example, the ability of the shortened TR7 polypeptide to induce and/or bind to antibodies which recognize the complete or mature forms of the polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that a TR7 polypeptide with a large number of deleted C-terminal amino acid residues may retain some biological or immunogenic activities. In fact, peptides composed of as few as six TR7 amino acid residues may often evoke an immune response.

Accordingly, the present invention further provides antibodies that bind polypeptides having one or more residues deleted from the carboxy terminus of the amino acid sequence of the TR7 polypeptide shown in SEQ ID NO:3 up to the glutamic acid residue at position number 52. In particular, the present invention provides antibodies that bind polypeptides comprising the amino acid sequence of residues 52-m⁵ of SEQ ID NO:3, where m⁵ is an integer from 57 to 410 corresponding to the position of the amino acid residue in SEQ ID NO:3.

More in particular, the invention provides antibodies that bind polypeptides comprising, or alternatively consisting of, the amino acid sequence of residues: E-52 to M-410; E-52 to A-409; E-52 to S-408; E-52 to D-407; E-52 to A-406; E-52 to N-405; E-52 to G-404; E-52 to E-403; E-52 to L-402; E-52 to Y-401; E-52 to M-400; E-52 to F-399; E-52 to K-398; E-52 to G-397; E-52 to S-396; E-52 to S-395; E-52 to L-394; E-52 to L-393; E-52 to H-392; E-52 to D-391; E-52 to E-390; E-52 to I-389; E-52 to K-388; E-52 to Q-387; E-52 to K-386; E-52 to A-385; E-52 to L-384; E-52 to R-383; E-52 to E-382; E-52 to G-381; E-52 to L-380; E-52 to T-379; E-52 to E-378; E-52 to L-377; E-52 to A-376; E-52 to D-375; E-52 to L-374; E-52 to L-373; E-52 to T-372; E-52 to H-371; E-52 to V-370; E-52 to S-369; E-52 to A-368; E-52 to D-367; E-52 to R-366; E-52 to G-365; E-52 to T-364; E-52 to K-363; E-52 to N-362; E-52 to V-361; E-52 to W-360; E-52 to K-359; E-52 to I-358; E-52 to L-357; E-52 to M-356; E-52 to T-355; E-52 to Y-354; E-52 to L-353; E-52 to T-352; E-52 to D-351; E-52 to R-350; E-52 to H-349; E-52 to G-348; E-52 to A-347; E-52 to A-346; E-52 to E-345; E-52 to A-344; E-52 to K-343; E-52 to A-342; E-52 to V-341; E-52 to K-340; E-52 to I-339; E-52 to E-338; E-52 to N-337; E-52 to D-336; E-52 to M-335; E-52 to L-334; E-52 to G-333; E-52 to L-332; E-52 to K-331; E-52 to R-330; E-52 to M-329; E-52 to L-328; E-52 to P-327; E-52 to E-326; E-52 to W-325; E-52 to S-324; E-52 to D-323; E-52 to F-322; E-52 to P-321; E-52 to V-320; E-52 to L-319; E-52 to D-318; E-52 to A-317; E-52 to F-316; E-52 to D-315; E-52 to D-314; E-52 to F-313; E-52 to C-312; E-52 to Q-311; E-52 to R-310; E-52 to L-309; E-52 to T-308; E-52 to E-307; E-52 to T-306; E-52 to P-305; E-52 to D-304; E-52 to G-303; E-52 to E-302; E-52 to N-301; E-52 to A-300; E-52 to P-299; E-52 to V-298; E-52 to L-297; E-52 to L-296; E-52 to R-295; E-52 to R-294; E-52 to R-293; E-52 to Q-292; E-52 to S-291; E-52 to R-290; E-52 to E-289; E-52 to A-288; E-52 to E-287; E-52 to A-286; E-52 to P-285; E-52 to E-284; E-52 to L-283; E-52 to L-282; E-52 to H-281; E-52 to E-280; E-52 to S-279; E-52 to E-278; E-52 to G-277; E-52 to P-276; E-52 to S-275; E-52 to L-274; E-52 to M-273; E-52 to N-272; E-52 to V-271; E-52 to G-270; E-52 to T-269; E-52 to P-268; E-52 to E-267; E-52 to A-266; E-52 to P-265; E-52 to E-264; E-52 to Q-263; E-52 to V-262; E-52 to E-261; E-52 to M-260; E-52 to E-259; E-52 to Q-258; E-52 to E-257; E-52 to P-256; E-52 to V-255; E-52 to Q-254; E-52 to T-253; E-52 to P-252; E-52 to Q-251; E-52 to L-250; E-52 to I-249; E-52 to S-248; E-52 to V-247; E-52 to I-246; E-52 to E-245; E-52 to N-244; E-52 to L-243; E-52 to V-242; E-52 to N-241; E-52 to D-240; E-52 to E-239; E-52 to A-238; E-52 to G-237; E-52 to P-236; E-52 to R-235; E-52 to Q-234; E-52 to S-233; E-52 to S-232; E-52 to R-231; E-52 to D-230; E-52 to V-229; E-52 to R-228; E-52 to E-227; E-52 to P-226; E-52 to D-225; E-52 to G-224; E-52 to G-223; E-52 to G-222; E-52 to G-221; E-52 to S-220; E-52 to C-219; E-52 to I-218; E-52 to G-217; E-52 to K-216; E-52 to L-215; E-52 to Y-214; E-52 to P-213; E-52 to L-212; E-52 to V-211; E-52 to K-210; E-52 to K-209; E-52 to W-208; E-52 to L-207; E-52 to L-206; E-52 to S-205; E-52 to K-204; E-52 to C-203; E-52 to V-202; E-52 to F-201; E-52 to V-200; E-52 to A-199; E-52 to V-198; E-52 to I-197; E-52 to L-196; E-52 to V-195; E-52 to V-194; E-52 to A-193; E-52 to A-192; E-52 to V-191; E-52 to T-190; E-52 to V-189; E-52 to G-188; E-52 to I-187; E-52 to I-186; E-52 to I-185; E-52 to G-184; E-52 to S-183; E-52 to E-182; E-52 to K-181; E-52 to H-180; E-52 to V-179; E-52 to C-178; E-52 to E-177; E-52 to I-176; E-52 to D-175; E-52 to S-174; E-52 to W-173; E-52 to P-172; E-52 to T-171; E-52 to C-170; E-52 to D-169; E-52 to G-168; E-52 to V-167; E-52 to K-166; E-52 to V-165; E-52 to M-164; E-52 to G-163; E-52 to R-162; E-52 to P-161; E-52 to C-160; E-52 to G-159; E-52 to T-158; E-52 to R-157; E-52 to C-156; E-52 to K-155; E-52 to R-154; E-52 to C-153; E-52 to M-152; E-52 to E-151; E-52 to P-150; E-52 to S-149; E-52 to D-148; E-52 to E-147; E-52 to E-146; E-52 to R-145; E-52 to F-144; E-52 to T-143; E-52 to G-142; E-52 to E-141; E-52 to E-140; E-52 to C-139; E-52 to Q-138; E-52 to C-137; E-52 to V-136; E-52 to T-135; E-52 to N-134; E-52 to R-133; E-52 to T-132; E-52 to T-131; E-52 to T-130; E-52 to C-129; E-52 to P-128; E-52 to S-127; E-52 to L-126; E-52 to E-125; E-52 to V-124; E-52 to E-123; E-52 to G-122; E-52 to S-121; E-52 to D-120; E-52 to C-119; E-52 to R-118; E-52 to T-117; E-52 to C-116; E-52 to R-115; E-52 to L-114; E-52 to C-113; E-52 to F-12; E-52 to L-11; E-52 to L-110; E-52 to D-109; E-52 to N-108; E-52 to W-107; E-52 to H-106; E-52 to T-105; E-52 to S-104; E-52 to Y-103; E-52 to D-102; E-52 to Q-101; E-52 to G-100; E-52 to Y-99; E-52 to K-98; E-52 to C-97; E-52 to S-96; E-52 to I-95; E-52 to C-94; E-52 to D-93; E-52 to R-92; E-52 to G-91; E-52 to D-90; E-52 to E-89; E-52 to S-88; E-52 to I-87; E-52 to H-86; E-52 to H-85; E-52 to G-84; E-52 to P-83; E-52 to P-82; E-52 to C-81; E-52 to L-80; E-52 to G-79; E-52 to E-78; E-52 to S-77; E-52 to P-76; E-52 to S-75; E-52 to S-74; E-52 to R-73; E-52 to K-72; E-52 to Q-71; E-52 to Q-70; E-52 to P-69; E-52 to A-68; E-52 to A-67; E-52 to R-66; E-52 to Q-65; E-52 to Q-64; E-52 to P-63; E-52 to A-62; E-52 to L-61; E-52 to D-60; E-52 to Q-59; E-52 to Q-58; and/or E-52 to T-57; of the TR7 sequence shown in SEQ ID NO:3.

In another embodiment, antibodies of the invention bind C-terminal deletions of the TR7 polypeptide that can be described by the general formula 52-m⁶ where m⁶ is a number from 57 to 183 corresponding to the amino acid sequence identified in SEQ ID NO:3. In specific embodiments, antibodies of the invention bind C terminal deletions of the TR7 polypeptide comprising, or alternatively, consisting of, amino acid residues: E-52 to S-183; E-52 to E-182; E-52 to K-181; E-52 to H-180; E-52 to V-179; E-52 to C-178; E-52 to E-177; E-52 to I-176; E-52 to D-175; E-52 to S-174; E-52 to W-173; E-52 to P-172; E-52 to T-171; E-52 to C-170; E-52 to D-169; E-52 to G-168; E-52 to V-167; E-52 to K-166; E-52 to V-165; E-52 to M-164; E-52 to G-163; E-52 to R-162; E-52 to P-161; E-52 to C-160; E-52 to G-159; E-52 to T-158; E-52 to R-157; E-52 to C-156; E-52 to K-155; E-52 to R-154; E-52 to C-153; E-52 to M-152; E-52 to E-151; E-52 to P-150; E-52 to S-149; E-52 to D-148; E-52 to E-147; E-52 to E-146; E-52 to R-145; E-52 to F-144; E-52 to T-143; E-52 to G-142; E-52 to E-141; E-52 to E-140; E-52 to C-139; E-52 to Q-138; E-52 to C-137; E-52 to V-136; E-52 to T-135; E-52 to N-134; E-52 to R-133; E-52 to T-132; E-52 to T-131; E-52 to T-130; E-52 to C-129; E-52 to P-128; E-52 to S-127; E-52 to L-126; E-52 to E-125; E-52 to V-124; E-52 to E-123; E-52 to G-122; E-52 to S-121; E-52 to D-120; E-52 to C-119; E-52 to R-118; E-52 to T-117; E-52 to C-116; E-52 to R-115; E-52 to L-114; E-52 to C-113; E-52 to F-12; E-52 to L-11; E-52 to L-110; E-52 to D-109; E-52 to N-108; E-52 to W-107; E-52 to H-106; E-52 to T-105; E-52 to S-104; E-52 to Y-103; E-52 to D-102; E-52 to Q-101; E-52 to G-100; E-52 to Y-99; E-52 to K-98; E-52 to C-97; E-52 to S-96; E-52 to I-95; E-52 to C-94; E-52 to D-93; E-52 to R-92; E-52 to G-91; E-52 to D-90; E-52 to E-89; E-52 to S-88; E-52 to I-87; E-52 to H-86; E-52 to H-85; E-52 to G-84; E-52 to P-83; E-52 to P-82; E-52 to C-81; E-52 to L-80; E-52 to G-79; E-52 to E-78; E-52 to S-77; E-52 to P-76; E-52 to S-75; E-52 to S-74; E-52 to R-73; E-52 to K-72; E-52 to Q-71; E-52 to Q-70; E-52 to P-69; E-52 to A-68; E-52 to A-67; E-52 to R-66; E-52 to Q-65; E-52 to Q-64; E-52 to P-63; E-52 to A-62; E-52 to L-61; E-52 to D-60; E-52 to Q-59; E-52 to Q-58; and/or E-52 to T-57; of the TR7 extracellular domain sequence shown in SEQ ID NO:3.

The invention also provides antibodies that bind polypeptides having one or more amino acids deleted from both the amino and the carboxyl termini of a TR7 polypeptide, which may be described generally as having residues n⁵-m⁵ and/or n⁶-m⁶ of SEQ ID NO:3, where n⁵, n⁶, m⁵, and m⁶ are integers as described above.

Also included are antibodies that bind a polypeptide consisting of a portion of the complete TR7 amino acid sequence encoded by the cDNA clone contained in ATCC Deposit No. 97920, where this portion excludes from 1 to about 78 amino acids from the amino terminus of the complete amino acid sequence encoded by the cDNA clone contained in ATCC Deposit No. 97920, or from 1 to about 233 amino acids from the carboxy terminus, or any combination of the above amino terminal and carboxy terminal deletions, of the complete amino acid sequence encoded by the cDNA clone contained in ATCC Deposit No. 97920.

Preferably, antibodies of the present invention bind the N- and C-terminal deletion mutants comprising only a portion of the extracellular domain; i.e., within residues 52-184 of SEQ ID NO:3, since any portion therein is expected to be soluble.

It will be recognized in the art that some amino acid sequence of TR7 can be varied without significant effect of the structure or function of the protein. If such differences in sequence are contemplated, it should be remembered that there will be critical areas on the protein which determine activity. Such areas will usually comprise residues which make up the ligand binding site or the death domain, or which form tertiary structures which affect these domains.

Thus, the invention further includes antibodies that bind variations of the TR7 protein which show substantial TR7 protein activity or which include regions of TR7, such as the protein portions discussed below. Such mutants include deletions, insertions, inversions, repeats, and type substitutions. Guidance concerning which amino acid changes are likely to be phenotypically silent can be found in Bowie, J. U. et al., Science 247:1306-1310 (1990).

Thus, antibodies of the present invention may bind a fragment, derivative, or analog of the polypeptide of SEQ ID NO:3, or that encoded by the cDNA in ATCC deposit 97920. Such fragments, variants or derivatives may be (i) one in which at least one or more of the amino acid residues are substituted with a conserved or non-conserved amino acid residue (preferably a conserved amino acid residue(s), and more preferably at least one but less than ten conserved amino acid residues) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fe fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Of particular interest are substitutions of charged amino acids with another charged amino acids and with neutral or negatively charged amino acids. The latter results in proteins with reduced positive charge to improve the characteristics of the TR7 protein. The prevention of aggregation is highly desirable. Aggregation of proteins not only results in a loss of activity but can also be problematic when preparing pharmaceutical formulations, because they can be immunogenic. (Pinckard et al., Clin Exp. Immunol. 2:331-340 (1967); Robbins et al., Diabetes 36:838-845 (1987); Cleland et al. Crit. Rev. Therapeutic Drug Carrier Systems 10:307-377 (1993)).

The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the antibodies of the present invention may bind a TR7 receptor that contains one or more amino acid substitutions, deletions or additions, either from natural mutations or human manipulation.

As indicated, changes are preferably of a minor nature, such as conservative amino acid substitutions that do not significantly affect the folding or activity of the protein (see Table 3 above).

In specific embodiments, the number of substitutions, additions or deletions in the amino acid sequence of SEQ ID NO:3 and/or any of the polypeptide fragments described herein (e.g., the extracellular domain) is 75, 70, 60, 50, 40, 35, 30, 25, 20, 15, 10, 9, 8, 7, 6, 5, 4, 3, 2, 1 or 30-20, 20-15, 20-10, 15-10, 10-1, 5-10, 1-5, 1-3 or 1-2.

In specific embodiments, the antibodies of the invention bind TR7 polypeptides or fragments or variants thereof (especially a fragment comprising or alternatively consisting of, the extracellular soluble domain of TR7), that contains any one or more of the following conservative mutations in TR7: M1 replaced with A, G, I, L, S, T, or V; E2 replaced with D; Q3 replaced with N; R4 replaced with H, or K; G5 replaced with A, I, L, S, T, M, or V; Q6 replaced with N; N7 replaced with Q; A8 replaced with G, I, L, S, T, M, or V; A10 replaced with G, I, L, S, T, M, or V; A11 replaced with G, I, L, S, T, M, or V; S12 replaced with A, G, I, L, T, M, or V; G13 replaced with A, I, L, S, T, M, or V; A14 replaced with G, I, L, S, T, M, or V; R15 replaced with H, or K; K16 replaced with H, or R; R17 replaced with H, or K; H18 replaced with K, or R; G19 replaced with A, I, L, S, T, M, or V; G21 replaced with A, I, L, S, T, M, or V; R23 replaced with H, or K; E24 replaced with D; A25 replaced with G, I, L, S, T, M, or V; R26 replaced with H, or K; G27 replaced with A, I, L, S, T, M, or V; A28 replaced with G, I, L, S, T, M, or V; R29 replaced with H, or K; G31 replaced with A, I, L, S, T, M, or V; R33 replaced with H, or K; V34 replaced with A, G, I, L, S, T, or M; K36 replaced with H, or R; T37 replaced with A, G, I, L, S, M, or V; L38 replaced with A, G, I, S, T, M, or V; V39 replaced with A, G, I, L, S, T, or M; L40 replaced with A, G, I, S, T, M, or V; V41 replaced with A, G, I, L, S, T, or M; V42 replaced with A, G, I, L, S, T, or M; A43 replaced with G, I, L, S, T, M, or V; A44 replaced with G, I, L, S, T, M, or V; V45 replaced with A, G, I, L, S, T, or M; L46 replaced with A, G, I, S, T, M, or V; L47 replaced with A, G, I, S, T, M, or V; L48 replaced with A, G, I, S, T, M, or V; V49 replaced with A, G, I, L, S, T, or M; S50 replaced with A, G, I, L, T, M, or V; A51 replaced with G, I, L, S, T, M, or V; E52 replaced with D; S53 replaced with A, G, I, L, T, M, or V; A54 replaced with G, I, L, S, T, M, or V; L55 replaced with A, G, I, S, T, M, or V; I56 replaced with A, G, L, S, T, M, or V; T57 replaced with A, G, I, L, S, M, or V; Q58 replaced with N; Q59 replaced with N; D60 replaced with E; L61 replaced with A, G, I, S, T, M, or V; A62 replaced with G, I, L, S, T, M, or V; Q64 replaced with N; Q65 replaced with N; R66 replaced with H, or K; A67 replaced with G, I, L, S, T, M, or V; A68 replaced with G, I, L, S, T, M, or V; Q70 replaced with N; Q71 replaced with N; K72 replaced with H, or R; R73 replaced with H, or K; S74 replaced with A, G, I, L, T, M, or V; S75 replaced with A, G, I, L, T, M, or V; S77 replaced with A, G, I, L, T, M, or V; E78 replaced with D; G79 replaced with A, I, L, S, T, M, or V; L80 replaced with A, G, I, S, T, M, or V; G84 replaced with A, I, L, S, T, M, or V; H85 replaced with K, or R; H86 replaced with K, or R; I87 replaced with A, G, L, S, T, M, or V; S88 replaced with A, G, I, L, T, M, or V; E89 replaced with D; D90 replaced with E; G91 replaced with A, I, L, S, T, M, or V; R92 replaced with H, or K; D93 replaced with E; I95 replaced with A, G, L, S, T, M, or V; S96 replaced with A, G, I, L, T, M, or V; K98 replaced with H, or R; Y99 replaced with F, or W; G100 replaced with A, I, L, S, T, M, or V; Q101 replaced with N; D102 replaced with E; Y103 replaced with F, or W; S104 replaced with A, G, I, L, T, M, or V; T105 replaced with A, G, I, L, S, M, or V; H106 replaced with K, or R; W107 replaced with F, or Y; N108 replaced with Q; D109 replaced with E; L110 replaced with A, G, I, S, T, M, or V; L111 replaced with A, G, I, S, T, M, or V; F112 replaced with W, or Y; L114 replaced with A, G, I, S, T, M, or V; R115 replaced with H, or K; T117 replaced with A, G, I, L, S, M, or V; R118 replaced with H, or K; D120 replaced with E; S121 replaced with A, G, I, L, T, M, or V; G122 replaced with A, I, L, S, T, M, or V; E123 replaced with D; V124 replaced with A, G, I, L, S, T, or M; E125 replaced with D; L126 replaced with A, G, I, S, T, M, or V; S127 replaced with A, G, I, L, T, M, or V; T130 replaced with A, G, I, L, S, M, or V; T131 replaced with A, G, I, L, S, M, or V; T132 replaced with A, G, I, L, S, M, or V; R133 replaced with H, or K; N134 replaced with Q; T135 replaced with A, G, I, L, S, M, or V; V136 replaced with A, G, I, L, S, T, or M; Q138 replaced with N; E140 replaced with D; E141 replaced with D; G142 replaced with A, I, L, S, T, M, or V; T143 replaced with A, G, I, L, S, M, or V; F144 replaced with W, or Y; R145 replaced with H, or K; E146 replaced with D; E147 replaced with D; D148 replaced with E; S149 replaced with A, G, I, L, T, M, or V; E151 replaced with D; M152 replaced with A, G, I, L, S, T, or V; R154 replaced with H, or K; K155 replaced with H, or R; R157 replaced with H, or K; T158 replaced with A, G, I, L, S, M, or V; G159 replaced with A, I, L, S, T, M, or V; R162 replaced with H, or K; G163 replaced with A, I, L, S, T, M, or V; M164 replaced with A, G, I, L, S, T, or V; V165 replaced with A, G, I, L, S, T, or M; K166 replaced with H, or R; V167 replaced with A, G, I, L, S, T, or M; G168 replaced with A, I, L, S, T, M, or V; D169 replaced with E; T171 replaced with A, G, I, L, S, M, or V; W173 replaced with F, or Y; S174 replaced with A, G, I, L, T, M, or V; D175 replaced with E; I176 replaced with A, G, I, L, S, T, M, or V; E177 replaced with D; V179 replaced with A, G, I, L, S, T, or M; H180 replaced with K, or R; K181 replaced with H, or R; E182 replaced with D; S183 replaced with A, G, I, L, T, M, or V; G184 replaced with A, I, L, S, T, M, or V; I185 replaced with A, G, L, S, T, M, or V; I186 replaced with A, G, L, S, T, M, or V; I187 replaced with A, G, L, S, T, M, or V; G188 replaced with A, I, L, S, T, M, or V; V189 replaced with A, G, I, L, S, T, or M; T190 replaced with A, G, I, L, S, M, or V; V191 replaced with A, G, I, L, S, T, or M; A192 replaced with G, I, L, S, T, M, or V; A193 replaced with G, I, L, S, T, M, or V; V194 replaced with A, G, I, L, S, T, or M; V195 replaced with A, G, I, L, S, T, or M; L196 replaced with A, G, I, S, T, M, or V; I197 replaced with A, G, L, S, T, M, or V; V198 replaced with A, G, I, L, S, T, or M; A199 replaced with G, I, L, S, T, M, or V; V200 replaced with A, G, I, L, S, T, or M; F201 replaced with W, or Y; V202 replaced with A, G, I, L, S, T, or M; K204 replaced with H, or R; S205 replaced with A, G, I, L, T, M, or V; L206 replaced with A, G, I, S, T, M, or V; L207 replaced with A, G, I, S, T, M, or V; W208 replaced with F, or Y; K209 replaced with H, or R; K210 replaced with H, or R; V211 replaced with A, G, I, L, S, T, or M; L212 replaced with A, G, I, S, T, M, or V; Y214 replaced with F, or W; L215 replaced with A, G, I, S, T, M, or V; K216 replaced with H, or R; G217 replaced with A, I, L, S, T, M, or V; I218 replaced with A, G, L, S, T, M, or V; S220 replaced with A, G, I, L, T, M, or V; G221 replaced with A, I, L, S, T, M, or V; G222 replaced with A, I, L, S, T, M, or V; G223 replaced with A, I, L, S, T, M, or V; G224 replaced with A, I, L, S, T, M, or V; D225 replaced with E; E227 replaced with D; R228 replaced with H, or K; V229 replaced with A, G, I, L, S, T, or M; D230 replaced with E; R231 replaced with H, or K; S232 replaced with A, G, I, L, T, M, or V; S233 replaced with A, G, I, L, T, M, or V; Q234 replaced with N; R235 replaced with H, or K; G237 replaced with A, I, L, S, T, M, or V; A238 replaced with G, I, L, S, T, M, or V; E239 replaced with D; D240 replaced with E; N241 replaced with Q; V242 replaced with A, G, I, L, S, T, or M; L243 replaced with A, G, I, S, T, M, or V; N244 replaced with Q; E245 replaced with D; I246 replaced with A, G, L, S, T, M, or V; V247 replaced with A, G, I, L, S, T, or M; S248 replaced with A, G, I, L, T, M, or V; I249 replaced with A, G, L, S, T, M, or V; L250 replaced with A, G, I, S, T, M, or V; Q251 replaced with N; T253 replaced with A, G, I, L, S, M, or V; Q254 replaced with N; V255 replaced with A, G, I, L, S, T, or M; E257 replaced with D; Q258 replaced with N; E259 replaced with D; M260 replaced with A, G, I, L, S, T, or V; E261 replaced with D; V262 replaced with A, G, I, L, S, T, or M; Q263 replaced with N; E264 replaced with D; A266 replaced with G, I, L, S, T, M, or V; E267 replaced with D; T269 replaced with A, G, I, L, S, M, or V; G270 replaced with A, I, L, S, T, M, or V; V271 replaced with A, G, I, L, S, T, or M; N272 replaced with Q; M273 replaced with A, G, I, L, S, T, or V; L274 replaced with A, G, I, S, T, M, or V; S275 replaced with A, G, I, L, T, M, or V; G277 replaced with A, I, L, S, T, M, or V; E278 replaced with D; S279 replaced with A, G, I, L, T, M, or V; E280 replaced with D; H281 replaced with K, or R; L282 replaced with A, G, I, S, T, M, or V; L283 replaced with A, G, I, S, T, M, or V; E284 replaced with D; A286 replaced with G, I, L, S, T, M, or V; E287 replaced with D; A288 replaced with G, I, L, S, T, M, or V; E289 replaced with D; R290 replaced with H, or K; S291 replaced with A, G, I, L, T, M, or V; Q292 replaced with N; R293 replaced with H, or K; R294 replaced with H, or K; R295 replaced with H, or K; L296 replaced with A, G, I, S, T, M, or V; L297 replaced with A, G, I, S, T, M, or V; V298 replaced with A, G, I, L, S, T, or M; A300 replaced with G, I, L, S, T, M, or V; N301 replaced with Q; E302 replaced with D; G303 replaced with A, I, L, S, T, M, or V; D304 replaced with E; T306 replaced with A, G, I, L, S, M, or V; E307 replaced with D; T308 replaced with A, G, I, L, S, M, or V; L309 replaced with A, G, I, S, T, M, or V; R310 replaced with H, or K; Q311 replaced with N; F313 replaced with W, or Y; D314 replaced with E; D315 replaced with E; F316 replaced with W, or Y; A317 replaced with G, I, L, S, T, M, or V; D318 replaced with E; L319 replaced with A, G, I, S, T, M, or V; V320 replaced with A, G, I, L, S, T, or M; F322 replaced with W, or Y; D323 replaced with E; S324 replaced with A, G, I, L, T, M, or V; W325 replaced with F, or Y; E326 replaced with D; L328 replaced with A, G, I, S, T, M, or V; M329 replaced with A, G, I, L, S, T, or V; R330 replaced with H, or K; K331 replaced with H, or R; L332 replaced with A, G, I, S, T, M, or V; G333 replaced with A, I, L, S, T, M, or V; L334 replaced with A, G, I, S, T, M, or V; M335 replaced with A, G, I, L, S, T, or V; D336 replaced with E; N337 replaced with Q; E338 replaced with D; I339 replaced with A, G, L, S, T, M, or V; K340 replaced with H, or R; V341 replaced with A, G, I, L, S, T, or M; A342 replaced with G, I, L, S, T, M, or V; K343 replaced with H, or R; A344 replaced with G, I, L, S, T, M, or V; E345 replaced with D; A346 replaced with G, L, S, T, M, or V; A347 replaced with G, I, L, S, T, M, or V; G348 replaced with A, I, L, S, T, M, or V; H349 replaced with K, or R; R350 replaced with H, or K; D351 replaced with E; T352 replaced with A, G, I, L, S, M, or V; L353 replaced with A, G, I, S, T, M, or V; Y354 replaced with F, or W; T355 replaced with A, G, I, L, S, M, or V; M356 replaced with A, G, I, L, S, T, or V; L357 replaced with A, G, I, S, T, M, or V; I358 replaced with A, G, L, S, T, M, or V; K359 replaced with H, or R; W360 replaced with F, or Y; V361 replaced with A, G, I, L, S, T, or M; N362 replaced with Q; K363 replaced with H, or R; T364 replaced with A, G, I, L, S, M, or V; G365 replaced with A, I, L, S, T, M, or V; R366 replaced with H, or K; D367 replaced with E; A368 replaced with G, I, L, S, T, M, or V; S369 replaced with A, G, I, L, T, M, or V; V370 replaced with A, G, I, L, S, T, or M; H371 replaced with K, or R; T372 replaced with A, G, I, L, S, M, or V; L373 replaced with A, G, I, S, T, M, or V; L374 replaced with A, G, I, S, T, M, or V; D375 replaced with E; A376 replaced with G, I, L, S, T, M, or V; L377 replaced with A, G, I, S, T, M, or V; E378 replaced with D; T379 replaced with A, G, I, L, S, M, or V; L380 replaced with A, G, I, S, T, M, or V; G381 replaced with A, I, L, S, T, M, or V; E382 replaced with D; R383 replaced with H, or K; L384 replaced with A, G, I, S, T, M, or V; A385 replaced with G, I, L, S, T, M, or V; K386 replaced with H, or R; Q387 replaced with N; K388 replaced with H, or R; I389 replaced with A, G, L, S, T, M, or V; E390 replaced with D; D391 replaced with E; H392 replaced with K, or R; L393 replaced with A, G, I, S, T, M, or V; L394 replaced with A, G, I, S, T, M, or V; S395 replaced with A, G, I, L, T, M, or V; S396 replaced with A, G, I, L, T, M, or V; G397 replaced with A, I, L, S, T, M, or V; K398 replaced with H, or R; F399 replaced with W, or Y; M400 replaced with A, G, I, L, S, T, or V; Y401 replaced with F, or W; L402 replaced with A, G, I, S, T, M, or V; E403 replaced with D; G404 replaced with A, I, L, S, T, M, or V; N405 replaced with Q; A406 replaced with G, I, L, S, T, M, or V; D407 replaced with E; S408 replaced with A, G, I, L, T, M, or V; A409 replaced with G, I, L, S, T, M, or V; M410 replaced with A, G, I, L, S, T, or V; and/or S411 replaced with A, G, I, L, T, M, or V of SEQ ID NO:3.

In specific embodiments, the antibodies of the invention bind TR7 polypeptides or fragments or variants thereof (especially a fragment comprising or alternatively consisting of, the extracellular soluble domain of TR7), that contains any one or more of the following non-conservative mutations in TR7: M1 replaced with D, E, H, K, R, N, Q, F, W, Y, P, or C; E2 replaced with H, K, R, A, G, I, L, S, T, M, V, N, Q, F, W, Y, P, or C; Q3 replaced with D, E, H, K, R, A, G, I, L, S, T, M, V, F, W, Y, P, or C; R4 replaced with D, E, A, G, I, L, S, T, M, V, N, Q, F, W, Y, P, or C; G5 replaced with D, E, H, K, K, R, N, Q, F, W, Y, P, or C; Q6 replaced with D, E, H, K, R, A, G, I, L, S, T, M, V, F, W, Y, P, or C; N7 replaced with D, E, H, K, R, A, G, I, L, S, T, M, V, F, W, Y, P, or C; A8 replaced with D, E, H, K, R, N, Q, F, W, Y, P, or C; P9 replaced with D, E, H, K, K, R, A, G, I, L, S, T, M, V, N, Q, F, W, Y, or C; A10 replaced with D, E, H, K, R, N, Q, F, W, Y, P, or C; A11 replaced with D, E, H, K, R, N, Q, F, W, Y, P, or C; S12 replaced with D, E, H, K, R, N, Q, F, W, Y, P, or C; G13 replaced with D, E, H, K, R, N, Q, F, W, Y, P, or C; A14 replaced with D, E, H, K, R, N, Q, F, W, Y, P, or C; R15 replaced with D, E, A, G, I, L, S, T, M, V, N, Q, F, W, Y, P, or C; K16 replaced with D, E, A, G, I, L, S, T, M, V, N, Q, F, W, Y, P, or C; R17 replaced with D, E, A, G, I, L, S, T, M, V, N, Q, F, W, Y, P, or C; H18 replaced with D, E, A, G, I, L, S, T, M, V, N, Q, F, W, Y, P, or C; G19 replaced with D, E, H, K, R, N, Q, F, W, Y, P, or C; P20 replaced with D, E, H, K, R, A, G, I, L, S, T, M, V, N, Q, F, W, Y, or C; G21 replaced with D, E, H, K, R, N, Q, F, W, Y, P, or C; P22 replaced with D, E, H, K, R, A, G, I, L, S, T, M, V, N, Q, F, W, Y, or C; R23 replaced with D, E, A, G, I, L, S, T, M, V, N, Q, F, W, Y, P, or C; E24 replaced with H, K, R, A, G, I, L, S, T, M, V, N, Q, F, W, Y, P, or C; A25 replaced with D, E, H, K, R, N, Q, F, W, Y, P, or C; R26 replaced with D, E, A, G, I, L, S, T, M, V, N, Q, F, W, Y, P, or C; G27 replaced with D, E, H, K, R, N, Q, F, W, Y, P, or C; A28 replaced with D, E, H, K, R, N, Q, F, W, Y, P, or C; R29 replaced with D, E, A, G, I, L, S, T, M, V, N, Q, F, W, Y, P, or C; P30 replaced with D, E, H, K, R, A, G, I, L, S, T, M, V, N, Q, F, W, Y, or C; G31 replaced with D, E, H, K, R, N, Q, F, W, Y, P, or C; P32 replaced with D, E, H, K, R, A, G, I, L, S, T, M, V, N, Q, F, W, Y, or C; R33 replaced with D, E, A, G, I, L, S, T, M, V, N, Q, F, W, Y, P, or C; V34 replaced with D, E, H, K, R, N, Q, F, W, Y, P, or C; P35 replaced with D, E, H, K, R, A, G, I, L, S, T, M, V, N, Q, F, W, Y, or C; K36 replaced with D, E, A, G, I, L, S, T, M, V, N, Q, F, W, Y, P, or C; T37 replaced with D, E, H, K, R, N, Q, F, W, Y, P, or C; L38 replaced with D, E, H, K, R, N, Q, F, W, Y, P, or C; V39 replaced with D, E, H, K, R, N, Q, F, W, Y, P, or C; L40 replaced with D, E, H, K, R, N, Q, F, W, Y, P, or C; V41 replaced with D, E, H, K, R, N, Q, F, W, Y, P, or C; V42 replaced with D, E, H, K, R, N, Q, F, W, Y, P, or C; A43 replaced with D, E, H, K, R, N, Q, F, W, Y, P, or C; A44 replaced with D, E, H, K, R, N, Q, F, W, Y, P, or C; V45 replaced with D, E, H, K, R, N, Q, F, W, Y, P, or C; L46 replaced with D, E, H, K, R, N, Q, F, W, Y, P, or C; L47 replaced with D, E, H, K, R, N, Q, F, W, Y, P, or C; L48 replaced with D, E, H, K, R, N, Q, F, W, Y, P, or C; V49 replaced with D, E, H, K, R, N, Q, F, W, Y, P, or C; S50 replaced with D, E, H, K, R, N, Q, F, W, Y, P, or C; A51 replaced with D, E, H, K, R, N, Q, F, W, Y, P, or C; E52 replaced with H, K, R, A, G, I, L, S, T, M, V, N, Q, F, W, Y, P, or C; S53 replaced with D, E, H, K, R, N, Q, F, W, Y, P, or C; A54 replaced with D, E, H, K, R, N, Q, F, W, Y, P, or C; L55 replaced with D, E, H, K, R, N, Q, F, W, Y, P, or C; I56 replaced with D, E, H, K, R, N, Q, F, W, Y, P, or C; T57 replaced with D, E, H, K, R, N, Q, F, W, Y, P, or C; Q58 replaced with D, E, H, K, R, A, G, I, L, S, T, M, V, F, W, Y, P, or C; Q59 replaced with D, E, H, K, R, A, G, I, L, S, T, M, V, F, W, Y, P, or C; D60 replaced with H, K, R, A, G, I, L, S, T, M, V, N, Q, F, W, Y, P, or C; L61 replaced with D, E, H, K, R, N, Q, F, W, Y, P, or C; A62 replaced with D, E, I, K, R, N, Q, F, W, Y, P, or C; P63 replaced with D, E, H, K, R, A, G, I, L, S, T, M, V, N, Q, F, W, Y, or C; Q64 replaced with D, E, H, K, R, A, G, I, L, S, T, M, V, F, W, Y, P, or C; Q65 replaced with D, E, H, K, R, A, G, I, L, S, T, M, V, F, W, Y, P, or C; R66 replaced with D, E, A, G, I, L, S, T, M, V, N, Q, F, W, Y, P, or C; A67 replaced with D, E, H, K, R, N, Q, F, W, Y, P, or C; A68 replaced with D, E, H, K, R, N, Q, F, W, Y, P, or C; P69 replaced with D, E, H, K, R, A, G, I, L, S, T, M, V, N, Q, F, W, Y, or C; Q70 replaced with D, E, H, K, R, A, G, I, L, S, T, M, V, F, W, Y, P, or C; Q71 replaced with D, E, H, K, R, A, G, I, L, S, T, M, V, F, W, Y, P, or C; K72 replaced with D, E, A, G, I, L, S, T, M, V, N, Q, F, W, Y, P, or C; R73 replaced with D, E, A, G, I, L, S, T, M, V, N, Q, F, W, Y, P, or C; S74 replaced with D, E, H, K, R, N, Q, F, W, Y, P, or C; S75 replaced with D, E, H, K, R, N, Q, F, W, Y, P, or C; P76 replaced with D, E, H, K, R, A, G, I, L, S, T, M, V, N, Q, F, W, Y, or C; S77 replaced with D, E, H, K, R, N, Q, F, W, Y, P, or C; E78 replaced with H, K, R, A, G, I, L, S, T, M, V, N, Q, F, W, Y, P, or C; G79 replaced with D, E, H, K, R, N, Q, F, W, Y, P, or C; L80 replaced with D, E, H, K, R, N, Q, F, W, Y, P, or C; C81 replaced with D, E, H, K, R, A, G, I, L, S, T, M, V, N, Q, F, W, Y, or P; P82 replaced with D, E, H, K, R, A, G, I, L, S, T, M, V, N, Q, F, W, Y, or C; P83 replaced with D, E, H, K, R, A, G, I, L, S, T, M, V, N, Q, F, W, Y, or C; G84 replaced with D, E, H, K, R, N, Q, F, W, Y, P, or C; H85 replaced with D, E, A, G, I, L, S, T, M, V, N, Q, F, W, Y, P, or C; H86 replaced with D, E, A, G, I, L, S, T, M, V, N, Q, F, W, Y, P, or C; I87 replaced with D, E, H, K, R, N, Q, F, W, Y, P, or C; S88 replaced with D, E, H, K, R, N, Q, F, W, Y, P, or C; E89 replaced with H, K, R, A, G, I, L, S, T, M, V, N, Q, F, W, Y, P, or C; D90 replaced with H, K, R, A, G, I, L, S, T, M, V, N, Q, F, W, Y, P, or C; G91 replaced with D, E, H, K, R, N, Q, F, W, Y, P, or C; R92 replaced with D, E, A, G, I, L, S, T, M, V, N, Q, F, W, Y, P, or C; D93 replaced with H, K, R, A, G, I, L, S, T, M, V, N, Q, F, W, Y, P, or C; C94 replaced with D, E, H, K, R, A, G, I, L, S, T, M, V, N, Q, F, W, Y, or P; I95 replaced with D, E, H, K, R, N, Q, F, W, Y, P, or C; S96 replaced with D, E, H, K, R, N, Q, F, W, Y, P, or C; C97 replaced with D, E, H, K, R, A, G, I, L, S, T, M, V, N, Q, F, W, Y, or P; K98 replaced with D, E, A, G, I, L, S, T, M, V, N, Q, F, W, Y, P, or C; Y99 replaced with D, E, H, K, R, N, Q, A, G, I, L, S, T, M, V, P, or C; D100 replaced with D, E, H, K, R, N, Q, F, W, Y, P, or C; Q101 replaced with D, E, H, K, R, A, G, I, L, S, T, M, V, F, W, Y, P, or C; D102 replaced with H, K, R, A, G, I, L, S, T, M, V, N, Q, F, W, Y, P, or C; Y103 replaced with D, E, H, K, R, N, Q, A, G, I, L, S, T, M, V, P, or C; S104 replaced with D, E, H, K, R, N, Q, F, W, Y, P, or C; T105 replaced with D, E, H, K, R, N, Q, F, W, Y, P, or C; H106 replaced with D, E, A, G, I, L, S, T, M, V, N, Q, F, W, Y, P, or C; W107 replaced with D, E, H, K, R, N, Q, A, G, I, L, S, T, M, V, P, or C; N108 replaced with D, E, H, K, R, A, G, I, L, S, T, M, V, F, W, Y, P, or C; D109 replaced with H, K, R, A, G, I, L, S, T, M, V, N, Q, F, W, Y, P, or C; L110 replaced with D, E, H, K, R, N, Q, F, W, Y, P, or C; L111 replaced with D, E, H, K, R, N, Q, F, W, Y, P, or C; F112 replaced with D, E, H, K, R, N, Q, A, G, I, L, S, T, M, V, P, or C; C113 replaced with D, E, H, K, R, A, G, I, L, S, T, M, V, N, Q, F, W, Y, or P; L14 replaced with D, E, H, K, R, N, Q, F, W, Y, P, or C; R115 replaced with D, E, A, G, I, L, S, T, M, V, N, Q, F, W, Y, P, or C; C116 replaced with D, E, H, K, R, A, G, I, L, S, T, M, V, N, Q, F, W, Y, or P; T117 replaced with D, E, H, K, R, N, Q, F, W, Y, P, or C; R18 replaced with D, E, A, G, I, L, S, T, M, V, N, Q, F, W, Y, P, or C; C119 replaced with D, E, H, K, K, R, A, G, I, L, S, T, M, V, N, Q, F, W, Y, or P; D120 replaced with H, K, R, A, G, I, L, S, T, M, V, N, Q, F, W, Y, P, or C; S121 replaced with D, E, H, K, R, N, Q, F, W, Y, P, or C; G122 replaced with D, E, H, K, R, N, Q, F, W, Y, P, or C; E123 replaced with H, K, R, A, G, I, L, S, T, M, V, N, Q, F, W, Y, P, or C; V124 replaced with D, E, H, K, R, N, Q, F, W, Y, P, or C; E125 replaced with H, K, R, A, G, I, L, S, T, M, V, N, Q, F, W, Y, P, or C; L126 replaced with D, E, H, K, R, N, Q, F, W, Y, P, or C; S127 replaced with D, E, H, K, R, N, Q, F, W, Y, P, or C; P128 replaced with D, E, H, K, R, A, G, I, L, S, T, M, V, N, Q, F, W, Y, or C; C129 replaced with D, E, H, K, R, A, G, I, L, S, T, M, V, N, Q, F, W, Y, or P; T130 replaced with D, E, H, K, R, N, Q, F, W, Y, P, or C; T131 replaced with D, E, H, K, R, N, Q, F, W, Y, P, or C; T132 replaced with D, E, H, K, R, N, Q, F, W, Y, P, or C; R133 replaced with D, E, A, G, I, L, S, T, M, V, N, Q, F, W, Y, P, or C; N134 replaced with D, E, H, K, R, A, G, I, L, S, T, V, F, W, Y, P, or C; T135 replaced with D, E, H, K, R, N, Q, F, W, Y, P, or C; V136 replaced with D, E, H, K, R, N, Q, F, W, Y, P, or C; C137 replaced with D, E, H, K, R, A, G, I, L, S, T, M, V, N, Q, F, W, Y, or P; Q138 replaced with D, E, H, K, R, A, G, I, L, S, T, M, V, F, W, Y, P, or C; C139 replaced with D, E, H, K, R, A, G, I, L, S, T, M, V, N, Q, F, W, Y, or P; E140 replaced with H, K, R, A, G, I, L, S, T, M, V, N, Q, F, W, Y, P, or C; E141 replaced with H, K, F, A, G, I, L, S, T, M, V, N, Q, F, W, Y, P, or C; G142 replaced with D, E, H, K, R, N, Q, F, W, Y, P, or C; T143 replaced with D, E, H, K, R, N, Q, F, W, Y, P, or C; F144 replaced with D, E, H, K, R, N, Q, A, G, I, L, S, T, M, V, P, or C; R145 replaced with D, E, A, G, I, L, S, T, M, V, N, Q, F, W, Y, P, or C; E146 replaced with H, K, R, A, G, I, L, S, T, M, V, N, Q, F, W, Y, P, or C; E147 replaced with H, K, R, A, G, I, L, S, T, M, V, N, Q, F, W, Y, P, or C; D148 replaced with H, K, R, A, G, I, L, S, T, M, V, N, Q, F, W, Y, P, or C; S149 replaced with D, E, H, K, R, N, Q, F, W, Y, P, or C; P150 replaced with D, E, H, K, R, A, G, I, L, S, T, M, V, N, Q, F, W, Y, or C; E151 replaced with H, K, R, A, G, I, L, S, T, M, V, N, Q, F, W, Y, P, or C; M152 replaced with D, E, H, K, R, N, Q, F, W, Y, P, or C; C153 replaced with D, E, H, K, R, A, G, I, L, S, T, M, V, N, Q, F, W, Y, or P; R154 replaced with D, E, A, G, I, L, S, T, M, V, N, Q, F, W, Y, P, or C; K155 replaced with D, E, A, G, I, L, S, T, M, V, N, Q, F, W, Y, P, or C; C156 replaced with D, E, H, K, R, A, G, I, L, S, T, M, V, N, Q, F, W, Y, or P; R157 replaced with D, E, A, G, I, L, S, T, M, V, N, Q, F, W, Y, P, or C; T158 replaced with D, E, H, K, R, N, Q, F, W, Y, P, or C; D159 replaced with D, E, H, K, R, N, Q, F, W, Y, P, or C; C160 replaced with D, E, H, K, R, A, G, I, L, S, T, M, V, N, Q, F, W, Y, or P; P161 replaced with D, E, H, K, R, A, G, I, L, S, T, M, V, N, Q, F, W, Y, or C; R162 replaced with D, E, A, G, I, L, S, T, M, V, N, Q, F, W, Y, P, or C; G163 replaced with D, E, H, K, R, N, Q, F, W, Y, P, or C; M164 replaced with D, E, H, K, R, N, Q, F, W, Y, P, or C; V165 replaced with D, E, H, K, R, N, Q, F, W, Y, P, or C; K166 replaced with D, E, A, G, I, L, S, T, M, V, N, Q, F, W, Y, P, or C; V167 replaced with D, E, H, K, R, N, Q, F, W, Y, P, or C; G168 replaced with D, E, H, K, R, N, Q, F, W, Y, P, or C; D169 replaced with H, K, R, A, G, I, L, S, T, M, V, N, Q, F, W, Y, P, or C; C170 replaced with D, E, H, K, R, A, G, I, L, S, T, M, V, N, Q, F, W, Y, or P; T171 replaced with D, E, H, K, R, N, Q, F, W, Y, P, or C; P172 replaced with D, E, H, K, R, A, G, I, L, S, T, M, V, N, Q, F, W, Y, or C; W173 replaced with D, E, H, K, R, N, Q, A, G, I, L, S, T, M, V, P, or C; S174 replaced with D, E, H, K, R, N, Q, F, W, Y, P, or C; D175 replaced with H, K, R, A, G, I, L, S, T, M, V, N, Q, F, W, Y, P, or C; I176 replaced with D, E, H, K, R, N, Q, F, W, Y, P, or C; E177 replaced with H, K, R, A, G, I, L, S, T, M, V, N, Q, F, W, Y, P, or C; C178 replaced with D, E, H, K, R, A, G, I, L, S, T, M, V, N, Q, F, W, Y, or P; V179 replaced with D, E, H, K, R, N, Q, F, W, Y, P, or C; H180 replaced with D, E, A, G, I, L, S, T, M, V, N, Q, F, W, Y, P, or C; K181 replaced with D, E, A, G, I, L, S, T, M, V, N, Q, F, W, Y, P, or C; E182 replaced with H, K, R, A, G, I, L, S, T, M, V, N, Q, F, W, Y, P, or C; S183 replaced with D, E, H, K, R, N, Q, F, W, Y, P, or C; G184 replaced with D, E, H, K, R, N, Q, F, W, Y, P, or C; I185 replaced with D, E, H, K, R, N, Q, F, W, Y, P, or C; I186 replaced with D, E, H, K, R, N, Q, F, W, Y, P, or C; I187 replaced with D, E, H, K, R, N, Q, F, W, Y, P, or C; G188 replaced with D, E, H, K, R, N, Q, F, W, Y, P, or C; V189 replaced with D, E, H, K, R, N, Q, F, W, Y, P, or C; T190 replaced with D, E, H, K, R, N, Q, F, W, Y, P, or C; V191 replaced with D, E, H, K, R, N, Q, F, W, Y, P, or C; A192 replaced with D, E, H, K, R, N, Q, F, W, Y, P, or C; A193 replaced with D, E, H, K, R, N, Q, F, W, Y, P, or C; V194 replaced with D, E, H, K, R, N, Q, F, W, Y, P, or C; V195 replaced with D, E, H, K, R, N, Q, F, W, Y, P, or C; L196 replaced with D, E, H, K, R, N, Q, F, W, Y, P, or C; I197 replaced with D, E, H, K, R, N, Q, F, W, Y, P, or C; V198 replaced with D, E, H, K, R, N, Q, F, W, Y, P, or C; A199 replaced with D, E, H, K, R, N, Q, F, W, Y, P, or C; V200 replaced with D, E, H, K, R, N, Q, F, W, Y, P, or C; F201 replaced with D, E, H, K, R, N, Q, A, G, I, L, S, T, M, V, P, or C; V202 replaced with D, E, H, K, R, N, Q, F, W, Y, P, or C; C203 replaced with D, E, H, K, R, A, G, I, L, S, T, M, V, N, Q, F, W, Y, or P; K204 replaced with D, E, A, G, I, L, S, T, M, V, N, Q, F, W, Y, P, or C; S205 replaced with D, E, H, K, E, N, Q, F, W, Y, P, or C; L206 replaced with D, E, H, K, R, N, Q, F, W, Y, P, or C; L207 replaced with D, E, H, K, R, N, Q, F, W, Y, P, or C; W208 replaced with D, E, H, K, R, N, Q, A, G, I, L, S, T, M, V, P, or C; K209 replaced with D, E, A, G, I, L, S, T, M, V, N, Q, F, W, Y, P, or C; K210 replaced with D, E, A, G, I, L, S, T, M, V, N, Q, F, W, Y, P, or C; V211 replaced with D, E, H, K, R, N, Q, F, W, Y, P, or C; L212 replaced with D, E, H, K, R, N, Q, F, W, Y, P, or C; P213 replaced with D, E, H, K, R, A, G, I, L, S, T, M, V, N, Q, F, W, Y, or C; Y214 replaced with D, E, H, K, R, N, Q, A, G, I, L, S, T, M, V, P, or C; L215 replaced with D, E, H, K, R, N, Q, F, W, Y, P, or C; K216 replaced with D, E, A, G, I, L, S, T, M, V, N, Q, F, W, Y, P, or C; G217 replaced with D, E, H, K, R, N, Q, F, W, Y, P, or C; I218 replaced with D, E, H, K, R, N, Q, F, W, Y, P, or C; C219 replaced with D, E, H, K, R, A, G, I, L, S, T, M, V, N, Q, F, W, Y, or P; S220 replaced with D, E, H, K, R, N, Q, F, W, Y, P, or C; G221 replaced with D, E, H, K, R, N, Q, F, W, Y, P, or C; G222 replaced with D, E, H, K, R, N, Q, F, W, Y, P, or C; G223 replaced with D, E, H, K, R, N, Q, F, W, Y, P, or C; G224 replaced with D, E, H, K, R, N, Q, F, W, Y, P, or C; D225 replaced with H, K, R, A, G, I, L, S, T, M, V, N, Q, F, W, Y, P, or C; P226 replaced with D, E, H, K, R, A, G, I, L, S, T, M, V, N, Q, F, W, Y, or C; E227 replaced with H, K, I, A, G, I, L, S, T, M, V, N, Q, F, W, Y, P, or C; R228 replaced with D, E, A, G, I, L, S, T, M, V, N, Q, F, W, Y, P, or C; V229 replaced with D, E, H, K, R, N, Q, F, W, Y, P, or C; D230 replaced with H, K, R, A, G, I, L, S, T, M, V, N, Q, F, W, Y, P, or C; R231 replaced with D, E, A, G, I, L, S, T, M, V, N, Q, F, W, Y, P, or C; S232 replaced with D, E, H, K, R, N, Q, F, W, Y, P, or C; S233 replaced with D, E, H, K, R, N, Q, F, W, Y, P, or C; Q234 replaced with D, E, H, K, R, A, G, I, L, S, T, M, V, F, W, Y, P, or C; R235 replaced with D, E, A, G, I, L, S, T, M, V, N, Q, F, W, Y, P, or C; P236 replaced with D, E, H, K, R, A, G, I, L, S, T, M, V, N, Q, F, W, Y, or C; G237 replaced with D, E, H, K, R, N, Q, F, W, Y, P, or C; A238 replaced with D, E, H, K, R, N, Q, F, W, Y, P, or C; E239 replaced with H, K, R, A, G, I, L, S, T, M, V, N, Q, F, W, Y, P, or C; D240 replaced with H, K, R, A, G, I, L, S, T, M, V, N, Q, F, W, Y, P, or C; N241 replaced with D, E, H, K, R, A, G, I, L, S, T, M, V, F, W, Y, P, or C; V242 replaced with D, E, H, K, R, N, Q, F, W, Y, P, or C; L243 replaced with D, E, H, K, R, N, Q, F, W, Y, P, or C; N244 replaced with D, E, H, K, R, A, G, I, L, S, T, M, V, F, W, Y, P, or C; E245 replaced with H, K, R, A, G, I, L, S, T, M, V, N, Q, F, W, Y, P, or C; I246 replaced with D, E, H, K, R, N, Q, F, W, Y, P, or C; V247 replaced with D, E, H, K, R, N, Q, F, W, Y, P, or C; S248 replaced with D, E, H, K, R, N, Q, F, W, Y, P, or C; I249 replaced with D, E, H, K, R, N, Q, F, W, Y, P, or C; L250 replaced with D, E, H, K, R, N, Q, F, W, Y, P, or C; Q251 replaced with D, E, H, K, R, A, G, I, L, S, T, M, V, F, W, Y, P, or C; P252 replaced with D, E, H, K, R, A, G, I, L, S, T, M, V, N, Q, F, W, Y, or C; T253 replaced with D, E, H, K, R, N, Q, F, W, Y, P, or C; Q254 replaced with D, E, H, K, R, A, G, I, L, S, T, M, V, F, W, Y, P, or C; V255 replaced with D, E, H, K, R, N, Q, F, W, Y, P, or C; P256 replaced with D, E, H, K, R, A, G, I, L, S, T, M, V, N, Q, F, W, Y, or C; E257 replaced with H, K, R, A, G, I, L, S, T, M, V, N, Q, F, W, Y, P, or C; Q258 replaced with D, E, H, K, R, A, G, I, L, S, T, M, V, F, W, Y, P, or C; E259 replaced with H, K, R, A, G, I, L, S, T, M, V, N, Q, F, W, Y, P, or C; M260 replaced with D, E, H, K, R, N, Q, F, W, Y, P, or C; E261 replaced with H, K, R, A, G, I, L, S, T, M, V, N, Q, F, W, Y, P, or C; V262 replaced with D, E, H, K, R, N, Q, F, W, Y, P, or C; Q263 replaced with D, E, H, K, R, A, G, I, L, S, T, M, V, F, W, Y, P, or C; E264 replaced with H, K, R, A, G, I, L, S, T, M, V, N, Q, F, W, Y, P, or C; P265 replaced with D, E, H, K, R, A, G, I, L, S, T, M, V, N, Q, F, W, Y, or C; A266 replaced with D, E, H, K, R, N, Q, F, W, Y, P, or C; E267 replaced with H, K, R, A, G, I, L, S, T, M, V, N, Q, F, W, Y, P, or C; P268 replaced with D, E, H, K, R, A, G, I, L, S, T, M, V, N, Q, F, W, Y, or C; T269 replaced with D, E, H, K, R, N, Q, F, W, Y, P, or C; G270 replaced with D, E, H, K, R, N, Q, F, W, Y, P, or C; V271 replaced with D, E, H, K, R, N, Q, F, W, Y, P, or C; N272 replaced with D, E, H, K, R, A, G, I, L, S, T, M, V, F, W, Y, P, or C; M273 replaced with D, E, H, K, R, N, Q, F, W, Y, P, or C; L274 replaced with D, E, H, K, R, N, Q, F, W, Y, P, or C; S275 replaced with D, E, H, K, R, N, Q, F, W, Y, P, or C; P276 replaced with D, E, H, K, R, A, G, I, L, S, T, M, V, N, Q, F, W, Y, or C; G277 replaced with D, E, H, K, R, N, Q, F, W, Y, P, or C; E278 replaced with H, K, R, A, G, I, L, S, T, M, V, N, Q, F, W, Y, P, or C; S279 replaced with D, E, H, K, R, N, Q, F, W, Y, P, or C; E280 replaced with H, K, R, A, G, I, L, S, T, M, V, N, Q, F, W, Y, P, or C; H281 replaced with D, E, A, G, I, L, S, T, M, V, N, Q, F, W, Y, P, or C; L282 replaced with D, E, H, K, R, N, Q, F, W, Y, P, or C; L283 replaced with D, E, H, K, R, N, Q, F, W, Y, P, or C; E284 replaced with H, K, R, A, G, I, L, S, T, M, V, N, Q, F, W, Y, P, or C; P285 replaced with D, E, H, K, R, A, G, I, L, S, T, M, V, N, Q, F, W, Y, or C; A286 replaced with D, E, H, K, R, N, Q, F, W, Y, P, or C; E287 replaced with H, K, R, A, G, I, L, S, T, M, V, N, Q, F, W, Y, P, or C; A288 replaced with D, E, H, K, R, N, Q, F, W, Y, P, or C; E289 replaced with H, K, R, A, G, I, L, S, T, M, V, N, Q, F, W, Y, P, or C; R290 replaced with D, E, A, G, I, L, S, T, M, V, N, Q, F, W, Y, P, or C; S291 replaced with D, E, H, K, R, N, Q, F, W, Y, P, or C; Q292 replaced with D, E, H, K, R, A, G, I, L, S, T, M, V, F, W, Y, P, or C; R293 replaced with D, E, A, G, I, L, S, T, M, V, N, Q, F, W, Y, P, or C; R294 replaced with D, E, A, G, I, L, S, T, M, V, N, Q, F, W, Y, P, or C; R295 replaced with D, E, A, G, I, L, S, T, M, V, N, Q, F, W, Y, P, or C; L296 replaced with D, E, H, K, R, N, Q, F, W, Y, P, or C; L297 replaced with D, E, H, K, R, N, Q, F, W, Y, P, or C; V298 replaced with D, E, H, K, R, N, Q, F, W, Y, P, or C; P299 replaced with D, E, H, K, R, A, G, I, L, S, T, M, V, N, Q, F, W, Y, or C; A300 replaced with D, E, H, K, R, N, Q, F, W, Y, P, or C; N301 replaced with D, E, H, K, R, A, G, I, L, S, T, M, V, F, W, Y, P, or C; E302 replaced with H, K, R, A, G, I, L, S, T, M, V, N, Q, F, W, Y, P, or C; G303 replaced with D, E, H, K, R, N, Q, F, W, Y, P, or C; D304 replaced with H, K, R, A, G, I, L, S, T, M, V, N, Q, F, W, Y, P, or C; P305 replaced with D, E, H, K, R, A, G, I, L, S, T, M, V, N, Q, F, W, Y, or C; T306 replaced with D, E, H, K, R, N, Q, F, W, Y, P, or C; E307 replaced with H, K, R, A, G, I, L, S, T, M, V, N, Q, F, W, Y, P, or C; T308 replaced with D, E, K, K, R, N, Q, F, W, Y, P, or C; L309 replaced with D, E, H, K, R, N, Q, F, W, Y, P, or C; R310 replaced with D, E, A, G, I, L, S, T, M, V, N, Q, F, W, Y, P, or C; Q311 replaced with D, E, H, K, R, A, G, I, L, S, T, M, V, F, W, Y, P, or C; C312 replaced with D, E, H, K, R, A, G, I, L, S, T, M, V, N, Q, F, W, Y, or P; F313 replaced with D, E, H, K, R, N, Q, A, G, I, L, S, T, M, V, P, or C; D314 replaced with H, K, R, A, G, I, L, S, T, M, V, N, Q, F, W, Y, P, or C; D315 replaced with H, K, R, A, G, I, L, S, T, M, V, N, Q, F, W, Y, P, or C; F316 replaced with D, E, H, K, R, N, Q, A, G, I, L, S, T, M, V, P, or C; A317 replaced with D, E, H, K, R, N, Q, F, W, Y, P, or C; D318 replaced with H, K, R, A, G, I, L, S, T, M, V, N, Q, F, W, Y, P, or C; L319 replaced with D, E, H, K, R, N, Q, F, W, Y, P, or C; V320 replaced with D, E, H, K, R, N, Q, F, W, Y, P, or C; P321 replaced with D, E, K, K, R, A, G, I, L, S, T, M, V, N, Q, F, W, Y, or C; F322 replaced with D, E, H, K, R, N, Q, A, G, I, L, S, T, M, V, P, or C; D323 replaced with H, K, R, A, G, I, L, S, T, M, V, N, Q, F, W, Y, P, or C; S324 replaced with D, E, H, K, R, N, Q, F, W, Y, P, or C; W325 replaced with D, E, H, K, R, N, Q, A, G, I, L, S, T, M, V, P, or C; E326 replaced with H, K, R, A, G, I, L, S, T, M, V, N, Q, F, W, Y, P, or C; P327 replaced with D, E, H, K, R, A, G, I, L, S, T, M, V, N, Q, F, W, Y, or C; L328 replaced with D, E, H, K, R, N, Q, F, W, Y, P, or C; M329 replaced with D, E, H, K, R, N, Q, F, W, Y, P, or C; R330 replaced with D, E, A, G, I, L, S, T, M, V, N, Q, F, W, Y, P, or C; K331 replaced with D, E, A, G, I, L, S, T, M, V, N, Q, F, W, Y, P, or C; L332 replaced with D, E, H, K, R, N, Q, F, W, Y, P, or C; G333 replaced with D, E, H, K, R, N, Q, F, W, Y, P, or C; L334 replaced with D, E, H, K, R, N, Q, F, W, Y, P, or C; M335 replaced with D, E, H, K, R, N, Q, F, W, Y, P, or C; D336 replaced with H, K, R, A, G, I, L, S, T, M, V, N, Q, F, W, Y, P, or C; N337 replaced with D, E, H, K, R, A, G, I, L, S, T, M, V, F, W, Y, P, or C; E338 replaced with H, K, R, A, G, I, L, S, T, M, V, N, Q, F, W, Y, P, or C; I339 replaced with D, E, H, K, R, N, Q, F, W, Y, P, or C; K340 replaced with D, E, A, G, I, L, S, T, M, V, N, Q, F, W, Y, P, or C; V341 replaced with D, E, H, K, R, N, Q, F, W, Y, P, or C; A342 replaced with D, E, H, K, R, N, Q, F, W, Y, P, or C; K343 replaced with D, E, A, G, I, L, S, T, M, V, N, Q, F, W, Y, P, or C; A344 replaced with D, E, H, K, R, N, Q, F, W, Y, P, or C; E345 replaced with H, K, R, A, G, I, L, S, T, M, V, N, Q, F, W, Y, P, or C; A346 replaced with D, E, H, K, R, N, Q, F, W, Y, P, or C; A347 replaced with D, E, H, K, R, N, Q, F, W, Y, P, or C; G348 replaced with D, E, H, K, R, N, Q, F, W, Y, P, or C; H349 replaced with D, E, A, G, I, L, S, T, M, V, N, Q, F, W, Y, P, or C; R350 replaced with D, E, A, G, I, L, S, T, M, V, N, Q, F, W, Y, P, or C; D351 replaced with H, K, R, A, G, I, L, S, T, M, V, N, Q, F, W, Y, P, or C; T352 replaced with D, E, H, K, R, N, Q, F, W, Y, P, or C; L353 replaced with D, E, H, K, R, N, Q, F, W, Y, P, or C; Y354 replaced with D, E, H, K, R, N, Q, A, G, I, L, S, T, M, V, P, or C; T355 replaced with D, E, H, K, R, N, Q, F, W, Y, P, or C; M356 replaced with D, E, H, K, R, N, Q, F, W, Y, P, or C; L357 replaced with D, E, H, K, R, N, Q, F, W, Y, P, or C; I358 replaced with D, E, H, K, R, N, Q, F, W, Y, P, or C; K359 replaced with D, E, A, G, I, L, S, T, M, V, N, Q, F, W, Y, P, or C; W360 replaced with D, E, H, K, R, N, Q, A, G, I, L, S, T, M, V, P, or C; V361 replaced with D, E, H, K, R, N, Q, F, W, Y, P, or C; N362 replaced with D, E, H, K, R, A, G, I, L, S, T, M, V, F, W, Y, P, or C; K363 replaced with D, E, A, G, I, L, S, T, M, V, N, Q, F, W, Y, P, or C; T364 replaced with D, E, H, K, R, N, Q, F, W, Y, P, or C; G365 replaced with D, E, H, K, R, N, Q, F, W, Y, P, or C; R366 replaced with D, E, A, G, I, L, S, T, M, V, N, Q, F, W, Y, P, or C; D367 replaced with H, K, R, A, G, I, L, S, T, M, V, N, Q, F, W, Y, P, or C; A368 replaced with D, E, H, K, R, N, Q, F, W, Y, P, or C; S369 replaced with D, E, H, K, R, N, Q, F, W, Y, P, or C; V370 replaced with D, E, H, K, R, N, Q, F, W, Y, P, or C; H371 replaced with D, E, A, G, I, L, S, T, M, V, N, Q, F, W, Y, P, or C; T372 replaced with D, E, H, K, R, N, Q, F, W, Y, P, or C; L373 replaced with D, E, H, K, R, N, Q, F, W, Y, P, or C; L374 replaced with D, E, H, K, R, N, Q, F, W, Y, P, or C; D375 replaced with H, K, R, A, G, I, L, S, T, M, V, N, Q, F, W, Y, P, or C; A376 replaced with D, E, H, K, R, N, Q, F, W, Y, P, or C; L377 replaced with D, E, H, K, R, N, Q, F, W, Y, P, or C; E378 replaced with H, K, R, A, G, I, L, S, T, M, V, N, Q, F, W, Y, P, or C; T379 replaced with D, E, H, K, R, N, Q, F, W, Y, P, or C; L380 replaced with D, E, H, K, R, N, Q, F, W, Y, P, or C; G381 replaced with D, E, H, K, R, N, Q, F, W, Y, P, or C; E382 replaced with H, K, R, A, G, I, L, S, T, M, V, N, Q, F, W, Y, P, or C; R383 replaced with D, E, A, G, I, L, S, T, M, V, N, Q, F, W, Y, P, or C; L384 replaced with D, E, H, K, R, N, Q, F, W, Y, P, or C; A385 replaced with D, E, H, K, R, N, Q, F, W, Y, P, or C; K386 replaced with D, E, A, G, I, L, S, T, M, V, N, Q, F, W, Y, P, or C; Q387 replaced with D, E, H, K, R, A, G, I, L, S, T, M, V, F, W, Y, P, or C; K388 replaced with D, E, A, G, I, L, S, T, M, V, N, Q, F, W, Y, P, or C; I389 replaced with D, E, H, K, R, N, Q, F, W, Y, P, or C; E390 replaced with H, K, R, A, G, I, L, S, T, M, V, N, Q, F, W, Y, P, or C; D391 replaced with H, K, R, A, G, I, L, S, T, M, V, N, Q, F, W, Y, P, or C; H392 replaced with D, E, A, G, I, L, S, T, M, V, N, Q, F, W, Y, P, or C; L393 replaced with D, E, H, K, R, N, Q, F, W, Y, P, or C; L394 replaced with D, E, H, K, R, N, Q, F, W, Y, P, or C; S395 replaced with D, E, H, K, R, N, Q, F, W, Y, P, or C; S396 replaced with D, E, H, K, R, N, Q, F, W, Y, P, or C; G397 replaced with D, E, H, K, R, N, Q, F, W, Y, P, or C; K398 replaced with D, E, A, G, I, L, S, T, M, V, N, Q, F, W, Y, P, or C; F399 replaced with D, E, H, K, R, N, Q, A, G, I, L, S, T, M, V, P, or C; M400 replaced with D, E, H, K, R, N, Q, F, W, Y, P, or C; Y401 replaced with D, E, H, K, R, N, Q, A, G, I, L, S, T, M, V, P, or C; L402 replaced with D, E, H, K, R, N, Q, F, W, Y, P, or C; E403 replaced with H, K, R, A, G, I, L, S, T, M, V, N, Q, F, W, Y, P, or C; G404 replaced with D, E, H, K, R, N, Q, F, W, Y, P, or C; N405 replaced with D, E, H, K, R, A, G, I, L, S, T, M, V, F, W, Y, P, or C; A406 replaced with D, E, H, K, R, N, Q, F, W, Y, P, or C; D407 replaced with H, K, R, A, G, I, L, S, T, M, V, N, Q, F, W, Y, P, or C; S408 replaced with D, E, H, K, R, N, Q, F, W, Y, P, or C; A409 replaced with D, E, H, K, R, N, Q, F, W, Y, P, or C; M410 replaced with D, E, H, K, R, N, Q, F, W, Y, P, or C; and/or S411 replaced with D, E, H, K, R, N, Q, F, W, Y, P, or C of SEQ ID NO:3.

Amino acids in the TR7 protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro, or in vitro proliferative activity. Sites that are critical for ligand-receptor binding can also be determined by structural analysis such as crystallization, nuclear magnetic resonance or photoaffinity labeling (Smith et al., J. Mol. Biol 224:899-904 (1992) and de Vos et al. Science 255:306-312 (1992)). In preferred embodiments, antibodies of the present invention bind regions of TR7 that are essential for TR7 function. In other preferred embodiments, antibodies of the present invention bind regions of TR7 that are essential for TR7 function and inhibit or abolish TR7 function. In other preferred embodiments, antibodies of the present invention bind regions of TR7 that are essential for TR7 function and enhance TR7 function.

Additionally, protein engineering may be employed to improve or alter the characteristics of TR7 polypeptides. Recombinant DNA technology known to those skilled in the art can be used to create novel mutant proteins or polypeptides including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Antibodies of the present invention may bind such modified TR7 polypeptides.

Non-naturally occurring TR7 variants that may be bound by the antibodies of the invention may be produced using art-known mutagenesis techniques, which include, but are not limited to oligonucleotide mediated mutagenesis, alanine scanning, PCR mutagenesis, site directed mutagenesis (see e.g., Carter et al., Nucl. Acids Res. 13:4331 (1986); and Zoller et al., Nucl. Acids Res. 10:6487 (1982)), cassette mutagenesis (see e.g., Wells et al., Gene 34:315 (1985)), restriction selection mutagenesis (see e.g., Wells et al., Philos. Trans. R. Soc. London SerA 317:415 (1986)).

Thus, the invention also encompasses antibodies that bind TR7 derivatives and analogs that have one or more amino acid residues deleted, added, or substituted to generate TR7 polypeptides that are better suited for expression, scale up, etc., in the host cells chosen. For example, cysteine residues can be deleted or substituted with another amino acid residue in order to eliminate disulfide bridges; N-linked glycosylation sites can be altered or eliminated to achieve, for example, expression of a homogeneous product that is more easily recovered and purified from yeast hosts which are known to hyperglycosylate N-linked sites. To this end, a variety of amino acid substitutions at one or both of the first or third amino acid positions on any one or more of the glycosylation recognitions sequences in the TR7 polypeptides, and/or an amino acid deletion at the second position of any one or more such recognition sequences will prevent glycosylation of the TR7 at the modified tripeptide sequence (see, e.g., Miyajimo et al., EMBO J 5(6):1193-1197). Additionally, one or more of the amino acid residues of TR7 polypeptides (e.g., arginine and lysine residues) may be deleted or substituted with another residue to eliminate undesired processing by proteases such as, for example, furins or kexins.

The antibodies of the present invention also include antibodies that bind a polypeptide comprising, or alternatively, consisting of the polypeptide encoded by the deposited cDNA (the deposit having ATCC Accession Number 97920) including the leader; the mature polypeptide encoded by the deposited the cDNA minus the leader (i.e., the mature protein); a polypeptide comprising or alternatively, consisting of, amino acids about 1 to about 411 in SEQ ID NO:3; a polypeptide comprising or alternatively, consisting of, amino acids about 2 to about 411 in SEQ ID NO:3; a polypeptide comprising or alternatively, consisting of, amino acids about 52 to about 411 in SEQ ID NO:3; a polypeptide comprising or alternatively, consisting of, the TR7 extracellular domain; a polypeptide comprising or alternatively, consisting of, the TR7 cysteine rich domain; a polypeptide comprising or alternatively, consisting of, the TR7 transmembrane domain; a polypeptide comprising or alternatively, consisting of, the TR7 intracellular domain; a polypeptide comprising or alternatively, consisting of, the extracellular and intracellular domains with all or part of the transmembrane domain deleted; and a polypeptide comprising or alternatively, consisting of, the TR7 death domain; as well as polypeptides which are at least 80% identical, more preferably at least 90% or 95% identical, still more preferably at least 96%, 97%, 98%, or 99% identical to the polypeptides described above, and also include portions of such polypeptides with at least 30 amino acids and more preferably at least 50 amino acids.

By a polypeptide having an amino acid sequence at least, for example, 95% “identical” to a reference amino acid sequence of a TR7 polypeptide is intended that the amino acid sequence of the polypeptide is identical to the reference sequence except that the polypeptide sequence may include up to five amino acid alterations per each 100 amino acids of the reference amino acid of the TR7 polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

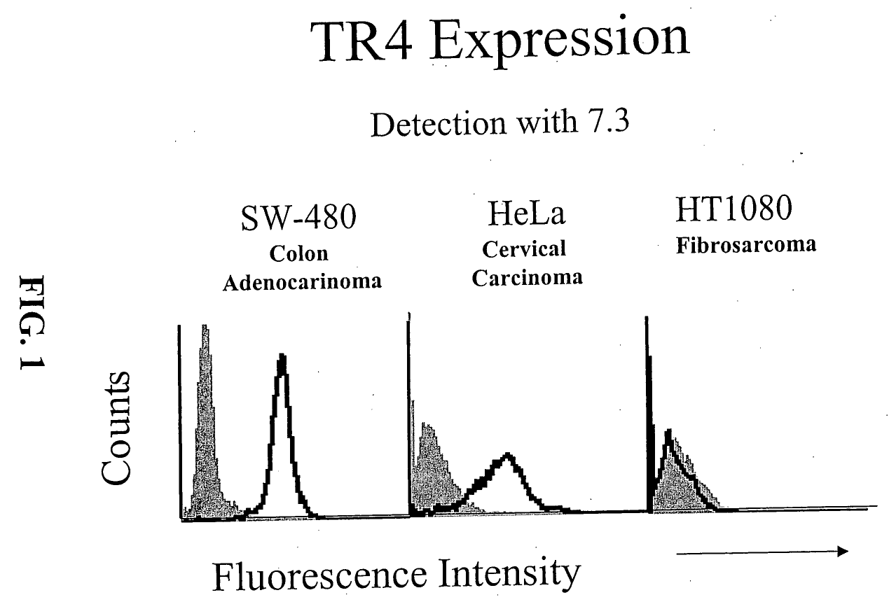

As a practical matter, whether any particular polypeptide is at least 90%, 95%, 96%, 97%, 98% or 99% identical to, for instance, the amino acid sequence shown in FIGS. 1A-B (SEQ ID NO:3), the amino acid sequence encoded by deposited cDNA clones, or fragments thereof, can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of this embodiment.

The polypeptide of the present invention could be used as a molecular weight marker on SDS-PAGE gels or on molecular sieve gel filtration columns and as a source for generating antibodies that bind the TR7 polypeptides, using methods well known to those of skill in the art.

The present application is also directed to antibodies that bind proteins containing polypeptides at least 90%, 95%, 96%, 97%, 98% or 99% identical to the TR7 polypeptide sequence set forth herein as n⁵-m⁵, and/or n⁶-m⁶. In preferred embodiments, the application is directed to antibodies that bind proteins containing polypeptides at least 90%, 95%, 96%, 97%, 98% or 99% identical to polypeptides having the amino acid sequence of the specific TR7 N- and C-terminal deletions recited herein.

In certain preferred embodiments, antibodies of the invention bind TR7 proteins of the invention comprise fusion proteins as described above wherein the TR7 polypeptides are those described as n⁵-m⁵, and n⁶-m⁶, herein.

TR10

In certain embodiments of the present invention, the antibodies of the present invention bind TR10 polypeptide, or fragments or variants thereof. The following section describes the TR10 polypeptides, fragments and variants that may be bound by the antibodies of the invention in more detail. The TR10 polypeptides, fragments and variants which may be bound by the antibodies of the invention are also described in, for example, International Publication Numbers WO98/54202 and WO00/73321 which are herein incorporated by reference in their entireties.

In certain embodiments, the antibodies of the present invention immunospecifically bind TR10 polypeptide. An antibody that immunospecifically binds TR10 may, in some embodiments, bind fragments, variants (including species orthologs of TR10), multimers or modified forms of TR10. For example, an antibody immunospecific for TR10 may bind the TR10 moiety of a fusion protein comprising all or a portion of TR10.

TR10 proteins may be found as monomers or multimers (i.e., dimers, trimers, tetramers, and higher multimers). Accordingly, the present invention relates to antibodies that bind TR10 proteins found as monomers or as part of multimers. In specific embodiments, antibodies of the invention bind TR10 monomers, dimers, trimers or tetramers. In additional embodiments, antibodies of the invention bind at least dimers, at least trimers, or at least tetramers containing one or more TR10 polypeptides.

Antibodies of the invention may bind TR10 homomers or heteromers. As used herein, the term homomer, refers to a multimer containing only TR10 proteins of the invention (including TR10 fragments, variants, and fusion proteins, as described herein). These homomers may contain TR10 proteins having identical or different polypeptide sequences. In a specific embodiment, a homomer of the invention is a multimer containing only TR10 proteins having an identical polypeptide sequence. In another specific embodiment, antibodies of the invention bind TR10 homomers containing TR10 proteins having different polypeptide sequences. In specific embodiments, antibodies of the invention bind a TR10 homodimer (e.g., containing TR10 proteins having identical or different polypeptide sequences) or a homotrimer (e.g., containing TR10 proteins having identical or different polypeptide sequences). In additional embodiments, antibodies of the invention bind at least a homodimer, at least a homotrimer, or at least a homotetramer of TR10.

As used herein, the term heteromer refers to a multimer containing heterologous proteins (i.e., proteins containing polypeptide sequences that do not correspond to a polypeptide sequences encoded by the TR10 gene) in addition to the TR10 proteins of the invention. In a specific embodiment, antibodies of the invention bind a heterodimer, a heterotrimer, or a heterotetramer. In additional embodiments, the antibodies of the invention bind at least a homodimer, at least a homotrimer, or at least a homotetramer containing one or more TR10 polypeptides.

Multimers bound by one or more antibodies of the invention may be the result of hydrophobic, hydrophilic, ionic and/or covalent associations and/or may be indirectly linked, by for example, liposome formation. Thus, in one embodiment, multimers bound by one or more antibodies of the invention, such as, for example, homodimers or homotrimers, are formed when TR10 proteins contact one another in solution. In another embodiment, heteromultimers bound by one or more antibodies of the invention, such as, for example, heterotrimers or heterotetramers, are formed when proteins of the invention contact antibodies to the TR10 polypeptides (including antibodies to the heterologous polypeptide sequence in a fusion protein) in solution. In other embodiments, multimers bound by one or more antibodies of the invention are formed by covalent associations with and/or between the TR10 proteins. Such covalent associations may involve one or more amino acid residues contained in the polypeptide sequence of the protein (e.g., the polypeptide sequence recited in SEQ ID NO:4 or the polypeptide encoded by the deposited cDNA clone of ATCC Deposit 209040). In one instance, the covalent associations are cross-linking between cysteine residues located within the polypeptide sequences of the proteins which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a TR10 fusion protein. In one example, covalent associations are between the heterologous sequence contained in a fusion protein (see, e.g., U.S. Pat. No. 5,478,925). In a specific example, the covalent associations are between the heterologous sequence contained in a TR10-Fc fusion protein (as described herein). In another specific example, covalent associations of fusion proteins are between heterologous polypeptide sequences from another TNF family ligand/receptor member that is capable of forming covalently associated multimers, such as for example, osteoprotegerin (see, e.g., International Publication No. WO 98/49305, the contents of which are herein incorporated by reference in its entirety).

The multimers that may be bound by one or more antibodies of the invention may be generated using chemical techniques known in the art. For example, proteins desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers that may be bound by one or more antibodies of the invention may be generated using techniques known in the art to form one or more inter-molecule cross-link-s between the cysteine residues located within the polypeptide sequence of the proteins desired to be contained in the multimer (see, e.g., U.S. Pat. No. 5,478,925, which is herein incorporated by reference in its entirety). Further, proteins that may be bound by one or more antibodies of the invention may be routinely modified by the addition of cysteine or biotin to the C terminus or N-terminus of the polypeptide sequence of the protein and techniques known in the art may be applied to generate multimers containing one or more of these modified proteins (see, e.g., U.S. Pat. No. 5,478,925, which is herein incorporated by reference in its entirety). Additionally, techniques known in the art may be applied to generate liposomes containing the protein components desired to be contained in the multimer that may be bound by one or more antibodies of the invention (see, e.g., U.S. Pat. No. 5,478,925, which is herein incorporated by reference in its entirety).

Alternatively, multimers that may be bound by one or more antibodies of the invention may be generated using genetic engineering techniques known in the art. In one embodiment, proteins contained in multimers that may be bound by one or more antibodies of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer that may be bound by one or more antibodies of the invention are generated by ligating a polynucleotide sequence encoding a TR10 polypeptide to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant TR10 polypeptides which contain a transmembrane domain and which can be incorporated by membrane reconstitution techniques into liposomes (see, e.g., U.S. Pat. No. 5,478,925, which is herein incorporated by reference in its entirety). In another embodiment, two or more TR10 polypeptides are joined through synthetic linkers (e.g., peptide, carbohydrate or soluble polymer linkers). Examples include those peptide linkers described in U.S. Pat. No. 5,073,627 (hereby incorporated by reference). Proteins comprising multiple TR10 polypeptides separated by peptide linkers may be produced using conventional recombinant DNA technology. In specific embodiments, antibodies of the invention bind proteins comprising multiple TR10 polypeptides separated by peptide linkers.

Another method for preparing multimer TR10 polypeptides involves use of TR10 polypeptides fused to a leucine zipper or isoleucine polypeptide sequence. Leucine zipper domains and isoleucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric TR10 proteins are those described in PCT application WO 94/10308, hereby incorporated by reference. Recombinant fusion proteins comprising a soluble TR10 polypeptide fused to a peptide that dimerizes or trimerizes in solution are expressed in suitable host cells, and the resulting soluble multimeric TR10 is recovered from the culture supernatant using techniques known in the art. In specific embodiments, antibodies of the invention bind TR10-leucine zipper fusion protein monomers and/or TR10-leucine zipper fusion protein multimers.

Certain members of the TNF family of proteins are believed to exist in trimeric form (Beutler and Huffel, Science 264:667, 1994; Banner et al., Cell 73:431, 1993). Thus, trimeric TR10 may offer the advantage of enhanced biological activity. Preferred leucine zipper moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. In specific embodiments, antibodies of the invention bind TR10-leucine zipper fusion protein trimers.

Other peptides derived from naturally occurring trimeric proteins may be employed in preparing trimeric TR10. In specific embodiments, antibodies of the invention bind TR10-fusion protein monomers and/or TR10 fusion protein trimers.

The TR10 polypeptides are preferably provided in an isolated form, and preferably are substantially purified. By “isolated polypeptide” is intended a polypeptide removed from its native environment. Thus, a polypeptide produced and/or contained within a recombinant host cell is considered isolated for purposes of the present invention. Also, intended as an “isolated polypeptide” are polypeptides that have been purified, partially or substantially, from a recombinant host cell. For example, a recombinantly produced version of the TR10 polypeptide is substantially purified by the one-step method described in Smith and Johnson, Gene 67:31-40 (1988).

Antibodies of the present invention may bind TR10 polypeptides comprising or alternatively, consisting of, an amino acid sequence contained in SEQ ID NO:4, encoded by the cDNA contained in ATCC Deposit Number 209040, or encoded by nucleic acids which hybridize (e.g., under stringent hybridization conditions) to the nucleotide sequence contained in ATCC Deposit Number 209040, or the complementary strand thereto. Protein fragments may be “free-standing,” or comprised within a larger polypeptide of which the fragment forms a part or region, most preferably as a single continuous region. Antibodies of the present invention may bind polypeptide fragments, including, for example, fragments that comprise or alternatively, consist of from about amino acid residues: 1 to 55, 56 to 105, 106 to 155, 156 to 212, 213 to 230, 231 to 281, 282 to 282, and/or 283 to 386 of SEQ ID NO:4. Moreover, polypeptide fragments can be at least 10, 20, 30, 40, 50, 60, 70, 80, 90, 100, 110, 120, 130, 140, 150, 175 or 200 amino acids in length. In this context “about” includes the particularly recited ranges, larger or smaller by several (5, 4, 3, 2, or 1) amino acids, at either extreme or at both extremes.

In specific embodiments, polypeptide fragments of the invention comprise, or alternatively consist of, amino acid residues: 1-55, 56 to 212, 213 to 230, 231 to 286, and/or 353 to 363 as depicted in SEQ ID NO:4.

Preferably, antibodies of the present invention bind polypeptide fragments selected from the group: (a) a polypeptide comprising or alternatively, consisting of, the TR10 extracellular domain (predicted to constitute amino acid residues from about 56 to about 212 in SEQ ID NO:4; (b) a polypeptide comprising or alternatively, consisting of, both TR10 cysteine rich domains (both of which may be found in the protein fragment consisting of amino acid residues from about 81 to about 182 in SEQ ID NO:4); (c) a polypeptide comprising or alternatively, consisting of, the TR10 cysteine rich domain consisting of amino acid residues from about 81 to about 135 in SEQ ID NO:4); (d) a polypeptide comprising or alternatively, consisting of, the TR10 cysteine rich domain consisting of amino acid residues from about 136 to about 182 in SEQ ID NO:1); (e) a polypeptide comprising or alternatively, consisting of, the TR10 transmembrane domain (predicted to constitute amino acid residues from about 213 to about 230 in SEQ ID NO:4; (f) a polypeptide comprising or alternatively, consisting of, the TR10 intracellular domain (predicted to constitute amino acid residues from about 231 to about 386 in SEQ ID NO:4); (g) a polypeptide comprising or alternatively, consisting of, the TR10 partial death domain (predicted to constitute amino acid residues from about 353 to about 363 in SEQ ID NO:4); (h) a polypeptide comprising, or alternatively, consisting of, one, two, three, four or more, epitope bearing portions of the TR10 receptor protein (g) any combination of polypeptides (a)-(h).

It is believed that one or both of the extracellular cysteine rich motifs of TR10 is important for interactions between TR10 and its ligands (e.g., TRAIL). Accordingly, in highly preferred embodiments, antibodies of the present invention bind TR10 polypeptide fragments comprising, or alternatively consisting of amino acid residues 81 to 135, and/or 136 to 182 of SEQ ID NO:4. In another highly preferred embodiment, antibodies of the present invention bind TR10 polypeptides comprising, or alternatively consisting of both of the extracellular cysteine rich motifs (amino acid residues 81 to 182 of SEQ ID NO:4.) In another preferred embodiment, antibodies of the present invention bind TR10 polypeptides comprising, or alternatively consisting the extracellular soluble domain of TR10 (amino acid residues 56-212 of SEQ ID NO:4.) In highly preferred embodiments, the antibodies of the invention that bind all or a portion of the extracellular soluble domain of TR10 (e.g., one or both cysteine rich domains) prevent TRAIL ligand from binding to TR10. In other highly preferred embodiments, the antibodies of the invention that bind all or a portion of the extracellular soluble domain of TR10 (e.g., one or both cysteine rich domains) agonize the TR10 receptor.

Antibodies of the invention may also bind fragments comprising, or alternatively, consisting of structural or functional attributes of TR10. Such fragments include amino acid residues that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, surface forming regions, and high antigenic index regions (i.e., containing four or more contiguous amino acids having an antigenic index of greater than or equal to 1.5, as identified using the default parameters of the Jameson-Wolf program) of complete (i.e., full-length) TR10. Certain preferred regions are those set out in Table 6 and include, but are not limited to, regions of the aforementioned types identified by analysis of the amino acid sequence depicted in (SEQ ID NO:4), such preferred regions include; Garnier-Robson predicted alpha-regions, beta-regions, turn-regions, and coil-regions; Chou-Fasman predicted alpha-regions, beta-regions, and turn-regions; Kyte-Doolittle predicted hydrophilic regions; Eisenberg alpha and beta amphipathic regions; Emini surface-forming regions; and Jameson-Wolf high antigenic index regions, as predicted using the default parameters of these computer programs.

The data representing the structural or functional attributes of TR10 set forth in Table 6, as described above, was generated using the various modules and algorithms of the DNA*STAR set on default parameters. Column I represents the results of a Garnier-Robson analysis of alpha helical regions; Column II represents the results of a Chou-Fasman analysis of alpha helical regions; Column III represents the results of a Garnier Robson analysis of beta sheet regions; Column IV represents the results of a Chou-Fasman analysis of beta sheet regions; Column V represents the results of a Garnier Robson analysis of turn regions; Column VI represents the results of a Chou-Fasman analysis of turn regions; Column VII represents the results of a Garnier Robson analysis of coil regions; Column VIII represents a Kyte-Doolittle hydrophilicity plot; Column IX represents a Hopp-Woods hydrophobicity plot; Column X represents the results of an Eisenberg analysis of alpha amphipathic regions; Column XI represents the results of an Eisenberg analysis of beta amphipathic regions; Column XII represents the results of a Karplus-Schultz analysis of flexible regions; Column XIII represents the Jameson-Wolf antigenic index score; and Column XIV represents the Emini surface probability plot.

In a preferred embodiment, the data presented in columns VIII, IX, XIII, and XIV of Table 6 can be used to determine regions of TR10 which exhibit a high degree of potential for antigenicity. Regions of high antigenicity are determined from the data presented in columns VIII, IX, XIII, and/or XIV by choosing values which represent regions of the polypeptide which are likely to be exposed on the surface of the polypeptide in an environment in which antigen recognition may occur in the process of initiation of an immune response.

The above-mentioned preferred regions set out in Table 6 include, but are not limited to, regions of the aforementioned types identified by analysis of the amino acid sequence set out in SEQ ID NO:4. As set out in Table 6, such preferred regions include Garnier-Robson alpha-regions, beta-regions, turn-regions, and coil-regions, Chou-Fasman alpha-regions, beta-regions, and turn-regions, Kyte-Doolittle hydrophilic regions, Eisenberg alpha- and beta-amphipathic regions, Karplus-Schulz flexible regions, Jameson-Wolf regions of high antigenic index and Emini surface-forming regions. Preferably, antibodies of the present invention bind TR10 polypeptides or TR10 polypeptide fragments and variants comprising regions of TR10 that combine several structural features, such as several (e.g., 1, 2, 3, or 4) of the same or different region features set out above and in Table 6.

TABLE 6

| Res | Position | I | II | III | IV | V | VI | VII | VIII | IX | X | XI | XII | XIII | XIV |
|---|---|---|---|---|---|---|---|---|---|---|---|---|---|---|---|
| Met | 1 | . | . | B | . | . | . | . | −0.44 | 0.93 | . | . | . | −0.40 | 0.32 |
| Gly | 2 | . | . | B | . | . | . | . | −0.06 | 0.93 | . | . | . | −0.40 | 0.25 |
| Leu | 3 | . | . | . | . | T | . | . | 0.03 | 0.90 | . | . | . | 0.00 | 0.33 |
| Trp | 4 | . | . | . | . | T | . | . | −0.43 | 0.86 | . | . | . | 0.00 | 0.45 |
| Gly | 5 | . | . | . | . | . | . | C | −0.26 | 0.89 | . | . | F | −0.05 | 0.34 |
| Gln | 6 | . | . | B | . | . | . | . | 0.03 | 0.89 | * | . | F | −0.25 | 0.64 |
| Ser | 7 | . | . | B | . | . | . | . | −0.21 | 0.69 | . | . | F | −0.25 | 0.87 |
| Val | 8 | . | . | B | . | . | . | . | 0.30 | 0.27 | * | . | F | 0.05 | 0.89 |
| Pro | 9 | . | . | B | . | . | . | . | 0.29 | 0.23 | * | . | F | 0.05 | 0.69 |
| Thr | 10 | . | . | . | . | . | T | C | 0.04 | 0.21 | . | * | F | 0.45 | 0.69 |
| Ala | 11 | . | . | B | . | . | T | . | 0.16 | 0.33 | . | * | F | 0.25 | 0.94 |
| Ser | 12 | . | . | B | . | . | T | . | −0.13 | −0.31 | . | * | F | 1.00 | 1.19 |
| Ser | 13 | . | . | B | . | . | T | . | 0.38 | −0.24 | * | . | F | 1.13 | 0.83 |
| Ala | 14 | . | . | B | . | . | . | . | 0.70 | −0.30 | * | . | F | 1.21 | 0.81 |
| Arg | 15 | . | . | B | . | . | T | . | 0.77 | −0.80 | * | . | F | 2.14 | 1.19 |
| Ala | 16 | . | . | B | . | . | T | . | 1.14 | −0.43 | * | . | F | 2.12 | 1.39 |
| Gly | 17 | . | . | . | . | T | T | . | 1.10 | −0.39 | * | . | F | 2.80 | 2.13 |
| Arg | 18 | . | . | B | . | . | T | . | 0.81 | −0.46 | * | . | F | 2.12 | 1.08 |
| Tyr | 19 | . | . | B | . | . | T | . | 1.51 | 0.04 | * | . | F | 1.24 | 1.08 |
| Pro | 20 | . | . | B | . | . | T | . | 1.09 | −0.46 | * | . | F | 1.56 | 2.13 |
| Gly | 21 | . | . | B | . | . | T | . | 1.09 | −0.40 | . | . | F | 1.28 | 1.57 |
| Ala | 22 | . | . | B | . | . | T | . | 1.13 | 0.10 | . | . | F | 0.70 | 1.01 |
| Arg | 23 | . | . | B | . | . | . | . | 0.68 | −0.27 | . | . | F | 1.25 | 0.88 |
| Thr | 24 | . | . | B | . | . | . | . | 0.61 | −0.27 | . | * | F | 1.55 | 0.88 |
| Ala | 25 | . | . | B | . | . | T | . | 0.93 | −0.21 | . | * | F | 2.20 | 1.25 |
| Ser | 26 | . | . | . | . | . | T | C | 1.07 | −0.71 | * | * | F | 3.00 | 1.25 |
| Gly | 27 | . | . | . | . | T | T | . | 1.37 | −0.29 | * | * | F | 2.60 | 1.34 |
| Thr | 28 | . | . | . | . | . | T | C | 0.44 | 0.14 | * | * | F | 1.50 | 1.40 |
| Arg | 29 | . | . | B | . | . | T | . | −0.06 | 0.33 | . | . | F | 0.85 | 0.86 |
| Pro | 30 | . | . | B | . | . | T | . | 0.53 | 0.63 | . | . | F | 0.25 | 0.72 |
| Trp | 31 | . | . | B | . | . | T | . | 0.62 | 0.20 | * | . | . | 0.10 | 0.83 |
| Leu | 32 | . | . | B | . | . | T | . | 1.01 | 0.14 | * | * | . | 0.10 | 0.65 |
| Leu | 33 | . | . | B | . | . | . | . | 0.43 | 0.14 | * | * | . | −0.10 | 0.85 |
| Asp | 34 | . | . | B | . | . | T | . | −0.49 | 0.40 | * | . | F | 0.25 | 0.56 |
| Pro | 35 | A | . | . | . | . | T | . | −0.23 | 0.17 | . | * | F | 0.25 | 0.56 |
| Lys | 36 | A | . | . | . | . | T | . | −0.64 | −0.51 | * | * | F | 1.30 | 1.37 |
| Ile | 37 | A | . | . | . | . | T | . | −0.69 | −0.41 | * | . | . | 0.70 | 0.71 |
| Leu | 38 | A | . | . | B | . | . | . | −0.73 | 0.23 | * | * | . | −0.30 | 0.34 |
| Lys | 39 | . | . | B | B | . | . | . | −1.43 | 0.44 | * | * | . | −0.60 | 0.13 |
| Phe | 40 | . | . | B | B | . | . | . | −2.11 | 1.23 | * | . | . | −0.60 | 0.16 |
| Val | 41 | . | . | B | B | . | . | . | −3.01 | 1.23 | * | . | . | −0.60 | 0.13 |
| Val | 42 | . | . | B | B | . | . | . | −2.71 | 1.19 | * | . | . | −0.60 | 0.05 |
| Phe | 43 | . | . | B | B | . | . | . | −2.76 | 1.69 | * | * | . | −0.60 | 0.06 |
| Ile | 44 | . | . | B | B | . | . | . | −3.61 | 1.54 | * | . | . | −0.60 | 0.06 |
| Val | 45 | . | . | B | B | . | . | . | −3.72 | 1.59 | . | . | . | −0.60 | 0.06 |
| Ala | 46 | . | . | B | B | . | . | . | −3.08 | 1.63 | . | . | . | −0.60 | 0.06 |
| Val | 47 | . | . | B | B | . | . | . | −3.08 | 1.27 | . | * | . | −0.60 | 0.13 |
| Leu | 48 | . | . | B | B | . | . | . | −2.27 | 1.23 | * | * | . | −0.60 | 0.13 |
| Leu | 49 | . | . | B | B | . | . | . | −2.23 | 0.59 | . | * | . | −0.60 | 0.26 |
| Pro | 50 | . | . | B | B | . | . | . | −1.38 | 0.73 | . | * | . | −0.60 | 0.26 |
| Val | 51 | . | . | B | B | . | . | . | −1.09 | 0.09 | . | * | . | −0.30 | 0.53 |
| Arg | 52 | . | . | B | B | . | . | . | −0.82 | −0.21 | . | * | . | 0.30 | 0.86 |
| Val | 53 | . | . | B | B | . | . | . | −0.32 | −0.40 | . | * | . | 0.30 | 0.56 |
| Asp | 54 | . | . | B | B | . | . | . | −0.40 | −0.34 | . | * | F | 0.60 | 1.09 |
| Ser | 55 | . | . | B | B | . | . | . | −0.40 | −0.30 | * | * | F | 0.45 | 0.39 |
| Ala | 56 | . | . | B | B | . | . | . | 0.57 | 0.13 | * | * | F | −0.15 | 0.81 |
| Thr | 57 | . | . | B | B | . | . | . | 0.46 | −0.51 | . | * | F | 0.75 | 0.95 |
| Ile | 58 | . | . | B | B | . | . | . | 1.31 | −0.11 | . | . | F | 0.60 | 1.23 |
| Pro | 59 | . | . | . | . | . | . | C | 1.31 | −0.50 | . | . | F | 1.30 | 2.03 |
| Arg | 60 | . | A | . | . | T | . | . | 0.76 | −1.00 | . | . | F | 1.30 | 2.44 |
| Gln | 61 | . | A | . | . | T | . | . | 1.13 | −0.84 | . | . | F | 1.30 | 2.58 |
| Asp | 62 | . | A | . | . | T | . | . | 1.44 | −1.10 | * | . | F | 1.30 | 2.58 |
| Glu | 63 | . | A | B | . | . | . | . | 2.33 | −1.13 | * | . | F | 0.90 | 2.28 |
| Val | 64 | . | A | B | . | . | . | . | 2.23 | −0.73 | * | . | F | 0.90 | 2.28 |
| Pro | 65 | . | . | B | . | . | . | . | 1.27 | −0.64 | * | . | F | 1.10 | 1.97 |
| Gln | 66 | . | . | . | B | T | . | . | 0.68 | −0.00 | . | . | F | 0.85 | 0.85 |
| Gln | 67 | . | . | B | B | . | . | . | 0.47 | 0.50 | . | . | F | −0.30 | 1.15 |
| Thr | 68 | . | . | B | B | . | . | . | 0.47 | 0.29 | . | . | F | 0.00 | 1.15 |
| Val | 69 | . | . | B | B | . | . | . | 1.32 | 0.26 | . | . | F | 0.22 | 1.15 |
| Ala | 70 | . | . | B | B | . | . | . | 1.53 | 0.26 | . | . | F | 0.44 | 1.15 |
| Pro | 71 | . | . | B | B | . | . | . | 1.64 | 0.26 | . | . | F | 0.66 | 1.38 |
| Gln | 72 | . | . | B | . | . | . | . | 1.76 | −0.23 | * | . | F | 1.68 | 3.64 |
| Gln | 73 | . | . | B | . | . | . | . | 1.77 | −0.87 | * | . | F | 2.20 | 7.06 |
| Gln | 74 | . | . | B | . | . | T | . | 1.81 | −0.99 | * | . | F | 2.18 | 6.12 |
| Arg | 75 | . | . | B | . | . | T | . | 2.44 | −0.73 | * | . | F | 1.96 | 2.91 |
| Arg | 76 | . | . | . | . | T | T | . | 2.66 | −1.13 | * | . | F | 2.14 | 3.37 |
| Ser | 77 | . | . | . | . | . | T | C | 2.66 | −1.53 | * | . | F | 1.72 | 3.37 |
| Leu | 78 | . | A | . | . | . | . | C | 2.66 | −1.93 | * | . | F | 1.10 | 2.98 |
| Lys | 79 | . | A | . | . | . | . | C | 1.99 | −1.93 | * | . | F | 1.10 | 2.63 |
| Glu | 80 | A | A | . | . | . | . | . | 1.67 | −1.36 | * | . | F | 0.90 | 1.05 |
| Glu | 81 | A | A | . | . | . | . | . | 0.97 | −1.31 | * | . | F | 0.90 | 1.97 |
| Glu | 82 | A | A | . | . | . | . | . | 0.92 | −1.50 | . | . | F | 0.75 | 1.00 |
| Cys | 83 | A | . | . | . | . | T | . | 1.43 | −1.07 | . | . | F | 1.15 | 0.57 |
| Pro | 84 | A | . | . | . | . | T | . | 1.36 | −0.69 | . | . | F | 1.15 | 0.44 |
| Ala | 85 | A | . | . | . | . | T | . | 1.47 | −0.19 | . | . | F | 0.85 | 0.35 |
| Gly | 86 | A | . | . | . | . | T | . | 1.17 | −0.19 | . | * | F | 1.00 | 1.27 |
| Ser | 87 | A | . | . | . | . | . | . | 1.17 | −0.37 | . | * | F | 0.80 | 1.10 |
| His | 88 | . | . | . | . | . | . | C | 1.59 | −0.80 | . | * | F | 1.58 | 1.88 |
| Arg | 89 | . | . | B | . | . | . | . | 1.49 | −0.54 | . | * | F | 1.66 | 2.98 |
| Ser | 90 | . | . | B | . | . | . | . | 1.73 | −0.49 | . | * | F | 1.64 | 3.21 |
| Glu | 91 | . | . | . | . | T | . | . | 1.49 | −0.44 | . | * | F | 2.32 | 2.33 |
| Tyr | 92 | . | . | . | . | T | T | . | 1.12 | −0.44 | . | * | F | 2.80 | 1.20 |
| Thr | 93 | . | . | . | . | T | T | . | 1.16 | 0.13 | . | * | F | 1.77 | 0.48 |
| Gly | 94 | . | . | . | . | T | T | . | 0.83 | 0.14 | * | * | . | 1.34 | 0.45 |
| Ala | 95 | . | . | . | . | T | T | . | 0.47 | 0.57 | . | . | . | 1.01 | 0.44 |
| Cys | 96 | . | . | . | . | T | . | . | 0.16 | 0.39 | . | . | . | 1.08 | 0.16 |
| Asn | 97 | . | . | . | . | . | T | C | 0.40 | 0.39 | . | . | . | 1.05 | 0.24 |
| Pro | 98 | . | . | . | . | T | T | . | 0.37 | −0.04 | * | . | F | 2.25 | 0.41 |
| Cys | 99 | . | . | . | . | T | T | . | −0.14 | −0.11 | * | . | F | 2.50 | 0.76 |
| Thr | 100 | . | . | B | . | . | T | . | 0.44 | −0.04 | * | . | F | 1.85 | 0.35 |
| Glu | 101 | . | . | B | . | . | . | . | 0.87 | −0.44 | * | . | F | 1.40 | 0.38 |
| Gly | 102 | . | . | B | . | . | T | . | 0.56 | −0.11 | * | * | F | 1.50 | 1.10 |
| Val | 103 | . | . | B | . | . | T | . | −0.12 | −0.20 | . | . | . | 1.10 | 1.10 |
| Asp | 104 | . | . | B | . | . | T | . | −0.04 | −0.00 | . | . | . | 0.70 | 0.45 |
| Tyr | 105 | . | . | B | . | . | T | . | −0.03 | 0.50 | . | * | . | −0.20 | 0.46 |
| Thr | 106 | . | . | B | . | . | . | . | −0.03 | 0.46 | . | . | . | −0.40 | 0.82 |
| Ile | 107 | . | . | B | . | . | . | . | 0.31 | 0.21 | . | * | . | −0.10 | 0.79 |
| Ala | 108 | . | . | B | . | . | T | . | 0.36 | 0.61 | . | * | . | −0.20 | 0.81 |
| Ser | 109 | . | . | . | . | T | T | . | 0.14 | 0.54 | . | . | F | 0.35 | 0.46 |
| Asn | 110 | . | . | . | . | T | T | . | 0.09 | 0.49 | . | . | F | 0.50 | 1.02 |
| Asn | 111 | . | . | . | . | T | T | . | −0.27 | 0.19 | . | . | F | 0.80 | 1.36 |
| Leu | 112 | . | . | . | . | . | T | C | −0.19 | 0.26 | . | . | F | 0.45 | 0.54 |
| Pro | 113 | . | . | . | . | T | T | . | −0.41 | 0.56 | . | . | F | 0.35 | 0.28 |
| Ser | 114 | . | . | . | . | T | T | . | −0.78 | 0.84 | . | . | . | 0.20 | 0.14 |
| Cys | 115 | . | . | B | . | . | T | . | −1.09 | 1.01 | * | . | . | −0.20 | 0.09 |
| Leu | 116 | . | . | B | B | . | . | . | −1.94 | 0.81 | * | . | . | −0.60 | 0.09 |
| Leu | 117 | . | . | B | B | . | . | . | −1.80 | 1.03 | * | . | . | −0.60 | 0.05 |
| Cys | 118 | . | . | B | B | . | . | . | −1.54 | 1.21 | * | . | . | −0.60 | 0.05 |
| Thr | 119 | . | . | B | B | . | . | . | −1.54 | 0.64 | * | . | . | −0.60 | 0.12 |
| Val | 120 | . | . | B | B | . | . | . | −1.22 | 0.34 | * | . | . | −0.30 | 0.19 |
| Cys | 121 | . | . | B | . | . | T | . | −0.41 | 0.09 | * | . | . | 0.10 | 0.35 |
| Lys | 122 | . | . | B | . | . | T | . | 0.09 | −0.09 | * | . | F | 0.85 | 0.42 |
| Ser | 123 | . | . | . | . | T | T | . | 0.76 | −0.09 | . | . | F | 1.59 | 0.81 |
| Gly | 124 | . | . | . | . | T | T | . | 1.11 | −0.33 | . | . | F | 2.08 | 2.44 |
| Gln | 125 | . | . | . | . | T | . | . | 1.67 | −0.90 | . | . | F | 2.52 | 2.44 |
| Thr | 126 | . | . | . | . | T | . | . | 2.03 | −0.51 | . | . | F | 2.86 | 2.44 |
| Asn | 127 | . | . | . | . | T | T | . | 1.32 | −0.51 | . | . | F | 3.40 | 3.31 |
| Lys | 128 | . | . | . | . | T | T | . | 1.31 | −0.37 | . | . | F | 2.76 | 1.02 |
| Ser | 129 | . | . | . | . | T | T | . | 1.34 | −0.29 | . | . | F | 2.42 | 1.02 |
| Ser | 130 | . | . | . | . | T | T | . | 1.03 | −0.29 | * | . | F | 1.93 | 0.92 |
| Cys | 131 | . | . | B | B | . | . | . | 1.46 | −0.20 | * | . | F | 0.79 | 0.66 |
| Thr | 132 | . | . | B | B | . | . | . | 1.46 | −0.20 | * | . | F | 0.45 | 0.97 |
| Thr | 133 | . | . | B | B | . | . | . | 1.10 | −0.59 | * | * | F | 0.90 | 1.21 |
| Thr | 134 | . | . | . | . | T | T | . | 0.54 | −0.49 | . | . | F | 1.40 | 3.25 |
| Arg | 135 | . | . | . | . | T | T | . | 0.18 | −0.41 | . | . | F | 1.40 | 1.67 |
| Asp | 136 | . | . | . | . | T | T | . | 0.84 | −0.33 | * | . | F | 1.25 | 0.62 |
| Thr | 137 | . | . | B | . | . | T | . | 0.49 | −0.41 | * | . | . | 0.70 | 0.75 |
| Val | 138 | . | . | B | B | . | . | . | 0.80 | −0.33 | * | . | . | 0.61 | 0.20 |
| Cys | 139 | . | . | B | B | . | . | . | 1.16 | −0.33 | * | . | . | 0.92 | 0.21 |
| Gln | 140 | . | . | B | B | . | . | . | 0.70 | −0.33 | * | . | . | 1.23 | 0.29 |
| Cys | 141 | . | . | B | B | . | . | . | 0.40 | −0.39 | * | . | . | 1.54 | 0.39 |
| Glu | 142 | . | . | . | . | T | T | . | 0.01 | −0.64 | * | * | F | 3.10 | 0.98 |
| Lys | 143 | . | . | . | . | T | T | . | 0.87 | −0.43 | * | . | F | 2.49 | 0.49 |
| Gly | 144 | . | . | . | . | T | T | . | 1.53 | −0.43 | . | * | F | 2.67 | 1.58 |
| Ser | 145 | . | . | . | . | T | T | . | 1.58 | −1.00 | . | * | F | 3.00 | 1.52 |
| Phe | 146 | A | . | . | . | . | . | . | 2.24 | −1.00 | . | * | F | 2.43 | 1.52 |
| Gln | 147 | . | . | . | . | T | . | . | 1.94 | −0.60 | . | * | F | 2.86 | 2.47 |
| Asp | 148 | . | . | . | . | T | T | . | 1.69 | −0.64 | . | * | F | 3.40 | 2.47 |
| Lys | 149 | . | . | . | . | T | T | . | 2.03 | −0.60 | . | * | F | 3.06 | 4.42 |
| Asn | 150 | . | . | . | . | . | T | C | 1.73 | −1.39 | . | * | F | 2.52 | 4.42 |
| Ser | 151 | . | . | . | . | . | T | C | 1.77 | −1.17 | * | * | F | 2.18 | 2.62 |
| Pro | 152 | . | . | . | . | T | . | . | 1.88 | −0.60 | * | . | F | 1.69 | 0.70 |
| Glu | 153 | . | . | . | . | T | . | . | 1.57 | −0.60 | * | . | F | 1.35 | 0.85 |
| Met | 154 | . | . | B | B | . | . | . | 0.86 | −0.51 | * | . | . | 0.60 | 0.92 |
| Cys | 155 | . | . | B | B | . | . | . | 0.97 | −0.33 | * | . | . | 0.30 | 0.32 |
| Arg | 156 | . | . | B | B | . | . | . | 0.96 | −0.76 | * | . | . | 0.60 | 0.36 |
| Thr | 157 | . | . | B | B | . | . | . | 0.82 | −0.27 | * | . | . | 0.64 | 0.53 |
| Cys | 158 | . | . | . | . | T | T | . | 0.16 | −0.46 | * | . | F | 1.93 | 0.97 |
| Arg | 159 | . | . | . | . | T | T | . | 0.54 | −0.46 | * | . | F | 2.27 | 0.27 |
| Thr | 160 | . | . | . | . | T | T | . | 1.32 | −0.03 | * | . | F | 2.61 | 0.28 |
| Gly | 161 | . | . | . | . | T | T | . | 0.87 | −0.51 | * | . | F | 3.40 | 1.04 |
| Cys | 162 | . | . | . | . | . | T | C | 0.58 | −0.66 | . | * | F | 2.71 | 0.53 |
| Pro | 163 | . | . | . | . | T | T | . | 0.39 | −0.04 | . | * | F | 2.27 | 0.36 |
| Arg | 164 | . | . | . | . | T | T | . | 0.32 | 0.11 | * | * | F | 1.33 | 0.27 |
| Gly | 165 | . | . | B | . | . | T | . | −0.22 | −0.31 | * | * | . | 1.19 | 1.01 |
| Met | 166 | . | . | B | . | . | . | . | −0.18 | −0.24 | * | * | . | 0.50 | 0.48 |
| Val | 167 | . | . | B | . | . | . | . | 0.49 | −0.29 | * | * | . | 0.50 | 0.33 |
| Lys | 168 | . | . | B | . | . | . | . | 0.03 | 0.11 | * | * | . | −0.10 | 0.54 |
| Val | 169 | . | . | B | . | . | T | . | −0.39 | 0.26 | * | * | . | 0.10 | 0.29 |
| Ser | 170 | . | . | B | . | . | T | . | −0.26 | 0.13 | * | * | F | 0.59 | 0.57 |
| Asn | 171 | . | . | B | . | . | T | . | 0.46 | −0.09 | * | * | F | 1.53 | 0.44 |
| Cys | 172 | . | . | B | . | . | T | . | 1.01 | −0.09 | * | * | F | 2.02 | 1.16 |
| Thr | 173 | . | . | B | . | . | T | . | 0.97 | −0.34 | * | * | F | 2.36 | 1.16 |
| Pro | 174 | . | . | . | . | T | T | . | 0.93 | −0.73 | * | * | F | 3.40 | 1.20 |
| Arg | 175 | . | . | . | . | T | T | . | 1.28 | −0.44 | * | * | F | 2.76 | 1.57 |
| Ser | 176 | . | . | B | . | T | T | . | 0.61 | −1.01 | . | * | F | 2.72 | 2.18 |
| Asp | 177 | . | . | . | . | T | . | . | 1.32 | −0.93 | . | * | F | 2.03 | 0.76 |
| Ile | 178 | . | . | B | . | . | . | . | 1.63 | −1.36 | . | * | F | 1.29 | 0.77 |
| Lys | 179 | . | . | B | . | . | . | . | 1.84 | −0.96 | . | * | F | 0.95 | 0.93 |
| Cys | 180 | . | . | B | . | . | T | . | 1.43 | −1.34 | . | * | F | 1.15 | 0.96 |
| Lys | 181 | . | . | B | . | . | T | . | 1.14 | −0.96 | . | * | F | 1.30 | 1.83 |
| Asn | 182 | A | . | . | . | . | T | . | 0.56 | −1.14 | . | * | F | 1.15 | 0.93 |
| Glu | 183 | A | . | . | . | . | T | . | 1.14 | −0.64 | . | * | F | 1.30 | 1.75 |
| Ser | 184 | A | A | . | . | . | . | . | 0.80 | −0.83 | . | * | F | 1.18 | 1.17 |
| Ala | 185 | A | A | . | . | . | . | . | 1.16 | −0.44 | . | . | F | 1.01 | 0.98 |
| Ala | 186 | A | A | . | . | . | . | . | 0.77 | −0.36 | . | . | F | 1.29 | 0.81 |
| Ser | 187 | A | A | . | . | . | . | . | 0.81 | 0.07 | . | . | F | 0.97 | 0.60 |
| Ser | 188 | . | . | . | . | T | T | . | 0.50 | −0.31 | * | . | F | 2.80 | 1.19 |
| Thr | 189 | . | . | . | . | T | T | . | 0.59 | −0.33 | * | * | F | 2.52 | 1.70 |
| Gly | 190 | . | . | . | . | T | T | . | 0.59 | −0.40 | . | . | F | 2.24 | 1.96 |
| Lys | 191 | . | . | . | . | . | T | C | 0.59 | −0.29 | . | . | F | 1.76 | 1.48 |
| Thr | 192 | . | A | . | . | . | . | C | 0.89 | −0.17 | . | . | F | 1.08 | 1.03 |
| Pro | 193 | . | A | . | . | . | . | C | 1.19 | −0.66 | . | . | F | 1.10 | 1.81 |
| Ala | 194 | A | A | . | . | . | . | . | 1.19 | −1.09 | . | . | F | 0.90 | 1.57 |
| Ala | 195 | A | A | . | . | . | . | . | 0.68 | −0.60 | . | . | F | 0.90 | 1.57 |
| Glu | 196 | A | A | . | . | . | . | . | 0.32 | −0.44 | . | . | F | 0.45 | 0.75 |
| Glu | 197 | A | A | . | B | . | . | . | 0.32 | −0.39 | * | . | F | 0.60 | 1.07 |
| Thr | 198 | A | A | . | B | . | . | . | −0.36 | −0.40 | * | . | F | 0.60 | 1.53 |
| Val | 199 | A | . | . | B | . | . | . | −0.58 | −0.21 | * | . | F | 0.45 | 0.62 |
| Thr | 200 | A | . | . | B | . | . | . | −0.33 | 0.47 | * | . | F | −0.45 | 0.30 |
| Thr | 201 | A | . | . | B | . | . | . | −0.93 | 0.90 | * | . | . | −0.60 | 0.20 |
| Ile | 202 | A | A | . | B | . | . | . | −1.74 | 1.03 | * | . | . | −0.60 | 0.27 |
| Leu | 203 | . | A | B | B | . | . | . | −2.02 | 1.07 | * | . | . | −0.60 | 0.15 |
| Gly | 204 | . | A | B | . | . | . | . | −1.47 | 1.09 | * | . | . | −0.60 | 0.11 |
| Met | 205 | . | A | B | . | . | . | . | −1.37 | 0.99 | . | . | . | −0.60 | 0.21 |
| Leu | 206 | . | A | B | . | . | . | . | −1.30 | 0.73 | * | . | . | −0.60 | 0.39 |
| Ala | 207 | . | A | B | . | . | . | . | −0.44 | 0.80 | . | . | . | −0.60 | 0.61 |
| Ser | 208 | . | . | . | . | . | T | C | 0.12 | 0.87 | . | . | . | 0.00 | 0.84 |
| Pro | 209 | A | . | . | . | . | T | . | −0.34 | 1.01 | . | . | . | −0.05 | 1.60 |
| Tyr | 210 | A | . | . | . | . | T | . | −0.63 | 1.01 | . | . | . | −0.05 | 1.31 |
| His | 211 | . | . | B | . | . | T | . | −0.71 | 1.20 | . | . | . | −0.20 | 0.68 |
| Tyr | 212 | . | . | B | B | . | . | . | −1.01 | 1.50 | . | . | . | −0.60 | 0.31 |
| Leu | 213 | . | . | B | B | . | . | . | −1.57 | 1.76 | . | . | . | −0.60 | 0.14 |
| Ile | 214 | . | . | B | B | . | . | . | −2.21 | 1.64 | . | . | . | −0.60 | 0.08 |
| Ile | 215 | . | . | B | B | . | . | . | −2.78 | 1.79 | . | . | . | −0.60 | 0.04 |
| Ile | 216 | . | . | B | B | . | . | . | −3.60 | 1.71 | . | . | . | −0.60 | 0.04 |
| Val | 217 | . | . | B | B | . | . | . | −4.24 | 1.67 | . | . | . | −0.60 | 0.04 |
| Val | 218 | . | . | B | B | . | . | . | −4.32 | 1.67 | . | . | . | −0.60 | 0.04 |
| Leu | 219 | . | . | B | B | . | . | . | −4.24 | 1.67 | . | . | . | −0.60 | 0.04 |
| Val | 220 | . | . | B | B | . | . | . | −3.94 | 1.67 | . | . | . | −0.60 | 0.04 |
| Ile | 221 | . | . | B | B | . | . | . | −3.91 | 1.53 | . | . | . | −0.60 | 0.06 |
| Ile | 222 | . | . | B | B | . | . | . | −3.91 | 1.53 | . | . | . | −0.60 | 0.05 |
| Leu | 223 | . | . | B | B | . | . | . | −3.91 | 1.49 | . | . | . | −0.60 | 0.05 |
| Ala | 224 | . | . | B | B | . | . | . | −3.96 | 1.49 | . | . | . | −0.60 | 0.05 |
| Val | 225 | . | . | B | B | . | . | . | −3.44 | 1.44 | . | . | . | −0.60 | 0.06 |
| Val | 226 | . | . | B | B | . | . | . | −3.26 | 1.19 | . | . | . | −0.60 | 0.07 |
| Val | 227 | . | . | B | B | . | . | . | −2.67 | 1.29 | . | . | . | −0.60 | 0.06 |
| Val | 228 | . | . | B | B | . | . | . | −2.52 | 1.17 | . | * | . | −0.60 | 0.11 |
| Gly | 229 | . | . | B | B | . | . | . | −1.82 | 1.10 | . | * | . | −0.60 | 0.08 |
| Phe | 230 | A | . | . | . | . | T | . | −0.92 | 0.46 | . | * | . | −0.20 | 0.21 |
| Ser | 231 | A | . | . | . | . | T | . | −0.02 | −0.19 | . | * | . | 0.70 | 0.55 |
| Cys | 232 | A | . | . | . | . | T | . | 0.13 | −0.83 | . | * | . | 1.15 | 1.12 |
| Arg | 233 | A | . | . | . | . | T | . | 0.10 | −0.47 | . | * | F | 1.00 | 1.12 |
| Lys | 234 | . | A | . | B | T | . | . | 0.14 | −0.57 | . | * | F | 1.15 | 0.58 |
| Lys | 235 | A | A | . | B | . | . | . | 0.60 | −0.57 | * | * | F | 0.90 | 1.46 |
| Phe | 236 | . | A | B | B | . | . | . | 0.09 | −0.39 | * | * | . | 0.45 | 1.17 |
| Ile | 237 | . | A | B | B | . | . | . | 0.80 | 0.30 | * | * | . | −0.30 | 0.48 |
| Ser | 238 | . | . | B | B | . | . | . | 0.34 | 0.30 | * | * | . | −0.30 | 0.48 |
| Tyr | 239 | . | . | B | B | . | . | . | −0.59 | 0.73 | * | . | . | −0.60 | 0.55 |
| Leu | 240 | . | . | B | B | . | . | . | −1.30 | 0.63 | * | * | . | −0.60 | 0.55 |
| Lys | 241 | . | . | B | B | . | . | . | −0.90 | 0.51 | * | . | . | −0.60 | 0.22 |
| Gly | 242 | . | . | B | B | . | . | . | −0.36 | 0.51 | * | . | . | −0.60 | 0.19 |
| Ile | 243 | . | . | B | B | . | . | . | −0.40 | 0.19 | * | . | . | −0.30 | 0.23 |
| Cys | 244 | . | . | B | . | . | T | . | −0.50 | −0.07 | * | . | . | 0.70 | 0.11 |
| Ser | 245 | . | . | B | . | . | T | . | −0.03 | 0.36 | * | . | F | 0.25 | 0.11 |
| Gly | 246 | . | . | . | . | T | T | . | −0.42 | 0.36 | * | . | F | 0.65 | 0.16 |
| Gly | 247 | . | . | . | . | T | T | . | −0.29 | 0.10 | . | . | F | 0.92 | 0.29 |
| Gly | 248 | . | . | . | . | . | . | C | 0.60 | −0.04 | * | . | F | 1.39 | 0.34 |
| Gly | 249 | . | . | . | . | . | . | C | 1.38 | −0.43 | * | * | F | 1.66 | 0.59 |
| Gly | 250 | . | . | . | . | . | T | C | 0.82 | −0.86 | * | * | F | 2.58 | 1.17 |
| Pro | 251 | . | . | . | . | . | T | C | 1.13 | −0.64 | * | . | F | 2.70 | 0.87 |
| Glu | 252 | . | . | B | . | . | T | . | 1.59 | −0.57 | * | . | F | 2.38 | 1.20 |
| Arg | 253 | . | . | B | . | . | T | . | 1.08 | −1.00 | * | . | F | 2.11 | 2.38 |
| Val | 254 | . | . | B | B | . | . | . | 0.61 | −0.79 | * | . | . | 1.29 | 1.14 |
| His | 255 | . | . | B | B | . | . | . | 0.26 | −0.53 | * | * | . | 0.87 | 0.54 |
| Arg | 256 | . | . | B | B | . | . | . | 0.58 | 0.26 | * | . | . | −0.30 | 0.24 |
| Val | 257 | . | . | B | B | . | . | . | 0.69 | 0.26 | * | * | . | −0.30 | 0.63 |
| Leu | 258 | . | . | B | B | . | . | . | 0.69 | −0.39 | * | * | . | 0.64 | 0.91 |
| Phe | 259 | . | . | B | B | . | . | . | 1.24 | −0.89 | * | * | . | 1.28 | 0.91 |
| Arg | 260 | . | . | B | . | . | T | . | 0.61 | −0.50 | . | * | . | 1.87 | 1.65 |
| Arg | 261 | . | . | . | . | T | T | . | 0.29 | −0.57 | . | * | F | 3.06 | 1.07 |
| Arg | 262 | . | . | . | . | T | T | . | 0.84 | −0.83 | . | * | F | 3.40 | 1.91 |
| Ser | 263 | . | . | . | . | T | T | . | 1.77 | −1.23 | * | * | F | 3.06 | 1.31 |
| Cys | 264 | . | . | . | . | . | T | C | 1.61 | −1.23 | . | * | F | 2.52 | 1.31 |
| Pro | 265 | . | . | . | . | T | T | . | 1.29 | −0.59 | . | * | F | 2.23 | 0.50 |
| Ser | 266 | . | . | . | . | T | T | . | 0.83 | −0.16 | . | * | F | 1.86 | 0.57 |
| Arg | 267 | . | . | B | . | . | T | . | 0.13 | −0.11 | * | . | F | 1.54 | 1.06 |
| Val | 268 | . | . | B | . | . | T | . | 0.43 | −0.19 | . | * | F | 1.66 | 0.69 |
| Pro | 269 | . | . | B | . | . | T | . | 1.10 | −0.61 | . | * | F | 2.23 | 0.89 |
| Gly | 270 | . | . | . | . | . | T | C | 1.31 | −1.00 | * | * | F | 2.70 | 0.76 |
| Ala | 271 | A | . | B | . | . | T | . | 1.02 | −0.60 | . | * | F | 2.38 | 1.65 |
| Glu | 272 | A | . | . | . | . | . | . | 1.02 | −0.74 | * | * | F | 1.91 | 1.08 |
| Asp | 273 | A | . | . | . | . | . | . | 1.88 | −1.17 | * | * | F | 1.64 | 2.13 |
| Asn | 274 | A | . | . | . | . | T | . | 2.09 | −1.20 | * | * | F | 1.57 | 3.39 |
| Ala | 275 | A | . | . | . | . | T | . | 2.12 | −1.70 | . | * | F | 1.30 | 3.39 |
| Arg | 276 | A | . | . | . | . | T | . | 1.90 | −1.21 | . | * | F | 1.64 | 2.93 |
| Asn | 277 | A | . | . | . | . | T | . | 1.60 | −0.53 | . | * | F | 1.98 | 1.50 |
| Glu | 278 | A | . | . | . | . | . | . | 1.60 | −0.54 | . | * | F | 2.12 | 1.99 |
| Thr | 279 | A | . | . | . | . | . | . | 1.71 | −0.64 | . | * | F | 2.46 | 1.64 |
| Leu | 280 | . | . | . | . | T | T | . | 2.06 | −0.64 | * | . | F | 3.40 | 1.99 |
| Ser | 281 | . | . | . | . | T | T | . | 1.13 | −0.29 | * | . | F | 2.76 | 1.80 |
| Asn | 282 | . | . | . | . | T | T | . | 1.13 | 0.40 | * | . | F | 1.52 | 1.03 |
| Arg | 283 | . | . | . | . | T | T | . | 0.92 | 0.31 | * | . | F | 1.48 | 2.17 |
| Tyr | 284 | . | . | . | . | T | . | . | 0.92 | 0.06 | * | . | F | 0.94 | 2.50 |
| Leu | 285 | . | . | B | . | . | . | . | 1.73 | 0.16 | * | . | F | 0.20 | 2.24 |
| Gln | 286 | . | . | B | . | . | T | . | 1.18 | 0.16 | . | . | F | 0.40 | 1.98 |
| Pro | 287 | . | . | . | . | . | T | C | 0.88 | 0.80 | . | . | F | 0.15 | 0.94 |
| Thr | 288 | . | . | . | . | . | T | C | 0.77 | 0.43 | . | . | F | 0.30 | 1.53 |
| Gln | 289 | . | . | B | . | . | T | . | 1.01 | −0.26 | . | . | F | 1.00 | 1.53 |
| Val | 290 | . | A | B | . | . | . | . | 1.82 | −0.26 | . | . | F | 0.60 | 1.71 |
| Ser | 291 | . | A | B | . | . | . | . | 0.93 | −0.69 | * | . | F | 0.90 | 2.05 |
| Glu | 292 | . | A | B | . | . | . | . | 1.14 | −0.49 | * | * | F | 0.45 | 0.83 |
| Gln | 293 | . | A | B | . | . | . | . | 1.11 | −0.49 | . | * | F | 0.60 | 1.94 |
| Glu | 294 | A | A | . | . | . | . | . | 1.11 | −0.70 | . | * | F | 0.90 | 1.43 |
| Ile | 295 | A | A | . | . | . | . | . | 1.97 | −0.69 | . | . | F | 0.90 | 1.43 |
| Gln | 296 | A | A | . | . | . | . | . | 1.46 | −0.69 | . | * | F | 0.90 | 1.43 |
| Gly | 297 | A | A | . | . | . | . | . | 0.87 | −0.40 | . | * | F | 0.45 | 0.68 |
| Gln | 298 | A | A | . | . | . | . | . | 0.87 | 0.10 | . | * | F | −0.15 | 0.98 |
| Glu | 299 | A | A | . | . | . | . | . | 0.06 | −0.59 | . | * | F | 0.75 | 0.98 |
| Leu | 300 | A | A | . | . | . | . | . | 0.63 | −0.30 | . | * | F | 0.45 | 0.82 |
| Ala | 301 | A | A | . | . | . | . | . | 0.29 | −0.24 | . | . | . | 0.30 | 0.68 |
| Glu | 302 | A | A | . | . | . | . | . | −0.22 | −0.21 | . | . | . | 0.30 | 0.39 |
| Leu | 303 | A | A | . | B | . | . | . | −0.53 | 0.43 | . | . | . | −0.60 | 0.35 |
| Thr | 304 | A | A | . | B | . | . | . | −1.39 | 0.23 | . | . | . | −0.30 | 0.50 |
| Gly | 305 | A | A | . | B | . | . | . | −0.58 | 0.37 | . | . | . | −0.30 | 0.21 |
| Val | 306 | . | . | B | B | . | . | . | −0.29 | 0.37 | . | . | . | −0.30 | 0.45 |
| Thr | 307 | . | . | B | B | . | . | . | −0.50 | 0.07 | . | . | F | 0.15 | 0.42 |
| Val | 308 | . | . | . | B | . | . | C | 0.31 | 0.01 | . | . | F | 0.65 | 0.65 |
| Glu | 309 | . | . | . | B | . | . | C | 0.62 | −0.41 | . | . | F | 1.70 | 1.53 |
| Ser | 310 | . | . | . | . | . | T | C | 0.76 | −1.06 | . | * | F | 2.70 | 1.83 |
| Pro | 311 | . | . | . | . | . | T | C | 1.61 | −1.11 | * | * | F | 3.00 | 3.82 |
| Glu | 312 | A | . | . | . | . | T | . | 2.03 | −1.36 | * | * | F | 2.50 | 3.82 |
| Glu | 313 | A | . | . | . | . | T | . | 2.08 | −1.36 | * | . | F | 2.20 | 5.58 |
| Pro | 314 | A | . | . | . | . | . | . | 1.27 | −1.06 | * | . | F | 1.70 | 2.97 |
| Gln | 315 | A | A | . | . | . | . | . | 1.57 | −0.80 | * | . | F | 1.20 | 1.42 |
| Arg | 316 | A | A | . | . | . | . | . | 1.78 | −0.80 | * | . | F | 0.90 | 1.42 |
| Leu | 317 | A | A | . | . | . | . | . | 1.19 | −0.40 | * | * | F | 0.60 | 1.59 |
| Leu | 318 | A | A | . | . | . | . | . | 1.19 | −0.33 | * | * | F | 0.45 | 0.93 |
| Glu | 319 | A | A | . | . | . | . | . | 0.81 | −0.73 | * | * | F | 0.75 | 0.82 |
| Gln | 320 | A | A | . | . | . | . | . | 0.81 | −0.23 | * | * | F | 0.60 | 1.00 |
| Ala | 321 | A | A | . | . | . | . | . | 0.36 | −0.91 | * | * | F | 0.90 | 2.10 |
| Glu | 322 | A | A | . | . | . | . | . | 0.50 | −1.17 | * | . | F | 0.90 | 1.20 |
| Ala | 323 | A | A | . | . | . | . | . | 1.31 | −0.60 | * | * | F | 0.75 | 0.37 |
| Glu | 324 | A | A | . | . | . | . | . | 1.42 | −0.60 | . | * | F | 0.75 | 0.64 |
| Gly | 325 | A | A | . | . | . | . | . | 1.53 | −1.10 | . | * | F | 0.75 | 0.72 |
| Cys | 326 | A | A | . | . | . | . | . | 2.23 | −1.10 | . | * | F | 0.90 | 1.40 |
| Gln | 327 | A | A | . | . | . | . | . | 1.42 | −1.60 | . | * | F | 0.90 | 1.58 |
| Arg | 328 | A | A | . | . | . | . | . | 1.20 | −0.91 | . | * | F | 0.90 | 1.32 |
| Arg | 329 | . | A | B | B | . | . | . | 0.34 | −0.66 | . | * | F | 0.90 | 2.03 |
| Arg | 330 | . | A | B | B | . | . | . | 0.48 | −0.59 | . | * | . | 0.60 | 0.87 |
| Leu | 331 | . | A | B | B | . | . | . | 0.29 | −0.56 | . | * | . | 0.60 | 0.69 |
| Leu | 332 | . | A | B | B | . | . | . | 0.29 | 0.09 | * | * | . | −0.30 | 0.26 |
| Val | 333 | . | A | B | B | . | . | . | 0.18 | 0.49 | * | * | . | −0.60 | 0.21 |
| Pro | 334 | . | A | B | . | . | . | . | −0.52 | 0.49 | * | . | . | −0.60 | 0.43 |
| Val | 335 | . | . | B | . | . | . | . | −0.63 | 0.30 | * | * | . | −0.10 | 0.53 |
| Asn | 336 | . | . | B | . | . | . | . | −0.12 | −0.39 | . | . | F | 0.80 | 1.19 |
| Asp | 337 | A | . | . | . | . | T | . | 0.10 | −0.64 | . | . | F | 1.30 | 1.03 |
| Ala | 338 | A | . | . | . | . | T | . | 0.96 | −0.57 | . | . | F | 1.30 | 1.41 |
| Asp | 339 | A | . | . | . | . | T | . | 0.28 | −1.21 | . | . | F | 1.30 | 1.46 |
| Ser | 340 | A | . | . | . | . | T | . | 0.83 | −0.93 | . | . | F | 1.15 | 0.61 |
| Ala | 341 | A | . | . | . | . | . | . | 0.52 | −0.54 | . | . | F | 0.95 | 0.81 |
| Asp | 342 | A | . | . | B | . | . | . | −0.29 | −0.56 | . | . | F | 0.75 | 0.70 |
| Ile | 343 | A | . | . | B | . | . | . | −0.51 | 0.13 | . | * | F | −0.15 | 0.43 |
| Ser | 344 | . | A | B | B | . | . | . | −0.51 | 0.43 | . | * | F | −0.45 | 0.35 |
| Thr | 345 | . | A | B | B | . | . | . | −0.80 | −0.07 | . | * | F | 0.45 | 0.35 |
| Leu | 346 | A | A | . | B | . | . | . | −0.51 | 0.43 | * | * | . | −0.60 | 0.51 |
| Leu | 347 | A | A | . | B | . | . | . | −1.10 | 0.13 | * | * | . | −0.30 | 0.51 |
| Asp | 348 | A | A | . | B | . | . | . | −0.52 | 0.24 | * | * | . | −0.30 | 0.36 |
| Ala | 349 | A | A | . | . | . | . | . | −1.03 | 0.24 | . | * | . | −0.30 | 0.62 |
| Ser | 350 | A | A | . | . | . | . | . | −0.72 | 0.24 | * | * | . | −0.30 | 0.62 |
| Ala | 351 | A | A | . | . | . | . | . | 0.09 | −0.44 | * | * | . | 0.30 | 0.65 |
| Thr | 352 | A | A | . | . | . | . | . | 0.56 | −0.44 | . | * | . | 0.45 | 1.11 |
| Leu | 353 | A | A | . | . | . | . | . | 0.52 | −0.51 | . | * | F | 0.75 | 0.82 |
| Glu | 354 | A | A | . | . | . | . | . | 0.52 | −0.40 | . | * | F | 0.60 | 1.10 |
| Glu | 355 | A | A | . | . | . | . | . | 0.87 | −0.40 | . | . | F | 0.45 | 0.77 |
| Gly | 356 | A | A | . | . | . | . | . | 1.46 | −0.89 | . | . | F | 0.90 | 1.87 |
| His | 357 | A | A | . | . | . | . | . | 1.46 | −1.57 | . | * | F | 0.90 | 1.87 |
| Ala | 358 | A | A | . | . | . | . | . | 1.38 | −1.09 | . | * | F | 0.90 | 1.56 |
| Lys | 359 | A | A | . | . | . | . | . | 1.38 | −0.40 | . | * | F | 0.60 | 1.10 |
| Glu | 360 | A | A | . | . | . | . | . | 1.38 | −0.43 | . | * | F | 0.60 | 1.41 |
| Thr | 361 | A | A | . | . | . | . | . | 1.72 | −0.93 | * | * | F | 0.90 | 2.32 |
| Ile | 362 | A | A | . | . | . | . | . | 0.94 | −1.03 | . | * | F | 0.90 | 2.01 |
| Gln | 363 | A | A | . | . | . | . | . | 0.68 | −0.34 | . | * | F | 0.45 | 0.96 |
| Asp | 364 | A | A | . | . | . | . | . | 0.29 | 0.30 | . | * | F | −0.15 | 0.49 |
| Gln | 365 | A | A | . | . | . | . | . | −0.01 | 0.24 | . | . | F | 0.06 | 0.70 |
| Leu | 366 | . | A | B | . | . | . | . | 0.30 | −0.06 | * | * | F | 0.87 | 0.54 |
| Val | 367 | . | A | B | . | . | . | . | 1.23 | −0.46 | * | * | F | 1.08 | 0.56 |
| Gly | 368 | . | . | . | . | . | T | C | 0.42 | −0.46 | * | . | F | 1.89 | 0.64 |
| Ser | 369 | . | . | . | . | . | T | C | −0.28 | −0.17 | * | . | F | 2.10 | 0.64 |
| Glu | 370 | A | . | . | . | . | T | . | −0.52 | −0.07 | . | * | F | 1.69 | 0.75 |
| Lys | 371 | A | . | . | . | . | T | . | 0.29 | 0.04 | . | . | F | 1.03 | 1.19 |
| Leu | 372 | A | A | . | . | . | . | . | 1.14 | −0.39 | . | . | F | 1.02 | 1.54 |
| Phe | 373 | A | A | . | . | . | . | . | 1.49 | −0.77 | * | . | . | 0.96 | 1.54 |
| Tyr | 374 | A | A | . | . | . | . | . | 1.79 | −0.77 | * | . | . | 0.75 | 1.28 |
| Glu | 375 | A | A | . | . | . | . | . | 1.20 | −0.77 | * | . | F | 0.90 | 2.70 |
| Glu | 376 | A | A | . | . | . | . | . | 0.81 | −0.96 | * | . | F | 0.90 | 3.15 |
| Asp | 377 | A | A | . | . | . | . | . | 1.32 | −1.31 | . | . | F | 0.90 | 1.99 |
| Glu | 378 | A | . | . | . | . | T | . | 1.43 | −1.69 | . | . | F | 1.30 | 1.54 |
| Ala | 379 | A | . | . | . | . | T | . | 1.37 | −1.19 | . | . | F | 1.15 | 0.90 |
| Gly | 380 | A | . | . | . | . | T | . | 1.07 | −0.70 | . | . | F | 1.15 | 0.78 |
| Ser | 381 | A | . | . | . | . | T | . | 0.40 | −0.31 | * | . | F | 0.85 | 0.60 |
| Ala | 382 | A | . | . | . | . | T | . | −0.41 | 0.26 | * | . | F | 0.25 | 0.32 |
| Thr | 383 | A | . | . | . | . | T | . | −0.80 | 0.44 | . | . | F | −0.05 | 0.27 |
| Ser | 384 | A | . | . | . | . | T | . | −0.60 | 0.44 | . | . | F | −0.05 | 0.25 |
| Cys | 385 | . | . | B | . | . | T | . | −0.64 | 0.49 | . | . | . | −0.20 | 0.32 |
| Leu | 386 | . | . | B | . | . | . | . | −0.73 | 0.41 | . | . | . | −0.40 | 0.28 |

In another aspect, the invention provides an antibody that binds a peptide or polypeptide comprising an epitope-bearing portion of a polypeptide described herein. The epitope of this polypeptide portion is an immunogenic or antigenic epitope of a polypeptide of the invention. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe, J. G., Shinnick, T. M., Green, N. and Learner, R. A. (1983) Antibodies that react with predetermined sites on proteins. Science 219:660-666. Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides are therefore useful to raise antibodies, including monoclonal antibodies, that bind to a TR10 polypeptide of the invention. See, for instance, Wilson et al., Cell 37:767-778 (1984) at 777. Antigenic epitope-bearing peptides and polypeptides preferably contain a sequence of at least seven, more preferably at least nine and most preferably between at least about 15 to about 30 amino acids contained within the amino acid sequence of SEQ ID NO:4.

Antibodies of the invention may bind one or more antigenic TR10 polypeptides or peptides including, but not limited to: a polypeptide comprising amino acid residues from about 57 to about 113 of SEQ ID NO:4; a polypeptide comprising amino acid residues from about 130 to about 197 of SEQ ID NO:4; a polypeptide comprising amino acid residues from about 75 to about 142 of SEQ ID NO:4; a polypeptide comprising amino acid residues from about 280 to about 283 of SEQ ID NO:4; and/or a polypeptide comprising amino acid residues from about 195 to about 228 of SEQ ID NO:4. In this context “about” includes the particularly recited range, larger or smaller by several (5, 4, 3, 2, or 1) amino acids, at either terminus or at both termini. As indicated above, the inventors have determined that the above polypeptide fragments are antigenic regions of the TR10 protein.

Epitope-bearing TR10 peptides and polypeptides may be produced by any conventional means. Houghten, R. A., “General method for the rapid solid-phase synthesis of large numbers of peptides: specificity of antigen-antibody interaction at the level of individual amino acids,” Proc. Natl. Acad. Sci. USA 82:5131-5135 (1985). This “Simultaneous Multiple Peptide Synthesis (SMPS)” process is further described in U.S. Pat. No. 4,631,211 to Houghten et al. (1986).

As one of skill in the art will appreciate, TR10 polypeptides and the epitope-bearing fragments thereof described herein (e.g., corresponding to a portion of the extracellular domain such as, for example, amino acid residues 56 to 212 of SEQ ID NO:4 can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric TR10 protein or protein fragment alone (Fountoulakis et al., J Biochem 270:3958-3964 (1995)). Thus, antibodies of the invention may bind fusion proteins that comprise all or a portion of a TRAIL receptor polypeptide such as TR10.

Recombinant DNA technology known to those skilled in the art can be used to create novel mutant proteins or “muteins” including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions. Antibodies of the present invention may also bind such modified TR10 polypeptides or TR10 polypeptide fragments or variants.

For instance, for many proteins, including the extracellular domain of a membrane associated protein or the mature form(s) of a secreted protein, it is known in the art that one or more amino acids may be deleted from the N-terminus or C-terminus without substantial loss of biological function, or loss of the ability to be bound by a specific antibody. For instance, Ron et al., J. Biol. Chem., 268:2984-2988 (1993) reported modified KGF proteins that had heparin binding activity even if 3, 8, or 27 amino-terminal amino acid residues were missing.

As mentioned above, even if deletion of one or more amino acids from the N-terminus of a protein results in modification of loss of one or more biological functions of the protein, other functional activities (e.g., biological activities, ability to multimerize, ability to bind TR10 ligand) may still be retained. For example, the ability of shortened TR10 polypeptides to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a TR10 polypeptide with a large number of deleted N-terminal amino acid residues may retain some biological or immunogenic activities. In fact, peptides composed of as few as six TR10 amino acid residues may often evoke an immune response.

Accordingly, the present invention further provides antibodies that bind polypeptides having one or more residues deleted from the amino terminus of the TR10 amino acid sequence SEQ ID NO:4 up to the alanine residue at position number 382 and polynucleotides encoding such polypeptides. In particular, the present invention provides polypeptides comprising the amino acid sequence of residues n⁷-386 of SEQ ID NO:4, where n⁷ is an integer from 2 to 381 corresponding to the position of the amino acid residue in SEQ ID NO:4.

More in particular, the invention provides antibodies that bind polypeptides comprising, or alternatively consisting of, the amino acid sequence of residues of G-2 to L-386; L-3 to L-386; W-4 to L-386; G-5 to L-386; Q-6 to L-386; S-7 to L-386; V-8 to L-386; P-9 to L-386; T-10 to L-386; A-11 to L-386; S-12 to L-386; S-13 to L-386; A-14 to L-386; R-15 to L-386; A-16 to L-386; G-17 to L-386; R-18 to L-386; Y-19 to L-386; P-20 to L-386; G-21 to L-386; A-22 to L-386; R-23 to L-386; T-24 to L-386; A-25 to L-386; S-26 to L-386; G-27 to L-386; T-28 to L-386; R-29 to L-386; P-30 to L-386; W-31 to L-386; L-32 to L-386; L-33 to L-386; D-34 to L-386; P-35 to L-386; K-36 to L-386; I-37 to L-386; L-38 to L-386; K-39 to L-386; F-40 to L-386; V-41 to L-386; V-42 to L-386; F-43 to L-386; I-44 to L-386; V-45 to L-386; A-46 to L-386; V-47 to L-386; L-48 to L-386; L-49 to L-386; P-50 to L-386; V-51 to L-386; R-52 to L-386; V-53 to L-386; D-54 to L-386; S-55 to L-386; A-56 to L-386; T-57 to L-386; I-58 to L-386; P-59 to L-386; R-60 to L-386; Q-61 to L-386; D-62 to L-386; E-63 to L-386; V-64 to L-386; P-65 to L-386; Q-66 to L-386; Q-67 to L-386; T-68 to L-386; V-69 to L-386; A-70 to L-386; P-71 to L-386; Q-72 to L-386; Q-73 to L-386; Q-74 to L-386; R-75 to L-386; R-76 to L-386; S-77 to L-386; L-78 to L-386; K-79 to L-386; E-80 to L-386; E-81 to L-386; E-82 to L-386; C-83 to L-386; P-84 to L-386; A-85 to L-386; G-86 to L-386; S-87 to L-386; H-88 to L-386; R-89 to L-386; S-90 to L-386; E-91 to L-386; Y-92 to L-386; T-93 to L-386; G-94 to L-386; A-95 to L-386; C-96 to L-386; N-97 to L-386; P-98 to L-386; C-99 to L-386; T-100 to L-386; E-101 to L-386; G-102 to L-386; V-103 to L-386; D-104 to L-386; Y-105 to L-386; T-106 to L-386; I-107 to L-386; A-108 to L-386; S-109 to L-386; N-110 to L-386; N-111 to L-386; L-112 to L-386; P-113 to L-386; S-114 to L-386; C-115 to L-386; L-116 to L-386; L-117 to L-386; C-118 to L-386; T-119 to L-386; V-120 to L-386; C-121 to L-386; K-122 to L-386; S-123 to L-386; G-124 to L-386; Q-125 to L-386; T-126 to L-386; N-127 to L-386; K-128 to L-386; S-129 to L-386; S-130 to L-386; C-131 to L-386; T-132 to L-386; T-133 to L-386; T-134 to L-386; R-135 to L-386; D-136 to L-386; T-137 to L-386; V-138 to L-386; C-139 to L-386; Q-140 to L-386; C-141 to L-386; E-142 to L-386; K-143 to L-386; G-144 to L-386; S-145 to L-386; F-146 to L-386; Q-147 to L-386; D-148 to L-386; K-149 to L-386; N-150 to L-386; S-151 to L-386; P-152 to L-386; E-153 to L-386; M-154 to L-386; C-155 to L-386; R-156 to L-386; T-157 to L-386; C-158 to L-386; R-159 to L-386; T-160 to L-386; G-161 to L-386; C-162 to L-386; P-163 to L-386; R-164 to L-386; G-165 to L-386; M-166 to L-386; V-167 to L-386; K-168 to L-386; V-169 to L-386; S-170 to L-386; N-171 to L-386; C-172 to L-386; T-173 to L-386; P-174 to L-386; R-175 to L-386; S-176 to L-386; D-177 to L-386; I-178 to L-386; K-179 to L-386; C-180 to L-386; K-181 to L-386; N-182 to L-386; E-183 to L-386; S-184 to L-386; A-185 to L-386; A-186 to L-386; S-187 to L-386; S-188 to L-386; T-189 to L-386; G-190 to L-386; K-191 to L-386; T-192 to L-386; P-193 to L-386; A-194 to L-386; A-195 to L-386; E-196 to L-386; E-197 to L-386; T-198 to L-386; V-199 to L-386; T-200 to L-386; T-201 to L-386; I-202 to L-386; L-203 to L-386; G-204 to L-386; M-205 to L-386; L-206 to L-386; A-207 to L-386; S-208 to L-386; P-209 to L-386; Y-210 to L-386; H-211 to L-386; Y-212 to L-386; L-213 to L-386; I-214 to L-386; I-215 to L-386; I-216 to L-386; V-217 to L-386; V-218 to L-386; L-219 to L-386; V-220 to L-386; I-221 to L-386; I-222 to L-386; L-223 to L-386; A-224 to L-386; V-225 to L-386; V-226 to L-386; V-227 to L-386; V-228 to L-386; G-229 to L-386; F-230 to L-386; S-231 to L-386; C-232 to L-386; R-233 to L-386; K-234 to L-386; K-235 to L-386; F-236 to L-386; I-237 to L-386; S-238 to L-386; Y-239 to L-386; L-240 to L-386; K-241 to L-386; G-242 to L-386; I-243 to L-386; C-244 to L-386; S-245 to L-386; G-246 to L-386; G-247 to L-386; G-248 to L-386; G-249 to L-386; G-250 to L-386; P-251 to L-386; E-252 to L-386; R-253 to L-386; V-254 to L-386; H-255 to L-386; R-256 to L-386; V-257 to L-386; L-258 to L-386; F-259 to L-386; R-260 to L-386; R-261 to L-386; R-262 to L-386; S-263 to L-386; C-264 to L-386; P-265 to L-386; S-266 to L-386; R-267 to L-386; V-268 to L-386; P-269 to L-386; G-270 to L-386; A-271 to L-386; E-272 to L-386; D-273 to L-386; N-274 to L-386; A-275 to L-386; R-276 to L-386; N-277 to L-386; E-278 to L-386; T-279 to L-386; L-280 to L-386; S-281 to L-386; N-282 to L-386; R-283 to L-386; Y-284 to L-386; L-285 to L-386; Q-286 to L-386; P-287 to L-386; T-288 to L-386; Q-289 to L-386; V-290 to L-386; S-291 to L-386; E-292 to L-386; Q-293 to L-386; E-294 to L-386; I-295 to L-386; Q-296 to L-386; G-297 to L-386; Q-298 to L-386; E-299 to L-386; L-300 to L-386; A-301 to L-386; E-302 to L-386; L-303 to L-386; T-304 to L-386; G-305 to L-386; V-306 to L-386; T-307 to L-386; V-308 to L-386; E-309 to L-386; S-310 to L-386; P-311 to L-386; E-312 to L-386; E-313 to L-386; P-314 to L-386; Q-315 to L-386; R-316 to L-386; L-317 to L-386; L-318 to L-386; E-319 to L-386; Q-320 to L-386; A-321 to L-386; E-322 to L-386; A-323 to L-386; E-324 to L-386; G-325 to L-386; C-326 to L-386; Q-327 to L-386; R-328 to L-386; R-329 to L-386; R-330 to L-386; L-331 to L-386; L-332 to L-386; V-333 to L-386; P-334 to L-386; V-335 to L-386; N-336 to L-386; D-337 to L-386; A-338 to L-386; D-339 to L-386; S-340 to L-386; A-341 to L-386; D-342 to L-386; I-343 to L-386; S-344 to L-386; T-345 to L-386; L-346 to L-386; L-347 to L-386; D-348 to L-386; A-349 to L-386; S-350 to L-386; A-351 to L-386; T-352 to L-386; L-353 to L-386; E-354 to L-386; E-355 to L-386; G-356 to L-386; H-357 to L-386; A-358 to L-386; K-359 to L-386; E-360 to L-386; T-361 to L-386; I-362 to L-386; Q-363 to L-386; D-364 to L-386; Q-365 to L-386; L-366 to L-386; V-367 to L-386; G-368 to L-386; S-369 to L-386; E-370 to L-386; K-371 to L-386; L-372 to L-386; F-373 to L-386; Y-374 to L-386; E-375 to L-386; E-376 to L-386; D-377 to L-386; E-378 to L-386; A-379 to L-386; G-380 to L-386; and/or S-381 to L-386 of the TR10 sequence shown in SEQ ID NO:4.

In another embodiment; N-terminal deletions of the TR10 polypeptide can be described by the general formula n⁸-212, where n⁸ is a number from 2 to 207, corresponding to the position of amino acid identified in SEQ ID NO:4.

In specific embodiments, antibodies of the invention bind N terminal deletions of the TR10 comprising, or alternatively consisting of, the amino acid sequence of residues: G-2 to Y-212; L-3 to Y-212; W-4 to Y-212; G-5 to Y-212; Q-6 to Y-212; S-7 to Y-212; V-8 to Y-212; P-9 to Y-212; T-10 to Y-212; A-11 to Y-212; S-12 to Y-212; S-13 to Y-212; A-14 to Y-212; R-15 to Y-212; A-16 to Y-212; G-17 to Y-212; R-18 to Y-212; Y-19 to Y-212; P-20 to Y-212; G-21 to Y-212; A-22 to Y-212; R-23 to Y-212; T-24 to Y-212; A-25 to Y-212; S-26 to Y-212; G-27 to Y-212; T-28 to Y-212; R-29 to Y-212; P-30 to Y-212; W-31 to Y-212; L-32 to Y-212; L-33 to Y-212; D-34 to Y-212; P-35 to Y-212; K-36 to Y-212; I-37 to Y-212; L-38 to Y-212; K-39 to Y-212; F-40 to Y-212; V-41 to Y-212; V-42 to Y-212; F-43 to Y-212; I-44 to Y-212; V-45 to Y-212; A-46 to Y-212; V-47 to Y-212; L-48 to Y-212; L-49 to Y-212; P-50 to Y-212; V-51 to Y-212; R-52 to Y-212; V-53 to Y-212; D-54 to Y-212; S-55 to Y-212; A-56 to Y-212; T-57 to Y-212; I-58 to Y-212; P-59 to Y-212; R-60 to Y-212; Q-61 to Y-212; D-62 to Y-212; E-63 to Y-212; V-64 to Y-212; P-65 to Y-212; Q-66 to Y-212; Q-67 to Y-212; T-68 to Y-212; V-69 to Y-212; A-70 to Y-212; P-71 to Y-212; Q-72 to Y-212; Q-73 to Y-212; Q-74 to Y-212; R-75 to Y-212; R-76 to Y-212; S-77 to Y-212; L-78 to Y-212; K-79 to Y-212; E-80 to Y-212; E-81 to Y-212; E-82 to Y-212; C-83 to Y-212; P-84 to Y-212; A-85 to Y-212; G-86 to Y-212; S-87 to Y-212; H-88 to Y-212; R-89 to Y-212; S-90 to Y-212; E-91 to Y-212; Y-92 to Y-212; T-93 to Y-212; G-94 to Y-212; A-95 to Y-212; C-96 to Y-212; N-97 to Y-212; P-98 to Y-212; C-99 to Y-212; T-100 to Y-212; E-101 to Y-212; G-102 to Y-212; V-103 to Y-212; D-104 to Y-212; Y-105 to Y-212; T-106 to Y-212; I-107 to Y-212; A-108 to Y-212; S-109 to Y-212; N-110 to Y-212; N-111 to Y-212; L-112 to Y-212; P-113 to Y-212; S-114 to Y-212; C-115 to Y-212; L-116 to Y-212; L-117 to Y-212; C-118 to Y-212; T-119 to Y-212; V-120 to Y-212; C-121 to Y-212; K-122 to Y-212; S-123 to Y-212; G-124 to Y-212; Q-125 to Y-212; T-126 to Y-212; N-127 to Y-212; K-128 to Y-212; S-129 to Y-212; S-130 to Y-212; C-131 to Y-212; T-132 to Y-212; T-133 to Y-212; T-134 to Y-212; R-135 to Y-212; D-136 to Y-212; T-137 to Y-212; V-138 to Y-212; C-139 to Y-212; Q-140 to Y-212; C-141 to Y-212; E-142 to Y-212; K-143 to Y-212; G-144 to Y-212; S-145 to Y-212; F-146 to Y-212; Q-147 to Y-212; D-148 to Y-212; K-149 to Y-212; N-150 to Y-212; S-151 to Y-212; P-152 to Y-212; E-153 to Y-212; M-154 to Y-212; C-155 to Y-212; R-156 to Y-212; T-157 to Y-212; C-158 to Y-212; R-159 to Y-212; T-160 to Y-212; G-161 to Y-212; C-162 to Y-212; P-163 to Y-212; R-164 to Y-212; G-165 to Y-212; M-166 to Y-212; V-167 to Y-212; K-168 to Y-212; V-169 to Y-212; S-170 to Y-212; N-171 to Y-212; C-172 to Y-212; T-173 to Y-212; P-174 to Y-212; R-175 to Y-212; S-176 to Y-212; D-177 to Y-212; I-178 to Y-212; K-179 to Y-212; C-180 to Y-212; K-181 to Y-212; N-182 to Y-212; E-183 to Y-212; S-184 to Y-212; A-185 to Y-212; A-186 to Y-212; S-187 to Y-212; S-188 to Y-212; T-189 to Y-212; G-190 to Y-212; K-191 to Y-212; T-192 to Y-212; P-193 to Y-212; A-194 to Y-212; A-195 to Y-212; E-196 to Y-212; E-197 to Y-212; T-198 to Y-212; V-199 to Y-212; T-200 to Y-212; T-201 to Y-212; I-202 to Y-212; L-203 to Y-212; G-204 to Y-212; M-205 to Y-212; L-206 to Y-212; and/or A-207 to Y-212 of the TR10 extracellular domain sequence shown in SEQ ID NO:4.

Also as mentioned above, even if deletion of one or more amino acids from the C-terminus of a protein results in modification of loss of one or more biological functions of the protein, other functional activities (e.g., biological activities (e.g., ability to inhibit TRAIL induced cell death in vivo or in vitro, and/or regulate (e.g., inhibit) B cell proliferation, and/or regulate hematopoiesis), ability to multimerize, ability to bind TR10 ligand (e.g., TRAIL, and/or ligands on the surface of NK cells and/or endothelial cells) may still be retained. For example the ability of the shortened TR10 polypeptide to induce and/or bind to antibodies which recognize the complete or mature forms of the polypeptide generally will be retained when less than the majority of the residues of the complete or mature polypeptide are removed from the C-terminus. Whether a particular polypeptide lacking C-terminal residues of a complete polypeptide retains such immunologic activities can readily be determined by routine methods described herein and otherwise known in the art. It is not unlikely that an TR10 polypeptide with a large number of deleted C-terminal amino acid residues may retain some biological or immunogenic activities. In fact, peptides composed of as few as six TR10 amino acid residues may often evoke an immune response.

Accordingly, the present invention further provides antibodies that bind polypeptides having one or more residues deleted from the carboxy terminus of the amino acid sequence of the TR10 polypeptide shown in SEQ ID NO:4, up to the glutamine residue at position number 61, and polynucleotides encoding such polypeptides. In particular, the present invention provides polypeptides comprising the amino acid sequence of residues 56-m⁷ of SEQ ID NO:4, where m⁷ is an integer from 61 to 385 corresponding to the position of the amino acid residue in SEQ ID NO:4.

More in particular, the invention provides antibodies that bind polypeptides comprising, or alternatively consisting of, the amino acid sequence of residues A-56 to C-385; A-56 to S-384; A-56 to T-383; A-56 to A-382; A-56 to S-381; A-56 to G-380; A-56 to A-379; A-56 to E-378; A-56 to D-377; A-56 to E-376; A-56 to E-375; A-56 to Y-374; A-56 to F-373; A-56 to L-372; A-56 to K-371; A-56 to E-370; A-56 to S-369; A-56 to G-368; A-56 to V-367; A-56 to L-366; A-56 to Q-365; A-56 to D-364; A-56 to Q-363; A-56 to I-362; A-56 to T-361; A-56 to E-360; A-56 to K-359; A-56 to A-358; A-56 to H-357; A-56 to G-356; A-56 to E-355; A-56 to E-354; A-56 to L-353; A-56 to T-352; A-56 to A-351; A-56 to S-350; A-56 to A-349; A-56 to D-348; A-56 to L-347; A-56 to L-346; A-56 to T-345; A-56 to S-344; A-56 to I-343; A-56 to D-342; A-56 to A-341; A-56 to S-340; A-56 to D-339; A-56 to A-338; A-56 to D-337; A-56 to N-336; A-56 to V-335; A-56 to P-334; A-56 to V-333; A-56 to L-332; A-56 to L-331; A-56 to R-330; A-56 to R-329; A-56 to R-328; A-56 to Q-327; A-56 to C-326; A-56 to G-325; A-56 to E-324; A-56 to A-323; A-56 to E-322; A-56 to A-321; A-56 to Q-320; A-56 to E-319; A-56 to L-318; A-56 to L-317; A-56 to R-316; A-56 to Q-315; A-56 to P-314; A-56 to E-313; A-56 to E-312; A-56 to P-311; A-56 to S-310; A-56 to E-309; A-56 to V-308; A-56 to T-307; A-56 to V-306; A-56 to G-305; A-56 to T-304; A-56 to L-303; A-56 to E-302; A-56 to A-301; A-56 to L-300; A-56 to E-299; A-56 to Q-298; A-56 to G-297; A-56 to Q-296; A-56 to I-295; A-56 to E-294; A-56 to Q-293; A-56 to E-292; A-56 to S-291; A-56 to V-290; A-56 to Q-289; A-56 to T-288; A-56 to P-287; A-56 to Q-286; A-56 to L-285; A-56 to Y-284; A-56 to R-283; A-56 to N-282; A-56 to S-281; A-56 to L-280; A-56 to T-279; A-56 to E-278; A-56 to N-277; A-56 to R-276; A-56 to A-275; A-56 to N-274; A-56 to D-273; A-56 to E-272; A-56 to A-271; A-56 to G-270; A-56 to P-269; A-56 to V-268; A-56 to R-267; A-56 to S-266; A-56 to P-265; A-56 to C-264; A-56 to S-263; A-56 to R-262; A-56 to R-261; A-56 to R-260; A-56 to F-259; A-56 to L-258; A-56 to V-257; A-56 to R-256; A-56 to H-255; A-56 to V-254; A-56 to R-253; A-56 to E-252; A-56 to P-251; A-56 to G-250; A-56 to G-249; A-56 to G-248; A-56 to G-247; A-56 to G-246; A-56 to S-245; A-56 to C-244; A-56 to I-243; A-56 to G-242; A-56 to K-241; A-56 to L-240; A-56 to Y-239; A-56 to S-238; A-56 to I-237; A-56 to F-236; A-56 to K-235; A-56 to K-234; A-56 to R-233; A-56 to C-232; A-56 to S-231; A-56 to F-230; A-56 to G-229; A-56 to V-228; A-56 to V-227; A-56 to V-226; A-56 to V-225; A-56 to A-224; A-56 to L-223; A-56 to I-222; A-56 to I-221; A-56 to V-220; A-56 to L-219; A-56 to V-218; A-56 to V-217; A-56 to I-216; A-56 to I-215; A-56 to I-214; A-56 to L-213; A-56 to Y-212; A-56 to H-211; A-56 to Y-210; A-56 to P-209; A-56 to S-208; A-56 to A-207; A-56 to L-206; A-56 to M-205; A-56 to G-204; A-56 to L-203; A-56 to I-202; A-56 to T-201; A-56 to T-200; A-56 to V-199; A-56 to T-198; A-56 to E-197; A-56 to E-196; A-56 to A-195; A-56 to A-194; A-56 to P-193; A-56 to T-192; A-56 to K-191; A-56 to G-190; A-56 to T-189; A-56 to S-188; A-56 to S-187; A-56 to A-186; A-56 to A-185; A-56 to S-184; A-56 to E-183; A-56 to N-182; A-56 to K-181; A-56 to C-180; A-56 to K-179; A-56 to I-178; A-56 to D-177; A-56 to S-176; A-56 to R-175; A-56 to P-174; A-56 to T-173; A-56 to C-172; A-56 to N-171; A-56 to S-170; A-56 to V-169; A-56 to K-168; A-56 to V-167; A-56 to M-166; A-56 to G-165; A-56 to R-164; A-56 to P-163; A-56 to C-162; A-56 to G-161; A-56 to T-160; A-56 to R-159; A-56 to C-158; A-56 to T-157; A-56 to R-156; A-56 to C-155; A-56 to M-154; A-56 to E-153; A-56 to P-152; A-56 to S-151; A-56 to N-150; A-56 to K-149; A-56 to D-148; A-56 to Q-147; A-56 to F-146; A-56 to S-145; A-56 to G-144; A-56 to K-143; A-56 to E-142; A-56 to C-141; A-56 to Q-140; A-56 to C-139; A-56 to V-138; A-56 to T-137; A-56 to D-136; A-56 to R-135; A-56 to T-134; A-56 to T-133; A-56 to T-132; A-56 to C-131; A-56 to S-130; A-56 to S-129; A-56 to K-128; A-56 to N-127; A-56 to T-126; A-56 to Q-125; A-56 to G-124; A-56 to S-123; A-56 to K-122; A-56 to C-121; A-56 to V-120; A-56 to T-119; A-56 to C-118; A-56 to L-117; A-56 to L-116; A-56 to C-115; A-56 to S-114; A-56 to P-113; A-56 to L-112; A-56 to N-111; A-56 to N-110; A-56 to S-109; A-56 to A-108; A-56 to I-107; A-56 to T-106; A-56 to Y-105; A-56 to D-104; A-56 to V-103; A-56 to G-102; A-56 to E-101; A-56 to T-100; A-56 to C-99; A-56 to P-98; A-56 to N-97; A-56 to C-96; A-56 to A-95; A-56 to G-94; A-56 to T-93; A-56 to Y-92; A-56 to E-91; A-56 to S-90; A-56 to R-89; A-56 to H-88; A-56 to S-87; A-56 to G-86; A-56 to A-85; A-56 to P-84; A-56 to C-83; A-56 to E-82; A-56 to E-81; A-56 to E-80; A-56 to K-79; A-56 to L-78; A-56 to S-77; A-56 to R-76; A-56 to R-75; A-56 to Q-74; A-56 to Q-73; A-56 to Q-72; A-56 to P-71; A-56 to A-70; A-56 to V-69; A-56 to T-68; A-56 to Q-67; A-56 to Q-66; A-56 to P-65; A-56 to V-64; A-56 to E-63; A-56 to D-62; and/or A-56 to Q-61 of the TR10 sequence shown in SEQ ID NO:4.

In another embodiment, antibodies of the invention bind polypeptides having one or more residues deleted from the carboxy terminus of the amino acid sequence of the TR10 polypeptide shown in SEQ ID NO:4, up to the glutamine residue at position number 61, and polynucleotides encoding such polypeptides. In particular, the present invention provides polypeptides comprising the amino acid sequence of residues 56-m⁸ of SEQ ID NO:4, where m⁸ is an integer from 61 to 212 corresponding to the position of the amino acid residue in SEQ ID NO:4.

More in particular, the invention provides antibodies that bind polypeptides comprising, or alternatively consisting of, the amino acid sequence of residues A-56 to C-385; A-56 to S-384; A-56 to T-383; A-56 to A-382; A-56 to S-381; A-56 to G-380; A-56 to A-379; A-56 to E-378; A-56 to D-377; A-56 to E-376; A-56 to E-375; A-56 to Y-374; A-56 to F-373; A-56 to L-372; A-56 to K-371; A-56 to E-370; A-56 to S-369; A-56 to G-368; A-56 to V-367; A-56 to L-366; A-56 to Q-365; A-56 to D-364; A-56 to Q-363; A-56 to I-362; A-56 to T-361; A-56 to E-360; A-56 to K-359; A-56 to A-358; A-56 to H-357; A-56 to G-356; A-56 to E-355; A-56 to E-354; A-56 to L-353; A-56 to T-352; A-56 to A-351; A-56 to S-350; A-56 to A-349; A-56 to D-348; A-56 to L-347; A-56 to L-346; A-56 to T-345; A-56 to S-344; A-56 to I-343; A-56 to D-342; A-56 to A-341; A-56 to S-340; A-56 to D-339; A-56 to A-338; A-56 to D-337; A-56 to N-336; A-56 to V-335; A-56 to P-334; A-56 to V-333; A-56 to L-332; A-56 to L-331; A-56 to R-330; A-56 to R-329; A-56 to R-328; A-56 to Q-327; A-56 to C-326; A-56 to G-325; A-56 to E-324; A-56 to A-323; A-56 to E-322; A-56 to A-321; A-56 to Q-320; A-56 to E-319; A-56 to L-318; A-56 to L-317; A-56 to R-316; A-56 to Q-315; A-56 to P-314; A-56 to E-313; A-56 to E-312; A-56 to P-311; A-56 to S-310; A-56 to E-309; A-56 to V-308; A-56 to T-307; A-56 to V-306; A-56 to G-305; A-56 to T-304; A-56 to L-303; A-56 to E-302; A-56 to A-301; A-56 to L-300; A-56 to E-299; A-56 to Q-298; A-56 to G-297; A-56 to Q-296; A-56 to I-295; A-56 to E-294; A-56 to Q-293; A-56 to E-292; A-56 to S-291; A-56 to V-290; A-56 to Q-289; A-56 to T-288; A-56 to P-287; A-56 to Q-286; A-56 to L-285; A-56 to Y-284; A-56 to R-283; A-56 to N-282; A-56 to S-281; A-56 to L-280; A-56 to T-279; A-56 to E-278; A-56 to N-277; A-56 to R-276; A-56 to A-275; A-56 to N-274; A-56 to D-273; A-56 to E-272; A-56 to A-271; A-56 to G-270; A-56 to P-269; A-56 to V-268; A-56 to R-267; A-56 to S-266; A-56 to P-265; A-56 to C-264; A-56 to S-263; A-56 to R-262; A-56 to R-261; A-56 to R-260; A-56 to F-259; A-56 to L-258; A-56 to V-257; A-56 to R-256; A-56 to H-255; A-56 to V-254; A-56 to R-253; A-56 to E-252; A-56 to P-251; A-56 to G-250; A-56 to G-249; A-56 to G-248; A-56 to G-247; A-56 to G-246; A-56 to S-245; A-56 to C-244; A-56 to I-243; A-56 to G-242; A-56 to K-241; A-56 to L-240; A-56 to Y-239; A-56 to S-238; A-56 to I-237; A-56 to F-236; A-56 to K-235; A-56 to K-234; A-56 to R-233; A-56 to C-232; A-56 to S-231; A-56 to F-230; A-56 to G-229; A-56 to V-228; A-56 to V-227; A-56 to V-226; A-56 to V-225; A-56 to A-224; A-56 to L-223; A-56 to I-222; A-56 to I-221; A-56 to V-220; A-56 to L-219; A-56 to V-218; A-56 to V-217; A-56 to I-216; A-56 to I-215; A-56 to I-214; A-56 to L-213; and/or A-56 to Y-212; of the TR10 sequence shown in SEQ ID NO:4.

The invention also provides polypeptides having one or more amino acids deleted from both the amino and the carboxyl termini, which may be described generally as having residues n⁷-m⁷ and/or n⁸-m⁸ of SEQ ID NO:4 (i.e., SEQ ID NO:4), where n⁷, n⁸, m⁷ and m⁸ are integers as described above. Thus, any of the above listed N- or C-terminal deletions can be combined to produce a N- and C-terminal deleted TR10 polypeptide.

Also included are antibodies that bind a polypeptide consisting of a portion of the complete TR10 amino acid sequence encoded by the cDNA clone contained in ATCC Deposit No. 209040, where this portion excludes from 1 to about 80 amino acids from the amino terminus of the complete amino acid sequence encoded by the cDNA clone contained in ATCC Deposit No. 209040, or from 1 to about 204 amino acids from the carboxy terminus, or any combination of the above amino terminal and carboxy terminal deletions, of the complete amino acid sequence encoded by the cDNA clone contained in ATCC Deposit No. 209040.

Preferably, antibodies of the present invention bind fragments of TR10 comprising a portion of the extracellular domain; i.e., within residues 56 to 212 of SEQ ID NO:4, since any portion therein is expected to be soluble.

It will be recognized in the art that some amino acid sequences of TR10 can be varied without significant effect on the structure or function of the protein. If such differences in sequence are contemplated, it should be remembered that there will be critical areas on the protein which determine activity. Thus, the invention further includes antibodies that bind variations of the TR10 receptor, which show substantial TR10 receptor activity or which include regions of TR10 proteins, such as the protein portions discussed herein. Such mutants include deletions, insertions, inversions, repeats, and type substitutions. As indicated above, guidance concerning which amino acid changes are likely to be phenotypically silent can be found in J. U. Bowie et al., Science 247:1306-1310 (1990).

The antibodies of the present invention may bind a fragment, derivative, or analog of the polypeptide of SEQ ID NO:4, or that encoded by the deposited cDNA in ATCC deposit 209040. Such fragments, variants or derivatives may be (i) one in which at least one or more of the amino acid residues are substituted with a conserved or non-conserved amino acid residue (preferably a conserved amino acid residue(s), and more preferably at least one but less than ten conserved amino acid residues) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fe fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Of particular interest are substitutions of charged amino acids with another charged amino acid and with neutral or negatively charged amino acids. The latter results in proteins with reduced positive charge to improve the characteristics of the TR10 receptor protein. The prevention of aggregation is highly desirable. Aggregation of proteins not only results in a loss of activity but can also be problematic when preparing pharmaceutical formulations, because they can be immunogenic. (Pinckard et al., Clin Exp. Immunol 2:331-340 (1967); Robbins et al., Diabetes 36:838-845 (1987); Cleland et al. Crit. Rev. Therapeutic Drug Carrier Systems 10:307-377 (1993)).

The replacement of amino acids can also change the selectivity of binding to cell surface receptors. Ostade et al., Nature 361:266-268 (1993), describes certain mutations resulting in selective binding of TNF-α to only one of the two known types of TNF receptors. Thus, the antibodies of the present invention may bind a TR10 receptor that contains one or more amino acid substitutions, deletions, or additions, either from natural mutations or human manipulation.

As indicated, changes are preferably of a minor nature, such as conservative amino acid substitutions that do not significantly affect the folding or activity of the protein (see Table 3 above). In specific embodiments, the number of substitutions, additions or deletions in the amino acid sequence of SEQ ID NO:4 and/or any of the polypeptide fragments described herein (e.g., the extracellular domain or intracellular domain) is 75, 70, 60, 50, 40, 35, 30, 25, 20, 15, 10, 9, 8, 7, 6, 5, 4, 3, 2, 1 or 30-20, 20-15, 20-10, 15-10, 10-1, 5-10, 1-5, 1-3 or 1-2.

In specific embodiments, the antibodies of the invention bind TR10 polypeptides or fragments or variants thereof (especially a fragment comprising or alternatively consisting of, the extracellular soluble domain of TR10), that contains any one or more of the following conservative mutations in TR10: M1 replaced with A, G, I, L, S, T, or V; G2 replaced with A, I, L, S, T, M, or V; L3 replaced with A, G, I, S, T, M, or V; W4 replaced with F, or Y; G5 replaced with A, I, L, S, T, M, or V; Q6 replaced with N; S7 replaced with A, G, I, L, T, M, or V; V8 replaced with A, G, I, L, S, T, or M; T10 replaced with A, G, I, L, S, M, or V; A11 replaced with G, I, L, S, T, M, or V; S12 replaced with A, G, I, L, T, M, or V; S13 replaced with A, G, I, L, T, M, or V; A14 replaced with G, I, L, S, T, M, or V; R15 replaced with H, or K; A16 replaced with G, I, L, S, T, M, or V; G17 replaced with A, I, L, S, T, M, or V; R18 replaced with H, or K; Y19 replaced with F, or W; G21 replaced with A, I, L, S, T, M, or V; A22 replaced with G, I, L, S, T, M, or V; R23 replaced with H, or K; T24 replaced with A, G, I, L, S, M, or V; A25 replaced with G, I, L, S, T, M, or V; S26 replaced with A, G, I, L, T, M, or V; G27 replaced with A, I, L, S, T, M, or V; T28 replaced with A, G, L, L, S, M, or V; R29 replaced with H, or K; W31 replaced with F, or Y; L32 replaced with A, G, I, S, T, M, or V; L33 replaced with A, G, I, S, T, M, or V; D34 replaced with E; K36 replaced with H, or R; I37 replaced with A, G, L, S, T, M, or V; L38 replaced with A, G, I, S, T, M, or V; K39 replaced with H, or R; F40 replaced with W, or Y; V41 replaced with A, G, I, L, S, T, or M; V42 replaced with A, G, I, L, S, T, or M; F43 replaced with W, or Y; I44 replaced with A, G, L, S, T, M, or V; V45 replaced with A, G, I, L, S, T, or M; A46 replaced with G, I, L, S, T, M, or V; V47 replaced with A, G, I, L, S, T, or M; L48 replaced with A, G, I, S, T, M, or V; L49 replaced with A, G, I, S, T, M, or V; V51 replaced with A, G, I, L, S, T, or M; R52 replaced with H, or K; V53 replaced with A, G, I, L, S, T, or M; D54 replaced with E; S55 replaced with A, G, I, L, T, M, or V; A56 replaced with G, I, L, S, T, M, or V; T57 replaced with A, G, I, L, S, M, or V; I58 replaced with A, G, L, S, T, M, or V; R60 replaced with H, or K; Q61 replaced with N; D62 replaced with E; E63 replaced with D; V64 replaced with A, G, I, L, S, T, or M; Q66 replaced with N; Q67 replaced with N; T68 replaced with A, G, I, L, S, M, or V; V69 replaced with A, G, I, L, S, T, or M; A70 replaced with G, I, L, S, T, M, or V; Q72 replaced with N; Q73 replaced with N; Q74 replaced with N; R75 replaced with H, or K; R76 replaced with H, or K; S77 replaced with A, G, I, L, T, M, or V; L78 replaced with A, G, I, S, T, M, or V; K79 replaced with H, or R; E80 replaced with D; E81 replaced with D; E82 replaced with D; A85 replaced with G, I, L, S, T, M, or V; G86 replaced with A, I, L, S, T, M, or V; S87 replaced with A, G, I, L, T, M, or V; H88 replaced with K, or R; R89 replaced with H, or K; S90 replaced with A, G, I, L, T, M, or V; E91 replaced with D; Y92 replaced with F, or W; T93 replaced with A, G, I, L, S, M, or V; G94 replaced with A, I, L, S, T, M, or V; A95 replaced with G, I, L, S, T, M, or V; N97 replaced with Q; T100 replaced with A, G, I, L, S, M, or V; E101 replaced with D; G102 replaced with A, I, L, S, T, M, or V; V103 replaced with A, G, I, L, S, T, or M; D104 replaced with E; Y105 replaced with F, or W; T106 replaced with A, G, I, L, S, M, or V; I107 replaced with A, G, L, S, T, M, or V; A108 replaced with G, I, L, S, T, M, or V; S109 replaced with A, G, I, L, T, M, or V; N110 replaced with Q; N111 replaced with Q; L112 replaced with A, G, I, S, T, M, or V; S114 replaced with A, G, I, L, T, M, or V; L116 replaced with A, G, I, S, T, M, or V; L117 replaced with A, G, I, S, T, M, or V; T119 replaced with A, G, I, L, S, M, or V; V120 replaced with A, G, I, L, S, T, or M; K122 replaced with H, or R; S123 replaced with A, G, I, L, T, M, or V; G124 replaced with A, I, L, S, T, M, or V; Q125 replaced with N; T126 replaced with A, G, I, L, S, M, or V; N127 replaced with Q; K128 replaced with H, or R; S129 replaced with A, G, I, L, T, M, or V; S130 replaced with A, G, I, L, T, M, or V; T132 replaced with A, G, I, L, S, M, or V; T133 replaced with A, G, I, L, S, M, or V; T134 replaced with A, G, I, L, S, M, or V; R135 replaced with H, or K; D136 replaced with E; T137 replaced with A, G, I, L, S, M, or V; V138 replaced with A, G, I, L, S, T, or M; Q140 replaced with N; E142 replaced with D; K143 replaced with H, or R; G144 replaced with A, I, L, S, T, M, or V; S145 replaced with A, G, I, L, T, M, or V; F146 replaced with W, or Y; Q147 replaced with N; D148 replaced with E; K149 replaced with H, or R; N150 replaced with Q; S151 replaced with A, G, I, L, T, M, or V; E153 replaced with D; M154 replaced with A, G, I, L, S, T, or V; R156 replaced with H, or K; T157 replaced with A, G, I, L, S, M, or V; R159 replaced with H, or K; T160 replaced with A, G, I, L, S, M, or V; G161 replaced with A, I, L, S, T, M, or V; R164 replaced with H, or K; G165 replaced with A, I, L, S, T, M, or V; M166 replaced with A, G, I, L, S, T, or V; V167 replaced with A, G, I, L, S, T, or M; K168 replaced with H, or R; V169 replaced with A, G, I, L, S, T, or M; S170 replaced with A, G, I, L, T, M, or V; N171 replaced with Q; T173 replaced with A, G, I, L, S, M, or V; R175 replaced with H, or K; S176 replaced with A, G, I, L, T, M, or V; D177 replaced with E; I178 replaced with A, G, L, S, T, M, or V; K179 replaced with H, or R; K181 replaced with H, or R; N182 replaced with Q; E183 replaced with D; S184 replaced with A, G, I, L, T, M, or V; A185 replaced with G, I, L, S, T, M, or V; A186 replaced with G, I, L, S, T, M, or V; S187 replaced with A, G, I, L, T, M, or V; S188 replaced with A, G, I, L, T, M, or V; T189 replaced with A, G, I, L, S, M, or V; G190 replaced with A, I, L, S, T, M, or V; K191 replaced with H, or R; T192 replaced with A, G, I, L, S, M, or V; A194 replaced with G, I, L, S, T, M, or V; A195 replaced with G, L, S, T, M, or V; E196 replaced with D; E197 replaced with D; T198 replaced with A, G, I, L, S, M, or V; V199 replaced with A, G, I, L, S, T, or M; T200 replaced with A, G, I, L, S, M, or V; T201 replaced with A, G, I, L, S, M, or V; I202 replaced with A, G, L, S, T, M, or V; L203 replaced with A, G, I, S, T, M, or V; G204 replaced with A, I, L, S, T, M, or V; M205 replaced with A, G, I, L, S, T, or V; L206 replaced with A, G, I, S, T, M, or V; A207 replaced with G, I, L, S, T, M, or V; S208 replaced with A, G, I, L, T, M, or V; Y210 replaced with F, or W; H211 replaced with K, or R; Y212 replaced with F, or W; L213 replaced with A, G, I, S, T, M, or V; I214 replaced with A, G, I, L, S, T, M, or V; I215 replaced with A, G, L, S, T, M, or V; I216 replaced with A, G, L, S, T, M, or V; V217 replaced with A, G, I, L, S, T, or M; V218 replaced with A, G, I, L, S, T, or M; L219 replaced with A, G, I, S, T, M, or V; V220 replaced with A, G, I, L, S, T, or M; I221 replaced with A, G, L, S, T, M, or V; I222 replaced with A, G, L, S, T, M, or V; L223 replaced with A, G, I, S, T, M, or V; A224 replaced with G, I, L, S, T, M, or V; V225 replaced with A, G, I, L, S, T, or M; V226 replaced with A, G, I, L, S, T, or M; V227 replaced with A, G, I, L, S, T, or M; V228 replaced with A, G, I, L, S, T, or M; G229 replaced with A, I, L, S, T, M, or V; F230 replaced with W, or Y; S231 replaced with A, G, I, L, T, M, or V; R233 replaced with H, or K; K234 replaced with H, or R; K235 replaced with H, or R; F236 replaced with W, or Y; I237 replaced with A, G, L, S, T, M, or V; S238 replaced with A, G, I, L, T, M, or V; Y239 replaced with F, or W; L240 replaced with A, G, I, S, T, M, or V; K241 replaced with H, or R; G242 replaced with A, I, L, S, T, M, or V; I243 replaced with A, G, L, S, T, M, or V; S245 replaced with A, G, I, L, T, M, or V; G246 replaced with A, L, L, S, T, M, or V; G247 replaced with A, I, L, S, T, M, or V; G248 replaced with A, I, L, S, T, M, or V; G249 replaced with A, L, L, S, T, M, or V; G250 replaced with A, I, L, S, T, M, or V; E252 replaced with D; R253 replaced with H, or K; V254 replaced with A, G, I, L, S, T, or M; H255 replaced with K, or R; R256 replaced with H, or K; V257 replaced with A, G, I, L, S, T, or M; L258 replaced with A, G, I, S, T, M, or V; F259 replaced with W, or Y; R260 replaced with H, or K; R261 replaced with H, or K; R262 replaced with H, or K; S263 replaced with A, G, I, L, T, M, or V; S266 replaced with A, G, I, L, T, M, or V; R267 replaced with H, or K; V268 replaced with A, G, I, L, S, T, or M; G270 replaced with A, I, L, S, T, M, or V; A271 replaced with G, I, L, S, T, M, or V; E272 replaced with D; D273 replaced with E; N274 replaced with Q; A275 replaced with G, I, L, S, T, M, or V; R276 replaced with H, or K; N277 replaced with Q; E278 replaced with D; T279 replaced with A, G, I, L, S, M, or V; L280 replaced with A, G, I, S, T, M, or V; S281 replaced with A, G, I, L, T, M, or V; N282 replaced with Q; R283 replaced with H, or K; Y284 replaced with F, or W; L285 replaced with A, G, I, S, T, M, or V; Q286 replaced with N; T288 replaced with A, G, I, L, S, M, or V; Q289 replaced with N; V290 replaced with A, G, I, L, S, T, or M; S291 replaced with A, G, I, L, T, M, or V; E292 replaced with D; Q293 replaced with N; E294 replaced with D; I295 replaced with A, G, I, L, S, T, M, or V; Q296 replaced with N; G297 replaced with A, I, L, S, T, M, or V; Q298 replaced with N; E299 replaced with D; L300 replaced with A, G, I, S, T, M, or V; A301 replaced with G, I, L, S, T, M, or V; E302 replaced with D; L303 replaced with A, G, I, S, T, M, or V; T304 replaced with A, G, I, L, S, M, or V; G305 replaced with A, I, L, S, T, M, or V; V306 replaced with A, G, I, L, S, T, or M; T307 replaced with A, G, I, L, S, M, or V; V308 replaced with A, G, I, L, S, T, or M; E309 replaced with D; S310 replaced with A, G, I, L, T, M, or V; E312 replaced with D; E313 replaced with D; Q315 replaced with N; R316 replaced with H, or K; L317 replaced with A, G, I, S, T, M, or V; L318 replaced with A, G, I, S, T, M, or V; E-319 replaced with D; Q320 replaced with N; A321 replaced with G, I, L, S, T, M, or V; E322 replaced with D; A323 replaced with G, I, L, S, T, M, or V; E324 replaced with D; G325 replaced with A, I, L, S, T, M, or V; Q327 replaced with N; R328 replaced with H, or K; R329 replaced with H, or K; R330 replaced with H, or K; L331 replaced with A, G, I, S, T, M, or V; L332 replaced with A, G, I, S, T, M, or V; V333 replaced with A, G, I, L, S, T, or M; V335 replaced with A, G, I, L, S, T, or M; N336 replaced with Q; D337 replaced with E; A338 replaced with G, I, L, S, T, M, or V; D339 replaced with E; S340 replaced with A, G, I, L, T, M, or V; A341 replaced with G, I, L, S, T, M, or V; D342 replaced with E; I343 replaced with A, G, L, S, T, M, or V; S344 replaced with A, G, I, L, T, M, or V; T345 replaced with A, G, I, L, S, M, or V; L346 replaced with A, G, I, S, T, M, or V; L347 replaced with A, G, I, S, T, M, or V; D348 replaced with E; A349 replaced with G, I, L, S, T, M, or V; S350 replaced with A, G, I, L, T, M, or V; A351 replaced with G, I, L, S, T, M, or V; T352 replaced with A, G, I, L, S, M, or V; L353 replaced with A, G, I, S, T, M, or V; E354 replaced with D; E355 replaced with D; G356 replaced with A, I, L, S, T, M, or V; H357 replaced with K, or R; A358 replaced with G, I, L, S, T, M, or V; K359 replaced with H, or R; E360 replaced with D; T361 replaced with A, G, I, L, S, M, or V; I362 replaced with A, G, L, S, T, M, or V; Q363 replaced with N; D364 replaced with E; Q365 replaced with N; L366 replaced with A, G, I, S, T, M, or V; V367 replaced with A, G, I, L, S, T, or M; G368 replaced with A, I, L, S, T, M, or V; S369 replaced with A, G, I, L, T, M, or V; E370 replaced with D; K371 replaced with H, or R; L372 replaced with A, G, I, S, T, M, or V; F373 replaced with W, or Y; Y374 replaced with F, or W; E375 replaced with D; E376 replaced with D; D377 replaced with E; E378 replaced with D; A379 replaced with G, I, L, S, T, M, or V; G380 replaced with A, I, L, S, T, M, or V; S381 replaced with A, G, I, L, T, M, or V; A382 replaced with G, I, L, S, T, M, or V; T383 replaced with A, G, I, L, S, M, or V; S384 replaced with A, G, I, L, T, M, or V; and/or L386 replaced with A, G, I, S, T, M, or V of SEQ ID NO:4.

In specific embodiments, the antibodies of the invention bind TR10 polypeptides or fragments or variants thereof (especially a fragment comprising or alternatively consisting of, the extracellular soluble domain of TR10), that contains any one or more of the following non-conservative mutations in TR10: M1 replaced with D, E, H, K, R, N, Q, F, W, Y, P, or C; G2 replaced with D, E, H, K, R, N, Q, F, W, Y, P, or C; L3 replaced with D, E, H, K, R, N, Q, F, W, Y, P, or C; W4 replaced with D, E, H, K, R, N, Q, A, G, I, L, S, T, M, V, P, or C; G5 replaced with D, E, H, K, R, N, Q, F, W, Y, P, or C; Q6 replaced with D, E, H, K, R, A, G, I, L, S, T, M, V, F, W, Y, P, or C; S7 replaced with D, E, H, K, R, N, Q, F, W, Y, P, or C; V8 replaced with D, E, H, K, R, N, Q, F, W, Y, P, or C; P9 replaced with D, E, H, K, R, A, G, I, L, S, T, M, V, N, Q, F, W, Y, or C; T10 replaced with D, E, H, K, R, N, Q, F, W, Y, P, or C; A11 replaced with D, E, H, K, R, N, Q, F, W, Y, P, or C; S12 replaced with D, E, H, K, R, N, Q, F, W, Y, P, or C; S13 replaced with D, E, H, K, R, N, Q, F, W, Y, P, or C; A14 replaced with D, E, H, K, R, N, Q, F, W, Y, P, or C; R15 replaced with D, E, A, G, I, L, S, T, M, V, N, Q, F, W, Y, P, or C; A16 replaced with D, E, H, K, R, N, Q, F, W, Y, P, or C; G17 replaced with D, E, H, K, R, N, Q, F, W, Y, P, or C; R18 replaced with D, E, A, G, I, L, S, T, M, V, N, Q, F, W, Y, P, or C; Y19 replaced with D, E, H, K, R, N, Q, A, G, I, L, S, T, M, V, P, or C; P20 replaced with D, E, H, K, R, A, G, I, L, S, T, M, V, N, Q, F, W, Y, or C; G21 replaced with D, E, H, K, R, N, Q, F, W, Y, P, or C; A22 replaced with D, E, H, K, R, N, Q, F, W, Y, P, or C; R23 replaced with D, E, A, G, I, L, S, T, M, V, N, Q, F, W, Y, P, or C; T24 replaced with D, E, H, K, R, N, Q, F, W, Y, P, or C; A25 replaced with D, E, H, K, R, N, Q, F, W, Y, P, or C; S26 replaced with D, E, H, K, R, N, Q, F, W, Y, P, or C; G27 replaced with D, E, H, K, R, N, Q, F, W, Y, P, or C; T28 replaced with D, E, H, K, R, N, Q, F, W, Y, P, or C; R29 replaced with D, E, A, G, I, L, S, T, M, V, N, Q, F, W, Y, P, or C; P30 replaced with D, E, H, K, R, A, G, I, L, S, T, M, V, N, Q, F, W, Y, or C; W31 replaced with D, E, H, K, R, N, Q, A, I, L, S, T, M, V, P, or C; L32 replaced with D, E, H, K, K, R, N, Q, F, W, Y, P, or C; L33 replaced with D, E, H, K, R, N, Q, F, W, Y, P, or C; D34 replaced with H, K, R, A, G, I, L, S, T, M, V, N, Q, F, W, Y, P, or C; P35 replaced with D, E, H, K, R, A, G, I, L, S, T, M, V, N, Q, F, W, Y, or C; K36 replaced with D, E, A, G, I, L, S, T, M, V, N, Q, F, W, Y, P, or C; B7 replaced with D, E, H, K, R, N, Q, F, W, Y, P, or C; L38 replaced with D, E, H, K, R, N, Q, F, W, Y, P, or C; K39 replaced with D, E, A, G, I, L, S, T, M, V, N, Q, F, W, Y, P, or C; F40 replaced with D, E, H, K, R, N, Q, A, G, I, L, S, T, M, V, P, or C; V41 replaced with D, E, H, K, R, N, Q, F, W, Y, P, or C; V42 replaced with D, E, H, K, R, N, Q, F, W, Y, P, or C; F43 replaced with D, E, H, K, R, N, Q, A, G, I, L, S, T, M, V, P, or C; I44 replaced with D, E, H, K, R, N, Q, F, W, Y, P, or C; V45 replaced with D, E, H, K, R, N, Q, F, W, Y, P, or C; A46 replaced with D, E, H, K, R, N, Q, F, W, Y, P, or C; V47 replaced with D, E, H, K, R, N, Q, F, W, Y, P, or C; L48 replaced with D, E, H, K, R, N, Q, F, W, Y, P, or C; L49 replaced with D, E, H, K, R, N, Q, F, W, Y, P, or C; P50 replaced with D, E, H, K, R, A, G, I, L, S, T, M, V, N, Q, F, W, Y, or C; V51 replaced with D, E, H, K, R, N, Q, F, W, Y, P, or C; R52 replaced with D, E, A, G, I, L, S, T, M, V, N, Q, F, W, Y, P, or C; V53 replaced with D, E, H, K, R, N, Q, F, W, Y, P, or C; D54 replaced with H, K, R, A, G, I, L, S, T, M, V, N, Q, F, W, Y, P, or C; S55 replaced with D, E, H, K, R, N, Q, F, W, Y, P, or C; A56 replaced with D, E, H, K, R, N, Q, F, W, Y, P, or C; T57 replaced with D, E, H, K, R, N, Q, F, W, Y, P, or C; I58 replaced with D, E, H, K, R, N, Q, F, W, Y, P, or C; P59 replaced with D, E, H, K, R, A, G, I, L, S, T, M, V, N, Q, F, W, Y, or C; R60 replaced with D, E, A, G, I, L, S, T, M, V, N, Q, F, W, Y, P, or C; Q61 replaced with D, E, H, K, R, A, G, I, L, S, T, M, V, F, W, Y, P, or C; D62 replaced with H, K, R, A, G, I, L, S, T, M, V, N, Q, F, W, Y, P, or C; E63 replaced with H, K, R, A, G, I, L, S, T, M, V, N, Q, F, W, Y, P, or C; V64 replaced with D, E, H, K, R, N, Q, F, W, Y, P, or C; P65 replaced with D, E, H, K, R, A, G, I, L, S, T, M, V, N, Q, F, W, Y, or C; Q66 replaced with D, E, H, K, R, A, G, I, L, S, T, M, V, F, W, Y, P, or C; Q67 replaced with D, E, H, K, R, A, G, I, L, S, T, M, V, F, W, Y, P, or C; T68 replaced with D, E, H, K, R, N, Q, F, W, Y, P, or C; V69 replaced with D, E, H, K, R, N, Q, F, W, Y, P, or C; A70 replaced with D, E, H, K, R, N, Q, F, W, Y, P, or C; P71 replaced with D, E, H, K, R, A, G, I, L, S, T, M, V, N, Q, F, W, Y, or C; Q72 replaced with D, E, H, K, R, A, G, I, L, S, T, M, V, F, W, Y, P, or C; Q73 replaced with D, E, H, K, R, A, G, I, L, S, T, M, V, F, W, Y, P, or C; Q74 replaced with D, E, H, K, R, A, G, I, L, S, T, M, V, F, W, Y, P, or C; R75 replaced with D, E, A, G, I, L, S, T, M, V, N, Q, F, W, Y, P, or C; R76 replaced with D, E, A, G, I, L, S, T, M, V, N, Q, F, W, Y, P, or C; S77 replaced with D, E, H, K, R, N, Q, F, W, Y, P, or C; L78 replaced with D, E, H, K, R, N, Q, F, W, Y, P, or C; K79 replaced with D, E, A, G, I, L, S, T, M, V, N, Q, F, W, Y, P, or C; E80 replaced with H, K, R, A, G, I, L, S, T, M, V, N, Q, F, W, Y, P, or C; E81 replaced with H, K, R, A, G, I, L, S, T, M, V, N, Q, F, W, Y, P, or C; E82 replaced with H, K, R, A, G, I, L, S, T, M, V, N, Q, F, W, Y, P, or C; C83 replaced with D, E, H, K, R, A, G, I, L, S, T, M, V, N, Q, F, W, Y, or P; P84 replaced with D, E, H, K, R, A, G, I, L, S, T, M, V, N, Q, F, W, Y, or C; A85 replaced with D, E, H, K, R, N, Q, F, W, Y, P, or C; G86 replaced with D, E, H, K, R, N, Q, F, W, Y, P, or C; S87 replaced with D, E, H, K, R, N, Q, F, W, Y, P, or C; H88 replaced with D, E, A, G, I, L, S, T, M, V, N, Q, F, W, Y, P, or C; R89 replaced with D, E, A, G, I, L, S, T, M, V, N, Q, F, W, Y, P, or C; S90 replaced with D, E, H, K, R, N, Q, F, W, Y, P, or C; E91 replaced with H, K, R, A, G, I, L, S, T, M, V, N, Q, F, W, Y, P, or C; Y92 replaced with D, E, H, K, R, N, Q, A, G, I, L, S, T, M, V, P, or C; T93 replaced with D, E, H, K, R, N, Q, F, W, Y, P, or C; G94 replaced with D, E, H, K, R, N, Q, F, W, Y, P, or C; A95 replaced with D, E, H, K, R, N, Q, F, W, Y, P, or C; C96 replaced with D, E, H, K, R, A, G, I, L, S, T, M, V, N, Q, F, W, Y, or P; N97 replaced with D, E, H, K, R, A, G, I, L, S, T, M, V, F, W, Y, P, or C; P98 replaced with D, E, H, K, R, A, G, I, L, S, T, M, V, N, Q, F, W, Y, or C; C99 replaced with D, E, H, K, R, A, G, I, L, S, T, M, V, N, Q, F, W, Y, or P; T100 replaced with D, E, H, K, R, N, Q, F, W, Y, P, or C; E101 replaced with H, K, R, A, G, I, L, S, T, M, V, N, Q, F, W, Y, P, or C; G102 replaced with D, E, H, K, R, N, Q, F, W, Y, P, or C; V103 replaced with D, E, H, K, R, N, Q, F, W, Y, P, or C; D104 replaced with H, K, R, A, G, I, L, S, T, M, V, N, Q, F, W, Y, P, or C; Y105 replaced with D, E, H, K, R, N, Q, A, G, I, L, S, T, M, V, P, or C; T106 replaced with D, E, H, K, R, N, Q, F, W, Y, P, or C; I107 replaced with D, E, H, K, R, N, Q, F, W, Y, P, or C; A108 replaced with D, E, H, K, R, N, Q, F, W, Y, P, or C; S109 replaced with D, E, H, K, R, N, Q, F, W, Y, P, or C; N110 replaced with D, E, H, K, R, A, G, I, L, S, T, M, V, F, W, Y, P, or C; N111 replaced with D, E, H, K, R, A, G, I, L, S, T, M, V, F, W, Y, P, or C; L112 replaced with D, E, H, K, R, N, Q, F, W, Y, P, or C; P113 replaced with D, E, H, K, R, A, G, I, L, S, T, M, V, N, Q, F, W, Y, or C; S114 replaced with D, E, H, K, R, N, Q, F, W, Y, P, or C; C115 replaced with D, E, H, K, R, A, G, I, L, S, T, M, V, N, Q, F, W, Y, or P; L116 replaced with D, E, H, K, R, N, Q, F, W, Y, P, or C; L117 replaced with D, E, H, K, R, N, Q, F, W, Y, P, or C; C118 replaced with D, E, H, K, R, A, G, I, L, S, T, M, V, N, Q, F, W, Y, or P; T119 replaced with D, E, H, K, R, N, Q, F, W, Y, P, or C; V120 replaced with D, E, H, K, R, N, Q, F, W, Y, P, or C; C121 replaced with D, E, H, K, R, A, G, I, L, S, T, M, V, N, Q, F, W, Y, or P; K122 replaced with D, E, A, G, I, L, S, T, M, V, N, Q, F, W, Y, P, or C; S123 replaced with D, E, H, K, R, N, Q, F, W, Y, P, or C; G124 replaced with D, E, H, K, R, N, Q, F, W, Y, P, or C; Q125 replaced with D, E, H, K, R, A, G, I, L, S, T, M, V, F, W, Y, P, or C; T126 replaced with D, E, H, K, R, N, Q, F, W, Y, P, or C; N127 replaced with D, E, H, K, R, A, G, I, L, S, T, M, V, F, W, Y, P, or C; K128 replaced with D, E, A, G, I, L, S, T, M, V, N, Q, F, W, Y, P, or C; S129 replaced with D, E, H, K, R, N, Q, F, W, Y, P, or C; S130 replaced with D, E, H, K, R, N, Q, F, W, Y, P, or C; C131 replaced with D, E, H, K, R, A, G, I, L, S, T, M, V, N, Q, F, W, Y, or P; T132 replaced with D, E, H, K, R, N, Q, F, W, Y, P, or C; T133 replaced with D, E, H, K, R, N, Q, F, W, Y, P, or C; T134 replaced with D, E, H, K, R, N, Q, F, W, Y, P, or C; R135 replaced with D, E, A, G, I, L, S, T, M, V, N, Q, F, W, Y, P, or C; D136 replaced with H, K, R, A, G, I, L, S, T, M, V, N, Q, F, W, Y, P, or C; T137 replaced with D, E, H, K, R, N, Q, F, W, Y, P, or C; V138 replaced with D, E, H, K, R, N, Q, F, W, Y, P, or C; C139 replaced with D, E, H, K, R, A, G, I, L, S, T, M, V, N, Q, F, W, Y, or P; Q140 replaced with D, E, H, K, R, A, G, I, L, S, T, M, V, F, W, Y, P, or C; C141 replaced with D, E, H, K, R, A, G, I, L, S, T, M, V, N, Q, F, W, Y, or P; E142 replaced with H, K, R, A, G, I, L, S, T, M, V, N, Q, F, W, Y, P, or C; K143 replaced with D, E, A, G, I, L, S, T, M, V, N, Q, F, W, Y, P, or C; G144 replaced with D, E, H, K, R, N, Q, F, W, Y, P, or C; S145 replaced with D, E, H, K, R, N, Q, F, W, Y, P, or C; F146 replaced with D, E, H, K, R, N, Q, A, G, I, L, S, T, M, V, P, or C; Q147 replaced with D, E, H, K, R, A, G, I, L, S, T, M, V, F, W, Y, P, or C; D148 replaced with H, K, R, A, G, I, L, S, T, M, V, N, Q, F, W, Y, P, or C; K149 replaced with D, E, A, G, I, L, S, T, M, V, N, Q, F, W, Y, P, or C; N150 replaced with D, E, H, K, R, A, G, I, L, S, T, M, V, F, W, Y, P, or C; S151 replaced with D, E, H, K, R, N, Q, F, W, Y, P, or C; P152 replaced with D, E, H, K, R, A, G, I, L, S, T, M, V, N, Q, F, W, Y, or C; E153 replaced with H, K, R, A, G, I, L, S, T, M, V, N, Q, F, W, Y, P, or C; M154 replaced with D, E, H, K, R, N, Q, F, W, Y, P, or C; C155 replaced with D, E, H, K, R, A, G, I, L, S, T, M, V, N, Q, F, W, Y, or P; R156 replaced with D, E, A, G, I, L, S, T, M, V, N, Q, F, W, Y, P, or C; T157 replaced with D, E, H, K, R, N, Q, F, W, Y, P, or C; C158 replaced with D, E, H, K, R, A, G, I, L, S, T, M, V, N, Q, F, W, Y, or P; R159 replaced with D, E, A, G, I, L, S, T, M, V, N, Q, F, W, Y, P, or C; T160 replaced with D, E, H, K, R, N, Q, F, W, Y, P, or C; G161 replaced with D, E, H, K, R, N, Q, F, W, Y, P, or C; C162 replaced with D, E, H, K, R, A, G, I, L, S, T, M, V, N, Q, F, W, Y, or P; P163 replaced with D, E, H, K, R, A, G, I, L, S, T, M, V, N, Q, F, W, Y, or C; R164 replaced with D, E, A, G, I, L, S, T, M, V, N, Q, F, W, Y, P, or C; G165 replaced with D, E, H, K, R, N, Q, F, W, Y, P, or C; M166 replaced with D, E, H, K, R, N, Q, F, W, Y, P, or C; V167 replaced with D, E, H, K, R, N, Q, F, W, Y, P, or C; K168 replaced with D, E, A, G, I, L, S, T, M, V, N, Q, F, W, Y, P, or C; V169 replaced with D, E, H, K, R, N, Q, F, W, Y, P, or C; S170 replaced with D, E, H, K, R, N, Q, F, W, Y, P, or C; N171 replaced with D, E, H, K, R, A, G, I, L, S, T, M, V, F, W, Y, P, or C; C172 replaced with D, E, H, K, R, A, G, I, L, S, T, M, V, N, Q, F, W, Y, or P; T173 replaced with D, E, F, K, R, N, Q, F, W, Y, P, or C; P174 replaced with D, E, K, K, R, A, G, I, L, S, T, N, V, N, Q, F, W, Y, or C; R175 replaced with D, E, A, G, I, L, S, T, M, V, N, Q, F, W, Y, P, or C; S176 replaced with D, E, H, K, R, N, Q, F, W, Y, P, or C; D177 replaced with H, K, R, A, G, I, L, S, T, M, V, N, Q, F, W, Y, P, or C; I178 replaced with D, E, H, K, R, N, Q, F, W, Y, P, or C; K179 replaced with D, E, A, G, I, L, S, T, M, V, N, Q, F, W, Y, P, or C; C180 replaced with D, E, H, K, R, A, G, I, L, S, T, M, V, N, Q, F, W, Y, or P; K181 replaced with D, E, A, G, I, L, S, T, M, V, N, Q, F, W, Y, P, or C; N182 replaced with D, E, H, K, R, A, G, I, L, S, T, M, V, F, W, Y, P, or C; E183 replaced with H, K, R, A, G, I, L, S, T, M, V, N, Q, F, W, Y, P, or C; S184 replaced with D, E, K, K, R, N, Q, F, W, Y, P, or C; A185 replaced with D, E, H, K, R, N, Q, F, W, Y, P, or C; A186 replaced with D, E, H, K, R, N, Q, F, W, Y, P, or C; S187 replaced with D, E, H, K, R, N, Q, F, W, Y, P, or C; S188 replaced with D, E, H, K, R, N, Q, F, W, Y, P, or C; T189 replaced with D, E, H, K, R, N, Q, F, W, Y, P, or C; G190 replaced with D, E, H, K, R, N, Q, F, W, Y, P, or C; K191 replaced with D, E, A, G, I, L, S, T, M, V, N, Q, F, W, Y, P, or C; T192 replaced with D, E, H, K, R, N, Q, F, W, Y, P, or C; P193 replaced with D, E, H, K, R, A, G, I, L, S, T, M, V, N, Q, F, W, Y, or C; A194 replaced with D, E, H, K, R, N, Q, F, W, Y, P, or C; A195 replaced with D, E, H, K, R, N, Q, F, W, Y, P, or C; E196 replaced with H, K, R, A, G, I, L, S, T, M, V, N, Q, F, W, Y, P, or C; E197 replaced with H, K, R, A, G, I, L, S, T, M, V, N, Q, F, W, Y, P, or C; T198 replaced with D, E, H, K, R, N, Q, F, W, Y, P, or C; V199 replaced with D, E, H, K, R, N, Q, F, W, Y, P, or C; T200 replaced with D, E, H, K, R, N, Q, F, W, Y, P, or C; T201 replaced with D, E, H, K, R, N, Q, F, W, Y, P, or C; I202 replaced with D, E, H, K, R, N, Q, F, W, Y, P, or C; L203 replaced with D, E, H, K, R, N, Q, F, W, Y, P, or C; G204 replaced with D, E, H, K, R, N, Q, F, W, Y, P, or C; M205 replaced with D, E, H, K, R, N, Q, F, W, Y, P, or C; L206 replaced with D, E, H, K, R, N, Q, F, W, Y, P, or C; A207 replaced with D, E, H, K, R, N, Q, F, W, Y, P, or C; S208 replaced with D, E, H, K, R, N, Q, F, W, Y, P, or C; P209 replaced with D, E, H, K, R, A, G, I, L, S, T, M, V, N, Q, F, W, Y, or C; Y210 replaced with D, E, H, K, R, N, Q, A, G, I, L, S, T, M, V, P, or C; H211 replaced with D, E, A, G, I, L, S, T, M, V, N, Q, F, W, Y, P, or C; Y212 replaced with D, E, H, K, R, N, Q, A, G, I, L, S, T, M, V, P, or C; L213 replaced with D, E, H, K, R, N, Q, F, W, Y, P, or C; I214 replaced with D, E, H, K, I, N, Q, F, W, Y, P, or C; I215 replaced with D, E, H, K, R, N, Q, F, W, Y, P, or C; I216 replaced with D, E, H, K, I, N, Q, F, W, Y, P, or C; V217 replaced with D, E, H, K, R, N, Q, F, W, Y, P, or C; V218 replaced with D, E, H, K, R, N, Q, F, W, Y, P, or C; L219 replaced with D, E, H, K, R, N, Q, F, W, Y, P, or C; V220 replaced with D, E, H, K, R, N, Q, F, W, Y, P, or C; I221 replaced with D, E, H, K, R, N, Q, F, W, Y, P, or C; I222 replaced with D, E, H, K, R, N, Q, F, W, Y, P, or C; L223 replaced with D, E, H, K, R, N, Q, F, W, Y, P, or C; A224 replaced with D, E, H, K, R, N, Q, F, W, Y, P, or C; V225 replaced with D, E, H, K, R, N, Q, F, W, Y, P, or C; V226 replaced with D, E, H, K, R, N, Q, F, W, Y, P, or C; V227 replaced with D, E, H, K, R, N, Q, F, W, Y, P, or C; V228 replaced with D, E, H, K, R, N, Q, F, W, Y, P, or C; G229 replaced with D, E, H, K, R, N, Q, F, W, Y, P, or C; F230 replaced with D, E, H, K, R, N, Q, A, G, I, L, S, T, M, V, P, or C; S231 replaced with D, E, H, K, R, N, Q, F, W, Y, P, or C; C232 replaced with D, E, H, K, R, A, G, I, L, S, T, M, V, N, Q, F, W, Y, or P; R233 replaced with D, E, A, G, I, L, S, T, M, V, N, Q, F, W, Y, P, or C; K234 replaced with D, E, A, G, I, L, S, T, M, V, N, Q, F, W, Y, P, or C; K235 replaced with D, E, A, G, I, L, S, T, M, V, N, Q, F, W, Y, P, or C; F236 replaced with D, E, H, K, R, N, Q, A, G, I, L, S, T, M, V, P, or C; I237 replaced with D, E, H, K, R, N, Q, F, W, Y, P, or C; S238 replaced with D, E, H, K, R, N, Q, F, W, Y, P, or C; Y239 replaced with D, E, H, K, R, N, Q, A, G, I, L, S, T, M, V, P, or C; L240 replaced with D, E, H, K, R, N, Q, F, W, Y, P, or C; K241 replaced with D, E, A, G, I, L, S, T, M, V, N, Q, F, W, Y, P, or C; G242 replaced with D, E, H, K, R, N, Q, F, W, Y, P, or C; I243 replaced with D, E, H, K, R, N, Q, F, W, Y, P, or C; C244 replaced with D, E, H, K, R, A, G, I, L, S, T, M, V, N, Q, F, W, Y, or P; S245 replaced with D, E, H, K, R, N, Q, F, W, Y, P, or C; G246 replaced with D, E, H, K, R, N, Q, F, W, Y, P, or C; G247 replaced with D, E, H, K, R, N, Q, F, W, Y, P, or C; G248 replaced with D, E, H, K, R, N, Q, F, W, Y, P, or C; G249 replaced with D, E, H, K, R, N, Q, F, W, Y, P, or C; G250 replaced with D, E, H, K, R, N, Q, F, W, Y, P, or C; P251 replaced with D, E, H, K, R, A, G, I, L, S, T, M, V, N, Q, F, W, Y, or C; E252 replaced with H, K, R, A, G, I, L, S, T, M, V, N, Q, F, W, Y, P, or C; R253 replaced with D, E, A, G, I, L, S, T, M, V, N, Q, F, W, Y, P, or C; V254 replaced with D, E, F, K, R, N, Q, F, W, Y, P, or C; H255 replaced with D, E, A, G, I, L, S, T, M, V, N, Q, F, W, Y, P, or C; R256 replaced with D, E, A, G, I, L, S, T, M, V, N, Q, F, W, Y, P, or C; V257 replaced with D, E, H, K, R, N, Q, F, W, Y, P, or C; L258 replaced with D, E, H, K, R, N, Q, F, W, Y, P, or C; F259 replaced with D, E, H, K, R, N, Q, A, I, L, S, T, M, V, P, or C; R260 replaced with D, E, A, G, I, L, S, T, M, V, N, Q, F, W, Y, P, or C; R261 replaced with D, E, A, G, I, L, S, T, M, V, N, Q, F, W, Y, P, or C; R262 replaced with D, E, A, G, I, L, S, T, M, V, N, Q, F, W, Y, P, or C; S263 replaced with D, E, H, K, R, N, Q, F, W, Y, P, or C; C264 replaced with D, E, H, K, R, A, G, I, L, S, T, M, V, N, Q, F, W, Y, or P; P265 replaced with D, E, H, K, R, A, G, I, L, S, T, M, V, N, Q, F, W, Y, or C; S266 replaced with D, E, H, K, R, N, Q, F, W, Y, P, or C; R267 replaced with D, E, A, G, I, L, S, T, M, V, N, Q, F, W, Y, P, or C; V268 replaced with D, E, H, K, R, N, Q, F, W, Y, P, or C; P269 replaced with D, E, H, K, R, A, G, I, L, S, T, M, V, N, Q, F, W, Y, or C; G270 replaced with D, E, H, K, R, N, Q, F, W, Y, P, or C; A271 replaced with D, E, H, K, R, N, Q, F, W, Y, P, or C; E272 replaced with H, K, R, A, G, I, L, S, T, M, V, N, Q, F, W, Y, P, or C; D273 replaced with H, K, R, A, G, I, L, S, T, M, V, N, Q, F, W, Y, P, or C; N274 replaced with D, E, H, K, R, A, G, I, L, S, T, M, V, F, W, Y, P, or C; A275 replaced with D, E, H, K, R, N, Q, F, W, Y, P, or C; R276 replaced with D, E, A, G, I, L, S, T, M, V, N, Q, F, W, Y, P, or C; N277 replaced with D, E, H, K, R, A, G, I, L, S, T, M, V, F, W, Y, P, or C; E278 replaced with H, K, R, A, G, I, L, S, T, M, V, N, Q, F, W, Y, P, or C; T279 replaced with D, E, H, K, R, N, Q, F, W, Y, P, or C; L280 replaced with D, E, H, K, R, N, Q, F, W, Y, P, or C; S281 replaced with D, E, H, K, R, N, Q, F, W, Y, P, or C; N282 replaced with D, E, H, K, R, A, G, I, L, S, T, M, V, F, W, Y, P, or C; R283 replaced with D, E, A, G, I, L, S, T, M, V, N, Q, F, W, Y, P, or C; Y284 replaced with D, E, H, K, R, N, Q, A, G, I, L, S, T, M, V, P, or C; L285 replaced with D, E, H, K, R, N, Q, F, W, Y, P, or C; Q286 replaced with D, E, H, K, R, A, G, I, L, S, T, M, V, F, W, Y, P, or C; P287 replaced with D, E, H, K, R, A, G, I, L, S, T, M, V, N, Q, F, W, Y, or C; T288 replaced with D, E, H, K, R, N, Q, F, W, Y, P, or C; Q289 replaced with D, E, H, K, R, A, G, I, L, S, T, M, V, F, W, Y, P, or C; V290 replaced with D, E, H, K, R, N, Q, F, W, Y, P, or C; S291 replaced with D, E, H, K, R, N, Q, F, W, Y, P, or C; E292 replaced with H, K, R, A, G, I, L, S, T, M, V, N, Q, F, W, Y, P, or C; Q293 replaced with D, E, H, K, R, A, G, I, L, S, T, M, V, F, W, Y, P, or C; E294 replaced with H, K, R, A, G, I, L, S, T, M, V, N, Q, F, W, Y, P, or C; I295 replaced with D, E, H, K, R, N, Q, F, W, Y, P, or C; Q296 replaced with D, E, H, K, R, A, G, I, L, S, T, M, V, F, W, Y, P, or C; G297 replaced with D, E, H, K, R, N, Q, F, W, Y, P, or C; Q298 replaced with D, E, H, K, R, A, G, I, L, S, T, V, F, W, Y, P, or C; E299 replaced with H, K, R, A, G, I, L, S, T, M, V, N, Q, F, W, Y, P, or C; L300 replaced with D, E, H, K, R, N, Q, F, W, Y, P, or C; A301 replaced with D, E, H, K, R, N, Q, F, W, Y, P, or C; E302 replaced with H, K, R, A, G, I, L, S, T, M, V, N, Q, F, W, Y, P, or C; L303 replaced with D, E, H, K, R, N, Q, F, W, Y, P, or C; T304 replaced with D, E, H, K, R, N, Q, F, W, Y, P, or C; G305 replaced with D, E, H, K, R, N, Q, F, W, Y, P, or C; V306 replaced with D, E, H, K, R, N, Q, F, W, Y, P, or C; T307 replaced with D, E, H, K, R, N, Q, F, W, Y, P, or C; V308 replaced with D, E, H, K, R, N, Q, F, W, Y, P, or C; E309 replaced with H, K, R, A, G, I, L, S, T, M, V, N, Q, F, W, Y, P, or C; S310 replaced with D, E, H, K, R, N, Q, F, W, Y, P, or C; P311 replaced with D, E, H, K, R, A, G, I, L, S, T, M, V, N, Q, F, W, Y, or C; E312 replaced with H, K, R, A, G, I, L, S, T, M, V, N, Q, F, W, Y, P, or C; E313 replaced with H, K, R, A, G, I, L, S, T, M, V, N, Q, F, W, Y, P, or C; P314 replaced with D, E, H, K, R, A, G, I, L, S, T, M, V, N, Q, F, W, Y, or C; Q315 replaced with D, E, H, K, R, A, G, I, L, S, T, M, V, F, W, Y, P, or C; R316 replaced with D, E, A, G, I, L, S, T, M, V, N, Q, F, W, Y, P, or C; L317 replaced with D, E, H, K, R, N, Q, F, W, Y, P, or C; L318 replaced with D, E, H, K, R, N, Q, F, W, Y, P, or C; E319 replaced with H, K, R, A, G, I, L, S, T, M, V, N, Q, F, W, Y, P, or C; Q320 replaced with D, E, H, K, R, A, G, I, L, S, T, M, V, F, W, Y, P, or C; A321 replaced with D, E, H, K, R, N, Q, F, W, Y, P, or C; E322 replaced with H, K, R, A, G, I, L, S, T, M, V, N, Q, F, W, Y, P, or C; A323 replaced with D, E, H, K, R, N, Q, F, W, Y, P, or C; E324 replaced with H, K, R, A, G, I, L, S, T, M, V, N, Q, F, W, Y, P, or C; G325 replaced with D, E, H, K, R, N, Q, F, W, Y, P, or C; C326 replaced with D, E, H, K, R, A, G, I, L, S, T, M, V, N, Q, F, W, Y, or P; Q327 replaced with D, E, H, K, R, A, G, I, L, S, T, M, V, F, W, Y, P, or C; R328 replaced with D, E, A, G, I, L, S, T, M, V, N, Q, F, W, Y, P, or C; R329 replaced with D, E, A, G, I, L, S, T, M, V, N, Q, F, W, Y, P, or C; R330 replaced with D, E, A, G, I, L, S, T, M, V, N, Q, F, W, Y, P, or C; L331 replaced with D, E, H, K, R, N, Q, F, W, Y, P, or C; L332 replaced with D, E, H, K, R, N, Q, F, W, Y, P, or C; V333 replaced with D, E, H, K, R, N, Q, F, W, Y, P, or C; P334 replaced with D, E, H, K, R, A, G, I, L, S, T, M, V, N, Q, F, W, Y, or C; V335 replaced with D, E, H, K, R, N, Q, F, W, Y, P, or C; N336 replaced with D, E, H, K, R, A, G, I, L, S, T, M, V, F, W, Y, P, or C; D337 replaced with H, K, R, A, G, I, L, S, T, M, V, N, Q, F, W, Y, P, or C; A338 replaced with D, E, H, K, R, N, Q, F, W, Y, P, or C; D339 replaced with H, K, R, A, G, I, L, S, T, M, V, N, Q, F, W, Y, P, or C; S340 replaced with D, E, H, K, R, N, Q, F, W, Y, P, or C; A341 replaced with D, E, H, K, R, N, Q, F, W, Y, P, or C; D342 replaced with H, K, R, A, G, I, L, S, T, M, V, N, Q, F, W, Y, P, or C; I343 replaced with D, E, H, K, R, N, Q, F, W, Y, P, or C; S344 replaced with D, E, H, K, R, N, Q, F, W, Y, P, or C; T345 replaced with D, E, H, K, R, N, Q, F, W, Y, P, or C; L346 replaced with D, E, H, K, R, N, Q, F, W, Y, P, or C; L347 replaced with D, E, H, K, R, N, Q, F, W, Y, P, or C; D348 replaced with H, K, R, A, G, I, L, S, T, M, V, N, Q, F, W, Y, P, or C; A349 replaced with D, E, H, K, R, N, Q, F, W, Y, P, or C; S350 replaced with D, E, H, K, R, N, Q, F, W, Y, P, or C; A351 replaced with D, E, H, K, R, N, Q, F, W, Y, P, or C; T352 replaced with D, E, H, K, R, N, Q, F, W, Y, P, or C; L353 replaced with D, E, H, K, R, N, Q, F, W, Y, P, or C; E354 replaced with H, K, R, A, G, I, L, S, T, M, V, N, Q, F, W, Y, P, or C; E355 replaced with H, K, R, A, G, I, L, S, T, M, V, N, Q, F, W, Y, P, or C; G356 replaced with D, E, H, K, R, N, Q, F, W, Y, P, or C; H357 replaced with D, E, A, G, I, L, S, T, M, V, N, Q, F, W, Y, P, or C; A358 replaced with D, E, H, K, R, N, Q, F, W, Y, P, or C; K359 replaced with D, E, A, G, I, L, S, T, M, V, N, Q, F, W, Y, P, or C; E360 replaced with H, K, R, A, G, I, L, S, T, M, V, N, Q, F, W, Y, P, or C; T361 replaced with D, E, H, K, R, N, Q, F, W, Y, P, or C; I362 replaced with D, E, H, K, R, N, Q, F, W, Y, P, or C; Q363 replaced with D, E, H, K, R, A, G, I, L, S, T, M, V, F, W, Y, P, or C; D364 replaced with H, K, R, A, G, I, L, S, T, M, V, N, Q, F, W, Y, P, or C; Q365 replaced with D, E, H, K, R, A, G, I, L, S, T, M, V, F, W, Y, P, or C; L366 replaced with D, E, H, K, R, N, Q, F, W, Y, P, or C; V367 replaced with D, E, H, K, R, N, Q, F, W, Y, P, or C; G368 replaced with D, E, H, K, R, N, Q, F, W, Y, P, or C; S369 replaced with D, E, H, K, R, N, Q, F, W, Y, P, or C; E370 replaced with H, K, R, A, G, I, L, S, T, M, V, N, Q, F, W, Y, P, or C; K371 replaced with D, E, A, G, I, L, S, T, M, V, N, Q, F, W, Y, P, or C; L372 replaced with D, E, H, K, R, N, Q, F, W, Y, P, or C; F373 replaced with D, E, H, K, R, N, Q, A, G, I, L, S, T, M, V, P, or C; Y374 replaced with D, E, H, K, R, N, Q, A, G, I, L, S, T, M, V, P, or C; E375 replaced with H, K, R, A, G, I, L, S, T, M, V, N, Q, F, W, Y, P, or C; E376 replaced with H, K, R, A, G, I, L, S, T, M, V, N, Q, F, W, Y, P, or C; D377 replaced with H, K, R, A, G, I, L, S, T, M, V, N, Q, F, W, Y, P, or C; E378 replaced with H, K, R, A, G, I, L, S, T, M, V, N, Q, F, W, Y, P, or C; A379 replaced with D, E, H, K, R, N, Q, F, W, Y, P, or C; G380 replaced with D, E, H, K, R, N, Q, F, W, Y, P, or C; S381 replaced with D, E, H, K, R, N, Q, F, W, Y, P, or C; A382 replaced with D, E, H, K, R, N, Q, F, W, Y, P, or C; T383 replaced with D, E, H, K, R, N, Q, F, W, Y, P, or C; S384 replaced with D, E, H, K, R, N, Q, F, W, Y, P, or C; C385 replaced with D, E, H, K, R, A, G, I, L, S, T, M, V, N, Q, F, W, Y, or P; and/or L386 replaced with D, E, H, K, R, N, Q, F, W, Y, P, or C of SEQ ID NO:4.

Amino acids in the TR10 protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)). In preferred embodiments, antibodies of the present invention bind regions of TR10 that are essential for TR10 function. In other preferred embodiments, antibodies of the present invention bind regions of TR10 that are essential for TR10 function and inhibit or abolish TR10 function. In other preferred embodiments, antibodies of the present invention bind regions of TR10 that are essential for TR10 function and enhance TR10 function.

To improve or alter the characteristics of TR10 polypeptides, protein engineering may be employed. Recombinant DNA technology known to those skilled in the art can be used to create novel mutant proteins or “muteins” including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions.

Non-naturally occurring TR10 variants that may be bound by the antibodies of the invention may be produced using art-known mutagenesis techniques, which include, but are not limited to oligonucleotide mediated mutagenesis, alanine scanning, PCR mutagenesis, site directed mutagenesis (see e.g., Carter et al., Nucl. Acids Res. 13:4331 (1986); and Zoller et al., Nucl. Acids Res. 10:6487 (1982)), cassette mutagenesis (see e.g., Wells et al., Gene 34:315 (1985)), restriction selection mutagenesis (see e.g., Wells et al., Philos. Trans. R Soc. London SerA 317:415 (1986)).

Thus, the invention also encompasses antibodies that bind TR10 derivatives and analogs that have one or more amino acid residues deleted, added, or substituted to generate TR10 polypeptides that are better suited for expression, scale up, etc., in the host cells chosen. For example, cysteine residues can be deleted or substituted with another amino acid residue in order to eliminate disulfide bridges; N-linked glycosylation sites can be altered or eliminated to achieve, for example, expression of a homogeneous product that is more easily recovered and purified from yeast hosts which are known to hyperglycosylate N-linked sites. To this end, a variety of amino acid substitutions at one or both of the first or third amino acid positions on any one or more of the glycosylation recognitions sequences in the TR10 polypeptides, and/or an amino acid deletion at the second position of any one or more such recognition sequences will prevent glycosylation of the TR10 at the modified tripeptide sequence (see, e.g., Miyajimo et al., EMBO J 5(6):1193-1197). Additionally, one or more of the amino acid residues of TR10 polypeptides (e.g., arginine and lysine residues) may be deleted or substituted with another residue to eliminate undesired processing by proteases such as, for example, furins or kexins.

The antibodies of the present invention also include antibodies that bind a polypeptide comprising, or alternatively, consisting of the polypeptide encoded by the deposited cDNA (the deposit having ATCC Accession Number 209040) including the leader; a polypeptide comprising, or alternatively, consisting of the mature polypeptide encoded by the deposited cDNA minus the leader (i.e., the mature protein); a polypeptide comprising, or alternatively, consisting of amino acids from about 1 to about 386 in SEQ ID NO:4; a polypeptide comprising, or alternatively, consisting of amino acids from about 2 to about 386 in SEQ ID NO:4; a polypeptide comprising, or alternatively, consisting of amino acids from about 56 to about 386 in SEQ ID NO:4; a polypeptide comprising, or alternatively, consisting of the extracellular domain; a polypeptide comprising, or alternatively, consisting of the cysteine rich domain; a polypeptide comprising, or alternatively, consisting of the transmembrane domain; a polypeptide comprising, or alternatively, consisting of the intracellular domain; a polypeptide comprising, or alternatively, consisting of the extracellular and intracellular domains with all or part of the transmembrane domain deleted; and a polypeptide comprising, or alternatively, consisting of the partial death domain; as well as polypeptides which are at least 80% identical, more preferably at least 80%, 85%, 90%, or 95% identical, still more preferably at least 96%, 97%, 98%, or 99% identical to the polypeptides described above (e.g., the polypeptide encoded by the deposited cDNA clone (the deposit having ATCC Accession Number 209040), the polypeptide of SEQ ID NO:4 (SEQ ID NO:4)), and also include portions of such polypeptides with at least 30 amino acids and more preferably at least 50 amino acids.

By a polypeptide having an amino acid sequence at least, for example, 95% “identical” to a reference amino acid sequence of a TR10 polypeptide is intended that the amino acid sequence of the polypeptide is identical to the reference sequence except that the polypeptide sequence may include up to five amino acid alterations per each 100 amino acids of the reference amino acid of the TR10 receptor. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98%, or 99% identical to, for instance, the amino acid sequence shown in SEQ ID NO:4, or to the amino acid sequence encoded by the deposited cDNA clone, can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In a specific embodiment, the identity between a reference (query) sequence (a sequence of the present invention) and a subject sequence, also referred to as a global sequence alignment, is determined using the FASTDB computer program based on the algorithm of Brutlag et al. (Comp. App. Biosci. 6:237-245 (1990)). Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty-0.05, Window Size=500 or the length of the subject amino acid sequence, whichever is shorter. According to this embodiment, if the subject sequence is shorter than the query sequence due to N- or C-terminal deletions, not because of internal deletions, a manual correction is made to the results to take into consideration the fact that the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. A determination of whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of this embodiment. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are made for the purposes of this embodiment.

The present application is also directed to antibodies that bind polypeptides at least 80%, 85%, 90%, 95%, 96%, 97%, 98% or 99% identical to the TR10 polypeptide sequence set forth as n⁷-m⁷, and/or n⁸-m⁸ herein. In preferred embodiments, the application is directed to proteins containing polypeptides at least 80%, 85%, 90%, 95%, 96%, 97%, 98% or 99% identical to polypeptides having the amino acid sequence of the specific TR10 N- and C-terminal deletions recited herein. Polynucleotides encoding these polypeptides are also encompassed by the invention.

In certain preferred embodiments, antibodies of the invention bind TR10 fusion proteins as described above wherein the TR10 portion of the fusion protein are those described as n⁷-m⁷, and/or n⁸-m⁸ herein.

Antibodies of the Invention May Bind Modified TRAIL Receptor Polypeptides

It is specifically contemplated that antibodies of the present invention may bind modified forms of TRAIL Receptor proteins (e.g., TR4, TR5, TR7, and/or TR10 (SEQ ID NOS:1-4, respectively)

In specific embodiments, antibodies of the present invention bind TRAIL receptor polypeptides (such as those described above) including, but not limited to naturally purified TRAIL receptor polypeptides, TRAIL receptor polypeptides produced by chemical synthetic procedures, and TRAIL receptor polypeptides produced by recombinant techniques from a prokaryotic or eukaryotic host, including, for example, bacterial, yeast, higher plant, insect and mammalian cells using, for example, the recombinant compositions and methods described above. Depending upon the host employed in a recombinant production procedure, the polypeptides may be glycosylated or non-glycosylated. In addition, TRAIL Receptor polypeptides may also include an initial modified methionine residue, in some cases as a result of host-mediated processes.

In addition, TRAIL Receptor proteins that are bound by antibodies of the present invention can be chemically synthesized using techniques known in the art (e.g., see Creighton, Proteins: Structures and Molecular Principles, W.H. Freeman & Co., N.Y. (1983), and Hunkapiller, et al., Nature 310:105-111 (1984)). For example, a peptide corresponding to a fragment of a TRAIL Receptor polypeptide can be synthesized by use of a peptide synthesizer. Furthermore, if desired, nonclassical amino acids or chemical amino acid analogs can be introduced as a substitution or addition into the TRAIL Receptor polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention additionally, encompasses antibodies that bind TRAIL Receptor polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin; etc.

Additional post-translational modifications to TRAIL Receptor polypeptides for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptides may also be modified with a detectable label, such as an enzymatic, fluorescent, isotopic or affinity label to allow for detection and isolation of the protein.

Also provided by the invention are antibodies that bind chemically modified derivatives of TRAIL Receptor polypeptide which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000,40,000, 50,000, 55,000, 60,000, 65,000, 70,000, 75,000, 80,000, 85,000, 90,000, 95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. For example, polyethylene glycol can be linked to a proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO95/06058; and WO98/32466, the disclosures of each of which are incorporated herein by reference.

One system for attaching polyethylene glycol directly to amino acid residues of proteins without an intervening linker employs tresylated MPEG, which is produced by the modification of monmethoxy polyethylene glycol (MPEG) using tresylchloride (CISO₂CH₂CF₃). Upon reaction of protein with tresylated MPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoreothane sulphonyl group.

Polyethylene glycol can also be attached to proteins using a number of different intervening linkers. For example, U.S. Pat. No. 5,612,460, the entire disclosure of which is incorporated herein by reference, discloses urethane linkers for connecting polyethylene glycol to proteins. Protein-polyethylene glycol conjugates wherein the polyethylene glycol is attached to the protein by a linker can also be produced by reaction of proteins with compounds such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number additional polyethylene glycol derivatives and reaction chemistries for attaching polyethylene glycol to proteins are described in WO 98/32466, the entire disclosure of which is incorporated herein by reference. Pegylated protein products produced using the reaction chemistries set out herein are included within the scope of the invention.

The number of polyethylene glycol moieties attached to each TRAIL Receptor polypeptide (i.e., the degree of substitution) may also vary. For example, the pegylated proteins of the invention may be linked, on average, to 1, 2, 3, 4, 5, 6, 7, 8, 9, 10, 12, 15, 17, 20, or more polyethylene glycol molecules. Similarly, the average degree of substitution within ranges such as 1-3, 24, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

As mentioned the antibodies of the present invention may bind TRAIL Receptor polypeptides that are modified by either natural processes, such as posttranslational processing, or by chemical modification techniques which are well known in the art. It will be appreciated that the same type of modification may be present in the same or varying degrees at several sites in a given TRAIL Receptor polypeptide. TRAIL Receptor polypeptides may be branched, for example, as a result of ubiquitination, and they may be cyclic, with or without branching. Cyclic, branched, and branched cyclic TRAIL Receptor polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

In one embodiment, the invention provides antibodies (each consisting of two heavy chains and two light chains linked together by disulfide bridges to form an antibody) that immunospecifically binds one or more TRAIL receptor polypeptides (e.g., SEQ ID NOS:1-4) or fragments or variants thereof, wherein the amino acid sequence of the heavy chain and the amino acid sequence of the light chain are the same as the amino acid sequence of a heavy chain and a light chain expressed by one or more cell lines referred to in Table 1. In another embodiment, the invention provides antibodies (each consisting of two heavy chains and two light chains linked together by disulfide bridges to form an antibody) that immunospecifically binds one or more TRAIL receptor polypeptides (e.g., SEQ ID NOS:1-4) or fragments or variants thereof, wherein the amino acid sequence of the heavy chain or the amino acid sequence of the light chain are the same as the amino acid sequence of a heavy chain or a light chain expressed by one or more cell lines referred to in Table 1. Immunospecific binding to TRAIL receptor polypeptides may be determined by immunoassays known in the art or described herein for assaying specific antibody-antigen binding. Molecules comprising, or alternatively consisting of, fragments or variants of these antibodies that immunospecifically bind to one or more TRAIL receptor are also encompassed by the invention, as are nucleic acid molecules encoding these antibodies molecules, fragments and/or variants.

In one embodiment of the present invention, antibodies that immunospecifically bind to a TRAIL receptor or a fragment or variant thereof, comprise a polypeptide having the amino acid sequence of any one of the heavy chains expressed by at least one of the cell lines referred to in Table 1 and/or any one of the light chains expressed by at least one of the cell lines referred to in Table 1. In another embodiment of the present invention, antibodies that immunospecifically bind to a TRAIL receptor or a fragment or variant thereof, comprise a polypeptide having the amino acid sequence of any one of the VH domains of a heavy chain expressed by at least one of the cell lines referred to in Table 1 and/or any one of the VL domains of a light chain expressed by at least one of the cell lines referred to in Table 1. In preferred embodiments, antibodies of the present invention comprise the amino acid sequence of a VH domain and VL domain expressed by the same cell line selected from the group consisting of cell lines referred to in Table 1. In alternative embodiments, antibodies of the present invention comprise the amino acid sequence of a VI domain and a VL domain from different cell lines referred to in Table 1. Molecules comprising, or alternatively consisting of, antibody fragments or variants of the VH and/or VL domains expressed by at least one of the cell lines referred to in Table 1 that immunospecifically bind to a TRAIL receptor are also encompassed by the invention, as are nucleic acid molecules encoding these VH and VL domains, molecules, fragments and/or variants.

The present invention also provides antibodies that immunospecificially bind to a polypeptide, or polypeptide fragment or variant of a TRAIL receptor, wherein said antibodies comprise, or alternatively consist of, a polypeptide having an amino acid sequence of any one, two, three, or more of the VH CDRs contained in a heavy chain expressed by one or more cell lines referred to in Table 1. In particular, the invention provides antibodies that immunospecifically bind a TRAIL receptor, comprising, or alternatively consisting of, a polypeptide having the amino acid sequence of a VH CDR1 contained in a heavy chain expressed by one or more cell lines referred to in Table 1. In another embodiment, antibodies that immunospecifically bind a TRAIL receptor, comprise, or alternatively consist of, a polypeptide having the amino acid sequence of a VH CDR2 contained in a heavy chain expressed by one or more cell lines referred to in Table 1. In a preferred embodiment, antibodies that immunospecifically bind a TRAIL receptor, comprise, or alternatively consist of a polypeptide having the amino acid sequence of a VH CDR3 contained in a heavy chain expressed by one or more cell lines referred to in Table 1. Molecules comprising, or alternatively consisting of, these antibodies, or antibody fragments or variants thereof, that immunospecifically bind to TRAIL receptor or a TRAIL receptor fragment or variant thereof are also encompassed by the invention, as are nucleic acid molecules encoding these antibodies, molecules, fragments and/or variants.

The present invention also provides antibodies that immunospecificially bind to a polypeptide, or polypeptide fragment or variant of a TRAIL receptor, wherein said antibodies comprise, or alternatively consist of, a polypeptide having an amino acid sequence of any one, two, three, or more of the VL CDRs contained in a light chain expressed by one or more cell lines referred to in Table 1. In particular, the invention provides antibodies that immunospecifically bind a TRAIL receptor, comprising, or alternatively consisting of, a polypeptide having the amino acid sequence of a VL CDR1 contained in a light chain expressed by one or more cell lines referred to in Table 1. In another embodiment, antibodies that immunospecifically bind a TRAIL receptor, comprise, or alternatively consist of, a polypeptide having the amino acid sequence of a VL CDR2 contained in a light chain expressed by one or more cell lines referred to in Table 1. In a preferred embodiment, antibodies that immunospecifically bind a TRAIL receptor, comprise, or alternatively consist of a polypeptide having the amino acid sequence of a VL CDR3 contained in a light chain expressed by one or more cell lines referred to in Table 1. Molecules comprising, or alternatively consisting of, these antibodies, or antibody fragments or variants thereof, that immunospecifically bind to TRAIL receptor or a TRAIL receptor fragment or variant thereof are also encompassed by the invention, as are nucleic acid molecules encoding these antibodies, molecules, fragments and/or variants.

The present invention also provides antibodies (including molecules comprising, or alternatively consisting of, antibody fragments or variants) that immunospecifically bind to a TRAIL receptor polypeptide or polypeptide fragment or variant of a TRAIL receptor, wherein said antibodies comprise, or alternatively consist of, one, two, three, or more VH CDRs and one, two, three or more VL CDRs, as contained in a heavy chain or light chain expressed by one or more cell lines referred to in Table 1. In particular, the invention provides for antibodies that immunospecifically bind to a polypeptide or polypeptide fragment or variant of a TRAIL receptor, wherein said antibodies comprise, or alternatively consist of, a VH CDR1 and a VL CDR1, a VH CDR1 and a VL CDR2, a VH CDR1 and a VL CDR3, a VR CDR2 and a VL CDR1, VH CDR2 and VL CDR2, a VH CDR2 and a VL CDR3, a VH CDR3 and a VH CDR1, a VH CDR3 and a VL CDR2, a VH CDR3 and a VL CDR3, or any combination thereof, of the VH CDRs and VL CDRs contained in a heavy chain or light chain expressed by one or more cell lines referred to in Table 1. In a preferred embodiment, one or more of these combinations are from the same antibody as disclosed in Table 1. Molecules comprising, or alternatively consisting of, fragments or variants of these antibodies, that immunospecifically bind to TRAIL receptor are also encompassed by the invention, as are nucleic acid molecules encoding these antibodies, molecules, fragments or variants.

Nucleic Acid Molecules Encoding Anti-TRAIL Receptor Antibodies

The present invention also provides for nucleic acid molecules, generally isolated, encoding an antibody of the invention (including molecules comprising, or alternatively consisting of, antibody fragments or variants thereof). In a specific embodiment, a nucleic acid molecule of the invention encodes an antibody (including molecules comprising, or alternatively consisting of, antibody fragments or variants thereof), comprising, or alternatively consisting of, a VH domain having an amino acid sequence of any one of the VH domains expressed by at least one of the cell lines referred to in Table 1 and a VL domain having an amino acid sequence of any one of the VL domains expressed by at least one of the cell lines referred to in Table 1. In another embodiment, a nucleic acid molecule of the invention encodes an antibody (including molecules comprising, or alternatively consisting of, antibody fragments or variants thereof), comprising, or alternatively consisting of, a VH domain having an amino acid sequence of any one of the VH domains expressed by at least one of the cell lines referred to in Table 1 or a VL domain having an amino acid sequence of any one of the VL domains expressed by at least one of the cell lines referred to in Table 1.

The present invention also provides antibodies that comprise, or alternatively consist of, variants (including derivatives) of the antibody molecules (e.g., the VH domains and/or VL domains) described herein, which antibodies immunospecifically bind to a TRAIL receptor or fragment or variant thereof. Standard techniques known to those of skill in the art can be used to introduce mutations in the nucleotide sequence encoding a molecule of the invention, including, for example, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VB domain, VHCDR1, VHCDR2, VHCDR3, VL domain, VLCDR1, VLCDR2, or VLCDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind a TRAIL receptor).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybriodma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein, (e.g., ability to immunospecifically bind a TRAIL receptor) can be determined using techniques described herein or by routinely modifying techniques known in the art.

In a specific embodiment, an antibody of the invention (including a molecule comprising, or alternatively consisting of, an antibody fragment or variant thereof), that immunospecifically binds TRAIL receptor polypeptides or fragments or variants thereof, comprises, or alternatively consists of, an amino acid sequence encoded by a nucleotide sequence that hybridizes to a nucleotide sequence that is complementary to that encoding one of the VH or VL domains expressed by one or more cell lines referred to in Table 1. under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3). Nucleic acid molecules encoding these antibodies are also encompassed by the invention.

It is well known within the art that polypeptides, or fragments or variants thereof, with similar amino acid sequences often have similar structure and many of the same biological activities. Thus, in one embodiment, an antibody (including a molecule comprising, or alternatively consisting of, an antibody fragment or variant thereof), that immunospecifically binds to a TRAIL receptor polypeptide or fragments or variants of a TRAIL receptor polypeptide, comprises, or alternatively consists of, a VH domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of a VH domain of a heavy chain expressed by at least one of the cell lines referred to in Table 1.

In another embodiment, an antibody (including a molecule comprising, or alternatively consisting of, an antibody fragment or variant thereof), that immunospecifically binds to a TRAIL receptor polypeptide or fragments or variants of a TRAIL receptor polypeptide, comprises, or alternatively consists of, a VL domain having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of a VL domain of a light chain expressed by at least one of the cell lines referred to in Table 1.

Methods of Producing Antibodies

Antibodies in accordance with the invention are preferably prepared the utilization of a transgenic mouse that has a substantial portion of the human antibody producing genome inserted but that is rendered deficient in the production of endogenous, murine, antibodies (e.g., XenoMouse strains available from Abgenix Inc., Fremont, Calif.). Such mice, then, are capable of producing human immunoglobulin molecules and antibodies and are deficient in the production of murine immunoglobulin molecules and antibodies. Technologies utilized for achieving the same are disclosed in the patents, applications, and references disclosed herein.

XenoMouse Technology

The ability to clone and reconstruct megabase-sized human loci in YACs and to introduce them into the mouse germline provides a powerful approach to elucidating the functional components of very large or crudely mapped loci as well as generating useful models of human disease. Furthermore, the utilization of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B cell development. Furthermore, such a strategy could provide an ideal source for production of fully human monoclonal antibodies (Mabs) an important milestone towards fulfilling the promise of antibody therapy in human disease.

Fully human antibodies are expected to minimize the immunogenic and allergic responses intrinsic to mouse or mouse-derivatized Monoclonal antibodies and thus to increase the efficacy and safety of the administered antibodies. The use of fully human antibodies can be expected to provide a substantial advantage in the treatment of chronic and recurring human diseases, such as cancer, which require repeated antibody administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human Monoclonal antibodies with the desired specificity could be readily produced and selected.

This general strategy was demonstrated in connection with the generation of the first XenoMouse™ strains as published in 1994. See Green et al. Nature Genetics 7:13-21 (1994). The XenoMouse™ strains were engineered with yeast artificial chromosomes (YACS) containing 245 kb and 10 190 kb-sized germline configuration fragments of the human heavy chain locus and kappa light chain locus, respectively, which contained core variable and constant region sequences. Id. The human Ig containing YACs proved to be compatible with the mouse system for both rearrangement and expression of antibodies and were capable of substituting for the inactivated mouse Ig genes. This was demonstrated by their ability to induce B-cell development, to produce an adult-like human repertoire of fully human antibodies, and to generate antigen-specific human monoclonal antibodies. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively, to produce XenoMouse™ mice. See Mendez et al. Nature Genetics 15:146-156 (1997), Green and Jakobovits J Exp. Med. 188:483-495 (1998), Green, Journal of Immunological Methods 231:11-23 (1999) and U.S. patent application Ser. No. 08/759,620, filed Dec. 3, 1996, the disclosures of which are hereby incorporated by reference.

Such approach is further discussed and delineated in U.S. patent application Ser. No. 07/466,008, filed Jan. 12, 1990, Ser. No. 07/710,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430, 938, Apr. 27, 1995, Ser. No. 08/464,584, filed Jun. 5, 1995, Ser. No. 08/464,582, filed Jun. 5, 1995, Ser. No. 08/471,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996. See also Mendez et al. Nature Genetics 15:146-156 (1997) and Green and Jakobovits J Exp. Med. 188:483 495 (1998). See also European Patent No., EP 0 471 151 B1, grant published Jun. 12, 1996, International Patent Application No., WO 94/02602, published Feb. 3, 1994, International Patent Application No., WO 96/34096, published Oct. 31, 1996, and WO 98/24893, published Jun. 11, 1998. The disclosures of each of the above-cited patents, applications, and references are hereby incorporated by reference in their entirety.

Human anti-mouse antibody (HAMA) responses have led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against TRAIL receptor polypeptides in order to vitiate concerns and/or effects of HAMA or HACA responses.

Monoclonal antibodies specific for TRAIL receptor polypeptides were prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 571-681 (1981)). Briefly, XenoMouse™ mice were immunized with TRAIL receptor polypeptides. After immunization, the splenocytes of such mice were extracted and fused with a suitable myeloma cell line. Any suitable myeloma cell line may be employed in accordance with the present invention; however, it is preferable to employ the parent myeloma cell line (SP2O), available from the ATCC. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the TRAIL receptor polypetides.

In one embodiment, the present invention provides hybridoma cell lines expressing an antibody of the invention. In specific embodiments, the hybridoma cell line of the invention is 1.2. In another specific embodiment, the hybridoma cell line of the invention is 1.2.1. In another specific embodiment, the hybridoma cell line of the invention is 1.2.2. In another specific embodiment, the hybridoma cell line of the invention is 1.2.3. In another specific embodiment, the hybridoma cell line of the invention is 1.3. In another specific embodiment, the hybridoma cell line of the invention is 1.3.1. In another specific embodiment, the hybridoma cell line of the invention is 1.3.2. In another specific embodiment, the hybridoma cell line of the invention is 1.3.3. In another specific embodiment, the hybridoma cell line of the invention is 7.1. In another specific embodiment, the hybridoma cell line of the invention is 7.1.1. In another specific embodiment, the hybridoma cell line of the invention is 7.1.2. In another specific embodiment, the hybridoma cell line of the invention is 7.1.3. In another specific embodiment, the hybridoma cell line of the invention is 7.3. In another specific embodiment, the hybridoma cell line of the invention is 7.3.1. In another specific embodiment, the hybridoma cell line of the invention is 7.3.2. In another specific embodiment, the hybridoma cell line of the invention is 7.3.3. In another specific embodiment, the hybridoma cell line of the invention is 7.8. In another specific embodiment, the hybridoma cell line of the invention is 7.8.1. In another specific embodiment, the hybridoma cell line of the invention is 7.8.2. In another specific embodiment, the hybridoma cell line of the invention is 7.8.3. In another specific embodiment, the hybridoma cell line of the invention is 7.10. In another specific embodiment, the hybridoma cell line of the invention is 7.10.1. In another specific embodiment, the hybridoma cell line of the invention is 7.10.2. In another specific embodiment, the hybridoma cell line of the invention is 7.10.3. In another specific embodiment, the hybridoma cell line of the invention is 7.12. In another specific embodiment, the hybridoma cell line of the invention is 7.12.1. In another specific embodiment, the hybridoma cell line of the invention is 7.12.2. In another specific embodiment, the hybridoma cell line of the invention is 7.12.3. In another specific embodiment, the hybridoma cell line of the invention is 8.3. In another specific embodiment, the hybridoma cell line of the invention is 8.3.1. In another specific embodiment, the hybridoma cell line of the invention is 8.3.2.

Additional Methods of Producing Antibodies

Antibodies of the invention (including antibody fragments or variants) can be produced by any method known in the art. For example, it will be appreciated that antibodies in accordance with the present invention can be expressed in cell lines including but not limited to myeloma cell lines and hybridoma cell lines. Sequences encoding the cDNAs or genomic clones for the particular antibodies can be used for transformation of a suitable mammalian or nonmammalian host cells or to generate phage display libraries, for example. Additionally, polypeptide antibodies of the invention may be chemically synthesized or produced through the use of recombinant expression systems.

One way to produce the antibodies of the invention would be to clone the VH and/or VL domains expressed by any one or more of the hybridoma cell lines referred to in Table 1. In order to isolate the VH and VL domains from the hybridoma cell lines, PCR primers including VH or VL nucleotide sequences (See Example 5), may be used to amplify the expressed VH and VL sequences contained in total RNA isolated from hybridoma cell lines. The PCR products may then be cloned using vectors, for example, which have a PCR product cloning site consisting of a 5′ and 3′ single T nucleotide overhang, that is complementary to the overhanging single adenine nucleotide added onto the 5′ and 3′ end of PCR products by many DNA polymerases used for PCR reactions. The VH and VL domains can then be sequenced using conventional methods known in the art.

The cloned VH and VL genes may be placed into one or more suitable expression vectors. By way of non-limiting example, PCR primers including VH or VL nucleotide sequences, a restriction site, and a flanking sequence to protect the restriction site may be used to amplify the VH or VL sequences. Utilizing cloning techniques known to those of skill in the art, the PCR amplified VH domains may be cloned into vectors expressing the appropriate immunoglobulin constant region, e.g., the human IgG1 or IgG4 constant region for VH domains, and the human kappa or lambda constant regions for kappa and lambda VL domains, respectively. Preferably, the vectors for expressing the VH or VL domains comprise a promoter suitable to direct expression of the heavy and light chains in the chosen expression system, a secretion signal, a cloning site for the immunoglobulin variable domain, immunoglobulin constant domains, and a selection marker such as neomycin. The VH and VL domains may also be cloned into a single vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art (See, for example, Guo et al., J. Clin. Endocrinol. Metab. 82:925-31 (1997), and Ames et al., J. Immunol. Methods 184:177-86 (1995) which are herein incorporated in their entireties by reference).

The invention provides polynucleotides comprising, or alternatively consisting of, a nucleotide sequence encoding an antibody of the invention (including molecules comprising, or alternatively consisting of, antibody fragments or variants thereof). The invention also encompasses polynucleotides that hybridize under high stringency, or alternatively, under intermediate or lower stringency hybridization conditions, e.g., as defined supra, to polynucleotides complementary to nucleic acids having a polynucleotide sequence that encodes an antibody of the invention or a fragment or variant thereof.

The polynucleotides may be obtained, and the nucleotide sequence of the polynucleotides determined, by any method known in the art. If the amino acid sequences of the VH domains, VL domains and CDRs thereof, are known, nucleotide sequences encoding these antibodies can be determined using methods well known in the art, i.e., the nucleotide codons known to encode the particular amino acids are assembled in such a way to generate a nucleic acid that encodes the antibody, of the invention. Such a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody (including molecules comprising, or alternatively consisting of, antibody fragments or variants thereof) may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells or Epstein Barr virus transformed B cell lines that express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody (including molecules comprising, or alternatively consisting of, antibody fragments or variants thereof) is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, VH and VL domains of heavy and light chains expressed by one or more cell lines referred to in Table 1, or fragments or variants thereof, are inserted within framework regions using recombinant DNA techniques known in the art. In a specific embodiment, one, two, three, four, five, six, or more of the CDRs of heavy and light chains expressed by one or more cell lines referred to in Table 1, or fragments or variants thereof, is inserted within framework regions using recombinant DNA techniques known in the art. The framework regions may be naturally occurring or consensus framework regions, and preferably human framework regions (see, e.g., Chothia et al., J. Mol. Biol. 278: 457-479 (1998) for a listing of human framework regions, the contents of which are hereby incorporated by reference in its entirety). Preferably, the polynucleotides generated by the combination of the framework regions and CDRs encode an antibody (including molecules comprising, or alternatively consisting of, antibody fragments or variants thereof) that specifically binds to a TRAIL receptor. Preferably, as discussed supra, polynucleotides encoding variants of antibodies or antibody fragments having one or more amino acid substitutions may be made within the framework regions, and, preferably, the amino acid substitutions do not significantly alter binding of the antibody to its antigen. Additionally, such methods may be used to make amino acid substitutions or deletions of one or more variable region cysteine residues participating in an intrachain disulfide bond to generate antibody molecules, or antibody fragments or variants, lacking one or more intrachain disulfide bonds. Other alterations to the polynucleotide are encompassed by the present invention and fall within the ordinary skill of the art.

For some uses, such as for in vitro affinity maturation of an antibody of the invention, it may be useful to express the VH and VL domains of the heavy and light chains expressed by one or more cell lines referred to in Table 1 as single schain antibodies or Fab fragments in a phage display library. For example, the cDNAs encoding the VH and VL domains expressed by the cell lines referred to in Table 1 may be expressed in all possible combinations using a phage display library, allowing for the selection of VH/VL combinations that bind a TRAIL receptor polypeptides with preferred binding characteristics such as improved affinity or improved off rates. Additionally, VH and VL segments—the CDR regions of the VH and VL domains expressed by the cell lines referred to in Table 1, in particular, may be mutated in vitro. Expression of VH and VL domains with “mutant” CDRs in a phage display library allows for the selection of VH/VL combinations that bind a TRAIL receptor polypeptides with preferred binding characteristics such as improved affinity or improved off rates.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues) or synthetic cDNA libraries. The DNA encoding the VH and VL domains are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an antigen of interest (i.e., a TRAIL receptor polypeptide or a fragment thereof) can be selected or identified with antigen, e.g., using labeled antigen or antigen bound or captured to a solid surface or bead. Examples of phage display methods that can be used to make the antibodies of the present invention include, but are not limited to, those disclosed in Brink-man et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280(1994); PCT application No. PCT/GB91/O1 134; PCT publications WO 90/02809; WO 91/10737; WO 92/01047; WO 92/18719; WO 93/1 1236; WO 95/15982; WO 95/20401; WO97/13844; and U.S. Pat. Nos. 5,698,426; 5,223,409; 5,403,484; 5,580,717; 5,427,908; 5,750,753; 5,821,047; 5,571,698; 5,427,908; 5,516,717; 5,780,225; 5,658,727; 5,735,743 and 5,969,108; each of which is incorporated herein by reference in its entirety.

For some uses, including in vivo use of antibodies in humans and in vitro detection assays, it may be preferable to use human or chimeric antibodies. Completely human antibodies are particularly desirable for therapeutic treatment of human patients. See also, U.S. Pat. Nos. 4,444,887 and 4,716,111; and PCT publications WO 98/46645, WO 98/50435, WO 98/24893, WO98/16654, WO 96/34096, WO 96/35735, and WO 91/10741; each of which is incorporated herein by reference in its entirety. In a specific embodiment, antibodies of the present invention comprise one or more VH and VL domains of the invention and constant regions from another immunoglobulin molecule, preferably a human immunoglobulin molecule. In a specific embodiment, antibodies of the present invention comprise one or more CDRs corresponding to the VH and VL domains of the invention and framework regions from another immunoglobulin molecule, preferably a human immunoglobulin molecule. In other embodiments, an antibody of the present invention comprises one, two, three, four, five, six or more VL CDRs or VH CDRs corresponding to one or more of the VH or VL domains expressed by one or more cell lines referred to in Table 1, or fragments or variants thereof, and framework regions (and, optionally one or more CDRs not present in the antibodies expressed by cell lines referred to in Table 1) from a human immunoglobulin molecule. In a preferred embodiment, an antibody of the present invention comprises a VH CDR3, VL CDR3, or both, corresponding to the same cell line, or different cell lines selected from the cell lines referred to in Table 1, or fragments or variants thereof, and framework regions from a human immunoglobulin.

A chimeric antibody is a molecule in which different portions of the antibody are derived from different immunoglobulin molecules such as antibodies having a human variable region and a non-human (e.g., murine) immunoglobulin constant region or vice versa. Methods for producing chimeric antibodies are known in the art. See e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entirety. Chimeric antibodies comprising one or more CDRs from human species and framework regions from a non-human immunoglobulin molecule (e.g., framework regions from a murine, canine or feline immunoglobulin molecule) (or vice versa) can be produced using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,352). In a preferred embodiment, chimeric antibodies comprise a human CDR3 having an amino acid sequence of any one of the VH CDR3s or VL CDR3s of a heavy or light chain expressed by one or more of the cell lines referred to in Table 1, or a variant thereof, and non-human framework regions or human framework regions different from those of the frameworks in the corresponding cell line disclosed in Table 1. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 352:323 (1988), which are incorporated herein by reference in their entireties.)

Intrabodies are antibodies, often scFvs, that are expressed from a recombinant nucleic aicd molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies of the invention can be produced using methods known in the art, such as those disclosed and reviewed in Chen et al., Hum. Gene Ther. 5:595-601 (1994); Marasco, W. A., Gene Ther. 4:11-15 (1997); Rondon and Marasco, Annu. Rev. Microbiol. 51:257-283 (1997); Proba et al., J. Mol. Biol. 275:245-253 (1998); Cohen et al., Oncogene 17:2445-2456 (1998); Ohage and Steipe, J. Mol. Biol. 291:1119-1128 (1999); Ohage et al., J. Mol. Biol. 291:1129-1134 (1999); Wirtz and Steipe, Protein Sci. 8:2245-2250 (1999); Zhu et al., J. Immunol. Methods 231:207-222 (1999); and references cited therein.

Recombinant expression of an antibody of the invention (including antibody fragments or variants thereof (e.g., a heavy or light chain of an antibody of the invention), requires construction of an expression vector(s) containing a polynucleotide that encodes the antibody. Once a polynucleotide encoding an antibody molecule (e.g., a whole antibody, a heavy or light chain of an antibody, or portion thereof (preferably, but not necessarily, containing the heavy or light chain variable domain)), of the invention has been obtained, the vector(s) for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention (e.g., a whole antibody, a heavy or light chain of an antibody, a heavy or light chain variable domain of an antibody, or a portion thereof or a heavy or light chain CDR, a single chain Fv, or fragments or variants thereof), operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464, the contents of each of which are hereby incorporated by reference in its entirety) and the variable domain of the antibody may be cloned into such a vector for expression of the entire heavy chain, the entire light chain, or both the entire heavy and light chains.

The expression vector(s) is(are) transferred to a host cell by conventional techniques and the transfected cells are then cultured by conventional techniques to produce an antibody of the invention. Thus, the invention includes host cells containing polynucleotide(s) encoding an antibody of the invention (e.g., whole antibody, a heavy or light chain thereof, or portion thereof, or a single chain antibody, or a fragment or variant thereof), operably linked to a heterologous promoter. In preferred embodiments, for the expression of entire antibody molecules, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include, but are not limited to, bacteriophage particles engineered to express antibody fragments or variants teherof (single chain antibodies), microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3, NS0 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990); Bebbington et al., Bio/Techniques 10:169 (1992); Keen and Hale, Cytotechnology 18:207 (1996)). These references are incorporated in their entirities by refernce herein.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., EMBO 1. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) may be used as a vector to express foreign genes. The virus grows in Spodoptera frugiperda cells. Antibody coding sequences may be cloned individually into non-essential regions (for example, the polyhedrin gene) of the virus and placed under control of an AcNPV promoter (for example, the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E-1 or E-3) will result in a recombinant virus that is viable and capable of expressing the antibody molecule in infected hosts (e.g., see Logan & Shenk, Proc. Natl. Acad. Sci. USA 8 1:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544(1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERY, BHK, Hela, COS, NSO, MDCK, 293, 3T3, W138, and in particular, breast cancer cell lines such as, for example, BT483, Hs578T, HTB2, BT2O and T47D, and normal mammary gland cell line such as, for example, CRL7O3O and HsS78Bst.

For long-term, high-yield production of recombinant proteins, stable expression is preferred. For example, cell lines which stably express the antibody may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., Cell 11:223 (1977)), hypoxanthineguanine phosphoribosyltransferase (Szybalska & Szybalski, Proc. Natl. Acad. Sci. USA 48:202 (1992)), and adenine phosphoribosyltransferase (Lowy et al., Cell 22:8 17 (1980)) genes can be employed in tk-, hgprt- or aprt- cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62: 191-217 (1993); TIB TECH 11(5):155-2 15 (May, 1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley. & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, “The use of vectors based on gene amplification for the expression of cloned genes in mammalian cells” in DNA Cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the coding sequence of the antibody, production of the antibody will also increase (Crouse et al., Mol. Cell. Biol. 3:257 (1983)).

Vectors which use glutamine synthase (GS) or DHFR as the selectable markers can be amplified in the presence of the drugs methionine sulphoximine or methotrexate, respectively. An advantage of glutamine synthase based vectors are the availabilty of cell lines (e.g., the murine myeloma cell line, NS0) which are glutamine synthase negative. Glutamine synthase expression systems can also function in glutamine synthase expressing cells (e.g. Chinese Hamster Ovary (CHO) cells) by providing additional inhibitor to prevent the functioning of the endogenous gene. A glutamine synthase expression system and components thereof are detailed in PCT publications: WO87/04462; WO86/05807; WO89/01036; WO89/10404; and WO91/06657 which are incorporated in their entireties by reference herein. Additionally, glutamine synthase expression vectors that may be used according to the present invention are commercially available from suplliers, including, for example Lonza Biologics, Inc. (Portsmouth, N.H.). Expression and production of monoclonal antibodies using a GS expression system in murine myeloma cells is described in Bebbington et al., Bio/technology 10:169(1992) and in Biblia and Robinson Biotechnol Prog. 11:1 (1995) which are incorporated in their entirities by reference herein.

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain is preferably placed before the heavy chain to avoid an excess of toxic free heavy chain (Proudfoot, Nature 322:52 (1986); Kohler, Proc. Natl. Acad. Sci. USA 77:2 197 (1980)). The coding sequences for the heavy and light chains may comprise cDNA or genomic DNA.

Once an antibody molecule of the invention (including molecules comprising, or alternatively consisting of, antibody fragments or variants thereof) has been chemically synthesized or recombinantly expressed, it may be purified by any method known in the art for purification of an immunoglobulin molecule, or more generally, a protein molecule, such as, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention may be fused to heterologous polypeptide sequences described herein or otherwise known in the art, to facilitate purification.

Antibodies of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the antibodies of the present invention may be glycosylated or may be non-glycosylated. In addition, antibodies of the invention may also include an initial modified methionine residue, in some cases as a result of host-mediated processes.

Antibodies of the invention can be chemically synthesized using techniques known in the art (e.g., see Creighton, 1983, Proteins: Structures and Molecular Principles, W.H. Freeman & Co., N.Y., and Hunkapiller, M., et al., 1984, Nature 310:105-111). For example, a peptide corresponding to a fragment of an antibody of the invention can be synthesized by use of a peptide synthesizer. Furthermore, if desired, nonclassical amino acids or chemical amino acid analogs can be introduced as a substitution or addition into the antibody polypeptide sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

The invention encompasses antibodies which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The antibodies may also be modified by association with a detectable label, such as an enzymatic, fluorescent, radioisotopic or affinity label to allow for detection and isolation of the antibody.

Examples of suitable enzymes include horseradish peroxidase, alkaline phosphatase, beta-galactosidase, glucose oxidase or acetylcholinesterase; examples of suitable prosthetic group complexes include streptavidin/biotin and avidin/biotin; examples of suitable fluorescent materials include biotin, umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include a radioactive metal ion, e.g., alpha-emitters such as, for example, ²¹³Bi, or other radioisotopes such as, for example, iodine (¹³¹I, ¹²⁵I, ¹²³I, ¹²¹I), carbon (¹⁴C), sulfur (³⁵S), tritium (³H), indium (^{115m}In, ^{113m}In, ¹¹²In, ¹¹¹In), and technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ₁₈₈Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Tin.

In specific embodiments, antibodies of the invention may be labeled with Europium. For example, antibodies of the invention may be labelled with Europium using the DELFIA Eu-labeling kit (catalog# 1244-302, Perkin Elmer Life Sciences, Boston, Mass.) following manufacturer's instructions.

In specific embodiments, antibodies of the invention are attached to macrocyclic chelators useful for conjugating radiometal ions, including but not limited to, ¹¹¹In, ¹⁷⁷Lu, ⁹⁰Y, ¹⁶⁶Ho, ¹⁵³Sm, ²¹⁵Bi and ²²⁵Ac to polypeptides. In a preferred embodiment, the radiometal ion associated with the macrocyclic chelators attached to antibodies of the invention is ¹¹¹In. In another preferred embodiment, the radiometal ion associated with the macrocyclic chelator attached to antibodies of the invention is ⁹⁰Y. In specific embodiments, the macrocyclic chelator is 1,4,7,10-tetraazacyclododecane-N,N′,N″,N′″-tetraacetic acid (DOTA). In specific embodiments, the macrocyclic chelator is α-(5-isothiocyanato-2-methoxyphenyl)-1,4,7,10-tetraaza-cyclododecane-1,4,7,10-tetraacetic acid. In other specific embodiments, the DOTA is attached to the antibody of the invention via a linker molecule. Examples of linker molecules useful for conjugating a macrocyclic chelator such as DOTA to a polypeptide are commonly known in the art—see, for example, DeNardo et al., Clin Cancer Res. 4(10):2483-90, 1998; Peterson et al., Bioconjug. Chem. 10(4):553-7, 1999; and Zimmerman et al., Nucl. Med. Biol. 26(8):943-50, 1999 which are hereby incorporated by reference in their entirety. In addition, U.S. Pat. Nos. 5,652,361 and 5,756,065, which disclose chelating agents that may be conjugated to antibodies, and methods for making and using them, are hereby incorporated by reference in their entireties.

Additional chelating agents, are known in the art. Chelator agents may be attached to antibodies of the invention to facilitate labeling said antibodies with metal ions including, but not limited to radionuclides or fluorescent labels. For example, see Subramanian, R. and Meares, C. F., “Bifunctional Chelating Agents for Radiometal-labeled monoclonal Antibodies,” in Cancer Imaging with Radiolabeled Antibodies (D. M. Goldenberg, Ed.) Kluwer Academic Publications, Boston; Saji, H., “Targeted delivery of radiolabeled imaging and therapeutic agents: bifunctional radiopharmaceuticals.” Crit. Rev. Ther. Drug Carrier Syst. 16:209-244 (1999); Srivastava S. C. and Mease R. C., “Progress in research on ligands, nuclides and techniques for labeling monoclonal antibodies.” Int. J. Rad. Appl. Instrum. B 18:589-603 (1991); and Liu, S. and Edwards, D. S., “Bifunctional chelators for therapeutic lanthanide radiopharmaceuticals.” Bioconjug. Chem. 12:7-34 (2001). Any chelator which can be covalently bound to an antibody may be used according to the present invention. The chelator may further comprise a linker moiety that connects the chelating moiety to the antibody.

In one embodiment, antibodies of the invention are attached to an acyclic chelator such as diethylene triamine-N,N,N′,N″,N″-pentaacetic acid (DPTA), analogues of DPTA, and/or derivatives of DPTA. As non-limiting examples, the chelator may be 2-(p-isothiocyanatobenzyl)-6-methyldiethylenetriaminepentaacetic acid (1B4M-DPTA, also known as MX-DTPA), 2-methyl-6-(rho-nitrobenzyl)-1,4,7-triazaheptane-N,N,N′,N″,N″-pentaacetic acid (nitro-1B4M-DTPA or nitro-MX-DTPA); 2-(p-isothiocyanatobenzyl)-cyclohexyldiethylenetriaminepentaacetic acid (CHX-DTPA), or N-[2-amino-3-(rho-nitrophenyl)propyl]-trans-cyclohexane-1,2-diamine-N,N′,N″-pentaacetic acid (nitro-CHX-A-DTPA).

In another embodiment, antibodies of the invention are attached to an acyclic terpyridine chelator such as 6,6″-bis[[N,N,N″,N″-tetra(carboxymethyl)amino]methyl]4′-(3-amino-4-methoxyphenyl)-2,2′:6′,2″-terpyridine (TMT-amine).

In specific embodiments, the macrocyclic chelator which is attached to the antibody of the invention is 1,4,7,10-tetraazacyclododecane-N,N′,N″,N′″-tetraacetic acid (DOTA). In other specific embodiments, the DOTA is attached to an antibody of the invention via a linker molecule. Examples of linker molecules useful for conjugating DOTA to a polypeptide are commonly known in the art—see, for example, DeNardo et al., Clin. Cancer Res. 4(10):2483-90, 1998; Peterson et al., Bioconjug Chem. 10(4):553-7, 1999; and Zimmerman et al., Nucl. Med. Biol. 26(8):943-50, 1999 which are hereby incorporated by reference in their entirety. In addition, U.S. Pat. Nos. 5,652,361 and 5,756,065, which disclose chelating agents that may be conjugated to antibodies, and methods for making and using them, are hereby incorporated by reference in their entireties. Though U.S. Pat. Nos. 5,652,361 and 5,756,065 focus on conjugating chelating agents to antibodies, one skilled in the art could readily adapt the method disclosed therein in order to conjugate chelating agents to other polypeptides.

Bifunctional chelators based on macrocyclic ligands in which conjugation is via an activated arm, or functional group, attached to the carbon backbone of the ligand can be employed using techniques described in the art, such as those described by M. Moi et al., J. Amer. Chem. Soc. 49:2639 (1989) (2-p-nitrobenzyl-1,4,7,10-tetraazacyclododecane-N,N′,N″,N′″-tetraacetic acid); S. V. Deshpande et al., J. Nucl. Med. 31:473 (1990); G. Ruser et al., Bioconj. Chem. 1:345 (1990); C. J. Broan et al., J. C. S. Chem. Comm. 23:1739 (1990); and C. J. Anderson et al., J. Nucl. Med. 36:850 (1995).

In one embodiment, a macrocyclic chelator, such as polyazamacrocyclic chelators, optionally containing one or more carboxy, amino, hydroxamate, phosphonate, or phosphate groups, are attached to antibodies of the invention. In another embodiment, the chelator is a chelator selected from the group consisting of DOTA, analogues of DOTA, and derivatives of DOTA.

In one embodiment, a suitable chelator molecule that may be attached to the antibodies of the invention include a chelator selected from the group: DOXA (1-oxa-4,7,10-triazacyclododecanetriacetic acid), NOTA (1,4,7-triazacyclononanetriacetic acid), TETA (1,4,8,11-tetraazacyclotetradecanetetraacetic acid), and THT (4′-(3-amino-4-methoxy-phenyl)-6,6″-bis(N′,N′-dicarboxymethyl-N-methylhydra zino)-2,2′:6′,2″-terpyridine), and analogs and derivatives thereof. See, e.g., Ohmono et al., J. Med. Chem. 35: 157-162 (1992); Kung et al., J. Nucl. Med. 25: 326-332 (1984); Jurisson et al., Chem. Rev. 93:1137-1156 (1993); and U.S. Pat. No. 5,367,080. Other suitable chelators include chelating agents disclosed in U.S. Pat. Nos. 4,647,447; 4,687,659; 4,885,363; EP-A-71564; WO89/00557; and EP-A-232751.

In another embodiment, suitable macrocyclic carboxylic acid chelators which can be used in the present invention include a chelator selected from the group: 1,4,7,10-tetraazacyclododecane-N,N′,N′″-tetraacetic acid (DOTA); 1,4,8,12-tetraazacyclopentadecane-N,N′,N″,N″-tetraacetic acid (15N4); 1,4,7-triazacyclononane-N,N′,N″-triacetic acid (9N3); 1,5,9-triazacyclododecane-N,N′,N″-triacetic acid (12N3); and 6-bromoacetamido-benzyl-1,4,8,1-tetraazacyclotetradecane-N,N′,N″,N″-tetraacetic acid (BAT).

A preferred chelator that can be attached to the antibodies of the invention is α-(5-isothiocyanato-2-methoxyphenyl)-1,4,7,10-tetraazacyclododecane-1,4,7,10-tetraacetic acid, which is also known as MeO-DOTA-NCS. A salt or ester of α-(5-isothiocyanato-2-methoxyphenyl)-1,4,7,10-tetraazacyclododecane-1,4,7,10-tetraacetic acid may also be used.

Antibodies of the invention to which chelators such as those described are covalently attached may be labeled (via the coordination site of the chelator) with radionuclides that are suitable for therapeutic, diagnostic, or both therapeutic and diagnostic purposes. Examples of appropriate metals include, but are not limited to, Ag, At, Au, Bi, Cu, Ga, Ho, In, Lu, Pb, Pd, Pm, Pr, Rb, Re, Rh, Sc, Sr, Tc, Tl, Y, and Yb. Examples of the radionuclide used for diagnostic purposes include, but are not limited to, Fe, Gd, ¹¹In, ⁶⁷Ga, or ⁶⁸Ga. In another embodiment, the radionuclide used for diagnostic purposes is ¹¹¹In or ⁶⁷Ga. Examples of the radionuclide used for therapeutic purposes include, but are not limited to, ¹⁶⁶Ho, ¹⁶⁵Dy, ⁹⁰Y, ¹¹⁵In, ⁵²Fe, or ⁷²Ga. In one embodiment, the radionuclide used for diagnostic purposes is ¹⁶⁶Ho or ⁹⁰Y. Examples of the radionuclides used for both therapeutic and diagnostic purposes include, but are not limited to, ¹⁵³Sm, ¹⁷⁷Lu, ¹⁵⁹Gd, ¹⁷⁵Yb, or ⁴⁷Sc. In one embodiment, the radionuclide is ¹⁵³Sm, ¹⁷⁷Lu, ¹⁷⁵Yb, or ¹⁵⁹Gd.

Preferred metal radionuclides that may be used according to the present invention include a radionuclide selected from ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁷⁷Lu, ⁹⁹Au, ⁴⁷Sc, ⁶⁷Ga, ⁵¹Cr, ^{177m}Sn, ⁶⁷Cu, ¹⁶⁷Tm, ⁹⁵Ru, ¹⁸⁸Re, ¹⁷⁷Lu, ¹⁹⁹Au, ²⁰³Pb and ¹⁴¹Ce.

In a particular embodiment, antibodies of the invention to which chelators are covalently attached may be labeled with a metal ion selected from the group consisting of ⁹⁰Y, ¹¹¹In, ¹⁷⁷Lu, ¹⁶⁶Ho, ²¹⁵Bi, and ²²⁵Ac.

Moreover, γ-emitting radionuclides, such as ^{99m}Tc, ¹¹¹In, ⁶⁷Ga, and ¹⁶⁹Yb have may be used for diagnostic imaging, while β-emitters, such as ⁶⁷Cu, ¹¹¹Ag, ¹⁸⁶Re, and ⁹⁰Y are useful for the applications in tumor therapy. Also other useful radionuclides include γ-emitters, such as ^{99m}Tc, ¹¹¹In, ⁶⁷Ga, and ¹⁶⁹Yb, and β-emitters, such as ⁶⁷Cu, ¹¹¹Ag, ¹⁸⁶Re, ¹⁸⁸Re and ⁹⁰Y, as well as other radionuclides of interest such as ²¹¹At, ²¹²Bi, ¹⁷⁷Lu, ⁸⁶Rb, ¹⁰⁵Rh, ¹⁵³Sm, ¹⁹⁸Au, ¹⁴⁹Pm, ⁸⁵Sr, ¹⁴²Pr, ²¹⁴Pb, ¹⁰⁹Pd, ¹⁶⁶Ho, ²⁰⁸Tl, and ⁴⁴Sc. Antibodies of the invention to which chelators are covalently attached may be labeled with the radionuclides described above or others known in the art.

In another embodiment, antibodies of the invention to which chelators are covalently attached may be labeled with paramagnetic metal ions including ions of transition and lanthanide metal, such as metals having atomic numbers of 21-29, 42, 43, 44, or 57-71, in particular ions of metals selected from Cr, V, Mn, Fe, Co, Ni, Cu, La, Ce, Pr, Nd, Pm, Sm, Eu, Gd, Th, Dy, Ho, Er, Tm, Yb, and Lu. The paramagnetic metals used in compositions for magnetic resonance imaging include the elements having atomic numbers of 22 to 29, 42, 44 and 58-70.

In another embodiment, antibodies of the invention to which chelators are covalently attached may be labeled with fluorescent metal ions including lanthanides, in particular a member selected from La, Ce, Pr, Nd, Pm, Sm, Eu (e.g., ¹⁵²Eu), Gd, Th, Dy, Ho, Er, Tm, Yb, and Lu.

In another embodiment, antibodies of the invention to which chelators are covalently attached may be labeled with heavy metal-containing reporters including atoms of a metal selcted from Mo, Bi, Si, and W.

Radiolabeled antibodies of the invention may be used not only to kill cells to which they bind, but also may be useful to kill neighboring cells. For example, expression of TR4 may not be universal on all the cells of the tumor. However, because the energy from certain radioactive decay events can span more than a single cell diameter, radiolabeled antibodies of the invention may be used to kill cells that do not express TR4, e.g., cancerous cells, but which are in close proximity to cells that do express TR4.

In one embodiment, antibodies of the invention are labeled with biotin. In other related embodiments, biotinylated antibodies of the invention may be used, for example, as an imaging agent or as a means of identifying one or more TRAIL Receptor coreceptor or ligand molecule.

Also provided by the invention are chemically modified derivatives of antibodies of the invention which may provide additional advantages such as increased solubility, stability and in vivo or in vitro circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The antibodies may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000,30,000,35,000, 40,000, 50,000, 55,000, 60,000,65,000, 70,000, 75,000, 80,000, 85,000,90,000,95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

The polyethylene glycol molecules (or other chemical moieties) should be attached to the antibody with consideration of effects on functional or antigenic domains of the antibody. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include, for example, lysine residues and the N-terminal amino acid residues; those having a free carboxyl group may include aspartic acid residues, glutamic acid residues, and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins, e.g., antibodies, via linkage to any of a number of amino acid residues. For example, polyethylene glycol can be linked to a proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

One may specifically desire antibodies chemically modified at the N-terminus of either the heavy chain or the light chain or both. Using polyethylene glycol as an illustration, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective chemical modification at the N-terminus may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

As indicated above, pegylation of the antibodies of the invention may be accomplished by any number of means. For example, polyethylene glycol may be attached to the antibody either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466, the disclosures of each of which are incorporated herein by reference.

One system for attaching polyethylene glycol directly to amino acid residues of antibodies without an intervening linker employs tresylated MPEG, which is produced by the modification of monmethoxy polyethylene glycol (MPEG) using tresylchloride (CISO2CH2CF3). Upon reaction of protein with tresylated MPEG, polyethylene glycol is directly attached to amine groups of the protein. Thus, the invention includes antibody-polyethylene glycol conjugates produced by reacting antibodies of the invention with a polyethylene glycol molecule having a 2,2,2-trifluoreothane sulphonyl group.

Polyethylene glycol can also be attached to antibodies using a number of different intervening linkers. For example, U.S. Pat. No. 5,612,460, the entire disclosure of which is incorporated herein by reference, discloses urethane linkers for connecting polyethylene glycol to proteins. Antibody-polyethylene glycol conjugates wherein the polyethylene glycol is attached to the antibody by a linker can also be produced by reaction of antibodies with compounds such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number additional polyethylene glycol derivatives and reaction chemistries for attaching polyethylene glycol to proteins are described in WO 98/32466, the entire disclosure of which is incorporated herein by reference. Pegylated antibody products produced using the reaction chemistries set out herein are included within the scope of the invention.

The number of polyethylene glycol moieties attached to each antibody of the invention (i.e., the degree of substitution) may also vary. For example, the pegylated antibodies of the invention may be linked, on average, to 1, 2, 3, 4, 5, 6, 7, 8, 9, 10, 12, 15, 17, 20, or more polyethylene glycol molecules. Similarly, the average degree of substitution within ranges such as 1-3, 24, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per antibody molecule. Methods for determining the degree of substitution are discussed, for example, in Delgado et al., Crit. Rev. Thera. Drug Carrier Sys. 9:249-304 (1992).

Characterization of Anti-TRAIL Receptor Antibodies

Antibodies of the present invention (including molecules comprising, or alternatively consisting of, antibody fragments or variants thereof) may also be described or specified in terms of their binding to TRAIL receptor polypeptides or fragments or variants of TRAIL receptor polypeptides. In specific embodiments, antibodies of the invention bind TRAIL receptor polypeptides, or fragments or variants thereof, with a dissociation constant or K_{D} of less than or equal to 5×10² M, 10⁻² M, 5×10⁻³ M, 10⁻³ M, 5×10⁻⁴ M, 10⁻⁴ M, 5×10⁻⁵ M, or 10⁻⁵ M. More preferably, antibodies of the invention bind TRAIL receptor polypeptides or fragments or variants thereof with a dissociation constant or K_{D} less than or equal to 5×10⁻⁶ M, 10⁻⁶ M, 5×10⁻⁷ M, 10⁻⁷ M, 5×10⁻⁸ M, or 10⁻⁸ M. Even more preferably, antibodies of the invention bind TRAIL receptor polypeptides or fragments or variants thereof with a dissociation constant or K_{D} less than or equal to 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M 5×10⁻¹⁴ M, 10⁻¹⁴ M, 5×10⁻¹⁵ M, or 10⁻¹⁵ M. The invention encompasses antibodies that bind TRAIL Receptor polypeptides with a dissociation constant or K_{D} that is within one of the ranges that are between each of the individual recited values.

In specific embodiments, antibodies of the invention bind TRAIL receptor polypeptides or fragments or variants thereof with an off rate (k_{off}) of less than or equal to 5×10⁻² sec⁻, 10⁻² sec⁻¹, 5×10⁻³ sec⁻¹ or 10⁻³ sec⁻¹. More preferably, antibodies of the invention bind TRAIL receptor polypeptides or fragments or variants thereof with an off rate (k_{off}) less than or equal to 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹. The invention encompasses antibodies that bind TRAIL Receptor polypeptides with an off rate (k_{off}) that is within one of the ranges that are between each of the individual recited values.

In other embodiments, antibodies of the invention bind TRAIL receptor polypeptides or fragments or variants thereof with an on rate (kₒₙ) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, antibodies of the invention bind TRAIL receptor polypeptides or fragments or variants thereof with an on rate (ken) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹. The invention encompasses antibodies that bind TRAIL Receptor polypeptides with on rate (kₒₙ) that is within one of the ranges that are between each of the individual recited values.

The antibodies of the invention (including molecules comprising, or alternatively consisting of, antibody fragments or variants thereof) immunospecifically bind to a polypeptide or polypeptide fragment or variant of human TRAIL receptor polypeptides (SEQ ID NOS:1-4). In another embodiment, the antibodies of the invention immunospecifically bind to a polypeptide or polypeptide fragment or variant of simian TRAIL receptor polypeptides. In yet another embodiment, the antibodies of the invention immunospecifically bind to a polypeptide or polypeptide fragment or variant of murine TRAIL receptor polypeptides. In one embodiment, the antibodies of the invention bind immunospecifically to human and simian TRAIL receptor polypeptides. In another embodiment, the antibodies of the invention bind immunospecifically to human TRAIL receptor polypeptides and murine TRAIL receptor polypeptides. More preferably, antibodies of the invention, preferentially bind to human TRAIL receptor polypeptides compared to murine TRAIL receptor polypeptides.

In preferred embodiments, the antibodies of the present invention (including molecules comprising, or alternatively consisting of, antibody fragments or variants thereof), immunospecifically bind to TRAIL receptor polypeptides and do not cross-react with any other antigens. In preferred embodiments, the antibodies of the invention immunospecifically bind to TRAIL receptor polypeptides (e.g., SEQ ID NOS:1-4 or fragments or variants thereof) and do not cross-react with one or more additional members of the Tumor Necrosis Factor Tumor Necrosis Factor Receptor Family polypeptides (e.g., BCMA, TAC1, CD30, CD27, OX40, 4-1BB, CD40, NGFR, TNFR1, TNFR2, Fas, and NGFR).

In another embodiment, the antibodies of the present invention (including molecules comprising, or alternatively consisting of, antibody fragments or variants thereof), immunospecifically bind to TRAIL receptor polypeptides and cross-react with other antigens. In other embodiments, the antibodies of the invention immunospecifically bind to TRAIL receptor polypeptides (e.g., SEQ ID NOS:1-4 or fragments or variants thereof) and cross-react with one or more additional members of the Tumor Necrosis Factor Receptor Family polypeptides (e.g., BCMA, TAC1, CD30, CD27, OX40, 4-1BB, CD40, NGFR, TNFR1, TNFR2, Fas, and NGFR).

In a preferred embodiment, antibodies of the invention preferentially bind TR4 (SEQ ID NO:1), or fragments and variants thereof relative to their ability to bind TR5, TR7, or TR10 (SEQ ID NOS:2-4) or fragments or variants thereof. In another preferred embodiment, antibodies of the invention preferentially bind TR7, or fragments and variants thereof relative to their ability to bind TR4, TR5, or TR10 (SEQ ID NOS:1, 2, and 4) or fragments or variants thereof. In other preferred embodiments, the antibodies of the invention preferentially bind to TR4 and TR7 (SEQ ID NOS:1 and 3), or fragments and variants thereof relative to their ability to bind TR5 or TR10 (SEQ ID NOS:2 and 4) or fragments or variants thereof. In other preferred embodiments, the antibodies of the invention preferentially bind to TR5 and TR10 (SEQ ID NOS:2 and 4), or fragments and variants thereof relative to their ability to bind TR4 or TR7 (SEQ ID NOS:1 and 3) or fragments or variants thereof. In other preferred embodiments, the antibodies of the invention bind TR4, TR5, TR7 and TR10 (SEQ ID NOS:1-4). In another embodiment, antibodies of the invention preferentially bind TR5 (SEQ ID NO:2), or fragments and variants thereof relative to their ability to bind TR4, TR7 or TR10 (SEQ ID NOS:1, 4 and 5) or fragments or variants thereof. In another embodiment, antibodies of the invention preferentially bind TR10 (SEQ ID NO:4), or fragments and variants thereof relative to their ability to bind TR4, TR5, or TR7 (SEQ ID NOS:1-3) or fragments or variants thereof. An antibody's ability to preferentially bind one antigen compared to another antigen may be determined using any method known in the art.

By way of non-limiting example, an antibody may be considered to bind a first antigen preferentially if it binds said first antigen with a dissociation constant (K_{D}) that is less than the antibody's K_{D} for the second antigen. In another non-limiting embodiment, an antibody may be considered to bind a first antigen preferentially if it binds said first antigen with an affinity (K_{D}) that is at least one order of magnitude less than the antibody's K_{D} for the second antigen. In another non-limiting embodiment, an antibody may be considered to bind a first antigen preferentially if it binds said first antigen with an affinity (K_{D}) that is at least two orders of magnitude less than the antibody's K_{D} for the second antigen.

In another non-limiting embodiment, an antibody may be considered to bind a first antigen preferentially if it binds said first antigen with an off rate (k_{off}) that is less than the antibody's k_{off} for the second antigen. In another non-limiting embodiment, an antibody may be considered to bind a first antigen preferentially if it binds said first antigen with a k_{off} that is at least one order of magnitude less than the antibody's k_{off} for the second antigen. In another non-limiting embodiment, an antibody may be considered to bind a first antigen preferentially if it binds said first antigen with a k_{off} that is at least two orders of magnitude less than the antibody's k_{off} for the second antigen.

The invention also encompasses antibodies (including molecules comprising, or alternatively consisting of, antibody fragments or variants thereof) that have one or more of the same biological characteristics as one or more of the antibodies described herein. By “biological characteristics” is meant, the in vitro or in vivo activities or properties of the antibodies, such as, for example, the ability to bind to TRAIL receptor polypeptides (e.g., membrane-embedded TRAIL receptors), the ability to stimulate TRAIL receptor mediated biological activity (e.g., to stimulate apoptosis of TRAIL receptor expressing cells, see Example 4); the ability to substantially block TRAIL receptor ligand (e.g. TRAIL (SEQ ID NO:5), also known as AIM-I, International Application No. WO 97/35899 and U.S. Pat. No. 5,771,223), or a fragment, variant or fusion protein thereof, binding to TRAIL receptor, see Example 3; or the ability to upregulate TRAIL receptor expression on the surface of cells. Other biological activities that antibodies against TRAIL receptor polypeptides may have, include, but are not limited to, the ability to inhibit TRAIL receptor mediated biological activity (e.g., to inhibit apoptosis of TRAIL receptor expressing cells) or the ability to downregulate TRAIL receptor expression on the surface of cells. Optionally, the antibodies of the invention will bind to the same or closely associated (e.g., overlapping) epitope as at least one of the antibodies specifically referred to herein. Such epitope binding can be routinely determined using assays known in the art.

The present invention also provides for antibodies (including molecules comprising, or alternatively consisting of, antibody fragments or variants thereof), that stimulate one or more TRAIL receptor polypeptide mediated biological activities. In one embodiment, an antibody that stimulates one or more TRAIL receptor polypeptide mediated biological activities comprises, or alternatively consists of a VH and/or a VL domain of a heavy chain and/or a light chain, respectively, expressed by at least one of the cell lines referred to in Table 1, or fragment or variant thereof. In a specific embodiment, an antibody that stimulates one or more TRAIL receptor polypeptide mediated biological activities comprises, or alternatively consists of a VH and a VL domain of a heavy chain and a light chain, respectively, expressed by any one of the cell lines referred to in Table 1, or fragment or variant thereof. Nucleic acid molecules encoding these antibodies are also encompassed by the invention.

The present invention also provides for antibodies (including molecules comprising, or alternatively consisting of, antibody fragments or variants thereof), that stimulate apoptosis of TRAIL receptor expressing cells (see Example 4). In one embodiment, an antibody that stimulates apoptosis of TRAIL receptor expressing cells comprises, or alternatively consists of a VR and/or a VL domain of a heavy chain and/or a light chain, respectively, expressed by at least one of the cell lines referred to in Table 1, or fragment or variant thereof. In a specific embodiment, an antibody that stimulates apoptosis of TRAIL receptor expressing cells comprises, or alternatively consists of a VH and a VL domain of a heavy chain and a light chain, respectively, expressed by any one of the cell lines referred to in Table 1, or fragment or variant thereof. Nucleic acid molecules encoding these antibodies are also encompassed by the invention.

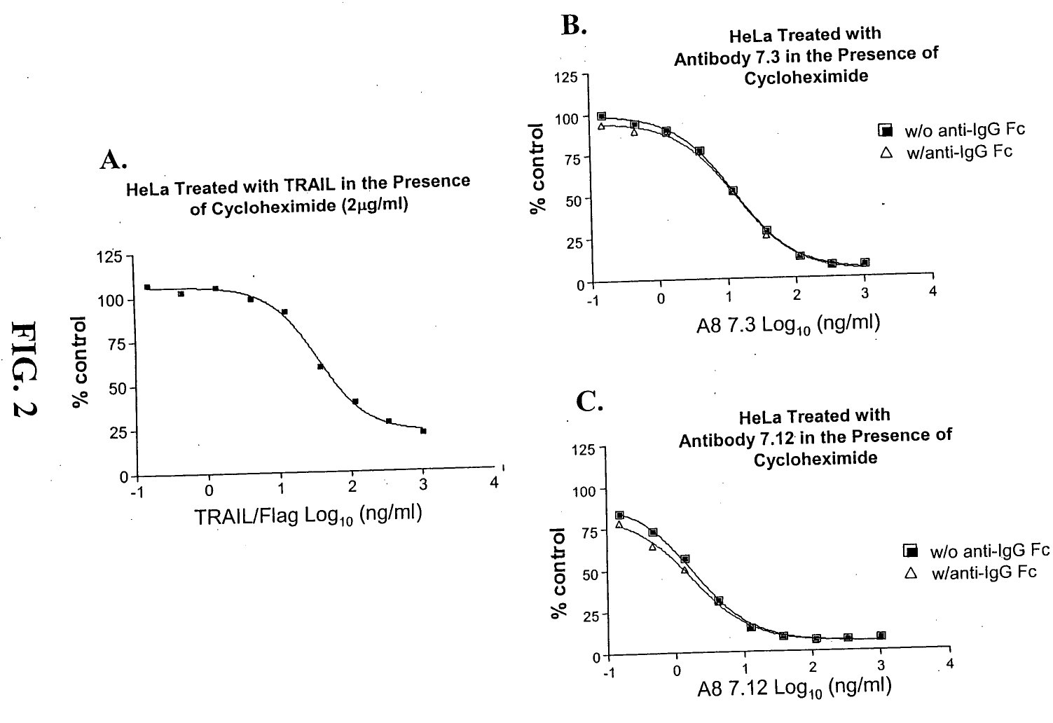

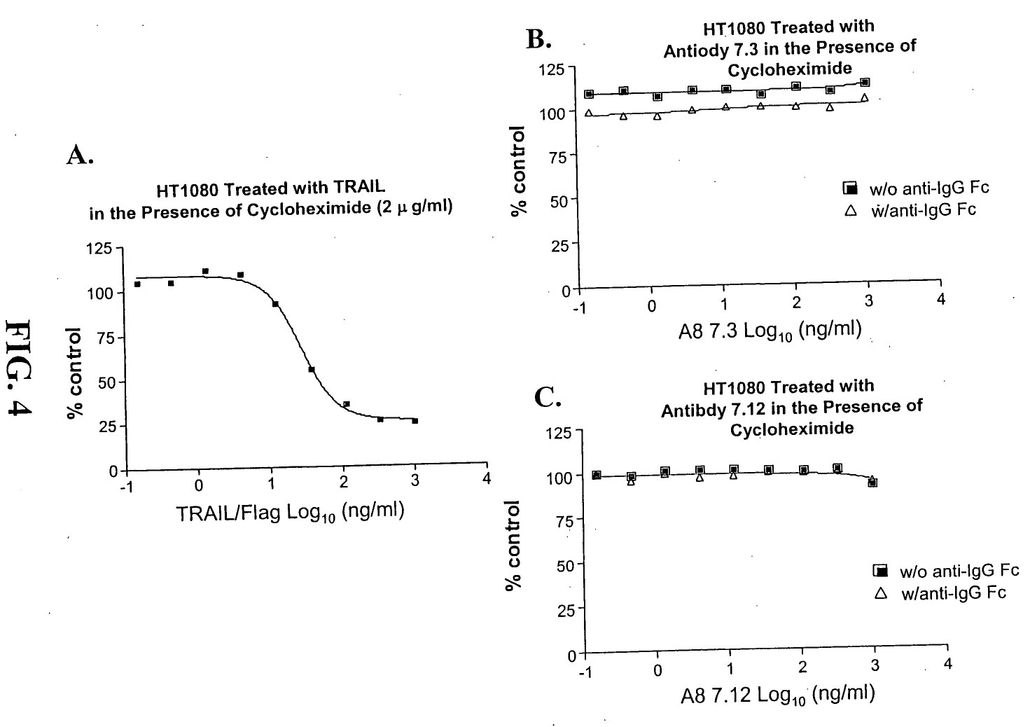

In preferred embodiments, the present invention also provides for antibodies (including molecules comprising, or alternatively consisting of, antibody fragments or variants thereof), that stimulate apoptosis of TRAIL receptor expressing cells equally well in the presence or absence of antibody cross-linking reagents, such as for example anti-Ig Fe reagents cells (See, for example, Example 4 and FIGS. 2, 3, and 4). In a specific embodiment, antibodies of the present invention stimulate apoptosis of HeLa cells, equally well in the presence or absence of an anti-Ig Fe antibody cross-linking reagent. In another specific embodiment, antibodies of the present invention stimulate apoptosis of HeLa cells, equally well in the presence or absence of an anti-Ig Fe antibody cross-linking reagent in the presence of 2 micrograms/milliliter of cycloheximide. In another embodiment, antibodies of the present invention stimulate apoptosis of SW480 cells, equally well in the presence or absence of an anti Ig Fe antibody cross-linking reagent. In another specific embodiment, antibodies of the present invention stimulate apoptosis of SW480 cells, equally well in the presence or absence of an anti-Ig Fe antibody cross-linking reagent in the presence of 2 micrograms/milliliter of cycloheximide.

In other preferred embodiments, the present invention also provides for antibodies (including molecules comprising, or alternatively consisting of, antibody fragments or variants thereof), that stimulate apoptosis of TRAIL receptor expressing cells at least as well as an equal concentration (in terms of, for example, nanograms/milliliter) of TRAIL polypeptide (including TRAIL polypeptide fragments, variants or fusion proteins) stimulates apoptosis of TRAIL receptor expressing cells (See, for example, Example 4 and FIGS. 2, 3, and 4). In a specific embodiment, antibodies of the invention stimulate apoptosis of TRAIL receptor expressing cells better than an equal concentration (in terms of, for example, nanograms/milliliter) of TRAIL polypeptide (including TRAIL polypeptide fragments, variants or fusion proteins) stimulates apoptosis of TRAIL receptor expressing cells. In a specific embodiment, antibodies of the invention stimulate apoptosis of HeLa cells better than an equal concentration (in terms of, for example, nanograms/milliliter) of TRAIL polypeptide (including TRAIL polypeptide fragments, variants or fusion proteins) stimulates apoptosis of TRAIL receptor expressing cells. In another specific embodiment, antibodies of the present invention stimulate apoptosis of HeLa cells better than an equal concentration (in terms of, for example, nanograms/milliliter) of TRAIL polypeptide (including TRAIL polypeptide fragments, variants or fusion proteins) stimulates apoptosis of TRAIL receptor expressing cells in the presence of 2 micrograms/milliliter of cycloheximide.

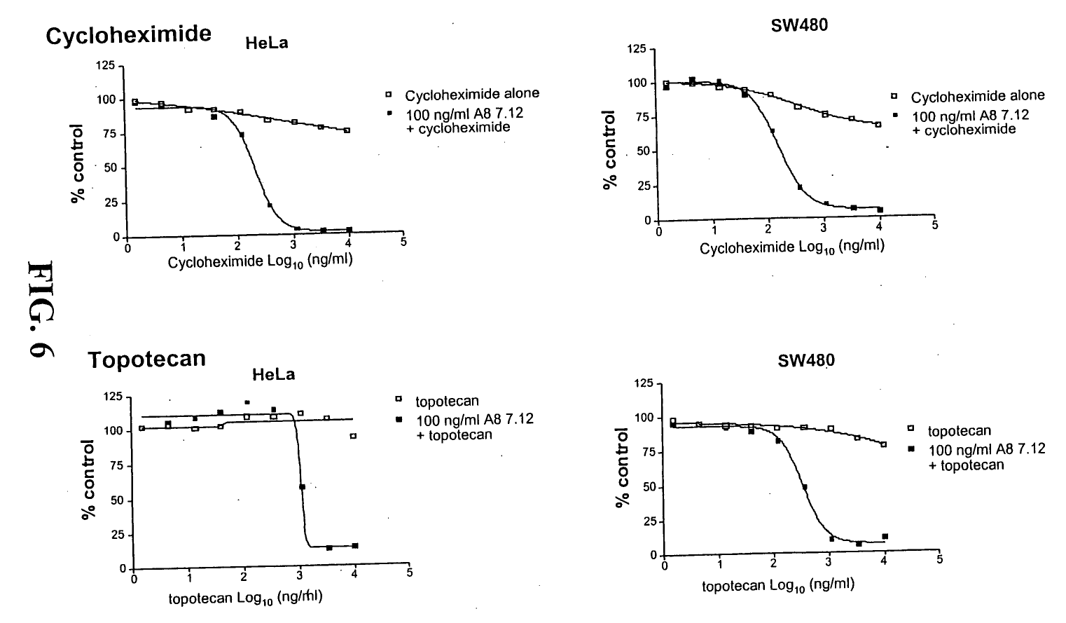

In other preferred embodiments, the present invention also provides for antibodies (including molecules comprising, or alternatively consisting of, antibody fragments or variants thereof), that stimulate more apoptosis of TRAIL receptor expressing cells when administered in combination with a chemotherapeutic drug (or other therapeutic agents useful in the treatment of cancers), than either the chemotherapeutic drug (or other therapeutic agents useful in the treatment of cancers) or the antibodies alone stimulate apoptosis of receptor expressing cells (see, for example, FIG. 6). In specific embodiments, antibodies of the present invention, stimulate more apoptosis of TRAIL receptor expressing cells when administered in combination with Topotecan, than either Topotecan or the antibodies alone stimulate apoptosis of receptor expressing cells. In specific embodiments, antibodies of the present invention, stimulate more apoptosis of TRAIL receptor expressing cells when administered in combination with cycloheximide, than either cycloheximide or the antibodies alone stimulate apoptosis of receptor expressing cells.

The present invention also provides for antibodies (including molecules comprising, or alternatively consisting of, antibody fragments or variants thereof), that blocks or inhibits the binding of TRAIL to a TRAIL receptor polypeptide (see Example 3). In one embodiment, an antibody that blocks or inhibits the binding of TRAIL to a TRAIL receptor polypeptide comprises, or alternatively consists of a VH and/or a VL domain of a heavy chain and/or a light chain, respectively, expressed by at least one of the cell lines referred to in Table 1, or fragment or variant thereof. In a specific embodiment, an antibody that blocks or inhibits the binding of TRAIL to a TRAIL receptor polypeptide comprises, or alternatively consists of a VH and a VL domain of a heavy chain and a light chain, respectively, expressed by any one of the cell lines referred to in Table 1, or fragment or variant thereof. Nucleic acid molecules encoding these antibodies are also encompassed by the invention.

Antibodies of the present invention (including antibody fragments or variants thereof) may be characterized in a variety of ways. In particular, antibodies and related molecules of the invention may be assayed for the ability to immunospecifically bind to TRAIL receptor polypeptides or a fragment or variant of TRAIL receptor polypeptides using techniques described herein or routinely modifying techniques known in the art. Assays for the ability of the antibodies of the invention to immunospecifically bind TRAIL receptor polypeptides or a fragment of TRAIL receptor polypeptides may be performed in solution (e.g., Houghten, Bio/Techniques 13:412-421(1992)), on beads (e.g., Lam, Nature 354:82-84 (1991)), on chips (e.g., Fodor, Nature 364:555-556 (1993)), on bacteria (e.g., U.S. Pat. No. 5,223,409), on spores (e.g., U.S. Pat. Nos. 5,571,698; 5,403,484; and 5,223,409), on plasmids (e.g., Cull et al., Proc. Natl. Acad. Sci. USA 89:1865-1869 (1992)) or on phage (e.g., Scott and Smith, Science 249:386-390 (1990); Devlin, Science 249:404-406 (1990); Cwirla et al., Proc. Natl. Acad. Sci. USA 87:7178-7182 (1990); and Felici, J. Mol. Biol. 222:301-310 (1991)) (each of these references is incorporated herein in its entirety by reference). Antibodies that have been identified to immunospecifically bind to TRAIL receptor polypeptides or a fragment or variant of TRAIL receptor polypeptides can then be assayed for their specificity and affinity for TRAIL receptor polypeptides or a fragment of TRAIL receptor polypeptides using or routinely modifying techniques described herein or otherwise known in the art.

The antibodies of the invention may be assayed for immunospecific binding to TRAIL receptor polypeptides and cross-reactivity with other antigens by any method known in the art. Immunoassays which can be used to analyze immunospecific binding and cross-reactivity include, but are not limited to, competitive and non-competitive assay systems using techniques such as BIAcore analysis (See, e.g., Example 2), FACS (fluorescence activated cell sorter) analysis (see, e.g., Example 4 and FIG. 1), immunofluorescence, immunocytochemistry, radioimmunoassays, ELISA (enzyme linked immunosorbent assay), “sandwich” immunoassays, immunoprecipitation assays, western blots, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

ELISAs comprise preparing antigen, coating the well of a 96-well microtiter plate with the antigen, washing away antigen that did not bind the wells, adding the antibody of interest conjugated to a detectable compound such as an enzymatic substrate (e.g., horseradish peroxidase or alkaline phosphatase) to the wells and incubating for a period of time, washing away unbound antibodies or non-specifically bound antibodies, and detecting the presence of the antibodies specifically bound to the antigen coating the well. In ELISAs, the antibody of interest does not have to be conjugated to a detectable compound; instead, a second antibody (which recognizes the antibody of interest) conjugated to a detectable compound may be added to the well. Alternatively, the antigen need not be directly coated to the well; instead the ELISA plates may be coated with an anti-Ig Fc antibody, and the antigen in the form or a TRAIL receptor-Fc fusion protein, may be bound to the anti-Ig Fe coated to the plate. This may be desirable so as to maintain the antigen protein (e.g., the TRAIL receptor polypeptides) in a more native conformation than it may have when it is directly coated to a plate. In another alternative, instead of coating the well with the antigen, the antibody may be coated to the well. In this case, the detectable molecule could be the antigen conjugated to a detectable compound such as an enzymatic substrate (e.g., horseradish peroxidase or alkaline phosphatase). One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1.

The binding affinity of an antibody (including an scFv or other molecule comprising, or alternatively consisting of, antibody fragments or variants thereof) to an antigen and the off-rate of an antibody-antigen interaction can be determined by competitive binding assays. One example of a competitive binding assay is a radioimmunoassay comprising the incubation of labeled antigen (e.g., antigen labeled with ³H or ¹²⁵I), or fragment or variant thereof with the antibody of interest in the presence of increasing amounts of unlabeled antigen, and the detection of the antibody bound to the labeled antigen. The affinity of the antibody of the present invention for TRAIL receptor and the binding off-rates can be determined from the data by Scatchard plot analysis. Competition with a second antibody can also be determined using radioimmunoassays. In this case, TRAIL receptor polypeptide is incubated with an antibody of the present invention conjugated to a labeled compound (e.g., compound labeled with ³H or ¹²⁵I) in the presence of increasing amounts of an unlabeled second anti-TRAIL receptor antibody. This kind of competitive assay between two antibodies, may also be used to determine if two antibodies bind the same, closely associated (e.g., overlapping) or different epitopes.

In a preferred embodiment, BIAcore kinetic analysis is used to determine the binding on and off rates of antibodies (including antibody fragments or variants thereof) to a TRAIL receptor, or fragments of a TRAIL receptor. BIAcore kinetic analysis comprises analyzing the binding and dissociation of antibodies from chips with immobilized TRAIL receptors on their surface as described in detail in Example 2.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1 to 4 hours) at 40 degrees C., adding protein A and/or protein G sepharose beads to the cell lysate, incubating for about an hour or more at 40 degrees C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, 1994, Current Protocols in Molecular Biology, Vol. 1, John Wiley & Sons, Inc., New York at 10.8.1.

Antibody Conjugates

The present invention encompasses antibodies (including antibody fragments or variants thereof), recombinantly fused or chemically conjugated (including both covalent and non-covalent conjugations) to a heterologous polypeptide (or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences. For example, antibodies of the invention may be used to target heterologous polypeptides to particular cell types (e.g., cancer cells), either in vitro or in vivo, by fusing or conjugating the heterologous polypeptides to antibodies of the invention that are specific for particular cell surface antigens or which bind antigens that bind particular cell surface receptors. Antibodies of the invention may also be fused to albumin (including but not limited to recombinant human serum albumin (see, e.g., U.S. Pat. No. 5,876,969, issued Mar. 2, 1999, EP Patent 0 413 622, and U.S. Pat. No. 5,766,883, issued Jun. 16, 1998, herein incorporated by reference in their entirety)), resulting in chimeric polypeptides. In a preferred embodiment, polypeptides and/or antibodies of the present invention (including fragments or variants thereof) are fused with the mature form of human serum albumin (i.e., amino acids 1-585 of human serum albumin as shown in FIGS. 1 and 2 of EP Patent 0 322 094) which is herein incorporated by reference in its entirety. In another preferred embodiment, polypeptides and/or antibodies of the present invention (including fragments or variants thereof) are fused with polypeptide fragments comprising, or alternatively consisting of, amino acid residues 1-z of human serum albumin, where z is an integer from 369 to 419, as described in U.S. Pat. No. 5,766,883 herein incorporated by reference in its entirety. Polypeptides and/or antibodies of the present invention (including fragments or variants thereof) may be fused to either the N- or C-terminal end of the heterologous protein (e.g., immunoglobulin Fe polypeptide or human serum albumin polypeptide). Polynucleotides encoding fusion proteins of the invention are also encompassed by the invention. Such fusion proteins may, for example, facilitate purification and may increase half-life in vivo. Antibodies fused or conjugated to heterologous polypeptides may also be used in in vitro immunoassays and purification methods using methods known in the art. See e.g., Harbor et al., supra, and PCT publication WO 93/2 1232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452 (1991), which are incorporated by reference in their entireties.

The present invention further includes compositions comprising, or alternatively consisting of, heterologous polypeptides fused or conjugated to antibody fragments. For example, the heterologous polypeptides may be fused or conjugated to a Fab fragment, Fd fragment, Fv fragment, F(ab)₂ fragment, or a portion thereof. Methods for fusing or conjugating polypeptides to antibody portions are known in the art. See, e.g., U.S. Pat. Nos. 5,356,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88: 10535-10539 (1991); Zheng et al., J. Immunol. 154:5590-5600 (1995); and Vil et al., Proc. Natl. Acad. Sci. USA 89:11357-11341 (1992) (said references incorporated by reference in their entireties).

Additional fusion proteins of the invention may be generated through the techniques of gene-shuffling, motif-shuffling, exon-shuffling, and/or codon-shuffling (collectively referred to as “DNA shuffling”). DNA shuffling may be employed to modulate the activities of antibodies (including molecules comprising, or alternatively consisting of, antibody fragments or variants thereof), such methods can be used to generate antibodies with altered activity (e.g., antibodies with higher affinities and lower dissociation rates). See, generally, U.S. Pat. Nos. 5,605,793; 5,811,238; 5,830,721; 5,834,252; and 5,837,458, and Patten et al., Curr. Opinion Biotechnol. 8:724-35 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, polynucleotides encoding antibodies of the invention may be altered by being subjected to random mutagenesis by error-prone PCR, random nucleotide insertion or other methods prior to recombination. In another embodiment, one or more portions of a polynucleotide encoding an antibody which portions immunospecifically bind to TRAIL receptor may be recombined with one or more components, motifs, sections, parts, domains, fragments, etc. of one or more heterologous molecules.

Moreover, the antibodies of the present invention (including antibody fragments or variants thereof), can be fused to marker sequences, such as a polypeptides to facilitate purification. In preferred embodiments, the marker amino acid sequence is a hexa-histidine polypeptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., Cell 37:767 (1984)) and the FLAG® tag (Stratagene, La Jolla, Calif.).

The present invention further encompasses antibodies (including antibody fragments or variants thereof), conjugated to a diagnostic or therapeutic agent. The antibodies can be used diagnostically to, for example, monitor or prognose the development or progression of a tumor as part of a clinical testing procedure to, e.g., determine the efficacy of a given treatment regimen. Detection can be facilitated by coupling the antibody to a detectable substance. Examples of detectable substances include, but are not limited to, various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include, but are not limited to, horseradish peroxidase, alkaline phosphatase, beta-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythfin; an example of a luminescent material includes, but is not limited to, luminol; examples of bioluminescent materials include, but are not limited to, luciferase, luciferin, and aequorin; and examples of suitable radioactive material include, but are not limited to, iodine (¹¹¹I, ¹²³I, ¹²⁵I, ¹³¹I), carbon (¹⁴C), sulfur (³⁵S), tritium (³H), indium (¹¹¹In, ¹¹²In, ^{113m}In, ^{115m}In), technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³⁵Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ₁₈₈Re, ¹⁴²Pr, ¹⁰⁵Rh, and ⁹⁷Ru.

Further, an antibody of the invention (including an scFv or other molecule comprising, or alternatively consisting of, antibody fragments or variants thereof), may be coupled or conjugated to a therapeutic moiety such as a cytotoxin, e.g., a cytostatic or cytocidal agent, a therapeutic agent or a radioactive metal ion, e.g., alpha-emitters such as, for example, ²¹³Bi, or other radioisotopes such as, for example, ¹⁰³Pd, ¹³⁵Xe, ¹³¹I, ⁶⁸Ge, ⁵⁷Co, ⁶⁵Zn, ⁸⁵Sr, ³²P, ³⁵S, ⁹⁰Y, ¹⁵³Sm, ¹⁵³Gd, ¹⁶⁹Yb, ⁵¹Cr, ⁵⁴Mn, ⁷⁵Se, ¹¹³Sn, ⁹⁰Y, ¹¹⁷Tin, ¹⁸⁶Re, ¹⁸⁸Re and ¹⁶⁶Ho. In specific embodiments, an antibody or fragment thereof is attached to macrocyclic chelators that chelate radiometal ions, including but not limited to, ¹⁷⁷Lu, ⁹⁰Y, ¹⁶⁶Ho, and ¹⁵³Sm, to polypeptides. In specific embodiments, the macrocyclic chelator is 1,4,7,10-tetraazacyclododecane-N,N′,N″,N′″-tetraacetic acid (DOTA). In other specific embodiments, the DOTA is attached to the an antibody of the invention or fragment thereof via a linker molecule. Examples of linker molecules useful for conjugating DOTA to a polypeptide are commonly known in the art—see, for example, DeNardo et al., Clin Cancer Res. 4(10):2483-90, 1998; Peterson et al., Bioconjug. Chem. 10(4):553-7, 1999; and Zimmerman et al., Nucl. Med. Biol. 26(8):943-50, 1999 which are hereby incorporated by reference in their entirety.

A cytotoxin or cytotoxic agent includes any agent that is detrimental to cells. Examples include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, thymidine kinase, endonuclease, RNAse, and puromycin and frragments, variants or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Techniques known in the art may be applied to label antibodies of the invention. Such techniques include, but are not limited to, the use of bifunctional conjugating agents (see e.g., U.S. Pat. Nos. 5,756,065; 5,714,711; 5,696,239; 5,652,371; 5,505,931; 5,489,425; 5,435,990; 5,428,139; 5,342,604; 5,274,119; 4,994,560; and 5,808,003; the contents of each of which are hereby incorporated by reference in its entirety) and direct coupling reactions (e.g., Bolton-Hunter and Chloramine-T reaction).

The antibodies of the invention which are conjugates can be used for modifying a given biological response, the therapeutic agent or drug moiety is not to be construed as limited to classical chemical therapeutic agents. For example, the drug moiety may be a protein or polypeptide possessing a desired biological activity. Such proteins may include, but are not limited to, for example, a toxin such as abrin, ricin A, alpha toxin, pseudomonas exotoxin, or diphtheria toxin, saporin, momordin, gelonin, pokeweed antiviral protein, alpha-sarcin and cholera toxin; a protein such as tumor necrosis factor, alpha-interferon, beta-interferon, nerve growth factor, platelet derived growth factor, tissue plasminogen activator, an apoptotic agent, e.g., TNF-alpha, TNF-beta, AIM I (see, International Publication No. WO 97/35899), AIM II (see, International Publication No. WO 97/34911), Fas Ligand (Takahashi et al., Int. Immunol., 6:1567-1574 (1994)), VEGI (see, International Publication No. WO 99/23105), a thrombotic agent or an anti-angiogenic agent, e.g., angiostatin or endostatin; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

In specific embodiments antibodies of the invention are conjugated with a a polypeptide cytotoxin. An example of a suitable polypeptide cytotoxin is a ribosome-inactivating protein. Type I ribosome-inactivating proteins are single-chain proteins, while type 11 ribosome-inactivating proteins consist of two nonidentical subunits (A and B chains) joined by a disulfide bond (for a review, see Soria et al., Targeted Diagn. Ther. 7:193 (1992)). Useful type I ribosome-inactivating proteins include polypeptides from Saponaria officinalis (e.g., saporin-1, saporin-2, saporin-3, saporin-6), Momordica charantia (e.g. momordin), Byronia dioica (e.g., bryodin, bryodin-2), Trichosanthes kirilowii (e.g., trichosanthin, trichokirin), Gelonium multiflorum (e.g., gelonin), Phytolacca americana (e.g., pokeweed antiviral protein, pokeweed antiviral protein-II, pokeweed antiviral protein-S), Phytolacca dodecandra (e.g., dodecandrin, Mirabilis antiviral protein), and the like. Ribosome-inactivating proteins are described, for example, by Walsh et al., U.S. Pat. No. 5,635,384. Suitable type 11 ribosome-inactivating proteins include polypeptides from Ricinus conamunis (e.g., ricin), Abrus precatorius (e.g., abrin), Adenia digitata (e.g., modeccin), and the like. Since type II ribosome-inactiving proteins include a B chain that binds galactosides and a toxic A chain that depurinates adensoine, type 11 ribosome-inactivating protein conjugates should include the A chain. Additional useful ribosome-inactivating proteins include bouganin, clavin, maize ribosome-inactivating proteins, Vaccaria pyramidata ribosome-inactivating proteins, nigrine b, basic nigrine 1, ebuline, racemosine b, luffin-a, luffin-b, luffin-S, and other ribosome-inactivating proteins known to those of skill in the art. See, for example, Bolognesi and Stirpe, international publication No. WO98/55623, Colnaghi et al., international publication No. WO97/49726, Hey et al., U.S. Pat. No. 5,635,384, Bolognesi and Stirpe, international publication No. WO95/07297, Arias et al., international publication No. WO94/20540, Watanabe et al., J. Biochem. 106:6 977 (1989); Islam et al., Agric. Biol. Chem. 55:229 (1991), and Gao et al., FEBS Len. 347:257 (1994).

Antibodies of the invention (including antibody fragments or variants thereof), may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating a therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody of the invention can be conjugated to a second antibody to form an antibody heteroconjugate as described by Segal in U.S. Pat. No. 4,676,980, which is incorporated herein by reference in its entirety.

An antibody of the invention (including an other molecules comprising, or alternatively consisting of, an antibody fragment or variant thereof), with or without a therapeutic moiety conjugated to it, administered alone or in combination with cytotoxic factor(s) and/or cytokine(s) can be used as a therapeutic.

Uses of Antibodies of the Invention

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of TRAIL receptor polypeptides in biological samples. See, e.g., Harlow et al., Antibodies: A Laboratory Manual, (Cold Spring Harbor Laboratory Press, 2nd ed. 1988) (incorporated by reference herein in its entirety).

Immunophenotyping

The antibodies of the invention may be utilized for immunophenotyping of cell lines and biological samples (See, for example, Example 4). The translation product of the gene of the present invention may be useful as a cell specific marker, or more specifically as a cellular marker that is differentially expressed at various stages of differentiation and/or maturation of particular cell types, particularly of tumors and cancer cells. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

Epitope Mapping

The present invention provides antibodies (including antibody fragments or variants thereof), that can be used to identify epitopes of a TRAIL receptor polypeptide. In particular, the antibodies of the present invention can be used to identify epitopes of a human TRAIL receptor polypeptide (e.g., SEQ ID NOS:1-4) or a TRAIL receptor polypeptide expressed on human cells; a murine TRAIL receptor or a TRAIL receptor polypeptide expressed on murine cells; a rat TRAIL receptor polypeptide receptor or a TRAIL receptor polypeptide expressed on rat cells; or a monkey TRAIL receptor polypeptide or a TRAIL receptor polypeptide expressed on monkey cells, using techniques described herein or otherwise known in the art. Fragments which function as epitopes may be produced by any conventional means. (See, e.g., Houghten, Proc. Natl. Acad. Sci. USA 82:5131-5135 (1985), further described in U.S. Pat. No. 4,711,211.) Identified epitopes of antibodies of the present invention may, for example, be used as vaccine candidates, i.e., to immunize an individual to elicit antibodies against the naturally occuring forms of TRAIL receptor polypeptides.

Diagnostic Uses of Antibodies

Labeled antibodies of the invention (including molecules comprising, or alternatively consisting of, antibody fragments or variants thereof) which specifically bind to a TRAIL receptor polypeptide can be used for diagnostic purposes to detect, diagnose, prognose, or monitor diseases and/or disorders. In specific embodiments, labeled antibodies of the invention (including molecules comprising, or alternatively consisting of, antibody fragments or variants thereof) which specifically bind to a TRAIL receptor polypeptide can be used for diagnostic purposes to detect, diagnose, prognose, or monitor diseases and/or disorders associated with the aberrant expression and/or activity of a TRAIL receptor polypeptide or a TRAIL receptor polypeptide receptor.

The invention provides for the detection of expression of a TRAIL receptor polypeptide comprising: (a) assaying the expression of a TRAIL receptor polypeptide in a biological sample from an individual using one or more antibodies of the invention that immunospecifically binds to a TRAIL receptor polypeptide; and (b) comparing the level of a TRAIL receptor polypeptide with a standard level of a TRAIL receptor polypeptide, (e.g., the level in normal biological samples).

The invention provides for the detection of aberrant expression of a TRAIL receptor polypeptide comprising: (a) assaying the expression of a TRAIL receptor polypeptide in a biological sample from an individual using one or more antibodies of the invention that immunospecifically binds to a TRAIL receptor polypeptide; and (b) comparing the level of a TRAIL receptor polypeptide with a standard level of a TRAIL receptor polypeptide, e.g., in normal biological samples, whereby an increase or decrease in the assayed level of a TRAIL receptor polypeptide compared to the standard level of a TRAIL receptor polypeptide is indicative of aberrant expression.

By “biological sample” is intended any fluids and/or cells obtained from an individual, body fluid, body tissue, body cell, cell line, tissue culture, or other source which may contain a TRAIL receptor polypeptide protein or mRNA. Body fluids include, but are not limited to, sera, plasma, urine, synovial fluid, spinal fluid, saliva, and mucous. Tissues samples may be taken from virtually any tissue in the body. Tissue samples may also be obtained from autopsy material. Methods for obtaining tissue biopsies and body fluids from mammals are well known in the art. Where the biological sample is to include mRNA, a tissue biopsy is the preferred source.

Antibodies of the invention (including molecules comprising, or alternatively consisting of, antibody fragments or variants thereof) which specifically bind to a TRAIL receptor polypeptide can be used for diagnostic purposes to detect, diagnose, prognose, or monitor cancers and other hyperproliferative disorders, and/or diseases or conditions associated therewith. The invention provides for the detection of aberrant expression of a TRAIL receptor polypeptide comprising: (a) assaying the expression of a TRAIL receptor polypeptide in a biological sample from an individual using one or more antibodies of the invention that immunospecifically binds to a TRAIL receptor polypeptide; and (b) comparing the level of a TRAIL receptor polypeptide with a standard level of a TRAIL receptor polypeptide, e.g., in normal biological samples, whereby an increase or decrease in the assayed level of a TRAIL receptor polypeptide compared to the standard level of a TRAIL receptor polypeptide is indicative of a cancer and/or a hyperproliferative disorder.

TRAIL has been shown in some instances to selectively kill tumor cells (See, for example, Oncogene 19:3363-71 (2000)). This may be a result of differential expression of TRAIL receptors on normal and cancerous cells. Thus, in specific embodiments, an increase in the assayed level of a TRAIL receptor polypeptide is indicative of a cancer and/or a hyperproliferative disorder.

Other reports suggest that decreased TRAIL receptor expression by tumor cells may be a mechanism by which tumor cells evade the immune system (See, for example, Int. J. Oncol. 16:917-25 (2000)) Thus, in other specific embodiments, a decrease in the assayed level of a TRAIL receptor polypeptide is indicative of a cancer and/or a hyperproliferative disorder.

One aspect of the invention is the detection and diagnosis of a disease or disorder associated with aberrant expression of a TRAIL receptor polypeptide or a TRAIL receptor polypeptide receptor in an animal, preferably a mammal and most preferably a human. In one embodiment, diagnosis comprises: a) administering (for example, parenterally, subcutaneously, or intraperitoneally) to a subject an effective amount of a labeled antibody of the invention (including molecules comprising, or alternatively consisting of, antibody fragments or variants thereof) that immunospecifically binds to a TRAIL receptor polypeptide; b) waiting for a time interval following the administering for permitting the labeled antibody to preferentially concentrate at sites in the subject where TRAIL receptor polypeptide is expressed (and for unbound labeled molecule to be cleared to background level); c) determining background level; and d) detecting the labeled antibody in the subject, such that detection of labeled antibody or fragment thereof above the background level and above or below the level observed in a person without the disease or disorder indicates that the subject has a particular disease or disorder associated with aberrant expression of a TRAIL receptor polypeptide or a TRAIL receptor polypeptide receptor. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99Tc. The labeled antibody will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In one embodiment, monitoring of the disease or disorder is carried out by repeating the method for diagnosing the disease or disorder, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

Therapeutic Uses of Antibodies

One or more antibodies of the present invention (including molecules comprising, or alternatively consisting of, antibody fragments or variants thereof) that immunospecifically bind to TRAIL receptor may be used locally or systemically in the body as a therapeutic. The present invention is further directed to antibody-based therapies which involve administering antibodies of the invention (including molecules comprising, or alternatively consisting of, antibody fragments or variants thereof) to an animal, preferably a mammal, and most preferably a human, for preventing or treating one or more of the disclosed diseases, disorders, or conditions. Therapeutic compounds of the invention include, but are not limited to, antibodies of the invention and nucleic acids encoding antibodies (and anti-idiotypic antibodies) of the invention as described herein. In one embodiment, the antibodies of the invention can be used to treat, ameliorate or prevent diseases, disorders or conditions, including, but not limited to, any one or more of the diseases, disorders, or conditions described herein. The treatment and/or prevention of diseases, disorders, or conditions includes, but is not limited to, alleviating symptoms associated with those diseases, disorders or conditions. Antibodies of the invention may be provided in pharmaceutically acceptable compositions as known in the art or as described herein. In certain embodiments, properties of the antibodies of the present invention, as detailed in the Examples below, make the antibodies better therapeutic agents than previously described TRAIL receptor binding antibodies.

Therapeutic Uses of Antibodies for Treating Cancers

In highly preferred embodiments, antibodies of the invention that bind a TRAIL receptor polypeptide and stimulate apoptosis of TRAIL receptor expressing cells are used to treat, prevent or ameliorate cancer. In other highly preferred embodiments, antibodies of the invention that bind a TRAIL receptor polypeptide are used to treat, prevent or ameliorate cancer. In specific embodiments, antibodies of the invention are used to inhibit the progression or metastasis of cancers and other related disorders. Cancers and related disorders, include, but are not limited to, colon cancer, cervical cancer, leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In highly preferred embodiments, antibodies of the invention that bind TR4 and stimulate apoptosis of TR4 expressing cells are used to treat, prevent or ameliorate renal cancer.

In other preferred embodiments, antibodies of the invention that bind TR4 are used to treat, prevent or ameliorate renal cancer.

In highly preferred embodiments, antibodies of the invention that bind TR4 and stimulate apoptosis of TR4 expressing cells are used to treat, prevent or ameliorate melanoma.

In other preferred embodiments, antibodies of the invention that bind TR4 are used to treat, prevent or ameliorate melanoma.

In highly preferred embodiments, antibodies of the invention that bind TR4 and stimulate apoptosis of TR4 expressing cells are used to treat, prevent or ameliorate cancers of the liver such as hepatomas.

In other preferred embodiments, antibodies of the invention that bind TR4 are used to treat, prevent or ameliorate cancers of the liver such as hepatomas.

In other highly preferred embodiments, antibodies of the invention that bind TR4 and stimulate apoptosis of TR4 expressing cells are used to treat, prevent or ameliorate leukemia.

In other preferred embodiments, antibodies of the invention that bind TR4 are used to treat, prevent or ameliorate leukemia.

In other highly preferred embodiments, antibodies of the invention that bind TR4 and stimulate apoptosis of TR4 expressing cells are used to treat, prevent or ameliorate chronic lymphocytic leukemia (CLL).

In other preferred embodiments, antibodies of the invention that bind TR4 are used to treat, prevent or ameliorate chronic lymphocytic leukemia (CLL).

In other highly preferred embodiments, antibodies of the invention that bind TR4 and stimulate apoptosis of TR4 expressing cells are used to treat, prevent or ameliorate myelodysplastic syndrome.

In other preferred embodiments, antibodies of the invention that bind TR4 are used to treat, prevent or ameliorate myelodysplastic syndrome.

In other highly preferred embodiments, antibodies of the invention that bind TR4 and stimulate apoptosis of TR4 expressing cells are used to treat, prevent or ameliorate bone cancers including but not limited to Ewing's sarcoma and osteosarcoma.

In other preferred embodiments, antibodies of the invention that bind TR4 are used to treat, prevent or ameliorate bone cancers including but not limited to Ewing's sarcoma and osteosarcoma.

In highly preferred embodiments, antibodies of the invention that bind TR4 and stimulate apoptosis of TR4 expressing cells are used to treat, prevent or ameliorate cancers of the central nervous system such as medulloblastoma, neuroblastoma, and glioblastoma.

In other preferred embodiments, antibodies of the invention that bind TR4 are used to treat, prevent or ameliorate cancers of the central nervous system such as medulloblastoma, neuroblastoma, and glioblastomaln other preferred embodiments, antibodies of the invention that bind TR4 are used to treat, prevent or hematological cancers such as multiple myeloma, non-Hodgkin's lymphoma, chronic lymphocytic leukemia and chronic myelgenous leukemia.

In highly preferred embodiments, antibodies of the invention that bind TR4 and stimulate apoptosis of TR4 expressing cells are used to treat, prevent or ameliorate multiple myeloma.

In highly preferred embodiments, antibodies of the invention that bind TR4 and stimulate apoptosis of TR4 expressing cells are used to treat, prevent or ameliorate multiple myeloma.

In other preferred embodiments, antibodies of the invention that bind TR4 are used to treat, prevent or ameliorate multiple myeloma.

In other highly preferred embodiments, antibodies of the invention that bind TR4 and stimulate apoptosis of TR4 expressing cells are used to treat, prevent or ameliorate non-Hodgkin's lymphoma.

In other preferred embodiments, antibodies of the invention that bind TR4 are used to treat, prevent or ameliorate non-Hodgkin's lymphoma. Non hodgkin's lymphomas, include but are not limited to, B cell lymphomas such as precursor B lymphoblastic lymphoma, small lymphocytic lymphoma, B-cell prolymphocytic lymphoma, lymphoplasmacytic lymphoma, splenic marginal zone lymphoma, extranodal marginal zone—MALT lymphoma, nodal marginal zone lymphoma, follicular lymphoma, mantle cell lymphoma, diffuse large B-cell lymphoma, primary mediastinal large B-cell lymphoma, primary effusion lymphoma and Burkitt's lymphoma) and T-cell lymphomas such as precursor (peripheral) T-cell lymphoblastic lymphoma, adult T-cell lymphoma, extranodal Natural Killer/T-cell, nasal type lymphoma, enteropathy type T-cell lymphoma, hepatosplenic T-cell lymphoma, subcutaneous panniculitis like T-cell lymphoma, skin (cutaneous) lymphomas (including mycosis fungoides and Sezary syndrome), anaplastic large cell lymphoma, peripheral T-cell, not otherwise specified lymphoma, and angioimmunoblastic T-cell lymphoma.

In other highly preferred embodiments, antibodies of the invention that bind TR4 and stimulate apoptosis of TR4 expressing cells are used to treat, prevent or ameliorate chronic lymphocytic leukemia (CLL).

In other preferred embodiments, antibodies of the invention that bind TR4 are used to treat, prevent or ameliorate chronic lymphocytic leukemia (CLL).

In other highly preferred embodiments, antibodies of the invention that bind TR4 and stimulate apoptosis of TR4 expressing cells are used to treat prevent or ameliorate chronic myelogenous leukemia (CML).

In other preferred embodiments, antibodies of the invention that bind TR4 are used to treat, prevent or ameliorate chronic myelogenous leukemia (CML).

In highly preferred embodiments, antibodies of the invention that bind TR4 and stimulate apoptosis of TR4 expressing cells are used to treat, prevent or ameliorate prostate cancer and/or metastatic prostate cancer.

In other preferred embodiments, antibodies of the invention that bind TR4 are used to treat, prevent or ameliorate prostate cancer and/or metastatic prostate cancer

It has been demonstrated, in accordance with the present invention that the expression of TRAIL receptor on lung carcinoma tissue, bladder carcinoma tissue and Ovarian carcinoma tissue. Additionally, it has been demonstrated, in accordance with the present invention that TRAIL receptor is expressed on primary breast, colon, lung, and stomach tumor tissue. (See, Example 7).

Thus, in highly preferred embodiments, antibodies of the invention that bind a TRAIL receptor polypeptide and stimulate apoptosis of TRAIL receptor expressing cells are used to treat lung cancer, including but not limited to non-small cell lung cancer.

In other preferred embodiments, antibodies of the invention that bind a TRAIL receptor polypeptide are used to treat lung cancer, including but not limited to non-small cell lung cancer.

In other highly preferred embodiments, antibodies of the invention that bind a TRAIL receptor polypeptide and stimulate apoptosis of TRAIL receptor expressing cells are used to treat bladder cancer.

In other preferred embodiments, antibodies of the invention that bind a TRAIL receptor polypeptide are used to treat bladder cancer.

In other highly preferred embodiments, antibodies of the invention that bind a TRAIL receptor polypeptide and stimulate apoptosis of TRAIL receptor expressing cells are used to treat ovarian cancer.

In other preferred embodiments, antibodies of the invention that bind a TRAIL receptor polypeptide are used to treat ovarian cancer.

In other highly preferred embodiments, antibodies of the invention that bind a TRAIL receptor polypeptide and stimulate apoptosis of TRAIL receptor expressing cells are used to treat breast cancer and/or breast cancers that have metastasized.

In other preferred embodiments, antibodies of the invention that bind a TRAIL receptor polypeptide are used to treat breast cancer and/or breast cancers that have metastasized.

In other highly preferred embodiments, antibodies of the invention that bind a TRAIL receptor polypeptide and stimulate apoptosis of TRAIL receptor expressing cells are used to treat colon cancer or colo-rectal cancer.

In other preferred embodiments, antibodies of the invention that bind a TRAIL receptor polypeptide are used to treat colon cancer or colo-rectal cancer.

In other highly preferred embodiments, antibodies of the invention that bind a TRAIL receptor polypeptide and stimulate apoptosis of TRAIL receptor expressing cells are used to treat stomach cancer.

In other preferred embodiments, antibodies of the invention that bind a TRAIL receptor polypeptide are used to treat stomach cancer.

In another embodiment, antibodies of the invention that bind a TRAIL receptor polypeptide and optionally, stimulate apoptosis of TRAIL receptor expressing cells, are used to treat diseases and/or disorders associated with increased cell survival, or the inhibition of apoptosis, including cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostrate cancer, Kaposi's sarcoma and ovarian cancer); autoimmune disorders (such as multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis rheumatoid arthritis) and viral infections (such as herpes viruses, pox viruses and adenoviruses), information graft v. host disease, acute graft rejection, and chronic graft rejection. In preferred embodiments, the antibodies and antibody compositions of the invention are used to inhibit growth, progression, and/or metastasis of cancers, in particular those listed above. In preferred embodiments the antibodies and antibody compositions of the invention are not hepatotoxic, in vitro or in vivo.

In preferred embodiments, the antibodies of the invention that are used to treat, prevent or ameliorate the cancers described above specifically and/or preferentially bind TR4. In other preferred embodiments, the antibodies of the invention that are used to treat, prevent or ameliorate the cancers described above specifically and/or preferentially bind TR4 and TR7.

In preferred embodiments, the antibodies of the invention are used to treat, prevent or ameliorate radiation resistant cancers and/or cancers that are resistant to one or more chemotherapeutic agents or other therapeutic agents useful in the treatment of cancers.

In another preferred embodiment, the antibodies of the invention are used to treat, prevent or ameliorate premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79.)

Hyperplasia is a form of controlled cell proliferation, involving an increase in cell number in a tissue or organ, without significant alteration in structure or function. Hyperplastic disorders which can be diagnosed, prognosed, prevented, and/or treated with compositions of the invention (including polynucleotides, polypeptides, agonists or antagonists) include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

Metaplasia is a form of controlled cell growth in which one type of adult or fully differentiated cell substitutes for another type of adult cell. Metaplastic disorders which can be diagnosed, prognosed, prevented, and/or treated with compositions of the invention (including polynucleotides, polypeptides, agonists or antagonists) include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be diagnosed, prognosed, prevented, and/or treated with compositions of the invention (including polynucleotides, polypeptides, agonists or antagonists) include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be diagnosed, prognosed, prevented, and/or treated with compositions of the invention (including polynucleotides, polypeptides, agonists or antagonists) include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

Additional Therapeutic Uses of Antibodies

In another embodiment, the invention provides methods and compositions for inhibiting the growth of or killing TRAIL receptor expressing cells, comprising, or alternatively consisting of, administering to an animal in which such inhibition of growth or killing of TRAIL receptor expressing cells is desired, antibody or antibody compositions of the invention (e.g., antibody fragments and variants, antibody mixtures, antibody multimers, fusion proteins of the invention, and antibodies in combination with other therapeutic compounds such as chemotherapeutic agents) in an amount effective to inhibit the growth of or kill TRAIL receptor expressing cells.

In one aspect, the present invention is directed to a method for enhancing apoptosis induced by a TNF-family ligand (especially TRAIL (SEQ ID NO:5)), which involves contacting a cell which expresses a TRAIL receptor polypeptide with an effective amount of an antibody or antibody composition of the invention, preferably an agonistic anti-TR4, anti-TR7, and/or an anti-TR4 and TR7 antibody (i.e., an antibody that immunospecifically binds both TR4 and TR7), capable of inducing or increasing TRAIL receptor mediated signaling, especially TR4 and TR7 mediated signalling. Preferably, TRAIL receptor mediated signaling is increased or induced by an antibody of the invention to treat a disease wherein decreased apoptosis or decreased cytokine and adhesion molecule expression is exhibited.

In one aspect, the present invention is directed to a method for inducing apoptosis of TR4 and/or TR7 expressing cells, which involves contacting a cell which expresses TR4 and/or TR7, with an effective amount of an antibody or antibody composition of the invention, preferably an agonistic anti-TR4, anti-TR7, and/or an anti-TR4 and TR7 antibody (i.e., an antibody that immunospecifically binds both TR4 and TR7), capable of inducing or increasing TRAIL receptor mediated signaling, especially TR4 and TR7 mediated signalling.

In a further aspect, the present invention is directed to a method for inhibiting apoptosis induced by a TNF-family ligand (especially TRAIL (SEQ ID NO:5)), which involves contacting a cell which expresses a TRAIL receptor polypeptide, with an effective amount of an antibody or antibody composition of the invention, preferably an antagonistic anti-TR4, anti-TR7 antibody, and/or an anti-TR4 and TR7 antibody (i.e., an antibody that immunospecifically binds both TR4 and TR7), capable of decreasing TRAIL receptor mediated signaling, especially TR4 and TR7 mediated signalling. Preferably, TRAIL receptor mediated signaling is decreased to treat a disease wherein increased apoptosis or NFκB expression is exhibited.

In one aspect, the present invention is directed to a method for inhibiting apoptosis of TR4 and/or TR7 expressing cells, which involves contacting a cell which expresses TR4 and/or TR7, with an effective amount of an antibody or antibody composition of the invention, preferably an antagonistic anti-TR4, anti-TR7, and/or an anti-TR4 and TR7 antibody (i.e., an antibody that immunospecifically binds both TR4 and TR7), capable of decreasing TRAIL receptor mediated signaling, especially TR4 and TR7 mediated signalling.

By TRAIL receptor “agonist” is intended naturally occurring and synthetic compounds capable of enhancing or potentiating apoptosis mediated by TRAIL receptor. By TRAIL receptor “antagonist” is intended naturally occurring and synthetic compounds capable of inhibiting apoptosis mediated by TRAIL receptor. Whether any candidate TRAIL receptor “agonist” or “antagonist” of the present invention can enhance or inhibit, respectively, apoptosis can be determined using art-known TNF-family ligand/receptor cellular response assays, including those described in more detail below.

The antibodies of the invention can be used to treat, ameliorate or prevent diseases, disorders or conditions associated with aberrant expression and/or activity of TRAIL receptor or TRAIL receptor ligand, including, but not limited to, any one or more of the diseases, disorders, or conditions described herein. The treatment and/or prevention of diseases, disorders, or conditions associated with aberrant TRAIL receptor expression and/or activity or aberrant TRAIL receptor ligand expression and/or activity includes, but is not limited to, alleviating symptoms associated with those diseases, disorders or conditions. Antibodies of the invention may be provided in pharmaceutically acceptable compositions as known in the art or as described herein.

Further, antibodies of the present invention (including molecules comprising, or alternatively consisting of, antibody fragments or variants thereof) which activate TRAIL receptor-mediated biological activities (e.g., the induction of apoptosis in TRAIL recpetor expressing cells) can be administered to an animal to treat, prevent or ameliorate a disease or disorder described herein, particularly cancers and other hyperproliferative disorders. These antibodies may potentiate or activate either all or a subset of the biological activities of TRAIL receptor, for example, by inducing a conformational change in TRAIL receptor. In a specific embodiment, an antibody of the present invention that increases TRAIL receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to TRAIL receptor activity in absence of the antibody is administered to an animal to treat, prevent or ameliorate a disease or disorder. In another embodiment, a combination of antibodies, a combination of antibody fragments, a combination of antibody variants, or a combination of antibodies, antibody fragments and/or antibody variants that increase TRAIL receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to TRAIL receptor activity in absence of the said antibodies or antibody fragments and/or antibody variants is administered to an animal to treat, prevent or ameliorate a disease or disorder.

Further, antibodies of the present invention (including molecules comprising, or alternatively consisting of, antibody fragments or variants thereof) which activate TRAIL receptor-mediated biological activities (e.g., the induction of apoptosis in TRAIL receptor expressing cells) can be administered to an animal to treat, prevent or ameliorate a disease or disorder associated with aberrant TRAIL receptor expression, lack of TRAIL receptor function, aberrant TRAIL receptor ligand expression, or lack of TRAIL receptor ligand function. These antibodies may potentiate or activate either all or a subset of the biological activities of TRAIL receptor, for example, by inducing a conformational change in TRAIL receptor. In a specific embodiment, an antibody of the present invention that increases TRAIL receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to TRAIL receptor activity in absence of the antibody is administered to an animal to treat, prevent or ameliorate a disease or disorder associated with aberrant TRAIL receptor expression, lack of TRAIL receptor function, aberrant TRAIL receptor ligand expression, or lack of TRAIL receptor ligand function. In another embodiment, a combination of antibodies, a combination of antibody fragments, a combination of antibody variants, or a combination of antibodies, antibody fragments and/or antibody variants that increase TRAIL receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to TRAIL receptor activity in absence of the said antibodies or antibody fragments and/or antibody variants is administered to an animal to treat, prevent or ameliorate a disease or disorder associated with aberrant TRAIL receptor expression or lack of TRAIL receptor function or aberrant TRAIL receptor ligand expression or lack of TRAIL receptor ligand function.

Antibodies of the present invention (including molecules comprising, or alternatively consisting of, antibody fragments or variants thereof) that function as agonists or antagonists of a TRAIL receptor, preferably of TRAIL receptor signal transduction, can be administered to an animal to treat, prevent or ameliorate a disease or disorder associated with aberrant TRAIL receptor expression, lack of TRAIL receptor function, aberrant TRAIL receptor ligand expression, or lack of TRAIL receptor ligand function. For example, antibodies of the invention which mimic the action of TRAIL binding to the TRAIL receptor, in full or in part, (e.g. antibodies that act as TRAIL receptor agonists), may be administered to an animal to treat, prevent or ameliorate a disease or disorder associated with aberrant TRAIL receptor expression, lack of TRAIL receptor function, aberrant TRAIL receptor ligand expression, or lack of TRAIL receptor ligand function. As an alternative example, antibodies of the invention which disrupt or prevent the interaction between TRAIL receptor and its ligand or inhibit, reduce, or prevent signal transduction through one or more TRAIL receptors, may be administered to an animal to treat, prevent or ameliorate a disease or disorder associated with aberrant TRAIL receptor expression, lack of TRAIL receptor function, aberrant TRAIL receptor ligand expression, or lack of TRAIL receptor ligand function. Antibodies of the invention which do not prevent a TRAIL receptor from binding its ligand but inhibit or downregulate TRAIL receptor signal transduction can be administered to an animal to treat, prevent or ameliorate a disease or disorder associated with aberrant TRAIL receptor expression, lack of TRAIL receptor function, aberrant TRAIL receptor ligand expression, or lack of TRAIL receptor ligand function. The ability of an antibody of the invention to enhance, inhibit, upregulate or downregulate TRAIL receptor signal transduction may be determined by techniques described herein or otherwise known in the art. For example, TRAIL-induced receptor activation and the activation of signaling molecules can be determined by detecting the association of adaptor proteins such as FADD and TRADD with the TRAIL receptors, by immunoprecipitation followed by western blot analysis (for example, as described herein).

Further, antibodies of the present invention (including molecules comprising, or alternatively consisting of, antibody fragments or variants thereof) which activate TRAIL receptor-mediated biological activities (e.g., the induction of apoptosis in TRAIL receptor expressing cells) can be administered to an animal to treat, prevent or ameliorate a disease or disorder associated with aberrant TRAIL receptor expression, lack of TRAIL receptor function, aberrant TRAIL receptor ligand expression, or lack of TRAIL receptor ligand function. These antibodies may potentiate or activate either all or a subset of the biological activities of TRAIL receptor, for example, by inducing a conformational change in TRAIL receptor. In a specific embodiment, an antibody of the present invention that increases TRAIL receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to TRAIL receptor activity in absence of the antibody is administered to an animal to treat, prevent or ameliorate a disease or disorder associated with aberrant TRAIL receptor expression, lack of TRAIL receptor function, aberrant TRAIL receptor ligand expression, or lack of TRAIL receptor ligand function. In another embodiment, a combination of antibodies, a combination of antibody fragments, a combination of antibody variants, or a combination of antibodies, antibody fragments and/or antibody variants that increase TRAIL receptor activity by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least two-fold, at least three-fold, at least four fold, at least five fold, at least ten-fold, at least twenty-fold, at least fifty-fold, or at least one hundred-fold relative to TRAIL receptor activity in absence of the said antibodies or antibody fragments and/or antibody variants is administered to an animal to treat, prevent or ameliorate a disease or disorder associated with aberrant TRAIL receptor expression or lack of TRAIL receptor function or aberrant TRAIL receptor ligand expression or lack of TRAIL receptor ligand function.

In a specific embodiment, an antibody of the present invention (including molecules comprising, or alternatively consisting of, antibody fragments or variants thereof) that inhibits or downregulates, in full or in part, TRAIL receptor activity (e.g., stimulation of apoptosis) by at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to TRAIL receptor activity in absence of the antibody is administered to an animal to treat, prevent or ameliorate a disease or disorder associated with aberrant TRAIL receptor expression, excessive TRAIL receptor function, aberrant TRAIL receptor ligand expression, or excessive TRAIL receptor ligand function. In another embodiment, a combination of antibodies, a combination of antibody fragments, a combination of antibody variants, or a combination of antibodies, antibody fragments, and/or variants that inhibit or downregulate TRAIL receptor activity by at least 95%, at least 90%, at least 85%, at least 80%, at least 75%, at least 70%, at least 65%, at least 60%, at least 55%, at least 50%, at least 45%, at least 40%, at least 45%, at least 35%, at least 30%, at least 25%, at least 20%, or at least 10% relative to TRAIL receptor activity in absence of said antibodies, antibody fragments, and/or antibody variants are administered to an animal to treat, prevent or ameliorate a disease or disorder associated with aberrant TRAIL receptor expression, excessive TRAIL receptor function, aberrant TRAIL receptor ligand expression, or excessive TRAIL receptor ligand function.

In one embodiment, therapeutic or pharmaceutical compositions of the invention are administered to an animal to treat, prevent or ameliorate a disease or disorder diseases associated with increased apoptosis including, but not limited to, AIDS, neurodegenerative disorders (such as Alzheimer's disease, Parkinson's disease, Amyotrophic lateral sclerosis, Retinitis pigmentosa, Cerebellar degeneration), myelodysplastic syndromes (such as aplastic anemia), ischemic injury (such as that caused by myocardial infarction, stroke and reperfusion injury), toxin-induced liver disease (such as that caused by alcohol), septic shock, cachexia and anorexia In another embodiment, therapeutic or pharmaceutical compositions of the invention are administered to an animal to treat, prevent or ameliorate bone marrow failure, for example, aplastic anemia and myelodysplastic syndrome.

Therapeutic or pharmaceutical compositions of the invention, may also be administered to treat, prevent, or ameliorate organ rejection or graft-versus-host disease (GVHD) and/or conditions associated therewith. Organ rejection occurs by host immune cell destruction of the transplanted tissue through an immune response. Similarly, an immune response is also involved in GVHD, but, in this case, the foreign transplanted immune cells destroy the host tissues. Cellular death induced by immune cell effector functions is apoptotic death. Thus, the administration of antibodies of the invention, (e.g., those that inhibit apoptosis), may be an effective therapy in preventing organ rejection or GVHD.

In another embodiment, therapeutic or pharmaceutical compositions of the invention are administered to an animal to treat, prevent or ameliorate infectious diseases. Infectious diseases include diseases associated with yeast, fungal, viral and bacterial infections. Viruses associated with viral infections which can be treated or prevented in accordance with this invention include, but are not limited to, retroviruses (e.g., human T-cell lymphotrophic virus (HTLV) types 1 and 11 and human immunodeficiency virus (HIV)), herpes viruses (e.g., herpes simplex virus (HSV) types I and II, Epstein-Barr virus, HHV6-HHV8, and cytomegalovirus), arenavirues (e.g., lassa fever virus), paramyxoviruses (e.g., morbillivirus virus, human respiratory syncytial virus, mumps, and pneumovirus), adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., influenza viruses A, B and C), papovaviruses (e.g., papillomavirues), picomaviruses (e.g., rhinoviruses, enteroviruses and hepatitis A viruses), poxviruses, reoviruses (e.g., rotavirues), togaviruses (e.g., rubella virus), rhabdoviruses (e.g., rabies virus). Microbial pathogens associated with bacterial infections include, but are not limited to, Streptococcus pyogenes, Streptococcus pneumoniae, Neisseria gonorrhoea, Neisseria meningitides, Corynebactenum diphtheriae, Clostridium botulinum, Clostridium perfringens, Clostridium tetani, Haemophilus influencae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersiniapseudotuberculosis, Shigella dysenteriae, Shigella flexneri, Shigella sonnei, Salmonella typhimurium, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlandyia spp., and Helicobacter pylori.

In another embodiments, antibodies and antibody compositions of the present invention are used to treat, prevent, or ameliorate diseases associated with increased apoptosis including, but not limited to, AIDS, neurodegenerative disorders (such as Alzheimer's disease, Parkinson's disease, Amyotrophic lateral sclerosis, Retinitis pigmentosa, Cerebellar degeneration), brain tumor or prion associated disease); autoimmune disorders (such as, multiple sclerosis, Rheumatoid Arthritis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as aplastic anemia), graft v. host disease, ischemic injury (such as that caused by myocardial infarction, stroke and reperfusion injury), liver injury (e.g., hepatitis related liver injury, ischemia/reperfusion injury, cholestosis (bile duct injury) and liver cancer); toxin-induced liver disease (such as that caused by alcohol), septic shock, cachexia and anorexia. In preferred embodiments, TRAIL receptor antagonistic antibodies, (e.g., antibodies that bind one or more TRAIL receptor polypeptides, prevent TRAIL from binding to the TRAIL receptors to which the antibodies are bound, but do not transduce the biological signal that results in apoptosis) are used to treat the diseases and disorders listed above.

Many of the pathologies associated with HIV are mediated by apoptosis, including HIV-induced nephropathy and HIV encephalitis. Thus, in additional preferred embodiments, antibodies, preferably antagonistic TRAIL receptor antibodies, of the invention are used to treat AIDS and pathologies associated with AIDS. Another embodiment of the present invention is directed to the use of antibodies of the invention to reduce TRAIL-mediated death of T cells in HIV-infected patients.

In additional embodiments, antibodies of the present invention, particularly antagonistic anti-TRAIL Receptor antibodies, are administered in combination with other inhibitors of T cell apoptosis. For example, Fas-mediated apoptosis has been implicated in loss of T cells in HIV individuals (Katsikis et al., J. Exp. Med. 181:2029-2036, 1995). Thus, a patient susceptible to both Fas ligand mediated and TRAIL mediated T cell death may be treated with both an agent that blocks TRAIL/TRAIL receptor interactions and an agent that blocks Fas-ligand/Fas interactions. Suitable agents for blocking binding of Fas-ligand to Fas include, but are not limited to, soluble Fas polypeptides; mulitmeric forms of soluble Fas polypeptides (e.g., dimers of sFas/Fc); anti-Fas antibodies that bind Fas without transducing the biological signal that results in apoptosis; anti-Fas-ligand antibodies that block binding of Fas-ligand to Fas; and muteins of Fas-ligand that bind Fas but do not transduce the biological signal that results in apoptosis. Preferably, the antibodies employed according to this method are monoclonal antibodies. Examples of suitable agents for blocking Fas-ligand/Fas interactions, including blocking anti-Fas monoclonal antibodies, are described in International application publication number WO 95/10540, hereby incorporated by reference.

Suitable agents, which also block binding of TRAIL to a TRAIL receptor that may be administered with the antibodies of the present invention include, but are not limited to, soluble TRAIL receptor polypeptides (e.g., a soluble form of OPG, TR5 (International application publication number WO 98/30693); a soluble form of TR4 (International publication number WO 98/32856); TR7/DR5 (International application publication number WO 98/41629); and TR10 (International application publication number WO 98/54202)); multimeric forms of soluble TRAIL receptor polypeptides; and TRAIL receptor antibodies that bind the TRAIL receptor without transducing the biological signal that results in apoptosis, anti-TRAIL antibodies that block binding of TRAIL to one or more TRAIL receptors, and muteins of TRAIL that bind TRAIL receptors but do not transduce the biological signal that results in apoptosis.

In rejection of an allograft, the immune system of the recipient animal has not previously been primed to respond because the immune system for the most part is only primed by environmental antigens. Tissues from other members of the same species have not been presented in the same way that, for example, viruses and bacteria have been presented. In the case of allograft rejection, immunosuppressive regimens are designed to prevent the immune system from reaching the effector stage. However, the immune profile of xenograft rejection may resemble disease recurrence more that allograft rejection. In the case of disease recurrence, the immune system has already been activated, as evidenced by destruction of the native islet cells. Therefore, in disease recurrence the immune system is already at the effector stage. Antibodies of the present invention (e.g., agonistic antibodies of the invention) are able to suppress the immune response to both allografts and xenografts because lymphocytes activated and differentiated into effector cells will express the TRAIL receptor polypeptides, and thereby are susceptible to compounds which enhance apoptosis. Thus, the present invention further provides a method for creating immune privileged tissues. Antagonist of the invention can further be used in the treatment of Inflammatory Bowel-Disease.

Antibodies and antibody compositions of the invention may be useful for treating inflammatory diseases, such as rheumatoid arthritis, osteoarthritis, psoriasis, septicemia, and inflammatory bowel disease.

In addition, due to lymphoblast expression of TRAIL receptor polypeptides, antibodies and antibody compositions of the invention may be used to treat this form of cancer. Further, antibodies and antibody compositions of the invention may be used to treat various chronic and acute forms of inflammation such as rheumatoid arthritis, osteoarthritis, psoriasis, septicemia, and inflammatory bowel disease.

In one embodiment, antibodies and antibody compositions of the invention may be used to treat cardiovascular disorders, including peripheral artery disease, such as limb ischemia.

Cardiovascular disorders include cardiovascular abnormalities, such as arterio-arterial fistula, arteriovenous fistula, cerebral arteriovenous malformations, congenital heart defects, pulmonary atresia, and Scimitar Syndrome. Congenital heart defects include aortic coarctation, cor triatriatum, coronary vessel anomalies, crisscross heart, dextrocardia, patent ductus arteriosus, Ebstein's anomaly, Eisenmenger complex, hypoplastic left heart syndrome, levocardia, tetralogy of fallot, transposition of great vessels, double outlet right ventricle, tricuspid atresia, persistent truncus arteriosus, and heart septal defects, such as aortopulmonary septal defect, endocardial cushion defects, Lutembacher's Syndrome, trilogy of Fallot, ventricular heart septal defects.

Cardiovascular disorders also include heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, Scimitar Syndrome, cardiovascular syphilis, and cardiovascular tuberculosis.

Arrhythmias include sinus arrhythmia, atrial fibrillation, atrial flutter, bradycardia, extrasystole, Adams-Stokes Syndrome, bundle-branch block, sinoatrial block, long QT syndrome, parasystole, Lown-Ganong-Levine Syndrome, Mahaim-type pre-excitation syndrome, Wolff-Parkinson-White syndrome, sick sinus syndrome, tachycardias, and ventricular fibrillation. Tachycardias include paroxysmal tachycardia, supraventricular tachycardia, accelerated idioventricular rhythm, atrioventricular nodal reentry tachycardia, ectopic atrial tachycardia, ectopic junctional tachycardia, sinoatrial nodal reentry tachycardia, sinus tachycardia, Torsades de Pointes, and ventricular tachycardia.

Heart valve disease include aortic valve insufficiency, aortic valve stenosis, hear murmurs, aortic valve prolapse, mitral valve prolapse, tricuspid valve prolapse, mitral valve insufficiency, mitral valve stenosis, pulmonary atresia, pulmonary valve insufficiency, pulmonary valve stenosis, tricuspid atresia, tricuspid valve insufficiency, and tricuspid valve stenosis.

Myocardial diseases include alcoholic cardiomyopathy, congestive cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Kearns Syndrome, myocardial reperfusion injury, and myocarditis.

Myocardial ischemias include coronary disease, such as angina pectoris, coronary aneurysm, coronary arteriosclerosis, coronary thrombosis, coronary vasospasm, myocardial infarction and myocardial stunning.

Cardiovascular diseases also include vascular diseases such as aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leniche's Syndrome, arterial occlusive diseases, arteritis, enarteritis, polyarteritis nodosa, cerebrovascular disorders, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

Aneurysms include dissecting aneurysms, false aneurysms, infected aneurysms, ruptured aneurysms, aortic aneurysms, cerebral aneurysms, coronary aneurysms, heart aneurysms, and iliac aneurysms.

Arterial occlusive diseases include arteriosclerosis, intermittent claudication, carotid stenosis, fibromuscular dysplasias, mesenteric vascular occlusion, Moyamoya disease, renal artery obstruction, retinal artery occlusion, and thromboangiitis obliterans.

Cerebrovascular disorders include carotid artery diseases, cerebral amyloid angiopathy, cerebral aneurysm, cerebral anoxia, cerebral arteriosclerosis, cerebral arteriovenous malformation, cerebral artery diseases, cerebral embolism and thrombosis, carotid artery thrombosis, sinus thrombosis, Wallenberg's syndrome, cerebral hemorrhage, epidural hematoma, subdural hematoma, subaraxhnoid hemorrhage, cerebral infarction, cerebral ischemia (including transient), subclavian steal syndrome, periventricular leukomalacia, vascular headache, cluster headache, migraine, and vertebrobasilar insufficiency.

Embolisms include air embolisms, amniotic fluid embolisms, cholesterol embolisms, blue toe syndrome, fat embolisms, pulmonary embolisms, and thromoboembolisms. Thrombosis include coronary thrombosis, hepatic vein thrombosis, retinal vein occlusion, carotid artery thrombosis, sinus thrombosis, Wallenberg's syndrome, and thrombophlebitis.

Ischemia includes cerebral ischemia, ischemic colitis, compartment syndromes, anterior compartment syndrome, myocardial ischemia, reperfusion injuries, and peripheral limb ischemia. Vasculitis includes aortitis, arteritis, Behcet's Syndrome, Churg-Strauss Syndrome, mucocutaneous lymph node syndrome, thromboangiitis obliterans, hypersensitivity vasculitis, Schoenlein-Henoch purpura, allergic cutaneous vasculitis, and Wegener's granulomatosis.

In one embodiment, antibodies and antibody compositions of the invention is used to treat thrombotic microangiopathies. One such disorder is thrombotic thrombocytopenic purpura (TTP) (Kwaan, H. C., Semin. Hematol. 24:71 (1987); Thompson et al., Blood 80:1890 (1992)). Increasing TTP-associated mortality rates have been reported by the U.S. Centers for Disease Control (Torok et al., Am. J. Hematol. 50:84 (1995)). Plasma from patients afflicted with TTP (including HIV+ and HIV− patients) induces apoptosis of human endothelial cells of dermal microvascular origin, but not large vessel origin (Laurence et al., Blood 87:3245 (1996)). Plasma of TTP patients thus is thought to contain one or more factors that directly or indirectly induce apoptosis. As described in International patent application number WO 97/01715 (hereby incorporated by reference), TRAIL is present in the serum of TTP patients, and is likely to play a role in inducing apoptosis of microvascular endothelial cells. Another thrombotic microangiopathy is hemolytic-uremic syndrome (HUS) (Moake, J. L., Lancet, 343:393 (1994); Melnyk et al., (Arch. Intern. Med., 155:2077 (1995); Thompson et al., supra). Thus, in one embodiment, the invention is directed to use of antibodies and antibody compositions of the invention to treat the condition that is often referred to as “adult HUS” (even though it can strike children as well). A disorder known as childhood/diarrhea-associated HUS differs in etiology from adult HUS. In another embodiment, conditions characterized by clotting of small blood vessels may be treated using of antibodies and antibody compositions of the invention. Such conditions include, but are not limited to, those described herein. For example, cardiac problems seen in about 5-10% of pediatric AIDS patients are believed to involve clotting of small blood vessels. Breakdown of the microvasculature in the heart has been reported in multiple sclerosis patients. As a further example, treatment of systemic lupus erythematosus (SLE) is contemplated. In one embodiment, antibodies and antibody compositions of the invention, preferably antagonistic anti-TRAIL receptor antibodies of the invention, may be administered in vivo to a patient afflicted with a thrombotic microangiopathy. Thus, the present invention provides a method for treating a thrombotic microangiopathy, involving use of an effective amount of an antibody or antibody composition of the invention.

Antibodies and antibody compositions of the invention may be employed in combination with other agents useful in treating a particular disorder. For example, in an in vitro study reported by Laurence et al. (Blood 87:3245 (1996)), some reduction of TTP plasma-mediated apoptosis of microvascular endothelial cells was achieved by using an anti-Fas blocking antibody, aurintricarboxylic acid, or normal plasma depleted of cryoprecipitate. Thus, a patient may be treated with an antibody or antibody composition of the invention in combination with an agent that inhibits Fas-ligand-mediated apoptosis of endothelial cells, such as, for example, an agent described above. In one embodiment, antibodies of the invention and an anti-FAS blocking antibody are both administered to a patient afflicted with a disorder characterized by thrombotic microanglopathy, such as TTP or HUS. Examples of blocking monoclonal antibodies directed against Fas antigen (CD95) are described in International patent application publication number WO 95/10540, hereby incorporated by reference.

The naturally occurring balance between endogenous stimulators and inhibitors of angiogenesis is one in which inhibitory influences predominate (Rastinejad et al., Cell 56:345-355 (1989)). In those rare instances in which neovascularization occurs under normal physiological conditions, such as wound healing, organ regeneration, embryonic development, and female reproductive processes, angiogenesis is stringently regulated and spatially and temporally delimited. Under conditions of pathological angiogenesis such as that characterizing solid tumor growth, these regulatory controls fail. Unregulated angiogenesis becomes pathologic and sustains progression of many neoplastic and non-neoplastic diseases. A number of serious diseases are dominated by abnormal neovascularization including solid tumor growth and metastases, arthritis, some types of eye disorders, and psoriasis. See, e.g., reviews by Moses et al., Biotech. 9:710-714 (1991); Folkman et al., N. Engl. J. Med., 353:1757-1771 (1995); Auerbach et al., J. Microvasc. Res. 29:401-411 (1985); Folkman, Advances in Cancer Research, eds. Klein and Weinhouse, Academic Press, New York, pp. 175-203 (1985); Patz, Am. J. Opthalmol. 94:715-743 (1982); and Folkman et al., Science 221:719-725 (1983). In a number of pathological conditions, the process of angiogenesis contributes to the disease state. For example, significant data have accumulated which suggest that the growth of solid tumors is dependent on angiogenesis. Folkman and Klagsbrun, Science 235:442-447 (1987).

The present invention provides for treatment of diseases or disorders associated with neovascularization by administration of an antibody or antibody compositions of the invention. Malignant and metastatic conditions which can be treated with the polynucleotides and polypeptides of the invention include, but are not limited to those malignancies, solid tumors, and cancers described herein and otherwise known in the art (for a review of such disorders, see Fishman et al., Medicine, 2d Ed., J. B. Lippincott Co., Philadelphia (1985)).

Additionally, ocular disorders associated with neovascularization which can be treated with an antibody or antibody composition of the invention include, but are not limited to: neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization. See, e.g., reviews by Waltman et al., Am. J. Ophthal. 85:704-710 (1978) and Gartner et al., Surv. Ophthal. 22:291-312 (1978).

Additionally, disorders which can be treated with an antibody or antibody composition of the invention include, but are not limited to, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderma, trachoma, and vascular adhesions.

Antibodies and antibody compositions of the invention are useful in the diagnosis and treatment or prevention of a wide range of diseases and/or conditions. Such diseases and conditions include, but are not limited to, cancer (e.g., immune cell related cancers, breast cancer, prostate cancer, ovarian cancer, follicular lymphoma, cancer associated with mutation or alteration of p53, brain tumor, bladder cancer, uterocervical cancer, colon cancer, colorectal cancer, non-small cell carcinoma of the lung, small cell carcinoma of the lung, stomach cancer, etc.), lymphoproliferative disorders (e.g., lymphadenopathy), microbial (e.g., viral, bacterial, etc.) infection (e.g., HIV-1 infection, HIV-2 infection, herpesvirus infection (including, but not limited to, HSV-1, HSV-2, CMV, VZV, HHV-6, HHV-7, EBV), adenovirus infection, poxvirus infection, human papilloma virus infection, hepatitis infection (e.g., HAV, HBV, HCV, etc.), Helicobacter pylori infection, invasive Staphylococcia, etc.), parasitic infection, nephritis, bone disease (e.g., osteoporosis), atherosclerosis, pain, cardiovascular disorders (e.g., neovascularization, hypovascularization or reduced circulation (e.g., ischemic disease (e.g., myocardial infarction, stroke, etc.))), AIDS, allergy, inflammation, neurodegenerative disease (e.g., Alzheimer's disease, Parkinson's disease, amyotrophic lateral sclerosis, pigmentary retinitis, cerebellar degeneration, etc.), graft rejection (acute and chronic), graft vs. host disease, diseases due to osteomyclodysplasia (e.g., aplastic anemia, etc.), joint tissue destruction in rheumatism, liver disease (e.g., acute and chronic hepatitis, liver injury, and cirrhosis), autoimmune disease (e.g., multiple sclerosis, rheumatoid arthritis, systemic lupus erythematosus, autoimmune lymphoproliferative syndrome (ALPS), immune complex glomerulonephritis, autoimmune diabetes, autoimmune thrombocytopenic purpura, Grave's disease, Hashimoto's thyroiditis, etc.), cardiomyopathy (e.g., dilated cardiomyopathy), diabetes, diabetic complications (e.g., diabetic nephropathy, diabetic neuropathy, diabetic retinopathy), influenza, asthma, psoriasis, glomerulonephritis, septic shock, and ulcerative colitis.

Antibodies and antibody compositions of the invention are useful in promoting angiogenesis, wound healing (e.g., wounds, burns, and bone fractures).

Antibodies and antibody compositions of the invention are also useful as an adjuvant to enhance immune responsiveness to specific antigen, such as in anti-viral immune responses.

More generally, antibodies and antibody compositions of the invention are useful in regulating (i.e., elevating or reducing) immune response. For example, antibodies and antibody compositions of the invention may be useful in preparation or recovery from surgery, trauma, radiation therapy, chemotherapy, and transplantation, or may be used to boost immune response and/or recovery in the elderly and immunocompromised individuals. Alternatively, antibodies and antibody compositions of the invention are useful as immunosuppressive agents, for example in the treatment or prevention of autoimmune disorders. In specific embodiments, antibodies and antibody compositions of the invention are used to treat or prevent chronic inflammatory, allergic or autoimmune conditions, such as those described herein or are otherwise known in the art.

Therapeutic/Prophylactic Compositions and Administration

The invention provides methods of treatment, inhibition and prophylaxis by administration to a subject of an effective amount of antibody (or fragment or variant thereof) or pharmaceutical composition of the invention, preferably an antibody of the invention. In a preferred aspect, an antibody or fragment or variant thereof is substantially purified (i.e., substantially free from substances that limit its effect or produce undesired side-effects). The subject is preferably an animal, including but not limited to, animals such as cows, pigs, horses, chickens, cats, dogs, etc., and is preferably a mammal, and most preferably a human.

Formulations and methods of administration that can be employed when the compound comprises a nucleic acid or an immunoglobulin are described above; additional appropriate formulations and routes of administration can be selected from among those described herein below.

Various delivery systems are known and can be used to administer antibody or fragment or variant thereof of the invention, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of introduction include, but are not limited to, intradermal, intramuscular, intraperitoneal, intravenous, subcutaneous, intranasal, intracerebral, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the composition can be delivered in a vesicle, in particular a liposome (see Langer, Science 249:1527-1535 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the composition can be delivered in a controlled release system. In one embodiment, a pump may be used (see Langer, supra; Sefton, CRC Crit. Ref. Biomed. Eng. 14:20 1 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:71 (1983); see also Levy et al., Science 228:190 (1985); During et al., Ann. Neurol. 25:35 1 (1989); Howard et al., J. Neurosurg. 7 1:105 (1989)). In yet another embodiment a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1535 (1990)).

In a specific embodiment where the composition of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of an antibody or a fragment thereof, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the antibody or fragment thereof, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the composition of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg (e.g., 3 mg/kg or 5 mg/kg) of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of therapeutic or pharmaceutical compositions of the invention may be reduced by enhancing uptake and tissue penetration (e.g., into the brain) of the antibodies by modifications such as, for example, lipidation.

Antibodies of the invention may be formulated in pharmaceutically acceptable carriers. A formulation of an antibody of the invention may comprise a buffer. Buffers are well-known in the art and may be routinely applied to maintain the desired pH of the solution compositions of the invention. Suitable buffers for use in the preparation of a pharmaceutical composition of the invention include, for example, those described below.

Suitable buffers for use in the preparation of a antibody composition of the invention may include, but are not limited to, citrate, acetate, phosphate, carbonate, diphosphate, glycyl-glycine-piperazine-2HCl-NaOH; MES-NaOH-NaCl; TRIS-malic acid-NaOH; MES-NaOH; ACES-NaOH-NaCl; BES-NaOH-NaCl; MOPS-NaOH-NaCl; TES-NaOH-NaCl; MOPS-KOH; HEPES-NaOH-NaCl; TRIS-HCl; HEPPSO-NaOH; TAPS-NaOH-NaCl; HEPPS (EPPS)-NaOH; citric acid-disodiumhydrogenphosphate; boric acid-citric acid-potassium dihydrogen phosphate-Diethyl-barbituric acid-NaOH; citric acid-sodium citrate; sodium acetate-acetic acid; histidine; phosphate; potassium hydrogenphthalate-NaOH; cacodylic acid sodium salt-HCl; potassium dihydrogen phosphate-disodium hydrogenphosphate; potassium dihydrogen-phosphate-NaOH; sodium dihydrogen phosphate-disodium hydrogen phosphate; imidazole-HCl; sodium tetraborate-boric acid; 2-amino-2-methyl-1,3-propanediol-HCl; diethanolamine-HCl; potassium chloride-boric acid-NaOH; boric acid-NaOH-KCl; glycine-NaOH; and sodium carbonate-sodium hydrogen carbonate.

In one embodiment, the buffer is a citrate buffer or an acetate buffer. In another embodiment, the buffer includes an acetate buffer having a concentration of about 1 to about 50 mM and having a NaCl concentration of about 1 to about 500 mM. In another embodiment, the buffer includes an acetate buffer having a concentration of about 10 mM and having a NaCl concentration of about 140 mM. Suitable acetate buffers include acetate buffers having a concentration of about 1, 20, 25, 50, 75, 100, 200, 250, 300, 400, or 500 mM. Suitable buffers and solutions include those having a NaCl concentration of about 1, 50, 75, 100, 125, 140, 150, 175, 200, 225, 250, 275, 300, 350, 400, 450, or 500 mM. An additional suitable buffer is a HEPES buffer, in particular a HEPES buffer having a concentration of about 10, 20, 30, 40, 50, 60, 70, 80, 90, or 100 mM. In an additional embodiment, the solution comprises a HEPES buffer having a concentration of about 50 mM.

In other embodiments, antibodies of the invention are formulated in a citrate buffered solution that has a pH in the range of 5.5 to 6.5. In further embodiments, antibodies of the invention are formulated in a citrate buffered solution that has a pH of approximately or exactly 6.0. In other embodiments, antibodies of the invention are formulated in a citrate buffered solution that has a pH in the range of 5.5 to 6.5 and which also contains between 0 and 2.0%, preferably between 0 and 0.1% and more preferably less than 0.05%, of a surfactant such as Tween 80 or polysorbate 80.

In one embodiment, antibodies of the invention are formulated in 10 mM sodium citrate, 1.9% glycine, 0.5% sucrose, 0.02% polysorbate 80, pH 6.5.

In other embodiments, antibodies of the invention are formulated in a histidine buffered solution that has a pH in the range of 6.5 to 7.5. In other embodiments, antibodies of the invention are formulated in a histidine buffered solution that has a pH in the range of 6.5 to 7.5 and which also contains between 0 and 2.0%, preferably between 0 and 0.1% and more preferably less than 0.05%, of a surfactant such as Tween 80 or polysorbate 80.

In other embodiments, antibodies of the invention are formulated in a phosphate buffered solution that has a pH in the range of 7.0 to 8.0. In other embodiments, antibodies of the invention are formulated in a phosphate buffered solution that has a pH in the range of 7.0 to 8.0 and which also contains between 0 and 2.0%, preferably between 0 and 0.1% and more preferably less than 0.05%, of a surfactant such as Tween 80 or polysorbate 80.

Generally, administration of products of a species origin or species reactivity (in the case of antibodies) that is the same species as that of the patient is preferred. Thus, in a preferred embodiment, human antibodies, fragments, or variants, (e.g., derivatives), or nucleic acids, are administered to a human patient for therapy or prophylaxis.

It is preferred to use high affinity and/or potent in vivo inhibiting and/or neutralizing antibodies of the invention (including molecules comprising, or alternatively consisting of, antibody fragments or variants thereof) that immunospecifically bind to one or more TRAIL receptors, or polynucleotides encoding antibodies that immunospecifically bind to one or more TRAIL receptors, for both immunoassays and therapy of disorders related to TRAIL receptor polynucleotides or polypeptides, including fragments thereof. Such antibodies will preferably have an affinity for TRAIL receptor polypeptides and/or TRAIL receptor polypeptide fragments. Preferred binding affinities include those with a dissociation constant or K_{D} less than 5×10⁻² M, 10⁻² M, 5×10⁻³ M, 10⁻³ M, 5×10⁻⁴ M, 10⁻⁴ M, 5×10⁻⁵ M, 10⁻⁴ M, 5×10⁻⁶ M, 10⁻⁶ M, 5×10−7 M, 10⁻⁻⁷ M, 5×10⁻⁸ M, or 10⁻⁸ M. Even more preferred binding affinities include those with a dissociation constant or K_{D} less than 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. In a preferred embodiment, antibodies of the invention induce apoptosis of TRAIL receptor expressing cells.

As discussed in more detail below, the antibodies of the present invention may be used either alone or in combination with other compositions. The antibodies may further be recombinantly fused to a heterologous polypeptide at the N- or C-terminus or chemically conjugated (including covalent and non-covalent conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

The antibody and antibody compositions of the invention may be administered alone or in combination with other therapeutic agents, including but not limited to chemotherapeutic agents, antibiotics, antivirals, anti-retroviral agents, steroidal and non-steroidal anti-inflammatories, conventional immunotherapeutic agents and cytokines. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In preferred embodiments, antibodies of the invention that are administered to an animal, preferably a human, for therapeutic uses are multimeric antibodies. In specific embodiments, antibodies of the invention are homodimeric IgG molecules. In other specific embodiments, antibodies of the invention are homodimeric IgG1 molecules. In specific embodiments, antibodies of the invention are homotrimeric IgG molecules. In other specific embodiments, antibodies of the invention are trimeric IgG1 molecules. In other specific embodiments, antibodies of the invention are higher-order multimers of IgG molecules (e.g., tetramers, penatmers and hexamers]. In still further specific embodiments, antibodies of the IgG molecules comprising the higher order multimers of IgG molecules are IgG1 molecules.

Alternatively, antibodies of the invention for therapeutic uses may be administered in combination with crosslinking agents known in the art, including but not limited to, anti-IgG antibodies.

Combination Therapies with Anti-TR4 Antibodies, TRAIL, Apoptosis Inducing Peptides and/or Chemotherapeutic Agents

Anti-TRAIL Receptor antibodies may be administered in combination with other anti-TRAIL Receptor antibodies, TRAIL, and/or chemotherapeutics.

In specific embodiments, an antibody of the invention that specifically binds TR4 is used or administered in combination with a second antibody that specifically binds TR7. In another embodiment, the antibodies specific for TR4 and TR7 are agonistic antibodies that induce apoptosis of TRAIL receptor expressing cells (e.g. cells that express TR4 and TR7). In a specific embodiment, the combination of anti-TR4 treatment and anti-TR7 treatment induces more apoptosis of TRAIL receptor expressing cells than either anti-TR4 antibody treatment or anti-TR7 antibody treatment alone. The anti-TR4 and anti-TR7 antibodies can be administered either simultaneously, sequentially, or a combination of simultaneous or sequential administration throughout the dosage regimen. In another specific embodiment anti-TR4 and anti-TR7 antibodies are used or administered in combination with a chemotherapeutic drug, such as those described herein (see, for example, below and Example 4). In a particular embodiment, the synergistic induction of apoptosis resulting from anti-TR4 and anti-TR7 antibody treatment, is more evident or more pronounced when the anti-TR4 and anti-TR7 antibodies are used or administered in combination with a chemotherapeutic agent and/or a cross-linking reagent.

In other embodiments, anti-TR4 antibody, anti-TR7 antibody, or both anti-TR4 and anti-TR7 antibodies may be administered in combination with anti-TR5 and/or anti-TR10 antibodies. In preferred embodiments, the anti-TR5 and/or anti-TR10 antibodies inhibit the binding of TRAIL to TR5 and TR10, respectively.

In a specific embodiment, antibodies or antibody compositions of the invention are administered in combination with DAB₃₈₉EGF, a diphtheria toxin fused to Epidermal Growth Factor. DAB₃₈₉EGF is described in Shaw et al., (1991) The Journal of Biological Chemistry, 266:21118-24, which is hereby incorporated by reference in its entirety. In a specific embodiment, antibodies or antibody compositions of the invention are administered in combination with DAB₃₈₉EGF for the treatment of cancer, such as brain cancers and epithelial cancers. In a specific embodiment, antibodies or antibody compositions of the invention are administered in combination with DAB₃₈₉EGF for the treatment of astrocytomas. In a specific embodiment, antibodies or antibody compositions of the invention are administered in combination with DAB₃₈₉EGF for the treatment of glioblastyoma multiforme (GBM).

In a preferred embodiment, compositions of the invention are administered in combination with a chemotherapeutic agent. Chemotherapeutic agents that may be administered with the compositions of the invention include, but are not limited to, antibiotic derivatives (e.g., doxorubicin (adriamycin), bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, etoposide, Topotecan, 5-Fluorouracil, paclitaxel (Taxol), Cisplatin, Cytarabine, and IFN-gamma, irinotecan (Camptosar, CPT-11), gemcitabine ((GEMZAR™), and oxaliplatin, ifosamide, nitrosourea compounds).

Therapeutic agents, useful in the treatment, prevention, amelioration and/or cure of cancers, with which antibodies of the present invention may be administered, include, for example, biological agents (e.g., inhibitors of signaling pathways, inhibitors of gene transcription, inhibitors of multi-drug resistance (MDR) mechanisms, inhibitors of angiogenesis, inhibitors of matrix metalloproteinases, proteasome inhibitors, hormones and hormone antagonists, and compounds of unknown mechanism), chemotherapeutic agents (e.g., alkylating agents, antimetabolites, farnesyl transferase inhibitors, mitotic spindle inhibitors (plant-derived alkaloids), nucleotide analogs, platinum analogs, and topoisomerase inhibitors), corticosteroids, gene therapies, immunotherapeutic agents (e.g., monoclonal antibodies, cytokines and vaccines), phototherapy, radiosensitizing agents, treatment support agents (e.g., anti-emetic agents, analgesic agents and hematopoietic agents), and other miscellaneous drug types. Therapeutic procedures, useful in the treatment, prevention, amelioration and/or cure of cancers, with which agonistic antibodies of the present invention may be administered, include, for example, but are not limited to, surgical procedures and radiation therapies.

In preferred embodiments, agonistic antibodies of the invention are administered in combination with one or more of the above-described therapeutic agents in the treatment, prevention, amelioration and/or cure of cancers and premalignant conditions.

In specific embodiments, antibodies of the present invention may be administered in combination with one or more chemotherapeutic or other therapeutic agents useful in the treatment, prevention, amelioration and/or cure of cancers including, but not limited to, 81C6 (Anti-tenascin monoclonal antibody), 2-chlorodeoxyadenosine, A007 (44′-dihydroxybenzophenone-2,4-dinitrophenylhydrazone), Abarelix® (Abarelix-Depot-M®, PPI-149, R-3827); Abiraterone acetate® (CB-7598, CB-7630), ABT-627 (ET-1 inhibitor), ABX-EGF (anti-EGFr MAb), Acetyldinaline (CI-994, GOE-5549, GOR-5549, PD-130636), AG-2034 (AG-2024, AG-2032, GARFT [glycinamide ribonucleoside transformylase] inhibitor), Alanosine, Aldesleukin (IL-2, Proleukin®), Alemtuzumab® (Campath®), Alitretinoin (Panretin®, LGN-1057), Allopurinol (Aloprim®, Zyloprim®), Altretamine (Hexalen®, hexamethylmelamine, Hexastat®), Amifostine (Ethyol®), Aminocamptothecin (9-AC, 9-Aminocamptothecin, NSC 603071), Aminoglutethimide (Cytadren®), Aminolevulinic acid (Levulan®, Kerastick®), Aminopterin, Amsacrine, Anastrozole (Arimidex®), Angiostatin, Annamycin (AR-522, annamycin LF, Aronex®), Anti-idiotype therapy (BsAb), Anti-CD19/CD3 MAb (anti-CD19/CD3 scFv, anti-NHL MAb), APC-8015 (Provenge®, Dendritic cell therapy), Aplidine (Aplidin®, Aplidina®), Arabinosylguanine (Ara-G, GW506U78, Nelzarabineg, Compound 506U78), Arsenic trioxide (Trisenox®, ATO, Atrivex®), Avorelin® (Meterelin®, MF-6001, EP-23904), B43-Genistein (anti-CD19 Ab/genistein conjugate), B43-PAP (anti-CD19 Ab/pokeweed antiviral protein conjugate), B7 antibody conjugates, BAY 43-9006 (Raf kinase inhibitor), BBR 3464, Betathine (Beta-LT), Bevacizumab® (Anti-VEGF monoclonal antibody, rhuMAb-VEGF), Bexarotene (Targretin®, LGD1069), BIBH-1 (Anti-FAP MAb), BIBX-1382, Biclutamide (Casodex®), Biricodar dicitrate (Incel®, Incel MDR Inhibitor), Bleomycin (Blenoxane®), BLP-25 (MUC-1 peptide), BLyS antagonists, BMS-214662 (BMS-192331, BMS-193269, BMS-206635), BNP-1350 (BNPI-1100, Karenitecins), Boronated Protoporphyrin Compound (PDIT, Photodynamic Immunotherapy), Bryostatin-1 (Bryostatin®, BMY45618, NSC-339555), Budesonide (Rhinocort®), Busulfan (Busulfex®, Myleran®), C225 (IMC-225, EGFR inhibitor, Anti-EGFr MAb, Cetuximab®), C242-DMI (huC242-DM1), Cabergoline (Dostinex®), Capecitabine (Xeloda®, Doxifluridine®, oral 5-FU), Carbendazin® (FB-642), Carboplatin (Paraplatin®, CBDCA), Carboxyamidotriazole (NSC 609974, CAI, L-651582), Carmustine (DTI-015, BCNU, BiCNU, Gliadel Wafer®), CC49-zeta gene therapy, CEA-cide® (Labetuzumab®, Anti-CEA monoclonal antibody, hMN-14), CeaVac® (MAb 3H1), Celecoxib (Celebrex®), CEP-701 (KT-5555), Cereport® (Lobradimil®, RMP-7), Chlorambucil (Leukeran®), CHML (Cytotropic Heterogeneous Molecular Lipids), Cholecaliferol, CI-1033 (Pan-erbB RTK inhibitor), Cilengitide (EMD-121974, integrin alphavbeta3 antagonist), Cisplatin (Platinole, CDDP), Cisplatin-epinephrine gel (IntraDose®, FocaCist®), Cisplatin-liposomal (SPI-077), 9-cis retinoic acid (9-cRA), Cladribine (2-CdA, Leustatin®), Clofarabine (chloro-fluoro-araA), Clonadine hydrochloride (Duraclon®), CMB401 (Anti-PEM MAb/calicheamycin), CMT-3 (COL-3, Metastat®), Cordycepin, Cotara® (chTNT-1/B, [¹³¹I]-chTNT-1/B), CN-706, CP-358774 (Tarceva®, OSI-774, EGFR inhibitor), CP-609754, CP IL-4-toxin (IL-4 fusion toxin), CS-682, CT-2584 (Apra®, CT-2583, CT-2586, CT-3536), CTP-37 (Avicine®, hCG blocking vaccine), Cyclophosphamide (Cytoxan®, Neosar®, CTX), Cytarabine (Cytosar-U®, ara-C, cytosine arabinoside, DepoCyt®), D-limonene, DAB389-EGF (EGF fusion toxin), Dacarbazine (DTIC), Daclizumab® (Zenapax®), Dactinomycin (Cosmegen®), Daunomycin (Daunorubicin®, Cerubidine®), Daunorubicin (DaunoXomeg, Daunorubicin®, Cerubidine®), DeaVac® (CEA anti-idiotype vaccine), Decitabine (5-aza-2′-deoxyytidine), Declopramide (Oxi-104), Denileukin diftitox (Ontak®), Depsipeptide (FR901228, FK228), Dexamethasone (Decadron®), Dexrazoxane (Zinecarde), Diethylnorspermine (DENSPM), Diethylstilbestrol (DES), Dihydro-5-azacytidine, Docetaxel (Taxotere®, Taxane®), Dolasetron mesylate (Anzemet®)), Dolastatin-10 (DOLA-10, NSC-376128), Doxorubicin (Adriamycin®, Doxil®, Rubex®), DPPE, DX-8951f (DX-8951), Edatrexate, EGF-P64k Vaccine, Elliott's B Solution®, EMD-121974, Endostatin, Eniluracil (776c85), EO9 (EO1, EO4, EO68, EO70, EO72), Epirubicin (Ellence®, EPI, 4′ epi-doxorubicin), Epratuzumab® (Lymphocide®, humanized anti-CD22, HAT), Erythropoietin (EPO®, Epogen®, Procrit®), Estramustine (Emcyt®), Etanidazole (Radinyl®), Etoposide phosphate (Etopophos®), Etoposide (VP-16, Vepesid®), Exemestane (Aromasin®, Nikidess®), Exetecan mesylate (DX-8951, DX-8951f), Exisulind (SAAND, Aptosyn®), cGMP-PDE2 and 5 inhibitor), F19 (Anti-FAP monoclonal antibody, iodinated anti-FAP MAb), Fadrozole (Afema®, Fadrozole hydrochloride, Arensin®), Fenretinide® (4HPR), Fentanyl citrate (Actiq®), Filgrastim (Neupogen®, G-CSF), FK-317 (FR-157471, FR-70496), Flavopiridol (HMR-1275), Fly3/flk2 ligand (Mobista®), Fluasterone, Fludarabine (Fludara®, FAMP), Fludeoxyglucose (F-18®), Fluorouracil (5-FU, Adrucil®, Fluoroplex®, Efudex®), Flutamide (Eulexin®), FMdC (KW-2331, MDL-101731), Fommestane (Lentaron®), Fotemustine (Muphoran®, Mustophoran®), FUDR (Floxundine®), Fulvestrant (Faslodex®), G3139 (Genasense®, GentaAnticode®, Bcl-2 antisense), Gadolinium texaphyrin (Motexafin gadolinium, Gd-Tex®, Xcytrin®), Galarubicin hydrochloride (DA-125), GBC-590, Gastrimmune® (Anti-gastrin-17 immunogen, anti-g17), Gemcitabine (Gemto®, Gemzar®), Gentuzumab-ozogamicin (Mylotarg®), GL331, Globo H hexasaccharide (Globo H-KLH®), Glufosfamide® (β-D-glucosyl-isofosfamide mustard, D19575, INN), Goserelin acetate (Zoladex®), Granisetron (Kytril®), GVAX (GM-CSF gene therapy), Her-2/Neu vaccine, Herceptin® (Trastuzumab®, Anti-HER-2 monoclonal antibody, Anti-EGFR-2 MAb), HSPPC-96 (HSP cancer vaccine, gp96 heat shock protein-peptide complex), HuID10 (anti-HLA-DR MAb, SMART 1D10), HumaLYM (anti-CD20 MAb), Hydrocortisone, Hydroxyurea (Hydrea®), Hypencin (VIMRxyn®), I-131 Lipidiol®, Ibritumomab® tiuxetan (Zevalin®), Idarubicin (Idamycin®, DMDR, IDA), Ifosfamide (IFEXO), Imatinib mesylate (STI-571, Imatinib®, Glivec®, Gleevec®, Abl tyrosine kinase inhibitor), INGN-101 (p53 gene therapy/retrovirus), INGN-201 (p53 gene therapy/adenovirus), Interferon alpha (Alfaferone®, Alpha-IF®), Interferon alpha 2a (Intron A®), Interferon gamma (Gamma-interferon, Gamma 1000, Gamma-1F), Interleukin-2 (ProleiukinR®), Intoplicine (RP 60475), Irinotecan (Camptosar®, CPT-11, Topotecin®, CaptoCPT-1), Irofulven (MGI-114, Ivofulvan, Acylfulvene analogue), ISIS-2053 (PKC-alpha antisense), ISIS-2503 (Ras antisense), ISIS-3521 (PKC-alpha antisense), ISIS-5132 (K-ras/raf antisense), Isotretinoin (13-CRA, 13-cis retinoic acid, Accutane®), Ketoconazole (Nizoral®), KRN-8602 (MX, MY-5, NSC-619003, MX-2), L-778123 (Ras inhibitors), L-asparaginase (Elspar®, Crastinin®, Asparaginase medac®, Kidrolase®), Leflunomide (SU-101, SU-0200), Letrozole (Femara®), Leucovorin (Leucovorin®, Wellcovorin®), Leuprolide acetate (Viadur®, Lupron®, Leuprogel®, Eligard®), Leuvectin® (cytofectin+IL-2 gene, IL-2 gene therapy), Levamisole (Ergamisol®), Liarozole (Liazal, Liazol, R-75251, R-85246, Ro-85264), Lmb-2 immunotoxin (anti-CD25 recombinant immuno toxin, anti-Tac(Fv)-PE38), Lometrexol (T-64, T-904064), Lomustine (CCNU®, CeeNUM®), LY-335979, Lym-1 (131-1LYM-1), Lymphoma vaccine (Genitope), Mannan-MUC1 vaccine, Marimastat® (BB-2516, TA-2516, MMP inhibitor), MDX-447 (MDX-220, BAB-447, EMD-82633, H-447, anti-EGFr/FcGammaR1r), Mechlorethamine (Nitrogen Mustard, HN₂, Mustargen®), Megestrol acetate (Megace®, Pallace®), Melphalan (L-PAM, Alkeran®, Phenylalanine mustard), Mercaptopurine (6-mercaptopurine, 6-MP), Mesna (Mesnex®), Methotrexate® (MTTX Mexate®, Folex®), Methoxsalen (Uvadexe), 2-Methoxyestradiol (2-ME, 2-ME2), Methylprednisolone (Solumedrol®), Methyltestosterone (Android-10®, Testred®, Virilon®), MGV, Mitomycin C (Mitomycin®, Mutamycin®, Mito Extra®), Mitoxantrone (Novantrone®, DHAD), Mitumomab® (BEC-2, EMD-60205), Mivobulin isethionate (CI-980), MN-14 (Anti-CEA immunoradiotherapy, ¹³¹I-MN-14, ¹⁸⁸Re-MN-14), Motexafin Lutetium (Lutrin®, Optrin®, Lu-Tex®, lutetium texaphyrin, Lucyn®, Antrin®), MPV-2213ad (Finrozole®), MS-209, Muc-1 vaccine, NaPro Paclitaxel, Nelarabine (Compound 506, U78), Neovastat® (AE-941, MMP inhibitor), Neugene compounds (Oncomyc-NG, Resten-NG, myc antisense), Nilutamide (Nilandron®), NovoMAb-G2 scFv (NovoMAb-G2 IgM), O6-benzylguanine (BG, Procept®), Octreotide acetate (Sandostatin LAR® Depot), Odansetron (Zofran®), Onconase (Ranpimase®), OncoVAX-CL, OncoVAX-CL Jenner (GA-733-2 vaccine), OncoVAX-P (OncoVAX-PrPSA), Onyx-015 (p53 gene therapy), Oprelvekin (Neumage®), Orzel (Tegafur+Uracil+Leucovorin), Oxaliplatin (Eloxatinel, Eloxatin®), Pacis® (BCG, live), Paclitaxel (Paxene®, Taxol®), Paclitaxel-DHA (Taxoprexin®), Pamidronate (Aredia®), PC SPES, Pegademase (Adagen®, Pegademase bovine), Pegaspargase® (Oncospar®), Peldesine (BCX-34, PNP inhibitor), Pemetrexed disodium (Alimta®, MTA, multitargeted antifolate, LY 231514), Pentostatin (Nipente, 2-deoxycoformycin), Perfosfamide (4-hydroperoxycyclophosphamide, 4-HC), Perillyl alcohol (perilla alcohol, perillic alcohol, perillol, NSC-641066), Phenylbutyrate, Pirarubicin (THP), Pivaloyloxymethyl butyrate (AN-9, Pivanex®), Porfimer sodium (Photofrin®), Prednisone, Prinomastat® (AG-3340, MMP inhibitor), Procarbazine (Matulane®), PROSTVAC, Providence Portland Medical Center Breast Cancer Vaccine, PS-341 (LDP-341, 26S proteasome inhibitor), PSMA MAb (Prostate Specific Membrane Antigen monoclonal antibody), Pyrazoloacridine (NSC-366140, PD-115934), Quinine, R115777 (Zamestra®), Raloxifene hydrochloride (Evista®, Keoxifene hydrochloride), Raltitrexed (Tomudex®, ZD-1694), Rebeccamycin, Retinoic acid, R-flurbiprofen (Flurizan®, E-7869, MPC-7869), RFS-2000 (9-nitrocamptothecan, 9-NC, rubitecan®), Rituximab® (Rituxan®, anti-CD20 MAb), RSR-13 (GSJ-61), Satraplatin (BMS-182751, JM-216), SCH 6636, SCH-66336, Sizofilan® (SPG, Sizofuran®, Schizophyllan®, Sonifilan®), SKI-2053R (NSC-D644591), Sobuzoxane (MST-16, Perazolin®), Squalamine (MSI-1256F), SR49059 (vasopressin receptor inhibitor, Via), Streptozocin (Zanosar®), SU5416 (Semaxanib®, VEGF inhibitor), SU6668 (PDGF-TK inhibitor), T-67 (T-138067, T-607), Talc (Sclerosol®), Tamoxifen (Nolvadex®), Taurolidine (Taurolin®), Temozolamide (Temodar®, NSC 362856), Teniposide (VM-26, Vumon®), TER-286, Testosterone (Andro®, Androderm®, Testoderm TTS®, Testoderm®, Depo-Testosterone®, Androgel®, depoAndro®), Tf-CRM107 (Transferrin-CRM-107), Thalidomide and thalidomide analogs, including but not limited to, lenalidomide (CC-5013, REVLIMID) and CC4047 (ACTIMID), Theratope, Thioguanine (6-thioguanine, 6-TG), Thiotepa (triethylenethiophosphaoramide, Thioplex®), Thymosin alpha I (Zadaxin®, Thymalfasin®), Tiazofurin (Tiazole®), Tirapazamine (SR-259075, SR-4233, Tirazone®, Win-59075), TNP470 (AGM-1470, Fumagillin), Tocladesine (8-Cl-cAMP), Topotecan (Hycamtin®, SK&F-104864, NSC-609699, Evotopin®), Toremifene (Estrimex®, Fareston®), Tositumomab® (Bexxar®), Tretinoin (Retin-AS, Atragen®, ATRA, Vesanoid®), TriAb® (anti-idiotype antibody immune stimulator), Trilostane (Modrefen®), Triptorelin pamoate (Trelstar Depot®, Decapeptyl®), Trimetrexate (Neutrexin®), Troxacitabine (BCH-204, BCH-4556, Troxatyl®), TS-1, UCN-01 (7-hydroxystaurosporine), Valrubicin (Valstar®), Valspodar (PSC 833), Vapreotide® (BMY-41606), Vaxid (B-cell lymphoma DNA vaccine), Vinblastine (Velban®, VLB), Vincristine (Oncovin®, Onco TCS®, VCR, Leurocristine®), Vindesine (Eldisine®, Fildesin®), Vinflunine (Javlor®, tubulin polymerization inhibitor), Vinorelbine (Navelbine®), Vitaxin® (LM-609, integrin alphavbeta3 antagonistic MAb), WF10 (macrophage regulator), WHI-P131, WT1 Vaccine, XR-5000 (DACA), XR-9576 (XR-9351, P-glycoprotein/MDR inhibitor), ZD-9331, ZD-1839 (IRESSAS), and Zoledronate (Zometa®).

In preferred embodiments, agonistic antibodies of the invention are administered in combination with one or more of the above-described therapeutic agents in the treatment, prevention, amelioration and/or cure of cancers.

In one embodiment, antibodies of the present invention may be administered in combination with a taxane. In another embodiment, antibodies of the present invention may be administered in combination with a taxane for the treatment of cancers that are resistant to individual chemotherapies. In a specific embodiment, antibodies of the present invention may be administered in combination with Docetaxel (Taxotere®). In a specific embodiment, antibodies of the present invention may be administered in combination with Docetaxel (Taxotere®) for the treatment of cancers that are resistant to individual chemotherapies.

In one embodiment, agonistic antibodies of the present invention may be administered in combination with a taxane. In another embodiment, agonistic antibodies of the present invention may be administered in combination with a taxane for the treatment of cancers that are resistant to individual chemotherapies. In a specific embodiment, agonistic antibodies of the present invention may be administered in combination with Docetaxel (Taxotere®). In a specific embodiment, agonistic antibodies of the present invention may be administered in combination with Docetaxel (Taxotere®) for the treatment of cancers that are resistant to individual chemotherapies.

In one embodiment, antibodies of the present invention may be administered in combination with a platinum-based chemotherapeutic. In another embodiment, antibodies of the present invention may be administered in combination with a platinum-based chemotherapeutic for the treatment of cancers that are resistant to individual chemotherapies. In another specific embodiment, antibodies of the invention may be administered in combination with Carboplatin (Paraplatin®, CBDCA). In another specific embodiment, antibodies of the present invention may be administered in combination with Carboplatin (Paraplatin®, CBDCA) for the treatment of cancers that are resistant to individual chemotherapies.

In one embodiment, agonistic antibodies of the present invention may be administered in combination with a platinum-based chemotherapeutic. In another embodiment, agonistic antibodies of the present invention may be administered in combination with a platinum-based chemotherapeutic for the treatment of cancers that are resistant to individual chemotherapies. In another specific embodiment, agonistic antibodies of the invention may be administered in combination with Carboplatin (Paraplatin®, CBDCA). In another specific embodiment, agonistic antibodies of the present invention may be administered in combination with Carboplatin (Paraplatin®, CBDCA) for the treatment of cancers that are resistant to individual chemotherapies.

In one embodiment, antibodies of the present invention may be administered in combination with a topoisomerase inhibitor. In another embodiment, antibodies of the present invention may be administered in combination with a topoisomerase inhibitor for the treatment of cancers that are resistant to individual chemotherapies. In a specific embodiment, antibodies of the present invention may be administered in combination with Irinotecan (Camptosar®, CPT-11, Topotecin®, CaptoCPT-1). In a specific embodiment, antibodies of the present invention may be administered in combination with Irinotecan (Camptosar®, CPT-11, Topotecin®, CaptoCPT-1) for the treatment of cancers that are resistant to individual chemotherapies.

In one embodiment, agonistic antibodies of the present invention may be administered in combination with a topoisomerase inhibitor. In another embodiment, agonistic antibodies of the present invention may be administered in combination with a topoisomerase inhibitor for the treatment of cancers that are resistant to individual chemotherapies. In a specific embodiment, agonistic antibodies of the present invention may be administered in combination with Irinotecan (Camptosar®, CPT-11, Topotecin®, CaptoCPT-1). In a specific embodiment, agonistic antibodies of the present invention may be administered in combination with Irinotecan (Camptosar®, CPT-11, Topotecin®, CaptoCPT-1) for the treatment of cancers that are resistant to individual chemotherapies.

In one embodiment, antibodies of the present invention may be administered in combination with a fluoropyrimidine. In another embodiment, antibodies of the present invention may be administered in combination with a fluoropyrimidine for the treatment of cancers that are resistant to individual chemotherapies. In another specific embodiment, antibodies of the invention may be administered in combination with Fluorouracil (5-FU, Adrucil®). In another specific embodiment, antibodies of the present invention may be administered in combination with Fluorouracil (5-FU, Adrucil®) for the treatment of cancers that are resistant to individual chemotherapies.

In one embodiment, agonistic antibodies of the present invention may be administered in combination with a fluoropyrimidine. In another embodiment, agonistic antibodies of the present invention may be administered in combination with a fluoropyrimidine for the treatment of cancers that are resistant to individual chemotherapies. In another specific embodiment, agonistic antibodies of the invention may be administered in combination with Fluorouracil (5-FU, Adrucil®). In another specific embodiment, agonistic antibodies of the present invention may be administered in combination with Fluorouracil (5-FU, Adrucil®) for the treatment of cancers that are resistant to individual chemotherapies.

In further specific embodiments, antibodies of the present invention may be administered in combination with one or more combinations of therapeutic agents useful in the treatment, prevention, amelioration and/or cure of cancers including, but not limited to, 9-aminocamptothecin+G-CSF, Adriamycin®+Blenoxane+Vinblastine+Dacarbazine (ABVD), BCNU (Carmustine)+Etoposide+Ara-C (Cytarabine)+Melphalen (BEAM), Bevacizumab®+Leucovorin, Bleomycin+Etoposide+Platinol® (Cisplatin) (BEP), Bleomycin+Etoposide+Adriamycin+Cyclophosphamide+Vincristine+Procarbazine+Prednisone (BEACOPP), Bryostatin+Vincristine, Busulfan+Melphalan, Carboplatin+Cereport®, Carboplatin+Cyclophosphamide, Carboplatin+Paclitaxel, Carboplatin+Etoposide+Bleomycin (CEB), Carboplatin+Etoposide+Thiotepa, Cisplatin+Cyclophosphamide, Cisplatin+Docetaxel, Cisplatin+Doxorubicin, Cisplatin+Etoposide, Cisplatin+Gemcitabine, Cisplatin+Interferon alpha, Cisplatin+Irinotecan, Cisplatin+Paclitaxel, Cisplatin+Teniposide, Cisplatin+Vinblastine, Cisplatin+Vindesine, Cisplatin+Vinorelbine, Cisplatin+Cytarabine+Ifosfamide, Cisplatin+Ifosfamide+Vinblastine, Cisplatin+Vinblastine+Mitomycin C, Cisplatin+Vincristine+Fluorouracil, Cisplatin+Vincnistine+Lomustine, Cisplatin+Vinorelbine+Gemcitabine, Cisplatin+Carmustine+Dacarbazine+Tamoxifen, Cisplatin+Cyclophosphamide+Etoposide+Vincristine, Cisplatin (Platinol®)+Oncovin®+Doxorubicin (Adriamycin®)+Etoposide (CODE), Cisplatin+Cytarabine+Ifosfamide+Etoposide+Methotrexate, Cyclophosphamide+Adriamycin® (Doxorubicin), Cyclophosphamide+Melphalan, Cyclophosphamide+SCH 6636, Cyclophosphamide+Adriamycin®+Cisplatin (Platinol®) (CAP), Cyclophosphamide+Adriamycin®+Vincristine (CAV), Cyclophosphamide+Doxorubicin+Teniposide+Prednisone, Cyclophosphamide+Doxorubicin+Teniposide+Prednisone+Interferon alpha, Cyclophosphamide+Epirubicin+Cisplatin (Platinol®) (CEP), Cyclophosphamide+Epirubicin+Fluorouracil, Cyclophosphamide+Methotrexate+Fluoruracil (CMF), Cyclophosphamide+Methotrexate+Vincristine (CMV), Cyclophosphamide+Adriamycin®+Methotrexate+Fluorouracil (CAMF), Cyclophosphamide+Adriamycin®+Methotrexate+Procarbazine (CAMP), Cyclophosphamide+Adriamycin®+Vincristine+Etoposide (CAV-E), Cyclophosphamide+Adfiamycin®+Vincnistine+Prednisone (CHOP), Cyclophosphamide+Novantrone® (Mitoxantrone)+Vincristine (Oncovonin)+Prednisone (CNOP), Cyclophosphamide+Adriamycin®+Vincristine+Prednisone+Rituximab (CHOP+Rituximab), Cyclophosphamide+Adriamycin®+Vincristine+Teniposide (CAV-T), Cyclophosphamide+Adriamycin®+Vincristine alternating with Platinol®+Etoposide (CAV/PE), Cyclophosphamide+BCNU (Carmustine)+VP-16 (Etoposide) (CBV), Cyclophosphamide+Vincristine+Prednisone (CVP), Cyclophosphamide+Oncovin®+Methotrexate+Fluorouracil (COMF), Cytarabine+Methotrexate, Cytarabine+Bleomycin+Vincristine+Methotrexate (CytaBOM), Dactinomycin+Vincristine, Dexamethasone+Cytarabine+Cisplatin (DHAP), Dexamethasone+Ifosfamide+Cisplatin+Etoposide (DICE), Docetaxel+Gemcitabine, Docetaxel+Vinorelbine, Doxorubicin+Vinblastine+Mechlorethamine+Vincristine+Bleomycin+Etoposide+Prednisone (Stanford V), Epirubicin+Gemcitabine, Estramustine+Docetaxel, Estramustine+Navelbine, Estramustine+Paclitaxel, Estramustine+Vinblastine, Etoposide (Vepesid®)+Ifosfamide+Cisplatin (Platinol®) (VIP), Etoposide+Vinblastine+Adriamycin (EVA), Etoposide (Vepesid®)+Ifosfamide+Cisplatin+Epirubicin (VIC-E), Etoposide+Methylprednisone+Cytarabine+Cisplatin (ESHAP), Etoposide+Prednisone+Ifosfamide+Cisplatin (EPIC), Fludarabine+Mitoxantrone+Dexamethasone (FMD), Fludarabine+Dexamethasone+Cytarabine (ara-C)+Cisplatin (Platinol®) (FluDAP), Fluorouracil+Bevacizumab®, Fluorouracil+CeaVac®, Fluorouracil+Leucovorin, Fluorouracil+Levamisole, Fluorouracil+Oxaliplatin, Fluorouracil+Raltitrexed, Fluorouracil+SCH 6636, Fluorouracil+Trimetrexate, Fluorouracil+Leucovorin+Bevacizumab®, Fluorouracil+Leucovorin+Oxaliplatin, Fluorouracil+Leucovorin+Trimetrexate, Fluorouracil+Oncovin®+Mitomycin C (FOMi), Hydrazine+Adriamycin®+Methotrexate (HAM), Ifosfamide+Docetaxel, Ifosfamide+Etoposide, Ifosfamide+Gemcitabine, Ifosfamide+Paclitaxel, Ifosfamide+Vinorelbine, Ifosfamide+Carboplatin+Etoposide (ICE), Ifosfamide+Cisplatin+Doxorubicin, Irinotecan+C225 (Cetuximab®), Irinotecan+Docetaxel, Irinotecan+Etoposide, Irinotecan+Fluorouracil, Irinotecan+Gemcitabine, Mechlorethamine+Oncovin® (Vincristine)+Procarbazine (MOP), Mechlorethamine+Oncovin® (Vincristine)+Procarbazine+Prednisone (MOPP), Mesna+Ifosfamide+Idarubicin+Etoposide (MIZE), Methotrexate+Interferon alpha, Methotrexate+Vinblastine, Methotrexate+Cisplatin, Methotrexate with leucovorin rescue+Bleomycin+Adriamycin+Cyclophosphamide+Oncovorin+Dexamethasone (m-BACOD), Mitomycin C+Ifosfamide+Cisplatin (Platinol®) (MIP), Mitomycin C+Vinblastine+Paraplatin® (MVP), Mitoxantrone+Hydrocortisone, Mitoxantrone+Prednisone, Oncovin®+SCH 6636, Oxaliplatin+Leucovorin, Paclitaxel+Doxorubicin, Paclitaxel+SCH 6636, Paraplatin®+Docetaxel, Paraplatin®+Etoposide, Paraplatin®+Gemcitabine, Paraplatin®+Interferon alpha, Paraplatin®+Irinotecan, Paraplatin®+Paclitaxel, Paraplatin®+Vinblastine, Carboplatin (Paraplatin®)+Vincristine, Paraplatin®+Vindesine, Paraplatin®+Vinorelbine, Pemetrexed disodium+Gemcitabine, Platinol® (Cisplatin)+Vinblastine+Bleomycin (PVB), Prednisone+Methotrexate+Adriamycin+Cyclophosphamide+Etoposide (ProMACE), Procarbazine+Lomustine, Procarbazine+Lomustine+Vincristine, Procarbazine+Lomustine+Vincristine+Thioguanine, Procarbazine+Oncovin®+CCN-U+Cyclophosphamide (POCC), Quinine+Doxorubicin, Quinine+Mitoxantrone+Cytarabine, Thiotepa+Etoposide, Thiotepa+Busulfan+Cyclophosphamide, Thiotepa+Busulfan+Melphalan, Thiotepa+Etoposide+Carmustine, Thiotepa+Etoposide+Carboplatin, Topotecan+Paclitaxel, Trimetrexate+Leucovorin, Vinblastine+Doxorubicin+Thiotepa, Vinblastine+Bleomycin+Etoposide+Carboplatin, Vincristine+Lomustine+Prednisone, Vincristine (Oncovin®)+Adrinamycin®+Dexamethasone (VAD), Vincristine (Oncovin®)+Adriamycin®+Procarbazine (VAP), Vincristine+Dacfinomycin+Cyclophosphamide, and Vinorelbine+Gemcitabine.

In one embodiment, antibodies of the present invention may be administered in combination with a taxane and a platinum-based chemotherapeutic. In another embodiment, antibodies of the present invention may be administered in combination with a taxane and a platinum-based chemotherapeutic for the treatment of cancers that are resistant to individual chemotherapies. In a specific embodiment, antibodies of the present invention may be administered in combination with Docetaxel (Taxotere®) and Carboplatin (Paraplatin®, CBDCA). In another specific embodiment, antibodies of the present invention may be administered in combination with Docetaxel (Taxotere®) and Carboplatin (Paraplatin®, CBDCA) for the treatment of cancers that are resistant to individual chemotherapies.

In one embodiment, agonistic antibodies of the present invention may be administered in combination with a taxane and a platinum-based chemotherapeutic. In another embodiment, agonistic antibodies of the present invention may be administered in combination with a taxane and a platinum-based chemotherapeutic for the treatment of cancers that are resistant to individual chemotherapies. In a specific embodiment, agonistic antibodies of the present invention may be administered in combination with Docetaxel (Taxotere®) and Carboplatin (Paraplatin®, CBDCA). In another specific embodiment, agonistic antibodies of the present invention may be administered in combination with Docetaxel (Taxotere®) and Carboplatin (Paraplatin®, CBDCA) for the treatment of cancers that are resistant to individual chemotherapies.

In one embodiment, antibodies of the present invention may be administered in combination with a topoisomerase inhibitor and a fluoropyrimidine. In another embodiment, antibodies of the present invention may be administered in combination with a topoisomerase inhibitor and a fluoropyrimidine for the treatment of cancers that are resistant to individual chemotherapies. In a specific embodiment, antibodies of the present invention may be administered in combination with Irinotecan (Camptosar®, CPT-11, Topotecin®, CaptoCPT-1) and Fluorouracil (5-FU, Adrucil®). In another specific embodiment, antibodies of the present invention may be administered in combination with Irinotecan (Camptosar®, CPT-11, Topotecin®, CaptoCPT-1) and Fluorouracil (5-FU, Adrucil®) for the treatment of cancers that are resistant to individual chemotherapies.

In one embodiment, agonistic antibodies of the present invention may be administered in combination with a topoisomerase inhibitor and a fluoropyrimidine. In another embodiment, agonistic antibodies of the present invention may be administered in combination with a topoisomerase inhibitor and a fluoropyrimidine for the treatment of cancers that are resistant to individual chemotherapies. In a specific embodiment, agonistic antibodies of the present invention may be administered in combination with Irinotecan (Camptosar®, CPT-11, Topotecin®, CaptoCPT-1) and Fluorouracil (5-FU, Adrucil®). In another specific embodiment, agonistic antibodies of the present invention may be administered in combination with Irinotecan (Camptosar®, CPT-111, Topotecin®, CaptoCPT-1) and Fluorouracil (5-FU, Adrucil®) for the treatment of cancers that are resistant to individual chemotherapies.

In preferred embodiments, agonistic antibodies of the invention are administered in combination with one or more of the above-described combinations of therapeutic agents in the treatment, prevention, amelioration and/or cure of cancers.

In a specific embodiment, antibody and antibody compositions of the invention are administered in combination with CHOP (cyclophosphamide, doxorubicin, vincristine, and prednisone) or any combination of the components of CHOP. In another embodiment, antibody and antibody compositions of the invention are administered in combination with Rituximab. In a further embodiment, antibody and antibody compositions of the invention are administered with Rituximab and CHOP, or Rituximab and any combination of the components of CHOP.

In additional preferred embodiments, antibody compositions of the invention are administered in combination with Rituximab (Rituxan™) and/or Ibritumomab Tiuxetan (Zevalin™, e.g., either (In-111) Ibritumomab Tiuxetan or (Y-90) Ibritumomab Tiuxetan). In a specific embodiment, antibody compositions of the invention are administered in combination with Rituximab and/or Ibritumomab Tiuxetan for the treatment of non-Hodgkin's lymphoma.

In additional preferred embodiments, antibody compositions of the invention are administered in combination with imatinib mesylate (Gleevec®: 4-[(4-Methyl-1-piperazinyl)methyl]-N-[4-methyl-3-[[4-(3-pyridinyl)-2-pyrimidinyl]amino]-phenyl]benzamide methanesulfonate). In a specific embodiment, antibody compositions of the invention are administered in combination with imatinib mesylate for the treatment of chronic myelogenous leukemia.

In additional preferred embodiments, antibody compositions of the invention are administered in combination with bortezomib (Velcade™ [(1R)-3-methyl-1-[[(2S)-1-oxo-3-phenyl-2-[(pyrazinylcarbonyl) amino]propyl]amino]butyl] boronic acid). In a specific embodiment, antibody compositions of the invention are administered in combination with bortezomib for the treatment of multiple myeloma.

In additional preferred embodiments, antibody compositions of the invention are administered in combination with Alemtuzumab (Campath®). In a specific embodiment, antibody compositions of the invention are administered in combination with Alemtuzumab for the treatment of chronic lymphocytic leukemia.

In additional preferred embodiments, antibody compositions of the invention are administered in combination with fludarabine phosphate (Fludara®: 9H-Purin-6-amine, 2-fluoro-9-(5-O-phosphono-β-D-arabinofuranosyl) (2-fluoro-ara-AMP)). In a specific embodiment, antibody compositions of the invention are administered in combination with fludarabine phosphate for the treatment of chronic lymphocytic leukemia.

In additional preferred embodiments, the compositions of the invention are administered in combination with TRAIL polypeptides or fragments or variants thereof, particularly of the extracellular soluble domain of TRAIL.

In one embodiment, the compositions of the invention are administered in combination with other members of the TNF family or antibodies specific for TNF receptor family members. TNF, TNF-related or TNF-like molecules that may be administered with the compositions of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), TRAIL, AIM-II (International Publication No. WO 97/34911), APRIL (J. Exp. Med. 188(6):1185-1190), endokine-alpha (International Publication No. WO 98/07880), TR6 (International Publication No. WO 98/30694), OPG, and neutrokine-alpha (International Publication No. WO 98/18921, OX40, and nerve growth factor (NGF), and soluble forms of Fas, CD30, CD27, CD40 and 4-1BB, TR2 (International Publication No. WO 96/34095), DR3 (International Publication No. WO 97/35904), TR5 (International Publication No. WO 98/30693), TR6 (International Publication No. WO 98/30694), TR7 (International Publication No. WO 98/41629), TRANK, TR9 (International Publication No. WO 98/56892), TR10 (International Publication No. WO 98/54202), 312C2 (international Publication No. WO 98/06842), and TR12, and soluble forms CD154, CD70, and CD153.

In one embodiment, the antibody compositions of the invention are administered in combination with apoptosis inducing polypeptides. In a specific embodiment, antibodies of the invention are administered in combination with Smac (second mitochondria-derived activator of caspases) proteins, also known as DIABLO (direct LIP (inhibitor of apoptosis) binding protein with low pI) (GenBank Accession No. :NP_{—}063940 which is hereby incorporated by reference in its entirety). Smac is a 239 amino acid protein. The N-terminal 55 amino acids serve as a mitochondrial targeting sequence which is cleaved after import to the mitochondria. Apoptosis inducing polypeptides may be delivered using techniques known in the art. For example, one way to deliver Smac protein would be through the delivery of a nucleic acid encoding either the full length or mature form of Smac (amino acids 56-239 of GenBank Accession No. :NP_{—}063940, a cytosolic form that bypasses mitochondrial processing). Alternatively, antibody compositions of the invention may be administered in combination with cell permeable, synthetic Smac peptides which are capable of inhibiting IAP proteins (e.g., those containing amino acid residues 56-62 of GenBank Accession No. :NP-063940; AVPIAQK as described in Chai et al., (2000) Nature 406:855-862 and Fulda et al., (2002) Nature Medicine 8:808-815, both of which are hereby incorporated by reference in their entireties.

In one embodiment, an antibody composition of the invention is administered in combination with a histone deacetylase inhibitor. In another embodiment, an antibody compositions of the invention is administered in combination with inhibitors of ERK1/2.

(e.g., depsipeptide (e.g., FK-288 and FR901228), MS-275, and the triterpenoid 2-cyano-3,12-dioxooleana-1,9-dien-28-oic acid (CDDO) or other molecules related to CDDO, valproic acid, suberoylanilide hydroxamic acid (SAHA), pyroxamide, trapoxin, (depsipeptide), and N-acetyl dinaline (CI-994).

In one embodiment, an antibody composition of the invention is administered in combination with a Heat Shock Protein (HSP) Inhibitor. In a specific embodiment the HSP inhibitor is an HSP90 inhibitor, such as geldanamycin; geldanamycin analogs (e.g. 17-allylamino-17-demethoxy-geldanamycin (17AAG) and 17-aminogeldanamycin (17-AG)); and radicicol.

In one embodiment, an antibody composition of the invention is administered in combination with a synthetic retinoid (e.g. CD437, MX3350, ST1926).

In one embodiment, an antibody composition of the invention is administered in combination with a casein kinase II inhibitor, such as 5,6-dichloro-1-beta-D-ribofuranosylbenzimidazole (DRB); emodin; curcumin; quercetin; 4,5,6,7-tetrabromobenzotriazole (TBB) and derivatives thereof (e.g. 4,5,6,7-tetrabromo-2-(dimethylamino)benzimidazole (2c) and methylsulfanyl (8), isopropylamino (2e), and amino (2a) congeners); and LY-294002.

In one embodiment, an antibody composition of the invention is administered in combination with inhibitors of RAF kinase, such as, but not limited to, BAY 43-9006.

In one embodiment, an antibody composition of the invention is administered in combination with thapsigargin (THG).

In one embodiment, an antibody compositions of the invention is administered in combination with a survivin inhibitor, such as ICG-001.

In another embodiment, an antibody compositions of the invention is administered in combination with inhibitors of ERK1/2.

In another embodiment, an antibody compositions of the invention is administered in combination with proteasome inhibitors such as PS-341 (LDP-341, 26S proteasome inhibitor).

In another embodiment, an antibody compositions of the invention is administered in combination with a COX-2 inhibitor.

In specific embodiments antibodies of the present invention may be administered in combination with one or more therapeutic agents, as described above, in the treatment, prevention, amelioration and/or cure of hematological cancer (e.g., leukemia, acute lymphocytic leukemia, chronic lymphocytic leukemia, non-Hodgkin's lymphoma, multiple myeloma), colorectal cancer, lung cancer, brain cancer, skin cancer, breast cancer, prostate cancer, pancreatic cancer, hepatic cancer, ovarian cancer, as well as endothelioma, osteoblastoma, osteoclastoma, Ewing's sarcoma, and Kaposi's sarcoma.

In further particular embodiments, antibodies of the present invention are used to treat, ameliorate and/or prevent hematological cancers. Antibodies of the present invention may be used in combination with one or more surgical and/or radiological procedures and/or therapeutic agents to treat, ameliorate and/or prevent hematological cancers. Hematological cancers which may be treated using antibodies of the present invention include, but are not limited to, non-Hodgkin's lymphoma (e.g., small lymphocytic lymphoma, follicular center cell lymphoma, lymphoplasmacytoid lymphoma, marginal zone lymphoma, mantle cell lymphoma, immunoblastic lymphoma, burkitt's lymphoma, lymphoblastic lymphoma, peripheral T-cell lymphoma, anaplastic large cell lymphoma and intestinal T-cell lymphoma), leukemia, acute lymphocytic leukemia, chronic lymphocytic leukemia and plasma cell neoplasms including multiple myeloma.

In preferred embodiments, agonistic antibodies of the present invention are used to treat, ameliorate and/or prevent hematological cancers. Agonistic antibodies of the present invention may be used in combination with one or more surgical and/or radiological procedures and/or therapeutic agents to treat, ameliorate and/or prevent hematological cancers. Hematological cancers which may be treated using agonistic antibodies of the present invention include, but are not limited to, non-Hodgkin's lymphoma (e.g., small lymphocytic lymphoma, follicular center cell lymphoma, lymphoplasmacytoid lymphoma, marginal zone lymphoma, mantle cell lymphoma, immunoblastic lymphoma, burkitt's lymphoma, lymphoblastic lymphoma, peripheral T-cell lymphoma, anaplastic large cell lymphoma and intestinal T-cell lymphoma), leukemia, acute lymphocytic leukemia, chronic lymphocytic leukemia and plasma cell neoplasms including multiple myeloma.

In one preferred embodiment, agonistic antibodies of the invention are used to treat plasma cell neoplasms. In a specific embodiment, that plasma cell neoplasm is multiple myeloma.

In another preferred embodiment, agonistic antibodies of the invention are used to treat non-Hodgkin's lymphoma.

In another preferred embodiment, agonistic antibodies of the invention are used to treat leukemia. In a specific embodiment, that leukemia is acute lymphocytic leukemia. In another specific embodiment, that leukemia is chronic lymphocytic leukemia.

Antibodies of the present invention may be administered in combination with one or more surgical and/or radiological procedures useful in the treatment of hematological cancer including, but not limited to, bone marrow transplantation, external beam radiation and total body irradiation.

In preferred embodiments, agonistic antibodies of the invention are administered in combination with one or more surgical and/or radiological procedures useful in the treatment of hematological cancer including, but not limited to, bone marrow transplantation, external beam radiation and total body irradiation.

In one preferred embodiment, agonistic antibodies of the present invention may be administered in combination with one or more surgical and/or radiological procedures useful in the treatment of multiple myeloma including, but not limited to, allogeneic bone marrow transplantation and peripheral stem cell support.

In another preferred embodiment, agonistic antibodies of the present invention may be administered in combination with one or more surgical and/or radiological procedures useful in the treatment of non-Hodgkin's lymphoma including, but not limited to, allogeneic bone marrow transplantation and peripheral stem cell support.

In further specific embodiments, agonistic antibodies of the present invention may be administered in combination with one or more surgical and/or radiological procedures useful in the treatment of leukemia including, but not limited to, allogeneic bone marrow transplantation and peripheral stem cell support. In one specific preferred embodiment, agonistic antibodies of the invention are used to treat acute lymphocytic leukemia (ALL). In another specific preferred embodiment, agonistic antibodies of the invention are used to treat chronic lymphocytic leukemia (CLL).

Antibodies of the present invention may be administered in combination with one or more therapeutic agents useful in the treatment of multiple myeloma including, but not limited to, Alkylating agents, Anthracyclines, Carmustine (DTI-015, BCNU, BiCNU, Gliadel Wafer®), Cyclophosphamide (Cytoxan®, Neosar®, CTX), Dexamethasone (Decadron®), Doxorubicin (Adriamycin®, Doxil®, Rubex®), Melphalan (L-PAM, Alkeran®, Phenylalanine mustard), Prednisone, Thalidomide and thalidomide analogs, including but not limited to, lenalidomide (CC-5013, REVLIMID) and CC4047 (ACTIMID), and Vincristine (Oncovorin®, Onco TCS®, VCR, Leurocristine®).

In preferred embodiments, agonistic antibodies of the invention are administered in combination with one or more of the above-described therapeutic agents in the treatment, amelioration and/or prevention of multiple myeloma.

Preferred combinations of therapeutic agents useful in the treatment of multiple myeloma which may be administered in combination with antibodies of the present invention include, but are not limited to, Cyclophosphamide+Prednisone, Melphalan+Prednisone (MP), Vincristine+Adriamycin®+Dexamethasone (VAD), Vincristine+Carmustine+Melphalan+Cyclophosphamide+Prednisone (VBMCP; the M2 protocol), and Vincrisbine+Melphalan+Cyclophosphamide+Prednisone alternating with Vincristine+Carmustine+Doxorubicin+Prednisone (VMCP/VBAP).

In preferred embodiments, agonistic antibodies of the invention are administered in combination with one or more of the above-described therapeutic agent combinations in the treatment, amelioration and/or prevention of multiple myeloma.

Antibodies of the present invention may be administered in combination with one or more therapeutic agents useful in the treatment of non-Hodgkin's lymphoma including, but not limited to, 2-chlorodeoxyadenosine, Amifostine (Ethyol®, Ethiofos®, WR-272), Bexarotene (Targretin®, Targretin gel®, Targretin oral®, LGD1069), Bleomycin (Blenoxane®), Busulfan (Busulfex®, Myleran®), Carboplatin (Paraplatin®, CBDCA), Carmustine (DTI-015, BCNU, BiCNU, Gliadel Wafer®), Chlorambucil (Leukeran®), Cisplatin (Platinol®, CDDP), Cladribine (2-CdA, Leustatin®), Cyclophosphamide (Cytoxan®, Neosar®, CTX), Cytarabine (Cytosar-U®, ara-C, cytosine arabinoside, DepoCyt®), Dacarbazine (DTIC), Daunorubicin (Daunomycin, DaunoXome®, Daunorubicin®, Cerubidine®), Denileukin diftitox (Ontak®), Dexamethasone (Decadron®), Dolasetron mesylate (Anzemet®), Doxorubicin (Adriamycin®, Doxil®, Rubex®), Erythropoietin (EPO®, Epogen®, Procrit®), Etoposide phosphate (Etopophose), Etoposide (VP-16, Vepesid®), Fludarabine (Fludara®, FAMP), Granisetron (Kytril®), Hydrocortisone, Idarubicin (Idamycin®, DMDR, IDA), Ifosfamide (IFEX®), Interferon alpha (Mlfaferone®, Alpha-IF®), Interferon alpha 2a (Intron A®), Mechlorethamine (Nitrogen Mustard, HN₂, Mustargen®)), Melphalan (L-PAM, Alkeran®, Phenylalanine mustard), Methotrexate® (MTX, Mexate®, Folex®), Methylprednisolone (Solumedrol®), Mitoxantrone (Novantrone®, DHAD), Ondansetron (Zofran®), Pentostatin (Nipent®, 2-deoxycoformycin), Perfosfamide (4-hydroperoxycyclophosphamide, 4-HC), Prednisone, Procarbazine (Matulane®), Rituximab® (Rituxan®, anti-CD20 MAb), Thiotepa (triethylenethiophosphaoramide, Thioplex®), Topotecan (Hycamtin®, SK&F-104864, NSC-609699, Evotopin®), Vinblastine (Velban®, VLB), Vincristine (Oncovin®, Onco TCS®, VCR, Leurocristine®) and Vindesine (Eldisine®, Fildesin®).

In preferred embodiments, agonistic antibodies of the invention are administered in combination with one or more of the above-described therapeutic agents in the treatment, amelioration and/or prevention of non-Hodgkin's lymphoma.

Preferred combinations of therapeutic agents useful in the treatment of non-Hodgkin's lymphoma which may be administered in combination with antibodies of the present invention include, but are not limited to, Adriamycin®+Blenoxane+Vinblastine+Dacarbazine (ABVD), Anti-idiotype therapy (BsAb)+Interferon alpha, Anti-idiotype therapy (BsAb)+Chlorambucil, Anti-idiotype therapy (BsAb)+Interleukin-2, BCNU (Carmustine)+Etoposide+Ara-C (Cytarabine)+Melphalen (BEAM), Bleomycin+Etoposide+Adriamycin+Cyclophosphamide+Vincristine+Procarbazine+Prednisone (BEACOPP), Bryostatin+Vincristine, Cyclophosphamide+BCNU (Carmustine)+VP-16 (Etoposide) (CBV), Cyclophosphamide+Vincristine+Prednisone (CVP), Cyclophosphamide+Adriamycin® (Hydroxyldaunomycin)+Vincristine (Oncovorin)+Prednisone (CHOP), Cyclophosphamide+Novantrone® (Mitoxantrone)+Vincristine (Oncovorin)+Prednisone (CNOP), Cyclophosphamide+Doxorubicin+Teniposide+Prednisone, Cyclophosphamide+Adriamycin® (Hydroxyldaunomycin)+Vincristine (Oncovorin)+Prednisone+Rituximab (CHOP+Rituximab), Cyclophosphamide+Doxorubicin+Teniposide+Prednisone+Interferon alpha, Cytarabine+Bleomycin+Vincristine+Methotrexate (CytaBOM), Dexamethasone+Cytarabine+Cisplatin (DHAP), Dexamethasone+Ifosfamide+Cisplatin+Etoposide (DICE), Doxorubicin+Vinblastine+Mechlorethamine+Vincristine+Bleomycin+Etoposide+Prednisone (Stanford V), Etoposide+Vinblastine+Adriamycin (EVA), Etoposide+Methylprednisone+Cytarabine+Cisplatin (ESHAP), Etoposide+Prednisone+Ifosfamide+Cisplatin (EPIC), Fludarabine, Mitoxantrone+Dexamethasone (FMD), Fludarabine, Dexamethasone, Cytarabine (ara-C), +Cisplatin (Platinol®) (FluDAP), Ifosfamide+Cisplatin+Etoposide (ICE), Mechlorethamine+Oncovin® (Vincristine)+Procarbazine+Prednisone (MOPP), Mesna+Ifosfamide+Idarubicin+Etoposide (MIZE), Methotrexate with leucovorin rescue+Bleomycin+Adriamycin+Cyclophosphamide+Oncovorin+Dexamethasone (m-BACOD), Prednisone+Methotrexate+Adriamycin+Cyclophosphamide+Etoposide (ProMACE), Thiotepa+Busulfan+Cyclophosphamide, Thiotepa+Busulfan+Melphalan, Topotecan+Paclitaxel, and Vincristine (Oncovin®)+Adriamycin®+Dexamethasone (VAD).

In preferred embodiments, agonistic antibodies of the invention are administered in combination with one or more of the above-described therapeutic agent combinations in the treatment, amelioration and/or prevention of non-Hodgkin's lymphoma.

Further examples of therapeutic agents useful in the treatment of non-Hodgkin's lymphoma which may be administered in combination with antibodies of the present invention include, but are not limited to, A007 (44′-dihydroxybenzophenone-2,4-dinitrophenylhydrazone), AG-2034 (AG-2024, AG-2032, GARFT [glycinamide ribonucleoside transformylase] inhibitor), Aldesleukin (IL-2, Proleukin®), Alemtuzumab (Campath®), Alitretinoin (Panretin®, LGN-1057), Altretamine (Hexalen®, hexamethylmelamine, Hexastat®), Aminocamptothecin (9-AC, 9-Aminocamptothecin, NSC 603071), Anti-CD19/CD3 MAb (anti-CD19/CD3 scFv, anti-NHL MAb), Anti-idiotype therapy (BsAb), Arabinosylguanine (Ara-G, GW506U78), Arsenic trioxide (Trisenox®, ATO), B43-Genistein (anti-CD19 Ab/genistein conjugate), B7 antibody conjugates, Betathine (Beta-LT), BLyS antagonists, Bryostatin-1 (Bryostatin®, BMY-45618, NSC-339555), CHML (Cytotropic Heterogeneous Molecular Lipids), Clofarabine (chloro-fluoro-araA), Daclizumab (Zenapax®), Depsipeptide (FR901228, FK228), Dolastatin-10 (DOLA-10, NSC-376128), Epirubicin (Ellence®, EPI, 4′ epi-doxorubicin), Epratuzumab (Lymphocide®, humanized anti-CD22, HAT), Fly3/flk2 ligand (Mobista®), G3139 (Genasense®, GentaAnticode®, Bcl-2 antisense), HulD10 (anti-HLA-DR MAb, SMART 1D10), HumaLYM (anti-CD20 MAb), Ibritumomab tiuxetan (Zevalin®), Interferon gamma (Gamma-interferon, Gamma 1000, Gamma-1F), Irinotecan (Camptosar®, CPT-11, Topotecin®, CaptoCPT-1), ISIS-2053, ISIS-3521 (PKC-alpha antisense), Lmb-2 immunotoxin (anti-CD25 recombinant immuno toxin, anti-Tac(Fv)-PE38), Leuvectin® (cytofectin+IL-2 gene, IL-2 gene therapy), Lym-1 (131-I LYM-1), Lymphoma vaccine (Genitope), Nelarabine (Compound 506, U78), Neugene compounds (Oncomyc-NG®, Resten-NG®, myc antisense), NovoMAb-G2 scFv (NovoMAb-G2 IgM), O6-benzylguanine (BG, Procept®), Oxaliplatin (Eloxatine®, Eloxatin®), Paclitaxel (Paxene®, Taxol®), Paclitaxel-DHA (Taxoprexin®), Peldesine (BCX-34, PNP inhibitor), Rebeccamycin and Rebeccamycin analogues, SCH-66336, Sobuzoxane (MST-16, Perazolin®), SU5416 (Semaxanib®, VEGF inhibitor), TER-286, Thalidomide and thalidomide analogs, including but not limited to, lenalidomide (CC-5013, REVLIMIND) and CC-4047 (ACTIMID), TNP-470 (AGM-1470), Tositumomab (Bexxar®), Valspodar (PSC 833), Vaxid (B-cell lymphoma DNA vaccine), Vinorelbine (Navelbine®), WF10 (macrophage regulator) and XR-9576 (XR-9351, P-glycoprotein/MDR inhibitor).

In preferred embodiments, agonistic antibodies of the invention are administered in combination with one or more of the above-described therapeutic agents in the treatment, amelioration and/or prevention of non-Hodgkin's lymphoma.

Antibodies of the present invention may be administered in combination with one or more therapeutic agents useful in the treatment of acute lymphocytic leukemia including, but not limited to, Amsacrine, Carboplatin (Paraplatin®, CBDCA), Carmustine (DTI-015, BCNU, BiCNU, Gliadel Wafer®), Cholecaliferol, Cyclophosphamide (Cytoxan®, Neosar®, CTX), Cytarabine (Cytosar-U®, ara-C, cytosine arabinoside, DepoCyt®), Daunorubicin (Daunomycin, DaunoXome®, Daunorubicin®, Cerubidine®), Dexamethasone (Decadron®), Doxorubicin (Adriamycin®, Doxil®, Rubex®), Etoposide (VP-16, Vepesid®), Filgrastam® (Neupogen®, G-CSF, Leukine®), Fludarabine (Fludara®, FAMP), Idarubicin (Idamycin®, DMDR, IDA), Ifosfamide (IFEX®), Imatinib mesylate (STI-571, Imatinib®, Glivec®, Gleevec®, Abl tyrosine kinase inhibitor), Interferon gamma (Gamma-interferon, Gamma 1001, Gamma-1F), L-asparaginase (Elspar®, Crastinin®, Asparaginase medac®, Kidrolase®), Mercaptopurine (6-mercaptopurine, 6-MP), Methotrexate® (MTX, Mexate®, Folex®), Mitoxantrone (Novantrone®, DHAD), Pegaspargase® (Oncospar®), Prednisone, Retinoic acid, Teniposide (VM-26, Vumon®), Thioguanine (6-thioguanine, 6-TG), Topotecan (Hycamtin®, SK&F-104864, NSC-609699, Evotopin®), Tretinoin (Retin-A®, Atragen®, ATRA, Vesanoid®) and Vincristine (Oncovorin®, Onco TCS®, VCR, Leurocristine®).

In preferred embodiments, agonistic antibodies of the invention are administered in combination with one or more of the above-described therapeutic agents in the treatment, amelioration and/or prevention of acute lymphocytic leukemia.

Further examples of therapeutic agents useful in the treatment of acute lymphocytic leukemia which may be administered in combination with antibodies of the present invention include, but are not limited to, Aminocamptothecin (9-AC, 9-Aminocamptothecin, NSC 603071), Aminopterin, Annamycin (AR-522, annamycin LF, Aronex®), Arabinosylguanine (Ara-G, GW506U78, Nelzarabine®), Arsenic trioxide (Trisenox®, ATO, Atrivex®), B43-Genistein (anti-CD19 Ab/genistein conjugate), B43-PAP (anti-CD19 Ab/pokeweed antiviral protein conjugate), Cordycepin, CS-682, Decitabine (5-aza-2′-deoxyytidine), Dolastatin-10 (DOLA-10, NSC-376128), G3139 (Genasense®, GentaAnticode®, Bcl-2 antisense), Irofulven (MGI-114, Ivofulvan, Acylfulvene analogue), MS-209, Phenylbutyrate, Quinine, TNP470 (AGM-1470, Fumagillin), Trimetrexate (Neutrexin®), Troxacitabine (BCH-204, BCH-4556, Troxatyl®), UCN-01 (7-hydroxystaurosporine), WHI-P131 and WT1 Vaccine.

In preferred embodiments, agonistic antibodies of the invention are administered in combination with one or more of the above-described therapeutic agents in the treatment, amelioration and/or prevention of acute lymphocytic leukemia.

Preferred combinations of therapeutic agents useful in the treatment of acute lymphocytic leukemia which may be administered in combination with antibodies of the present invention include, but are not limited to, Carboplatin+Mitoxantrone, Carmustine+Cyclophosphamide+Etoposide, Cytarabine+Daunorubicin, Cytarabine+Doxorubicin, Cytarabine+Idarubicin, Cytarabine+Interferon gamma, Cytarabine+L-asparaginase, Cytarabine+Mitoxantrone, Cytarabine+Fludarabine and Mitoxantrone, Etoposide+Cytarabine, Etoposide+Ifosfamide, Etoposide+Mitoxantrone, Ifosfamide+Etoposide+Mitoxantrone, Ifosfamide+Teniposide, Methotrexate+Mercaptopurine, Methotrexate+Mercaptopurine+Vincristine+Prednisone, Phenylbutyrate+Cytarabine, Phenylbutyrate+Etoposide, Phenylbutyrate+Topotecan, Phenylbutyrate+Tretinoin, Quinine+Doxorubicin, Quinine+Mitoxantrone+Cytarabine, Thioguanine+Cytarabine+Amsacrine, Thioguanine+Etoposide+Idarubicin, Thioguanine+Retinoic acid+Cholecaliferol, Vincristine+Prednisone, Vincristine+Prednisone and L-asparaginase, Vincristine+Dexamethasone/Prednisone+Asparaginase+Daunorubicin/Doxorubicin, Vincristine+Dexamethasone/Prednisone+Asparaginase+Daunorubicin/Doxorubicin+Filgrastim, Vincristine+Dexamethasone/Prednisone+Asparaginase+Daunorubicin/Doxorubicin+Cyclophosphamide+Methotrexate, and Vincristine+Dexamethasone/Prednisone+Asparaginase+Daunorubicin/Doxorubicin+Cyclophosphamide+Methotrexate+Filgrastim.

In preferred embodiments, agonistic antibodies of the invention are administered in combination with one or more of the above-described therapeutic agent combinations in the treatment, amelioration and/or prevention of acute lymphocytic leukemia.

Antibodies of the present invention may be administered in combination with one or more therapeutic agents useful in the treatment of chronic lymphocytic leukemia including, but not limited to, Chlorambucil (Leukeran®), Cladribine (2-CdA, Leustatin®), Cyclophosphamide (Cytoxan®, Neosar®, CTX), Cytarabine (Cytosar-U®, ara-C, cytosine arabinoside, DepoCyt®, cytarabine ocfosfate, ara-CMP), Doxorubicin (Adriamycin®, Doxil®, Rubex®), Fludarabine (Fludara®, FAMP), Pentostatin (Nipent®, 2-deoxycoformycin), Prednisone and Vincristine (Oncovorin®, Onco TCS®, VCR, Leurocristine®).

In preferred embodiments, agonistic antibodies of the invention are administered in combination with one or more of the above-described therapeutic agents in the treatment, amelioration and/or prevention of chronic lymphocytic leukemia.

Further examples of therapeutic agents useful in the treatment of chronic lymphocytic leukemia which may be administered in combination with antibodies of the present invention include, but are not limited to, Alemtuzumab (Campath®), Aminocamptothecin (9-AC, 9-Aminocamptothecin, NSC 603071), Aminopterin, Annamycin (AR-522, annamycin LF, Aronex®), Arabinosylguanine (Ara-G, GW506U78, Nelzarabine®, Compound 506U78), Arsenic trioxide (Trisenox®, ATO, Atrivex®), Bryostatin-1 (Bryostatin®, BMY-45618, NSC-339555), CS-682, Dolastatin-10 (DOLA-10, NSC-376128), Filgrastim (Neupogen®, G-CSF, Leukine), Flavopiridol (NSC-649890, HMR-1275), G3139 (Genasense®, GentaAnticode®, Bcl-2 antisense), Irofulven (MGI-114, Ivofulvan, Acylfulvene analogue), MS-209, Phenylbutyrate, Rituximab® (Rituxan®, anti-CD20 MAb), Thalidomide and thalidomide analogs, including but not limited to, lenalidomide (CC-5013, REVLIMID) and CC4047 (ACTIMID), Theophylline, TNP470 (AGM-1470, Fumagillin), UCN-01 (7-hydroxystaurosporine) and WHI-P131.

In preferred embodiments, agonistic antibodies of the invention are administered in combination with one or more of the above-described therapeutic agents in the treatment, amelioration and/or prevention of chronic lymphocytic leukemia.

Preferred combinations of therapeutic agents useful in the treatment of chronic lymphocytic leukemia which may be administered in combination with antibodies of the present invention include, but are not limited to, Fludarabine+Prednisone, and Cyclophosphamide+Doxorubicin+Vincristine+Prednisone (CHOP).

In preferred embodiments, agonistic antibodies of the invention are administered in combination with one or more of the above-described therapeutic agent combinations in the treatment, amelioration and/or prevention of chronic lymphocytic leukemia.

In further particular embodiments, antibodies of the present invention are used to treat, ameliorate and/or prevent colorectal cancer. Antibodies of the present invention may be used in combination with one or more surgical and/or radiological procedures and/or therapeutic agents to treat, ameliorate and/or prevent colorectal cancer. Colorectal cancers which may be treated using antibodies of the present invention include, but are not limited to, colon cancer (e.g., early stage colon cancer (stage I and II), lymph node positive colon cancer (stage III), metastatic colon cancer (stage IV)) and rectal cancer.

In preferred embodiments, agonistic antibodies of the present invention are used to treat, ameliorate and/or prevent colorectal cancer. Agonistic antibodies of the present invention may be used in combination with one or more surgical and/or radiological procedures and/or therapeutic agents to treat, ameliorate and/or prevent colorectal cancer. Colorectal cancers which may be treated using agonistic antibodies of the present invention include, but are not limited to, colon cancer (e.g., early stage colon cancer (stage I and II), lymph node positive colon cancer (stage III), metastatic colon cancer (stage I)) and rectal cancer.

In one preferred embodiment, agonistic antibodies of the invention are used to treat colon cancer.

Antibodies of the present invention may be administered in combination with one or more therapeutic agents useful in the treatment of colorectal cancer including, but not limited to, Capecitabine (Xeloda®, Doxifluridine®, oral 5-FU), Fluorouracil (5-FU, Adrucil®, Fluoroplex®, Efudex®), Irinotecan (Camptosar®, CPT-11, Topotecin®, CaptoCPT-1), Leucovorin (Leucovorin®, Wellcovorin®), and Levamisole (Ergamisol®).

In one embodiment, antibodies of the present invention may be administered in combination with a topoisomerase inhibitor. In another embodiment, antibodies of the present invention may be administered in combination with a topoisomerase inhibitor for the treatment of colon cancer that is resistant to individual chemotherapies. In a specific embodiment, antibodies of the present invention may be administered in combination with Irinotecan (Camptosar®, CPT-11, Topotecin®, CaptoCPT-1). In a specific embodiment, antibodies of the present invention may be administered in combination with Irinotecan (Camptosar®, CPT-11, Topotecin®, CaptoCPT-1) for the treatment of colon cancer that is resistant to individual chemotherapies.

In one embodiment, agonistic antibodies of the present invention may be administered in combination with a topoisomerase inhibitor. In another embodiment, agonistic antibodies of the present invention may be administered in combination with a topoisomerase inhibitor for the treatment of colon cancer that is resistant to individual chemotherapies. In a specific embodiment, agonistic antibodies of the present invention may be administered in combination with Irinotecan (Camptosar®, CPT-11, Topotecin®, CaptoCPT-1). In a specific embodiment, agonistic antibodies of the present invention may be administered in combination with Irinotecan (Camptosar®, CPT-11, Topotecin®, CaptoCPT-1) for the treatment of colon cancer that is resistant to individual chemotherapies.

In one embodiment, antibodies of the present invention may be administered in combination with a fluoropyrimidine. In another embodiment, antibodies of the present invention may be administered in combination with a fluoropyrimidine for the treatment of colon cancer that is resistant to individual chemotherapies. In another specific embodiment, antibodies of the invention may be administered in combination with Fluorouracil (5-FU, Adrucil®). In another specific embodiment, antibodies of the present invention may be administered in combination with Fluorouracil (5-FU, Adrucil®1) for the treatment of colon cancer that is resistant to individual chemotherapies.

In one embodiment, agonistic antibodies of the present invention may be administered in combination with a fluoropyrimidine. In another embodiment, agonistic antibodies of the present invention may be administered in combination with a fluoropyrimidine for the treatment of colon cancer that is resistant to individual chemotherapies. In another specific embodiment, agonistic antibodies of the invention may be administered in combination with Fluorouracil (5-FU, Adrucil®). In another specific embodiment, agonistic antibodies of the present invention may be administered in combination with Fluorouracil (5-FU, Adrucil®) for the treatment of colon cancer that is resistant to individual chemotherapies.

In preferred embodiments, agonistic antibodies of the invention are administered in combination with one or more of the above-described therapeutic agents in the treatment, amelioration and/or prevention of colorectal cancers.

Preferred combinations of therapeutic agents useful in the treatment of colorectal cancer which may be administered in combination with antibodies of the present invention include, but are not limited to, Fluorouracil+Leucovorin, and Fluorouracil+Levamisole.

In one embodiment, antibodies of the present invention may be administered in combination with a topoisomerase inhibitor and a fluoropyrimidine. In another embodiment, antibodies of the present invention may be administered in combination with a topoisomerase inhibitor and a fluoropyrimidine for the treatment of colon cancer, that are resistant to individual chemotherapies. In a specific embodiment, antibodies of the present invention may be administered in combination with Irinotecan (Camptosar®, CPT-11, Topotecin®, CaptoCPT-1) and Fluorouracil (5-FU, Adrucil®). In another specific embodiment, antibodies of the present invention may be administered in combination with Irinotecan (Camptosar®, CPT-11, Topotecin®, CaptoCPT-1) and Fluorouracil (5-FU, Adrucil®) for the treatment of colon cancer that is resistant to individual chemotherapies.

In one embodiment, agonistic antibodies of the present invention may be administered in combination with a topoisomerase inhibitor and a fluoropyrimidine. In another embodiment, agonistic antibodies of the present invention may be administered in combination with a topoisomerase inhibitor and a fluoropyrimidine for the treatment of colon cancer, that are resistant to individual chemotherapies. In a specific embodiment, agonistic antibodies of the present invention may be administered in combination with Irinotecan (Camptosar®, CPT-11, Topotecin®, CaptoCPT-1) and Fluorouracil (5-FU, Adrucil®). In another specific embodiment, agonistic antibodies of the present invention may be administered in combination with Irinotecan (Camptosar®, CPT-11, Topotecin®, CaptoCPT-1) and Fluorouracil (5-FU, Adrucil®) for the treatment of colon cancer, that are resistant to individual chemotherapies.

In preferred embodiments, agonistic antibodies of the invention are administered in combination with one or more of the above-described therapeutic agent combinations in the treatment, amelioration and/or prevention of colorectal cancers.

Further examples of therapeutic agents useful in the treatment of colorectal cancer which may be administered in combination with antibodies of the present invention include, but are not limited to, Aminocamptothecin (9-AC, 9-Aminocamptothecin, NSC 603071), Aplidine (Aplidin®, Aplidina°), Bevacizumab® (Anti-VEGF monoclonal antibody, rhuMAb-VEGF), C225 (IMC-225, EGFR inhibitor, Anti-EGFr MAb, Cetuximab®), C242-DMI (huC242-DMI), CC49-zeta gene therapy, CEA-cide® (Labetuzumab®, Anti-CEA monoclonal antibody, hMN-14), CeaVac® (MAb 3H1), CP-609754, CTP-37 (Avicine®, hCG blocking vaccine), Declopramide (Oxi-104), Eniluracil (776c85), F19 (Anti-FAP monoclonal antibody, iodinated anti-FAP MAb), FMdC (KW-2331, MDL-101731), FUDR (Floxuridine®), Gemcitabine (Gemto®, Gemzar®), Herceptin® (Trastuzumab®, Anti-HER-2 monoclonal antibody, Anti-EGFR-2 MAb), Intoplicine (RP 60475), L-778123 (Ras inhibitors), Leuvectin® (cytofectin+IL-2 gene, IL-2 gene therapy), MN-14 (Anti-CEA immunoradiotherapy, ¹³¹I-MN-14, ¹⁸⁸Re-MN-14), OncoVAX-CL, OncoVAX-CL-Jenner (GA-733-2 vaccine) Orzel® (Tegafur+Uracil+Leucovorin), Oxaliplatin (Eloxatine®, Eloxatin®), Paclitaxel-DHA (Taxoprexin®), Pemetrexed disodium (Alimta®, MTA, multitargeted antifolate, LY 231514), R115777 (Zamestra®), Raltitrexed (Tomudex®, ZD-1694), SCH 66336, SU5416 (Semaxanib®, VEGF inhibitor), Tocladesine (8-Cl-cAMP), Trimetrexate (Neutrexin®), TS-1, and ZD-9331.

In preferred embodiments, agonistic antibodies of the invention are administered in combination with one or more of the above-described therapeutic agents in the treatment, amelioration and/or prevention of colorectal cancers.

Further exemplary combinations of therapeutic agents useful in the treatment of colorectal cancer which may be administered in combination with antibodies of the present invention include, but are not limited to, Aminocamptothecin+G-CSF, Bevacizumab®+Fluorouracil, Bevacizumab®+Leucovorin, Bevacizumab®+Fluorouracil+Leucovorin, Cyclophosphamide+SCH 6636, Fluorouracil+CeaVac®, Fluorouracil+Oxaliplatin, Fluorouracil+Raltitrexed, Fluorouracil+SCH 6636, Fluorouracil+Trimetrexate, Fluorouracil+Leucovorin+Oxaliplatin, Fluorouracil+Leucovorin+Trimetrexate, Irinotecan+C225 (Cetuximab®), Oncovin®+SCH 6636, Oxaliplatin+Leucovorin, Paclitaxel+SCH 6636, Pemetrexed disodium+Gemcitabine, and Trimetrexate+Leucovorin.

In preferred embodiments, agonistic antibodies of the invention are administered in combination with one or more of the above-described therapeutic agent combinations in the treatment, amelioration and/or prevention of colorectal cancers.

In further particular embodiments, antibodies of the present invention are used to treat, ameliorate and/or prevent lung cancer. Antibodies of the present invention may be used in combination with one or more surgical and/or radiological procedures and/or therapeutic agents to treat, ameliorate and/or prevent lung cancer. Lung cancer which may be treated using antibodies of the present invention includes, but is not limited to, non-small cell lung cancer (NSCLC) including early stage NSCLC (i.e., Stage IA/IB and Stage IIA/IIB), Stage IIIA NSCLC, Stage IIA(unresectable)/IIIB NSCLC and Stage IV NSCLC, small cell lung cancer (SCLC) including limited stage SCLC and extensive stage SCLC as well as Malignant Pleural Mesothelioma.

In preferred embodiments, agonistic antibodies of the present invention are used to treat, ameliorate and/or prevent lung cancer. Agonistic antibodies of the present invention may be used in combination with one or more surgical and/or radiological procedures and/or therapeutic agents to treat, ameliorate and/or prevent lung cancer. Lung cancer which may be treated using agonistic antibodies of the present invention includes, but is not limited to, non-small cell lung cancer (NSCLC) including early stage NSCLC (i.e., Stage IA/IB and Stage IIA/IIB), Stage IIIA NSCLC, Stage IIA(unresectable)/IIIB NSCLC and Stage IV NSCLC, small cell lung cancer (SCLC) including limited stage SCLC and extensive stage SCLC as well as Malignant Pleural Mesothelioma.

In one preferred embodiment, agonistic antibodies of the invention are used to treat non-small cell lung cancers.

Antibodies of the present invention may be administered in combination with one or more therapeutic agents useful in the treatment of lung cancer including, but not limited to, BAY 43-9006 (Raf kinase inhibitor), Carboplatin (Paraplatin®, CBDCA), Chlorambucil (Leukeran®), Cisplatin (Platinol®, CDDP), Cisplatin-epinephrine gel (IntraDose®, FocaCist®), Cyclophosphamide (Cytoxan®, Neosar®, CTX), Docetaxel (Taxotere®, Taxane®), Doxorubicin (Adriamycin®, Doxil®, Rubex®), Edatrexate, Epirubicin (Ellence®, EPI, 4′ epi-doxorubicin), Etoposide phosphate (Etopophos®), Etoposide (VP-16, Vepesid®), Gemcitabine (Gemto®, Gemzar®), Herceptin® (Trastuzumab®, Anti-HER-2 monoclonal antibody, Anti-EGFR-2 MAb), Ifosfamide (IFEXS), Irinotecan (Camptosar®, CPT-11, Topotecin®), CaptoCPT-1), Lomustine (CCNU®, CeeNU®), Mechlorethamine (Nitrogen Mustard, HN₂, Mustargen®), Melphalan (L-PAM, Alkeran®, Phenylalanine mustard), Methotrexate® (MTX, Mexate®, Folex®), Mitomycin C (Mitomycin®, Mutamycin®, Mito Extra®), Paclitaxel (Paxene®, Taxol®), Paclitaxel-DHA (Taxoprexin®), Porfimer sodium (Photofrin®), Procarbazine (Matulane®), SKI-2053R (NSC-D644591), Teniposide (VM-26, Vumon®), Topotecan (Hycamtin®, SK&F-104864, NSC-609699, Evotopin®), Vinblastine (Velban®, VLB), Vincristine (Oncovin®, Onco TCS®, VCR, Leurocristine®), Vindesine (Eldisine®, Fildesin®), and Vinorelbine (Navelbine®).

In preferred embodiments, agonistic antibodies of the invention are administered in combination with one or more of the above-described therapeutic agents in the treatment, amelioration and/or prevention of lung cancers.

In one embodiment, antibodies of the present invention may be administered in combination with a taxane. In another embodiment, antibodies of the present invention may be administered in combination with a taxane for the treatment of lung cancers, such as non-small cell lung cancer, that are resistant to individual chemotherapies. In a specific embodiment, antibodies of the present invention may be administered in combination with Docetaxel (Taxotere®). In a specific embodiment, antibodies of the present invention may be administered in combination with Docetaxel (Taxotere®) for the treatment of lung cancers, such as non-small cell lung cancer, that are resistant to individual chemotherapies.

In one embodiment, agonistic antibodies of the present invention may be administered in combination with a taxane. In another embodiment, agonistic antibodies of the present invention may be administered in combination with a taxane for the treatment of lung cancers, such as non-small cell lung cancer, that are resistant to individual chemotherapies. In a specific embodiment, agonistic antibodies of the present invention may be administered in combination with Docetaxel (Taxotere®). In a specific embodiment, agonistic antibodies of the present invention may be administered in combination with Docetaxel (Taxotere®) for the treatment of lung cancers, such as non-small cell lung cancer, that are resistant to individual chemotherapies.

In one embodiment, antibodies of the present invention may be administered in combination with a platinum-based chemotherapeutic. In another embodiment, antibodies of the present invention may be administered in combination with a platinum-based chemotherapeutic for the treatment of lung cancers, such as non-small cell lung cancer, that are resistant to individual chemotherapies. In another specific embodiment, antibodies of the invention may be administered in combination with Carboplatin (Paraplatin®, CBDCA). In another specific embodiment, antibodies of the present invention may be administered in combination with Carboplatin (Paraplatin®, CBDCA) for the treatment of lung cancers, such as non-small cell lung cancer, that are resistant to individual chemotherapies.

In one embodiment, agonistic antibodies of the present invention may be administered in combination with a platinum-based chemotherapeutic. In another embodiment, agonistic antibodies of the present invention may be administered in combination with a platinum-based chemotherapeutic for the treatment of lung cancers, such as non-small cell lung cancer, that are resistant to individual chemotherapies. In another specific embodiment, agonistic antibodies of the invention may be administered in combination with Carboplatin (Paraplatin®, CBDCA). In another specific embodiment, agonistic antibodies of the present invention may be administered in combination with Carboplatin (Paraplatin®, CBDCA) for the treatment of lung cancers, such as non-small cell lung cancer, that are resistant to individual chemotherapies.

Further examples of therapeutic agents useful in the treatment of lung cancer which may be administered in combination with antibodies of the present invention include, but are not limited to, ABX-EGF (anti-EGFr MAb), Acetyldinaline (CI-994), AG-2034 (AG-2024, AG-2032, GARFT [glycinamide ribonucleoside transformylase] inhibitor), Alanosine, Aminocamptothecin (9-AC, 9-Aminocamptothecin, NSC 603071), Angiostatin, Aplidine (Aplidin®, Aplidina®), BBR 3464, Bexarotene (Targretin®, LGD1069), BIBH-1 (Anti-FAP MAb), BIBX-1382, BLP-25 (MUC-1 peptide), Bryostatin-1 (Bryostatin®, BMY45618, NSC-339555), Budesonide (Rhinocort®), C225 (IMC-225, EGFR inhibitor, Anti-EGFr MAb, Cetuximab®), Capecitabine (Xeloda®, Doxifluridine®, oral 5-FU), Carboxyamidotriazole (NSC 609974, CAI, L-651582), CEA-cide® (Labetuzumab®, Anti-CEA monoclonal antibody, hMN-14), Cereport® (Lobradimil®, RMP-7), CI-1033 (Pan-erbB RTK inhibitor), Cilengitide® (EMD-121974, integrin alphavbeta3 antagonist), 9-cis retinoic acid (9-cRA), Cisplatin-liposomal (SPI-077), CMB-401 (Anti-PEM MAb/calicheamycin), CMT-3 (Metastat®), CP-358774 (Tarceva®, OSI-774, EGFR inhibitor), CT-2584 (Apra®), DAB389-EGF (EGF fusion toxin), DeaVac® (CEA anti-idiotype vaccine), Decitabine (5-aza-2′-deoxyytidine), Diethylnorspermine (DENSPM), Dihydro-5-azacytidine, EGF-P64k Vaccine, Endostatin, Etanidazole (Radinyl®), Exetecan mesylate (DX-8951, DX-8951f), Exisulind (SAAND, Aptosyn®, cGMP-PDE2 and 5 inhibitor), FK-317 (FR-157471, FR-70496), Flavopiridol (HMR-1275), Fotemustine (Muphoran®, Mustophoran®), G3139 (Genasense®, GentaAnticode®, Bcl-2 antisense), Gadolinium texaphyrin (Motexafin gadolinium, Gd-Tex®), Xcytrin®), GBC-590, GL331, Galarubicin hydrochloride (DA-125), Glufosfamide® (β-D-glucosyl-isofosfamide mustard, D19575, INN), GVAX (GM-CSF gene therapy), INGN-101 (p53 gene therapy/retrovirus), INGN-201 (p53 gene therapy/adenovirus), Irofulven (MGI-114), ISIS-2053, ISIS-3521 (PKC-alpha antisense), ISIS-5132 (K-ras/raf antisense), Isotretinoin (13-CRA, 13-cis retinoic acid, Accutane®), Lometrexol (T-64, T-904064), Marimastat® (BB-2516, TA-2516, MMP inhibitor), MDX-447 (BAB-447, EMD-82633, H-447, anti-EGFr/FcGammaR1r), MGV, Mitumomab® (BEC-2, EMD-60205), Mivobulin isethionate (CI-980), Neovastat® (AE-941, MMP inhibitor), Onconase (Ranpimase®), Onyx-015 (p53 gene therapy), Pemetrexed disodium (Alimta®, MTA, multitargeted antifolate, LY 231514), Pivaloyloxymethyl butyrate (AN-9, Pivanex®), Prinomastat® (AG-3340, MMP inhibitor), PS-341 (LDP-341, 26S proteasome inhibitor), Pyrazoloacridine (NSC-366140, PD-115934), R115777 (Zamestra®)), Raltitrexed (Tomudex®, ZD-1694), R-flurbiprofen (Flurizan®, E-7869, MPC-7869), RFS-2000 (9-nitrocamptothecan, 9-NC, rubitecan®), RSR-13 (GSJ-61), Satraplatin (BMS-182751, JM-216), SCH-66336, Sizofilan®) (SPG, Sizofuran®, Schizophyllan®, Sonifilan®), Squalamine (MSI-1256F), SR-49059 (vasopressin receptor inhibitor, V1a), SU5416 (Semaxanib®, VEGF inhibitor), Taurolidine (Taurolin®), Temozolamide (Temodar®, NSC 362856), Thalidomide and thalidomide analogs, including but not limited to, lenalidomide (CC-5013, REVLIMID) and CC4047 (ACTIMID), Thymosin alpha I (Zadaxin®, Thymalfasin®), Tirapazamine (SR-259075, SR4233, Tirazone®, Win-59075), TNP470 (AGM-1470), TriAb® (anti-idiotype antibody immune stimulator), Tretinoin (Retin-A®, Atragen®, ATRA, Vesanoid®), Troxacitabine (BCH-204, BCH-4556, Troxatyl®), Vitaxin® (LM-609, integrin alphavbeta3 antagonistic MAb), XR-9576 (P-glycoprotein/MDR inhibitor), and ZD-1839 (IRESSA®).

In preferred embodiments, agonistic antibodies of the invention are administered in combination with one or more of the above-described therapeutic agents in the treatment, amelioration and/or prevention of lung cancers.

In one embodiment, antibodies of the present invention may be administered in combination with a taxane and a platinum-based chemotherapeutic. In another embodiment, antibodies of the present invention may be administered in combination with a taxane and a platinum-based chemotherapeutic for the treatment of lung cancers, such as non-small cell lung cancer, that are resistant to individual chemotherapies. In a specific embodiment, antibodies of the present invention may be administered in combination with Docetaxel (Taxotere®) and Carboplatin (Paraplatin®, CBDCA). In another specific embodiment, antibodies of the present invention may be administered in combination with Docetaxel (Taxotere®) and Carboplatin (Paraplatin®, CBDCA) for the treatment of lung cancers, such as non-small cell lung cancer, that are resistant to individual chemotherapies.

In one embodiment, agonistic antibodies of the present invention may be administered in combination with a taxane and a platinum-based chemotherapeutic. In another embodiment, agonistic antibodies of the present invention may be administered in combination with a taxane and a platinum-based chemotherapeutic for the treatment of lung cancers, such as non-small cell lung cancer, that are resistant to individual chemotherapies. In a specific embodiment, agonistic antibodies of the present invention may be administered in combination with Docetaxel (Taxotere®) and Carboplatin (Paraplatin®, CBDCA). In another specific embodiment, agonistic antibodies of the present invention may be administered in combination with Docetaxel (Taxotere®) and Carboplatin (Paraplatin®, CBDCA) for the treatment of lung cancers, such as non-small cell lung cancer, that are resistant to individual chemotherapies.

Preferred combinations of therapeutic agents useful in the treatment of lung cancer which may be administered in combination with antibodies of the present invention include, but are not limited to, Cisplatin+Docetaxel, Cisplatin+Etoposide, Cisplatin+Gemcitabine, Cisplatin+Interferon alpha, Cisplatin+Irinotecan, Cisplatin+Paclitaxel, Cisplatin+Teniposide, Cisplatin+Vinblastine, Cisplatin+Vindesine, Cisplatin+Vinorelbine, Cisplatin+Vinblastine+Mitomycin C, Cisplatin+Vinorelbine+Gemcitabine, Cisplatin (Platinol®)+Oncovin®+Doxorubicin (Adriamycin®)+Etoposide (CODE), Cyclophosphamide+Adriamycin®+Cisplatin (Platinol®) (CAP), Cyclophosphamide+Adriamycin®+Vincristine (CAV), Cyclophosphamide+Epirubicin+Cisplatin (Platinol®) (CEP), Cyclophosphamide+Methotrexate+Vincristine (CMV), Cyclophosphamide+Adriamycin®, Methotrexate+Fluorouracil (CAMF), Cyclophosphamide+Adriamycin®, Methotrexate+Procarbazine (CAMP), Cyclophosphamide+Adriamycin®, Vincristine+Etoposide (CAV-E), Cyclophosphamide+Adriamycin®, Vincristine+Teniposide (CAV-T), Cyclophosphamide+Oncovin®, Methotrexate+Fluorouracil (COMF), Cyclophosphamide+Adriamycin®+Vincristine, alternating with Cisplatin+Etoposide (CAV/PE), Docetaxel+Gemcitabine, Docetaxel+Vinorelbine, Etoposide (Vepesid®)+Ifosfamide+Cisplatin (Platinol®) (VIP), Etoposide (Vepesid®)+Ifosfamide, Cisplatin+Epirubicin (VIC-E), Fluorouracil+Oncovin®+Mitomycin C (FOMi), Hydrazine+Adriamycin®+Methotrexate (HAM), Ifosfamide+Docetaxel, Ifosfamide+Etoposide, Ifosfamide+Gemcitabine, Ifosfamide+Paclitaxel, Ifosfamide+Vinorelbine, Ifosfamide+Carboplatin+Etoposide (ICE), Irinotecan+Docetaxel, Irinotecan+Etoposide, Irinotecan+Gemcitabine, Methotrexate+Cisplatin, Methotrexate+Interferon alpha, Methotrexate+Vinblastine, Mitomycin C+Ifosfamide+Cisplatin (Platinol®) (MIP), Mitomycin C+Vinblastine+Paraplatin® (MVP), Paraplatin®+Docetaxel, Paraplatin®+Etoposide, Paraplatin®+Gemcitabine, Paraplatin®+Interferon alpha, Paraplatin®+Irinotecan, Paraplatin®+Paclitaxel, Paraplatin®+Vinblastine, Paraplatin®+Vindesine, Paraplatin®+Vinorelbine, Procarbazine+Oncovin®+CCNU® (Lomustine)+Cyclophosphamide (POCC), Vincristine (Oncovin®)+Adriamycin®+Procarbazine (VAP), and Vinorelbine+Gemcitabine.

In preferred embodiments, agonistic antibodies of the invention are administered in combination with one or more of the above-described therapeutic agent combinations in the treatment, amelioration and/or prevention of lung cancers.

In further particular embodiments, antibodies of the present invention are used to treat, ameliorate and/or prevent head and neck cancers including brain cancers. Antibodies of the present invention may be used in combination with one or more surgical and/or radiological procedures and/or therapeutic agents to treat, ameliorate and/or prevent head and neck cancers including brain cancers. Brain cancers which may be treated using antibodies of the present invention include, but are not limited to, gliomas such as astrocytomas and oligodendromas, non-glial tumors such as neuronal, meningeal, ependymal and choroid plexus cell tumors, and metastatic brain tumors such as those originating as breast, lung, prostate and skin cancers.

In further preferred embodiments, agonistic antibodies of the present invention are used to treat, ameliorate and/or prevent head and neck cancers including brain cancers. Agonistic antibodies of the present invention may be used in combination with one or more surgical and/or radiological procedures and/or therapeutic agents to treat, ameliorate and/or prevent head and neck cancers including brain cancers. Brain cancers which may be treated using agonistic antibodies of the present invention include, but are not limited to, gliomas such as astrocytomas and oligodendromas, non-glial tumors such as neuronal, meningeal, ependymal and choroid plexus cell tumors, and metastatic brain tumors such as those originating as breast, lung, prostate and skin cancers.

In one preferred embodiment, agonistic antibodies of the invention are used to treat brain tumors. In a further preferred embodiment, agonistic antibodies of the invention are used to treat glioblastoma multiforme.

Antibodies of the present invention may be administered in combination with one or more radiological procedures useful in the treatment of brain cancers including, but not limited to, external beam radiation therapy, stereotactic radiation therapy, conformal radiation therapy, intensity-modulated radiation therapy (IMRT), and radiosurgery.

In preferred embodiments, agonistic antibodies of the invention are administered in combination with one or more radiological procedures useful in the treatment of brain cancers including, but not limited to, external beam radiation therapy, stereotactic radiation therapy, conformal radiation therapy, intensity-modulated radiation therapy (IT), and radiosurgery.

Antibodies of the present invention may be administered in combination with one or more therapeutic agents useful in the treatment of brain cancers including, but not limited to, Bleomycin (Blenoxane®), Busulfan (Busulfex®, Myleran®), Carboplatin (Paraplatin®, CBDCA), Carmustine (DTI-015, BCNU, BiCNU, Gliadel Wafer®), Cisplatin (Platinol®, CDDP), Cisplatin-epinephrine gel (IntraDose®, FocaCist®), Cyclophosphamide (Cytoxan®, CTX), Cytarabine (Cytosar-UI, ara-C, cytosine arabinoside, DepoCyt®), Dacarbazine (DTIC®), Dactinomycin (Cosmegen®), Daunorubicin (Daunomycin, DaunoXome®, Daunorubicin®, Cerubidine®), Docetaxel (Taxotere®, Taxane®), Dexamethasone (Decadron®), Etoposide phosphate (Etopophos®), Etoposide (VP-16, Vepesid®), Fluorouracil (5-FU, Adrucil®), Hydroxyurea (Hydrea®), Ifosfamide (IFEX®), Lomustine (CCNU®, CeeNU®), Melphalan (L-PAM, Alkeran®, Phenylalanine mustard), Mercaptopurine (6-mercaptopurine, 6-MP), Methchlorethamine (Nitrogen Mustard, HN₂, Mustargen®), Methotrexate® (MTX, Mexate®, Folex®), Paclitaxel (Paxene®, Taxol®), Paclitaxel-DHA (Taxoprexin®), Procarbazine (Matulane®), Temozolamide (Temodar®, NSC 362856), Teniposide (VM-26, Vumon®), Thioguanine (6-thioguanine, 6-TG), Thiotepa (triethylenethiophosphaoramide), Topotecan (Hycamtin®, SK&F-104864, NSC-609699, Evotopin®), and Vincristine (Oncovin®, Onco TCS®, VCR, Leurocristine®).

In preferred embodiments, agonistic antibodies of the invention are administered in combination with one or more of the above-described therapeutic agents in the treatment, amelioration and/or prevention of brain cancers.

Further examples of therapeutic agents useful in the treatment of brain cancers which may be administered in combination with antibodies of the present invention include, but are not limited to, 81C6 (Anti-tenascin monoclonal antibody), BIBX-1382, Cereport® (Lobradimil®, RMP-7), Cilengitide® (EMD-121974, integrin alphavbeta3 antagonist), CMT-3 (Metastat®), Cotara® (chTNT-1/B, [¹³¹I]-chTNT-1/B), CP IL-4-toxin (IL-4 fusion toxin), Fenretinide® (4HPR), Fotemustine (Muphoran®, Mustophoran®), Gemcitabine (Gemto®, Gemzar®), Hypericin® (VIMxyn®), Imatinib mesylate (STI-571, Imatinib®, Glivec®, Gleevec®, Abl tyrosine kinase inhibitor), Irinotecan (Camptosar®, CPT-11, Topotecin®, CaptoCPT-1), Leflunomide (SU-101, SU-0200), Mivobulin isethionate (CI-980), O6-benzylguanine (BG, Procept®), Prinomastat® (AG-3340, MMP inhibitor), R115777 (Zamestra®), SU6668 (PDGF-TK inhibitor), T-67 (T-138067, T-607), Tamoxifen (Nolvadex®), Tf-CRM107 (Transferrin-CRM-107), Thalidomide and thalidomide analogs, including but not limited to, lenalidomide (CC-5013, REVLIMID) and CC-4047 (ACTIMID), Tiazofurin (Tiazole®), Vapreotide® (BMY-41606), Vinorelbine (Navelbine®), and XR-5000 (DACA).

In preferred embodiments, agonistic antibodies of the invention are administered in combination with one or more of the above-described therapeutic agents in the treatment, amelioration and/or prevention of brain cancers.

Preferred combinations of therapeutic agents useful in the treatment of brain cancers which may be administered in combination with antibodies of the present invention include, but are not limited to, Busulfan+Melphalan, Carboplatin+Cereport®, Carboplatin+Etoposide, Carboplatin+Etoposide+Thiotepa, Cisplatin+Etoposide, Cisplatin+Cytarabine+Ifosfamide, Cisplatin+Vincristine+Lomustine, Cisplatin+Cyclophosphamide+Etoposide+Vincristine, Cisplatin+Cytarabine+Ifosfamide+Etoposide+Methotrexate, Cyclophosphamide+Melphalan, Cytarabine+Methotrexate, Dactinomycin+Vincristine, Mechlorethamine+Oncovin® (Vincristine)+Procarbazine (MOP), Mechlorethamine+Oncovin® (Vincristine)+Procarbazine+Prednisone (MOPP), Carboplatin (Paraplatin®)+Etoposide, Carboplatin (Paraplatin®)+Vincristine, Procarbazine+Lomustine, Procarbazine+Lomustine+Vincristine, Procarbazine+Lomustine+Vincristine+Thioguanine, Thiotepa+Etoposide, Thiotepa+Etoposide+Carmustine, Thiotepa+Etoposide+Carboplatin, Vinblastine+Bleomycin+Etoposide+Carboplatin, and Vincristine+Lomustine+Prednisone.

In preferred embodiments, agonistic antibodies of the invention are administered in combination with one or more of the above-described combinations of therapeutic agents in the treatment, amelioration and/or prevention of brain cancers.

In specific embodiments antibodies of the present invention are used to treat, ameliorate and/or prevent skin cancers including basal cell carcinoma, squamous cell carcinoma and malignant melanoma. Antibodies of the present invention may be used in combination with one or more surgical and/or radiological procedures and/or therapeutic agents to treat, ameliorate and/or prevent skin cancers.

In preferred embodiments agonistic antibodies of the present invention are used to treat, ameliorate and/or prevent skin cancers including basal cell carcinoma, squamous cell carcinoma and malignant melanoma. Agonistic antibodies of the present invention may be used in combination with one or more surgical and/or radiological procedures and/or therapeutic agents to treat, ameliorate and/or prevent skin cancers.

Antibodies of the present invention may be administered in combination with one or more therapeutic agents useful in the treatment of skin cancers including, but not limited to, Bleomycin (Blenoxane®), Carmustine (DTI-015, BCNU, BiCNU, Gliadel Wafer®), Cisplatin (Platinol®, CDDP), Dacarbazine (DTIC), Interferon alpha 2b (Intron A®), Interleukin-2 (ProleiukinR®), Tamoxifen (Nolvadex®), Temozolamide (Temodar®, NSC 362856), Vinblastine (Velban®, VLB), Vincristine (Oncovin®, Onco TCS®, VCR, Leurocristine®), and Vindesine (Eldisine®, Fildesin®). Combinations of therapeutic agents useful in the treatment of skin cancers include, but are not limited to, Cisplatin+Carmustine+Dacarbazine+Tamoxifen.

In preferred embodiments, agonistic antibodies of the invention are administered in combination with one or more of the above-described therapeutic agents in the treatment, amelioration and/or prevention of skin cancers.

In further particular embodiments, antibodies of the present invention are used to treat, ameliorate and/or prevent breast cancer. Antibodies of the present invention may be used in combination with one or more surgical and/or radiological procedures and/or therapeutic agents to treat, ameliorate and/or prevent breast cancer. Breast cancers which may be treated using antibodies of the present invention include, but are not limited to, ductal carcinoma, stage I, stage II, stage III and stage IV breast cancers as well as invasive breast cancer and metastatic breast cancer.

In preferred embodiments, agonistic antibodies of the present invention are used to treat, ameliorate and/or prevent breast cancer. Agonistic antibodies of the present invention may be used in combination with one or more surgical and/or radiological procedures and/or therapeutic agents to treat, ameliorate and/or prevent breast cancer. Breast cancers which may be treated using agonistic antibodies of the present invention include, but are not limited to, ductal carcinoma, stage I, stage II, stage III and stage IV breast cancers as well as invasive breast cancer and metastatic breast cancer.

In one preferred embodiment, agonistic antibodies of the invention are used to treat metastatic breast cancer.

Antibodies of the present invention may be administered in combination with one or more surgical and/or radiological procedures useful in the treatment of breast cancer.

In preferred embodiments, agonistic antibodies of the present invention may be administered in combination with one or more surgical and/or radiological procedures useful in the treatment of breast cancer.

Antibodies of the present invention may be administered in combination with one or more therapeutic agents useful in the treatment of breast cancer including, but not limited to, Amifostine (Ethyol®), Aminoglutethimide (Cytadren®), Anastrozole (Arimidex®), Bleomycin (Blenoxane®), Capecitabine (Xeloda®, Doxifluridine®, oral 5-FU), Cisplatin (Platinol®, CDDP), Cisplatin-epinephrine gel (IntraDose®, FocaCist®), Cyclophosphamide (Cytoxan®, Neosar®, CTX), Docetaxel (Taxotere®, Taxane®), Doxorubicin (Adriamycin®1, Doxil®, Rubex®), Epirubicin (Ellence®, EPI, 4′ epi-doxorubicin), Exemestane (Aromasin®, Nikidess®), Fadrozole (Afema®, Fadrozole hydrochloride, Arensin®), Fluorouracil (5-FU, Adrucil®, Fluoroplex®, Efudex®), Herceptin® (Trastuzumab®, Anti-HER-2 monoclonal antibody, Anti-EGFR-2 MAb), Ifosfamide (IFEX®), Letrozole (Femara®), Leucovorin (Leucovorin®, Wellcovorin®), Mechlorethamine (Nitrogen Mustard, HN₂, Mustargen®), Megestrol acetate (Megace®, Pallace®), Melphalan (L-PAM, Alkeran®, Phenylalanine mustard), Methotrexate® (MTX, Mexate®, Folex®), Methyltestosterone (Android-10®, Testred®, Virilon®), Mitomycin C (Mitomycin®, Mutamycin®, Mito Extra®), Orzel® (Tegafur+Uracil+Leucovorin), Paclitaxel (Paxene®, Taxol®)), Sobuzoxane (MST-16, Perazolin®), Tamoxifen (Nolvadex®), Testosterone (Andro®, Androderm®, Testoderm TTS®, Testoderm®, Depo-Testosterone®, Androgel®, depoAndro®), Vinblastine (Velban®, VLB), Vincristine (Oncovin®), Onco TCS®, VCR, Leurocristine®), and Vinorelbine (Navelbine®).

In preferred embodiments, agonistic antibodies of the invention are administered in combination with one or more of the above-described therapeutic agents in the treatment, amelioration and/or prevention of breast cancers.

Further examples of therapeutic agents useful in the treatment of breast cancer which may be administered in combination with antibodies of the present invention include, but are not limited to, Aldesleukin (IL-2, Proleukin®), Altretamine (Hexalen®, hexamethylmelamine, Hexastat®), Angiostatin, Annamycin (AR-522, annamycin LF, Aronex®), Biricodar dicitrate (Incel®, Incel MDR Inhibitor), Boronated Protoporphyrin Compound (PDIT, Photodynamic Immunotherapy), Bryostatin-1 (Bryostatin, BMY45618, NSC-339555), Busulfan (Busulfex®, Myleran®), Carmustine (DTI-015, BCNU, BiCNU, Gliadel Wafer®), D-limonene, Dacarbazine (DTIC), Daunorubicin (Daunomycin, DaunoXome®, Daunorubicin®, Cerubidine®), Dolastatin-10 (DOLA-10, NSC-376128), DPPE, DX-8951f (DX-8951), EMD-121974, Endostatin, EO9 (EO1, EO4, EO68, EO70, EO72), Etoposide phosphate (Etopophos®), Etoposide (VP-16, Vepesid®), Fluasterone, Fludarabine (Fludara®, FAMP), Flutamide (Eulexin®), Formestane (Lentaron®), Fulvestrant (Faslodex®), Galarubicin hydrochloride (DA-125), Gemcitabine (Gemto®, Gemzar®), Her-2/Neu vaccine, Hydroxyurea (Hydrea®), Idarubicin (Idamycin®, DMDR, IDA), Interferon alpha 2a (Intron A®), Interferon gamma (Gamma-interferon, Gamma 1000, Gamma-1F), Irinotecan (Camptosar®, CPT-11, Topotecin®, CaptoCPT-1), Ketoconazole (Nizoral®), KRN-8602 (MX, MY-5, NSC-619003, MX-2), L-asparaginase (Elspar®), Leuprolide acetate (Viadur®, Lupron®), Lomustine (CCNU®, CeeNU®), LY-335979, Mannan-MUC1 vaccine, 2-Methoxyestradiol (2-ME, 2-ME2), Mitoxantrone (Novantrone®, DHAD), Motexafin Lutetium (Lutrin®, Optrin®, Lu-Tex®, lutetium texaphyrin, Lucyn®, Antrin®), MPV-2213ad (Finrozole®), MS-209, Muc-1 vaccine, NaPro Paclitaxel, Perillyl alcohol (perilla alcohol, perillic alcohol, perillol, NSC-641066), Pirarubicin (THP), Procarbazine (Matulane®), Providence Portland Medical Center Breast Cancer Vaccine, Pyrazoloacridine (NSC-366140, PD-115934), Raloxifene hydrochloride (Evista®, Keoxifene hydrochloride), Raltitrexed (Tomudex®, ZD-1694), Rebeccamycin, Streptozocin (Zanosar®), Temozolamide (Temodar®, NSC 362856), Theratope, Thiotepa (triethylenethiophosphaoramide, Thioplex®), Topotecan (Hycamtin®, SK&F-104864, NSC-609699, Evotopin®), Toremifene (Estrimex®, Fareston®), Trilostane (Modrefen®), and XR-9576 (XR-9351, P-glycoprotein/MDR inhibitor).

In preferred embodiments, agonistic antibodies of the invention are administered in combination with one or more of the above-described therapeutic agents in the treatment, amelioration and/or prevention of breast cancers.

Preferred combinations of therapeutic agents useful in the treatment of breast cancer which may be administered in combination with antibodies of the present invention include, but are not limited to, Cyclophosphamide+Adriamycin® (Doxorubicin), Cyclophosphamide+Epirubicin+Fluorouracil, Cyclophosphamide+Methotrexate+Fluorouracil (CMF), Paclitaxel+Doxorubicin, and Vinblastine+Doxorubicin+Thiotepa.

In preferred embodiments, agonistic antibodies of the invention are administered in combination with one or more of the above-described therapeutic agent combinations in the treatment, amelioration and/or prevention of breast cancers.

In further particular embodiments, antibodies of the present invention are used to treat, ameliorate and/or prevent prostate cancer. Antibodies of the present invention may be used in combination with one or more surgical and/or radiological procedures and/or therapeutic agents to treat, ameliorate and/or prevent prostate cancer. Prostate cancer which may be treated using antibodies of the present invention includes, but is not limited to, benign prostatic hyperplasia, malignant prostate cancer (e.g., stage I, stage II, stage III or stage IV) and metastatic prostate cancer.

In preferred embodiments, agonistic antibodies of the present invention are used to treat, ameliorate and/or prevent prostate cancer. Agonistic antibodies of the present invention may be used in combination with one or more surgical and/or radiological procedures and/or therapeutic agents to treat, ameliorate and/or prevent prostate cancer. Prostate cancer which may be treated using agonistic antibodies of the present invention includes, but is not limited to, benign prostatic hyperplasia, malignant prostate cancer (e.g., stage I, stage II, stage III or stage IV) and metastatic prostate cancer.

In one preferred embodiment, agonistic antibodies of the invention are used to treat malignant prostate cancer. In a further preferred embodiment, agonistic antibodies of the invention are used to treat metastatic prostate cancer.

Antibodies of the present invention may be administered in combination with one or more surgical, radiological and/or hormonal procedures useful in the treatment of prostate cancer including, but not limited to, prostatectomy (e.g., radical retropubic prostatectomy), external beam radiation therapy, brachytherapy, orchiectomy and hormone treatment (e.g., LHRH agonists, androgen receptor inhibitors).

In preferred embodiments, agonistic antibodies of the present invention may be administered in combination with one or more surgical, radiological and/or hormonal procedures useful in the treatment of prostate cancer including, but not limited to, prostatectomy (e.g., radical retropubic prostatectomy), external beam radiation therapy, brachytherapy, orchiectomy and hormone treatment (e.g., LHRH agonists, androgen receptor inhibitors).

Antibodies of the present invention may be administered in combination with one or more therapeutic agents useful in the treatment of prostate cancer including, but not limited to, Aminoglutethimide (Cytadren®), Biclutamide (Casodex®), Cyclophosphamide (Cytoxan®, Neosar®, CTX), Diethylstilbestrol (DES), Doxorubicin (Adriamycin®, Doxil®, Rubex®), Flutamide (Eulexin®)), Hydrocortisone, Ketoconazole (Nizoral®), Leuprolide acetate (Viadur®, Lupron®, Leuprogel®, Eligard®), Mitoxantrone (Novantrone®, DHAD), Nilutamide (Nilandron®), Paclitaxel (Paxene®, Taxol®), Paclitaxel-DHA (Taxoprexin®), PC SPES, Prednisone, Triptorelin pamoate (Trelstar Depot®, Decapeptyl®)), and Vinblastine (Velban®, VLB).

In preferred embodiments, agonistic antibodies of the invention are administered in combination with one or more of the above-described therapeutic agents in the treatment, amelioration and/or prevention of prostate cancers.

Further examples of therapeutic agents useful in the treatment of prostate cancer which may be administered in combination with antibodies of the present invention include, but are not limited to, Abarelix® (Abarelix-Depot-Mg, PPI-149, R-3827); Abiraterone acetate® (CB-7598, CB-7630), ABT-627 (ET-1 inhibitor), APC-8015 (Provenge®, Dendritic cell therapy), Avorelin® (Meterelin®, MF-6001, EP-23904), CEP-701 (KT-5555), CN-706, CT-2584 (Apra®, CT-2583, CT-2586, CT-3536), GBC-590, Globo H hexasaccharide (Globo H-KLH®), Interferon alpha 2a (Intron A®), Liarozole (Liazal, Liazol, R-75251, R-85246, Ro-85264), MDX-447 (MDX-220, BAB-447, EMD-82633, H-447, anti-EGFr/FcGammaR1r), OncoVAX-P (OncoVAX-PrPSA), PROSTVAC, PS-341 (LDP-341, 26S proteasome inhibitor), PSMA MAb (Prostate Specific Membrane Antigen monoclonal antibody), and R-flurbiprofen (Flurizan®, E-7869, MPC-7869).

In preferred embodiments, agonistic antibodies of the invention are administered in combination with one or more of the above-described therapeutic agents in the treatment, amelioration and/or prevention of prostate cancers.

Preferred combinations of therapeutic agents useful in the treatment of prostate cancer which may be administered in combination with antibodies of the present invention include, but are not limited to, Docetaxel+Estramustine, Mitoxantrone+Hydrocortisone, Mitoxantrone+Prednisone, Navelbine+Estramustine, Paclitaxel+Estramustine, and Vinblastine+Estramustine.

In preferred embodiments, agonistic antibodies of the invention are administered in combination with one or more of the above-described therapeutic agent combinations in the treatment, amelioration and/or prevention of prostate cancers.

In further particular embodiments, antibodies of the present invention are used to treat, ameliorate and/or prevent pancreatic cancer. Antibodies of the present invention may be used in combination with one or more surgical and/or radiological procedures and/or therapeutic agents to treat, ameliorate and/or prevent pancreatic cancer. Pancreatic cancers which may be treated using antibodies of the present invention include, but are not limited to, adenocarcinoma, endocrine (islet cell) tumors, tumors confined to the pancreas, locally advanced pancreatic cancer and metastatic pancreatic cancer.

In preferred embodiments, agonistic antibodies of the present invention are used to treat, ameliorate and/or prevent pancreatic cancer. Agonistic antibodies of the present invention may be used in combination with one or more surgical and/or radiological procedures and/or therapeutic agents to treat, ameliorate and/or prevent pancreatic cancer. Pancreatic cancers which may be treated using agonistic antibodies of the present invention include, but are not limited to, adenocarcinoma, endocrine (islet cell) tumors, tumors confined to the pancreas, locally advanced pancreatic cancer and metastatic pancreatic cancer.

In one preferred embodiment, agonistic antibodies of the invention are used to treat locally advanced pancreatic cancer. In a further preferred embodiment, agonistic antibodies of the invention are used to treat metastatic pancreatic cancer.

Antibodies of the present invention may be administered in combination with one or more surgical and/or radiological procedures useful in the treatment of pancreatic cancer including, but not limited to, pancreaticoduodenumectomy (Whipple resection).

In preferred embodiments, agonistic antibodies of the present invention may be administered in combination with one or more surgical and/or radiological procedures useful in the treatment of pancreatic cancer including, but not limited to, pancreaticoduodenumectomy (Whipple resection).

Antibodies of the present invention may be administered in combination with one or more therapeutic agents useful in the treatment of pancreatic cancer including, but not limited to, Capecitabine (Xeloda®, Doxifluridine®, oral 5-FU), Cisplatin (Platinol®, CDDP), Fluorouracil (5-FU, Adrucil®, Fluoroplex®, Efudex®), Gemcitabine (Gemto®, Gemzar®), and Irinotecan (Camptosar®, CPT-11, Topotecin®, CaptoCPT-1).

In preferred embodiments, agonistic antibodies of the invention are administered in combination with one or more of the above-described therapeutic agents in the treatment, amelioration and/or prevention of pancreatic cancers.

Preferred combinations of therapeutic agents useful in the treatment of pancreatic cancer which may be administered in combination with antibodies of the present invention include, but are not limited to, Cisplatin+Gemcitabine, CP-358774+Gemcitabine, Docetaxel+Gemcitabine, Irinotecan+Fluorouracil, Irinotecan+Gemcitabine, and Paclitaxel+Gemcitabine.

In preferred embodiments, agonistic antibodies of the invention are administered in combination with one or more of the above-described therapeutic agent combinations in the treatment, amelioration and/or prevention of pancreatic cancers.

Further examples of therapeutic agents useful in the treatment of pancreatic cancer which may be administered in combination with antibodies of the present invention include, but are not limited to, ABX-EGF (anti-EGFr MAb), Acetyldinaline (CI-994, GOE-5549, GOR-5549, PD-130636), BMS-214662 (BMS-192331, BMS-193269, BMS-206635), BNP-1350 (BNPI-1100, Karenitecins), C225 (IMC-225, EGFR inhibitor, Anti-EGFr MAb, Cetuximab®), C242-DMI (huC242-DM1, SB-408075), Carbendazin® (FB-642), Carmustine (DTI-015, BCNU, BiCNU, Gliadel Wafer®), CMT-3 (COL-3, Metastat®), CP-358774 (Tarceva®, OSI-774, EGFR inhibitor), Docetaxel (Taxotere®, Taxane®), Exetecan mesylate (DX-8951, DX-8951f), Flavopiridol (HMR-1275), Gastrimmune® (Anti-gastrin-17 immunogen, anti-g17), GBC-590, Herceptin® (Trastuzumab®, Anti-HER-2 monoclonal antibody, Anti-EGFR-2 MAb), HSPPC-96 (HSP cancer vaccine, gp96 heat shock protein-peptide complex), Irofulven (MGI-114), ISIS-2503 (Ras antisense), Onyx-015 (p53 gene therapy), Paclitaxel (Paxene®, Taxol®), Pemetrexed disodium (Alimta®, MTA, multitargeted antifolate, LY 231514), Perillyl alcohol (perilla alcohol, perillic alcohol, perillol, NSC-641066), RFS-2000 (9-nitrocamptothecan, 9-NC, rubitecan®), and Rituximab® (Rituxan®, anti-CD20 MAb).

In preferred embodiments, agonistic antibodies of the invention are administered in combination with one or more of the above-described therapeutic agents in the treatment, amelioration and/or prevention of pancreatic cancers.

In further particular embodiments, antibodies of the present invention are used to treat, ameliorate and/or prevent hepatic cancer. Antibodies of the present invention may be used in combination with one or more surgical and/or radiological procedures and/or therapeutic agents to treat, ameliorate and/or prevent hepatic cancer. Hepatic cancers which may be treated using antibodies of the present invention include, but are not limited to, hepatocellular carcinoma, malignant hepatoma, cholangiocarcinoma, mixed hepatocellular cholangiocarcinoma or hepatoblastoma.

In preferred embodiments, agonistic antibodies of the present invention are used to treat, ameliorate and/or prevent hepatic cancer. Agonistic antibodies of the present invention may be used in combination with one or more surgical and/or radiological procedures and/or therapeutic agents to treat, ameliorate and/or prevent hepatic cancer. Hepatic cancers which may be treated using agonistic antibodies of the present invention include, but are not limited to, hepatocellular carcinoma, malignant hepatoma, cholangiocarcinoma, mixed hepatocellular cholangiocarcinoma or hepatoblastoma.

In one preferred embodiment, agonistic antibodies of the invention are used to treat hepatoblastoma. In one further preferred embodiment, agonistic antibodies of the invention are used to treat hepatocellular carcinoma.

Antibodies of the present invention may be administered in combination with one or more surgical and/or radiological procedures useful in the treatment of hepatic cancers including, but not limited to, partial hepatectomy, liver transplant, radiofrequency ablation, laser therapy, microwave therapy, cryosurgery, percutaneous ethanol injection, hepatic arterial infusion, hepatic artery ligation, chemoembolization and external beam radiation therapy.

In preferred embodiments, agonistic antibodies of the present invention may be administered in combination with one or more surgical and/or radiological procedures useful in the treatment of hepatic cancers including, but not limited to, partial hepatectomy, liver transplant, radiofrequency ablation, laser therapy, microwave therapy, cryosurgery, percutaneous ethanol injection, hepatic arterial infusion, hepatic artery ligation, chemoembolization and external beam radiation therapy.

Antibodies of the present invention may be administered in combination with one or more therapeutic agents useful in the treatment of hepatic cancer including, but not limited to, Aldesleukin (IL-2, Proleukin®), Cisplatin (Platinol®, CDDP), Doxorubicin (Adriamycin®, Doxil®, Rubex®), Etoposide phosphate (Etopophos®), Etoposide (VP-16, Vepesid®), Fluorouracil (5-FU, Adrucil®, Fluoroplex®, Efudex®), I-131 Lipidiol®, Ifosfamide (IFEX®), Megestrol acetate (Megace®, Pallace®), Pravastatin sodium (Pravachol®), and Vincristine (Oncovin®, Onco TCS®, VCR, Leurocristine®).

In preferred embodiments, agonistic antibodies of the invention are administered in combination with one or more of the above-described therapeutic agents in the treatment, amelioration and/or prevention of hepatic cancers.

Preferred combinations of therapeutic agents useful in the treatment of hepatic cancer which may be administered in combination with antibodies of the present invention include, but are not limited to, Cisplatin+Doxorubicin, Cisplatin+Etoposide, Cisplatin+Vincristine+Fluorouracil, and Ifosfamide+Cisplatin+Doxorubicin.

In preferred embodiments, agonistic antibodies of the invention are administered in combination with one or more of the above-described therapeutic agent combinations in the treatment, amelioration and/or prevention of hepatic cancers.

In further particular embodiments, antibodies of the present invention are used to treat, ameliorate and/or prevent ovarian cancer. Antibodies of the present invention may be used in combination with one or more surgical and/or radiological procedures and/or therapeutic agents to treat, ameliorate and/or prevent ovarian cancer. Ovarian cancers which may be treated using antibodies of the present invention include, but are not limited to, epithelial carcinoma, germ cell tumors and stromal tumors.

In preferred embodiments, agonistic antibodies of the present invention are used to treat, ameliorate and/or prevent ovarian cancer. Agonistic antibodies of the present invention may be used in combination with one or more surgical and/or radiological procedures and/or therapeutic agents to treat, ameliorate and/or prevent ovarian cancer. Ovarian cancers which may be treated using agonistic antibodies of the present invention include, but are not limited to, epithelial carcinoma, germ cell tumors and stromal tumors.

In one preferred embodiment, agonistic antibodies of the invention are used to treat germ cell tumors. In one further preferred embodiment, agonistic antibodies of the invention are used to treat epithelial carcinoma.

Antibodies of the present invention may be administered in combination with one or more surgical and/or radiological procedures useful in the treatment of ovarian cancer including, but not limited to, hysterectomy, oophorectomy, hysterectomy with bilateral salpingo-oophorectomy, omentectomy, tumor debulking, external beam radiation therapy and intraperitoneal radiation therapy.

In preferred embodiments, agonistic antibodies of the present invention may be administered in combination with one or more surgical and/or radiological procedures useful in the treatment of ovarian cancer including, but not limited to, hysterectomy, oophorectomy, hysterectomy with bilateral salpingo-oophorectomy, omentectomy, tumor debulking, external beam radiation therapy and intraperitoneal radiation therapy.

Antibodies of the present invention may be administered in combination with one or more therapeutic agents useful in the treatment of ovarian cancer including, but not limited to, Altretamine (Hexalen®, hexamethylmelamine, Hexastat®), Bleomycin (Blenoxane®), Carboplatin (Paraplatin®, CBDCA), Cisplatin (Platinol®, CDDP), Cyclophosphamide (Cytoxan®, Neosar®, CTX), Dactinomycin (Cosmegen®), Doxorubicin (Adriamycin®, Doxil®, Rubex®), Etoposide phosphate (Etopophos®), Etoposide (VP-16, Vepesid®), Fluorouracil (5-FU, Adrucil®, Fluoroplex®, Efudex®), Gemcitabine (Gemto®, Gemzar®), Ifosfamide (IFEX®), Irinotecan (Camptosar®, CPT-11, Topotecin®, CaptoCPT-1), Leucovorin (Leucovorin®, Welicovorin®), Melphalan (L-PAM, Alkeran®, Phenylalanine mustard), Paclitaxel (Paxene®, Taxol®), Tamoxifen (Nolvadex®), Vinblastine (Velban®, VLB) and Vincristine (Oncovin®, Onco TCS®, VCR, Leurocristine®).

In preferred embodiments, agonistic antibodies of the invention are administered in combination with one or more of the above-described therapeutic agents in the treatment, amelioration and/or prevention of ovcarian cancers.

Preferred combinations of therapeutic agents useful in the treatment of ovarian cancer which may be administered in combination with antibodies of the present invention include, but are not limited to, Bleomycin+Etoposide+Platinol® (Cisplatin) (BEP), Carboplatin+Cyclophosphamide, Carboplatin+Paclitaxel, Carboplatin+Etoposide+Bleomycin (CEB), Cisplatin+Cyclophosphamide, Cisplatin+Etoposide, Cisplatin+Paclitaxel, Cisplatin+Ifosfamide+Vinblastine, Fluorouracil+Leucovorin, Platinol® (Cisplatin)+Vinblastine+Bleomycin (PVB), and Vincristine+Dactinomycin+Cyclophosphamide.

In preferred embodiments, agonistic antibodies of the invention are administered in combination with one or more of the above-described therapeutic agent combinations in the treatment, amelioration and/or prevention of ovarian cancers.

In further particular embodiments, antibodies of the present invention are used to treat, ameliorate and/or prevent Ewing's sarcoma. Antibodies of the present invention may be used in combination with one or more surgical and/or radiological procedures and/or therapeutic agents to treat, ameliorate and/or prevent Ewing's sarcoma. Ewing's sarcoma family tumors which may be treated using antibodies of the present invention include, but are not limited to, Ewing's tumor of bone (ETB), extraosseus Ewing's (EOE), primitive neuroectodermal tumors (PNET or peripheral neuroepithelioma) and Askin's tumor.

In preferred embodiments, agonistic antibodies of the present invention are used to treat, ameliorate and/or prevent Ewing's sarcoma. Agonistic antibodies of the present invention may be used in combination with one or more surgical and/or radiological procedures and/or therapeutic agents to treat, ameliorate and/or prevent Ewing's sarcoma. Ewing's sarcoma family tumors which may be treated using agonistic antibodies of the present invention include, but are not limited to, Ewing's tumor of bone (ETB), extraosseus Ewing's (EOE), primitive neuroectodermal tumors (PNET or peripheral neuroepithelioma) and Askin's tumor.

In one preferred embodiment, agonistic antibodies of the invention are used to treat Ewing's tumor of bone. In one further preferred embodiment, agonistic antibodies of the invention are used to treat peripheral neuroepithelioma.

Antibodies of the present invention may be administered in combination with one or more surgical and/or radiological procedures useful in the treatment of Ewing's sarcoma family tumors.

In preferred embodiments, agonistic antibodies of the invention are administered in combination with one or more surgical and/or radiological procedures useful in the treatment of Ewing's sarcoma family tumors.

Antibodies of the present invention may be administered in combination with one or more therapeutic agents useful in the treatment of Ewing's sarcoma family tumors including, but not limited to, Cyclophosphamide (Cytoxan®, Neosar®, CTX), Doxorubicin (Adriamycin®, Doxil®, Rubex®), Etoposide phosphate (Etopophos®), Etoposide (VP-16, Vepesid®), Filgrastim (Neupogen®, G-CSF), Ifosfamide (IFEX®), Topotecan (Hycamtin®, SK&F-104864, NSC-609699, Evotopin®), and Vincristine (Oncovin®, Onco TCS®, VCR, Leurocristine®).

In preferred embodiments, agonistic antibodies of the invention are administered in combination with one or more of the above-described therapeutic agents in the treatment, amelioration and/or prevention of Ewing's sarcoma family tumors.

Preferred combinations of therapeutic agents useful in the treatment of Ewing's sarcoma family tumors which may be administered in combination with antibodies of the present invention include, but are not limited to, Cyclophosphamide+Topotecan, Cyclophosphamide+Doxorubicin+Vincristine, Cyclophosphamide+Doxorubicin+Vincristine, alternating with Ifosfamide+Etoposide andcyclophosphamide+Doxorubicin+Vincristine, alternating with Filgrastim+Ifosfamide+Etoposide.

In preferred embodiments, agonistic antibodies of the invention are administered in combination with one or more of the above-described therapeutic agent combinations in the treatment, amelioration and/or prevention of Ewing's sarcoma family tumors.

Additional Combination Therapies

In a more preferred embodiment, the antibody and antibody compositions of the invention are administered in combination with an antimalarial, methotrexate, anti-TNF antibody, ENBREL™ and/or suflasalazine. In one embodiment, the antibody and antibody compositions of the invention are administered in combination with methotrexate. In another embodiment, the antibody and antibody compositions of the invention are administered in combination with anti-TNF antibody. In another embodiment, the antibody and antibody compositions of the invention are administered in combination with methotrexate and anti-TNF antibody. In another embodiment, the antibody and antibody compositions of the invention are administered in combination with suflasalazine. In another specific embodiment, the antibody and antibody compositions of the invention are administered in combination with methotrexate, anti-TNF antibody, and suflasalazine. In another embodiment, the antibody and antibody compositions of the invention are administered in combination ENBREL™. In another embodiment, the antibody and antibody compositions of the invention are administered in combination with ENBREL™ and methotrexate. In another embodiment, the antibody and antibody compositions of the invention are administered in combination with ENBREL™, methotrexate and suflasalazine. In another embodiment, the antibody and antibody compositions of the invention are administered in combination with ENBREL™, methotrexate and suflasalazine. In other embodiments, one or more antimalarials is combined with one of the above-recited combinations. In a specfic embodiment, the antibody and antibody compositions of the invention are administered in combination with an antimalarial (e.g., hydroxychloroquine), ENBREL™, methotrexate and suflasalazine. In another specfic embodiment, the antibody and antibody compositions of the invention are administered in combination with an antimalarial (e.g., hydroxychloroquine), sulfasalazine, anti-TNF antibody, and methotrexate.

The antibodies of the invention (including molecules comprising, or alternatively consisting of, antibody fragments or variants thereof) may be administered alone or in combination with other therapeutic or prophylactic regimens (e.g., radiation therapy, chemotherapy, hormonal therapy, immunotherapy, anti-tumor agents, anti-angiogenesis and anti-inflammatory agents). Such combinatorial therapy may be administered sequentially and/or concomitantly.

Conventional nonspecific immunosuppressive agents, that may be administered in combination with the antibody and antibody compositions of the invention include, but are not limited to, steroids, cyclosporine, cyclosporine analogs cyclophosphamide, cyclophosphamide IV, methylprednisolone, prednisolone, azathioprine, FK-506, 15-deoxyspergualin, and other immunosuppressive agents that act by suppressing the function of responding T cells.

In specific embodiments, antibody and antibody compositions of the invention are administered in combination with immunosuppressants. Immunosuppressants preparations that may be administered with the antibody and antibody compositions of the invention include, but are not limited to, ORTHOCLONE™ (OKT3), SAND T m/NE ORAL™/SANGDYA™ (cyclosporin), PROGRAF™ (tacrolimus), CELLCEP™ (mycophenolate), Azathioprine, glucorticosteroids, and RAPAMUNE™ (sirolimus). In a specific embodiment, immunosuppressants may be used to prevent rejection of organ or bone marrow transplantation.

In a preferred embodiment, the antibody and antibody compositions of the invention are administered in combination with steroid therapy. Steroids that may be administered in combination with the antibody and antibody compositions of the invention, include, but are not limited to, oral corticosteroids, prednisone, and methylprednisolone (e.g., IV methylprednisolone). In a specific embodiment, antibody and antibody compositions of the invention are administered in combination with prednisone. In a further specific embodiment, the antibody and antibody compositions of the invention are administered in combination with prednisone and an immunosuppressive agent. Immunosuppressive agents that may be administered with the antibody and antibody compositions of the invention and prednisone are those described herein, and include, but are not limited to, azathioprine, cylophosphamide, and cyclophosphamide IV. In another specific embodiment, antibody and antibody compositions of the invention are administered in combination with methylprednisolone. In a further specific embodiment, the antibody and antibody compositions of the invention are administered in combination with methylprednisolone and an immunosuppressive agent. Immunosuppressive agents that may be administered with the antibody and antibody compositions of the invention and methylprednisolone are those described herein, and include, but are not limited to, azathioprine, cylophosphamide, and cyclophosphamide IV.

The invention also encompasses combining the polynucleotides and/or polypeptides of the invention (and/or agonists or antagonists thereof) with other proposed or conventional hematopoietic therapies. Thus, for example, the polynucleotides and/or polypeptides of the invention (and/or agonists or antagonists thereof) can be combined with compounds that singly exhibit erythropoietic stimulatory effects, such as erythropoietin, testosterone, progenitor cell stimulators, insulin-like growth factor, prostaglandins, serotonin, cyclic AMP, prolactin, and triiodothyzonine. Also encompassed are combinations of the antibody and antibody compositions of the invention with compounds generally used to treat aplastic anemia, such as, for example, methenolene, stanozolol, and nandrolone; to treat iron-deficiency anemia, such as, for example, iron preparations; to treat malignant anemia, such as, for example, vitamin B₁₂ and/or folic acid; and to treat hemolytic anemia, such as, for example, adrenocortical steroids, e.g., corticoids. See e.g., Resegotti et al., Panminerva Medica, 23:243-248 (1981); Kurtz, FEBS Letters, 14a: 105-108 (1982); McGonigle et al., Kidney Int., 25:437-444 (1984); and Pavlovic-Kantera, Expt. Hematol., 8(supp. 8) 283-291 (1980), the contents of each of which are hereby incorporated by reference in their entireties.

Compounds that enhance the effects of or synergize with erythropoietin are also useful as adjuvants herein, and include but are not limited to, adrenergic agonists, thyroid hormones, androgens, hepatic erythropoietic factors, erythrotropins, and erythrogenins, See for e.g., Dunn, “Current Concepts in Erythropoiesis”, John Wiley and Sons (Chichester, England, 1983); Kalmani, Kidney Int., 22:383-391 (1982); Shahidi, New Eng. J. Med., 289:72-80 (1973); Urabe et al., J. Exp. Med., 149:1314-1325 (1979); Billat et al., Expt. Hematol., 10:135-140 (1982); Naughton et al., Acta Haemat, 69:171-179 (1983); Cognote et al. in abstract 364, Proceedings 7th Intl. Cong. of Endocrinology (Quebec City, Quebec, Jul. 1-7, 1984); and Rothman et al., 1982, J. Surg. Oncol., 20:105-108 (1982). Methods for stimulating hematopoiesis comprise administering a hematopoietically effective amount (i.e., an amount which effects the formation of blood cells) of a pharmaceutical composition containing polynucleotides and/or poylpeptides of the invention (and/or agonists or antagonists thereof) to a patient. The polynucleotides and/or polypeptides of the invention and/or agonists or antagonists thereof is administered to the patient by any suitable technique, including but not limited to, parenteral, sublingual, topical, intrapulmonary and intranasal, and those techniques further discussed herein. The pharmaceutical composition optionally contains one or more members of the group consisting of erythropoietin, testosterone, progenitor cell stimulators, insulin-like growth factor, prostaglandins, serotonin, cyclic AMP, prolactin, triiodothyzonine, methenolene, stanozolol, and nandrolone, iron preparations, vitamin B₁₂, folic acid and/or adrenocortical steroids.

In an additional embodiment, the antibody and antibody compositions of the invention are administered in combination with hematopoietic growth factors. Hematopoietic growth factors that may be administered with the antibody and antibody compositions of the invention include, but are not limited to, LEUKINE™ (SARGRAMOSTIM™) and NEUPOGEN™ (FILGRASTIM™).

In an additional embodiment, the antibody and antibody compositions of the invention are administered alone or in combination with an anti-angiogenic agent(s). Anti-angiogenic agents that may be administered with the antibody and antibody compositions of the invention include, but are not limited to, Angiostatin (Entremed, Rockville, Md.), Troponin-1 (Boston Life Sciences, Boston, Mass.), anti-Invasive Factor, retinoic acid and derivatives thereof, paclitaxel (Taxol), Suramin, Tissue Inhibitor of Metalloproteinase-1, Tissue Inhibitor of Metalloproteinase-2, VEGI, Plasminogen Activator Inhibitor-1, Plasminogen Activator Inhibitor-2, and various forms of the lighter “d group” transition metals.

Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate and orthovanadate complexes such as, for example, ammonium metavanadate, sodium metavanadate, and sodium orthovanadate. Suitable vanadyl complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate complexes include ammonium tungstate, calcium tungstate, sodium tungstate dihydrate, and tungstic acid. Suitable tungsten oxides include tungsten (IV) oxide and tungsten (VI) oxide. Suitable oxo molybdenum complexes include molybdate, molybdenum oxide, and molybdenyl complexes. Suitable molybdate complexes include ammonium molybdate and its hydrates, sodium molybdate and its hydrates, and potassium molybdate and its hydrates. Suitable molybdenum oxides include molybdenum (VI) oxide, molybdenum (VI) oxide, and molybdic acid. Suitable molybdenyl complexes include, for example, molybdenyl acetylacetonate. Other suitable tungsten and molybdenum complexes include hydroxo derivatives derived from, for example, glycerol, tartaric acid, and sugars.

A wide variety of other anti-angiogenic factors may also be utilized within the context of the present invention. Representative examples include, but are not limited to, platelet factor 4; protamine sulphate; sulphated chitin derivatives (prepared from queen crab shells), (Murata et al., Cancer Res. 51:22-26, 1991); Sulphated Polysaccharide Peptidoglycan Complex (SP-PG) (the function of this compound may be enhanced by the presence of steroids such as estrogen, and tamoxifen citrate); Staurosporine; modulators of matrix metabolism, including for example, proline analogs, cishydroxyproline, d,L-3,4-dehydroproline, Thiaproline, alpha,alpha-dipyridyl, aminopropionitrile fumarate; 4-propyl-5-(4-pyridinyl)-2(3H)-oxazolone; Methotrexate; Mitoxantrone; Heparin; Interferons; 2 Macroglobulin-serum; ChIMP-3 (Pavloff et al., J. Biol. Chem. 267:17321-17326, 1992); Chymostatin (Tomkinson et al., Biochem J. 286:475-480, 1992); Cyclodextrin Tetradecasulfate; Eponemycin; Camptothecin; Fumagillin (Ingber et al., Nature 348:555-557, 1990); Gold Sodium Thiomalate (“GST”; Matsubara and Ziff, J. Clin. Invest. 79:1440-1446, 1987); anticollagenase-serum; alpha2-antiplasmin (Holmes et al., J. Biol. Chem. 262(4):1659-1664, 1987); Bisantrene (National Cancer Institute); Lobenzarit disodium (N-2)-carboxyphenyl-4-chloroanthronilic acid disodium or “CCA”; (Takeuchi et al., Agents Actions 36:312-316, 1992); and metalloproteinase inhibitors such as BB94.

Additional anti-angiogenic factors that may also be utilized within the context of the present invention include Thalidomide, (Celgene, Warren, N.J.) and thalidomide analogs, including but not limited to, lenalidomide (CC-5013, REVLIMID) and CC4047 (ACTIMID); Angiostatic steroid; AGM-1470 (H. Brem and J. Folkman J Pediatr. Surg. 28:445-51 (1993)); an integrin alpha v beta 3 antagonist (C. Storgard et al., J. Clin. Invest. 103:47-54 (1999)); carboxynaminolmidazole; Carboxyamidotriazole (CAI) (National Cancer Institute, Bethesda, Md.); Conbretastatin A-4 (CA4P) (OXiGENE, Boston, Mass.); Squalamine (Magainin Pharmaceuticals, Plymouth Meeting, Pa.); TNP470, (Tap Pharmaceuticals, Deerfield, Ill.); ZD-0101 AstraZeneca (London, UK); APRA (CT2584); Benefin, Byrostatin-1 (SC359555); CGP41251 (PKC 412); CM101; Dexrazoxane (ICRF187); DMXAA; Endostatin; Flavopridiol; Genestein; GTE; ImmTher; Iressa (ZD1839); Octreotide (Somatostatin); Panretin; Penacillamine; Photopoint; PI-88; Prinomastat (AG-3540) Purlytin; Suradista (FCE26644); Tamoxifen (Nolvadex); Tazarotene; Tetrathiomolybdate; Xeloda (Capecitabine); and 5-Fluorouracil.

Anti-angiogenic agents that may be administered in combination with the compounds of the invention may work through a variety of mechanisms including, but not limited to, inhibiting proteolysis of the extracellular matrix, blocking the function of endothelial cell-extracellular matrix adhesion molecules, by antagonizing the function of angiogenesis inducers such as growth factors, and inhibiting integrin receptors expressed on proliferating endothelial cells. Examples of anti-angiogenic inhibitors that interfere with extracellular matrix proteolysis and which may be administered in combination with the antibody and antibody compositions of the invention include, but are not limited to, AG-3540 (Agouron, La Jolla, Calif.), BAY-12-9566 (Bayer, West Haven, Conn.), BMS-275291 (Bristol Myers Squibb, Princeton, N.J.), CGS-27032A (Novartis, East Hanover, N.J.), Marimastat (British Biotech, Oxford, UK), and Metastat (Aetema, St-Foy, Quebec). Examples of anti-angiogenic inhibitors that act by blocking the function of endothelial cell-extracellular matrix adhesion molecules and which may be administered in combination with the antibody and antibody compositions of the invention include, but are not limited to, EMD-121974 (Merck KcgaA Darmstadt, Germany) and Vitaxin (Ixsys, La Jolla, Calif./Medimmune, Gaithersburg, Md.). Examples of anti-angiogenic agents that act by directly antagonizing or inhibiting angiogenesis inducers and which may be administered in combination with the antibody and antibody compositions of the invention include, but are not limited to, Angiozyme (Ribozyme, Boulder, Colo.), Anti-VEGF antibody (Genentech, S. San Francisco, Calif.), PTK-787/ZK-225846 (Novartis, Basel, Switzerland), SU-101 (Sugen, S. San Francisco, Calif.), SU-5416 (Sugen/Pharmacia Upjohn, Bridgewater, N.J.), and SU-6668 (Sugen). Other anti-angiogenic agents act to indirectly inhibit angiogenesis. Examples of indirect inhibitors of angiogenesis which may be administered in combination with the antibody and antibody compositions of the invention include, but are not limited to, IM-862 (Cytran, Kirkland, Wash.), Interferon-alpha, IL-12 (Roche, Nutley, N.J.), and Pentosan polysulfate (Georgetown University, Washington, D.C.).

In particular embodiments, the use of antibody and antibody compositions of the invention in combination with anti-angiogenic agents is contemplated for the treatment, prevention, and/or amelioration of cancers and other hyperproliferative disorders.

In a further embodiment, the antibody and antibody compositions of the invention are administered in combination with an antiviral agent. Antiviral agents that may be administered with the antibody and antibody compositions of the invention include, but are not limited to, acyclovir, ribavirin, amantadine, and remantidine.

In certain embodiments, Therapeutics of the invention are administered in combination with antiretroviral agents, nucleoside/nucleotide reverse transcriptase inhibitors (NRTIs), non-nucleoside reverse transcriptase inhibitors (NNRTIs), and/or protease inhibitors (PIs). NRTIs that may be administered in combination with the Therapeutics of the invention, include, but are not limited to, RETROVIR™ (zidovudine/AZT), VIDEX™ (didanosine/ddI), HIVWD™ (zalcitabine/ddC), ZERIT™ (stavudine/d4T), EPIVIR™ (lamivudine/3TC), and COMBIVIR™ (zidovudine/lamivudine). NNRTIs that may be administered in combination with the Therapeutics of the invention, include, but are not limited to, VIRAMUNE™ (nevirapine), RESCRIPTOR™ (delavirdine), and SUSTIVA™ (efavirenz). Protease inhibitors that may be administered in combination with the Therapeutics of the invention, include, but are not limited to, CRIXIVAN™ (indinavir), NORVIR™ (ritonavir), INVIRASE™ (saquinavir), and VIRACEPT™ (nelfinavir). In a specific embodiment, antiretroviral agents, nucleoside reverse transcriptase inhibitors, non-nucleoside reverse transcriptase inhibitors, and/or protease inhibitors may be used in any combination with Therapeutics of the invention to treat AIDS and/or to prevent or treat HIV infection.

In a further embodiment, the antibody and antibody compositions of the invention are administered in combination with an antibiotic agent. Antibiotic agents that may be administered with the antibody and antibody compositions of the invention include, but are not limited to, amoxicillin, aminoglycosides, beta-lactam (glycopeptide), beta-lactamases, Clindamycin, chloramphenicol, cephalosporins, ciprofloxacin, ciprofloxacin, erythromycin, fluoroquinolones, macrolides, metronidazole, penicillins, quinolones, rifampin, streptomycin, sulfonamide, tetracyclines, trimethoprim, trimethoprim-sulfamthoxazole, and vancomycin.

In other embodiments, antibody and antibody compositions of the invention may be administered in combination with anti-opportunistic infection agents. Anti-opportunistic agents that may be administered in combination with the antibody and antibody compositions of the invention, include, but are not limited to, TRIMETHOPRIM-SULFAMETHOXAZOLE™, DAPSONE™, PENTAMIDINE™, ATOVAQUONE™, ISONIAZID™, RIFAMPIN™, PYRAZINAMIDE™, ETHAMBUTOL™, RIFABUTIN™, CLARITHROMYCIN™, AZITHROMYCIN™, GANCICLOVIR™, FOSCARNET™, CIDOFOVIRT™, FLUCONAZOLE™, ITRACONAZOLE™, KETOCONAZOLE™, ACYCLOVIR™, FAMCICOLVIR™, PYRIMETHAMINE™, LEUCOVORIN™, NEUPOGEN™ (filgrastim/G-CSF), and LEUKINE™ (sargramostim/GM-CSF). In a specific embodiment, antibody and antibody compositions of the invention are used in any combination with TRIMETHOPRIM-SULFAMETHOXAZOLE™, DAPSONE™, PENTAMIDINE™, and/or ATOVAQUONE™ to prophylactically treat, prevent, and/or diagnose an opportunistic Pneumocystis carinii pneumonia infection. In another specific embodiment, antibody and antibody compositions of the invention are used in any combination with ISONIAZID™, RIFAMPIN™, PYRAZINAMIDE™, and/or ETHAMBUTOL™ to prophylactically treat, prevent, and/or diagnose an opportunistic Mycobactenum avium complex infection. In another specific embodiment, antibody and antibody compositions of the invention are used in any combination with RIFABUTIN™, CLARITHROMYCIN™, and/or AZITHROMYCIN™ to prophylactically treat, prevent, and/or diagnose an opportunistic Mycobacterium tuberculosis infection. In another specific embodiment, antibody and antibody compositions of the invention are used in any combination with GANCICLOVIR™, FOSCARNE™, and/or CIDOFOVIR™ to prophylactically treat, prevent, and/or diagnose an opportunistic cytomegalovirus infection. In another specific embodiment, antibody and antibody compositions of the invention are used in any combination with FLUCONAZOLE™, ITRACONAZOLE™, and/or KETOCONAZOLE™ to prophylactically treat, prevent, and/or diagnose an opportunistic fungal infection. In another specific embodiment, antibody and antibody compositions of the invention are used in any combination with ACYCLOVIR T and/or FAMCICOLVIR™ to prophylactically treat, prevent, and/or diagnose an opportunistic herpes simplex virus type I and/or type 11 infection. In another specific embodiment, antibody and antibody compositions of the invention are used in any combination with PYRIMETHAMINE™ and/or LEUCOVORIN™ to prophylactically treat, prevent, and/or diagnose an opportunistic Toxoplasma gondii infection. In another specific embodiment, antibody and antibody compositions of the invention are used in any combination with LEUCOVORIN™ and/or NEUPOGEN™ to prophylactically treat, prevent, and/or diagnose an opportunistic bacterial infection.

In an additional embodiment, the antibody and antibody compositions of the invention are administered alone or in combination with an anti-inflammatory agent. Anti-inflammatory agents that may be administered with the antibody and antibody compositions of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

The antibodies and antibody compositions of the invention may be administered alone or in combination with other adjuvants. Adjuvants that may be administered with the antibody and antibody compositions of the invention include, but are not limited to, alum, alum plus deoxycholate (ImmunoAg), MTP-PE (Biocine Corp.), QS21 (Genentech, Inc.), BCG, and MPL. In a specific embodiment, antibody and antibody compositions of the invention are administered in combination with alum. In another specific embodiment, antibody and antibody compositions of the invention are administered in combination with QS-21. Further adjuvants that may be administered with the antibody and antibody compositions of the invention include, but are not limited to, Monophosphoryl lipid immunomodulator, AdjuVax 100a, QS-21, QS-18, CRL1005, Aluminum salts, MF-59, and Virosomal adjuvant technology. Vaccines that may be administered with the antibody and antibody compositions of the invention include, but are not limited to, vaccines directed toward protection against MMR (measles, mumps, rubella), polio, varicella, tetanus/diptheria, hepatitis A, hepatitis B, haemophilus influenzae B, whooping cough, pneumonia, influenza, Lyme's Disease, rotavirus, cholera, yellow fever, Japanese encephalitis, poliomyelitis, rabies, typhoid fever, and pertussis, and/or PNEUMOVAX-23™. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In another specific embodiment, antibody and antibody compositions of the invention are used in combination with PNEUMOVAX-23™ to treat, prevent, and/or diagnose infection and/or any disease, disorder, and/or condition associated therewith. In one embodiment, antibody and antibody compositions of the invention are used in combination with PNEUMOVAX-23™ to treat, prevent, and/or diagnose any Gram positive bacterial infection and/or any disease, disorder, and/or condition associated therewith. In another embodiment, antibody and antibody compositions of the invention are used in combination with PNEUMOVAX-23™ to treat, prevent, and/or diagnose infection and/or any disease, disorder, and/or condition associated with one or more members of the genus Enterococcus and/or the genus Streptococcus. In another embodiment, antibody and antibody compositions of the invention are used in any combination with PNEUMOVAX-23™ to treat, prevent, and/or diagnose infection and/or any disease, disorder, and/or condition associated with one or more members of the Group B streptococci. In another embodiment, antibody and antibody compositions of the invention are used in combination with PNEUMOVAX-23™ to treat, prevent, and/or diagnose infection and/or any disease, disorder, and/or condition associated with Streptococcus pneumoniae.

In a preferred embodiment, the antibody and antibody compositions of the invention are administered in combination with CD40 ligand (CD40L), a soluble form of CD40L (e.g., AVREND™), bioloigically active fragments, variants, or derivatives of CD40L, anti-CD40L antibodies (e.g., agonistic or antagonistic antibodies), and/or anti-CD40 antibodies (e.g., agonistic or antagonistic antibodies).

In another embodiment, antibody and antibody compositions of the invention are administered in combination with an anticoagulant. Anticoagulants that may be administered with the antibody and antibody compositions of the invention include, but are not limited to, heparin, warfarin, and aspirin. In a specific embodiment, antibody and antibody compositions of the invention are administered in combination with heparin and/or warfarin. In another specific embodiment, antibody and antibody compositions of the invention are administered in combination with warfarin. In another specific embodiment, antibody and antibody compositions of the invention are administered in combination with warfarin and aspirin. In another specific embodiment, antibody and antibody compositions of the invention are administered in combination with heparin. In another specific embodiment, antibody and antibody compositions of the invention are administered in combination with heparin and aspirin.

In another embodiment, antibody and antibody compositions of the invention are administered in combination with an agent that suppresses the production of anticardiolipin antibodies. In specific embodiments, the polynucleotides of the invention are administered in combination with an agent that blocks and/or reduces the ability of anticardiolipin antibodies to bind phospholipid-binding plasma protein beta 2-glycoprotein 1 (b2GPI).

In a preferred embodiment, the antibody and antibody compositions of the invention are administered in combination with an antimalarial. Antimalarials that may be administered with the antibody and antibody compositions of the invention include, but are not limited to, hydroxychloroquine, chloroquine, and/or quinacrine.

In a preferred embodiment, the antibody and antibody compositions of the invention are administered in combination with an NSAID. In a preferred embodiment, the antibody and antibody compositions of the invention are administered in combination with indomethacin. In a preferred embodiment, the antibody and antibody compositions of the invention are administered in combination with sodium salicylate.

In a nonexclusive embodiment, the antibody and antibody compositions of the invention are administered in combination with one, two, three, four, five, ten, or more of the following drugs: NRD-101 (Hoechst Marion Roussel), diclofenac (Dimethaid), oxaprozin potassium (Monsanto), mecasermin (Chiron), T-714 (Toyama), pemetrexed disodium (Eli Lilly), atreleuton (Abbott), valdecoxib (Monsanto), eltenac (Byk Gulden), campath, AGM-1470 (Takeda), CDP-571 (Celltech Chiroscience), CM-101 (CarboMed), ML-3000 (Merckle), CB-2431 (KS Biomedix), CBF-BS2 (KS Biomedix), IL-1Ra gene therapy (Valentis), JTE-522 (Japan Tobacco), paclitaxel (Angiotech), DW-166HC (Dong Wha), darbufelone mesylate (Warner-Lambert), soluble TNF receptor 1 (synergen; Amgen), IPR-6001 (Institute for Pharmaceutical Research), trocade (Hoffman-La Roche), EF-5 (Scotia Pharmaceuticals), BIIL-284 (Boehringer Ingelheim), BIIF-1149 (Boehringer Ingelheim), LeukoVax (Inflammatics), MK-671 (Merck), ST-1482 (Sigma-Tau), and butixocort propionate (WarnerLambert).

In a preferred embodiment, the antibody and antibody compositions of the invention are administered in combination with one, two, three, four, five or more of the following drugs: methotrexate, sulfasalazine, sodium aurothiomalate, auranofin, cyclosporine, penicillamine, azathioprine, an antimalarial drug (e.g., as described herein), cyclophosphamide, chlorambucil, gold, ENBREL™ (Etanercept), anti-TNF antibody, LJP 394 (La Jolla Pharmaceutical Company, San Diego, Calif.) and prednisolone.

In an additional embodiment, antibody and antibody compositions of the invention are administered alone or in combination with one or more intravenous immune globulin preparations. Intravenous immune globulin preparations that may be administered with the antibody and antibody compositions of the invention include, but not limited to, GAMMAR™, IVEEGAM™, SANDOGLOBULIN™, GAMMAGARD S/D™, and GAMIMUNE™. In a specific embodiment, antibody and antibody compositions of the invention are administered in combination with intravenous immune globulin preparations in transplantation therapy (e.g., bone marrow transplant).

CD40 ligand (CD40L), a soluble form of CD40L (e.g., AVREND™), biologically active fragments, variants, or derivatives of CD40L, anti-CD40L antibodies (e.g., agonistic or antagonistic antibodies), and/or anti-CD40 antibodies (e.g., agonistic or antagonistic antibodies).

In an additional embodiment, the antibody and antibody compositions of the invention are administered in combination with cytokines. Cytokines that may be administered with the antibody and antibody compositions of the invention include, but are not limited to, GM-CSF, G-CSF, IL2, IL3, IL4, IL5, IL6, IL7, IL10, IL12, IL13, IL15, anti-CD40, CD40L, IFN-alpha, IFN-beta, IFN-gamma, TNF-alpha, and TNF-beta. In preferred embodiments, antibody and antibody compositions of the invention are administered with TRAIL receptor. In another embodiment, antibody and antibody compositions of the invention may be administered with any interleukin, including, but not limited to, IL-1alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, and IL-22. In preferred embodiments, the antibody and antibody compositions of the invention are administered in combination with IL4 and IL10. In other preferred embodiments, the antibody and antibody compositions of the invention are administered in combination with IL2. In preferred embodiments, the antibody and antibody compositions of the invention are administered in combination with G-CSF.

In one embodiment, the antibody and antibody compositions of the invention are administered in combination with one or more chemokines. In specific embodiments, the antibody and antibody compositions of the invention are administered in combination with an α(CxC) chemokine selected from the group consisting of gamma-interferon inducible protein-10 (γIP-10), interleukin-8 (IL-8), platelet factor-4 (PF4), neutrophil activating protein (NAP-2), GRO-α, GRO-β, GRO-γ, neutrophil-activating peptide (ENA-78), granulocyte chemoattractant protein-2 (GCP-2), and stromal cell-derived factor-1 (SDF-1, or pre-B cell stimulatory factor (PBSF)); and/or a β(CC) chemokine selected from the group consisting of: RANTES (regulated on activation, normal T expressed and secreted), macrophage inflammatory protein-1 alpha (MIP-1α), macrophage inflammatory protein-1 beta (MIP-1β), monocyte chemotactic protein-1 (MCP-1), monocyte chemotactic protein-2 (MCP-2), monocyte chemotactic protein-3 (MCP-3), monocyte chemotactic protein-4 (MCP-4) macrophage inflammatory protein-1 gamma (MIP-1β), macrophage inflammatory protein-3 alpha (MIP-3a), macrophage inflammatory protein-3 beta (MIP-3β), macrophage inflammatory protein-4 (IP-4/DC-CK-1/PARC), eotaxin, Exodus, and 1-309; and/or the γ(C) chemokine, lymphotactin. In preferred embodiments, the antibody and antibody compositions of the invention are administered in combination with an agent that increases IFN-gamma and/or caspase activity particularly caspase-8 activity.

In another embodiment, the antibody and antibody compositions of the invention are administered with chemokine beta-8, chemokine beta-1, and/or macrophage inflammatory protein-4. In a preferred embodiment, the antibody and antibody compositions of the invention are administered with chemokine beta-8.

In an additional embodiment, the antibody and antibody compositions of the invention are administered in combination with an IL-4 antagonist. IL-4 antagonists that may be administered with the antibody and antibody compositions of the invention include, but are not limited to: soluble IL-4 receptor polypeptides, multimeric forms of soluble IL-4 receptor polypeptides; anti-IL-4 receptor antibodies that bind the IL-4 receptor without transducing the biological signal elicited by IL-4, anti-IL-4 antibodies that block binding of IL-4 to one or more IL-4 receptors, and muteins of IL-4 that bind IL-4 receptors but do not transduce the biological signal elicited by IL-4. Preferably, the antibodies employed according to this method are monoclonal antibodies (including antibody fragments, such as, for example, those described herein).

In an additional embodiment, the antibody and antibody compositions of the invention are administered in combination with fibroblast growth factors. Fibroblast growth factors that may be administered with the antibody and antibody compositions of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

Agonistic antibodies of the invention can be administered in combination with one or more of the above-described therapeutic agents in the treatment, prevention, amelioration and/or cure of cancers and premalignant conditions.

Demonstration of Therapeutic or Prophylactic Utility of a Composition

The compounds of the invention are preferably tested in vitro, and then in vivo for the desired therapeutic or prophylactic activity, prior to use in humans. For example, in vitro assays which can be used to determine whether administration of a specific antibody or composition of the present invention is indicated, include in vitro cell culture assays in which a patient tissue sample is grown in culture, and exposed to or otherwise administered an antibody or composition of the present invention, and the effect of such an antibody or composition of the present invention upon the tissue sample is observed. In various specific embodiments, in vitro assays can be carried out with representative cells of cell types involved in a patient's disorder, to determine if an antibody or composition of the present invention has a desired effect upon such cell types. Preferably, the antibodies or compositions of the invention are also tested in in vitro assays and animal model systems prior to administration to humans.

Antibodies or compositions of the present invention for use in therapy can be tested for their toxicity in suitable animal model systems, including but not limited to rats, mice, chicken, cows, monkeys, and rabbits. For in vivo testing of an antibody or composition's toxicity any animal model system known in the art may be used.

Antibodies or compositions of the invention can be tested for their ability to reduce tumor formation in in vitro, ex vivo and in vivo assays. Antibodies or compositions of the invention can also be tested for their ability to inhibit viral replication or reduce viral load in in vitro and in vivo assays. Antibodies or compositions of the invention can also be tested for their ability to reduce bacterial numbers in in vitro and in vivo assays known to those of skill in the art. Antibodies or compositions of the invention can also be tested for their ability to alleviate of one or more symptoms associated with cancer, an immune disorder (e.g., an inflammatory disease), a neurological disorder or an infectious disease. Antibodies or compositions of the invention can also be tested for their ability to decrease the time course of the infectious disease. Further, antibodies or compositions of the invention can be tested for their ability to increase the survival period of animals suffering from disease or disorder, including cancer, an immune disorder or an infectious disease. Techniques known to those of skill in the art can be used to analyze the function of the antibodies or compositions of the invention in vivo.

Efficacy in treating or preventing viral infection may be demonstrated by detecting the ability of an antibody or composition of the invention to inhibit the replication of the virus, to inhibit transmission or prevent the virus from establishing itself in its host, or to prevent, ameliorate or alleviate the symptoms of disease a progression. The treatment is considered therapeutic if there is, for example, a reduction in viral load, amelioration of one or more symptoms, or a decrease in mortality and/or morbidity following administration of an antibody or composition of the invention.

Antibodies or compositions of the invention can be tested for their ability to modulate the biological activity of immune cells by contacting immune cells, preferably human immune cells (e.g., T-cells, B-cells, and Natural Killer cells), with an antibody or composition of the invention or a control compound and determining the ability of the antibody or composition of the invention to modulate (i.e., increase or decrease) the biological activity of immune cells. The ability of an antibody or composition of the invention to modulate the biological activity of immune cells can be assessed by detecting the expression of antigens, detecting the proliferation of immune cells (i.e., B-cell proliferation), detecting the activation of signaling molecules, detecting the effector function of immune cells, or detecting the differentiation of immune cells. Techniques known to those of skill in the art can be used for measuring these activities. For example, cellular proliferation can be assayed by 3H-thymidine incorporation assays and trypan blue cell counts. Antigen expression can be assayed, for example, by immunoassays including, but not limited to, competitive and non-competitive assay systems using techniques such as western blots, immunohistochemistry radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays and FACS analysis. The activation of signaling molecules can be assayed, for example, by kinase assays and electrophoretic shift assays (EMSAs). In a preferred embodiment, the ability of an antibody or composition of the invention to induce B-cell proliferation is measured. In another preferred embodiment, the ability of an antibody or composition of the invention to modulate immunoglobulin expression is measured.

Panels/Mixtures

The present invention also provides for mixtures of antibodies (including scFvs and other molecules comprising, or alternatively consisting of, antibody fragments or variants thereof) that immunospecifically bind to TRAIL receptor or a fragment or variant thereof, wherein the mixture has at least one, two, three, four, five or more different antibodies of the invention. In specific embodiments, the invention provides mixtures of at least 2, preferably at least 4, at least 6, at least 8, at least 10, at least 12, at least 15, at least 20, or at least 25 different antibodies that immunospecifically bind to TRAIL receptor or fragments or variants thereof, wherein at least 1, at least 2, at least 4, at least 6, or at least 10, antibodies of the mixture is an antibody of the invention. In a specific embodiment, each antibody of the mixture is an antibody of the invention.

The present invention also provides for panels of antibodies (including scFvs and other molecules comprising, or alternatively consisting of, antibody fragments or variants thereof) that immunospecifically bind to TRAIL receptor or a fragment or variant thereof, wherein the panel has at least one, two, three, four, five or more different antibodies of the invention. In specific embodiments, the invention provides for panels of antibodies that have different affinities for TRAIL receptor, different specificities for TRAIL receptor, or different dissociation rates. The invention provides panels of at least 10, preferably at least 25, at least 50, at least 75, at least 100, at least 125, at least 150, at least 175, at least 200, at least 250, at least 300, at least 350, at least 400, at least 450, at least 500, at least 550, at least 600, at least 650, at least 700, at least 750, at least 800, at least 850, at least 900, at least 950, or at least 1000, antibodies. Panels of antibodies can be used, for example, in 96 well plates for assays such as ELISAs.

The present invention further provides for compositions comprising, one or more antibodies (including molecules comprising, or alternatively consisting of antibody fragments or variants of the invention). In one embodiment, a composition of the present invention comprises, one, two, three, four, five, or more antibodies that comprise or alternatively consist of, a polypeptide having an amino acid sequence of any one or more of the VH domains of a heavy chain expressed by one or more of the cell lines referred to in Table 1, or a variant thereof. In another embodiment, a composition of the present invention comprises, one, two, three, four, five, or more antibodies that comprise, or alternatively consist of, a polypeptide having an amino acid sequence of any one or more of the VH CDR is of a heavy chain expressed by one or more of the cell lines referred to in Table 1, or a variant thereof. In another embodiment, a composition of the present invention comprises, one, two, three, four, five or more antibodies that comprise, or alternatively consist of, a polypeptide having an amino acid sequence of any one or more of the VH CDR2s of a heavy chain expressed by one or more of the cell lines referred to in Table 1, or a variant thereof. In a preferred embodiment, a composition of the present invention comprises, one, two, three, four, five, or more antibodies that comprise, or alternatively consist of, a polypeptide having an amino acid sequence of any one or more of the VH CDR3s as of a heavy chain expressed by one or more of the cell lines referred to in Table 1, or a variant thereof.

Other embodiments of the present invention providing for compositions comprising, one or more antibodies (including molecules comprising, or alternatively consisting of antibody fragments or variants of the invention) are listed below. In another embodiment, a composition of the present invention comprises, one, two, three, four, five, or more antibodies that comprise, or alternative consist of, a polypeptide having an amino acid sequence of any one or more of the VL domains of a light chain expressed by one or more of the cell lines referred to in Table 1, or a variant thereof. In another embodiment, a composition of the present invention comprises, one, two, three, four, five, or more antibodies that comprise, or alternatively consist of, a polypeptide having an amino acid sequence of any one or more of the VL CDR1s of a light chain expressed by one or more of the cell lines referred to in Table 1, or a variant thereof. In another embodiment, a composition of the present invention comprises, one, two, three, four, five, or more antibodies that comprise, or alternatively consist of, a polypeptide having an amino acid sequence of any one or more of the VL CDR2s of a light chain expressed by one or more of the cell lines referred to in Table 1, or a variant thereof. In a preferred embodiment, a composition of the present invention comprises, one, two, three, four, five, or more antibodies that comprise, or alternatively consist of, a polypeptide having an amino acid sequence of any one or more of the VL CDR3s of a light chain expressed by one or more of the cell lines referred to in Table 1, or a variant thereof.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that can be used in the above methods. In one embodiment, a kit comprises an antibody of the invention, preferably a purified antibody, in one or more containers. In an alterative embodiment, a kit comprises an antibody fragment that immunospecifically binds to TRAIL receptor polypeptides or fragments or variants thereof. In a specific embodiment, the kits of the present invention contain a substantially isolated TRAIL receptor polypeptide or fragment or variant thereof as a control. Preferably, the kits of the present invention further comprise a control antibody which does not react with any, some or all TRAIL receptors. In another specific embodiment, the kits of the present invention contain a means for detecting the binding of an antibody to TRAIL receptor polypeptides (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate). In specific embodiments, the kit may include a recombinantly produced or chemically synthesized TRAIL receptor. The TRAIL receptor provided in the kit may also be attached to a solid support. In a more specific embodiment the detecting means of the above-described kit includes a solid support to which TRAIL receptor is attached. Such a kit may also include a non-attached reporter-labeled anti-human antibody. In this embodiment, binding of the antibody to TRAIL receptor can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with a TRAIL receptor, and means for detecting the binding of TRAIL receptor polypeptides to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having surface-bound TRAIL receptors obtained by the methods of the present invention. After TRAIL receptor polypeptides bind to a specific antibody, the unbound serum components are removed by washing, reporter-labeled anti-human antibody is added, unbound anti-human antibody is removed by washing, and a reagent is reacted with reporter-labeled anti-human antibody to bind reporter to the reagent in proportion to the amount of bound anti-TRAIL receptor antibody on the solid support. Typically, the reporter is an enzyme which is detected by incubating the solid phase in the presence of a suitable fluorometric, luminescent or colorimetric substrate.

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant TRAIL receptor, and a reporter-labeled anti-human antibody for detecting surface-bound anti-TRAIL receptor antibody.

Placental Expression of TRAIL Receptors

The expression of tumor necrosis family receptors and ligands in whole placenta and in placental macrophage and trophoblast cell lines have been carefully examined. It has been shown that trophoblasts express TR7 and TR5 but not TR10 are entirely resistant to killing by recombinant TRAIL whereas macrophages, which express TR4, TR7 and TR10 but not TR5, are sensitive (Phillips et al., J. Immunol 15:6053-9 (1999) which is incorporated in its entirety by refrence herein). Thus the methods for using anti-TRAIL receptor antibodies described herein, may also be used on placenta and placental cell types (e.g., macrophagges and trophoblast cells) to prevent, treat, diagnose, ameliorate, or monitor diseases and disorders of the placenta placental cell types.

Gene Therapy

In a specific embodiment, nucleic acids comprising sequences encoding antibodies or functional derivatives thereof, are administered to treat, inhibit or prevent a disease or disorder associated with aberrant expression and/or activity of TRAIL Receptors and/or its ligands (e.g., TRAIL), by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 1 l(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises, or alternatively consists of, nucleic acids encoding an antibody, said nucleic acids being part of an expression vector that expresses the antibody or fragments or chimeric proteins or heavy or light chains thereof in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is an scFv; alternatively, the nucleic acid sequences include sequences encoding both the heavy and light chains, or fragments or variants thereof, of an antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the ark e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22715; WO92/203 16; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijistra et al., Nature 342:435-438 (1989)).

In a specific embodiment, viral vectors that contains nucleic acid sequences encoding an antibody of the invention or fragments or variants thereof are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:29 1-302 (1994), which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., J. Clin. Invest. 93:644-651(1994); Klein et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, Meth. Enzymol. 217:599-718 (1993); Cohen et al., Meth. Enzymol. 217:718-644 (1993); Clin. Pharma. Ther. 29:69-92m (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody or fragment thereof are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 7 1:973-985 (1992); Rheinwald, Meth. Cell Biol. 21A:229 (1980); and Pittelkow and Scott, Mayo Clinic Proc. 71:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### EXAMPLES

### Example 1

### Hybridoma Cell Lines

Table 1 lists the names of the hybridomas generated from Xenomouse™ strains that had been immunized with TRAIL receptor polypetides or fragments or variants thereof including TRAIL receptor fusion polypeptides which express anti-TRAIL receptor antibodies. Several hybridoma fusion panels were generated from mice that had been immunized with TR4-Fc and/or TR10 Fe fusion proteins. The Fe fusion proteins are described in more detail in Example 2. Fusions 1, 2, and 3 were obtained using lymphocytes from mice that had received seven injections of 10 micrograms of TR4-Fc fusion protein in Ribi adjuvant at 3 to 5 day intervals. The injections were administered in the footpad. Mice were sacrificed 4 days after the last injection. Fusions 4 and 5 were obtained using lymphocytes from mice that had received 5 injections of 10 micrograms of TR4-Fc fusion protein in Freund's adjuvant followed by three injections of 10 micrograms of TR-10 Fe fusion protein in Freund's adjuvant at 3 to 5 day intervals. The injections were administered in the footpad. Mice were sacrificed 4 days after the last injection. Fusions 6 and 7 were obtained using lymphocytes from mice that had received 5 injections of 10 micrograms of TR4-Fc fusion protein in Freund's adjuvant followed by a single injection of 10 micrograms of TR-10 Fe fusion protein in Freund's adjuvant at approximately 2 week intervals. The injections were administered in the intraperitoneally and in the base of the tail. Mice were sacrificed 4 days after the last injection. Fusions 8 and 9 were obtained using lymphocytes from mice that had received 5 injections of 10 micrograms of TR4-Fc fusion protein in Freund's adjuvant followed by two injections of 10 micrograms of TR-10 Fe fusion protein in Freund's adjuvant at approximately 2 week intervals. The injections were administered in intraperitoneally and in the base of the tail. Mice were sacrificed 4 days after the last injection.

Clone designations that contain only one period are primary hybridomas isolates (e.g., 3.1) whereas clone designations with two periods (e.g., 3.1.6) indicate hybridomas subcloned from a primary hybridoma isolate whose clone designation is equal to the first two numbers of the subclone designation (e.g., 3.1). When appropriate, ATCC Deposit Numbers corresponding to particular cell lines are also listed.

Fusion XF2

Hybridomas in Fusion XF2 were obtained using lymphocytes from Xenomice that had received 5 immunizations with 10 micrograms of TR4-Fc fusion protein. The TR4 protein was first delivered to the animal in complete Freunds' adjuvant via two 100 microliter injections, one at the base of the tail (BOT) and one intrapaeritoneal (IP) injection. This was followed by three subsequent immunizations also 10 micrograms, in 200 microliters delivered via BOT and IP injection of TR4-Fc protein in Incomplete Freund's adjuvant (IFA), each separated by two weeks. Two weeks after the last injection in IFA, mice were each given one injection of TR4-Fc in PBS, also 10 micrograms, in 200 microliters delivered via BOT and IP injection. Three days after the last immunization, the animal were sacrificed and spleens and lymph nodes were harvested for fusion. A total of 2,784 hybridoam were obtained of which 145 screened positive for binding to TR4-Fc. Of these 145, 54 were determined to bind the TR4 portion, and not the Fe portion, of TR4-Fc. 13 of these 54 clones were able to induce apoptosis of ST486 cells (see Example 4 below). These thirteen clones are designated XF2-1A12, XF2-1G5, XF2-2D2, XF2-2H1, XF24C2, XF24F7, XF24G8, XF24H5, XF2-4H11, XF2-18A10, XF2-19C10 and XF2-23H7. Of these, all but XF2-1G3, XF24C2, XF2-2H1 and XF2-1A12 are also able to bind TR4 from cynomolgous monkey in addition to human TR4 and all but XF2-2H1 and XF2-1A12 are able to bind TR4 from chimpanzee.

### Example 2

Biacore Analysis of the Affinity of TRAIL Receptor Binding Polypeptides

Materials

BIAcore 2000 instrument
BIAcore 2000 control software, version 3.1.1
BIAevaluation, version 3.1
BIAcore CM5 Sensor Chip, Cat # BR-1000-14 Lot# 0364 (BIAcore)
HBS-EP Buffer
Amine Coupling Kit Cat# BR-1000-50 (BIAcore)
   EDC, #1048-950345(BIAcore)
   NHS, #1048-950345(BIAcore)
   Ethanolamine, # 1048-950345(BIAcore)
10 mM Acetate, pH 4.0 Cat# BR1003-50 Lot#1821-9503844(BIAcore)
TRAIL-FLAG (Alexis Biochemicals Cat# 522-003-C010 #L4793/a)
The temperature was 25° C. for all experiments.
   Methods

TR4, TR5, TR7 and TR10 (in the form of Fe fusion proteins) were immobilized on individual flow cells of a BIAcore sensor chip. The TR4-Fc fusion protein comprised residues M1-I240 of TR4 (SEQ ID NO:1). Post translational processing of this fusion protein results in a TR4-Fc fusion protein that comprises residues A109-I240 of TR4 (SEQ ID NO:1). The TR5-Fc fusion protein comprised residues R70-S282 of TR5 (SEQ ID NO:2). This protein was expressed in a baculovirus expression system that utilized the GP signal peptide. Thus, post-translational processing of this fusion protein results in a TR5-Fc fusion protein that comprises the last 3 residues of the GP signal peptide (Ala-Asp-Pro) fused to R-70-S282 of TR5 (SEQ ID NO:2) fused to the Fe region. The TR7-Fc fusion protein comprised residues E-52-G184 of TR7 (SEQ ID NO:3). This protein was expressed in a baculovirus expression system that utilized the GP signal peptide. Thus, post-translational processing of this fusion protein results in a TR7-Fc fusion protein that comprises the last 3 residues of the GP signal peptide (Ala-Asp-Pro) fused to E-52-G184 of TR5 (SEQ ID NO:3) fused to the Fe region. The TR10-Fc fusion protein comprised residues M1-G204 of TR10 (SEQ ID NO:4). Post translational processing of this fusion protein results in a TR10-Fc fusion protein that comprises residues A56-G204 of TR10 (SEQ ID NO:4).

Amine coupling was used to covalently bind each receptor (Fc) to the dextran matrix on the CM5 sensor chip. The optimal pH for this coupling was analyzed using preconcentration experiments ranging from pH 4-7 and was determined to be 4.0 based on the slope of the binding.

The actual coupling was performed using the manual injection mode. A target level of ˜2000 RU was set as the goal for all flow cells. (This did vary from 2000-3100 depending on the molecular weight of the receptor). The concentration of all receptors for immobilization was 10 ug/ml in 10 mM acetate, pH 4.0. The entire immobilization experiment was performed at 4 microliters/min. Contact time for the EDC/NHS injection was 7 minutes. The ethanolamine was injected for 7 minutes.

The screening was performed with the following procedures. The flow rate for the entire binding cycle was 25 microliters/minute. All hybridoma supernatants were diluted 1:10 in HBS-EP and flown through all four cells with immobilized TRAIL receptors. Each sample was in contact with the receptors for 4 minutes. Regeneration was performed using 15 microliters of 25 mM NaOH. Successful regeneration was considered as not only removing the antibody, but also not denaturing the immobilized receptor.

The positive control for this screening experiment was an identical (in flow rate and length of time) injection of the soluble TRAIL ligand. The concentration was 1 microgram/mL. The negative control was a 1:10 dilution of the DMEM media of which the supernatants were composed. Data was analyzed using the BIAevaluation software package.

Several of the monoclonal antibodies expressed by the hybridoma cell lines shown in Table 1 were purified and tested by BIAcore analysis for their ability to bind Fc-fusion proteins of TR4, TR5, TR7 and TR10. In adition to testing the crude hybridoma supernatants, several of the antibodies were purifed from serum free cell culture supernatants by passage over, and elution from a Protein A column, prior to BIAcore analysis. As an example, BIAcore analysis showed that the 7.3 antibody preferentially bound TR-4Fc compared to TR5-Fc, TR7-Fc, and TR10-Fc.

### Example 3

Inhibition of Binding of Biotinylated-TRAIL to TR4

I. Materials:

10×PBS (Quality Biological Cat 130-069-161, Lot 708712)
Immulon 4 microplate (Dynex Cat 3855, Lot ND540319)
Bovine Serum Albumin fraction V (Sigma, #58H0456)
Tri Hydroxy Methyl Amino Methane (TRIS BASE)
Tween 20 (Sigma)
Goat anti-human Fe (Sigma, 1-2136, #89H4871)
TR-4:Fc (as described above)
Biotinylated TRAIL (AM100200-Peprotech)
WRP-Streptavidin (Vector, #L0328)
TMB Peroxidase Microwell Substrate System (KPL, Kirkegaard & Perry Laboratories, Inc.)
H₂SO₄ (Fisher)
96 well dilution plate (Costar)
   II. Buffers:
Coating buffer (1×PBS)
Blocking buffer (3% BSA in PBS)
All-purpose Diluent (1% BSA in PBST)
Washing buffer (0.1% Tween 20 and 1×PBS)
   III. Methods

Goat anti-human Fc was diluted to 0.1 micrograms/ml in coating buffer. The Immulon 4 microplate was coated with 100 microliters per well of the Goat anti-human Fe solution and incubated overnight at 4° C. The coating solution was decanted from the plate, and blocking solution was dispensed at 200 microliters per well. The plate was incubated at room temperature for 1 hour. After the 1 hour incubation period, the blocking solution was decanted from the plate and 1 microgram/mL of TR4-Fc was dispensed at 100 microliters/well and incubated for 2 hours at room temperature. After the incubation, the plate was washed five times manually using a Wheaton manifold.

Hybridoma supernatants were (previously) prepared in a low binding dilution plate using diluent. The supernatants were prepared in duplicate and were diluted from the stock concentration with 2.5 fold dilutions for the 7 subsequent wells. If a purified form of the antibody was available, the starting concentration was 5 micrograms/mL. The positive control (TR4-Fc) was diluted from 5 micrograms/mL. 100 microliters was transferred into the ELISA plate and pre-incubated for 30 minutes at room temperature. 20 microliters of biotinylated TRAIL was added at 5 micrograms/mL to the 100 μL of the supernatant and mixed. The combined 120 microliters incubated for 2 hours at RT.

After the two-hour incubation the washing cycle was repeated and the plate decanted and blotted. HRP-streptavidin was diluted 1:2000 and 100 microliters per well was dispensed. Incubation was for one hour at room temperature. Meanwhile, equal amounts of the TMB peroxidase substrate and the peroxidase solution B were withdrawn and the solutions were equilibrated to room temperature.

After the one-hour incubation, the plate was decanted and washed with PBST five times and blotted. The TMB peroxidase substrate and the peroxidase solution B were combined and 100 microliters was dispensed to each well. The color developed at room temperature for 15 minutes. The color development was quenched by adding 50 microliters of the 1 M H₂SO₄ to each well. The plate was immediately read at 450 nm using the spectrometer from Molecular Devices.

The IC-50, i.e., the concentration of purified antibody that resulted in 50% inhibition of plateau binding, of a few of the antibodies tested is presented in Table 7. Results obtained from two different assays are presented as assay 1 and assay 2. The starting concentration of each antibody in this assay was 5 micrograms/milliliter. For comparison purposes, a TR4 polypeptide was used as a sample in this assay.

TABLE 7
IC-50 of antibodies in the Inhibition of Binding
of Biotinylated-TRAIL to TR4 Assay

| | Assay 1 | Asssay 2 |
|---|---|---|
| Antibody | IC-50 (micrograms/milliliter) | IC-50 (micrograms/milliliter) |
| 7.1.3 | 0.152 | 0.068 |
| 7.2 | 1.959 | Not Tested |
| 7.3.2 | 0.157 | 0.159 |
| 7.8 | 10.3 | Not Tested |
| 7.10 | 4.3 | Not Tested |
| 7.10.2 | Not Tested | 0.244 |
| 7.12.1 | 0.134 | 0.127 |
| 8.3.1 | Not Tested | 2.7 |
| 8.3.2 | Not Tested | 2.9 |
| TR4 | 0.491 | 0.284 |

### Example 4

Assay for Ability of Anti-TRAIL-R1 Antibodies to Induce Apoptosis

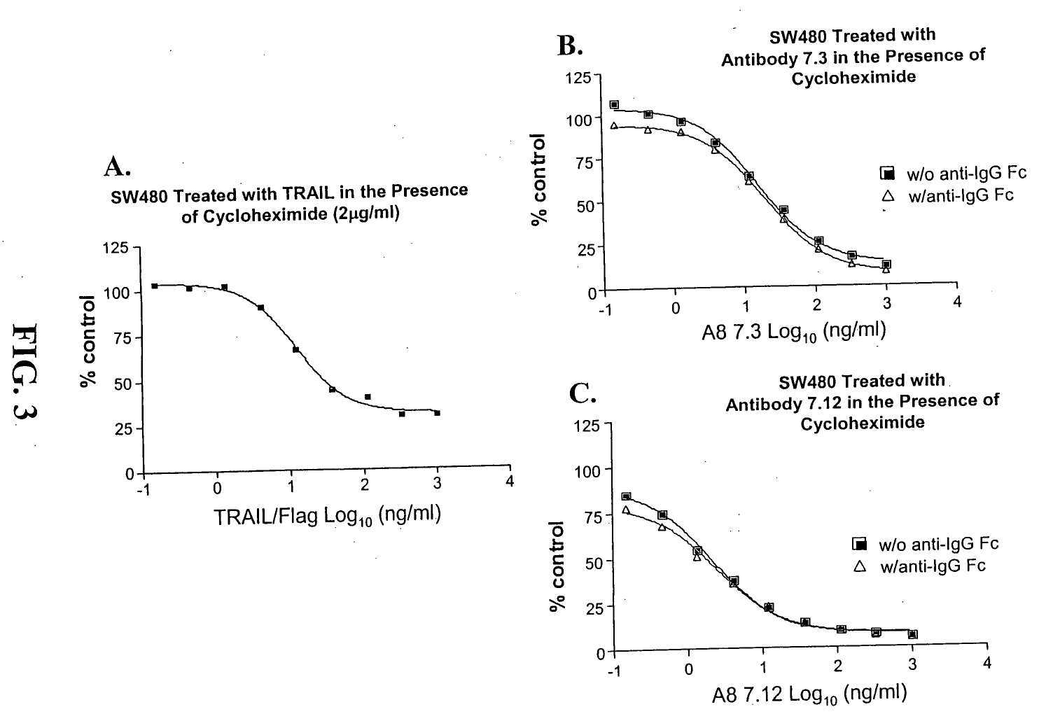

Anti-TRAIL receptor antibodies were tested for their ability to induce apoptosis of TRAIL receptor expressing cells, alone or in combination with chemotherapeutic or cross-linking agents. Briefly, hybridoma supernatants or purified antibodies were tested for activity to induce TRAIL receptor mediated apoptosis of TR4 expressing cell lines, SW480 and HeLa. HT1080 fibrosarcoma cell line, which failed to express TR4, was used as a negative control (See FIG. 3).

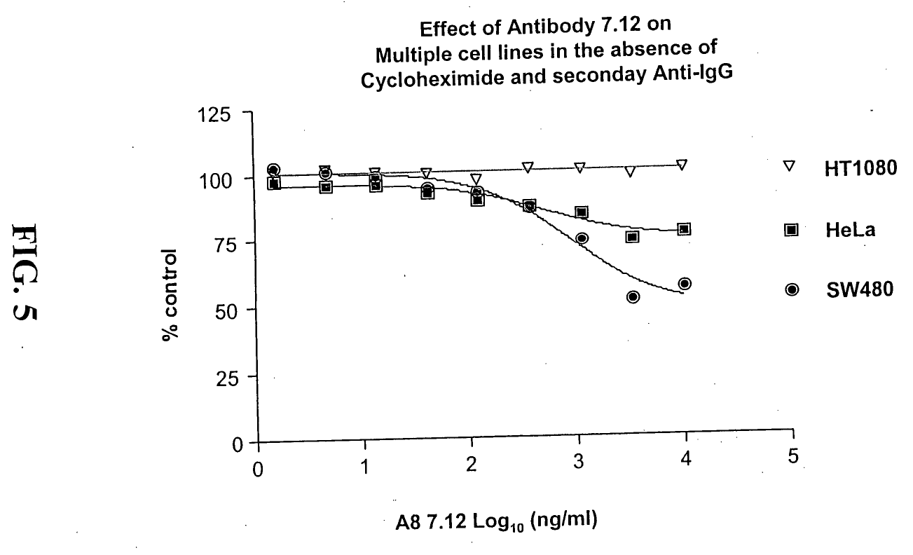

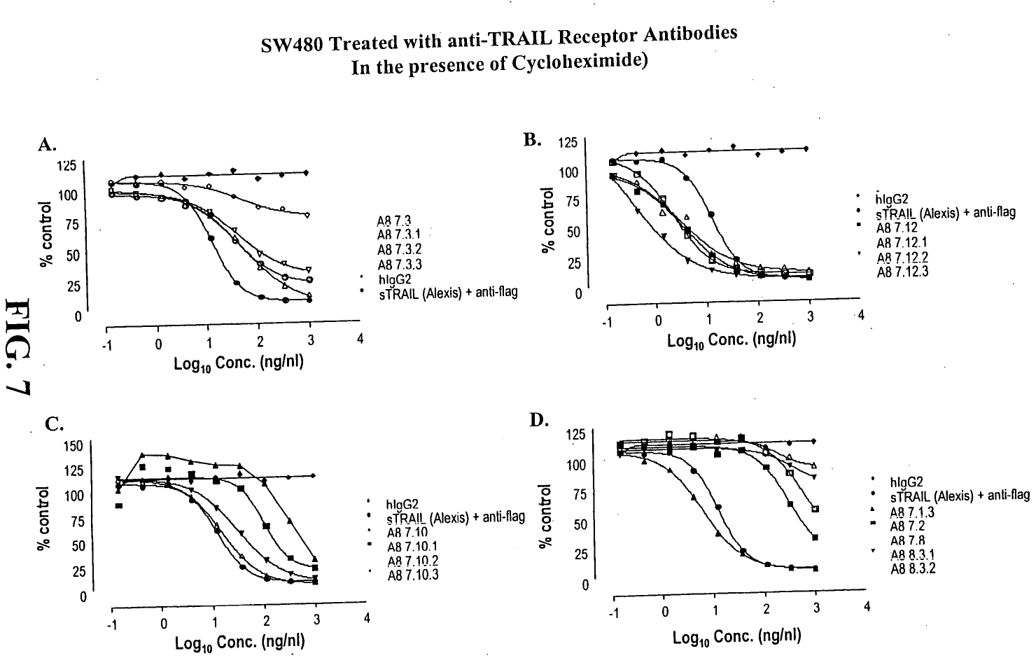

To induce apoptosis, either HeLa or SW480 cells were incubated with the indicated concentration of monoclonal antibodies (e.g., 7.3 or 7.12) or a human IgG2a control antibody. One day prior to assay, cells (0.3×10⁶ cells/ml; 100 ul/well) were seeded into wells of a 96-well plate and allowed to adhere overnight. The following day, the test antibody was added either in the presence or absence of 2.0 micrograms/ml cycloheximide (Sigma R75010-7). In some experiments, the potency of anti-TRAIL receptor monoclonal antibody was compared to rhuTRAIL-FLAG protein (Alexis Biochemicals). rhuTRAIL was used at the indicated concentrations in the presence of anti-FLAG enhancer antibody at 2 micrograms/ml (See FIGS. 2, 3, 4, 5, and 7). The effect of secondary crosslinking was also assessed by measuring the ability of the monoclonal antibodies 7.3 or 7.12 to kill cells alone, or in the presence of a secondary goat-anti-human Ig Fc specific antibody (SIGMA) (See FIGS. 2, 3, 4, and 5). The secondary crosslinking antibody was added to cells at an equivalent concentration as the test monoclonal antibody. The ability of a chemotherapeutic agent to sensitize cells to killing via the monoclonal antibody 7.12, was assessed by treating either Hela or SW480 cells with monoclonal antibody 7.12 in the presence of Topotecan (Hycamtin, Smith Kline Beecham NDC 0007-4201-01) (See FIG. 6). The data in FIGS. 2 and 3 show that monoclonal antibodies 7.3 and 7.12 are reduce cell viability to 50% of the control value at lower concentrations than rhuTRAIL was able to reduce cell viability to 50% of the control. Further, monoclonal antibodies 7.3 and 7.12 were able to kill cells equally well in the presence or absence of secondary crosslinking reagents (i.e., anti IgG Fe reagent)

Assays were performed for 16-18 hrs at 37° C., after which viability was revealed using the reagent, Alamar Blue (Biosource, cat. # DAL11100) using conditions suggested by the manufacturer. Alamar Blue fluorescence was detected using the CytoFluor fluorescence reader at 530 nm excitation and 590 emission. Results are expressed as a percent viability compared to untreated cells (see FIGS. 1-7). Cell viability may also be measured using other methods described herein or otherwise known in the art. For example, cell viability may be measured using the CellTiter-Glo®Luminescent Cell Viability Assay available from Promega according to the manufacturer's instructions.

The data illustrated in FIG. 3 show that treatment of cells with Topotecan and monoclonal antibody 7.12 results in stong synergistic killing of cells expressing TR4. Other chemotherapeutics tested in this assay included 5-Fluorouracil, Etoposide, Taxol, Cisplatin, Cytabarine (Cytosar), and IFN gamma.

When monoclonal antibody 7.12 was tested on HeLa cells, no synergistic killing was observed with 5-Fluorouracil, Etoposide, or Cytarabine. Weak synergy was observed with IFN-gamma and Taxol. When monoclonal antibody 7.12 was tested on SW480 cells, no synergistic killing was observed with 5-Fluorouracil, IFN-gamma or Cytarabine. Weak synergy was observed with Taxol, Etoposide, or Cisplatin (data not shown). Thus, these data show that antibody 7.12 administered with particular chemotherapeutic drugs (e.g., cycloheximide and Topotecan) is able to kill TRAIL receptor expressing cells in a synergistic manner.

Other chemotherapeutics that may be tested in this assay (and used in treatment regimens in conjunction with the antibodies of the present invention) include, for example, camptothecin, irinotecan (camptosar, CPT-11), adraimycin (doxorubicin), methotrexate, paraplatinin, interferon-alpha, paclitaxel, docetaxel, the NF-kappa-B inhibitor SN50, and gemcitabine (Gemzar™). Other cell lines that may be tested in this assay include, for example, the human Burkitt lymphoma line ST486, human breast carcinoma cell line MDA-MB-231, the human uterine carcinoma cell line RL-95, the human lung carcinoma cell line SK-MES-1, human colon cancer cell lines, LS174T, HT29, and HCT116, the su.86.86 and CFPAC pancreatic cancer cell lines, the human ovarian cancer cell line TOV21G, and the human hepatocellular cancer cell line SNU449. Cancers of the tissues corresponding to the tissues from which these cancer cell lines were derived may be treated with the therapeutic compositions in accordance with the invention.

In addition, the assay described in this example may also be used to test the effect of more than one anti-TRAIL receptor antibody on TRAIL Receptor expressing cells. For example, cells may be treated with both an antibody that specifically binds TR4 and an antibody that specifically binds TR7. As above, this experiment may be performed in the presence of absence of one or more chemotherapeutic agents or crosslinking agents. In another variation of the present experiment antibodies of the invention may tested for the apoptosis inducing effect when used in the presence of TRAIL. The amount of apoptosis induced by dual treatment with anti-TR4 and anti-TR7 may be synergistic compared to treatment with either anti-TR4 or anti-TR7 alone. Such an effect may be more pronounced when the experiment is performed in the presence of chemotherapeutic and/or crosslinking agents.

Antibodies from hybridomas produced in Xenomouse Fusion XF-2 were tested in an assay similar to the above described Alamar Blue assay for their ability to induce apoptosis of ST486 cells in the presence or absence of crosslinking. In this assay 5×10⁵ cells were used. When tested in the absence of a crosslinking agent, the antibodies were used at a concentration of 10 micrograms/ml; when tested in the presence of a crosslinking agent, the antibodies were used at a concentration of 1 microgram/ml and the crosslinking anti-human IgG was used at a concentration of 10 micrograms/ml. Table 8 presents EC-50 data for these clones' ability to induce apoptosis of ST486 cells.

TABLE 8
EC-50 Values for Antibody Ability
to Induce Apoptosis of ST486 Cells

| | EC-50 with | EC50 without |
|---|---|---|
| Clone ID | crosslinking (M) | crosslinking (M) |
| XF2-1A12 | 1.72 × 10⁻¹¹ | 2.66 × 10⁻¹² |
| XF2-1G3 | 8.97 × 10⁻¹² | 2.07 × 10⁻¹¹ |
| XF2-1G5 | >1.0 × 10⁻⁸ | >1.0 × 10⁻⁸ |
| XF2-2D2 | >1.0 × 10⁻⁸ | >1.0 × 10⁻⁸ |
| XF2-2H1 | 1.00 × 10⁻¹¹ | 1.50 × 10⁻¹⁰ |
| XF2-4C2 | 1.00 × 10⁻¹¹ | 8.70 × 10⁻¹² |
| XF2-4F7 | 2.78 × 10⁻¹⁰ | 4.82 × 10⁻¹¹ |
| XF2-4G8 | 2.81 × 10⁻¹⁰ | 7.39 × 10⁻¹¹ |
| XF2-4H5 | 7.64 × 10⁻¹² | 1.48 × 10⁻¹¹ |
| XF2-4H11 | 2.59 × 10⁻¹⁰ | 4.94 × 10⁻¹⁰ |
| XF2-18A10 | 1.65 × 10⁻⁹ | >1.0 × 10⁻⁸ |
| XF2-19C10 | 4.05 × 10⁻⁹ | >1.0 × 10⁻⁸ |
| XF2-23H7 | 4.87 × 10⁻¹⁰ | >1.0 × 10⁻⁸ |

### Example 5

Identification and Cloning of VH and VL Domains

One method to identfy and clone VR and VL domains from cell lines expressing a particular antibody is to perform PCR with VH and VL specific primers on cDNA made from the antibody expressing cell lines. Briefly, RNA is isolated from the cell lines and used as a template for RT-PCR designed to amplify the VR and VL domains of the antibodies expressed by the EBV cell lines. Cells may lysed in the TRIzol® reagent (Life Technologies, Rockville. Md.) and extracted with one fifth volume of chloroform. After addition of chloroform, the solution is allowed to incubate at room temperature for 10 minutes, and the centrifuged at 14,000 rpm for 15 minutes at 4° C. in a tabletop centrifuge. The supernatant is collected and RNA is precipitated using an equal volume of isopropanol. Precipitated RNA is pelleted by centrifuging at 14,000 rpm for 15 minutes at 4° C. in a tabletop centrifuge. Following centrifugation, the supernatant is discarded and washed with 75% ethanol. Follwing washing, the RNA is centrifuged again at 800 rpm for 5 minutes at 4° C. The supernatant is discarded and the pellet allowed to air dry. RNA is the dissolved in DEPC water and heated to 60° C. for 10 minutes. Quantities of RNA can determined using optical density measurements.

cDNA may be synthesized, according to methods well-known in the art, from 1.5-2.5 micrograms of RNA using reverse transciptase and random hexamer primers. cDNA is then used as a template for PCR amplification of VH and VL domains. Primers used to amplify VH and VL genes are shown in Table 9. Typically a PCR reaction makes use of a single 5′ primer and a single 3′ primer. Sometimes, when the amount of available RNA template is limiting, or for greater efficiency, groups of 5′ and/or 3′ primers may be used. For example, sometimes all five VH-5′ primers and all JH3′ primers are used in a single PCR reaction. The PCR reaction is carried out in a 50 microliter volume containing 1×PCR buffer, 2 mM of each dNTP, 0.7 units of High Fidelity Taq polymerse, 5′ primer mix, 3′ primer mix and 7.5 microliters of cDNA. The 5′ and 3′ primer mix of both VH and VL can be made by pooling together 22 pmole and 28 pmole, respectively, of each of the individual primers. PCR conditions are: 96° C. for 5 minutes; followed by 25 cycles of 94° C. for 1 minute, 50° C. for 1 minute, and 72° C. for 1 minute; followed by an extension cycle of 72° C. for 10 minutes. After the reaction is completed, sample tubes were stored 4° C.

TABLE 9

Primer Sequences Used to Amplify VH and VL
domains.

| | | |
|---|---|---|
| Primer name | SEQ ID NO | Primer Sequence (5′-3′) |

| | | |
|---|---|---|
| VH Primers | | |
| Hu VH1-5′ | 6 | CAGGTGCAGCTGGTGCAGTCTGG |
| Hu VH2-5′ | 7 | CAGGTCAACTTAAGGGAGTCTGG |
| Hu VH3-5′ | 8 | GAGGTGCAGCTGGTGGAGTCTGG |
| Hu VH4-5′ | 9 | CAGGTGCAGCTGCAGGAGTCGGG |
| Hu VH5′-5′ | 10 | GAGGTGCAGCTGTTGCAGTCTGC |
| Hu VH6-5′ | 11 | CAGGTACAGCTGCAGCAGTCAGG |
| Hu JH1,2-5′ | 12 | TGAGGAGACGGTGACCAGGGTGCC |
| Hu JH3-5′ | 13 | TGAAGAGACGGTGACCATTGTCCC |
| Hu JH4,5-5′ | 14 | TGAGGAGACGGTGACCAGGGTTCC |
| Hu JH6-5′ | 15 | TGAGGAGACGGTGACCGTGGTCCC |
| VL Primers | | |
| Hu Vkappa1-5′ | 16 | GACATCCAGATGACCCAGTCTCC |
| Hu Vkappa2a-5′ | 17 | GATGTTGTGATGACTCAGTCTCC |
| Hu Vkappa2b-5′ | 18 | GATATTGTGATGACTCAGTCTCC |
| Hu Vkappa3-5′ | 19 | GAAATTGTGTTGACGCAGTCTCC |
| Hu Vkappa4-5′ | 20 | GACATCGTGATGACCCAGTCTCC |
| Hu Vkappa5-5′ | 21 | GAAACGACACTCACGCAGTCTCC |
| Hu Vkappa6-5′ | 22 | GAAATTGTGCTGACTCAGTCTCC |
| Hu Vlambda1-5′ | 23 | CAGTCTGTGTTGACGCAGCCGCC |
| Hu Vlambda2-5′ | 24 | CAGTCTGCCCTGACTCAGCCTGC |
| Hu Vlambda3-5′ | 25 | TCCTATGTGCTGACTCAGCCACC |
| Hu Vlambda3b-5′ | 26 | TCTTCTGAGCTGACTCAGGACCC |
| Hu Vlambda4-5′ | 27 | CACGTTATACTGACTCAACCGCC |
| Hu Vlambda5-5′ | 28 | CAGGCTGTGCTCACTCAGCCGTC |
| Hu Vlambda6-5′ | 29 | AATTTTATGCTGACTCAGCCCCA |
| Hu Jkappa1-3′ | 30 | ACGTTTGATTTCCACCTTGGTCCC |
| Hu Jkappa2-3′ | 31 | ACGTTTGATCTCCAGCTTGGTCCC |
| Hu Jkappa3-3′ | 32 | ACGTTTGATATCCACTTTGGTCCC |
| Hu Jkappa4-3′ | 33 | ACGTTTGATCTCCACCTTGGTCCC |
| Hu Jkappa5-3′ | 34 | ACGTTTAATCTCCAGTCGTGTCCC |
| Hu Jlambda1-3′ | 35 | CAGTCTGTGTTGACGCAGCCGCC |
| Hu Jlambda2-3′ | 36 | CAGTCTGCCCTGACTCAGCCTGC |
| Hu Jlambda3-3′ | 37 | TCCTATGTGCTGACTCAGCCACC |
| Hu Jlambda3b-3′ | 38 | TCTTCTGAGCTGACTCAGGACCC |
| Hu Jlambda4-3′ | 39 | CACGTTATACTGACTCAACCGCC |
| Hu Jlambda5-3′ | 40 | CAGGCTGTGCTCACTCAGCCGTC |
| Hu Jlambda6-3′ | 41 | AATTTTATGCTGACTCAGCCCCA |

PCR samples are then electrophoresed on a 1.3% agarose gel. DNA bands of the expected sizes (−506 base pairs for VH domains, and 344 base pairs for VL domains) can be cut out of the gel and purified using methods well known in the art. Purified PCR products can be ligated into a PCR cloning vector (TA vector from Invitrogen Inc., Carlsbad, Calif.). Individual cloned PCR products can be isolated after transfection of E. coli and blue/white color selection. Cloned PCR products may then be sequenced using methods commonly known in the art.

### Example 6

### Anti-TRAIL Receptor Antibodies Retard the Growth of Tumor Cells in Nude Mice

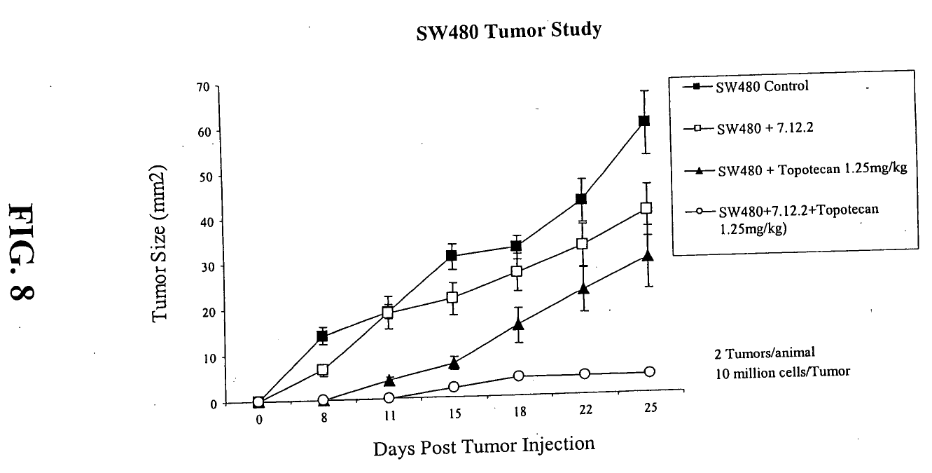

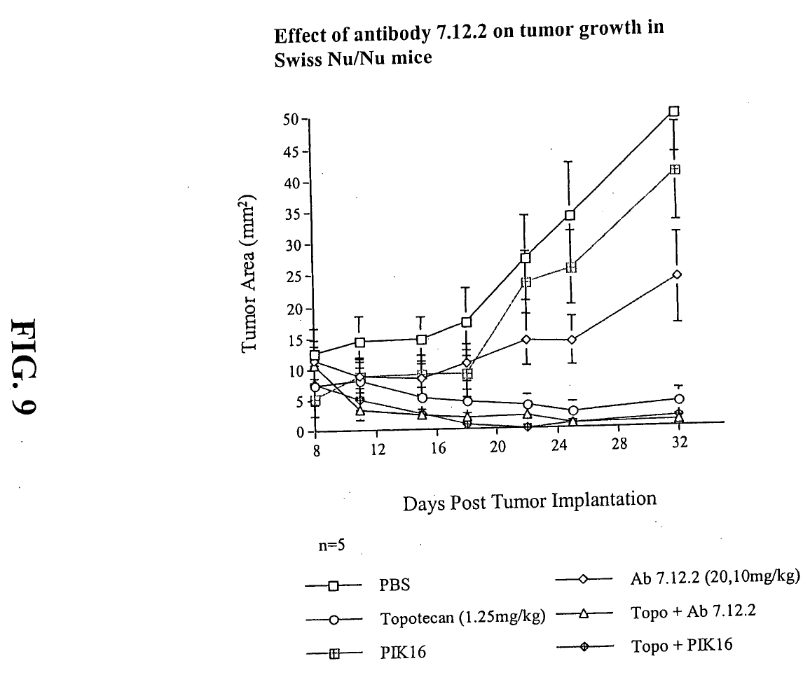

SW480 (colorectal adenocarcinoma) tumor cell line was maintained in vitro in Leibovitz's L-15 medium supplemented with fetal bovine serum, glutamine and antibiotics as per the instructions received from American Type Culture Collection. Cells at passage 3-10 were used for the in vivo studies. The tumor cells were harvested from the T-150 flasks, rinsed with sterile PBS and then resuspended in sterile saline at a density of 5(104) cells/ul. Tumor cells were implanted subcutaneously on the upper back or flanks of Swiss athymic mice at a density of 10⁷ cells per site, 2 sites per animal. In preventive (de novo) tumor models, chemotherapeutic agents and antibody treatments were initiated 24 hr post-tumor cell inoculation. The antibody treatment, 7.12.2, was as follows: loading dose: 20 mg/kg, intavenously 24 hours post injection of tumor cells, maintenance dose: 10 mg/kg, intraperitoneally every third day thereafter for a total of five injections. Topotecan was the chemotherapeutic agent used in this experiment. The dose and dosing frequency are as follows: 1.25 mg/kg, intraperitoneally on the first, second, third, fourth, seventh, fourteenth, and eighteenth days of the experiment. The endpoint for the experiment was tumor size. Tumors were measured on 2 axes with a caliper two times per week. When 7.12.2 was administered with topotecan a significant reduction in tumor size was observed. Treatment with the antibody alone may reduce tumor growth at the later time points. (See FIG. 8). In a repeat of this experiment, in which the same loading doase of antibody was given on day one followed by the same maintenance doses of antibody on days four, seven, eleven, fourteen, eighteen, twenty-one and twent-five. Topotecan treatment (1.25 mg/kg) was given on days one through four and on days, seven, eleven, fourteen, eighteen, twenty-one and twent-five. In this experiment a control antibody PIK16 of irrelevant specifiicty and IgG2 isotype was also included (See FIG. 9).

A similar experiment may be performed to asses the effect of treatment with anti-TRAIL receptor antibodies on the growth of pre-established tumors by injecting the tumors and allowing them to grow to a particular size (e.g. 5×5 mm tumor size).

The above described assay may also be used to test the effect of treatment with more than one anti-TRAIL receptor antibody on the growth of tumor cells in vivo. For example, animals into which tumor cells have been injected may be treated with both an antibody that specifically binds TR4 and an antibody that specifically binds TR7. As above, this experiment may be performed in the presence of absence of one or more chemotherapeutic agents. In another variation of the present experiment antibodies of the invention may administered in combination with TRAIL. The ability of such combination therapy to inhibit the growth of tumor cells as compared to treatment with either antibody alone can be assayed using the methods detailed above, and comparing the results obtained between the combination therapy with the results obtained from treatment with either an anti-TR4 or an anti-TR7 antibody alone.

### Example 7

### Immunohistochemistry of Primary Tumor Tissue for Expression of TRAIL R1 (TR4) Expression

Primary human tumor tissues of the bladder, breast, colon, liver lung ovary and pancreas were stained with a goat anti-human TRAIL-R1 polyclonal antibody (R&D Systems). This antibody stains cells transfected with TRAIL-R1 expression constructs, but not vector control transfected cells. Staining data are presented below in Table 10 below. Positive staining was observed in certain breast, colon, lung, and stomach carcinoma tissues. In contrast, normal human tissue samples from the same organs, had no specific staining. In addition, no specific staining was observed in normal human and monkey liver and spleen samples.

TABLE 10
Immunohistochemical staining of Human tumor and Normal Tissues

| | # Evaluated | Positive | +/− | Negative |
|---|---|---|---|---|
| Tumor Tissue | | | | |
| Bladder | 2 | 0 | 1 | 1 |
| Breast | 2 | 1 | 0 | 1 |
| Colon | 2 | 1 | 1 | 0 |
| Liver | 2 | 0 | 1 | 1 |
| Lung | 2 | 2 | 0 | 1 |
| Ovary | 1 | 0 | 0 | 1 |
| Pancreas | 2 | 0 | 0 | 2 |
| Stomach | 1 | 1 | 0 | 0 |
| Totals | 14 | 5 | 3 | 6 |
| Normal Tissue | | | | |
| Bladder | 1 | 0 | 0 | 1 |
| Breast | 0 | 0 | 0 | 0 |
| Colon | 1 | 0 | 0 | 1 |
| Liver | 1 | 0 | 1 | 0 |
| Lung | 1 | 0 | 0 | 1 |
| Ovary | 1 | 0 | 0 | 1 |
| Pancreas | 1 | 0 | 0 | 1 |
| Stomach | 0 | 0 | 0 | 0 |
| Totals | 6 | 0 | 1 | 5 |

### Example 8

### Antibody Production and Purification

The following example describes a large scale antibody production and purification methods that may be used to make antibodies of the present invention. One of skill in the art will be aware of routine modifications to the protocol described below, for example, as regards column choice, column, loading, wash, and elution buffers, and pH.

Cell Culture Scale-Up and Antibody Production

A serum-free and animal source-free growth medium (HGS-NS0SF) is used from thawing cells through scale-up to the production bioreactor. The HGS-NS0SF growth medium is prepared by adding 20 mL/L GS supplement and 1 mL/L cholesterol (synthetic) lipid concentrate into 1 L CD hybridoma media without 1-glutamine (Invitrogen/Life technologies). The media are stored at 2-8° C. until use.

Thawing Cells from MCB Vial(s)

Approximately 16×10⁶ cells are thawed at 37° C. in a water bath. The cells are transferred into T-225 culture flask(s) to yield approximately 50 mL working volume with an inoculation density of approximately 3.0×10⁵ cells/mL. The culture flask(s) is then placed in a humidified CO₂ incubator at 37° C. with 5% CO₂ for 4 days.

First Expansion(s) of Culture in Spinner Flask

The culture is aseptically expanded into a 500 mL spinner flask to give approximately 300 mL working volume, at an inoculation cell density of approximately 2.2×10⁵ cells/mL. The spinner flask is then placed on magnetic stirrers in a humidified CO₂ incubator at 37° C. with 5% CO₂ for 4 days. The agitation rate for the spinner flask is 80 rpm.

The culture is again expanded aseptically into one 3000 mL spinner flask to give approximately 1500 mL working volume, at an inoculation cell density of approximately 2.2×10⁵ cells/mL. The spinner flask is then placed on magnetic stirrers in a humidified CO₂ incubator at 37° C. with 5% CO₂ for 4 days. The agitation rate for the spinner flasks is 80 rpm. If a sufficient amount of cell culture is accumulated to inoculate the seed bioreactor, proceed to Step 4. If not, the culture is expanded aseptically into multiple 3000 mL spinner flasks for a total of 3 to 4 expansions, until a sufficient amount of cell culture is accumulated to inoculate the seed bioreactor.

Seed Culture

The seed bioreactor is equipped with 2 impellers for mixing, a dissolved oxygen probe, a temperature probe, a pH probe, aseptic sampling and additional systems. The first step of the cell cultivation process is the addition of HGS-NS0SF media into the bioreactor. After the HGS-NS0SF media temperature reaches 37±0.5° C., the dissolved oxygen (DO) and pH levels are stabilized by addition of N₂ and CO₂ to decrease dissolved oxygen concentration to 30±5% air saturation, and obtain a pH of 7.20±0.10. The agitation rate is 80 rpm. The pooled cell culture is transferred aseptically to a 15 L seed bioreactor containing sterile HGS-NS0SF growth media to yield a culture with an inoculation cell density of approximately 2.2×10⁵ cells/mL. During the cultivation process the temperature is maintained via a heat blanket and a cooling finger, the oxygen concentration is maintained via sparger and surface aeration, and pH is controlled by addition of CO₂ gas to lower the pH. The cultivation period is 5-6 days. The bioreactor air vents are protected by hydrophobic 0.2 μm vent filters.

Production Culture

The production bioreactor is equipped with 2 impellers for mixing, 2 dissolved oxygen probes, a temperature probe, 2 pH probes, aseptic sampling and additional systems. 80 L of HGS-NS0SF growth media is aseptically transferred into the 100 L production bioreactor. After the HGS-NS0SF growth media temperature reaches 37±0.5° C., the DO and pH levels are stabilized by addition of N₂ and CO₂ to decrease dissolved oxygen concentration to 30±5% air saturation, and obtain a pH of 7.20±0.10. The agitation rate is 45 rpm. The 15 L seed culture is aseptically transferred into the production bioreactor to yield a culture with an inoculation cell density of approximately 2.2×10⁵ cells/mL. During the cultivation process the temperature is maintained via a heat exchanger, the oxygen concentration is maintained via sparger and surface aeration, and pH is controlled by addition of CO₂ gas to lower the pH. On day 3 after inoculation when cell density reaches approximately 1.0×10⁶ cells/mL, approximately 6 L of HGS-NS0SF fed-batch media was fed into the production bioreactor. The production culture containing the antibody was harvested on Day 5 after feeding.

Recovery and Purification

Harvest of Cell Supernatant

Cell supernatant, (e.g., culture supernatant from NSO cells expressing antibodies of the invention) is harvested on day 5 or 6 post final feeding in the final production bioreactor using a fed-batch cell culture process. The harvest process is started when the antibody concentration of at least 400 mg/L is attained. Cell culture temperature in the production bioreactor is cooled down to 15° C. at the time of harvest and maintained at that temperature during the recovery. A depth filtration process is used for cell removal and antibody recovery. The filtration process train consists of 4.5 μm, 0.45 μm and 0.2 μm pore size filters connected in series. A constant flow rate of 1.00 L/min is maintained during the operation with a cross-filter-pressure control of up to 15 psi. The 0.2 μm filtered culture supernatant is collected in a process bag and transferred for purification.

The purification process is conducted at 22 to 26° C.

Chromatography on MEP HyperCEL HCIC Column

The culture supernatant is loaded onto a MEP HyperCEL™ column, a Hydrophobic charge interaction chromatography, HCIC, available from Ciphergen Biosystems, or equivalent column that is equilibrated in 50 mM Tris, 0.5 M sodium chloride, pH 7.5. The MEP column is washed with 25 mM sodium citrate, 0.15 M sodium chloride, pH 6.4 and eluted with 25 mM sodium citrate, 0.15 M sodium chloride, pH 4.4. The elution is monitored by ultraviolet (UV) absorbance at 280 nm. The peak fractions are collected, analyzed by A₂₈₀ and SDS-PAGE. Appropriate fractions are pooled. Alternatively, the chromatography on the a MEP HyperCEL™ may be substituted with affinity chromatography over a recombinant protein A column.

Virus Inactivation

The eluate from the MEP column is adjusted with 1 M citric acid to pH 3.4±0.2 and allowed to stand for 45-60 minutes for viral inactivation. The solution is then re-adjusted to pH 5.0 with 1 M Tris base.

Chromatography on SP Sepharose FF Column

The inactivated eluate from the MEP column is diluted with water for injection (WFI) to a conductivity of 5 mS/cm, and loaded onto a SP Sepharose FF (cation exchange chromatography, Amersham-Pharmacia) column, or equivalent column equilibrated with 65 mM sodium acetate, pH 5.0. The antibody is eluted from the SP column with 20 mM sodium citrate, 0.15 M sodium chloride, 1.9% glycine, pH 7.1. The elution is monitored by ultraviolet (UV) absorbance at 280 nm. Peak fractions are collected and analyzed by A₂&O and SDS-PAGE. Appropriate fractions are pooled.

Virus Removal Filtration, Diafiltration and Concentration

The eluate from the SP Sepharose FF column is filtered through a sequentially connected 0.2 μm filter and a Pall DV50 viral removal filter. The DV50 filtrate is placed into a 30 kD MW cut-off membrane device (Millipore Pellicon) to concentrate to a target concentration of 35-40 mg/mL, and diafiltered against 10 mM sodium citrate, 1.9% glycine, 0.5% sucrose, pH 6.5. The diafiltered material is monitored by A₂₈₀. The diafiltered bulk is 0.2 μm filtered and stored at 2-8° C. up to 24 hours.

Chromatography on Q Sepharose FF Column

The diafiltered TRM-1 solution is passed over a Q Sepharose FF column (anion exchange chromatography, Amersham-Pharmacia) or equivalent column equilibrated with 10 mM sodium citrate, 1.9% glycine, 0.5% sucrose, pH 6.5. The antibody is collected in the flow-through and monitored by A₂₈₀. Appropriate fractions are pooled and the final target concentration is 25 mg/mL.

Bulk Formulation, Filtration and Bulk Drug Substance Fill

Polysorbate 80 (2% stock solution) is pre-filtered through a 0.2 μm filter and added to the antibody solution from step 7 to a final concentration of 0.02%. The purified antibody is aseptically filtered under a laminar flow hood through a 0.2 μm filter and filled into polypropylene containers.

Storage of Bulk Drug Substance

The bulk drug substance is stored at 2-8° C. (short-term storage) or at or below −65° C. (long-term storage) prior to the release of the product. In-process testing of the unprocessed production bioreactor culture at harvest for each batch and in-process testing during the purification process are performed. The bioreactor is sampled aseptically and the culture is tested at various times throughout cultivation for cell density, viability and nutrient determination to ensure consistency of material being supplied for purification. The purification process is monitored at each step. Appearance is checked by visual inspection. The protein concentration is determined by Absorbance at 280 nm. The pH of the material is checked. Purity is checked, for example, by SDS-PAGE and size exclusion chromatography. An ELISA may be performed to check the ability of the antibody to bind its antigen. The biological activity of the antibody is also monitored. Residual DNA content, Endotoxin levels, and the bioburden (the number of viable organisms present in the antibody preparation) are all monitored and kept at or below standard acceptable levels. Additionally the oligosaccharide content may be analyzed; the peptide sequence of the antibody chains may also be analyzed using N-terminal sequencing and peptide mapping. Short and long-term studies of antibody stability may also be performed.

### Example 9

### Effect of Anti-TR7 Antibodies on Human Hepatocytes

The effect of anti-TR7 antibodies on human primary hepatocytes can be determined by measuring either caspase activation or cell viability. The following is an exemplary assay to determine the efeect of anti-TR7 antibodies in caspase activation in human hepatocyte and heaptocyte viability. Human hepatocytes are treated with 15.6, 62.5, 250 or 1000 ng/mL of TRAIL (amino acid residues 114-281, Biomol Research Laboratories Inc, Plymouth Meeting, Pa.), 62.5, 125, 250, or 1000 ng/ml of isotype control mAb (hIgG₁, CAT002) or 62.5, 125, 250, or 1000 ng/ml anti-TR7 antibody. Caspase activation is determined at 6 hrs following treatment, while viability is determined at 24 hrs following treatment.

Caspase activity can be measured using a fluorimetric assay utilizing the caspase substrate Rhodamine conjugated DEVD, (e.g., Homogeneous Fluorimetric Caspases Assay available from Roche Molecular Biochemicals (Indianapolis, Ind.)). Cell viability was determined using an ALAMAR Blue™ (Biosource International, Camarillo, Calif.) assay. TRAIL reduced cell viability at all concentrations tested, and induced caspase activity at the highest concentration tested.

Apoptotic signaling may be measured by any assay described herein or otherwise known in the art. For example, apoptosis or apoptotic signaling, such as that induced by agonistic anti-TR4 antibodies, may also be measured or monitored using terminal dUTP nick end labeling (TUNEL) assay, immunohistochemistry, Western blot analysis, real-time RT-PCR, and enzyme activity assay. One such Western Blot analysis that could be performed is described below. Approximately 2×10⁶ cells are plated in 150-mm cell culture plates and cultured overnight. Cells are then treated with various concentrations of chemotherapeutic drugs, antibody crosslinking agent (such as a goat anti-human IgG antibody) and or agonistic antibody for a given length of time (e.g., minutes, hours or days). After stimulation, the cells are scraped from the plate in ice-cold PBS and lysed with 1% NP40 lysis buffer (10 mM HEPES) pH7.5, 0.15 mM NaCl, 10% glycerol, protease inhibityor cocktail and 1 mM EDTA). The protein concentration of the lysates is determined, for example, by the CBA method (Pierce) and equalized with lysis buffer. The proteins are separated using a 10% or a 4-20% gradient polyacrylamide SDS gel electrophoresis and transferred to nitrocellulose memebrane. The membranes are immunoblotted with antibodies to different protins according to standard western blotting protocols. Proteins involved associated with cell survival and/or apoptosis that may be assessed include, but are not limited to Poly(ADP-ribose) polymerase (PARP), caspase-8, caspase-3, caspase 9, and BID. Upon induction of an apoptosis signaling cascade, PARP is cleaved to a lower molecular weight form as are caspases-3, -8 and 9. Upon induction of an apoptosis signaling cascade caspase 8 cleaves BID and the COOH-terminal part of BID translocates to mitochondria where it triggers cytochrome c release. Each of the cleavage events may be monitored by western blotting.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference.

Further, the Sequence Listing submitted herewith, in both computer and paper forms, is hereby incorporated by reference in its entirety.

The entire disclosure (including the specification, sequence listing, and drawings) of each of the following U.S. applications are herein incorporated by reference in their entirety: U.S. Provisional Application Ser. Nos. 60/608,449, filed Sep. 10, 2004; 60/495,139, filed Aug. 15, 2003; 60/486,105 filed May 6, 2003; 60/403,376 filed Aug. 15, 2002; 60/377,973 filed May 7, 2002; 60/331,309 filed Nov. 14, 2001; 60/327,359 filed Oct. 9, 2001; 60/295,018 filed Jun. 4, 2001; 60/252,904 filed Nov. 27, 2000; 60/248,847 filed Nov. 16, 2000, 60/246,612 filed Nov. 8, 2000 and U.S. application Ser. No. 09/986,149 filed Nov. 7, 2001; Ser. No. 10/292,486 filed Nov. 13, 2002; and Ser. No. 10/838,977 filed May 5, 2004.

What is claimed is:

1. An isolated antibody comprising an amino acid sequence selected from the group consisting of:
(a) an amino acid sequence that is at least 90% identical to a VH domain of any one of SEQ ID NOS:42, 44, or 46;
(b) an amino acid sequence that is at least 90% identical to a VL domain of any one of SEQ ID NOS:43, 45, or 47; and
(c) both (a) and (b);
wherein said antibody immunospecifically binds to TR4.

2. The antibody of claim 1 wherein the amino acid sequence is selected from the group consisting of:
(a) the amino acid sequence of a VH domain of any one of SEQ ID NOS:42, 44, or 46;
(b) the amino acid sequence of a VL domain of any one of SEQ ID NOS:43, 45, or 47; and
(c) both (a) and (b).

3. The antibody of claim 1 wherein the antibody is selected from the group consisting of:
(a) a whole immunoglobulin molecule;
(b) an scFv;
(c) a monoclonal antibody;
(d) a human antibody;
(e) a chimeric antibody;
(f) a humanized antibody;
(g) a Fab fragment;
(h) an Fab′ fragment;
(i) an F(ab′)2;
(j) an Fv; and
(k) a disulfide linked Fv.

4. The antibody of claim 1 wherein the antibody is multimeric.

5. The antibody of claim 1 wherein the antibody is crosslinked.

6. The antibody of claim 1 wherein the antibody has a dissociation constant (K_{D}) selected from the group consisting of:
(a) a dissociation constant (K_{D}) between 10⁻⁷ M and 10⁻⁸ M;
(b) a dissociation constant (K_{D}) between 10⁻⁸ M and 10⁻⁹ M;
(c) a dissociation constant (K_{D}) between 10⁻⁹ M and 10⁻¹⁰ M;
(d) a dissociation constant (K_{D}) between 10⁻¹⁰ M and 10⁻¹¹ M;
(e) a dissociation constant (K_{D}) between 10⁻¹¹ M and 10⁻¹² M; and
(f) a dissociation constant (K_{D}) between 10⁻¹² M and 10⁻¹³ M.

7. The antibody of claim 1 wherein the antibody is associated with a detectable label.

8. The antibody of claim 1 wherein the antibody is conjugated to a therapeutic or cytotoxic agent.

9. The antibody of any one of claim 1 which is attached to a solid support.

10. An isolated cell that produces the antibody of claim 1.

11. The antibody of claim 1 wherein the antibody is an agonist of a TR4 receptor.

12. The antibody of claim 1 wherein the antibody is an antagonist of TR4.

13. A method of treating, preventing or ameliorating a cancer comprising administering the antibody of claim 1 or a composition containing the antibody of claim 1 to an animal.

14. The method of claim 13, wherein the antibody is administered in combination with a chemotherapeutic agent.

15. A method of inhibiting the growth of or killing TR4 expressing cells, comprising administering to an animal in which such inhibition of growth or killing of TR4 receptor expressing cells is desired, the antibody of claim 1 or a composition containing the antibody of claim 1 in an amount effective to inhibit the growth of or kill TR4 expressing cells.

16. A method of detecting expression of a TR4 polypeptide comprising:
(a) assaying the expression of a TR4 polypeptide in a biological sample from an individual using the antibody of claim 1; and
(b) comparing the level of a TR4 polypeptide with a standard level of a TRAIL receptor polypeptide.

17. A method of detecting, diagnosing, prognosing, or monitoring cancers and other hyperproliferative disorders comprising:
(a) assaying the expression of a TR4 polypeptide in a biological sample from an individual using the antibody of claim 1; and
(b) comparing the level of a TR4 polypeptide with a standard level of TR4 polypeptide.

18. A kit comprising the antibody of claim 1.

19. A hybridoma cell line selected from XF2-1A12, XF2-1G3, XF2-1G5, XF2-2D2, XF2-2H1, XF24C2, XF2-4F7, XF24G8, XF24H5, XF2-4H11, XF2-18A10, XF2-19C10, or XF2-23H7, or the hybridoma cell lines contained in ATCC Deposit Nos. PTA-3149, PTA-2687, PTA-3369, PTA-2730, PTA-2729, PTA-2728, PTA-3368, and PTA-2731.

20. An antibody that binds TR4 selected from the group consisting of:
(a) the antibody expressed by the 7.1.3 cell line contained in ATCC Deposit No. PTA-3149;
(b) the antibody expressed by the 7.3.1 cell line contained in ATCC Deposit No. PTA-2687;
(c) the antibody expressed by the 7.3.2 cell line contained in ATCC Deposit No. PTA-2687;
(d) the antibody expressed by the 7.3.3 cell line contained in ATCC Deposit No. PTA-3369;
(e) the antibody expressed by the 7.8.1 cell line contained in ATCC Deposit No. PTA-2730;
(f) the antibody expressed by the 7.8.2 cell line contained in ATCC Deposit No. PTA-2730;
(g) the antibody expressed by the 7.8.3 cell line contained in ATCC Deposit No. PTA-2730;
(h) the antibody expressed by the 7.10.1 cell line contained in ATCC Deposit No. PTA-2729;
(i) the antibody expressed by the 7.10.2 cell line contained in ATCC Deposit No. PTA-2729;
(j) the antibody expressed by the 7.10.3 cell line contained in ATCC Deposit No. PTA-2729;
(k) the antibody expressed by the 7.12.1 cell line contained in ATCC Deposit No. PTA-2728;
(l) the antibody expressed by the 7.12.3 cell line contained in ATCC Deposit No. PTA-2728;
(m) the antibody expressed by the 8.3.1 cell line contained in ATCC Deposit No. PTA-2731;
(n) the antibody expressed by the 8.3.2 cell line contained in ATCC Deposit No. PTA-2731;
(o) the antibody expressed by primary hybridoma isolate XF2-1A12;
(p) the antibody expressed by primary hybridoma isolate XF2-1G3;
(q) the antibody expressed by primary hybridoma isolate XF2-1G5;
(r) the antibody expressed by primary hybridoma isolate XF2-2D2;
(s) the antibody expressed by primary hybridoma isolate XF2-2H1;
(t) the antibody expressed by primary hybridoma isolate XF2-4C2;
(u) the antibody expressed by primary hybridoma isolate XF2-4F7;
(v) the antibody expressed by primary hybridoma isolate XF24G8;
(w) the antibody expressed by primary hybridoma isolate XF24H5;
(x) the antibody expressed by primary hybridoma isolate XF24H11;
(y) the antibody expressed by primary hybridoma isolate XF2-18A10;
(z) the antibody expressed by primary hybridoma isolate XF2-19C10; and
(aa) the antibody expressed by primary hybridoma isolate XF2-23H7.
